(19) United States
(12) Patent Application Publication
(10) Pub. No.: US 2010/0305093 A1
(43) Pub. Date: Dec. 2, 2010

(54) Inhibitors of mTOR and Methods of Making and Using

(75) Inventors: Neel Kumar Anand, Burlingame, CA (US); Arlyn Arcalas, South San Francisco, CA (US); Charles M. Blazey, San Francisco, CA (US); Chris A. Buhr, Redwood City, CA (US); Jonah Cannoy, Fremont, CA (US); Sergey Ephsteyn, Fremont, CA (US); Henry William Beecroft Johnson, San Bruno, CA (US); Anagha Joshi, Fremont, CA (US); Byung Gyu Kim, Foster City, CA (US); James W. Leahy, San Leandro, CA (US); Matthew Sangyup Lee, Millbrae, CA (US); Sunghoon Ma, Foster City, CA (US); Morrison B. Mac, San Francisco, CA (US); John M. Nuss, Danville, CA (US); Craig Stacy Takeuchi, Burlingame, CA (US); Longcheng Wang, Palo Alto, CA (US); Yong Wang, Foster City, CA (US)

Correspondence Address: MCDONNELL BOEHNEN HULBERT @ BERGHOFF LLP, 300 SOUTH WACKER DRIVE, SUITE 3100, CHICAGO, IL, 60606, US

(73) Assignee: EXELIXIS, INC., South San Francisco, CA (US)

(21) Appl. No.: 12/756,374

(22) Filed: Apr. 8, 2010

Related U.S. Application Data
(60) Provisional application No. 61/212,336, filed on Apr. 9, 2009.

(51) Int. Cl. A61K 31/553; C07D 405/10; A61P 35/00; A61P 37/00; A61P 35/02; A61P 27/00

(52) U.S. Cl. 514/210.21; 540/552; 514/211.09

(57) ABSTRACT

The invention is directed to Compounds of Formula I:

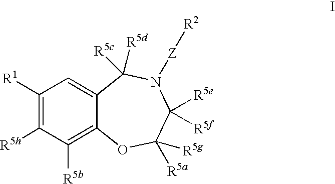

and pharmaceutically acceptable salts or solvates thereof, as well as methods of making and using the compounds.

### BACKGROUND OF THE INVENTION

1. Field of the Invention

This invention relates to the field of protein kinases and inhibitors thereof. In particular, the invention relates to inhibitors of mammalian target of rapamycin (mTOR) signaling pathways, and methods of their use.

2. Background of the Invention

The mammalian target of rapamycin, mTOR, is a protein kinase that integrates both extracellular and intracellular signals of cellular growth, proliferation, and survival. Extracellular mitogenic growth factor signaling from cell surface receptors and intracellular pathways that convey hypoxic stress, energy and nutrient status all converge at mTOR. mTOR exists in two distinct complexes: mTOR complex 1 (mTORC1) and mTOR complex 2 (mTORC2). mTORC1 is a key mediator of transcription and cell growth (via its substrates p70S6 kinase and 4E-BP1) and promotes cell survival via the serum and glucocorticoid-activated kinase SGK, whereas mTORC2 promotes activation of the pro-survival kinase AKT. Given its central role in cellular growth, proliferation and survival, it is perhaps not surprising that mTOR signaling is frequently dysregulated in cancer and other diseases (Bjornsti and Houghton Rev Cancer 2004, 4(5), 335-48; Houghton and Huang Microbiol Immunol 2004, 279, 339-59; Inoki, Corradetti et al. Nat Genet. 2005, 37(1), 19-24).

mTOR is a member of the PIKK (PI3K-related Kinase) family of atypical kinases which includes ATM, ATR, and DNAPK, and its catalytic domain is homologous to that of PI3K. Dyregulation of PI3K signaling is a common function of tumor cells. In general, mTOR inhibition may be considered as a strategy in many of the tumor types in which PI3K signaling is implicated such as those discussed below.

Inhibitors of mTOR may be useful in treating a number of cancers, including the following: breast cancer (Nagata, Lan et al., Cancer Cell 2004, 6(2), 117-27; Pandolfi N Engl J Med 2004, 351(22), 2337-8; Nahta, Yu et al. Nat Clin Pract Oncol 2006, 3(5), 269-280); antle cell lymphoma (MCL) (Dal Col, Zancai et al. Blood 2008, 111(10), 5142-51); renal cell carcinoma (Thomas, Tran et al. Nat Med 2006, 12(1), 122-7; Atkins, Hidalgo et al. J Clin Oncol 2004, 22(5), 909-18; Motzer, Hudes et al. J Clin Oncol 2007, 25(25), 3958-64); acute myelogenous leukemia (AML) (Sujobert, Bardet et al. Blood 2005, 106(3), 1063-6; Billottet, Grandage et al. Oncogene 2006, 25(50), 6648-6659; Tamburini, Elie et al. Blood 2007, 110(3), 1025-8); chronic myelogenous leukemia (CML) (Skorski, Bellacosa et al. Embo J 1997, 16(20), 6151-61; Bai, Ouyang et al. Blood 2000, 96(13), 4319-27; Hickey and Cotter Biol Chem 2006, 281(5), 2441-50); diffuse large B cell lymphoma (DLBCL) (Uddin, Hussain et al. Blood 2006, 108(13), 4178-86); several subtypes of sarcoma (Hernando, Charytonowicz et al. Nat Med 2007, 13(6), 748-53; Wan and Helman Oncologist 2007, 12(8), 1007-18); rhabdomyosarcoma (Cao, Yu et al. Cancer Res 2008, 68(19), 8039-8048; Wan, Shen et al. Neoplasia 2006, 8(5), 394-401); ovarian cancer (Shayesteh, Lu et al. Nat Genet, 1999, 21(1), 99-102; (Lee, Choi et al. Gynecol Oncol 2005, 97(1) 26-34); endometrial tumors (Obata, Morland et al. Cancer Res 1998, 58(10), 2095-7; Lu, Wu et al. Clin Cancer Res 2008, 14(9), 2543-50); non small cell lung carcinoma (NSCLC) (Tang, He et al. Lung Cancer 2006, 51(2), 181-91; Marsit, Zheng et al. Hum Pathol 2005, 36(7), 768-76); small cell, squamous, large cell and adenocarcinoma (Massion, Taflan et al. Am J Respir Crit Care Med 2004, 170(10), 1088-94); lung tumors in general (Kokubo, Gemma et al. Br J Cancer 2005, 92(9), 1711-9; Pao, Wang et al. Pub Library of Science Med 2005, 2(1), e17); colorectal tumors (Velho, Oliveira et al. Eur J Cancer 2005, 41(11), 1649-54; Foukas, Claret et al. Nature, 2006, 441(7091), 366-370), particularly those that display microsatellite instability (Goel, Arnold et al. Cancer Res 2004, 64(9), 3014-21; Nassif, Lobo et al. Oncogene 2004, 23(2), 617-28), KRAS-mutated colorectal tumors (Bos Cancer Res 1989. 49(17), 4682-9; Fearon Ann N Y Acad Sci 1995, 768, 101-10); gastric carcinomas (Byun, Cho et al. Int J Cancer 2003, 104(3), 318-27); hepatocellular tumors (Lee, Soung et al. Oncogene 2005, 24(8), 1477-80); liver tumors (Hu, Huang et al. Cancer 2003, 97(8), 1929-40; Wan, Jiang et al. Cancer Res Clin Oncol 2003, 129(2), 100-6); primary melanomas and associated increased tumor thickness (Guldberg, thor Straten et al. Cancer Res 1997, 57(17), 3660-3; Tsao, Zhang et al. Cancer Res 2000, 60(7), 1800-4; Whiteman, Zhou et al. Int J Cancer 2002, 99(1), 63-7; Goel, Lazar et al. J Invest Dermatol 126(1), 2006, 154-60); pancreatic tumors (Asano, Yao et al. Oncogene 2004, 23(53), 8571-80); prostate carcinoma (Cairns, Okami et al. Cancer Res 1997, 57(22), 4997-5000; Gray, Stewart et al. Br J Cancer 1998, 78(10), 1296-300; Wang, Parsons et al. Clin Cancer Res 1998, 4(3), 811-5; Whang, Wu et al. Proc Natl Acad Sci USA 1998, 95(9), 5246-50; Majumder and Sellers Oncogene 2005, 24(50) 7465-74; Wang, Garcia et al. Proc Natl Acad Sci USA 2006, 103(5), 1480-5; (Lu, Ren et al. Int J Oncol 2006, 28(1), 245-51; Mulholland, Dedhar et al. Oncogene 25(3), 2006, 329-37; Xin, Teitell et al. Proc Natl Acad Sci USA 12006, 03(20), 7789-94; Mikhailova, Wang et al. Adv Exp Med Biol 2008, 617, 397-405; Wang, Mikhailova et al. Oncogene 2008, 27(56), 7106-7117); thyroid carcinoma, particularly in the anaplastic subtype (Garcia-Rostan, Costa et al. Cancer Res 2005, 65(22), 10199-207); follicular thyroid carcinoma (Wu, Mambo et al. J Clin Endocrinol Metab 2005, 90(8), 4688-93); anaplastic large cell lymphoma (ALCL); hamaratomas, angiomyelolipomas, TSC-associated and sporadic lymphangioleiomyomatosis: Cowden's disease (multiple hamaratoma syndrome) (Bissler, McCormack et al. N Engl J Med 2008, 358(2), 140-151); sclerosing hemangioma (Randa M. S. Amin Pathology International 2008, 58(1), 38-44); Peutz-Jeghers syndrome (PJS); head and neck cancer (Gupta, McKenna et al. Clin Cancer Res 2002, 8(3), 885-892); neurofibromatosis (Ferner Eur J Hum Genet. 2006, 15(2), 131-138; Sabatini Nat Rev Cancer 2006, 6(9), 729-734; Johannessen, Johnson et al. Current Biology 2008, 18(1), 56-62); macular degeneration; macular edema; myeloid leukemia; systemic lupus; and autoimmune lymphoproliferative syndrome (ALPS).

Selective inhibition of mTORC1 by rapamycin yields a cytostatic phenotype, arresting cell growth. In contrast, ATP-competitive inhibitors of mTOR are predicted to effectively inhibit not only mTORC1 but also mTORC2, thereby more completely disrupting mitogen, nutrient and stress-mediated, and survival-mediated signaling.

### SUMMARY OF THE INVENTION

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

In view of the important role of mTOR in biological processes and disease states, the inventors realized that inhibitors of this protein kinase, including dual inhibitors mTORC1 and mTORC2 are desirable. Compounds of the Invention are potent and specific inhibitors of mTORC1 and/or mTORC2.

A first aspect of the invention comprises a compound of Formula I:

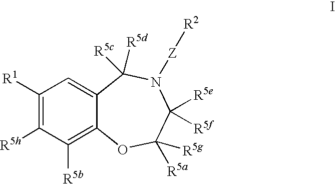

or a single stereoisomer or mixture of isomers thereof and additionally optionally as a pharmaceutically acceptable salt thereof, where
Z is —C(O)—;
R¹ is phenyl optionally substituted with one, two, or three R²⁰ where each R²⁰ is independently nitro; cyano; halo; alkyl; alkenyl; alkynyl; haloalkyl; —NR¹⁵R^{15a}; —NR¹⁵C(O)R¹⁸; —NR¹⁵S(O)₂R¹⁸; —NR¹⁵C(O)NR^{15a}R^{15b}; —OR⁹; —C(O)OR⁹; —C(O)R²⁶; —C(O)NR¹⁶R^{16a}; alkyl substituted with one or two —C(O)NR¹⁶R^{16a}; S(O)₂R¹⁷; heteroaryl optionally substituted with 1, 2, or 3 R²⁷; or optionally substituted heterocycloalkyl; or
R¹ is heteroaryl or an N-oxide thereof, optionally substituted with one, two, or three R²¹, wherein each R²¹ is independently oxo; cyano; alkyl; alkenyl; alkynyl; halo; haloalkyl; hydroxyalkyl; alkoxy; alkoxyalkyl; optionally substituted cycloalkyl; optionally substituted cycloalkylalkyl; optionally substituted heterocycloalkyl; optionally substituted heterocycloalkylalkyl; optionally substituted heteroaryl; optionally substituted heteroarylalkyl; alkyl substituted with phenylalkyloxy; —OR²⁴; —SR²⁵; —S(O)R²⁵; —S(O)₂R²⁵; —S(O)₂NR¹⁵R^{15b}; —C(O)OR²²; —C(O)NR²³R^{23a}; —C(O)R^{24a}; —NR²³R^{23a}; alkyl substituted with one or two —NR²³R^{23a}; —NR²³C(O)OR^{24b}; —NR²³C(O)R^{23a}; alkyl substituted with one or two —NR²³C(O)R^{24a}; —NR²³C(O)NR^{23a}R²⁴; —NR²³C(═NH)NR^{23a}R²⁴; or —NR²³S(O)₂R^{23a};
R² is phenyl or naphthyl, each of which is substituted with R^{3a}, R^{3b}, R^{3c}, and R^{3d}; R² is HET¹ optionally substituted with R^{4a}, R^{4b}, and R^{4c}; or R² is HET² optionally substituted with R^{4a}, R^{4b}, R^{4c}, and R^{4d};
HET¹ is a 5- or 6-membered heteroaryl where the ring atom to which Z is attached is a carbon atom;
HET² is an 8- to 14-membered fused bicyclic ring containing one, two, three, or four ring heteroatoms which are independently O, S, S(O), S(O)₂, or N, with the remaining ring atoms being carbon, where the ring atom attached to Z is carbon and where the ring attached to Z is aromatic and the other ring of HET² is partially or fully unsaturated;
R^{3a}, R^{3b}, R^{3c}, and R^{3d} are independently hydrogen; nitro; cyano; halo; alkyl; alkenyl; alkynyl; cyanoalkyl; haloalkyl; hydroxyalkyl; alkoxyalkyl; haloalkyl substituted with 1, 2, or 3 hydroxy; alkylsulfonylalkyl; —C(O)R²⁸; —C(O)NR¹³R^{13a}; —C(O)C(O)NR²⁹R^{29a}; —SR¹⁴; —S(O)R¹⁹; —S(O)₂R⁶; —S(O)₂NR⁷R^{7a}; —OR⁹; —NR¹¹R^{11a}; alkyl substituted with one or two —NR⁸R^{8a}; optionally substituted phenyl; optionally substituted phenylalkyl; optionally substituted heteroaryl; optionally substituted heteroarylalkyl; optionally substituted heterocycloalkyl; optionally substituted cycloalkyl; or optionally substituted cycloalkylalkyl;
R^{4a}, R^{4b}, R^{4c}, and R^{4d} are independently nitro; cyano; halo; oxo, alkyl; alkenyl; alkynyl; cyanoalkyl; haloalkyl; hydroxyalkyl; alkoxyalkyl; haloalkyl substituted with 1, 2, or 3 hydroxy; alkylsulfonylalkyl; —C(O)R¹²; —C(O)NR¹³R^{13a}; alkyl substituted with one or two groups independently aminocarbonyl, alkylaminocarbonyl, and dialkylaminocarbonyl; —C(O)C(O)NR²⁹R^{29a}; —SR¹⁴; —S(O)R¹⁹; —S(O)₂R⁶; —S(O)₂NR⁷R^{7a}; —OR⁹; —NR¹¹R^{11a}; alkyl substituted with one or two —NR⁸R^{8a}; optionally substituted phenyl; optionally substituted phenylalkyl; optionally substituted heteroaryl; optionally substituted heteroarylalkyl; optionally substituted heterocycloalkyl; optionally substituted heterocyloalkylalkyl; optionally substituted cycloalkyl; or optionally substituted cyloalkylalkyl;
R^{5a} and R^{5c} are independently hydrogen, deuterium, or alkyl;
R^{5h} is hydrogen or halo;
R^{5b} is hydrogen, amino, or halo;
R^{5d}, R^{5e}, R^{5f}, and R^{5g} are independently hydrogen or deuterium;
R⁶ is halo; alkyl; alkenyl; alkynyl; haloalkyl; hydroxyalkyl; alkyl substituted with one or two —NR¹⁰R^{10a}; alkyl substituted with one heterocycloalkyloxy; optionally substituted phenyl; optionally substituted phenylalkyl; optionally substituted heterocycloalkyl; optionally substituted heterocycloalkylalkyl; optionally substituted cycloalkyl; or optionally substituted cycloalkylalkyl;
R⁷, R⁸, R¹⁰, R¹¹, R¹³, R¹⁵, R^{15b}, R¹⁶, R²⁹, and R^{29a} are independently hydrogen, alkyl, alkenyl, or alkynyl;
R^{7a} is hydrogen, alkoxy, alkyl, alkenyl, aminoalkyl, alkylaminoalkyl, dialkylaminoalkyl, hydroxyalkyl, alkylsulfonylalkyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, optionally substituted phenyl, or optionally substituted phenylalkyl;
R^{8a} and R^{10a} are independently hydrogen, alkyl, or alkoxycarbonyl;
R⁹ is hydrogen; alkyl; haloalkyl; hydroxyalkyl; optionally substituted phenyl; or alkyl substituted with one or two —NR¹⁰R^{10a};
R^{11a} is hydrogen, alkyl, alkenyl, alkynyl, alkoxycarbonyl, alkylsulfonyl, or optionally substituted phenylsulfonyl;
R¹² is alkyl, alkoxy, or hydroxy;
R^{13a} is hydrogen, alkyl, alkenyl, alkynyl, haloalkyl, optionally substituted cycloalkyl, optionally substituted cycloalkylalkyl, optionally substituted heterocycloalkyl, optionally substituted heterocycloalkylalkyl, optionally substituted phenyl, or optionally substituted phenylalkyl;
R¹⁴ and R¹⁹ are independently alkyl; haloalkyl; or optionally substituted phenyl;
R^{15a} is hydrogen, alkyl, alkenyl, alkynyl, haloalkyl, hydroxyalkyl, aminoalkyl, alkylaminoalkyl, or dialkylaminoalkyl;
R^{16a} is hydrogen, alkyl, alkenyl, alkynyl, haloalkyl, alkoxyalkyl, aminoalkyl, alkylaminoalkyl, dialkylaminoalkyl, optionally substituted heterocycloalkyl, or optionally substituted heterocycloalkylalkyl;
R¹⁷ is alkyl, alkenyl, alkynyl, amino, alkylamino, or dialkylamino;
R¹⁸ is alkyl, hydroxyalkyl, haloalkyl, aminoalkyl, alkylaminoalkyl, or dialkylaminoalkyl;
R²² and R²³ are independently hydrogen, alkyl, alkenyl, alkynyl, alkoxyalkyl, or haloalkyl;
R^{23a} is hydrogen, alkyl, alkenyl, alkynyl, alkoxyalkyl, aminoalkyl, alkylaminoalkyl, dialkylaminoalkyl, hydroxyalkyl, haloalkyl, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl;
R²⁴ is hydrogen, alkyl, aminoalkyl, alkylaminoalkyl, dialkylaminoalkyl, haloalkyl, hydroxyalkyl, or optionally substituted phenylalkyl;
R^{24a} is alkyl, haloalkyl, hydroxyalkyl, aminoalkyl, alkylaminoalkyl, dialkylaminoalkyl, or optionally substituted heterocycloalkyl;
R^{24b} is alkyl, haloalkyl, hydroxyalkyl, aminoalkyl, alkylaminoalkyl, or dialkylaminoalkyl;
R²⁵ is alkyl or haloalkyl;
R²⁶ is alkyl; or optionally substituted heterocycloalkyl;
each R²⁷, when R²⁷ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, aminocarbonyl, alkylaminocarbonyl, dialkylaminocarbonyl, or optionally substituted phenyl; and
R²⁸ is alkyl; haloalkyl; alkoxy; hydroxy; optionally substituted heterocycloalkyl; or optionally substituted phenyl.

In a second aspect, the invention is directed to a pharmaceutical composition which comprises 1) a compound of Formula I or a single stereoisomer or mixture of isomers thereof, optionally as a pharmaceutically acceptable salt or solvate thereof and 2) a pharmaceutically acceptable carrier, excipient, or diluent.

In a third aspect of the invention is a method of inhibiting the in vivo activity of mTOR, the method comprising administering to a patient an effective mTOR-inhibiting amount of a compound of Formula Ia compound of Formula I or a single stereoisomer or mixture of isomers thereof, optionally as a pharmaceutically acceptable salt or solvate thereof or pharmaceutical composition thereof.

In a fourth aspect, the Invention comprises a method for treating a disease, disorder, or syndrome, which method comprises administering to a patient a therapeutically effective amount of a compound of Formula I or a single stereoisomer or mixture of isomers thereof, optionally as a pharmaceutically acceptable salt or solvate thereof, or a pharmaceutical composition comprising a therapeutically effective amount of a compound of Formula I or a single stereoisomer or mixture of isomers thereof, optionally as a pharmaceutically acceptable salt or solvate thereof, and a pharmaceutically acceptable carrier, excipient, or diluent.

In a fifth aspect, the Invention comprises a method for making a Compound of Formula I which method comprises

(a) reacting an intermediate of formula 6c, or a salt thereof:

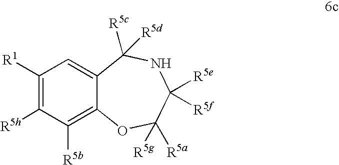

where R¹, R^{5a}, R^{5b}, R^{5c}, R^{5d}, R^{5e}, R^{5f}, R^{5g}, and R^{5h} are as defined in the Summary of the Invention for a Compound of Formula I; with an intermediate of formula R²C(O)X where X is hydroxy or halo, and R² is as defined in the Summary of the Invention for a Compound of Formula Ito yield a Compound of the Invention of Formula I where Z is —C(O)—:

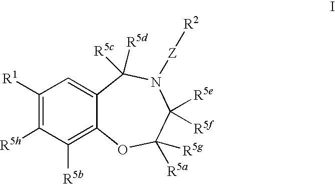

and optionally separating individual isomers; and optionally modifying any of the R¹ and R² groups; and optionally forming a pharmaceutically acceptable salt, hydrate, solvate or combination thereof; or

(b) reacting an intermediate of formula 40, or a salt thereof:

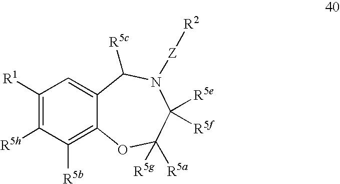

where R is halo or —B(OH)₂, and R^{5a}, R^{5b}, R^{5c}, R^{5d}, R^{5e}, R^{5f}, R^{5g}, and R^{5h} are as defined in the Summary of the Invention for a Compound of Formula I; with an intermediate of formula R¹Y where Y is halo when R is —B(OH)₂ and Y is —B(OH)₂ when R is halo, and R² is as defined in the Summary of the Invention for a Compound of Formula Ito yield a Compound of the Invention of Formula I; and optionally separating individual isomers; and optionally modifying any of the R¹ and R² groups; and optionally forming a pharmaceutically acceptable salt, hydrate, solvate or combination thereof.

### DETAILED DESCRIPTION OF THE INVENTION

### Abbreviations and Definitions

The following abbreviations and terms have the indicated meanings throughout:

| Abbreviation | Meaning |
|---|---|
| AcOH | acetic acid |
| br | broad |
| ° C. | degrees Celsius |
| conc | concentrated |
| d | doublet |
| dd | doublet of doublet |
| dt | doublet of triplet |
| DCM | dichloromethane |
| DIEA or DIPEA | N,N-di-isopropyl-N-ethylamine |
| DMA | N,N-dimethylacetamide |
| DME | 1,2-dimethoxyethane |
| DMEM | Dulbecco's Modification of Eagles Medium |
| DMF | N,N-dimethylformamide |
| DMSO | dimethyl sulfoxide |
| dppf | 1,1′-bis(diphenylphosphano)ferrocene |
| EI | Electron Impact ionization |
| equiv | equivalents |
| FBS | fetal bovine serum |
| g | gram(s) |
| GC/MS | gas chromatography/mass spectrometry |
| h or hr | hour(s) |
| HATU | 2-(1H-7-azabenzotriazol-1-yl)-1,1,3,3-tetramethyl |
| | uronium hexafluorophosphate |
| HPLC | high pressure liquid chromatography |
| L | liter(s) |
| LC/MS | liquid chromatography/mass spectrometry |
| M | molar or molarity |
| m | Multiplet |
| MeOH | methanol |
| mg | milligram(s) |
| MHz | megahertz (frequency) |
| min | minute(s) |
| mL | milliliter(s) |
| μL | microliter(s) |
| μM | micromolar |
| μmol | micromole(s) |
| mM | Millimolar |
| mmol | millimole(s) |
| mol | mole(s) |
| MS | mass spectral analysis |
| Ms | mesyl |
| N | normal or normality |
| NEAA | non-essential amino acids |
| nM | Nanomolar |
| NMR | nuclear magnetic resonance spectroscopy |
| q | Quartet |
| quant | quantitative |
| rt | Room temperature |
| s | Singlet |
| t or tr | Triplet |
| THF | tetrahydrofuran |
| Ts | tosyl |

Deuterium is listed as a specific R^{5a}, R^{5c}, R^{5d}, R^{5e}, R^{5f}, and R^{5g} substituent. Although deuterium is specifically recited in these groups, it does not mean that it and other isotopes of other atoms are excluded from the scope of the invention.

If a group “R” is depicted as “floating” on a ring system, as for example in the formula:

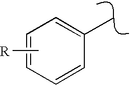

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring, as for example in the formulae:

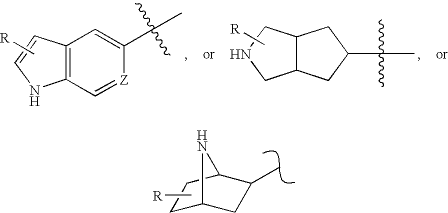

then, unless otherwise defined, a substituent “R” may reside on any atom of the fused ring, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals ═CH—) from one of the ring atoms, so long as a stable structure is formed. In the example depicted, the “R” group may reside on either the 5-membered or the 6-membered ring of the fused ring.

“Acyl” means a —C(O)R radical where R is alkyl, alkenyl, cycloalkyl, cycloalkylalkyl, aryl, aralkyl, heteroaryl, heteroaralkyl, heterocycloalkyl, or heterocycloalkylalkyl, as defined herein, e.g., acetyl, methylcarbonyl, or ethylcarbonyl, and the like.

“Acylamino” means a —NRR′ radical where R is hydrogen, hydroxy, alkyl, or alkoxy and R′ is acyl, as defined herein.

“Acyloxy” means an —OR radical where R is acyl, as defined herein, e.g. cyanomethylcarbonyloxy, and the like.

“Administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., surgery, radiation, and chemotherapy, etc.), “administration” and its variants are each understood to include concurrent and sequential introduction of the compound or prodrug thereof and other agents.

“Alkenyl” means a means a linear hydrocarbon radical of two to six carbon atoms or a branched hydrocarbon radical of three to 6 carbon atoms which radical contains at least one double bond, e.g., ethenyl, propenyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkoxyalkyl” means an alkyl group, as defined herein, substituted with at least one, specifically one, two, or three, alkoxy groups as defined herein. Representative examples include methoxymethyl and the like.

“Alkoxycarbonyl” means a —C(O)R group where R is alkoxy, as defined herein.

“Alkyl” means a linear saturated hydrocarbon radical of one to six carbon atoms or a branched saturated hydrocarbon radical of three to 6 carbon atoms, e.g., methyl, ethyl, propyl, 2-propyl, butyl (including all isomeric forms), or pentyl (including all isomeric forms), and the like.

“Alkylamino” means an —NHR group where R is alkyl, as defined herein.

“Alkylaminoalkyl” means an alkyl group substituted with one or two alkylamino groups, as defined herein.

“Alkylcarbonyl” means a —C(O)R group where R is alkyl, as defined herein.

“Alkylsulfonyl” means an —S(O)₂R group where R is alkyl, as defined herein.

“Alkylsulfonylalkyl” means an alkyl group, as defined herein, substituted with at least one, preferably one or two, alkylsulfonyl groups, as defined herein.

“Alkynyl” means a linear hydrocarbon radical of two to six carbon atoms or a branched hydrocarbon radical of three to 6 carbon atoms which radical contains at least one triple bond, e.g., ethynyl, propynyl, butynyl, pentyn-2-yl and the like.

“Amino” means —NH₂.

“Aminoalkyl” means an alkyl group substituted with at least one, specifically one, two or three, amino groups.

“Aminocarbonyl” means a —C(O)NH₂ group.

“Alkylaminocarbonyl” means a —C(O)NHR group where R is alkyl as defined herein.

“Aryl” means a six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Cyanoalkyl” means an alkyl group, as defined herein, substituted with one or two cyano groups.

“Cycloalkyl” means a monocyclic or fused bicyclic, saturated or partially unsaturated (but not aromatic), hydrocarbon radical of three to ten carbon ring atoms. Fused bicyclic hydrocarbon radical includes bridged ring systems. Unless stated otherwise, the valency of the group may be located on any atom of any ring within the radical, valency rules permitting. One or two ring carbon atoms may be replaced by a —C(O)—, —C(S)—, or —C(═NH)— group. More specifically, the term cycloalkyl includes, but is not limited to, cyclopropyl, cyclobutyl, cyclopentyl, cyclohexyl, cyclohexyl, or cyclohex-3-enyl, and the like.

“Cycloalkylalkyl” means an alkyl group substituted with at least one, specifically one or two, cycloalkyl group(s) as defined herein.

“Dialkylamino” means a —NRR′ radical where R and R′ are alkyl as defined herein, or an N-oxide derivative, or a protected derivative thereof, e.g., dimethylamino, diethylamino, N,N-methylpropylamino or N,N-methylethylamino, and the like.

“Dialkylaminoalkyl” means an alkyl group substituted with one or two dialkylamino groups, as defined herein.

“Dialkylaminocarbonyl” means a —C(O)NRR′ group where R and R′ are alkyl as defined herein.

“Fused ring” means a polycyclic ring system that contains bridged or fused rings; that is, where two rings have more than one shared atom in their ring structures. In this application, fused ring systems are not necessarily all aromatic ring systems. Typically, but not necessarily, fused ring systems share a vicinal set of atoms, for example naphthalene or 1,2,3,4-tetrahydro-naphthalene. A Spiro ring system is not a fused ring system by this definition, but fused ring systems of the invention may themselves have spiro rings attached thereto via a single ring atom of the fused ring system. In some examples, as appreciated by one of ordinary skill in the art, two adjacent groups on an aromatic system may be fused together to form a ring structure. The fused ring structure may contain heteroatoms and may be optionally substituted with one or more groups. It should additionally be noted that saturated carbons of such fused groups (i.e. saturated ring structures) can contain two substitution groups.

“Halogen” or “halo” refers to fluorine, chlorine, bromine and iodine.

“Haloalkoxy” means an —OR′ group where R′ is haloalkyl as defined herein, e.g., trifluoromethoxy or 2,2,2-trifluoroethoxy, and the like.

“Haloalkyl” mean an alkyl group substituted with one or more halogens, specifically 1, 2, 3, 4, 5, or 6 halo atoms, e.g., trifluoromethyl, 2-chloroethyl, 2,2-difluoroethyl, 1,1,1,3,3,3-hexafluoro-propan-2-yl, and the like.

“Heteroaryl” means a monocyclic or fused bicyclic radical of 5 to 14 ring atoms containing one or more, specifically one, two, three, or four ring heteroatoms which are independently —O—, —S(O)ₙ— (n is 0, 1, or 2), —N—, —N(R^{x})—, or N-oxide, with the remaining ring atoms being carbon, wherein the ring comprising a monocyclic radical is aromatic and wherein at least one of the fused rings comprising the bicyclic radical is aromatic. One or two ring carbon atoms of any nonaromatic rings comprising a bicyclic radical may be replaced by a —C(O)—, —C(S)—, or —C(═NH)— group. R^{x} is hydrogen, alkyl, hydroxy, alkoxy, acyl, or alkylsulfonyl. Fused bicyclic radical includes bridged ring systems. Unless stated otherwise, the valency may be located on any atom of any ring of the heteroaryl group, valency rules permitting. When the point of valency is located on the nitrogen, R^{x} is absent. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, 2,3-dihydrobenzofuranyl, benzo[d][1,3]dioxolyl, 2,3-dihydrobenzo[b][1,4]dioxinyl, thiazolyl, isothiazolyl, thiadiazolyl, benzothiazolyl, benzothienyl, and the derivatives thereof, or N-oxide or a protected derivative thereof. The term “5- or 6-membered heteroaryl” describes a subset of the term “heteroaryl.”

“Heteroarylalkyl” means an alkyl group, as defined herein, substituted with at least one, specifically one or two heteroaryl group(s), as defined herein.

“Heterocycloalkyl” means a saturated or partially unsaturated (but not aromatic) monocyclic group of 3 to 8 ring atoms or a saturated or partially unsaturated (but not aromatic) fused bicyclic group of 5 to 12 ring atoms in which one or more, specifically one, two, three, or four ring heteroatoms which are independently O, S(O)ₙ (n is 0, 1, or 2), N, or N(R^{y}) (where R^{y} is hydrogen, alkyl, hydroxy, alkoxy, acyl, or alkylsulfonyl), the remaining ring atoms being carbon. One or two ring carbon atoms may be replaced by a —C(O)—, —C(S)—, or —C(═NH)— group. Fused bicyclic radical includes bridged ring systems. Unless otherwise stated, the valency of the group may be located on any atom of any ring within the radical, valency rules permitting. When the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydrocyclopenta[c]pyrrolyl, octahydroindolyl, octahydroisoindolyl, decahydroisoquinolyl, tetrahydrofuryl, and tetrahydropyranyl, and the derivatives thereof and N-oxide or a protected derivative thereof.

“Heterocycloalkylalkyl” means an alkyl radical, as defined herein, substituted with one or two heterocycloalkyl groups, as defined herein, e.g., morpholinylmethyl, N-pyrrolidinylethyl, and 3-(N-azetidinyl)propyl, and the like.

“Heterocycloalkyloxy” means an —OR group where R is heterocycloalkyl, as defined herein.

“Hydroxyalkyl” means an alkyl group, as defined herein, substituted with at least one, preferably 1, 2, 3, or 4, hydroxy groups.

“Phenylalkyl” means an alkyl group, as defined herein, substituted with one or two phenyl groups.

“Phenylalkyloxy” means an —OR group where R is phenylalkyl, as defined herein.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term. So, for example, in the term “optionally substituted phenylC₁₋₈ alkyl,” optional substitution may occur on both the “C₁₋₈ alkyl” portion and the “phenyl” portion of the molecule may or may not be substituted. A list of exemplary optional substitutions is presented below in the definition of “substituted.”

“Optionally substituted cycloalkyl” means a cycloalkyl group, as defined herein, substituted with one, two, or three groups which groups are independently acyl, acyloxy, acylamino, alkyl, alkenyl, alkoxy, alkenyloxy, alkoxycarbonyl, alkenyloxycarbonyl, alkylthio, alkylsulfinyl, alkylsulfonyl, aminosulfonyl, alkylaminosulfonyl, dialkylaminosulfonyl, alkylsulfonylamino, halo, hydroxy, amino, alkylamino, dialkylamino, aminocarbonyl, alkylaminocarbonyl, dialkylaminocarbonyl, nitro, alkoxyalkyloxy, aminoalkoxy, alkylaminoalkoxy, dialkylaminoalkoxy, carboxy, or cyano. Within the above optional substitutents on “cycloalkyl”, the alkyl and alkenyl, either alone or as part of another substituent on the cycloalkyl ring, are independently optionally substituted with one, two, three, four, or five halo, e.g. haloalkyl, haloalkoxy, haloalkenyloxy, or haloalkylsulfonyl.

“Optionally substituted cycloalkylalkyl” means an alkyl group substituted with at least one, specifically one or two, optionally substituted cycloalkyl groups, as defined herein.

“Optionally substituted heteroaryl” means a heteroaryl group optionally substituted with one, two, or three substituents which substituents are independently acyl, acylamino, acyloxy, alkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, alkylaminoalkoxy, or dialkylaminoalkoxy. Within the optional substituents on “heteroaryl”, the alkyl and alkenyl, either alone or as part of another group (including, for example, the alkyl in alkoxycarbonyl), are independently optionally substituted with one, two, three, four, or five halo.

“Optionally substituted heteroarylalkyl” means an alkyl group, as defined herein, substituted with at least one, specifically one or two, optionally substituted heteroaryl group(s), as defined herein.

“Optionally substituted heterocycloalkyl” means a heterocycloalkyl group, as defined herein, optionally substituted with one, two, or three substituents which substituents are independently acyl, acylamino, acyloxy, alkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or phenylalkyl. Within the optional substituents on “heterocycloalkyl”, the alkyl and alkenyl, either alone or as part of another group (including, for example, the alkyl in alkoxycarbonyl), are independently optionally substituted with one, two, three, four, or five halo.

“Optionally substituted heterocycloalkylalkyl” means an alkyl group, as defined herein, substituted with at least one, specifically one or two, optionally substituted heterocycloalkyl group(s) as defined herein.

“Optionally substituted phenyl” means a phenyl group optionally substituted with one, two, or three substituents where the substituents are independently acyl, acylamino, acyloxy, alkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, or aminoalkoxy. Within the optional substituents on “phenyl”, the alkyl and alkenyl, either alone or as part of another group (including, for example, the alkyl in alkoxycarbonyl), are independently optionally substituted with one, two, three, four, or five halo.

“Optionally substituted phenylalkyl” means an alkyl group, as defined herein, substituted with one or two optionally substituted phenyl groups, as defined herein.

“Optionally substituted phenylsulfonyl” means an —S(O)₂R group where R is optionally substituted phenyl, as defined herein.

“Oxo” means an oxygen which is attached via a double bond.

“Yield” for each of the reactions described herein is expressed as a percentage of the theoretical yield.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

“Patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Treating” or “treatment” of a disease, disorder, or syndrome, as used herein, includes (i) preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

### EMBODIMENTS OF THE INVENTION

The following paragraphs present a number of embodiments of compounds of the invention. In each instance the embodiment includes both the recited compounds, as well as a single stereoisomer or mixture of stereoisomers thereof, as well as a pharmaceutically acceptable salt thereof.

### Embodiment (1)

The invention is directed to a Compound of Formula I where
Z is —C(O)—;
R¹ is phenyl optionally substituted with one, two, or three R²⁰ where each R²⁰ is independently nitro; cyano; halo; alkyl; alkenyl; alkynyl; haloalkyl; —NR¹⁵R^{15a}; —NR¹⁵C(O)R¹⁸; —NR¹⁵S(O)₂R¹⁸; —NR¹⁵C(O)NR^{15a}R^{15b}; —OR⁹; —C(O)OR⁹; —C(O)R²⁶; —C(O)NR¹⁶R^{16a}; alkyl substituted with one or two —C(O)NR¹⁶R^{16a}; S(O)₂R¹⁷; heteroaryl optionally substituted with 1, 2, or 3 R²⁷; or heterocycloalkyl optionally substituted with one or two groups which groups are independently alkyl, oxo, alkoxycarbonyl, or phenylalkyl; or
R¹ is heteroaryl or an N-oxide thereof, optionally substituted with one, two, or three R²¹; wherein each R²¹ is independently oxo; cyano; alkyl; alkenyl; alkynyl; halo; haloalkyl; hydroxyalkyl; alkoxy; alkoxyalkyl; optionally substituted cycloalkyl; optionally substituted cycloalkylalkyl; optionally substituted heterocycloalkyl; optionally substituted heterocycloalkylalkyl; optionally substituted heteroaryl; optionally substituted heteroarylalkyl; alkyl substituted with phenylalkyloxy; —OR²⁴; —SR²⁵; —S(O)R²⁵; —S(O)₂R²⁵; —C(O)OR²²; —C(O)NR²³R^{23a}; —C(O)R^{24a}; —NR²³R^{23a}; alkyl substituted with one —NR²³R^{23a}; —NR²³C(O)OR^{24a}; —NR²³C(O)R^{23a}; alkyl substituted with one —NR²³C(O)R^{24a}; —NR²³C(O)NR^{23a}R²⁴; —NR²³C(═NH)NR^{23a}R²⁴; or —NR²³S(O)₂R^{23a};
R² is phenyl or naphthyl, each of which is substituted with R^{3a}, R^{3b}, R^{3c}, and R^{3d}; R² is HET¹ optionally substituted with R^{4a}, R^{4b}, and R^{4c}; or R² is HET² optionally substituted with R^{4a}, R^{4b}, R^{4c}, and R^{4d};
HET¹ is a 5- or 6-membered heteroaryl where the ring atom to which Z is attached is a carbon atom;
HET² is an 8- to 14-membered fused bicyclic ring containing one, two, three, or four ring heteroatoms which are independently O, S, S(O), S(O)₂, or N, with the remaining ring atoms being carbon, where the ring atom attached to Z is carbon and where the ring attached to Z is aromatic and the other ring of HET² is partially or fully unsaturated;
R^{3a}, R^{3b}, R^{3c}, and R^{3d} are independently hydrogen; nitro; cyano; halo; alkyl; alkenyl; alkynyl; cyanoalkyl; haloalkyl; hydroxyalkyl; alkoxyalkyl; haloalkyl substituted with 1, 2, or 3 hydroxy; alkylsulfonylalkyl; —C(O)R²⁸; —C(O)NR¹³R^{13a}; —C(O)C(O)NR²⁹R^{29a}; —SR¹⁴; —S(O)R¹⁹; —S(O)₂R⁶; —S(O)₂NR⁷R^{7a}; —OR⁹; —NR¹¹R^{11a}; alkyl substituted with one —NR⁸R^{8a}; phenyl optionally substituted with alkylsulfonyl; optionally substituted phenylalkyl; heteroaryl optionally substituted with 1, 2, or 3 groups which groups are independently alkyl or haloalkyl; optionally substituted heteroarylalkyl; heterocycloalkyl optionally substituted with 1, 2, or 3 groups which groups are independently alkyl or alkoxycarbonyl; or optionally substituted cycloalkyl;
R^{4a}, R^{4b}, R^{4c}, and R^{4d} are independently nitro; cyano; halo; oxo, alkyl; alkenyl; alkynyl; cyanoalkyl; haloalkyl; hydroxyalkyl; alkoxyalkyl; haloalkyl substituted with 1, 2, or 3 hydroxy; alkylsulfonylalkyl; —C(O)R¹²; —C(O)NR¹³R^{13a}; alkyl substituted with one aminocarbonyl, alkylaminocarbonyl, or dialkylaminocarbonyl; —C(O)C(O)NR²⁹R^{29a}; —SR¹⁴; —S(O)R¹⁹; —S(O)₂R⁶; —S(O)₂NR⁷R^{7a}; —OR⁹; —NR¹¹R^{11a}; alkyl substituted with one —NR⁸R^{8a}; phenyl optionally substituted with 1, 2, or 3 groups which groups are independently halo, alkyl, alkoxy, and alkylsulfonyl; optionally substituted phenylalkyl; heteroaryl optionally substituted with 1, 2, or 3 groups which groups are independently alkyl or haloalkyl; optionally substituted heteroarylalkyl; heterocycloalkyl optionally substituted with 1, 2, or 3 groups which groups are independently alkyl or alkoxycarbonyl; or optionally substituted cycloalkyl;
R^{5a} and R^{5c} are independently hydrogen, deuterium, or alkyl;
R^{5h} is hydrogen or halo;
R^{5b} is hydrogen, amino, or halo;
R^{5d}, R^{5e}, R^{5f}, and R^{5g} are independently hydrogen or deuterium;
R⁶ is halo; alkyl; alkenyl; alkynyl; haloalkyl; hydroxyalkyl; alkyl substituted with one —NR¹⁰R^{10a}; alkyl substituted with heterocycloalkyloxy; phenyl optionally substituted with 1, 2 or 3 groups which groups are independently halo, alkyl, amino, alkylamino, dialkylamino, and alkoxy; heterocycloalkyl optionally substituted with 1 or 2 groups which groups are independently alkyl and alkoxycarbonyl; optionally substituted heterocycloalkylalkyl; optionally substituted cycloalkyl; or optionally substituted cycloalkylalkyl;
R⁷, R⁸, R¹⁰, R¹¹, R¹³, R¹⁵; R^{15b}; R¹⁶; R²⁹; and R^{29a} are independently hydrogen, alkyl, alkenyl, or alkynyl;
R^{7a} is hydrogen, alkoxy, alkyl, alkenyl, aminoalkyl, alkylaminoalkyl, dialkylaminoalkyl, hydroxyalkyl, alkylsulfonylalkyl, optionally substituted cycloalkyl, optionally substituted heterocycloalkylalkyl, optionally substituted heteroarylalkyl, or optionally substituted phenyl;
R^{8a} and R^{10a} are independently hydrogen, alkyl, or alkoxycarbonyl;
R⁹ is hydrogen; alkyl; haloalkyl; hydroxyalkyl; optionally substituted phenyl; or alkyl substituted with one —NR¹⁰R^{10a};
R^{11a} is hydrogen, alkyl, alkenyl, alkynyl, alkoxycarbonyl, alkylsulfonyl, or optionally substituted phenylsulfonyl;
R¹² is alkyl, alkoxy, or hydroxy;
R^{13a} is hydrogen, alkyl, alkenyl, alkynyl, haloalkyl, optionally substituted cycloalkyl, optionally substituted cycloalkylalkyl, optionally substituted heterocycloalkylalkyl, optionally substituted phenyl, or optionally substituted phenylalkyl;
R¹⁴ and R¹⁹ are independently alkyl; haloalkyl; or phenyl optionally substituted with 1, 2, or 3 alkyl;
R^{15a} is hydrogen, alkyl, alkenyl, alkynyl, haloalkyl, hydroxyalkyl, aminoalkyl, alkylaminoalkyl, or dialkylaminoalkyl;
R^{16a} is hydrogen, alkyl, alkenyl, alkynyl, haloalkyl, alkoxyalkyl, aminoalkyl, alkylaminoalkyl, dialkylaminoalkyl, heterocycloalkyl optionally substituted with one alkyl, or optionally substituted heterocycloalkylalkyl;
R¹⁷ is alkyl, alkenyl, alkynyl, amino, alkylamino, or dialkylamino;
R¹⁸ is alkyl, hydroxyalkyl, haloalkyl, aminoalkyl, alkylaminoalkyl, or dialkylaminoalkyl;
R²² and R²³ are independently hydrogen, alkyl, alkenyl, alkynyl, alkoxyalkyl, or haloalkyl;
R^{23a} is hydrogen, alkyl, alkenyl, alkynyl, alkoxyalkyl, aminoalkyl, alkylaminoalkyl, dialkylaminoalkyl, hydroxyalkyl, haloalkyl, cycloalkyl optionally substituted with one or two groups which groups are independently amino, alkylamino, or dialkylamino, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl;
R²⁴ is hydrogen, alkyl, aminoalkyl, alkylaminoalkyl, dialkylaminoalkyl, haloalkyl, hydroxyalkyl, or optionally substituted phenylalkyl;
R^{24a} is alkyl, haloalkyl, hydroxyalkyl, aminoalkyl, alkylaminoalkyl, or dialkylaminoalkyl;
R²⁵ is alkyl or haloalkyl;
R²⁶ is alkyl; or heterocycloalkyl optionally substituted with alkyl or alkoxycarbonyl;
each R²⁷, when R²⁷ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, aminocarbonyl, alkylaminocarbonyl, dialkylaminocarbonyl, or optionally substituted phenyl; and
R²⁸ is alkyl; haloalkyl; alkoxy; hydroxy; heterocycloalkyl optionally substituted with 1 or 2 groups which groups are independently alkyl or alkoxycarbonyl; or optionally substituted phenyl.

### Embodiment (A1)

In one embodiment, the Compound of Formula I is that where R^{5c} and R^{5d} are deuterium and R^{5a}, R^{5e}, R^{5f}, and R^{5g} are hydrogen; and all other groups are as defined in the Summary of the Invention for a Compound of Formula I or as defined in embodiment (1).

### Embodiment (A2)

In another embodiment, the Compound of Formula I is that where R^{5a}, R^{5c}, R^{5d}, R^{5e}, R^{5f}, and R^{5g} are deuterium; and all other groups are as defined in the Summary of the Invention for a Compound of Formula I or as defined in embodiment (1).

### Embodiment (A3)

In another embodiment, the Compound of Formula I is that where R^{5e} and R^{5f} are deuterium and R^{5a}, R^{5c}, R^{5d}, and R^{5g} are hydrogen; and all other groups are as defined in the Summary of the Invention for a Compound of Formula I or as defined in embodiment (1).

### Embodiment (A4)

In another embodiment, the Compound of Formula I is that where R^{5a} and R^{5g} are deuterium and R^{5e}, R^{5c}, R^{5d}, and R^{5f} are hydrogen; and all other groups are as defined in the Summary of the Invention for a Compound of Formula I or as defined in embodiment (1).

### Embodiments (A5)

In another embodiment, the Compound of Formula I is that where R^{5a} is hydrogen or alkyl and R^{5c}, R^{5d}, R^{5e}, R^{5f}, and R^{5g} are hydrogen; and all other groups are as defined in the Summary of the Invention for a Compound of Formula I. In another embodiment, the Compound of Formula I is that where R^{5a} is alkyl and R^{5c}, R^{5d}, R^{5e}, R^{5f}, and R^{5g} are hydrogen; and all other groups are as defined in the Summary of the Invention for a Compound of Formula I or as defined in embodiment (1).

### Embodiments (A6)

In another embodiment, the Compound of Formula I is that where R^{5b} is hydrogen, halo, or amino and R^{5a}, R^{5c}, R^{5d}, R^{5e}, R^{5f}, R^{5g}, and R^{5h} are hydrogen; and all other groups are as defined in the Summary of the Invention for a Compound of Formula I or as defined in embodiment (1). In another embodiment, the Compound of Formula I is that where R^{5b} is halo and R^{5a}, R^{5c}, R^{5d}, R^{5e}, R^{5f}, R^{5g}, and R^{5h} are hydrogen; and all other groups are as defined in the Summary of the Invention for a Compound of Formula I or as defined in embodiment (1). In another embodiment, the Compound of Formula I is that where R^{5b} is fluoro and R^{5a}, R^{5c}, R^{5d}, R^{5e}, R^{5f}, R^{5g}, and R^{5h} are hydrogen; and all other groups are as defined in the Summary of the Invention for a Compound of Formula I or as defined in embodiment (1). In another embodiment, the Compound of Formula I is that where R^{5b} is amino; R^{5a}, R^{5c}, R^{5d}, R^{5e}, R^{5f}, R^{5g}, and R^{5h} are hydrogen; and all other groups are as defined in the Summary of the Invention for a Compound of Formula I or as defined in embodiment (1).

### Embodiments (A7)

In another embodiment, the Compound of Formula I is that where R^{5h} is hydrogen or halo and R^{5a}, R^{5c}, R^{5d}, R^{5e}, R^{5f}, R^{5g}, and R^{5b} are hydrogen; and all other groups are as defined in the Summary of the Invention for a Compound of Formula I or as defined in embodiment (1). In another embodiment, the Compound of Formula I is that where R^{5h} is halo and R^{5a}, R^{5c}, R^{5d}, R^{5e}, R^{5f}, R^{5g}, and R^{5b} are hydrogen; and all other groups are as defined in the Summary of the Invention for a Compound of Formula I or as defined in embodiment (1). In another embodiment, the Compound of Formula I is that where R^{5h} is fluoro and R^{5a}, R^{5c}, R^{5d}, R^{5e}, R^{5f}, R^{5g}, and R^{5b} are hydrogen; and all other groups are as defined in the Summary of the Invention for a Compound of Formula I or as defined in embodiment (1).

Another embodiment of the Invention is directed to a Compound of Formula III

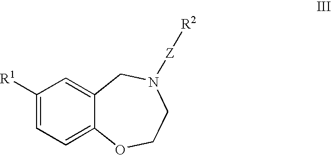

where all other groups are as defined in the Summary of the Invention for a Compound of Formula I or as defined in embodiment (1).

### Embodiment (B)

In another embodiment, the Compound of Formula I is according to Formula I(d)

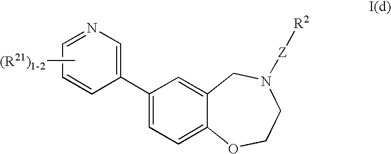

or an N-oxide thereof, where Z, R² and R²¹ are as defined in the Summary of the Invention for a Compound of Formula I or as defined in embodiment (1).

### Embodiment (B1)

In another embodiment, the Compound of Formula I is according to Formula I(d), or an N-oxide thereof, where
one R²¹ is hydroxyalkyl; optionally substituted heteroaryl; —C(O)OR²²; —NR²³R^{23a}; —OR²⁴; —NR²³C(O)R^{23a}; —C(O)NR²³R^{23a}; —NR²³C(O)NR^{23a}R²⁴; —NR²³C(═NH)NR^{23a}R²⁴; or —NR²³S(O)₂R^{23a}; and
the second R²¹, when present, is halo, alkyl, cyano, —OR²⁴, or —C(O)NR²³R^{23a};
Z, R² and all other groups are as defined in the Summary of the Invention for a Compound of Formula I or as defined in embodiment (1).

### Embodiment (B2)

In another embodiment, the Compound is according to Formula I(d), or an N-oxide thereof, where
one R²¹ is hydroxyalkyl; —C(O)OR²²; —NR²³R^{23a}; —OR²⁴; —NR²³C(O)R^{23a}; —C(O)NR²³R^{23a}; —NR²³C(O)NR^{23a}R²⁴; NR²³C(═NH)NR^{23a}R²⁴; —NR²³S(O)₂R^{23a}; or heteroaryl optionally substituted with halo;
the second R²¹, when present, is halo, alkyl, cyano, hydroxy or —C(O)NR²³R^{23a};
R²² is alkyl;
R²³ is hydrogen or alkyl;
R^{23a} is hydrogen, alkyl, aminoalkyl, alkylaminoalkyl, dialkylaminoalkyl, hydroxyalkyl, alkoxyalkyl, cycloalkyl optionally with amino, optionally substituted heterocycloalkylalkyl, optionally substituted heteroaryl;
R²⁴ is hydrogen, alkyl, or optionally substituted phenylalkyl; and
Z, R² and all other groups are as defined in the Summary of the Invention for a Compound of Formula I or as defined in embodiment (1).

### Embodiment (B3)

In another embodiment, the Compound is according to Formula I(d), or an N-oxide thereof, where
one R²¹ is methoxy; hydroxymethyl; imidazolyl; benzimidazolyl; benzimidazolyl substituted with fluoro; methoxycarbonyl; —NH₂; N-methylamino; N,N-dimethylamino; N-ethylamino; N-(n-propyl)-amino; N-(isopropyl)-amino; N-(2-hydroxyethyl)-amino; N-(2-methoxyethyl)-amino; 2-(N,N-dimethylamino)-ethylamino; 2-(N-methylamino)-ethylamino; 3-(N,N-dimethylamino)-propylamino; N-cyclopentylamino; 1-amino-cyclobut-1-ylcarbonylamino; 1-amino-cycloprop-1-ylcarbonylamino; N-(pyrrolidin-1-ylmethyl)-amino; N-(pyrrolidin-2-ylmethyl)-amino; N-(pyrrolidin-3-ylmethyl)-amino; N-(2-(pyrrolidin-1-yl)-ethyl)-amino; 2-(morpholin-4-yl)-ethylamino; pyrimidin-2-ylamino; pyrimidin-4-ylamino; pyrimidin-5-ylamino; benzyloxy; —NHC(O)H; —NHC(O)CH₂NHCH₃; —NHC(O)CH₂N(CH₃)₂; —NHC(O)CH₂NHCH₂CH₃; —NHC(O)CH₂CH₂NHCH₃; —NHC(O)CH₂NH₂; —NHC(O)CH₂CH₂NH₂; —NHC(O)CH(CH₃)(NH₂); —NHC(O)CH(CH₃)(NHCH₃); —C(O)NHCH₃; —C(O)NH(CH₂CH₃); —C(O)N(CH₃)₂; —NHC(O)NH(CH₂CH₃); or —NHC(═NH)NH₂; —NHS(O)₂CH₃; and
the second R²¹, when present, is chloro, fluoro, methyl, cyano, hydroxy, methoxy, benzyloxy, —C(O)NHCH₃, or —C(O)N(CH₃)₂;
Z, R² and all other groups are as defined in the Summary of the Invention for a Compound of Formula I or as defined in embodiment (1).

### Embodiments (B4)

In another embodiment, the Compound is according to Formula I(d), or an N-oxide thereof, where one R²¹ is —C(O)OR²²; —NR²³R^{23a}; —OR²⁴; —NR²³C(O)R^{23a}; —C(O)NR²³R^{23a}; —NR²³C(O)NR^{23a}R^{24a}; —NR²³C(═NH)NR^{23a}R²⁴; or heteroaryl optionally substituted with halo; the second R²¹, when present, is halo, alkyl, hydroxy, —C(O)NHCH₃, or —C(O)N(CH₃)₂; and Z, R² and all other groups are as defined in the Summary of the Invention for a Compound of Formula I or as defined in embodiment (1). In another embodiment, the Compound is according to Formula I(d), or an N-oxide thereof, where one R²¹ is —C(O)OR²²; —NR²³R^{23a}; —NR²³C(O)R^{23a}; —NR²³C(O)NR^{23a}R^{24a}; —NR²³C(═NH)NR^{23a}R²⁴; or heteroaryl optionally substituted with halo; the second R²¹, when present, is chloro, fluoro, methyl, hydroxy, —C(O)NHCH₃, or —C(O)N(CH₃)₂; and Z, R² and all other groups are as defined in the Summary of the Invention for a Compound of Formula I or as defined in embodiment (1). In another embodiment, the Compound is according to Formula I(d), or an N-oxide thereof, where one R²¹ is imidazolyl; benzimidazolyl substituted with fluoro; methoxycarbonyl; —NH₂; N-methylamino; N,N-dimethylamino; N-ethylamino; N-(n-propyl)-amino; N-(isopropyl)-amino; N-(2-hydroxyethyl)-amino; N-(2-methoxyethyl)-amino; 2-(N,N-dimethylamino)-ethylamino; 2-(N-methylamino)-ethylamino; N-cyclopentylamino; 1-amino-cyclobut-1-ylcarbonylamino; N-(pyrrolidin-2-ylmethyl)-amino; N-(2-(pyrrolidin-1-yl)-ethyl)-amino; —NHC(O)CH₂NHCH₃; —NHC(O)CH₂NHCH₂CH₃; —NHC(O)CH₂CH₂NHCH₃; —NHC(O)CH₂NH₂; —NHC(O)CH₂CH₂NH₂; —NHC(O)CH(CH₃)(NH₂); —NHC(O)NH(CH₂CH₃); or —NHC(═NH)NH₂; the second R²¹, when present, is chloro, fluoro, methyl, hydroxy, —C(O)NHCH₃, or —C(O)N(CH₃)₂; and Z, R² and all other groups are as defined in the Summary of the Invention for a Compound of Formula I or as defined in embodiment (1). In another embodiment, the Compound is according to Formula I(d), or an N-oxide thereof, where one R²¹ is imidazolyl; benzimidazolyl substituted with fluoro; methoxycarbonyl; —NH₂; N-methylamino; N,N-dimethylamino; N-ethylamino; N-(n-propyl)-amino; N-(isopropyl)-amino; N-(2-hydroxyethyl)-amino; N-(2-methoxyethyl)-amino; 2-(N,N-dimethylamino)-ethylamino; 2-(N-methylamino)-ethylamino; N-cyclopentylamino; 1-amino-cyclobut-1-ylcarbonylamino; N-(pyrrolidin-2-ylmethyl)-amino; N-(2-(pyrrolidin-1-yl)-ethyl)-amino; —NHC(O)CH₂NHCH₃; —NHC(O)CH₂NHCH₂CH₃; —NHC(O)CH₂CH₂NHCH₃; —NHC(O)CH₂NH₂; —NHC(O)CH₂CH₂NH₂; —NHC(O)CH(CH₃)(NH₂); —NHC(O)NH(CH₂CH₃); or —NHC(═NH)NH₂; the second R²¹, when present is fluoro; and R² and all other groups are as defined in the Summary of the Invention for a Compound of Formula I or as defined in embodiment (1). In another embodiment, the Compound is according to Formula I(d), or an N-oxide thereof, where one R²¹ is —NR²³R^{23a}, heteroaryl, —NR²³C(═NH)NR^{23a}R²⁴, —NR²³C(O)R^{24a}, —C(O)OR²², —OR²⁴, or —C(O)NR²³R^{23a}; the second R²¹ is not present; and R² and all other groups are as defined in the Summary of the Invention for a Compound of Formula I or as defined in embodiment (1). In another embodiment, the Compound is according to Formula I(d), or an N-oxide thereof, where one R²¹ is imidazolyl; benzimidazolyl substituted with fluoro; —NH₂; N-methylamino; N,N-dimethylamino; N-ethylamino; N-(n-propyl)-amino; N-(isopropyl)-amino; N-(2-hydroxyethyl)-amino; N-(2-methoxyethyl)-amino; 2-(N-methylamino)-ethylamino; N-cyclopentylamino; —NHC(O)CH₂NHCH₃; —NHC(O)CH₂NHCH₂CH₃; —NHC(O)CH₂CH₂NHCH₃; —NHC(O)CH₂NH₂; —NHC(O)CH₂CH₂NH₂; —NHC(O)CH(CH₃)(NH₂); or —NHC(═NH)NH₂; the second R²¹ is not present; and R² and all other groups are as defined in the Summary of the Invention for a Compound of Formula I or as defined in embodiment (1).

### Embodiment (C)

In another embodiment, the Compound of Formula I or Formula III is that where R¹ is a 5-membered heteroaryl or an N-oxide thereof, optionally substituted with one, two, or three R²¹ groups where R²¹ and all other groups are as defined in the Summary of the Invention for a Compound of Formula I or as defined in embodiment (1).

### Embodiment (C1)

In another embodiment, the Compound of Formula I or Formula III is that where
R¹ is a 5-membered heteroaryl or an N-oxide thereof, optionally substituted with one or two R²¹; wherein each R²¹ is independently oxo, alkyl; halo; cyano; haloalkyl; alkoxy; alkoxyalkyl; hydroxyalkyl; optionally substituted cycloalkyl; optionally substituted cycloalkylalkyl; optionally substituted heterocycloalkyl; optionally substituted heterocycloalkylalkyl; optionally substituted heteroaryl; optionally substituted heteroarylalkyl; —C(O)OR²²; —NR²³R^{23a}; alkyl substituted with one —NR²³R^{23a}; —OR²⁴; —SR²⁵; —S(O)R²⁵; —S(O)₂R²⁵; —NR²³C(O)OR^{24a}; —NR²³C(O)R^{23a}; alkyl substituted with one —NR²³C(O)R^{24a}; alkyl substituted with phenylalkyloxy; —C(O)NR²³R^{23a}; —C(O)R^{24a}; —NR²³C(O)NR^{23a}R²⁴; —NR²³C(═NH)NR^{23a}R²⁴; or —NR²³S(O)₂R^{23a};
R² and all other groups are as defined in the Summary of the Invention for a Compound of Formula I or as defined in embodiment (1).

### Embodiment (C2)

In another embodiment, the Compound of Formula I or Formula III is that where R¹ is a 5-membered heteroaryl optionally substituted with one or two R²¹ where each R²¹ is independently alkyl; optionally substituted heteroaryl; —C(O)OR²²; —NR²³R^{23a}; alkyl substituted with one —NR²³R^{23a}; —NR²³C(O)R^{24a}; or —C(O)NR²³R^{23a}; R² and all other groups are as defined in the Summary of the Invention for a Compound of Formula I or as defined in embodiment (1).

### Embodiments (C3)

In another embodiment, the Compound of Formula I or Formula III is that where R¹ is a 5-membered heteroaryl optionally substituted with one R²¹ wherein R²¹ is alkyl; imidazolyl; —C(O)OR²²; —NR²³R^{23a}; alkyl substituted with one —NR²³R^{23a}; —NR²³C(O)R^{24a}; or —C(O)NR²³R^{23a}; and where R¹ is additionally optionally substituted with a second R²¹ wherein the second R²¹ is alkyl; and where R²² is alkyl; each R²³ is hydrogen; each R^{23a} is independently hydrogen, alkyl, cycloalkyl optionally substituted with one amino or one alkylamino, optionally substituted heterocycloalkylalkyl, aminoalkyl, alkylaminoalkyl, optionally substituted heteroaryl; R^{24a} is aminoalkyl; and R² and all other groups are as defined in the Summary of the Invention for a Compound of Formula I or as defined in embodiment (1). In another embodiment, the Compound of Formula I or Formula III is that where R¹ is a 5-membered heteroaryl optionally substituted with one R²¹ where R²¹ is methyl; imidazol-2-yl; methoxycarbonyl; tert-butoxycarbonyl; —NH₂; —NHCH₂CH₂NHCH₃; —NHC(O)CH₃; —NHC(O)CH₂NH₂; —NHC(O)CH₂NHCH₃; —NHC(O)CH(CH₃)(NH₂); —NHC(O)CH(CH₃)(NHCH₃); 1-amino-cycloprop-1-ylcarbonylamino; 1-(methylamino)-cycloprop-1-ylcarbonylamino; 1-amino-cyclobut-1-ylcarbonylamino; pyrazolylamino; pyrrolidin-1-ylmethylamino; pyrrolidin-2-ylmethylamino; pyrrolidin-3-ylmethylamino; or —C(O)NHCH₃; and where R¹ is additionally optionally substituted with a second R²¹ where the second R²¹, if present, is methyl; and R² and all other groups are as defined in the Summary of the Invention for a Compound of Formula I or as defined in embodiment (1).

### Embodiment (C4)

In another embodiment, the Compound of Formula I or Formula III is that where
R¹ is pyrazolyl, thiazolyl, thienyl, oxazolyl, or thiadiazolyl, each of which is optionally substituted with one or two R²¹ where each R²¹ is independently oxo, alkyl; halo; cyano; haloalkyl; alkoxy; alkoxyalkyl; hydroxyalkyl; optionally substituted cycloalkyl; optionally substituted cycloalkylalkyl; optionally substituted heterocycloalkyl; optionally substituted heterocycloalkylalkyl; optionally substituted heteroaryl; optionally substituted heteroarylalkyl; —C(O)OR²²; —NR²³R^{23a}; alkyl substituted with one —NR²³R^{23a}; —OR²⁴; —SR²⁵; —S(O)R²⁵; —S(O)₂R²⁵; —NR²³C(O)OR^{24a}; —NR²³C(O)R^{23a}; alkyl substituted with one —NR²³C(O)R^{24a}; alkyl substituted with phenylalkyloxy; —C(O)NR²³R^{23a}; —C(O)R^{24a}; —NR²³C(O)NR^{23a}R²⁴; —NR²³C(═NH)NR^{23a}R²⁴; or —NR²³S(O)₂R^{23a};
R² and all other groups are as defined in the Summary of the Invention for a Compound of Formula I or as defined in embodiment (1).

### Embodiment (C5)

In another embodiment, the Compound of Formula I or Formula III is that where
R¹ is pyrazolyl, thiazolyl, thienyl, oxazolyl, or thiadiazolyl, each of which is optionally substituted with one or two R²¹ wherein each R²¹ is alkyl; optionally substituted heteroaryl; —C(O)OR²²; —NR²³R^{23a}; alkyl substituted with one —NR²³R^{23a}; —NR²³C(O)R^{24a}; or —C(O)NR²³R^{23a};
R² and all other groups are as defined in the Summary of the Invention for a Compound of Formula I or as defined in embodiment (1).

### Embodiments (C6)

In another embodiment, the Compound of Formula I or Formula III is that where R¹ is pyrazolyl, thiazolyl, thienyl, oxazolyl, or thiadiazolyl, each of which is optionally substituted with one R²¹ wherein R²¹ is alkyl; imidazolyl; —C(O)OR²²; —NR²³R^{23a}; alkyl substituted with one —NR²³R^{23a}; —NR²³C(O)R^{24a}; or —C(O)NR²³R^{23a}; and where R¹ is additionally optionally substituted with a second R²¹ wherein the second R²¹ is which is alkyl; and where R²² is alkyl; R²³ is hydrogen; each R^{23a} is independently hydrogen, alkyl, optionally substituted cycloalkyl, optionally substituted heterocycloalkylalkyl, aminoalkyl, alkylaminoalkyl, optionally substituted heteroaryl; R^{24a} is aminoalkyl; and R² and all other groups are as defined in the Summary of the Invention for a Compound of Formula I or as defined in embodiment (1). In another embodiment, the Compound of Formula I or Formula III is that where R¹ is pyrazolyl, thiazolyl, thienyl, oxazolyl, or thiadiazolyl, each of which is optionally substituted with one R²¹ wherein R²¹ is methyl; imidazol-2-yl; methoxycarbonyl; tert-butoxycarbonyl; —NH₂; —NHCH₂CH₂NHCH₃; —NHC(O)CH₃; —NHC(O)CH₂NH₂; —NHC(O)CH₂NHCH₃; —NHC(O)CH(CH₃)(NH₂); —NHC(O)CH(CH₃)(NHCH₃); 1-amino-cycloprop-1-ylcarbonylamino; 1-(methylamino)-cycloprop-1-ylcarbonylamino; 1-amino-cyclobut-1-ylcarbonylamino; pyrazolylamino; pyrrolidin-1-ylmethylamino; pyrrolidin-2-ylmethylamino; pyrrolidin-3-ylmethylamino; or —C(O)NHCH₃; and where R¹ is additionally optionally substituted with a second R²¹ where the second R²¹ is methyl; and R² and all other groups are as defined in the Summary of the Invention for a Compound of Formula I or as defined in embodiment (1).

### Embodiments (C7)

In another embodiment, the Compound of Formula I or Formula III is that where R¹ is thiazol-5-yl or 1,3,4-thiadiazol-2-yl, where R¹ is optionally substituted with one R²¹ wherein R²¹ is alkyl; imidazolyl; —C(O)OR²²; —NR²³R^{23a}; alkyl substituted with one —NR²³R^{23a}; —NR²³C(O)R^{24a}; or —C(O)NR²³R^{23a}; and R² and all other groups are as defined in the Summary of the Invention for a Compound of Formula I or as defined in embodiment (1). In another embodiment, the Compound of Formula I or Formula III is that wherein R¹ is thiazol-5-yl or 1,3,4-thiadiazol-2-yl, where R¹ is optionally substituted with one R²¹ wherein R²¹ is —NH₂, —NHCH₂CH₂NHCH₃, pyrrolidin-2-ylmethylamino, pyrazolylamino, —NHC(O)CH₃, —NHC(O)CH(CH₃)(NH₂), —NHC(O)CH₂NHCH₃, —NHC(O)CH₂NH₂, 1-amino-cycloprop-1-ylcarbonylamino, or 1-amino-cyclobut-1-ylcarbonylamino; and R² and all other groups are as defined in the Summary of the Invention for a Compound of Formula I or as defined in embodiment (1).

### Embodiment (D)

In another embodiment, the Compound of Formula I is according to Formula I(e1) or I(e2):

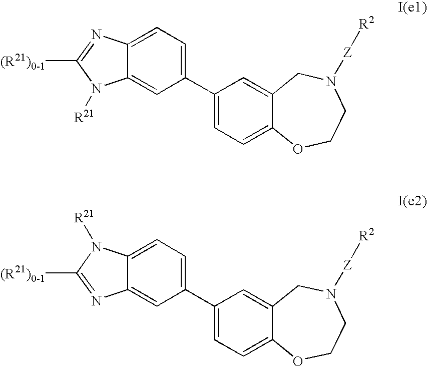

where Z is —C(O)—, and each R²¹ (independently of each other) and R² are as defined in the Summary of the Invention for a Compound of Formula I or as defined in embodiment (1).

### Embodiments (D1)

In another embodiment, the Compound of Formula I is according to Formula I(e1) or I(e2) where the R²¹ at the 1-position is alkyl, hydroxyalkyl, or alkyl substituted with one —NR²³R^{23a} and the R²¹ at the 2-position, when present, is alkyl or alkyl substituted with one —NR²³R^{23a}; and all other groups are as defined in the Summary of the Invention for a Compound of Formula I or as defined in embodiment (1). In another embodiment, the Compound of Formula I is according to Formula I(el) or I(e2) where the R²¹ at the 1-position is methyl, ethyl, 2-hydroxyethyl, or 2-(N-methylamino)-ethyl and the R²¹ at the 2-position, when present, is methyl; and all other groups are as defined in the Summary of the Invention for a Compound of Formula I or as defined in embodiment (1).

### Embodiments (D2)

In another embodiment of the Invention, the Compound of Formula I is according to Formula I(el) or I(e2) where the R²¹ at the 1-position is alkyl or hydroxyalkyl and the second R²¹ is not present; and all other groups are as defined in the Summary of the Invention for a Compound of Formula I or as defined in embodiment (1). In another embodiment, the Compound of Formula I is according to Formula I(e1) or I(e2) where the R²¹ at the 1-position is methyl, ethyl, or 2-hydroxyethyl and the second R²¹ is not present; and all other groups are as defined in the Summary of the Invention for a Compound of Formula I or as defined in embodiment (1).

### Embodiments (D3)

In another embodiment of the Invention, the Compound of Formula I is according to Formula I(e1) or I(e2) where the R²¹ at the 1-position is alkyl and the second R²¹ is not present; and all other groups are as defined in the Summary of the Invention for a Compound of Formula I or as defined in embodiment (1). In another embodiment, the Compound of Formula I is according to Formula I(e1) or I(e2) where the R²¹ at the 1-position is methyl or ethyl and the second R²¹ is not present; and all other groups are as defined in the Summary of the Invention for a Compound of Formula I or as defined in embodiment (1).

### Embodiments (E)

In another embodiment of the Invention, the Compound of Formula I or Formula III is that where R¹ is unsubstituted benzimidazolyl; and all other groups are as defined in the Summary of the Invention for a Compound of Formula I or as defined in embodiment (1).

### Embodiment (E1)

In another embodiment, the Compound of Formula I is according to Formula I(f)

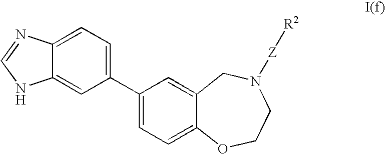

where Z is —C(O)—, and R² is as defined in the Summary of the Invention for a Compound of Formula I or as defined in embodiment (1).

### Embodiment (F)

In another embodiment, the Compound of Formula I or Formula III is that where R¹ is benzimidazolyl substituted with one, two, or three R²¹; and R²¹ and all other groups are as defined in the Summary of the Invention for a Compound of Formula I or as defined in embodiment (1).

### Embodiments (F1)

In another embodiment, the Compound of Formula I is according to Formula I(g)

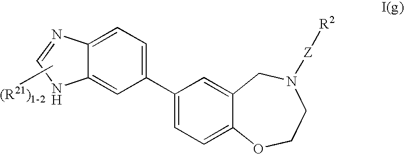

where each R²¹ is located at the 2-, 4-, or 5-positions of the benzimidazolyl ring, Z is —C(O)—, and R²¹ and R² are as defined in the Summary of the Invention for a Compound of Formula I. In another embodiment of the Invention, the Compound of Formula I is according to Formula I(g) where each R²¹ is located at the 2-, 4-, or 5-positions of the benzimidazolyl ring; one R²¹ is alkyl; halo; haloalkyl; alkoxyalkyl; hydroxyalkyl; optionally substituted cycloalkyl; optionally substituted heterocycloalkylalkyl; optionally substituted heteroarylalkyl; —C(O)OR²²; —NR²³R^{23a}; alkyl substituted with one —NR²³R^{23a}; —OR²⁴; —SR²⁵; —S(O)₂R²⁵; —NR^{23c}(O)OR^{24a}; —NR^{23c}(O)R^{23a}; alkyl substituted with one —NR²³C(O)R^{24a}; —C(O)NR²³R^{23a}; or —C(O)R^{24a}; the second R²¹, when present, is halo or alkyl; and R² and all other groups are as defined in the Summary of the Invention for a Compound of Formula I or as defined in embodiment (1).

### Embodiment (F2)

In another embodiment, the Compound of Formula I is according to Formula I(g) where one R²¹ is located at the 2-position of the R¹ benzimidazol-6-yl and is alkyl; halo; haloalkyl; alkoxyalkyl; hydroxyalkyl; optionally substituted cycloalkyl; optionally substituted heterocycloalkylalkyl; optionally substituted heteroarylalkyl; —C(O)OR²²; —NR²³R^{23a}; alkyl substituted with one —NR²³R^{23a}; —OR²⁴; —SR²⁵; —S(O)₂R²⁵; —NR²³C(O)OR^{24a}; —NR²³C(O)R^{23a}; alkyl substituted with one —NR²³C(O)R^{24a}; —C(O)NR²³R^{23a}; or —C(O)R^{24a}; the second R²¹, when present, is halo or alkyl and is located at the 4- or 5-position of the R¹ benzimidazol-6-yl; R²² is hydrogen; R²³ is hydrogen or alkyl; R^{23a} is hydrogen, alkyl, alkoxyalkyl, optionally substituted cycloalkyl, optionally substituted cycloalkylalkyl, aminoalkyl, alkylaminoalkyl, dialkylaminoalkyl, or optionally substituted phenyl; R^{24a} is alkyl; R²⁴ is hydrogen or alkyl; R²⁵ is alkyl; and all other groups are as defined in the Summary of the Invention for a Compound of Formula I or as defined in embodiment (1).

### Embodiment (F3)

In another embodiment, the Compound of Formula I is according to Formula I(g) where one R²¹ is located at the 2-position of the R¹ benzimidazol-6-yl and is methyl, ethyl, isopropyl, chloro, monofluoromethyl, difluoromethyl, trifluoromethyl, methoxymethyl, 2-methoxyethyl, hydroxymethyl, cyclopropyl, pyrrolidin-1-ylmethyl, pyrrolidin-2-ylmethyl, pyrrolidin-3-ylmethyl, imidazol-1-ylmethyl, imidazol-2-ylmethyl, imidazol-4-ylmethyl, imidazol-5-ylmethyl, carboxy, amino, methylamino, N,N-dimethylamino, 2-(N,N-dimethylamino)-ethylamino, N-methylaminomethyl, N-ethylaminomethyl, N,N-dimethylaminomethyl, 1-(N-methylamino)-ethyl, N-isopropylaminomethyl, N-cyclopropylaminomethyl, N-cyclopentylaminomethyl, N-(cyclopropylmethyl)-aminomethyl, N-(2-methoxy-ethyl)-aminomethyl, hydroxy, methoxy, ethoxy, methylthio, methylsulfonyl, methoxycarbonylamino, methylcarbonylamino, —NHC(O)CH₂N(CH₃)₂, —CH₂NHC(O)CH₃, aminocarbonyl, N-methylaminocarbonyl, N-ethylaminocarbonyl, N-n-propylaminocarbonyl, N-tert-butylaminocarbonyl, N-isopropylaminocarbonyl, N,N-dimethylaminocarbonyl, cyclopropylaminocarbonyl, cyclobutylaminocarbonyl, cyclohexylaminocarbonyl, phenylaminocarbonyl, or methylcarbonyl; the second R²¹, when present, is located at the 4- or 5-position and is fluoro or methyl; and all other groups are as defined in the Summary of the Invention for a Compound of Formula I or as defined in embodiment (1).

### Embodiments (F4)

In another embodiment, the Compound of Formula I is according to Formula I(g) where one R²¹ is located at the 2-position of the R¹ benzimidazol-6-yl and is alkyl; haloalkyl; hydroxyalkyl; —NR²³R^{23a}; alkyl substituted with one —NR²³R^{23a}; —OR²⁴; —C(O)NR²³R^{23a}; or —C(O)R^{24a}; the second R²¹, when present, is alkyl and is located at the 4-position of the R¹ benzimidazol-6-yl; R²³ is hydrogen; R^{23a}, R²⁴, and R^{24a} are alkyl; and all other groups are as defined in the Summary of the Invention for a Compound of Formula I or as defined in embodiment (1). In another embodiment of the Invention, the Compound of Formula I is according to Formula I(g) where one R²¹ is located at the 2-position of the R¹ benzimidazol-6-yl and is methyl, ethyl, monofluoromethyl, hydroxymethyl, amino, N-methylaminomethyl, ethoxy, N-methylaminocarbonyl, or methylcarbonyl; the second R²¹, when present, is located at the 4-position and is methyl; and all other groups are as defined in the Summary of the Invention for a Compound of Formula I or as defined in embodiment (1).

### Embodiment (F5)

In another embodiment, the Compound of Formula I is according to Formula I(g) where the R¹ benzimidazol-6-yl is substituted with one R²¹ at the 2-position of the R¹ benzimidazol-6-yl; R²¹ is methyl, ethyl, isopropyl, chloro, monofluoromethyl, difluoromethyl, trifluoromethyl, methoxymethyl, 2-methoxyethyl, hydroxymethyl, cyclopropyl, pyrrolidin-1-ylmethyl, pyrrolidin-2-ylmethyl, pyrrolidin-3-ylmethyl, imidazol-1-ylmethyl, imidazol-2-ylmethyl, imidazol-4-ylmethyl, imidazol-5-ylmethyl, carboxy, amino, methylamino, N,N-dimethylamino, 2-(N,N-dimethylamino)-ethylamino, N-methylaminomethyl, N-ethylaminomethyl, N,N-dimethylaminomethyl, 1-(N-methylamino)-ethyl, N-isopropylaminomethyl, N-cyclopropylaminomethyl, N-cyclopentylaminomethyl, N-(cyclopropylmethyl)-aminomethyl, N-(2-methoxy-ethyl)-aminomethyl, hydroxy, methoxy, ethoxy, methylthio, methylsulfonyl, methoxycarbonylamino, methylcarbonylamino, —NHC(O)CH₂N(CH₃)₂, —CH₂NHC(O)CH₃, aminocarbonyl, N-methylaminocarbonyl, N-ethylaminocarbonyl, N-n-propylaminocarbonyl, N-tert-butylaminocarbonyl, N,N-dimethylaminocarbonyl, 2-(N,N-dimethylamino)-ethylaminocarbonyl, cyclopropylaminocarbonyl, cyclobutylaminocarbonyl, phenylaminocarbonyl, or methylcarbonyl; and all other groups are as defined in the Summary of the Invention for a Compound of Formula I or as defined in embodiment (1).

### Embodiment (F6)

In another embodiment, the Compound of Formula I is according to Formula I(g) where the R¹ benzimidazol-6-yl is substituted with one R²¹ at the 2-position of the R¹ benzimidazol-6-yl; R²¹ is halo, alkyl, haloalkyl, hydroxyalkyl, —C(O)OR²², —SR²⁵, —NR²³C(O)OR^{24a}, —OR²⁴, —NR²³R^{23a}, —C(O)R^{24a}, —C(O)NR²³R^{23a}, cycloalkyl, or alkyl substituted with one —NR²³R^{23a}; R²² is hydrogen or alkyl; R²⁴, R^{24a}, and R²⁵ is alkyl; R²³ is hydrogen or alkyl; and R^{23a} is hydrogen, alkyl, or cycloalkyl; and all other groups are as defined in the Summary of the Invention for a Compound of Formula I or as defined in embodiment (1).

### Embodiment (F7)

In another embodiment, the Compound of Formula I is according to Formula I(j)

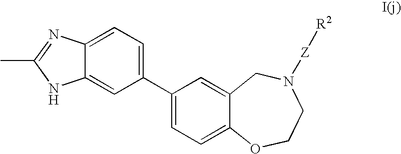

where Z is —C(O)—, and R² is as defined in the Summary of the Invention for a Compound of Formula I or as defined in embodiment (1).

### Embodiment (G)

In another embodiment, the Compound of Formula I or Formula III is that where R¹ is phenyl optionally substituted with one, two, or three R²⁰ where each R²⁰, independently of each other, and all other groups are as defined in the Summary of the Invention for a Compound of Formula I or as defined in embodiment (1). In another embodiment, the Compound of Formula I or Formula III is that where R¹ is phenyl optionally substituted with one, two, or three R²⁰ where each R²⁰ is independently nitro; cyano; halo; alkyl; haloalkyl; —NR¹⁵NR^{15a}; —NR¹⁵C(O)R¹⁸; —NR¹⁵S(O)₂R¹⁸; —OR⁹; heteroaryl optionally substituted with 1, 2, or 3 R²⁷; —C(O)OR⁹; —C(O)NR¹⁶R^{16a}; —NR¹⁵C(O)NR^{15b}R^{15a}; S(O)₂R¹⁷; alkyl substituted with —C(O)NR¹⁶R^{16a}; —C(O)R²⁶; or heterocycloalkyl optionally substituted with alkyl, alkoxycarbonyl, or phenylalkyl; and all other groups are as defined in the Summary of the Invention for a Compound of Formula I or as defined in embodiment (1).

### Embodiment (G1)

In another embodiment, the Compound of Formula I is according to Formula I(k)

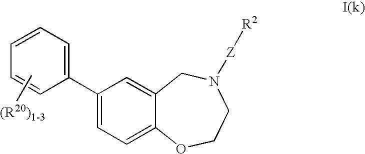

where Z is —C(O)— and R²⁰ and R² are as defined in the Summary of the Invention for a Compound of Formula I or as defined in embodiment (1).

### Embodiment (G2)

In another embodiment, the Compound of Formula I is according to Formula I(k) where one R²⁰ is nitro; halo; alkyl; haloalkyl; —NR¹⁵NR^{15a}; —NR¹⁵C(O)R¹⁸; —NR¹⁵S(O)₂R¹⁸; —OR⁹; heteroaryl optionally substituted with one or two R²⁷; —C(O)OR⁹; —C(O)R²⁶; —C(O)NR¹⁶R^{16a}; —NR¹⁵C(O)NR^{15b}R^{15a}; S(O)₂R¹⁷; alkyl substituted with —C(O)NR¹⁶R^{16a}; or heterocycloalkyl optionally substituted with alkyl, alkoxycarbonyl, or phenylalkyl; the second R²⁰, when present, is halo, alkyl, —NR¹⁵R^{15a}, or OR⁹; and the third R²⁰, when present, is halo; and all other groups are as defined in the Summary of the Invention for a Compound of Formula I or as defined in embodiment (1).

### Embodiment (G3)

In another embodiment, the Compound of Formula I is according to Formula I(k) where
one R²⁰ is nitro; halo; alkyl; haloalkyl; —NR¹⁵R^{15a}; —NR¹⁵C(O)R¹⁸; NR¹⁵S(O)₂R¹⁸; —OR⁹; heteroaryl optionally substituted with one or two R²⁷; —C(O)OR⁹; —C(O)R²⁶; —C(O)NR¹⁶R^{16a}; —NR¹⁵C(O)NR^{15b}R^{15a}; S(O)₂R¹⁷; alkyl substituted with —C(O)NR¹⁶R^{16a}; or heterocycloalkyl optionally substituted with alkyl, alkoxycarbonyl, or phenylalkyl;
the second R²⁰, when present, is halo, alkyl, —NR¹⁵R^{15a}, or OR⁹;
the third R²⁰, when present, is halo;
R⁹ is hydrogen, alkyl, haloalkyl, or alkyl substituted with one —NR¹⁰R^{10a};
R¹⁰; R^{10a}; R¹⁵, and R¹⁶ is hydrogen or alkyl;
R^{15a} is hydrogen, alkyl, haloalkyl, or dialkylaminoalkyl;
R^{15b} is alkyl;
R^{16a} is hydrogen; alkyl; haloalkyl; alkoxyalkyl; aminoalkyl; alkylaminoalkyl; dialkylaminoalkyl; heterocycloalkyl optionally substituted with alkyl; or optionally substituted heterocycloalkylalkyl;
R¹⁷ is amino, alkylamino, or dialkylamino;
R¹⁸ is alkyl, haloalkyl, or alkylaminoalkyl;
R²⁶ is optionally substituted heterocycloalkyl;
each R²⁷, when present, is independently alkyl, haloalkyl, amino, acylamino, halo, hydroxy, hydroxyalkyl, aminoalkyl, alkylaminoalkyl, dialkylaminoalkyl, or optionally substituted phenyl;
R² and all other groups are as defined in the Summary of the Invention for a Compound of Formula I or as defined in embodiment (1).

### Embodiment (G4)

In another embodiment, the Compound of Formula I is according to Formula I(k) where one R²⁰ is heteroaryl optionally substituted with one or two R²⁷; the second R²⁰, when present, is halo, alkyl, or haloalkyl; the third R²⁰ is not present; and R², R²⁷, and all other groups are as defined in the Summary of the Invention for a Compound of Formula I or as defined in embodiment (1). In another embodiment, the Compound of Formula I is according to Formula I(k) where one R²⁰ is thiazolyl, thiadiazolyl, isoxazolyl, oxaxzolyl, imidazolyl, 4H-1,2,4-triazolyl, 1H-1,2,3-triazolyl, pyridinyl, 3H-imidazo[4,5-b]pyridinyl, 9H-purinyl, 1H-imidazo[4,5-b]pyrazinyl, benzimidazolyl, pyrazolyl, or imidazo[2,1-b]thiazolyl, each of which is optionally substituted with one or two R²⁷; the second R²⁰, when present, is halo, alkyl, or haloalkyl; the third R²⁰ is not present; and R², R²⁷, and all other groups are as defined in the Summary of the Invention for a Compound of Formula I or as defined in embodiment (1).

### Embodiments (G5)

In another embodiment, the Compound of Formula I is according to Formula I(k) where one R²⁰ is thiazolyl optionally substituted with one R²⁷; the second and third R²⁰ are not present; and R², R²⁷, and all other groups are as defined in the Summary of the Invention for a Compound of Formula I or as defined in embodiment (1). In another embodiment, the Compound of Formula I is according to Formula I(k) where one R²⁰ is thiazolyl optionally substituted with one R²⁷ where R²⁷ is amino or acylamino; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted thiazol-2-yl, unsubstituted thiazol-4-yl, unsubstituted thiazol-5-yl, thiazol-2-yl substituted with one amino, thiazol-4-yl substituted with one amino, thiazol-5-yl substituted with one amino, thiazol-2-yl substituted with one —NHC(O)CH₃, thiazol-4-yl substituted with one —NHC(O)CH₃, or thiazol-5-yl substituted with one —NHC(O)CH₃; the second and third R²⁰ are not present; and R² and all other groups are as defined in the Summary of the Invention for a Compound of Formula I or as defined in embodiment (1).

### Embodiments (G6)

In another embodiment, the Compound of Formula I is according to Formula I(k) where one R²⁰ is imidazolyl optionally substituted with one or two R²⁷; the second and third R²⁰ are not present; and R²⁷, R², and all other groups are as defined in the Summary of the Invention for a Compound of Formula I or as defined in embodiment (1). In another embodiment, the Compound of Formula I is according to Formula I(k) where one R²⁰ is imidazolyl optionally substituted with one or two R²⁷; each R²⁷ is independently alkyl, hydroxyalkyl, hydroxy, halo, alkylaminoalkyl, phenyl, acylamino, or haloalkyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted imidazolyl, imidazolyl substituted with one or two alkyl, imidazolyl substituted with one hydroxyalkyl, imidazolyl substituted with one hydroxy, imidazolyl substituted with one halo, imidazolyl substituted with one alkylaminoalkyl, imidazolyl substituted with one phenyl, imidazolyl substituted with one acylamino, or imidazolyl substituted with one haloalkyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted imidazol-1-yl, unsubstituted imidazol-2-yl, unsubstituted imidazol-4-yl, unsubstituted imidazol-5-yl, imidazol-1-yl substituted with one methyl or ethyl, imidazol-2-yl substituted with one methyl or ethyl, imidazol-4-yl substituted with one methyl or ethyl, imidazol-5-yl substituted with one methyl or ethyl, imidazol-1-yl substituted with two methyl, imidazol-2-yl substituted with two methyl, imidazol-4-yl substituted with two methyl, imidazol-5-yl substituted with two methyl, imidazol-1-yl substituted with one hydroxy, imidazol-2-yl substituted with one hydroxy, imidazol-4-yl substituted with one hydroxy, imidazol-5-yl substituted with one hydroxy, imidazol-1-yl substituted with one hydroxymethyl, imidazol-2-yl substituted with one hydroxymethyl, imidazol-4-yl substituted with one hydroxymethyl, imidazol-5-yl substituted with one hydroxymethyl, imidazol-1-yl substituted with one chloro, imidazol-2-yl substituted with one chloro, imidazol-4-yl substituted with one chloro, imidazol-5-yl substituted with one chloro, imidazol-1-yl substituted with one —CH₂CH(CH₃), imidazol-2-yl substituted with one —CH₂CH(CH₃), imidazol-4-yl substituted with one —CH₂CH(CH₃), imidazol-5-yl substituted with one —CH₂CH(CH₃), imidazol-1-yl substituted with one phenyl, imidazol-2-yl substituted with one phenyl, imidazol-4-yl substituted with one phenyl, imidazol-5-yl substituted with one phenyl, imidazol-2-yl substituted with one acetylamino, imidazol-4-yl substituted with one acetylamino, imidazol-5-yl substituted with one acetylamino, imidazol-1-yl substituted with one trifluoromethyl, imidazol-2-yl substituted with one trifluoromethyl, imidazol-4-yl substituted with one trifluoromethyl, or imidazol-5-yl substituted with one trifluoromethyl; the second and third R²⁰ are not present; and R² and all other groups are as defined in the Summary of the Invention for a Compound of Formula I or as defined in embodiment (1).

### Embodiments (G7)

In another embodiment, the Compound of Formula I is according to Formula I(k) where one R²⁰ is 3H-imidazo[4,5-b]pyridinyl, 3H-imidazo[4,5-c]pyridinyl, 1H-imidazo[4,5-b]pyrazinyl, 1H-imidazo[4,5-b]pyrazinyl, or 9H-purinyl, each of which is optionally substituted with one R²⁷; the second and third R²⁰ are not present; and R²⁷ and R² and all other groups are as defined in the Summary of the Invention for a Compound of Formula I or as defined in embodiment (1). In another embodiment, the Compound of Formula I is according to Formula I(k) where one R²⁰ is unsubstituted 3H-imidazo[4,5-b]pyridinyl, unsubstituted 3H-imidazo[4,5-c]pyridinyl, unsubstituted 1H-imidazo[4,5-b]pyrazinyl, unsubstituted 1H-imidazo[4,5-b]pyrazinyl, unsubstituted 9H-purinyl, and 9H-purinyl substituted with one halo; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted 3H-imidazo[4,5-b]pyridin-2-yl, unsubstituted 3H-imidazo[4,5-b]pyridin-5-yl, unsubstituted 3H-imidazo[4,5-b]pyridin-6-yl, unsubstituted 3H-imidazo[4,5-b]pyridin-7-yl, unsubstituted 1H-imidazo[4,5-b]pyrazin-2-yl, unsubstituted 1H-imidazo[4,5-b]pyrazin-5-yl, unsubstituted 1H-imidazo[4,5-b]pyrazin-6-yl, 1H-imidazo[4,5-b]pyrazin-2-yl substituted with one bromo, 1H-imidazo[4,5-b]pyrazin-5-yl substituted with one bromo, 1H-imidazo[4,5-b]pyrazin-6-yl substituted with one bromo, unsubstituted 3H-imidazo[4,5-c]pyridin-1-yl, unsubstituted 3H-imidazo[4,5-c]pyridin-2-yl, unsubstituted 3H-imidazo[4,5-c]pyridin-4-yl, unsubstituted 3H-imidazo[4,5-c]pyridin-5-yl, unsubstituted 3H-imidazo[4,5-c]pyridin-7-yl, 9H-purin-2-yl, unsubstituted 9H-purin-6-yl, unsubstituted 9H-purin-8-yl, or unsubstituted 9H-purin-9-yl; the second and third R²⁰ are not present; and R² and all other groups are as defined in the Summary of the Invention for a Compound of Formula I or as defined in embodiment (1).

### Embodiments (G8)

In another embodiment, the Compound of Formula I is according to Formula I(k) where one R²⁰ is benzimidazolyl optionally substituted with one or two R²⁷; the second and third R²⁰ are not present; and R²⁷ and R² and all other groups are as defined in the Summary of the Invention for a Compound of Formula I or as defined in embodiment (1). In another embodiment, the Compound of Formula I is according to Formula I(k) where one R²⁰ is benzimidazolyl optionally substituted with one or two R²⁷ where each R²⁷ is independently alkyl, halo, or haloalkyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted benzimidazolyl, benzimidazolyl substituted with one or two halo, benzimidazolyl substituted with one or two alkyl, or benzimidazolyl substituted with one or two haloalkyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted benzimidazol-1-yl, unsubstituted benzimidazol-2-yl, unsubstituted benzimidazol-4-yl, unsubstituted benzimidazol-5-yl, unsubstituted benzimidazol-6-yl, unsubstituted benzimidazol-7-yl, benzimidazol-1-yl substituted with one or two fluoro, benzimidazol-2-yl substituted with one or two fluoro, benzimidazol-4-yl substituted with one or two fluoro, benzimidazol-5-yl substituted with one or two fluoro, benzimidazol-6-yl substituted with one or two fluoro, benzimidazol-7-yl substituted with one or two fluoro, benzimidazol-1-yl substituted with one or two chloro, benzimidazol-2-yl substituted with one or two chloro, benzimidazol-4-yl substituted with one or two chloro, benzimidazol-5-yl substituted with one or two chloro, benzimidazol-6-yl substituted with one or two chloro, benzimidazol-7-yl substituted with one or two chloro, benzimidazol-1-yl substituted with one or two bromo, benzimidazol-2-yl substituted with one or two bromo, benzimidazol-4-yl substituted with one or two bromo, benzimidazol-5-yl substituted with one or two bromo, benzimidazol-6-yl substituted with one or two bromo, benzimidazol-7-yl substituted with one or two bromo, benzimidazol-1-yl substituted with one chloro and one fluoro, benzimidazol-2-yl substituted with one chloro and one fluoro, benzimidazol-4-yl substituted with one chloro and one fluoro, benzimidazol-5-yl substituted with one chloro and one fluoro, benzimidazol-6-yl substituted with one chloro and one fluoro, benzimidazol-7-yl substituted with one chloro and one fluoro, benzimidazol-1-yl substituted with one or two methyl, benzimidazol-2-yl substituted with one or two methyl, benzimidazol-4-yl substituted with one or two methyl, benzimidazol-5-yl substituted with one or two methyl, benzimidazol-6-yl substituted with one or two methyl, benzimidazol-7-yl substituted with one or two methyl, benzimidazol-1-yl substituted with one trifluoromethyl, benzimidazol-2-yl substituted with one trifluoromethyl, benzimidazol-4-yl substituted with one trifluoromethyl, benzimidazol-5-yl substituted with one trifluoromethyl, benzimidazol-6-yl substituted with one trifluoromethyl, or benzimidazol-7-yl substituted with one trifluoromethyl; the second and third R²⁰ are not present; and R² and all other groups are as defined in the Summary of the Invention for a Compound of Formula I or as defined in embodiment (1).

### Embodiments (G9)

In another embodiment, the Compound of Formula I is according to Formula I(k) where one R²⁰ is pyrazolyl optionally substituted with one R²⁷; the second and third R²⁰ are not present; and R²⁷ and R² and all other groups are as defined in the Summary of the Invention for a Compound of Formula I or as defined in embodiment (1). In another embodiment, the Compound of Formula I is according to Formula I(k) where one R²⁰ is pyrazolyl optionally substituted with one R²⁷ where R²⁷ is alkyl, amino, or haloalkyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted pyrazolyl, pyrazolyl substituted with one alkyl, pyrazolyl substituted with one amino, or pyrazolyl substituted with one haloalkyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted pyrazol-1-yl, unsubstituted pyrazol-3-yl, unsubstituted pyrazol-4-yl, unsubstituted pyrazol-5-yl, pyrazol-1-yl substituted with one chloro, pyrazol-3-yl substituted with one chloro, pyrazol-4-yl substituted with one chloro, pyrazol-5-yl substituted with one chloro, pyrazol-1-yl substituted with one amino, pyrazol-3-yl substituted with one amino, pyrazol-4-yl substituted with one amino, pyrazol-5-yl substituted with one amino, pyrazol-1-yl substituted with one trifluoromethyl, pyrazol-3-yl substituted with one trifluoromethyl, pyrazol-4-yl substituted with one trifluoromethyl, or pyrazol-5-yl substituted with one trifluoromethyl; the second and third R²⁰ are not present; and R² and all other groups are as defined in the Summary of the Invention for a Compound of Formula I or as defined in embodiment (1).

### Embodiments (G10)

In another embodiment, the Compound of Formula I is according to Formula I(k) where one R²⁰ is triazolyl optionally substituted with one R²⁷; the second and third R²⁰ are not present; and R²⁷ and R² and all other groups are as defined in the Summary of the Invention for a Compound of Formula I or as defined in embodiment (1). In another embodiment, the Compound of Formula I is according to Formula I(k) where one R²⁰ is triazolyl optionally substituted with one R²⁷ where R²⁷ is hydroxy or alkyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted triazolyl, triazolyl substituted with one hydroxy, or triazolyl substituted with one alkyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted 1H-1,2,3-triazol-1-yl, unsubstituted 1H-1,2,3-triazol-4-yl, unsubstituted 1H-1,2,3-triazol-5-yl, unsubstituted 4H-1,2,4-triazol-3-yl, unsubstituted 4H-1,2,4-triazol-4-yl, unsubstituted 4H-1,2,4-triazol-5-yl, 5-oxo-1H-1,2,4-triazol-3-yl, 4-oxo-1,2,3-triazol-1-yl, 4-oxo-1,2,3-triazol-5-yl, 5-oxo-1,2,3-triazol-1-yl, 5-oxo-1,2,3-triazol-4-yl, 1H-1,2,3-triazol-1-yl substituted with methyl, 1H-1,2,3-triazol-4-yl substituted with methyl, 1H-1,2,3-triazol-5-yl substituted with methyl, 4H-1,2,4-triazol-3-yl substituted with methyl, 4H-1,2,4-triazol-4-yl substituted with methyl, or 4H-1,2,4-triazol-5-yl substituted with methyl; the second and third R²⁰ are not present; and R² and all other groups are as defined in the Summary of the Invention for a Compound of Formula I or as defined in embodiment (1).

### Embodiments (G11)

In another embodiment, the Compound of Formula I is according to Formula I(k) where one R²⁰ is unsubstituted imidazo[2,1-b]thiazolyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted imidazo[2,1-b]thiazol-2-yl, unsubstituted imidazo[2,1-b]thiazol-3-yl, unsubstituted imidazo[2,1-b]thiazol-5-yl, or unsubstituted imidazo[2,1-b]thiazol-6-yl; the second and third R²⁰ are not present; and R² and all other groups are as defined in the Summary of the Invention for a Compound of Formula I or as defined in embodiment (1).

### Embodiments (G12)

In another embodiment, the Compound of Formula I is according to Formula I(k) where one R²⁰ is thiadiazolyl optionally substituted with thiadiazolyl optionally substituted with one R²⁷ where R²⁷ is amino; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted 1,2,4-thiadiazol-3-yl, unsubstituted 1,2,4-thiadiazol-5-yl, unsubstituted 1,3,4-thiadiazol-2-yl, unsubstituted 1,3,4-thiadiazol-5-yl, unsubstituted 1,2,4-thiadiazol-3-yl substituted with one amino, 1,2,4-thiadiazol-5-yl substituted with one amino, 1,3,4-thiadiazol-2-yl substituted with one amino, or 1,3,4-thiadiazol-5-yl substituted with one amino; the second and third R²⁰ are not present; and R² and all other groups are as defined in the Summary of the Invention for a Compound of Formula I or as defined in embodiment (1).

### Embodiments (G13)

In another embodiment, the Compound of Formula I is according to Formula I(k) where one R²⁰ is oxazolyl or isoxazolyl optionally substituted with one R²⁷ where R²⁷ is amino; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted isoxazolyl, isoxazolyl substituted with one amino, unsubstituted oxazolyl, or oxazolyl substituted with one amino; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is unsubstituted isoxazol-3-yl, unsubstituted isoxazol-4-yl, unsubstituted isoxazol-5-yl, isoxazol-3-yl substituted with one amino, isoxazol-4-yl substituted with one amino, isoxazol-5-yl substituted with one amino, unsubstituted oxazol-2-yl, unsubstituted oxazol-4-yl, unsubstituted oxazol-5-yl, oxazol-2-yl substituted with one amino, oxazol-4-yl substituted with one amino, or oxazol-5-yl substituted with one amino; the second and third R²⁰ are not present; and R² and all other groups are as defined in the Summary of the Invention for a Compound of Formula I or as defined in embodiment (1).

### Embodiments (G14)

In another embodiment, the Compound of Formula I is according to Formula I(k) where one R²⁰ is pyridinyl optionally substituted with one R²⁷; the second and third R²⁰ are not present; and R²⁷ and R² and all other groups are as defined in the Summary of the Invention for a Compound of Formula I or as defined in embodiment (1). In another embodiment, the Compound of Formula I is according to Formula I(k) where one R²⁰ is unsubstituted pyridinyl or pyridinyl substituted with one amino; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is pyridin-2-yl, pyridin-3-yl, pyridin-4-yl, pyridin-2-yl substituted with one amino, pyridin-3-yl substituted with one amino, or pyridin-4-yl substituted with one amino; the second and third R²⁰ are not present; and R² and all other groups are as defined in the Summary of the Invention for a Compound of Formula I or as defined in embodiment (1).

### Embodiments (G15)

In another embodiment, the Compound of Formula I is according to Formula I(k) where one R²⁰ is 4,5-dihydro-1H-imidazolyl, 4,5-dihydro-1H-imidazolyl substituted with one alkyl, piperazinyl, piperazinyl substituted with alkyl, piperazinyl substituted with phenylalkyl, or morpholinyl; the second and third R²⁰ are not present; and R² and all other groups are as defined in the Summary of the Invention for a Compound of Formula I or as defined in embodiment (1). In another embodiment, the Compound of Formula I is according to Formula I(k) where one R²⁰ is 4,5-dihydro-1H-imidazol-1-yl, 4,5-dihydro-1H-imidazol-2-yl, 4,5-dihydro-1H-imidazol-4-yl, 4,5-dihydro-1H-imidazol-5-yl, 4,5-dihydro-1H-imidazol-1-yl substituted with one methyl, 4,5-dihydro-1H-imidazol-2-yl substituted with one methyl, 4,5-dihydro-1H-imidazol-4-yl substituted with one methyl, 4,5-dihydro-1H-imidazol-5-yl substituted with one methyl, piperazin-1-yl, piperazin-2-yl, piperazin-3-yl, piperazin-1-yl substituted with one methyl, piperazin-2-yl substituted with one methyl, piperazin-3-yl substituted with one methyl, piperazin-1-yl substituted with one phenylmethyl, piperazin-2-yl substituted with one phenylmethyl, piperazin-3-yl substituted with one phenylmethyl, morpholin-2-yl, morpholin-3-yl, or morpholin-4-yl; the second and third R²⁰ are not present; and R² and all other groups are as defined in the Summary of the Invention for a Compound of Formula I or as defined in embodiment (1).

### Embodiment (G16)

In another embodiment, the Compound of Formula I is according to Formula I(k) where one R²⁰ is nitro, alkyl, haloalkyl, —NR¹⁵R^{15a}, —OR⁹, —C(O)OR⁹, —C(O)NR¹⁶R^{16a}, —NR¹⁵C(O)R¹⁸, —NR¹⁵C(O)NR^{15a}R^{15b}, heterocycloalkyl optionally substituted with alkyl, alkyl substituted with —C(O)NR¹⁶R^{16a} or —NR¹⁵S(O)₂R¹⁸; the second R²⁰, when present, is —OR⁹, halo, alkyl, or —NR¹⁵R^{15a}; the third R²⁰, when present, is halo; and R² and all other groups are as defined in the Summary of the Invention for a Compound of Formula I or as defined in embodiment (1). In another embodiment, the Compound of Formula I is according to Formula I(k) where one R²⁰ is NO₂, NH₂, —NHCH₂CH₂N(CH₃)₂, —NHC(O)CH₂NHCH₃, —NHC(O)CH₃, —NHC(O)CF₃, —NHC(O)CH₂CH₂NHCH₃, —C(O)NH₂, —C(O)NH(CH₃), —C(O)NH(CH₂CH₃), —C(O)NH(CH₂CH₂CH₃), —C(O)NHCH₂CH(CH₃)₂, —C(O)NHCH₂CH₂F, —C(O)NHCH₂CHF₂, —C(O)NHCH₂CF₃, —C(O)NH(CH₂CH₂CF₃), —C(O)NHCH(CH₃)CF₃₅—C(O)NHCH₂CH₂N(CH₃)₂, —C(O)NHCH₂CH₂N(CH₂CH₃)₂, —C(O)NHCH₂CH₂OCH₃, azetidin-1-ylaminocarbonyl, azetidin-2-ylaminocarbonyl, azetidin-3-ylaminocarbonyl, N-methyl-azetidin-2-ylaminocarbonyl, N-methyl-azetidin-3-ylaminocarbonyl, azetidin-1-ylmethylaminocarbonyl, azetidin-2-ylmethylaminocarbonyl, azetidin-3-ylmethylaminocarbonyl, pyrrolidin-1-ylaminocarbonyl, pyrrolidin-2-ylaminocarbonyl, pyrrolidin-3-ylaminocarbonyl, pyrrolidin-1-ylmethylaminocarbonyl, pyrrolidin-2-ylmethylaminocarbonyl, pyrrolidin-3-ylmethylaminocarbonyl, piperidin-1-ylaminocarbonyl, piperidin-2-ylaminocarbonyl, piperidin-3-ylaminocarbonyl, piperidin-4-ylaminocarbonyl, piperidin-1-ylmethylaminocarbonyl, piperidin-2-ylmethylaminocarbonyl, piperidin-3-ylmethylaminocarbonyl, piperidin-4-ylmethylaminocarbonyl, morpholin-2-ylmethylaminocarbonyl, morpholin-3-ylmethylaminocarbonyl, morpholin-4-ylmethylaminocarbonyl, N-[(3aR,5r,6aS)-octahydrocyclopenta[c]pyrrol-5-ylmethyl]-aminocarbonyl, N—[N-(3aR,5r,6aS)-octahydrocyclopenta[c]pyrrol-5-ylmethyl]-aminocarbonyl, —CH₂C(O)NHCH₃, —NHC(O)NHCH₃, —NHC(O)N(CH₃)₂, —NHC(O)NHCH₂CH₂F, trifluoromethyl, —C(O)OCH₃, trifluoromethyl, trifluoromethoxy, —OCH₂CH₂N(CH₃)₂, N-(2-morpholinyl-ethyl)-aminocarbonyl, or —NHS(O)₂CH₃; the second R²⁰, when present, is methoxy, hydroxy, fluoro, chloro, methyl, ethyl, or NH₂; the third R²⁰, when present, is chloro; and R² and all other groups are as defined in the Summary of the Invention for a Compound of Formula I or as defined in embodiment (1).

### Embodiments (G17)

In another embodiment, the Compound of Formula I is according to Formula I(k) where one R²⁰ is amino, the second R²⁰ is amino, and the third R²⁰ is not present; and all other groups are as defined in the Summary of the Invention for a Compound of Formula I or as defined in embodiment (1). In another embodiment, the Compound of Formula I is according to Formula I(k) where one R²⁰ is —C(O)NR¹⁶R^{16a}, the second R²⁰ is not present or is halo, and the third R²⁰ is not present; and R¹⁶ and R^{16a} are as defined in the Summary of the Invention for a Compound of Formula I or as defined in embodiment (1). In another embodiment, the Compound of Formula I is according to Formula I(k) where one R²⁰ is amino, the second R²⁰ is haloalkyl, and the third R²⁰ is not present; and all other groups are as defined in the Summary of the Invention for a Compound of Formula I or as defined in embodiment (1). In another embodiment, the Compound of Formula I is according to Formula I(k) where one R²⁰ is amino, the second R²⁰ is alkyl, and the third R²⁰ is not present; and all other groups are as defined in the Summary of the Invention for a Compound of Formula I or as defined in embodiment (1). In another embodiment, the Compound of Formula I is according to Formula I(k) where one R²⁰ is —NR¹⁵C(O)NR^{15b}R^{15a}, and the second and third R²⁰ are not present; and all other groups are as defined in the Summary of the Invention for a Compound of Formula I or as defined in embodiment (1). In another embodiment, the Compound of Formula I is according to Formula I(k) where one R²⁰ is —NR¹⁵C(O)R¹⁸, the second R²⁰ is halo, and the third R²⁰ is not present; and all other groups are as defined in the Summary of the Invention for a Compound of Formula I or as defined in embodiment (1). In another embodiment, the Compound of Formula I is according to Formula I(k) where one R²⁰ is amino, the second R²⁰ is —OR⁹, and the third R²⁰ is not present; and all other groups are as defined in the Summary of the Invention for a Compound of Formula I or as defined in embodiment (1). In another embodiment, the Compound of Formula I is according to Formula I(k) where one R²⁰ is amino, the second R²⁰ is —OR⁹, and the third R²⁰ is not present; and R⁹ is hydrogen or alkyl; and all other groups are as defined in the Summary of the Invention for a Compound of Formula I or as defined in embodiment (1).

### Embodiment (H)

In another embodiment, the Compound of Formula I or Formula III is that where R¹ is a 6-membered heteroaryl optionally substituted with one or two R²¹; and R²¹ and all other groups are as defined in the Summary of the Invention for a Compound of Formula I or as defined in embodiment (1).

### Embodiment (H1)

In another embodiment, the Compound of Formula I or Formula III is that where R¹ is pyrimidinyl, pyrazinyl, or pyridazinyl, each of which is optionally substituted with one or two R²¹; and R²¹ and all other groups are as defined in the Summary of the Invention for a Compound of Formula I or as defined in embodiment (1).

### Embodiments (H2)

In another embodiment, the Compound of Formula I or Formula III is that where R¹ is pyrimidinyl optionally substituted with one R²¹; and R²¹ and all other groups are as defined in the Summary of the Invention for a Compound of Formula I or as defined in embodiment (1). In another embodiment, the Compound of Formula I or Formula III is that where R¹ is pyrimidin-5-yl optionally substituted with one R²¹; and R²¹ and all other groups are as defined in the Summary of the Invention for a Compound of Formula I or as defined in embodiment (1). In another embodiment of the Invention, the Compound of Formula I or Formula III is that where R¹ is pyrimidinyl optionally substituted with one R²¹ where R²¹ is —NR²³R^{23a}, —OR²⁴, —SR²⁵, or —S(O)₂R²⁵; and R²³, R^{23a}, R²⁴, R²⁵, and all other groups are as defined in the Summary of the Invention for a Compound of Formula I or as defined in embodiment (1). In another embodiment of the Invention, the Compound of Formula I or Formula III is that where R¹ is pyrimidin-5-yl optionally substituted with one R²¹ where R²¹ is —NR²³R^{23a}, —OR²⁴, or —SR²⁵; and R²³, R^{23a}, R²⁴, R²⁵, and all other groups are as defined in the Summary of the Invention for a Compound of Formula I or as defined in embodiment (1). In another embodiment of the Invention, the Compound of Formula I or Formula III is that where R¹ is pyrimidinyl optionally substituted with one R²¹ where R²¹ is —NR²³R^{23a}, —OR²⁴, or —SR²⁵; R²³ is hydrogen or alkyl; R^{23a} is hydrogen, alkyl, or dialkylaminoalkyl; R²⁴ and R²⁵ are alkyl; and all other groups are as defined in the Summary of the Invention for a Compound of Formula I or as defined in embodiment (1). In another embodiment of the Invention, the Compound of Formula I or Formula III is that where R¹ is pyrimidin-5-yl optionally substituted with one R²¹ where R²¹ is —NR²³R^{23a}, —OR²⁴, or —SR²⁵; R²³ is hydrogen or alkyl; R^{23a} is hydrogen, alkyl, or dialkylaminoalkyl; R²⁴ and R²⁵ are alkyl; and all other groups are as defined in the Summary of the Invention for a Compound of Formula I or as defined in embodiment (1).

### Embodiments (H3)

In another embodiment, the Compound of Formula I or Formula III is that where R¹ is pyridazinyl optionally substituted with one R²¹; and R²¹ and all other groups are as defined in the Summary of the Invention for a Compound of Formula I or as defined in embodiment (1). In another embodiment, the Compound of Formula I or Formula III is that where R¹ is pyridazin-3-yl optionally substituted with one R²¹; and R²¹ and all other groups are as defined in the Summary of the Invention for a Compound of Formula I or as defined in embodiment (1). In another embodiment, the Compound of Formula I or Formula III is that where R¹ is pyridazinyl optionally substituted with one R²¹ where R²¹ is —NR²³R^{23a}; and R²³, R^{23a}, and all other groups are as defined in the Summary of the Invention for a Compound of Formula I or as defined in embodiment (1). In another embodiment, the Compound of Formula I or Formula III is that where R¹ is pyridazin-3-yl optionally substituted with one R²¹ where R²¹ is —NR²³R^{23a}; and R²³, R^{23a}, and all other groups are as defined in the Summary of the Invention for a Compound of Formula I or as defined in embodiment (1). In another embodiment, the Compound of Formula I or Formula III is that where R¹ is pyridazinyl optionally substituted with one R²¹ where R²¹ is —NR²³R^{23a}; R²³ and R^{23a} are independently hydrogen or alkyl; and all other groups are as defined in the Summary of the Invention for a Compound of Formula I or as defined in embodiment (1). In another embodiment, the Compound of Formula I or Formula III is that where R¹ is pyridazin-3-yl optionally substituted with one R²¹ where R²¹ is —NR²³R^{23a}; R²³ and R^{23a} are independently hydrogen or alkyl; and all other groups are as defined in the Summary of the Invention for a Compound of Formula I or as defined in embodiment (1).

### Embodiments (H4)

In another embodiment, the Compound of Formula I or Formula III is that where R¹ is pyrazinyl optionally substituted with one R²¹; and R²¹ and all other groups are as defined in the Summary of the Invention for a Compound of Formula I or as defined in embodiment (1). In another embodiment, the Compound of Formula I or Formula III is that where R¹ is pyrazin-2-yl optionally substituted with one R²¹; and R²¹ and all other groups are as defined in the Summary of the Invention for a Compound of Formula I or as defined in embodiment (1). In another embodiment, the Compound of Formula I or Formula III is that where R¹ is pyrazinyl optionally substituted with one R²¹ where R²¹ is —NR²³R^{23a}; and R²³, R^{23a}, and all other groups are as defined in the Summary of the Invention for a Compound of Formula I or as defined in embodiment (1). In another embodiment, the Compound of Formula I or Formula III is that where R¹ is pyrazin-2-yl optionally substituted with one R²¹ where R²¹ is —NR²³R^{23a}; and R²³, R^{23a}, and all other groups are as defined in the Summary of the Invention for a Compound of Formula I or as defined in embodiment (1). In another embodiment, the Compound of Formula I or Formula III is that where R¹ is pyrazinyl optionally substituted with one R²¹ where R²¹ is —NR²³R^{23a}; R²³ and R^{23a} are independently hydrogen or alkyl; and all other groups are as defined in the Summary of the Invention for a Compound of Formula I or as defined in embodiment (1). In another embodiment, the Compound of Formula I or Formula III is that where R¹ is pyrazin-2-yl optionally substituted with one R²¹ where R²¹ is —NR²³R^{23a}; R²³ and R^{23a} are independently hydrogen or alkyl; and all other groups are as defined in the Summary of the Invention for a Compound of Formula I or as defined in embodiment (1).

### Embodiments (J)

In another embodiment, the Compound of Formula I or Formula III is that where R¹ is benzoxazolyl, benzoisoxazolyl, benzothiazolyl, or benzoisothiazolyl, each of which is optionally substituted with one, two, or three R²¹ groups; and R²¹ and all other groups are as defined in the Summary of the Invention for a Compound of Formula I or as defined in embodiment (1). In another embodiment of the Invention, the Compound of Formula I or Formula III is that where R¹ is benzoxazol-5-yl, benzoisoxazol-5-yl, benzothiazol-5-yl, benzoisothiazol-5-yl, benzoxazol-6-yl, benzoisoxazol-6-yl, benzothiazol-6-yl, or benzoisothiazol-6-yl, each of which is optionally substituted with one, two, or three R²¹ groups; and R²¹ and all other groups are as defined in the Summary of the Invention for a Compound of Formula I or as defined in embodiment (1).

### Embodiments (J1)

In another embodiment, the Compound of Formula I or Formula III is that where R¹ is benzothiazol-5-yl or benzothiazol-6-yl, each of which is optionally substituted with one R²¹ group where R²¹ is alkyl or NR²³R^{23a} where R²³ and R^{23a} are independently hydrogen or alkyl; and all other groups are as defined in the Summary of the Invention for a Compound of Formula I or as defined in embodiment (1). In another embodiment of the Invention, the Compound of Formula I or Formula III is that where R¹ is benzothiazol-5-yl or benzothiazol-6-yl each of which is optionally substituted with alkyl; and all other groups are as defined in the Summary of the Invention for a Compound of Formula I or as defined in embodiment (1). In another embodiment of the Invention, the Compound of Formula I or Formula III is that where R¹ is unsubstituted benzothiazol-5-yl or unsubstituted benzothiazol-6-yl; and all other groups are as defined in the Summary of the Invention for a Compound of Formula I or as defined in embodiment (1). In another embodiment of the Invention, the Compound of Formula I or Formula III is that where R¹ is benzothiazol-5-yl or benzothiazol-6-yl each of which is substituted with amino; and all other groups are as defined in the Summary of the Invention for a Compound of Formula I or as defined in embodiment (1).

### Embodiments (J2)

In another embodiment, the Compound of Formula I or Formula III is that where R¹ is benzoisoxazol-5-yl optionally substituted with one R²¹ group where R²¹ is alkyl or NR²³R^{23a} where R²³ and R^{23a} are independently hydrogen or alkyl; and all other groups are as defined in the Summary of the Invention for a Compound of Formula I or as defined in embodiment (1). In another embodiment of the Invention, the Compound of Formula I or Formula III is that where R¹ is unsubstituted benzoisoxazol-5-yl; and all other groups are as defined in the Summary of the Invention for a Compound of Formula I or as defined in embodiment (1).

### Embodiment (K1)

In another embodiment, the Compound of Formula I or Formula III is that where R¹ is indolyl, 1H-pyrrolo[2,3-b]pyridinyl, indazolyl, 1H-pyrazolo[3,4-b]pyridinyl, 1H-imidazo[4,5-b]pyridinyl, or imidazo[1,2-a]pyridinyl, each optionally substituted with one, two, or three R²¹; R²¹ and all other groups are as defined in the Summary of the Invention for a Compound of Formula I or as defined in embodiment (1).

### Embodiments (K2)

In another embodiment, the Compound of Formula I or Formula III is that where R¹ is indol-1-yl, indol-2-yl, indol-3-yl, indol-4-yl, indol-5-yl, indol-6-yl, or indol-7-yl, each optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is indol-3-yl or indol-5-yl, each optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is indol-3-yl or indol-5-yl, each optionally substituted with one R²¹; R²¹ is alkyl; and all other groups are as defined in the Summary of the Invention for a Compound of Formula I or as defined in embodiment (1).

### Embodiments (K3)

In another embodiment, the Compound of Formula I or Formula III is that where R¹ is 1H-pyrrolo[2,3-b]pyridin-1-yl, 1H-pyrrolo[2,3-b]pyridin-2-yl, 1H-pyrrolo[2,3-b]pyridin-3-yl, 1H-pyrrolo[2,3-b]pyridin-4-yl, 1H-pyrrolo[2,3-b]pyridin-5-yl, or 1H-pyrrolo[2,3-b]pyridin-6-yl, each of which is optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is 1H-pyrrolo[2,3-b]pyridin-5-yl optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is unsubstituted 1H-pyrrolo[2,3-b]pyridin-5-yl; and all other groups are as defined in the Summary of the Invention for a Compound of Formula I or as defined in embodiment (1).

### Embodiments (K4)

In another embodiment, the Compound of Formula I or Formula III is that where R¹ is 1H-indazol-1-yl, 1H-indazol-3-yl, 1H-indazol-4-yl, 1H-indazol-5-yl, 1H-indazol-6-yl, or 1H-indazol-7-yl, each of which is optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is 1H-indazol-3-yl, 1H-indazol-5-yl, or 1H-indazol-6-yl, each of which is optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is 1H-indazol-3-yl, 1H-indazol-5-yl, or 1H-indazol-6-yl, each of which is optionally substituted with one R²¹; R²¹ is alkyl; and all other groups are as defined in the Summary of the Invention for a Compound of Formula I or as defined in embodiment (1). In another embodiment of the Invention, the Compound of Formula I or Formula III is that where R¹ is unsubstituted 1H-indazol-3-yl, unsubstituted 1H-indazol-5-yl, or unsubstituted 1H-indazol-6-yl; and all other groups are as defined in the Summary of the Invention for a Compound of Formula I or as defined in embodiment (1).

### Embodiments (K5)

In another embodiment, the Compound of Formula I or Formula III is that where R¹ is 1H-pyrazolo[3,4-b]pyridin-1-yl, 1H-pyrazolo[3,4-b]pyridin-3-yl, 1H-pyrazolo[3,4-b]pyridin-4-yl, 1H-pyrazolo[3,4-b]pyridin-5-yl, or 1H-pyrazolo[3,4-b]pyridin-6-yl, each of which is optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is 1H-pyrazolo[3,4-b]pyridin-3-yl, 1H-pyrazolo[3,4-b]pyridin-5-yl, or 1H-pyrazolo[3,4-b]pyridin-6-yl, each of which is optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is unsubstituted 1H-pyrazolo[3,4-b]pyridin-3-yl, unsubstituted 1H-pyrazolo[3,4-b]pyridin-5-yl, or unsubstituted 1H-pyrazolo[3,4-b]pyridin-6-yl; and all other groups are as defined in the Summary of the Invention for a Compound of Formula I or as defined in embodiment (1).

### Embodiments (K6)

In another embodiment, the Compound of Formula I or Formula III is that where R¹ is imidazo[1,2-a]pyridin-2-yl, imidazo[1,2-a]pyridin-3-yl, imidazo[1,2-a]pyridin-5-yl, imidazo[1,2-a]pyridin-6-yl, imidazo[1,2-a]pyridin-7-yl, or imidazo[1,2-a]pyridin-8-yl, each of which is optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is imidazo[1,2-a]pyridin-6-yl, optionally substituted with one R²¹; R²¹ and all other groups are as defined in the Summary of the Invention for a Compound of Formula I or as defined in embodiment (1). In another embodiment of the Invention, the Compound of Formula I or Formula III is that where R¹ is imidazo[1,2-a]pyridin-6-yl, optionally substituted with one R²¹; R²¹ is NR²³R^{23a}; R²³ and R^{23a} are independently hydrogen or alkyl; and all other groups are as defined in the Summary of the Invention for a Compound of Formula I or as defined in embodiment (1). In another embodiment of the Invention, the Compound of Formula I or Formula III is that where R¹ is imidazo[1,2-a]pyridin-6-yl, optionally substituted with one R²¹; R²¹ is amino; and all other groups are as defined in the Summary of the Invention for a Compound of Formula I or as defined in embodiment (1).

### Embodiments (K7)

In another embodiment of the Invention, the Compound of Formula I or Formula III is that where R¹ is 1H-imidazo[4,5-b]pyridinyl optionally substituted with one R²¹; and R²¹ and all other groups are as defined in the Summary of the Invention for a Compound of Formula I or as defined in embodiment (1). In another embodiment of the Invention, the Compound of Formula I or Formula III is that where R¹ is 1H-imidazo[4,5-b]pyridin-6-yl optionally substituted with alkyl; and all other groups are as defined in the Summary of the Invention for a Compound of Formula I or as defined in embodiment (1). In another embodiment of the Invention, the Compound of Formula I or Formula III is that where R¹ is unsubstituted 1H-imidazo[4,5-b]pyridin-6-yl or is 2-methyl-1H-imidazo[4,5-b]pyridin-6-yl; and all other groups are as defined in the Summary of the Invention for a Compound of Formula I or as defined in embodiment (1).

### Embodiments (L)

In another embodiment, the Compound of Formula I or Formula III is that where R¹ is isoindolinyl optionally substituted with one R²¹; R²¹ and all other groups are as defined in the Summary of the Invention for a Compound of Formula I or as defined in embodiment (1). In another embodiment, the Compound of Formula I or Formula III is that where R¹ is isoindolin-5-yl optionally substituted with one R²¹; R²¹ and all other groups are as defined in the Summary of the Invention for a Compound of Formula I or as defined in embodiment (1). In another embodiment, the Compound of Formula I or Formula III is that where R¹ is isoindolin-5-yl optionally substituted with one R²¹; R²¹ is oxo; all other groups are as defined in the Summary of the Invention for a Compound of Formula I or as defined in embodiment (1).

In a Compound as described by Formula I or III, or in any of the above embodiments (1), A1-A7, B-B4, C-C7, D-D3, E, E1, F-F7, G-G17, H-H4, J-J2, K1-K7, and L, R² can be further described according to any of the following embodiments.

### Embodiments (N)

In another embodiment, R² is according to formula (m):

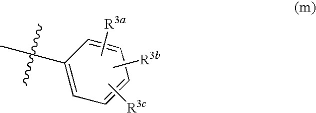

where R^{3a}, R^{3b}, and R^{3c} are as defined in the Summary of the Invention for a Compound of Formula I or as defined in embodiment (1). In another embodiment, R² is according to formula (m) where R^{3a} is halo, —S(O)₂R⁶ or —S(O)₂NR⁷R^{7a}; R^{3b} is halo, alkyl, or alkyl substituted with one —NR⁸R^{8a}; and R^{3c} is alkyl, halo, —OR⁹, or —NR¹¹R^{11a}; and R⁶, R⁷, R^{7a}, R⁸, R^{8a}, R⁹, R¹¹, and R^{11a} are as defined in the Summary of the Invention for a Compound of Formula I or as defined in embodiment (1).

### Embodiments (N1)

In another embodiment, R² is according to formula (n):

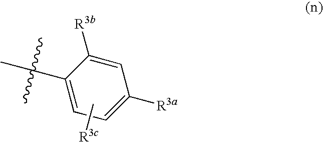

where R^{3a}, R^{3b}, and R^{3c} are as defined in the Summary of the Invention for a Compound of Formula I or as defined in embodiment (1). In another embodiment, R² is according to formula (n) where R^{3a} is halo, —S(O)₂R⁶, or —S(O)₂NR⁷R^{7a}; R^{3b} is halo, alkyl, or alkyl substituted with one —NR⁸R^{8a}; R^{3c} is alkyl, halo, —OR⁹, or —NR¹¹R^{11a}; and R⁶, R⁷, R^{7a}, R⁸, R^{8a}, R⁹, R¹¹, and R^{11a} and all other groups are as defined in the Summary of the Invention for a Compound of Formula I or as defined in embodiment (1). In another embodiment, R² is according to formula (n) where R^{3a} is halo, —S(O)₂R⁶, or —S(O)₂NR⁷R^{7a}; R^{3b} is halo, alkyl, or alkyl substituted with one —NR⁸R^{8a}; R^{3c} is alkyl, halo, —OR⁹, or —NR¹¹R^{11a}; and R⁶ is alkyl, alkenyl, haloalkyl, hydroxyalkyl, or phenyl optionally substituted with alkoxy; R⁷, R^{7a}, R⁸, and R^{8a} are independently hydrogen or alkyl; R⁹ is alkyl or haloalkyl; R¹¹ is hydrogen or alkyl; and R^{11a} is hydrogen or alkyl.

### Embodiments (N2)

In another embodiment, R² is according to formula (p):

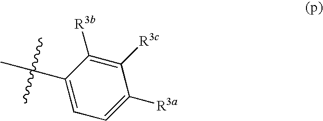

where R^{3a}, R^{3b}, and R^{3c} are as defined in the Summary of the Invention for a Compound of Formula I or as defined in embodiment (1). In another embodiment, R² is according to formula (p) where R^{3a} is —S(O)R⁶; R^{3b} is alkyl or alkyl substituted with one —NR⁸R^{8a}; R^{3c} is halo or —NR¹¹R^{11a}; and R⁶, R⁸, R^{8a}, R¹¹, and R^{11a} are as defined in the Summary of the Invention for a Compound of Formula I or as defined in embodiment (1). In another embodiment, R² is according to formula (p) is that where R^{3a} is —S(O)R⁶; R^{3b} is alkyl or alkyl substituted with one —NR⁸R^{8a}; R^{3c} is halo or —NR¹¹R^{11a}; where R⁶ is alkyl, alkenyl, hydroxyalkyl, haloalkyl, or phenyl optionally substituted with alkoxy; and R⁸, R^{8a}, R¹¹, and R^{11a} are independently hydrogen or alkyl.

### Embodiments (N3)

In another embodiment, R² is according to formula (p) where R^{3a} is —S(O)R⁶; R^{3b} is alkyl; R^{3c} is halo; and R⁶ is as defined in the Summary of the Invention for a Compound of Formula I or as defined in embodiment (1). In another embodiment, R² is according to formula (p) where R^{3a} is —S(O)R⁶; R^{3b} is methyl or ethyl; R^{3c} is halo; and R⁶ is methyl, ethyl, 2-hydroxy-ethyl, 3-hydroxy-propyl, di-fluoromethyl, mono-fluoromethyl, 2,2,2-trifluoroethyl, or 4-methoxyphenyl. In another embodiment, R² is according to formula (p) where R^{3a} is —S(O)R⁶; R^{3b} is alkyl; R^{3c} is halo; and R⁶ is alkyl. In another embodiment, R² is according to formula (p) where R^{3a} is —S(O)R⁶; R^{3b} is methyl or ethyl; R^{3c} is fluoro; and R⁶ is methyl or ethyl. In another embodiment, R² is according to formula (p) where R^{3a} is —S(O)R⁶; R^{3b} is methyl or ethyl; R^{3c} is fluoro; and R⁶ is methyl or ethyl.

### Embodiments (P)

In another embodiment, R² is according to formula (q)

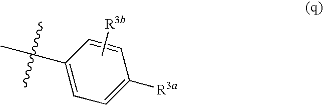

where R^{3a} and R^{3b} are as defined in the Summary of the Invention for a Compound of Formula I or as defined in embodiment (1). In another embodiment of the Invention, R² is according to formula (q) where R^{3a} is nitro, halo, alkyl, haloalkyl, —C(O)R²⁸, C(O)NR¹³R^{13a}; —S(O)₂R⁶, —S(O)₂NR⁷R^{7a}, —NR¹¹R^{11a}, or heteroaryl; R^{3b} is halo, alkyl, haloalkyl, —OR⁹, —NR¹¹R^{11a}, or —S(O)₂NR⁷R^{7a}; and R²⁸, R¹³, R^{13a}, R⁶, each R⁷ (independently), each R^{7a} (independently), each R¹¹ (independently), each R^{11a} (independently), and R⁹ are as defined in the Summary of the Invention for a Compound of Formula I or as defined in embodiment (1). In another embodiment, R² is according to formula (q) where R^{3a} is nitro, bromo, chloro, fluoro, iodo, methyl, trifluoromethyl, 2,2,2-trifluoroethyl, —C(O)R²⁸, C(O)NR¹³R^{13a}, —S(O)₂R⁶, —S(O)₂NR⁷R^{7a}, —NR¹¹R^{11a}, pyrrolyl, pyrrolyl substituted with one trifluoromethyl, thiadiazolyl, thiazolyl, imidazolyl, oxazolyl, triazolyl, or pyrazolyl; and R^{3b} is bromo, chloro, fluoro, iodo, methyl, ethyl, propyl, trifluoromethyl, —OR⁹, —NR¹¹R^{11a}, or —S(O)₂NR⁷R^{7a}; where R²⁸ is haloalkyl; R¹³ is hydrogen or alkyl; R^{13a} is hydrogen, alkyl, or cycloalkyl; R⁶ is alkyl, or phenyl substituted with one or two groups which groups are independently halo or alkoxy; each R⁷ is independently hydrogen or alkyl; each R^{7a} is independently hydrogen, alkyl, or cycloalkyl; each R¹¹ is independently hydrogen or alkyl; each R^{11a} is independently hydrogen, alkyl, or alkylsulfonyl; and R⁹ is alkyl.

### Embodiments (P1)

In another embodiment, R² is according to formula (q) where R^{3a} is halo and R^{3b} is halo; R^{3a} is —S(O)₂R⁶ and R^{3b} is alkyl; R^{3a} is —S(O)₂R⁶ and R^{3b} is halo; R^{3a} is —S(O)₂R⁶ and R^{3b} is haloalkyl; R^{3a} is —S(O)₂NR⁷R^{7a} and R^{3b} is halo; R^{3a} is —S(O)₂NR⁷R^{7a} and R^{3b} is alkyl; R^{3a} is OR⁹ and R^{3b} is alkyl; R^{3a} is alkyl and R^{3b} is alkyl; R^{3a} is alkyl and R^{3b} is halo; R^{3a} is halo and R^{3b} is alkyl; R^{3a} is heteroaryl and R^{3b} is alkyl; R^{3a} is haloalkyl and R^{3b} is halo; R^{3a} is haloalkyl and R^{3b} is alkyl; or R^{3a} is —NR¹¹R^{11a} and R^{3b} is alkyl; and R⁶, R⁷, R^{7a}, R⁹, R¹¹, and R^{11a} are as defined in the Summary of the Invention for a Compound of Formula I or as defined in embodiment (1).

### Embodiments (Q)

In another embodiment, R² is according to formula (r)

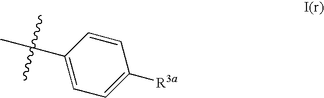

where R^{3a} is as defined in the Summary of the Invention for a Compound of Formula I or as defined in embodiment (1). In another embodiment of the Invention, R² is according to formula (r) where R^{3a} is nitro; cyano; halo; alkyl; alkynyl; cyanoalkyl; haloalkyl; haloalkyl substituted with 1, 2, or 3 hydroxy; alkylsulfonylalkyl; hydroxyalkyl; —C(O)R²⁸; —C(O)NR¹³R^{13a}; —C(O)C(O)NR²⁹R^{29a}; —SR¹⁴; —S(O)₂R⁶; —S(O)₂NR⁷R^{7a}; —OR⁹; —NR¹¹R^{11a}; alkyl substituted with one —NR⁸R^{8a}; phenyl; heteroaryl optionally substituted with one alkyl or haloalkyl; heteroarylalkyl; heterocycloalkyl optionally substituted with one alkyl, or cycloalkyl; and R²⁸, R¹³, R^{13a}, R²⁹, R^{29a}, R¹⁴, R⁶, R⁷, R^{7a}, R⁹, R¹¹, R^{11a}, R⁸, and R^{8a} are as defined in the Summary of the Invention for a Compound of Formula I or as defined in embodiment (1). In another embodiment, R² is according to formula (r) where R^{3a} is halo, alkyl, alkynyl, cyanoalkyl, haloalkyl, haloalkyl substituted with 1 or 2 hydroxy, hydroxyalkyl, —C(O)R²⁸, —SR¹⁴, —S(O)₂R⁶, —S(O)₂NR⁷R^{7a}, —OR⁹, —NR¹¹R^{11a}, —C(O)NR¹³R^{13a}, phenyl, heteroaryl, or cycloalkyl; and R²⁸, R¹⁴, R⁶, R⁷, R^{7a}, R⁹, R¹¹, R^{11a}, R¹³, and R^{13a} are as defined in the Summary of the Invention of or a Compound of Formula I or as defined in embodiment (1). In another embodiment, R² is according to formula (r) where R^{3a} is halo, alkyl, alkynyl, cyanoalkyl, haloalkyl, haloalkyl substituted with 1 or 2 hydroxy, hydroxyalkyl, —C(O)R²⁸, —SR¹⁴, —S(O)₂R⁶, —S(O)₂NR⁷R^{7a}, —OR⁹, —NR¹¹R^{11a}, —C(O)NR¹³R^{13a}, phenyl, heteroaryl, or cycloalkyl; where R⁶ is alkyl, haloalkyl, hydroxyalkyl, phenyl, phenyl substituted with one alkyl, phenyl substituted with one or two alkoxy, phenyl substituted with one halo and one alkoxy, heterocycloalkyl, heterocycloalkyl substituted with one alkyl, heterocycloalkylalkyl, cycloalkyl, or cycloalkylalkyl; R⁷ is hydrogen or alkyl; R^{7a} is hydrogen, alkyl, alkenyl, alkynyl, alkoxy, aminoalkyl, alkylsulfonylalkyl, hydroxyalkyl, heteroarylalkyl, or phenyl; R⁹ is haloalkyl; R¹¹ is hydrogen or alkyl; R^{11a} is hydrogen, alkyl, alkylsulfonyl, or phenylsulfonyl; R¹³ is hydrogen; R^{13a} is heterocycloalkylalkyl; R¹⁴ is alkyl; R²⁸ is alkyl, haloalkyl, or heterocycloalkyl.

### Embodiments (Q1)

In another embodiment, R² is according to formula (r) where
R^{3a} is nitro, cyano, bromo, chloro, fluoro, iodo, n-propyl, isopropyl, tert-butyl, n-butyl, isobutyl, n-pentyl, ethynyl, 1-cyano-1-methyl-ethyl, trifluoromethyl, 2,2,2-trifluoroethyl, 1-hydroxy-2,2,2-trifluoroethyl, 1,1,1,3,3,3-hexafluoro-2-hydroxy-prop-2-yl, 1,1-dihydroxy-2,2,2-trifluoroethyl, methylsulfonylmethyl, hydroxymethyl, 1-hydroxyethyl, 2-hydroxyethyl, —C(O)R²⁸, —C(O)NR¹³R^{13a}, —C(O)C(O)NR²⁹R^{29a}, —SR¹⁴; —S(O)₂R⁶, —S(O)₂NR⁷R^{7a}, —OR⁹; —NR¹¹R^{11a}, alkyl substituted with one —NR⁸R^{8a}, phenyl, pyrrolyl, pyrrolyl substituted with one trifluoromethyl, thiadiazolyl, thiazolyl, imidazolyl, oxazolyl, triazolyl, pyrazolyl, benzimidazol-2-ylmethyl, benzimidazol-1-ylmethyl, morpholinyl, piperazinyl, piperazinyl substituted with one methyl, or cyclohexyl;
R⁶ is alkyl, haloalkyl, hydroxyalkyl, alkyl substituted with one —NR¹⁰R^{10a}, alkyl substituted with one heterocycloalkyloxy, cycloalkyl, cycloalkylalkyl, heterocycloalkyl optionally substituted with one alkyl, heterocycloalkylalkyl, or phenyl optionally substituted with 1 or 2 groups which groups are independently halo, alkyl, amino, alkylamino, dialkylamino or alkoxy;
R⁷, R⁸, R¹⁰, R¹¹, R¹³, R²⁹, and R^{29a} are independently hydrogen or alkyl;
R^{7a} is hydrogen, alkyl, alkenyl, aminoalkyl, alkylaminoalkyl, dialkylaminoalkyl, hydroxyalkyl, alkylsulfonylalkyl, alkoxy, cycloalkyl, phenyl, heterocycloalkylalkyl, or heteroarylalkyl;
R^{8a} is independently hydrogen, alkyl, or alkoxycarbonyl;
R⁹ is alkyl, haloalkyl, hydroxyalkyl, phenyl, or alkyl substituted with one or two —NR¹⁰R^{10a};
R^{10a} is hydrogen, alkyl, hydroxyalkyl, or alkoxycarbonyl;
R^{11a} is alkyl, alkylsulfonyl, or phenylsulfonyl;
R^{13a} alkyl, haloalkyl, cycloalkyl, cycloalkylalkyl, heterocycloalkylalkyl, phenyl, or phenylalkyl;
R¹⁴ is alkyl, haloalkyl, or phenyl optionally substituted with one alkyl; and
R²⁸ is alkyl, haloalkyl, alkoxy, heterocycloalkyl optionally substituted with one alkyl, or phenyl.

### Embodiments (Q2)

In another embodiment, R² is according to formula (r) where R^{3a} is —S(O)₂R⁶; and R⁶ is as defined in the Summary of the Invention for a Compound of Formula I or as described in embodiment (1). In another embodiment, R² is according to formula (r) where R^{3a} is —S(O)₂R⁶; and R⁶ is alkyl. In another embodiment, R² is according to formula (r) where R^{3a} is —S(O)₂NR⁷R^{7a}; and R⁷ and R^{7a} are as defined in the Summary of the Invention for a Compound of Formula I or as described in embodiment (1). In another embodiment, R² is according to formula (r) where R^{3a} is halo. In another embodiment, R² is according to formula (r) where R^{3a} is haloalkyl optionally substituted with one or two hydroxy. In another embodiment, R² is according to formula (r) where R^{3a} is alkyl. In another embodiment, R² is according to formula (r) where R^{3a} is 5-membered heteroaryl optionally substituted with one haloalkyl. In another embodiment, R² is according to formula (r) where R^{3a} is oxazolyl, pyrazolyl, thiadiazolyl, imidazolyl, or thiazolyl, each of which is optionally substituted with one haloalkyl. In another embodiment, R² is according to formula (r) where R^{3a} is —SR¹⁴; and R¹⁴ is as defined in the Summary of the Invention for a Compound of Formula I or as described in embodiment (1). In another embodiment, R² is according to formula (r) where R^{3a} is —C(O)R²⁸; and R²⁸ is as defined in the Summary of the Invention for a Compound of Formula I or as described in embodiment (1). In another embodiment, R² is according to formula (r) where R^{3a} is —NR¹¹R^{11a}; and R¹¹ is hydrogen or alkyl and R^{11a} is phenylsulfonyl.

### Embodiments (R)

In another embodiment, R² is naphthyl substituted with R^{3a}, R^{3b}, R^{3c}, and R^{3d}; and R^{3a}, R^{3b}, R^{3c}, and R^{3d} are as defined in the Summary of the Invention for a Compound of Formula I or as described in embodiment (1). In another embodiment of the Invention, R² is naphthyl substituted with R^{3a}, R^{3b}, R^{3c}, and R^{3d}; R^{3a} is —S(O)₂R⁶ or —OR⁹; R^{3b}, R^{3c}, and R^{3d} are hydrogen; and R⁶ and R⁹ are as defined in the Summary of the Invention for a Compound of Formula I or as described in embodiment (1). In another embodiment of the Invention, R² is naphthyl substituted with R^{3a}, R^{3b}, R^{3c}, and R^{3d}; R^{3a} is —S(O)₂R⁶ or —OR⁹; R^{3b}, R^{3c}, and R^{3d} are hydrogen; R⁶ is alkyl; R⁹ is hydrogen or alkyl.

### Embodiments (S)

In another embodiment, R² is HET¹ optionally substituted with R^{4a}, R^{4b}, and R^{4c}; and HET¹, R^{4a}, R^{4b}, and R^{4c} are as defined in the Summary of the Invention for a Compound of Formula I or as described in embodiment (1). In another embodiment of the Invention, R² is HET¹ optionally substituted with R^{4a}, R^{4b}, and R^{4c}; R^{4a} is hydrogen; halo; alkyl; haloalkyl; —C(O)R¹²; —C(O)NR¹³R^{13a}; —S(O)₂R⁶; —S(O)₂NR⁷R^{7a}; —OR⁹; —NR¹¹R^{11a}; cycloalkyl; phenyl optionally substituted with 1 or 2 groups which groups are independently halo, alkyl, alkylsulfonyl, or alkoxy; heteroaryl; heteroarylalkyl; or heterocycloalkyl optionally substituted with 1, 2, or 3 groups which groups are independently alkyl or alkoxycarbonyl; R^{4b}, when R^{4b} is present, is hydrogen, alkyl, or haloalkyl; R^{4a}, when R^{4c} is present, is hydrogen or alkyl; and R¹², R¹³, R^{13a}, R⁶, R⁷, R^{7a}, R⁹, R¹¹, and R^{11a} are as defined in the Summary of the Invention for a Compound of Formula I or as described in embodiment (1). In another embodiment, HET¹ is pyrrolyl, thienyl, pyrazolyl, or thiazolyl, each of which is optionally substituted with R^{4a}, R^{4b}, and R^{4c}; R^{4a}, when R^{4a} is present, is alkyl, cycloalkyl, —C(O)R¹², or —S(O)₂R⁶; R^{4b}, when R^{4b} is present, is halo or alkyl; R^{4c}, when R^{4c} is present, is alkyl; and R¹² and R⁶ are as defined in the Summary of the Invention for a Compound of Formula I or as described in embodiment (1). In another embodiment, HET¹ is pyrrol-2-yl, pyrrol-3-yl, thien-2-yl, pyrazol-5-yl, thiazol-5-yl, each of which is optionally substituted with R^{4a}, R^{4b}, and R^{4c}; R^{4a}, when R^{4a} is present, is alkyl, cycloalkyl, —C(O)R¹², or —S(O)₂R⁶; R^{4b}, when R^{4b} is present, is halo or alkyl; R^{4c}, when R^{4c} is present, is alkyl; and R¹² and R⁶ are as defined in the Summary of the Invention for a Compound of Formula I or as described in embodiment (1).

### Embodiments (S1)

In another embodiment,
R² is thiazol-2-yl, thiazol-3-yl, thiazol-4-yl, isothiazol-2-yl, isothiazol-3-yl, isothiazol-4-yl, pyrrol-1-yl, pyrrol-2-yl, pyrrol-3-yl, pyrazol-1-yl, pyrazol-3-yl, pyrazol-4-yl, pyrazol-5-yl, thien-2-yl, thien-3-yl, 1,2,3-thiadiazol-4-yl, 1,2,3-thiadiazol-5-yl, 1,2,4-thiadiazol-3-yl, 1,2,4-thiadiazol-5-yl, 1,3,4-thiadiazol-2-yl, pyridin-2-yl, pyridin-3-yl, pyridin-4-yl, furan-2-yl, furan-3-yl, oxazol-2-yl, oxazol-4-yl, oxazol-5-yl, isoxazol-3-yl, isoxazol-4-yl, or isoxazol-5-yl; each of which is optionally substituted with R^{4a}, R^{4b}, and R^{4c};
R^{4a} is hydrogen, halo, methyl, ethyl, n-propyl, isopropyl, trifluoromethyl, —C(O)R¹², —C(O)NR¹³R^{13a}, —S(O)²R⁶, —S(O)₂NR⁷R^{7a}, —OR⁹, —NR¹¹R^{11a}, cyclopropyl, phenyl, phenyl substituted with 1 or 2 alkyl, phenyl substituted with 1 or 2 halo, phenyl substituted with 1 or 2 alkoxy, phenyl substituted with one alkylsulfonyl, phenyl substituted with one halo and one alkyl, phenyl substituted with one halo and one alkoxy, pyridinyl, pyrimidinyl, thienylmethyl, or heterocycloalkyl optionally substituted with one alkoxycarbonyl;
R^{4b}, when R^{4b} is present, is hydrogen, alkyl, or haloalkyl;
R^{4c}, when R^{4c} is present, is hydrogen or alkyl;
R⁷, R^{7a}, R¹¹, and R¹³ are independently hydrogen or alkyl;
R⁶ is alkyl, or heterocycloalkyl;
R¹² is hydroxy, alkyl, or alkoxy;
R⁹ is alkyl or haloalkyl;
R^{11a} is hydrogen, alkyl, alkoxycarbonyl, or alkylsulfonyl; and
R^{13a} is hydrogen, alkyl, or heterocycloalkylalkyl.

### Embodiments (S2)

In another embodiment, R² is HET² optionally substituted with R^{4a}, R^{4b}, R^{4c}, and R^{4d}; and HET², R^{4a}, R^{4b}, R^{4c}, R^{4d}, and all other groups are as defined in the Summary of the Invention for a Compound of Formula I. In another embodiment of the Invention, R² is according to formula (t) where R² is HET² optionally substituted with R^{4a}, R^{4b} and R^{4c}; R^{4a}, when R^{4a} is present, is halo, alkyl, cyanoalkyl, alkoxyalkyl, —C(O)R¹², —OR⁹, —S(O)₂R⁶, cyanoalkyl, or phenyl; R^{4b}, when R^{4b} is present, is halo or alkyl; R^{4c}, when R^{4c} is present, is halo; and R¹², R⁹, and R⁶ are as defined in the Summary of the Invention for a Compound of Formula I or as described in embodiment (1).

### Embodiments (S3)

In another embodiment,
R² is 4H-pyrrolo[3,2-c/]thiazol-2-yl, 4H-pyrrolo[3,2-d]thiazol-4-yl, 4H-pyrrolo[3,2-d]thiazol-5-yl, 4H-pyrrolo[3,2-d]thiazol-6-yl, 4H-pyrrolo[2,3-d]thiazol-2-yl, 4H-pyrrolo[2,3-d]thiazol-4-yl, 4H-pyrrolo[2,3-d]thiazol-5-yl, 4H-pyrrolo[2,3-d]thiazol-6-yl, indol-1-yl, indol-2-yl, indol-3-yl, indol-4-yl, indol-5-yl, indol-6-yl, indol-7-yl, 4H-furo[3,2-b]pyrrol-2-yl, 4H-furo[3,2-b]pyrrol-3-yl, 4H-furo[3,2-b]pyrrol-4-yl, 4H-furo[3,2-b]pyrrol-5-yl, 4H-furo[3,2-b]pyrrol-6-yl, 4H-thieno[3,2-b]pyrrol-2-yl, 4H-thieno[3,2-b]pyrrol-3-yl, 4H-thieno[3,2-b]pyrrol-4-yl, 4H-thieno[3,2-b]pyrrol-5-yl, 4H-thieno[3,2-b]pyrrol-6-yl, 6H-thieno[2,3-b]pyrrol-2-yl, 6H-thieno[2,3-b]pyrrol-3-yl, 6H-thieno[2,3-b]pyrrol-4-yl, 6H-thieno[2,3-b]pyrrol-5-yl, 6H-thieno[2,3-b]pyrrol-6-yl, benzo[b]thien-2-yl, benzo[b]thien-3-yl, benzo[b]thien-4-yl, benzo[b]thien-5-yl, benzo[b]thien-6-yl, benzo[b]thien-7-yl, 1H-pyrrolo[3,2-b]pyridin-1-yl, 1H-pyrrolo[3,2-b]pyridin-2-yl, 1H-pyrrolo[3,2-b]pyridin-3-yl, 1H-pyrrolo[3,2-b]pyridin-5-yl, 1H-pyrrolo[3,2-b]pyridin-6-yl, 1H-pyrrolo[3,2-b]pyridin-7-yl, 1H-pyrrolo[2,3-b]pyridin-2-yl, 1H-pyrrolo[2,3-b]pyridin-3-yl, 1H-pyrrolo[2,3-b]pyridin-4-yl, 1H-pyrrolo[2,3-b]pyridin-5-yl, 1H-pyrrolo[2,3-b]pyridin-6-yl, 1H-pyrrolo[2,3-c]pyridin-1-yl, 1H-pyrrolo[2,3-c]pyridin-2-yl, 1H-pyrrolo[2,3-c]pyridin-3-yl, 1H-pyrrolo[2,3-c]pyridin-4-yl, 1H-pyrrolo[2,3-c]pyridin-5-yl, 1H-pyrrolo[2,3-c]pyridin-7-yl, 1,1-dioxo-2,3-dihydrobenzo[b]thiophen-4-yl, 1,1-dioxo-2,3-dihydrobenzo[b]thiophen-5-yl, 1,1-dioxo-2,3-dihydrobenzo[b]thiophen-6-yl, 1,1-dioxo-2,3-dihydrobenzo[b]thiophen-7-yl, 1,1-dioxo-thiochroman-5-yl, 1,1-dioxo-thiochroman-6-yl, 1,1-dioxo-thiochroman-7-yl, 1,1-dioxo-thiochroman-8-yl, 1H-benzo[d][1,2,3]triazol-1-yl, 1H-benzo[d][1,2,3]triazol-4-yl, 1H-benzo[d][1,2,3]triazol-5-yl, 1H-benzo[d][1,2,3]triazol-6-yl, quinolin-2-yl, quinolin-3-yl, quinolin-4-yl, quinolin-5-yl, quinolin-6-yl, quinolin-7-yl, quinolin-8-yl, benzo[d][1,2,3]thiadiazol-4-yl, benzo[d][1,2,3]thiadiazol-5-yl, benzo[d][1,2,3]thiadiazol-6-yl, benzo[d][1,2,3]thiadiazol-7-yl, thieno[2,3-b]pyridin-2-yl, thieno[2,3-b]pyridin-3-yl, thieno[2,3-b]pyridin-4-yl, thieno[2,3-b]pyridin-5-yl, thieno[2,3-b]pyridin-6-yl, benzo[c]thiazol-2-yl, benzo[c]thiazol-4-yl, benzo[c]thiazol-5-yl, benzo[c]thiazol-6-yl, benzo[c]thiazol-7-yl, thieno[3,2-b]pyrazin-2-yl, thieno[3,2-b]pyrazin-3-yl, thieno[3,2-b]pyrazin-5-yl, thieno[3,2-b]pyrazin-6-yl, benzofuran-2-yl, benzofuran-3-yl, benzofuran-4-yl, benzofuran-5-yl, benzofuran-6-yl, benzofuran-7-yl, quinoxalin-2-yl, quinoxalin-3-yl, quinoxalin-4-yl, quinoxalin-5-yl, quinoxalin-6-yl, quinoxalin-7-yl, 1H-benzo[d]imidazol-1-yl, 1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, benzo[c]isoxazol-3-yl, benzo[c]isoxazol-4-yl, benzo[c]isoxazol-5-yl, benzo[c]isoxazol-6-yl, benzo[c]isoxazol-7-yl, benzo[d]isoxazole-3-yl, benzo[d]isoxazole-4-yl, benzo[d]isoxazole-5-yl, benzo[d]isoxazole-6-yl, benzo[d]isoxazole-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d][1,3]dioxol-4-yl, benzo[d][1,3]dioxol-5-yl, benzo[d][1,3]dioxol-6-yl, benzo[d][1,3]dioxol-7-yl, 2,3-dihydrobenzo[b][1,4]dioxin-5-yl, 2,3-dihydrobenzo[b][1,4]dioxin-6-yl, 2,3-dihydrobenzo[b][1,4]dioxin-7-yl, or 2,3-dihydrobenzo[b][1,4]dioxin-8-yl; each of which is optionally substituted with R^{4a}, R^{4b}, and R^{4c};
R^{4a}, when R^{4a} is present, is halo, alkyl, cyanoalkyl, alkoxyalkyl, —C(O)R¹², —OR⁹, —S(O)₂R⁶, or phenyl;
R^{4b}, when R^{4b} is present, is halo or alkyl;
R^{4c}, when R^{4c} is present, is halo;
R⁶, R⁹, and R¹² are alkyl.

### Embodiments (S4)

In another embodiment, R² is indol-2-yl, 1H-pyrrolo[2,3-b]pyridin-2-yl, 1H-pyrrolo[2,3-c]pyridin-2-yl, benzo[b]thien-2-yl, 4H-thieno[3,2-b]pyrrol-5-yl, 4H-furo[3,2-b]pyrrol-5-yl, 6H-thieno[2,3-b]pyrrol-5-yl, or 4H-pyrrolo[2,3-c]thiazol-5-yl, each of which is optionally substituted with R^{4a}, R^{4b}, and R^{4c}; R^{4a}, when R^{4a} is present, is halo, alkyl, alkoxyalkyl, —OR⁹, or —S(O)₂R⁶; R^{4b}, when R^{4b} is present, is alkyl or halo; R^{4c}, when R^{4c} is present is alkyl; and R⁹ and R⁶ are as defined in the Summary of the Invention for a Compound of Formula I or as described in embodiment (1).

In another embodiment, the Compound of Formula I(d) is according to any of embodiments (B)-(B4), and R² is as described in any of embodiments (N)-(N3), (P), (P1), (Q)-(Q2), (R), and (S)-S(4). In another embodiment, the Compound of Formula I(d) is according to any of embodiments (B)-(B4), and R² is as described in any of the embodiments (N)-(N3), (P1), (Q)-(Q2). In another embodiment, the Compound of Formula I(d) is according to any of embodiments (B4) and R² is as described in any of embodiments (N)-(N3), (P1), and (Q)-(Q2). In another embodiment, the Compound of Formula I(d) is according to any of the embodiments (B4) and R² is as described in any of embodiments (N2), (N3), (P1), (Q1), and (Q2). In another embodiment, the Compound of Formula I(d) is according to any of the embodiments (B4) and R² is as described in either of embodiments (N2) and (N3).

In another embodiment, the Compound of Formula I is according to any of the above embodiments (C)-(C7), and R² is as described in any of embodiments (N)-N3), (P), (P1), (Q)-(Q2), (R), (S)-S(4). In another embodiment, the Compound of Formula I is according to any of embodiments (C)-(C7), and R² is as described in any of the embodiments (N)-(N3), (P1), (Q), (Q1), and (Q2). In another embodiment, the Compound of Formula I is according to any of the embodiments (C3), (C5), and (C7), and R² is as described in any of embodiments (N)-(N3), (P1), and (Q)-(Q2). In another embodiment, the Compound of Formula I is according to any of the embodiments (C7) and R² is as described in any of embodiments (N2), (N3), (P1), (Q1), and (Q2). In another embodiment, the Compound of Formula I is according to any of the embodiments (C7) and R² is as described in any of embodiments (N2) and (N3).

In another embodiment, the Compound of Formula I(e1) or I(e2) is according to any of embodiments (D)-(D3) and R² is as described in any of embodiments (N)-(N3), (P), (P1), (Q)-(Q2), (R), and (S)-S(4). In another embodiment, the Compound of Formula I(e1) or I(e2) is according to any of the above embodiments (D)-(D3), and R² is as described in any of the embodiments (N2), (P1), (Q)-(Q2), and (S2)-(S4). In another embodiment, the Compound of Formula I(e1) or I(e2) is according to any of the embodiments (D3) and R² is as described in any of the embodiments (P1), (Q1), (Q2), and (S3). In another embodiment, the Compound of Formula I(e1) or I(e2) is according to any of the embodiments (B3) and R² is as described in any of the embodiments (N2), (N3), (P1), (Q1), and (Q2). In another embodiment, the Compound of Formula I(e1) or I(e2) is according to any of the embodiments (B3) and R² is as described in any of the embodiments (N2) and (N3).

In another embodiment, the Compound of Formula I is according to embodiment (E) or the Compound of Formula I(f) is according to embodiment (E1) and R² is as described in any of the embodiments (N)-(N3), (P), (P1), (Q)-(Q2), (R), and (S)-S(4). In another embodiment, the Compound of Formula I(f) is according to embodiment (E1) and R² is as described in any of the embodiments (N2), (N3), (P), (P1), (Q)-(Q2), (S)-S(3). In another embodiment, the Compound of Formula I(f) is according to embodiment (E1) and R² is as described in any of the embodiments (N2), (N3), (P), (P1), and (Q)-(Q2).

In another embodiment, the Compound of Formula I is according to any of the embodiments (F) or the Compound of Formula I(g) is according to any of the embodiments (F1)-(F7) and R² is as described in any of the embodiments (N)-(N3), (P), (P1), (Q)-(Q2), (R), (S)-S(4). In another embodiment, the Compound of Formula I is according to any of the embodiments (F) and (F1) and R² is as described in any of the embodiments (N2) and (N3). In another embodiment, the Compound of Formula I is according to any of the embodiments (F) and (F1) and R² is as described in any of the embodiments (P1). In another embodiment, the Compound of Formula I is according to any of the embodiments (F) and (F1) and R² is as described in any of the embodiments (Q1) and (Q2). In another embodiment, the Compound of Formula I is according to any of the embodiments (F) and (F1) and R² is as described in any of the embodiments (S1), (S3), and (S4). In another embodiment, the Compound of Formula I is according to any of the embodiments (F2) and (F3) and R² is as described in any of the embodiments (N2) and (N3). In another embodiment, the Compound of Formula I is according to any of the embodiments (F2) and (F3) and R² is as described in any of the embodiments (P1). In another embodiment, the Compound of Formula I is according to any of the embodiments (F2) and (F3) and R² is as described in any of the embodiments (Q1) and (Q2). In another embodiment, the Compound of Formula I is according to any of the embodiments (F2) and (F3) and R² is as described in any of the embodiments (S1), (S3), and (S4). In another embodiment, the Compound of Formula I is according to any of the embodiments (F2) and (F3) and R² is as described in any of the embodiments (P1). In another embodiment, the Compound of Formula I is according to any of the embodiments (F4) and R² is as described in any of the embodiments (Q1) and (Q2). In another embodiment, the Compound of Formula I is according to any of the embodiments (F5) and (F6) and R² is as described in any of the embodiments (N3), (P1), (Q1), (Q2), and (S4). In another embodiment, the Compound of Formula I(j) is that where R² is as described in any of the embodiments (N)-(N3), (P), (P1), (Q)-(Q2), (R), and (S)-S(4). In another embodiment, the Compound of Formula I(j) is that where R² is as described in any of the embodiments (N1)-(N3). In another embodiment, the Compound of Formula I(j) is that where R² is as described in any of the embodiments (P1). In another embodiment, the Compound of Formula I(j) is that where R² is as described in any of the embodiments (Q1) and (Q2). In another embodiment, the Compound of Formula I(j) is that where R² is as described in any of the embodiments (S1), (S3), and (S4).

In another embodiment, the Compound of Formula I is according to any of the embodiment (G) or the Compound of Formula I(k) is according to any of the embodiments (G2)-(G17) and R² is as described in any of the embodiments (N)-(N3), (P), (P1), (Q)-(Q2), (R), and (S)-S(4). In another embodiment, the Compound of Formula I(k) is according to any of the embodiments (G1)-(G17) and R² is as described in any of the embodiments (N3), (P1), (Q1), and (Q2). In another embodiment, the Compound of Formula I(k) is according to any of the embodiments (G4) and R² is as described in any of the embodiments (N3) and (Q2). In another embodiment, the Compound of Formula I(k) is according to any of the embodiments (G5), (G7), and (G10)-(G15) and R² is as described in any of the embodiments (N3). In another embodiment, the Compound of Formula I(k) is according to any of the embodiments (G6), (G16), and (G17) and R² is as described in any of the embodiments (N3), (Q1), and (Q2). In another embodiment, the Compound of Formula I(k) is according to any of the embodiments (G8) and R² is as described in any of the embodiments (N3), (P1), and (Q2). In another embodiment, the Compound of Formula I(k) is according to any of the embodiments (G9) and R² is as described in any of the embodiments (N3) and (P1).

In another embodiment, the Compound of Formula I is according to any of the embodiments (H) and R² is as described in any of the embodiments (N)-(N3), (P), (P1), (Q)-(Q2), (R), (S)-S(4). In another embodiment, the Compound of Formula I is according to any of the embodiments (H) and R² is as described in any of the embodiments (N)-(N3), (P), (P1), and (Q)-(Q2). In another embodiment, the Compound of Formula I is according to any of the embodiments (H) and R² is as described in any of the embodiments (N)-(N3).

In another embodiment, the Compound of Formula I is according to any of the embodiments (H1) and R² is as described in any of the embodiments (N)-(N3), (P), (P1), (O)-(Q2), (R), and (S)-S(4). In another embodiment, the Compound of Formula I is according to any of the embodiments (H1) and R² is as described in any of the embodiments (N)-(N3), (P), (P1), and (Q)-(Q2). In another embodiment, the Compound of Formula I is according to any of the embodiments (H1) and R² is as described in any of the embodiments (N)-(N3).

In another embodiment, the Compound of Formula I is according to any of the embodiments (H2) and R² is as described in any of the embodiments (N)-(N3), (P), (P1), (Q)-(Q2), (R), and (S)-S(4). In another embodiment, the Compound of Formula I is according to any of the embodiments (H2) and R² is as described in any of the embodiments (N)-(N3), (P), (P1), and (Q)-(Q2). In another embodiment, the Compound of Formula I is according to any of the embodiments (H2) and R² is as described in any of the embodiments (N)-(N3).

In another embodiment, the Compound of Formula I is according to any of the embodiments (H3) and R² is as described in any of the embodiments (N)-(N3), (P), (P1), (Q)-(Q2), (R), and (S)-S(4). In another embodiment, the Compound of Formula I is according to any of the embodiments (H3) and R² is as described in any of the embodiments (N)-(N3), (P), (P1), and (Q)-(Q2). In another embodiment, the Compound of Formula I is according to any of the embodiments (H3) and R² is as described in any of the embodiments (N)-(N3).

In another embodiment, the Compound of Formula I is according to any of the embodiments (H4) and R² is as described in any of the embodiments (N)-(N3), (P), (P1), (Q)-(Q2), (R), and (S)-S(4). In another embodiment, the Compound of Formula I is according to any of the embodiments (H4) and R² is as described in any of the embodiments (N)-(N3), (P), (P1), and (Q)-(Q2). In another embodiment, the Compound of Formula I is according to any of the embodiments (H4) and R² is as described in any of the embodiments (N)-(N3).

In another embodiment, the Compound of Formula I is according to any of the embodiments (J)-(J2) and R² is as described in any of the embodiments (N)-(N3), (P), (P1), (Q)-(Q2), (R), and (S)-S(4). In another embodiment, the Compound of Formula I is according to any of the embodiments (J) and R² is as described in any of the embodiments (N)-(N3), (P), (P1), and (Q)-(Q2). In another embodiment, the Compound of Formula I is according to any of the embodiments (J1) and R² is as described in any of the embodiments (N)-(N3), (P), (P1), and (Q)-(Q2). In another embodiment, the Compound of Formula I is according to any of the embodiments (J2) and R² is as described in any of the embodiments (N)-(N3), (P), (P1), and (Q)-(Q2). In another embodiment, the Compound of Formula I is according to any of the embodiments (J) and R² is as described in any of the embodiments (N2), (N3), (P1), and (Q2). In another embodiment, the Compound of Formula I is according to any of the embodiments (J1) and R² is as described in any of the embodiments (N2), (N3), (P1), and (Q2). In another embodiment, the Compound of Formula I is according to any of the embodiments (J2) and R² is as described in any of the embodiments (N2), (N3), (P1), and (Q2).

In another embodiment, the Compound of Formula I is according to any of the embodiments (K1)-(K7) and R² is as described in any of the embodiments (N)-N3), (P), (P1), (Q)-(Q2), (R), and (S)-S(4). In another embodiment, the Compound of Formula I is according to any of the embodiments (K1) and R² is as described in any of the embodiments (N2), (N3), (P1), and (Q2). In another embodiment, the Compound of Formula I is according to any of the embodiments and (K2) and R² is as described in any of the embodiments (N2), (N3), (P1), and (Q2). In another embodiment, the Compound of Formula I is according to any of the embodiments (K3) and R² is as described in any of the embodiments (N2), (N3), (P1), and (Q2). In another embodiment, the Compound of Formula I is according to any of the embodiments (K4) and R² is as described in any of the embodiments (N2), (N3), (P1), (Q1), and (Q2). In another embodiment, the Compound of Formula I is according to any of the embodiments (K5) and R² is as described in any of the embodiments (N2), (N3), (Q1), and (Q2). In another embodiment, the Compound of Formula I is according to any of the embodiments (K6) and R² is as described in any of the embodiments (N2) and (N3). In another embodiment of the Invention, the Compound of Formula I or Formula III is according to any of embodiments (K7) and R² is as described in any of the embodiments (N)-(N3), (P), (P1), (Q)-(Q2), (R), and (S)-S(4). In another embodiment of the Invention, the Compound of Formula I or Formula III is according to any of embodiments (K7) and R² is as described in any of the embodiments (N), (N2), (N3), and (Q)-(Q2). In another embodiment of the Invention, the Compound of Formula I or Formula III is according to any of embodiments (K7) and R² is as described in any of the embodiments (Q1) and (Q2).

In another embodiment, the Compound of Formula I is according to any of the embodiments (L) and R² is as described in any of the embodiments (N)-(N3), (P), (P1), (Q)-(Q2), (R), and (S)-S(4).

In another embodiment, the Compound of Formula I is according to Formula I(a)

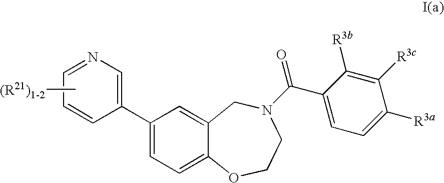

where one R²¹ is hydroxyalkyl; optionally substituted heteroaryl; —C(O)OR²²; —NR²³R^{23a}; —OR²⁴; —NR²³C(O)R^{23a}; —C(O)NR²³R^{23a}; —NR²³C(O)NR^{23a}R²⁴; —NR²³C(═NH)NR^{23a}R²⁴; or —NR²³S(O)₂R^{23a}; and
the second R²¹, when present, is halo, cyano, alkyl, or hydroxy;
R^{3a} is —S(O)R⁶; R^{3b} is alkyl or alkyl substituted with one —NR⁸R^{8a}; R^{3c} is halo or —NR¹¹R^{11a}; and all other groups are as defined in the Summary of the Invention for a Compound of Formula I.

In another embodiment, the Compound of Formula I is according to Formula I(b)

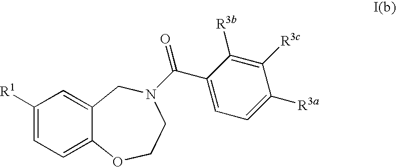

where
R¹ is a 5-membered heteroaryl or an N-oxide thereof, optionally substituted with one, two, or three R²¹ wherein each R²¹ is independently oxo, alkyl; halo; cyano; haloalkyl; alkoxy; alkoxyalkyl; hydroxyalkyl; optionally substituted cycloalkyl; optionally substituted cycloalkylalkyl; optionally substituted heterocycloalkyl; optionally substituted heterocycloalkylalkyl; optionally substituted heteroaryl; optionally substituted heteroarylalkyl; —C(O)OR²²; —NR²³R^{23a}; alkyl substituted with one —NR²³R^{23a}; —OR²⁴; —SR²⁵; —S(O)R²⁵; —S(O)₂R²⁵; —NR²³C(O)OR^{24a}; —NR²³C(O)R^{23a}; alkyl substituted with one —NR²³C(O)R^{24a}; alkyl substituted with phenylalkyloxy; —C(O)NR²³R^{23a}; —C(O)R^{24a}; —NR²³C(O)NR^{23a}R²⁴; —NR²³C(═NH)NR^{23a}R²⁴; or —NR²³S(O)₂R^{23a}; and
R^{3a} is —S(O)R⁶; R^{3b} is alkyl or alkyl substituted with one —NR⁸R^{8a}; R^{3c} is halo or —NR¹¹R^{11a}; and all other groups are as defined in the Summary of the Invention for a Compound of Formula I.

In another embodiment, the Compound of Formula I is according to Formula I(b), R¹ is oxazolyl, pyrazolyl, thienyl, thiazolyl, or thiadiazolyl, each of which is optionally substituted with one or two R²¹ wherein each R²¹ is independently alkyl, heteroaryl, —NR²³R^{23a}, —NR²³C(O)R^{23a}, or —C(O)NR²³R^{23a}; and where R^{3a} is —S(O)R⁶; R^{3b} is alkyl or alkyl substituted with one —NR⁸R^{8a}; R^{3c} is halo or —NR¹¹R^{11a}; and all other groups are as defined in the Summary of the Invention for a Compound of Formula I.

In another embodiment, the Compound of Formula I is according to Formula I(c1) or I(c2)

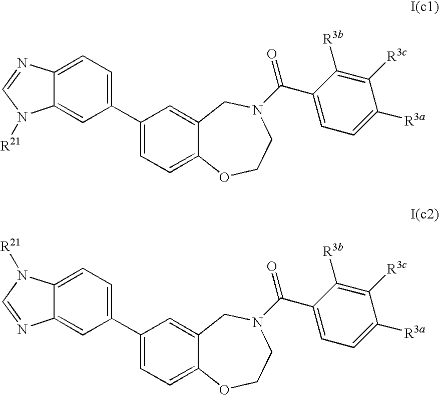

where R²¹ is alkyl, hydroxyalkyl, or alkyl substituted with one —NR²³R^{23a}; R^{3a} is —S(O)R⁶; R^{3b} is alkyl or alkyl substituted with one —NR⁸R^{8a}; R^{3c} is halo or —NR¹¹R^{11a}; and all other groups are as defined in the Summary of the Invention for a Compound of Formula I.

In another embodiment, the Compound of Formula I is according to Formula I(h)

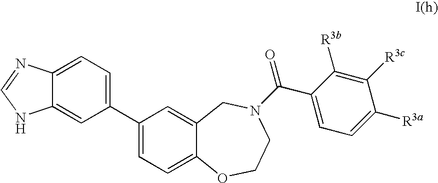

where R^{3a} is —S(O)R⁶; R^{3b} is alkyl or alkyl substituted with one —NR⁸R^{8a}; R^{3c} is halo or —NR¹¹R^{11a}; and all other groups are as defined in the Summary of the Invention for a Compound of Formula I.

In another embodiment, the Compound of Formula I is according to Formula I(m)

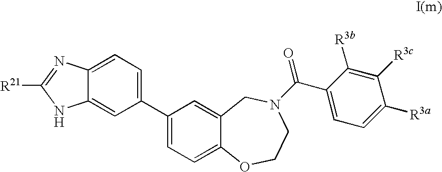

where R²¹ is halo, alkyl, haloalkyl, hydroxyalkyl, —C(O)OR²²; —SR²⁵, —NR²³C(O)OR^{24a}, —OR²⁴, —NR²³R^{23a}, —C(O)R^{24a}, —C(O)NR²³R^{23a}, cycloalkyl, or alkyl substituted with one —NR²³R^{23a}; R^{3a} is —S(O)R⁶; R^{3b} is alkyl or alkyl substituted with one —NR⁸R^{8a}; R^{3c} is halo or —NR¹¹R^{11a}; and all other groups are as defined in the Summary of the Invention for a Compound of Formula I.

In another embodiment, the Compound of Formula I is according to Formula I(n)

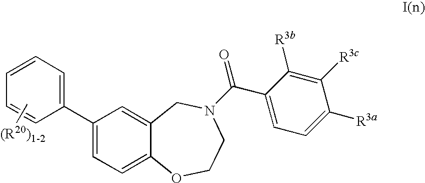

where R²⁰ is heteroaryl optionally substituted with one or two R²⁷; the second R²⁰, when present, is halo or alkyl; R^{3a} is —S(O)R⁶; R^{3b} is alkyl or alkyl substituted with one —NR⁸R^{8a}; R^{3c} is halo or —NR¹¹R^{11a}; and all other groups are as defined in the Summary of the Invention for a Compound of Formula I. In another embodiment, the Compound of Formula I is according to Formula I(n) where R²⁰ is thiazolyl optionally substituted with one or two R²⁷; the second R²⁰, when present, is halo or alkyl; R^{3a} is —S(O)R⁶; R^{3b} is alkyl or alkyl substituted with one —NR⁸R^{8a}; R^{3c} is halo or —NR¹¹R^{11a}; and all other groups are as defined in the Summary of the Invention for a Compound of Formula I.

Another embodiment comprises a pharmaceutical composition which comprises a compound of any one of Formula I and III or any one of the above embodiments or combinations of embodiments, optionally as a pharmaceutically acceptable salt thereof, and a pharmaceutically acceptable carrier, excipient, or diluent.

Another embodiment is a method of treating disease, disorder, or syndrome where the disease is associated with uncontrolled, abnormal, and/or unwanted cellular activities effected directly or indirectly by mTOR which method comprises administering to a human in need thereof a therapeutically effective amount of a Compound of Formula I or III, a Compound of any one of the above embodiments or combinations of embodiments, or a Compound in Table 1, optionally as a pharmaceutically acceptable salt or pharmaceutical composition thereof. In another embodiment the Compound is of Formula III.

Another embodiment is directed to a method of treating a disease, disorder, or syndrome which method comprises administering to a patient a therapeutically effective amount of a Compound of Formula I or III, a Compound of any one of the above embodiments or combinations of embodiments, or a Compound in Table 1, optionally as a pharmaceutically acceptable salt thereof, or a pharmaceutical composition comprising a therapeutically effective amount of a Compound of Formula I or III, a Compound of any one of the above embodiments or combinations of embodiments, or a Compound in Table 1, and a pharmaceutically acceptable carrier, excipient, or diluent.

### Embodiment T

Another embodiment of the invention is a method of treating cancer which method comprises administering to a patient a therapeutically effective amount of a Compound of Formula I or III, a Compound of any one of the above embodiments or combinations of embodiments, or a Compound in Table 1, optionally as a pharmaceutically acceptable salt thereof, or a pharmaceutical composition comprising a therapeutically effective amount of a Compound of Formula I or III, a Compound of any one of the above embodiments or combinations of embodiments, or a Compound in Table 1 and a pharmaceutically acceptable carrier, excipient, or diluent in combination with one or more chemotherapeutic agent(s).

In another embodiment of Embodiment T, the disease is cancer. In another embodiment, the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, non small cell lung carcinoma, small cell carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, or head and neck cancer. In another embodiment, the Compound is of Formula III.

In another embodiment of Embodiment T, the disease is hamaratoma, angiomyelolipomas, TSC-associated and sporadic lymphangioleiomyomatosis, multiple hamaratoma syndrome, neurofibromatosis, macular degeneration, macular edema, systemic lupus, or autoimmune lymphoproliferative syndrome. In another embodiment, the Compound is of Formula III.

Another aspect of the invention is a method of inhibiting proliferative activity in a cell, the method comprising administering to a cell or a plurality of cells an effective amount of a compound of Formula I, or a pharmaceutically acceptable salt, solvate, or prodrug thereof, or pharmaceutical composition thereof.

### Representative Compounds

Representative compounds of Formula I are depicted below. The examples are merely illustrative and do not limit the scope of the invention in any way. Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Specifically, names in Table 1 were generated using ACD/Labs naming software 8.00 release, product version 8.08 or higher.

TABLE 1

| Entry | Structure | Name |
|---|---|---|
| 1 | 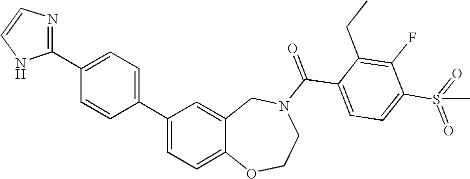 | 4-{[2-ethyl-3-fluoro-4- (methylsulfonyl)phenyl]carbonyl}- 7-[4-(1H-imidazol-2-yl)phenyl]- 2,3,4,5-tetrahydro- 1,4-benzoxazepine |
| 2 | 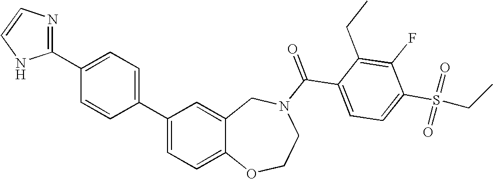 | 4-{[2-ethyl-4-(ethylsulfonyl)-3- fluorophenyl]carbonyl}-7-[4- (1H-imidazol-2-yl)phenyl]- 2,3,4,5-tetrahydro-1,4- benzoxazepine |
| 3 | 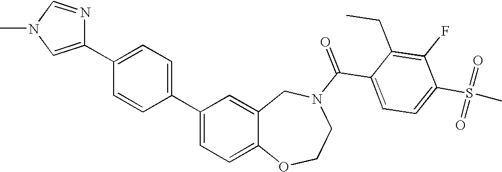 | 4-{[2-ethyl-3-fluoro-4- (methylsulfonyl)phenyl]carbonyl}- 7-[4-(1-methyl-1H-imidazol-4- yl)phenyl]-2,3,4,5-tetrahydro- 1,4-benzoxazepine |
| 4 | 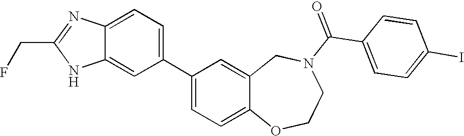 | 7-[2-(fluoromethyl)-1H- benzimidazol-6-yl]-4-[(4- iodophenyl)carbonyl]-2,3,4,5- tetrahydro-1,4-benzoxazepine |
| 5 | 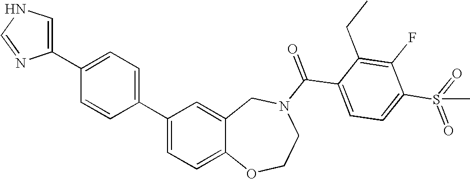 | 4-{[2-ethyl-3-fluoro-4- (methylsulfonyl)phenyl]carbonyl}- 7-[4-(1H-imidazol-4- yl)phenyl]-2,3,4,5-tetrahydro- 1,4-benzoxazepine |
| 6 | 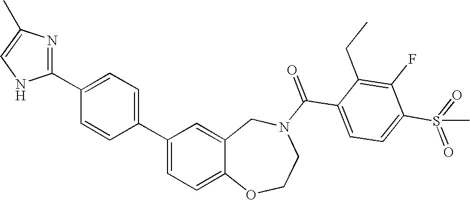 | 4-{[2-ethyl-3-fluoro-4- (methylsulfonyl)phenyl]carbonyl}- 7-[4-(4-methyl-1H-imidazol-2- yl)phenyl]-2,3,4,5-tetrahydro- 1,4-benzoxazepine |
| 7 | 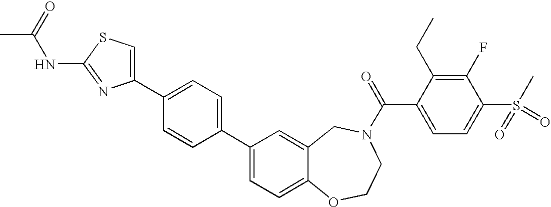 | N-{4-[4-(4-{[2-ethyl-3-fluoro-4- (methylsulfonyl)phenyl]carbonyl}- 2,3,4,5-tetrahydro-1,4- benzoxazepin-7-yl)phenyl]-1,3- thiazol-2-yl}acetamide |
| 8 | 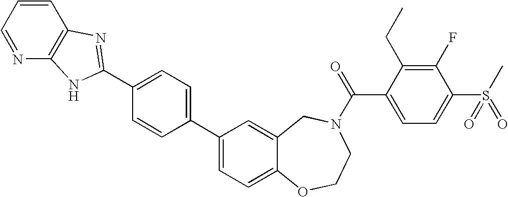 | 4-{[2-ethyl-3-fluoro-4- (methylsulfonyl)phenyl]carbonyl}- 7-[4-(3H-imidazo[4,5- b]pyridin-2-yl)phenyl]-2,3,4,5- tetrahydro-1,4-benzoxazepine |
| 9 | 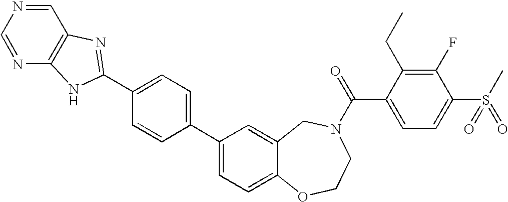 | 4-{[2-ethyl-3-fluoro-4- (methylsulfonyl)phenyl]carbonyl}- 7-[4-(9H-purin-8-yl)phenyl]- 2,3,4,5-tetrahydro-1,4- benzoxazepine |
| 10 | 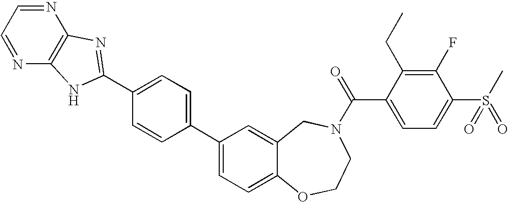 | 4-{[2-ethyl-3-fluoro-4- (methylsulfonyl)phenyl]carbonyl}- 7-[4-(1H-imidazo[4,5- b]pyrazin-2-yl)phenyl]-2,3,4,5- tetrahydro-1,4-benzoxazepine |
| 11 | 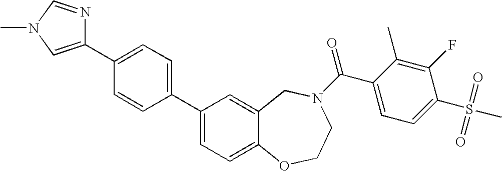 | 4-{[3-fluoro-2-methyl-4- (methylsulfonyl)phenyl]carbonyl}- 7-[4-(1-methyl-1H-imidazol-4- yl)phenyl]-2,3,4,5-tetrahydro- 1,4-benzoxazepine |
| 12 | 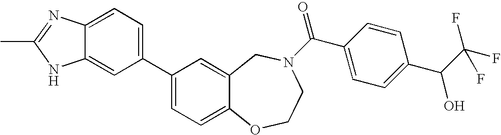 | 2,2,2-trifluoro-1-(4-{[7-(2- methyl-1H-benzimidazol-6-yl)- 2,3-dihydro-1,4-benzoxazepin- 4(5H- yl]carbonyl}phenyl)ethanol |
| 13 | 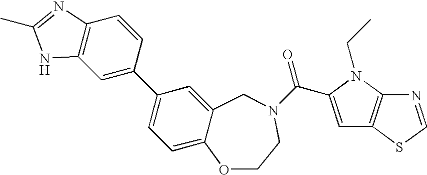 | 4-[(4-ethyl-4H-pyrrolo[2,3- d][1,3]thiazol-5-yl)carbonyl]-7- (2-methyl-1H-benzimidazol-6- yl)-2,3,4,5-tetrahydro-1,4- benzoxazepine |
| 14 | 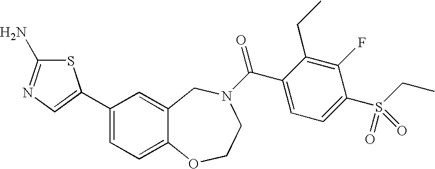 | 5-(4-{[2-ethyl-4-(ethylsulfonyl)- 3-fluorophenyl]carbonyl}- 2,3,4,5-tetrahydro-1,4- benzoxazepin-7-yl)-1,3-thiazol- 2-amine |
| 15 | 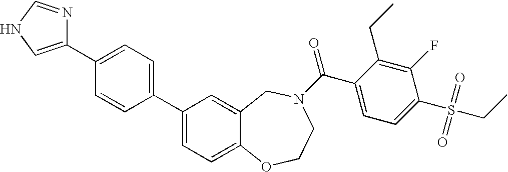 | 4-{[2-ethyl-4-(ethylsulfonyl)-3- fluorophenyl]carbonyl}-7-[4- (1H-imidazol-4-yl)phenyl]- 2,3,4,5-tetrahydro-1,4- benzoxazepine |
| 16 | 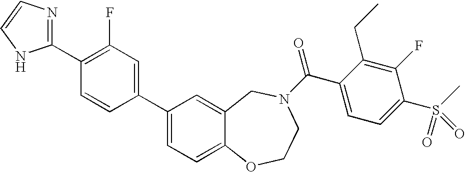 | 4-{[2-ethyl-3-fluoro-4- (methylsulfonyl)phenyl]carbonyl}- 7-[3-fluoro-4-(1H-imidazol-2- yl)phenyl]-2,3,4,5-tetrahydro- 1,4-benzoxazepine |
| 17 | 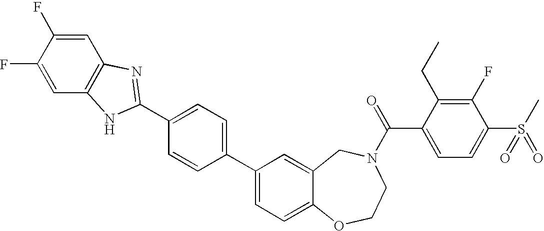 | 7-[4-(5,6-difluoro-1H- benzimidazol-2-yl)phenyl]-4- {[2-ethyl-3-fluoro-4- (methylsulfonyl)phenyl]carbonyl}- 2,3,4,5-tetrahydro-1,4- benzoxazepine |
| 18 | 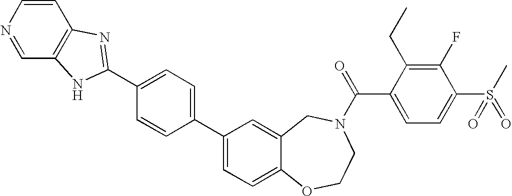 | 4-{[2-ethyl-3-fluoro-4- (methylsulfonyl)phenyl]carbonyl}- 7-[4-(3H-imidazo[4,5- c]pyridin-2-yl)phenyl]-2,3,4,5- tetrahydro-1,4-benzoxazepine |
| 19 | 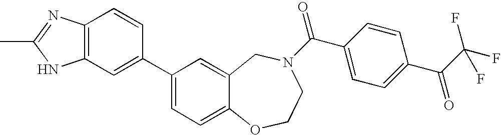 | 2,2,2-trifluoro-l-(4-{[7-(2- methyl-1H-benzimidazol-6-yl)- 2,3-dihydro-l,4-benzoxazepin- 4(5H)- yl]carbonyl}phenyl)ethanone |
| 20 | 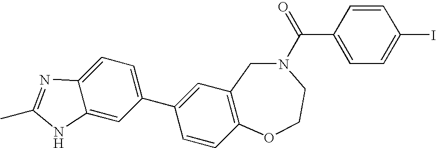 | 4-[(4-iodophenyl)carbonyl]-7- (2-methyl-1H-benzimidazol-6- yl)-2,3,4,5-tetrahydro-1,4- benzoxazepine |
| 21 | 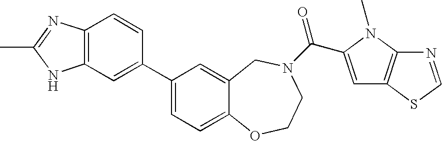 | 7-(2-methyl-1H-benzimidazol-6- yl)-4-[(4-methyl-4H-pyrrolo[2,3- d][1,3]thiazol-5-yl)carbonyl]- 2,3,4,5-tetrahydro-1,4- benzoxazepine |
| 22 | 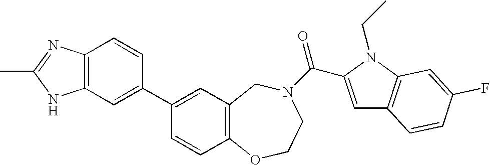 | 4-[(1-ethyl-6-fluoro-1H-indol-2- yl)carbonyl]-7-(2-methyl-1H- benzimidazol-6-yl)-2,3,4,5- tetrahydro-1,4-benzoxazepine |
| 23 | 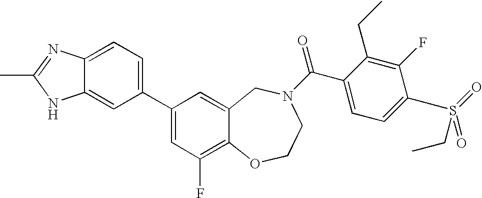 | 4-{[2-ethyl-4-(ethylsulfonyl)-3- fluorophenyl]carbonyl}-9- fluoro-7-(2-methyl-1H- benzimidazol-6-yl)-2,3,4,5- tetrahydro-1,4-benzoxazepine |
| 24 | 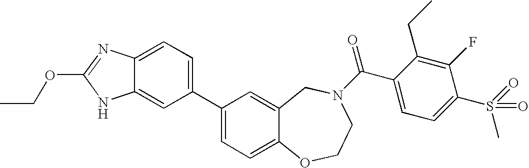 | 4-{[2-ethyl-3-fluoro-4- (methylsulfonyl)phenyl]carbonyl}- 7-[2-(ethyloxy)-1H- benzimidazol-6-yl]-2,3,4,5- tetrahydro-1,4-benzoxazepine |
| 25 | 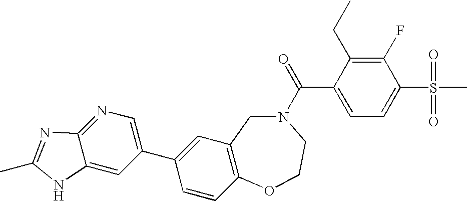 | 4-{[2-ethyl-3-fluoro-4- (methylsulfonyl)phenyl]carbonyl}- 7-(2-methyl-1H-imidazo[4,5- b]pyridin-6-yl)-2,3,4,5- tetrahydro-1,4-benzoxazepine |
| 26 | 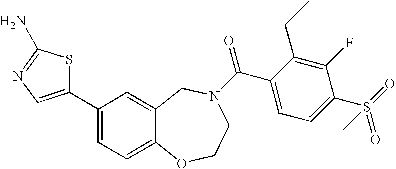 | 5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-amine |
| 27 | 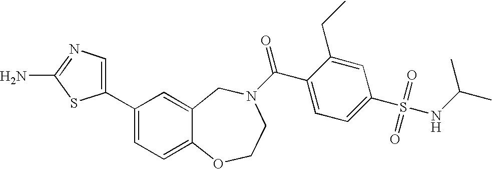 | 4-{[7-(2-amino-1,3-thiazol-5- yl)-2,3-dihydro-1,4- benzoxazepin-4(5H)- yl]carbonyl}-3-ethyl-N-(1- methylethyl)benzenesulfonamide |
| 28 | 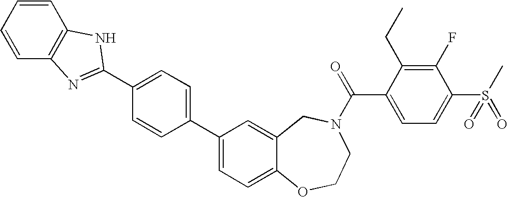 | 7-[4-(1H-benzimidazol-2- yl)phenyl]-4-{[2-ethyl-3-fluoro-4- (methylsulfonyl)phenyl]carbonyl}- 2,3,4,5-tetrahydro-1,4- benzoxazepine |
| 29 | 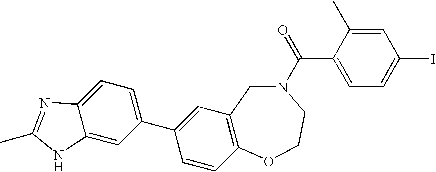 | 4-[(4-iodo-2- methylphenyl)carbonyl]-7-(2- methyl-1H-benzimidazol-6-yl)- 2,3,4,5-tetrahydro-1,4- benzoxazepine |
| 30 | 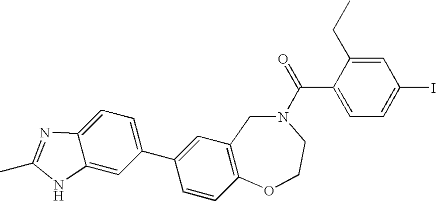 | 4-[(2-ethyl-4- iodophenyl)carbonyl]-7-(2- methyl-1H-benzimidazol-6-yl)- 2,3,4,5-tetrahydro-1,4- benzoxazepine |
| 31 | 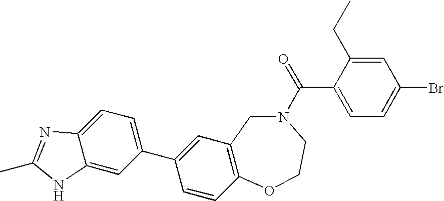 | 4-[(4-bromo-2- ethylphenyl)carbonyl]-7-(2- methyl-1H-benzimidazol-6-yl)- 2,3,4,5-tetrahydro-1,4- benzoxazepine |
| 32 | 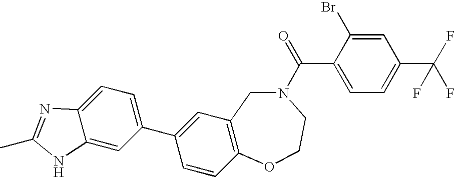 | 4-{[2-bromo-4- (trifluoromethyl)phenyl] carbonyl}-7-(2-methyl-1H- benzimidazol-6-yl)-2,3,4,5- tetrahydro-1,4-benzoxazepine |
| 33 | 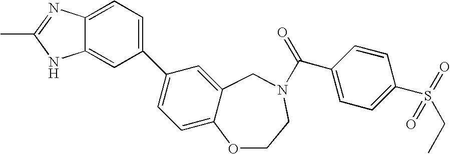 | 4-{[4- (ethylsulfonyl)phenyl]carbonyl}- 7-(2-methyl-1H-benzimidazol-6- yl)-2,3,4,5-tetrahydro-1,4- benzoxazepine |
| 34 | 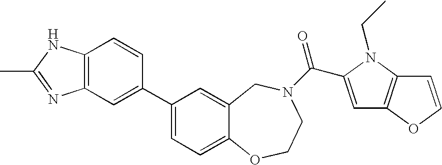 | 4-[(4-ethyl-4H-furo[3,2- b]pyrrol-5-yl)carbonyl]-7-(2- methyl-1H-benzimidazol-5-yl)- 2,3,4,5-tetrahydro-1,4- benzoxazepine |
| 35 | 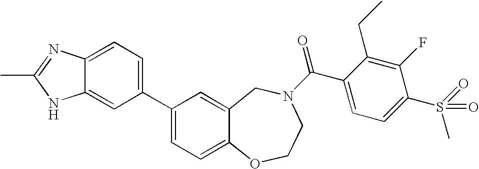 | 4-{[2-ethyl-3-fluoro-4- (methylsulfonyl)phenyl]carbonyl}- 7-(2-methyl-1H-benzimidazol- 6-yl)-2,3,4,5-tetrahydro-1,4- benzoxazepine |
| 36 | 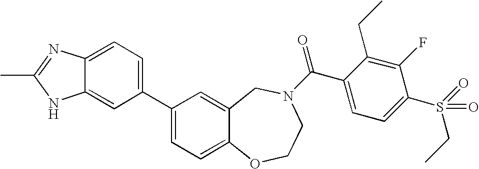 | 4-{[2-ethyl-4-(ethylsulfonyl)-3- fluorophenyl]carbonyl}-7-(2- methyl-1H-benzimidazol-6-yl)- 2,3,4,5-tetrahydro-1,4- benzoxazepine |
| 37 | 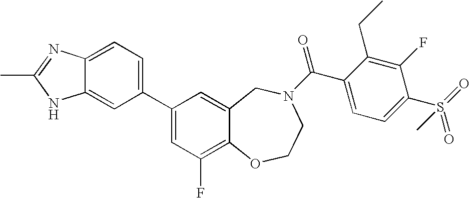 | 4-{[2-ethyl-3-fluoro-4- (methylsulfonyl)phenyl]carbonyl}- 9-fluoro-7-(2-methyl-1H- benzimidazol-6-yl)-2,3,4,5- tetrahydro-1,4-benzoxazepine |
| 38 | 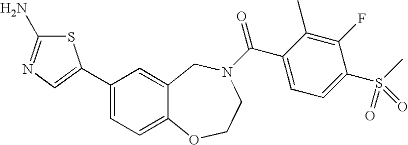 | 5-(4-{[3-fluoro-2-methyl-4- (methylsulfonyl)phenyl]carbonyl}- 2,3,4,5-tetrahydro-1,4- benzoxazepin-7-yl)-1,3-thiazol- 2-amine |
| 39 | 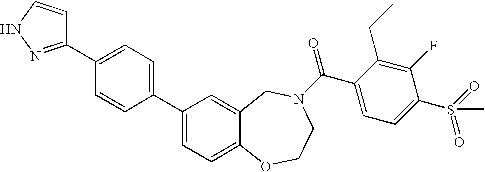 | 4-{[2-ethyl-3-fluoro-4- (methylsulfonyl)phenyl]carbonyl}- 7-[4-(1H-pyrazol-3- yl)phenyl]-2,3,4,5-tetrahydro- 1,4-benzoxazepine |
| 40 | 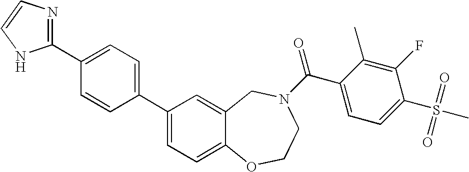 | 4-{[3-fluoro-2-methyl-4- (methylsulfonyl)phenyl]carbonyl}- 7-[4-(1H-imidazol-2- yl)phenyl]-2,3,4,5-tetrahydro- 1,4-benzoxazepine |
| 41 | 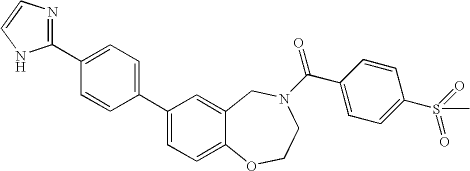 | 7-[4-(1H-imidazol-2-yl)phenyl]- 4-{[4- (methylsulfonyl)phenyl] carbonyl}-2,3,4,5-tetrahydro- 1,4-benzoxazepine |
| 42 | 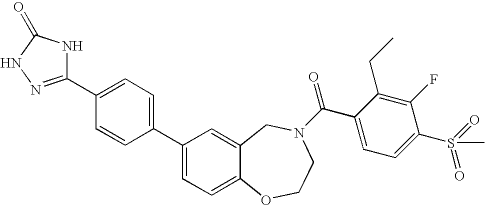 | 5-[4-(4-{[2-ethyl-3-fluoro-4- (methylsulfonyl)phenyl] carbonyl}-2,3,4,5-tetrahydro-1,4- benzoxazepin-7-yl)phenyl]-2,4- dihydro-3H-1,2,4-triazol-3-one |
| 43 | 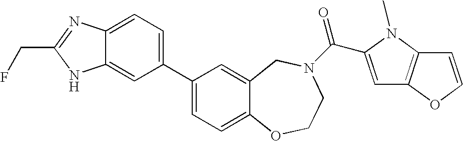 | 7-[2-(fluoromethyl)-1H- benzimidazol-6-yl]-4-[(4- methyl-4H-furo[3,2-b]pyrrol-5- yl)carbonyl]-2,3.4,5-tetrahydro- 1,4-benzoxazepine |
| 44 | 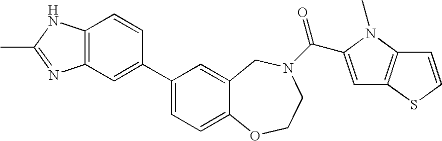 | 7-(2-methyl-1H-benzimidazol-5- yl)-4-[(4-methyl-4H-thieno[3,2- b]pyrrol-5-yl)carbonyl]-2,3,4,5- tetrahydro-1,4-benzoxazepine |
| 45 | 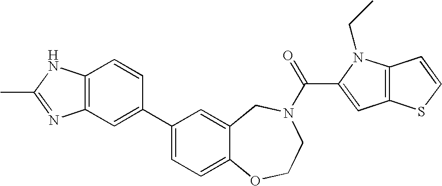 | 4-[(4-ethyl-4H-thieno[3,2- b]pyrrol-5-yl)carbonyl]-7-(2- methyl-1H-benzimidazol-5-yl)- 2,3,4,5-tetrahydro-1,4- benzoxazepine |
| 46 | 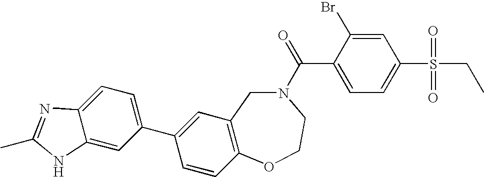 | 4-{[2-bromo-4- (ethylsulfonyl)phenyl]carbonyl}- 7-(2-methyl-1H-benzimidazol-6- yl)-2,3,4,5-tetrahydro-1,4- benzoxazepine |
| 47 | 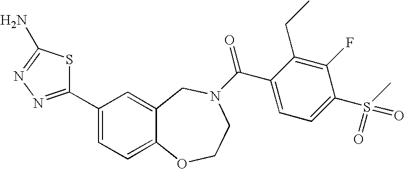 | 5-(4-{[2-ethyl-3-fluoro-4- (methylsulfonyl)phenyl]carbonyl}- 2,3,4,5-tetrahydro-1,4- benzoxazepin-7-yl)-1,3,4- thiadiazol-2-amine |
| 48 | 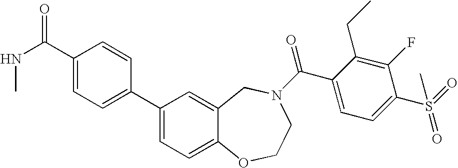 | 4-(4-{[2-ethyl-3-fluoro-4- (methylsulfonyl)phenyl]carbonyl}- 2,3,4,5-tetrahydro-1,4- benzoxazepin-7-yl)-N- methylbenzamide |
| 49 | 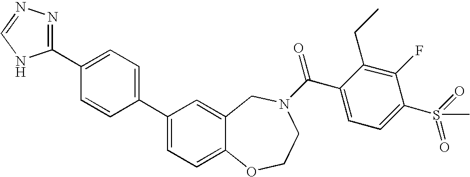 | 4-{[2-ethyl-3-fluoro-4- (methylsulfonyl)phenyl]carbonyl}- 7-[4-(4H-1,2,4-triazol-3- yl)phenyl]-2,3,4,5-tetrahydro- 1,4-benzoxazepine |
| 50 | 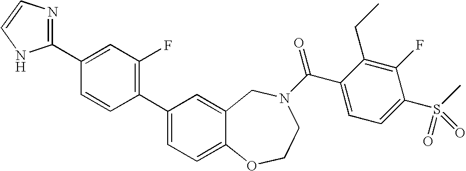 | 4-{[2-ethyl-3-fluoro-4- (methylsulfonyl)phenyl]carbonyl}- 7-[2-fluoro-4-(1H-imidazol-2- yl)phenyl]-2,3,4,5-tetrahydro- 1,4-benzoxazepine |
| 51 | 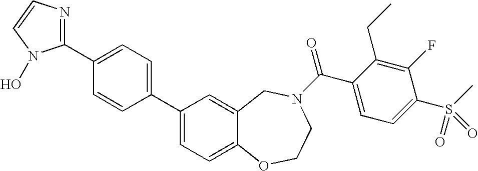 | 2-[4-(4-{[2-ethyl-3-fluoro-4- (methylsulfonyl)phenyl]carbonyl}- 2,3,4,5-tetrahydro-1,4- benzoxazepin-7-yl)phenyl]-1H- imidazol-1-ol |
| 52 | 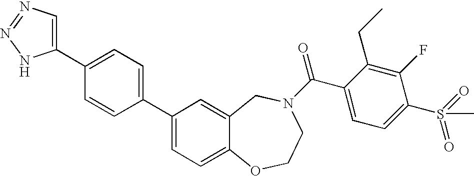 | 4-{[2-ethyl-3-fluoro-4- (methylsulfonyl)phenyl]carbonyl}- 7-[4-(1H-1,2,3-triazol-5- yl)phenyl]-2,3,4,5-tetrahydro- 1,4-benzoxazepine |
| 53 | 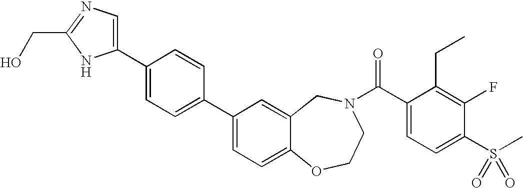 | {5-[4-(4-{[2-ethyl-3-fluoro-4- (methylsulfonyl)phenyl]carbonyl}- 2,3,4,5-tetrahydro-1,4- benzoxazepin-7-yl)phenyl]-1H- imidazol-2-yl}methanol |
| 54 | 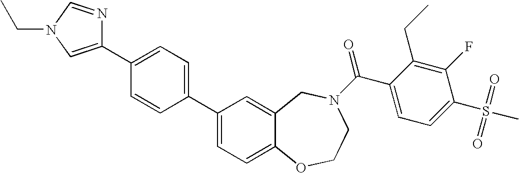 | 4-{[2-ethyl-3-fluoro-4- (methylsulfonyl)phenyl]carbonyl}- 7-[4-(1-ethyl-1H-imidazol-4- yl)phenyl]-2,3,4,5-tetrahydro- 1,4-benzoxazepine |
| 55 | 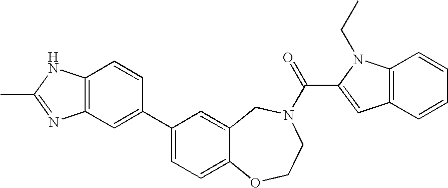 | 4-[(1-ethyl-1H-indol-2- yl)carbonyl]-7-(2-methyl-1H- benzimidazol-5-yl)-2,3,4,5- tctrahydro-1,4-benzoxazepine |
| 56 | 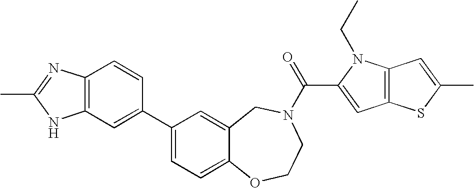 | 4-[(4-ethyl-2-methyl-4H- thieno[3,2-b]pyrrol-5- yl)carbonyl]-7-(2-methyl-1H- benzimidazol-6-yl)-2,3,4,5- tetrahydro-1,4-benzoxazepine |
| 57 | 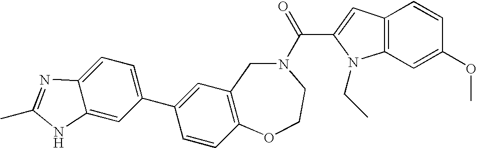 | 4-{[1-ethyl-6-(methyloxy)-1H- indol-2-yl]carbonyl}-7-(2- methyl-1H-benzimidazol-6-yl)- 2,3,4,5-tetrahydro-1,4- benzoxazepine |
| 58 | 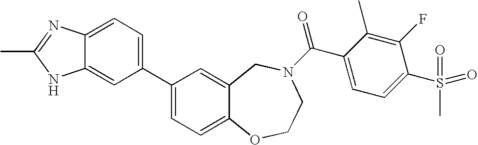 | 4-{[3-fluoro-2-methyl-4- (methylsulfonyl)phenyl]carbonyl}- 7-(2-methyl-1H-benzimidazol- 6-yl)-2,3,4,5-tetrahydro-l,4- benzoxazepine |
| 59 | 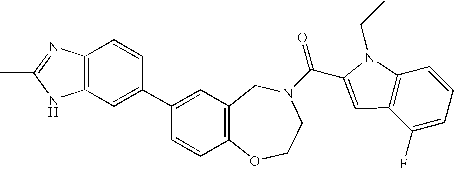 | 4-[(1-ethyl-4-fluoro-1H-indol-2- yl)carbonyl]-7-(2-methyl-1H- benzimidazol-6-yl)-2,3,4,5- tetrahydro-1,4-benzoxazepine |
| 60 | 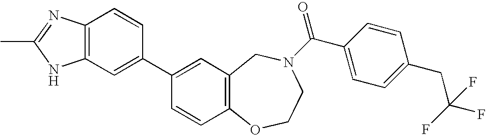 | 7-(2-methyl-1H-benzimidazol-6- yl)-4-{[4-(2,2,2- trifluoroethyl)phenyl]carbonyl}- 2,3,4,5-tetrahydro-1,4- benzoxazepine |
| 61 | 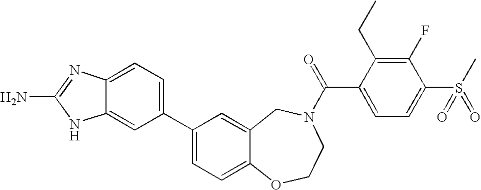 | 6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2-amine |
| 62 | 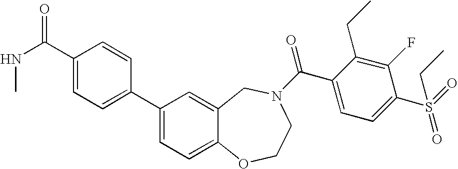 | 4-(4-{[2-ethyl-4-(ethylsulfonyl)- 3-fluorophenyl]carbonyl}- 2,3,4,5-tetrahydro-1,4- benzoxazepin-7-yl)-N- methylbenzamide |
| 63 | 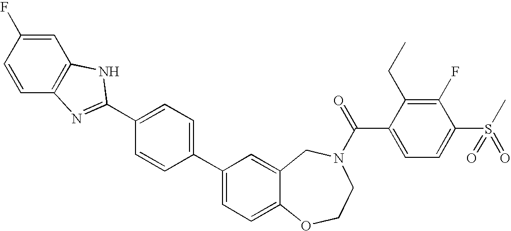 | 4-{[2-ethyl-3-fluoro-4- (methylsulfonyl)phenyl]carbonyl}- 7-[4-(6-fluoro-1H- benzimidazol-2-yl)phenyl]- 2,3,4,5-tetrahydro-1,4- benzoxazepine |
| 64 | 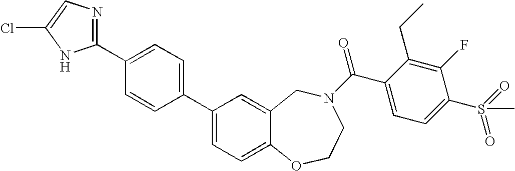 | 7-[4-(5-chloro-1H-imidazol-2- yl)phenyl]-4-{[2-ethyl-3-fluoro-4- (methylsulfonyl)phenyl]carbonyl}- 2,3,4,5-tetrahydro-1,4- benzoxazepine |
| 65 | 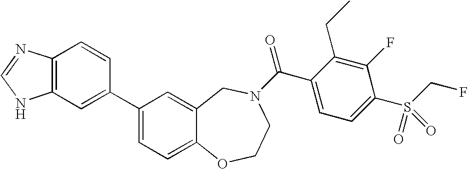 | 7-(1H-benzimidazol-6-yl)-4-({2- ethyl-3-fluoro-4- [(fluoromethyl)sulfonyl]phenyl} carbonyl)-2,3,4,5-tetrahydro-1,4- benzoxazepine |
| 66 | 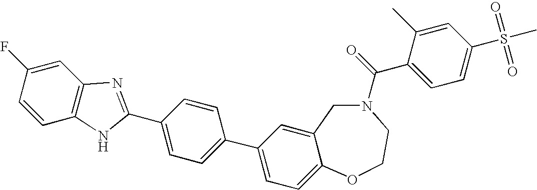 | 7-[4-(5-fluoro-1H-benzimidazol- 2-yl)phenyl]-4-{[2-methyl-4- (methylsulfonyl)phenyl]carbonyl}- 2,3,4,5-tetrahydro-1,4- benzoxazepine |
| 67 | 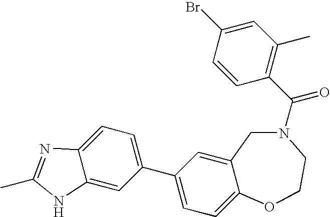 | 4-[(4-bromo-2- methylphenyl)carbonyl]-7-(2- methyl-1H-benzimidazol-6-yl)- 2,3,4,5-tetrahydro-1,4- benzoxazepine |
| 68 | 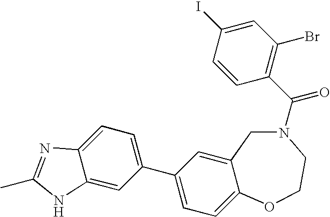 | 4-[(2-bromo-4- iodophenyl)carbonyl]-7-(2- methyl-1H-benzimidazol-6-yl)- 2,3,4,5-tetrahydro-1,4- benzoxazepine |
| 69 | 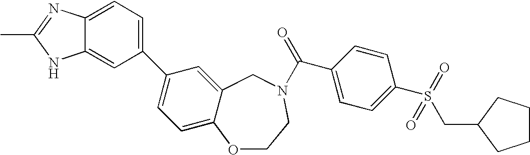 | 4-({4- [(cyclopentylmethyl)sulfonyl] phenyl}carbonyl)-7-(2-methyl-1H- benzimidazol-6-yl)-2,3,4,5- tetrahydro-1,4-benzoxazepine |
| 70 | 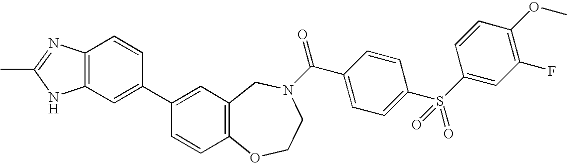 | 4-[(4-{[3-fluoro-4- (methyloxy)phenyl]sulfonyl}phenyl) carbonyl]-7-(2-methyl-1H- benzimidazol-6-yl)-2,3,4,5- tetrahydro-1,4-benzoxazepine |
| 71 | 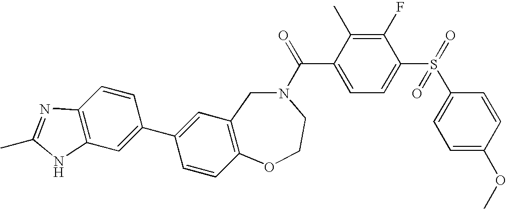 | 4-[(3-fluoro-2-methyl-4-{[4- (methyloxy)phenyl]sulfonyl}phenyl) carbonyl]-7-(2-methyl-1H- benzimidazol-6-yl)-2,3,4,5- tetrahydro-1,4-benzoxazepine |
| 72 | 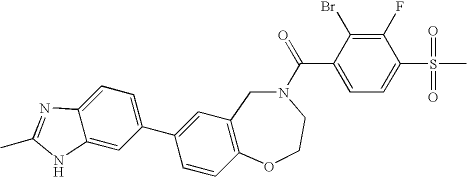 | 4-{[2-bromo-3-fluoro-4- (methylsulfonyl)phenyl]carbonyl}- 7-(2-methyl-1H-benzimidazol- 6-yl)-2,3,4,5-tetrahydro-1,4- benzoxazepine |
| 73 | 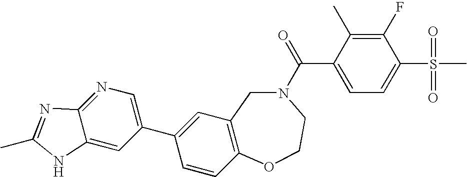 | 4-{[3-fluoro-2-methyl-4- (methylsulfonyl)phenyl]carbonyl}- 7-(2-methyl-1H-imidazo[4,5- b]pyridin-6-yl)-2,3,4,5- tetrahydro-1,4-benzoxazepine |
| 74 | 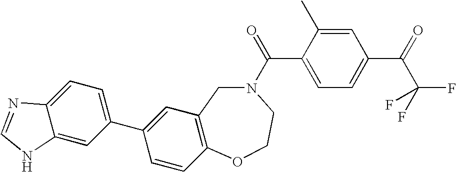 | 1-(4-{[7-(1H-benzimidazol-6- yl)-2,3-dihydro-1,4- benzoxazepin-4(5H)- yl]carbonyl}-3-methylphenyl)- 2,2,2-trifluoroethanone |
| 75 | 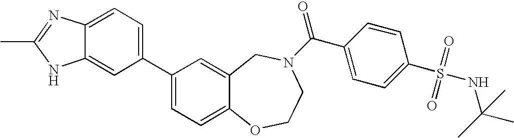 | N-(1,1-dimethylethyl)-4-{[7-(2- methyl-1H-benzimidazol-6-yl)- 2,3-dihydro-1,4-benzoxazepin- 4(5H)- yl]carbonyl}-benzenesulfonamide |
| 76 | 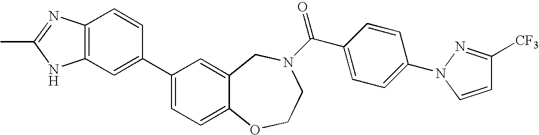 | 7-(2-methyl-1H-benzimidazol-6- yl)-4-({4-[3-(trifluoromethyl)- 1H-pyrazol-1- yl]phenyl}carbonyl)-2,3,4,5- tetrahydro-1,4-benzoxazepine |
| 77 | 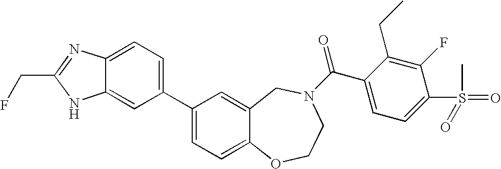 | 4-{[2-ethyl-3-fluoro-4- (methylsulfonyl)phenyl]carbonyl}- 7-[2-(fluoromethyl)-1H- benzimidazol-6-yl]-2,3,4,5- tetrahydro-1,4-benzoxazepine |
| 78 | 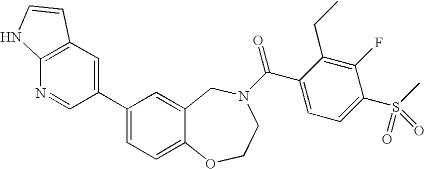 | 4-{[2-ethyl-3-fluoro-4- (methylsulfonyl)phenyl]carbonyl}- 7-(1H-pyrrolo[2,3-b]pyridin-5- yl)-2,3,4,5-tetrahydro-1,4- benzoxazepine |
| 79 | 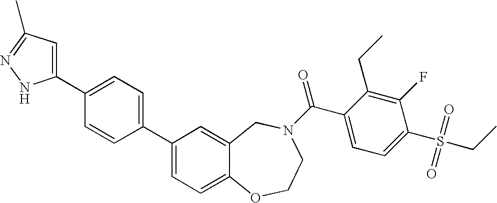 | 4-{[2-ethyl-4-(ethylsulfonyl)-3- fluorophenyl]carbonyl}-7-[4-(3- methyl-1H-pyrazol-5- yl)phenyl]-2,3,4,5-tetrahydro- 1,4-benzoxazepine |
| 80 | 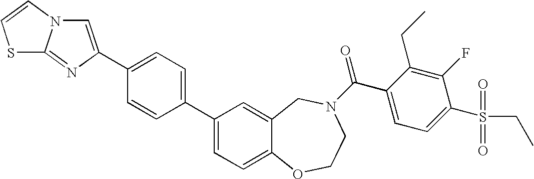 | 4-{[2-ethyl-4-(ethylsulfonyl)-3- fluoropheny]]carbonyl}-7-(4- imidazo[2,1-b][1,3]thiazol-6- ylphenyl)-2,3,4,5-tetrahydro-1,4- benzoxazepine |
| 81 | 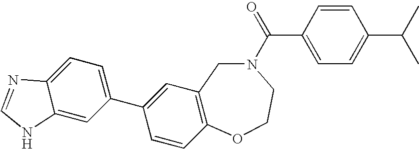 | 7-(1H-benzimidazol-6-yl)-4-{[4-(1- methylethyl)phenyl]carbonyl}- 2,3,4,5-tetrahydro-1,4- benzoxazepine |
| 82 | 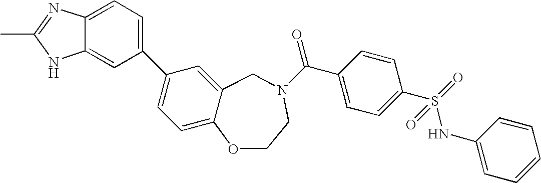 | 4-{[7-(2-methyl-1H- benzimidazol-6-yl)-2,3-dihydro- 1,4-benzoxazepin-4(5H)- yl]carbony]}-N- phenylbenzenesulfonamide |
| 83 | 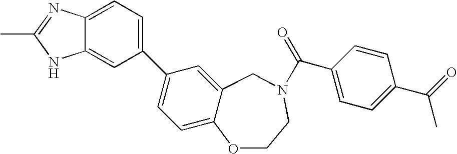 | l-(4-{[7-(2-methyl-1H- benzimidazol-6-yl)-2,3-dihydro- 1,4-benzoxazepin-4(5H)- yl]carbonyl}phenyl)ethanone |
| 84 | 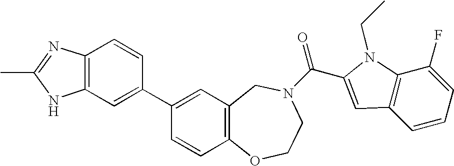 | 4-[(1-ethyl-7-fluoro-1H-indol-2- yl)carbonyl]-7-(2-methyl-1H- benzimidazol-6-yl)-2,3,4,5- tetrahydro-1,4-benzoxazepine |
| 85 | 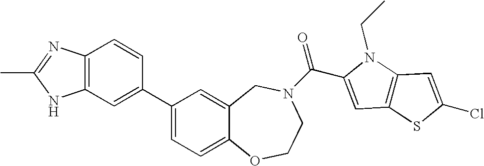 | 4-[(2-chloro-4-ethyl-4H- thieno[3,2-b]pyrrol-5- yl)carbonyl]-7-(2-methyl-1H- benzimidazol-6-yl)-2,3,4,5- tetrahydro-1,4-benzoxazepine |
| 86 | 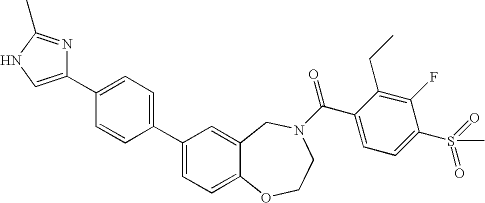 | 4-{[2-ethyl-3-fluoro-4- (methylsulfonyl)phenyl]carbonyl}- 7-[4-(2-methyl-1H-imidazol-4- yl)phenyl]-2,3,4,5-tetrahydro- 1,4-benzoxazepine |
| 87 | 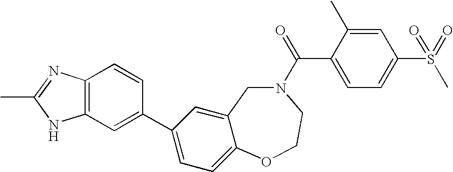 | 7-(2-methyl-1H-benzimidazol-6- yl)-4-{[2-methyl-4- (methylsulfonyl)phenyl]carbonyl}- 2,3,4,5-tetrahydro-1,4- benzoxazepine |
| 88 | 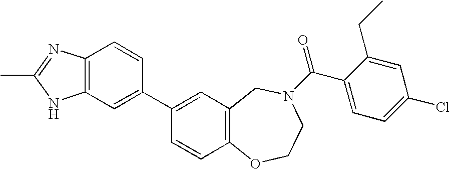 | 4-[(4-chloro-2- ethylphenyl)carbonyl]-7-(2- methyl-1H-benzimidazol-6-yl)- 2,3,4,5-tetrahydro-1,4- benzoxazepine |
| 89 | 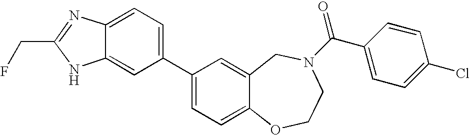 | 4-[(4-chlorophenyl)carbonyl]-7- [2-(fluoromethyl)-1H- benzimidazol-6-yl]-2,3,4,5- tetrahydro-1,4-benzoxazepine |
| 90 | 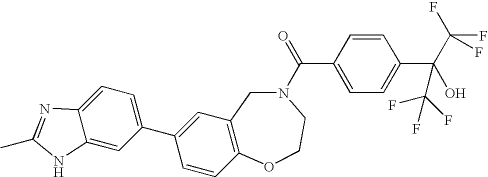 | 1,1,1,3,3,3-hexafluoro-2-(4-{[7- (2-methyl-1H-benzimidazol-6- yl)-2,3-dihydro-1,4- benzoxazepin-4(5H)- yl]carbonyl}phenyl)propan-2-ol |
| 91 | 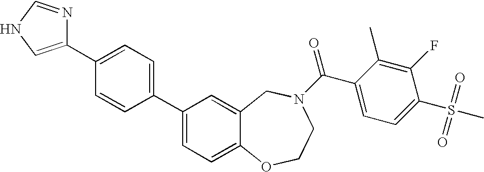 | 4-{[3-fluoro-2-methyl-4- (methylsulfonyl)phenyl]carbonyl}- 7-[4-(1H-imidazol-4- yl)phenyl]-2,3,4,5-tetrahydro- 1,4-benzoxazepine |
| 92 | 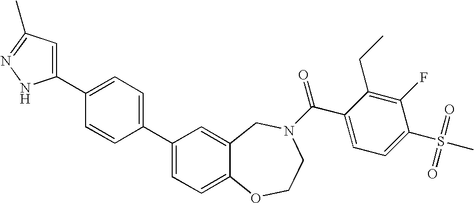 | 4-{[2-ethyl-3-fluoro-4- (methylsulfonyl)phenyl]carbonyl}- 7-[4-(3-methyl-1H-pyrazol-5- yl)phenyl]-2,3,4,5-tetrahydro- 1,4-benzoxazepine |
| 93 | 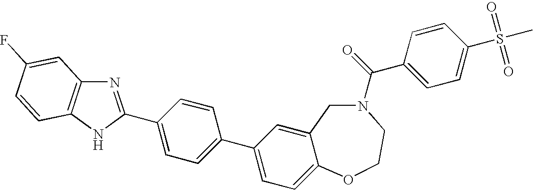 | 7-[4-(5-fluoro-1H-benzimidazol- 2-yl)phenyl]-4-{[4- (methylsulfonyl)phenyl]carbonyl}- 2,3,4,5-tetrahydro-1,4- benzoxazepine |
| 94 | 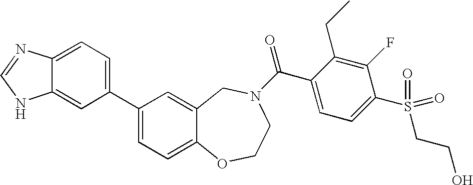 | 2-[(4-{[7-(1H-benzimidazol-6- yl)-2,3-dihydro-1,4- benzoxazepin-4(5H)- yl]carbonyl}-3-ethyl-2- fluorophenyl)sulfonyl]ethanol |
| 95 | 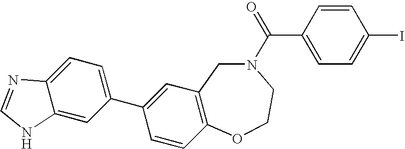 | 7-(1H-benzimidazol-6-yl)-4-[(4- iodophenyl)carbonyl]-2,3,4,5- tetrahydro-1,4-benzoxazepine |
| 96 | 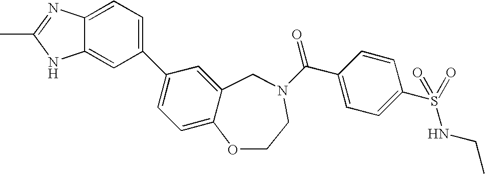 | N-ethyl-4-{[7-(2-methyl-1H- benzimidazol-6-yl)-2,3-dihydro- 1,4-benzoxazepin-4(5H)- yl]carbonyl}benzenesulfonamide |
| 97 | 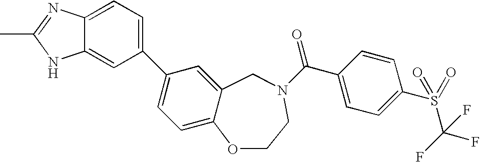 | 7-(2-methyl-1H-benzimidazol-6- yl)-4-({4- [(trifluoromethyl)sulfonyl]phenyl} carbonyl)-2,3,4,5-tetrahydro- 1,4-benzoxazepine |
| 98 | 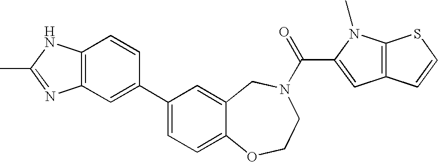 | 7-(2-methyl-1H-benzimidazol-5- yl)-4-[(6-methyl-6H-thieno[2,3- b]pyrrol-5-yl)carbonyl]s2,3,4,5- tetrahydro-1,4-benzoxazepine |
| 99 | 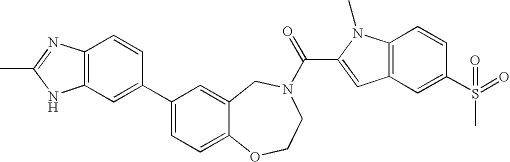 | 7-(2-methyl-1H-benzimidazol-6- yl)-4-{[1-methyl-5- (methylsulfonyl)-1H-indol-2- yl]carbonyl}-2,3,4,5-tetrahydro- 1,4-benzoxazepine |
| 100 | 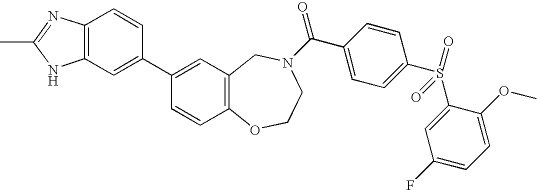 | 4-[(4-{[5-fluoro-2- (methyloxy)phenyl]sulfonyl}phenyl) carbonyl]-7-(2-methyl-1H- benzimidazol-6-yl)-2,3,4,5- tetrahydro-1,4-benzoxazepine |
| 101 | 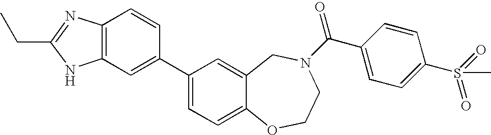 | 7-(2-ethyl-1H-benzimidazol-6- yl)-4-{[4- (methylsulfonyl)phenyl]carbonyl}- 2,3,4,5-tetrahydro-1,4- benzoxazepine |
| 102 | 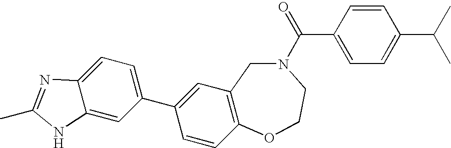 | 7-(2-methyl-1H-benzimidazol-6- yl)-4-{[4-(1- methylethyl)phenyl]carbonyl}- 2,3,4,5-tetrahydro-1,4- benzoxazepine |
| 103 | 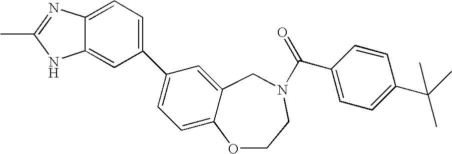 | 4-{[4-(1,1- dimethylethyl)phenyl]carbonyl}- 7-(2-methyl-1H-benzimidazol-6- yl)-2,3,4,5-tetrahydro-1,4- benzoxazepine |
| 104 | 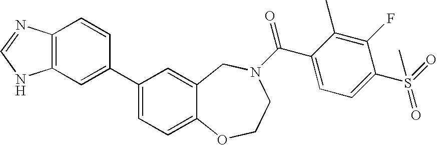 | 7-(1H-benzimidazol-6-yl)-4-{[3- fluoro-2-methyl-1-4- (methylsulfonyl)phenyl]carbonyl}- 2,3,4,5-tetrahydro-1,4- benzoxazepine |
| 105 | 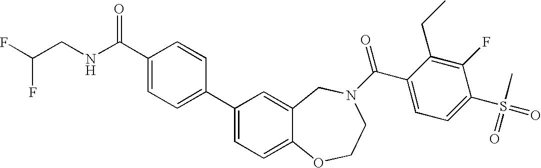 | N-(2,2-difluoroethyl)-4-(4-{[2- ethyl-3-fluoro-4- (methylsulfonyl)phenyl]carbonyl}- 2,3,4,5-tetrahydro-1,4- benzoxazepin-7-yl)benzamide |
| 106 | 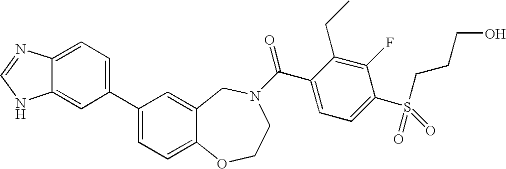 | 3-[(4-{[7-(1H-benzimidazol-6- yl)-2,3-dihydro-1,4- benzoxazepin-4(5H)- yl]carbonyl}-3-ethyl-2- fluorophenyl)sulfonyl]propan-1-ol |
| 107 | 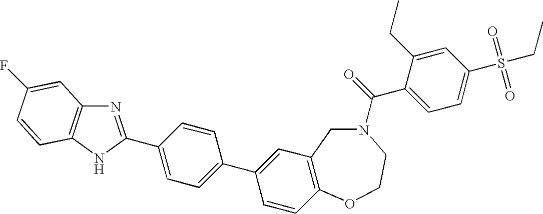 | 4-{[2-ethyl-4- (ethylsulfonyl)phenyl]carbonyl}- 7-[4-(5-fluoro-1H-benzimidazol- 2-yl)phenyl]-2,3,4,5-tetrahydro- 1,4-benzoxazepine |
| 108 | 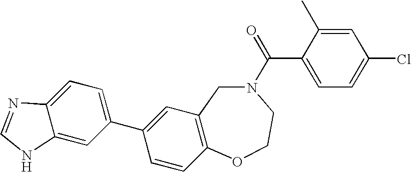 | 7-(1H-benzimidazol-6-yl)-4-[(4- chloro-2- methylphenyl)carbonyl]-2,3,4,5- tetrahydro-1,4-benzoxazepine |
| 109 | 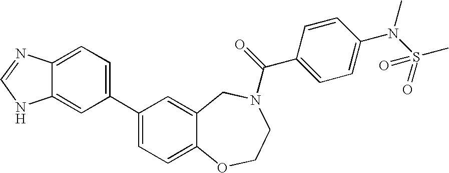 | N-(4-{[7-(1H-benzimidazol-6- yl)-2,3-dihydro-1,4- benzoxazepin-4(5H)- yl]carbonyl}phenyl)-N- methylmethanesulfonamide |
| 110 | 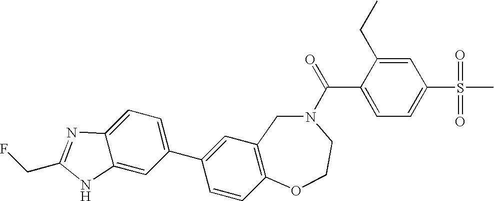 | 4-{[2-ethyl-4- (methylsulfonyl)phenyl]carbonyl}- 7-[2-(fluoromethyl)-1H- benzimidazol-6-yl]-2,3,4,5- tetrahydro-1,4-benzoxazepine |
| 111 | 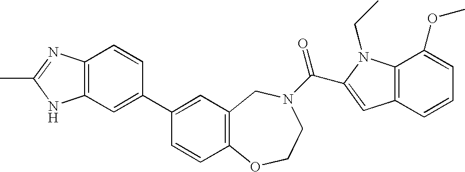 | 4-{[1-ethyl-7-(methyloxy)-1H- indol-2-yl]carbonyl}-7-(2- methyl-1H-benzimidazol-6-yl)- 2,3,4,5-tetrahydro-1,4- benzoxazepine |
| 112 | 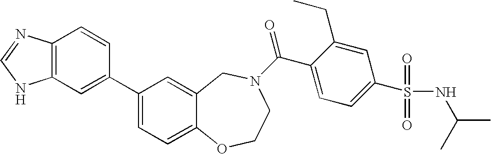 | 4-{[7-(1H-benzimidazol-6-yl)- 2,3-dihydro-1,4-benzoxazepin- 4(5H)-yl]carbonyl}-3-ethyl-N-(1- methylethyl)benzenesulfonamide |
| 113 | 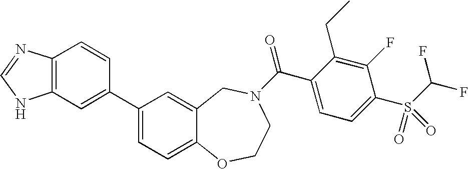 | 7-(1H-benzimidazol-6-yl)-4-({4- [(difluoromethyl)sulfonyl]-2- ethyl-3-fluorophenyl}carbonyl)- 2,3,4,5-tetrahydro-1,4- benzoxazepine |
| 114 | 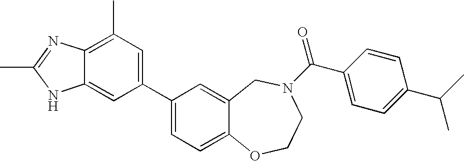 | 7-(2,4-dimethyl-1H- benzimidazol-6-yl)-4-{[4-(1- methylethyl)phenyl]carbonyl}- 2,3,4,5-tetrahydro-1,4- benzoxazepine |
| 115 | 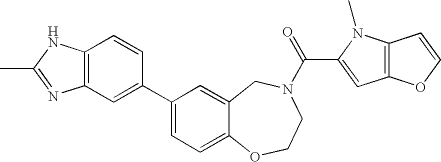 | 7-(2-methyl-1H-benzimidazol-5- yl)-4-[(4-methyl-4H-furo[3,2- b]pyrrol-5-yl)carbonyl]-2,3,4,5- tetrahydro-1,4-benzoxazepine |
| 116 | 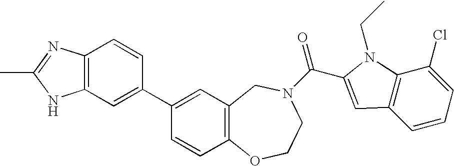 | 4-[(7-chloro-1-ethyl-1H-indol-2- yl)carbonyl]-7-(2-methyl-1H- benzimidazol-6-yl)-2,3,4,5- tetrahydro-1,4-benzoxazepine |
| 117 | 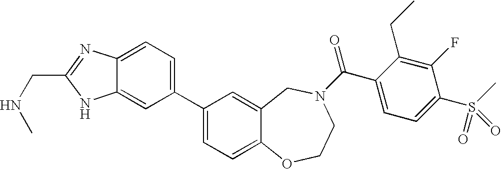 | l-[6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2-yl]-N- methylmethanamine |
| 118 | 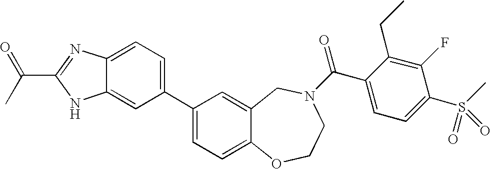 | l-[6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2-yl]ethanone |
| 119 | 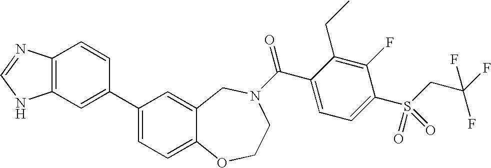 | 7-(1H-benzimidazol-6-yl)-4-((2- ethyl-3-fluoro-4-[(2,2,2- trifluoroethyl)sulfonyl]phenyl} carbonyl)-2,3,4,5-tetrahydro-1,4- benzoxazepine |
| 120 | 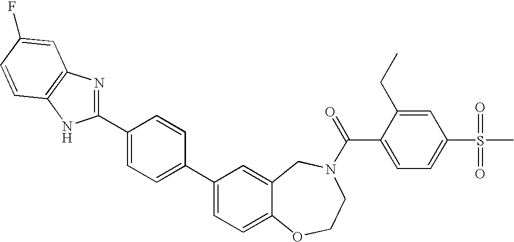 | 4-{[2-ethyl-4- (methylsulfonyl)phenyl]carbonyl}- 7-[4-(5-fluoro-1H- benzimidazol-2-yl)phenyl]- 2,3,4,5-tetrahydro-1,4- benzoxazepine |
| 121 | 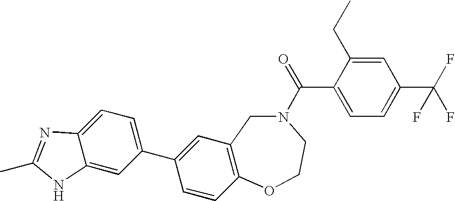 | 4-{[2-ethyl-4- (trifluoromethyl)phenyl]carbonyl}- 7-(2-methyl-1H- benzimidazol-6-yl)-2,3,4,5- tetrahydro-1,4-benzoxazepine |
| 122 | 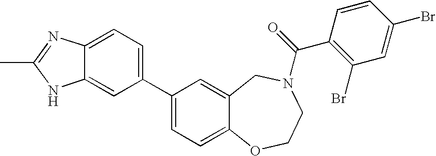 | 4-[(2,4- dibromophenyl)carbonyl]-7-(2- methyl-1H-benzimidazol-6-yl)- 2,3,4,5-tetrahydro-1,4- benzoxazepine |
| 123 | 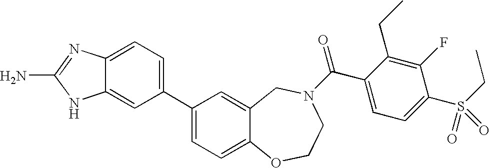 | 6-(4-{[2-ethy]-4-(ethylsulfonyl)- 3-fluorophenyl]carbonyl}- 2,3,4,5-tetrahydro-1,4- benzoxazepin-7-yl)-1H- benzimidazol-2-amine |
| 124 | 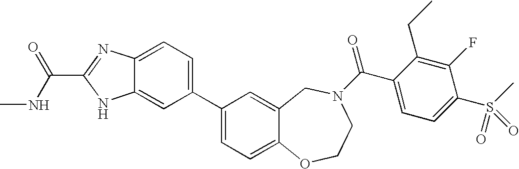 | 6-(4-{[2-ethyl-3-fluoro-4- (methylsulfonyl)phenyl]carbonyl}- 2,3,4,5-tetrahydro-1,4- benzoxazepin-7-yl)-N-methyl- 1H-benzimidazole-2- carboxamide |
| 125 | 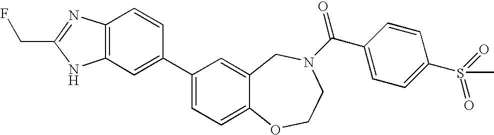 | 7-[2-(fluoromethyl)-1H- benzimidazol-6-yl]-4-{[4- (methylsulfonyl)phenyl]carbonyl}- 2,3,4,5-tetrahydro-1,4- benzoxazepine |
| 126 | 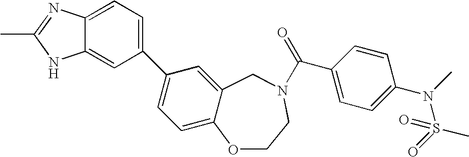 | N-methyl-N-(4-{[7-(2-methyl- 1H-benzimidazol-6-yl)-2,3- dihydro-1,4-benzoxazepin- 4(5H)-yl]carbonyl}phenyl) methanesulfonamide |
| 127 | 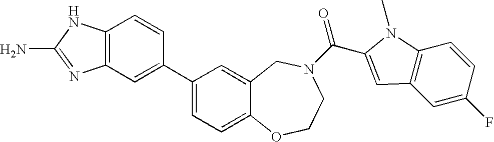 | 4-[(5-fluoro-1-methyl-1H-indol- 2-yl)carbonyl]-7-(2-methyl-1H- benzimidazol-5-yl)-2,3,4,5- tetrahydro-1,4-benzoxazepine |
| 128 | 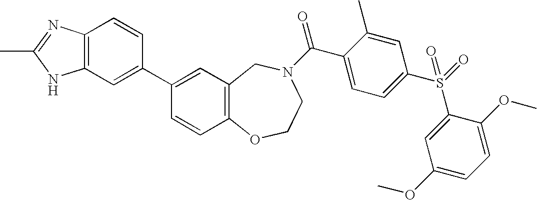 | 4-[(4-{[2,5- bis(methyloxy)phenyl]sulfonyl}- 2-methylphenyl)carbonyl]-7-(2- methyl-1H-benzimidazol-6-yl)- 2,3,4,5-tetrahydro-1,4- benzoxazepine |
| 129 | 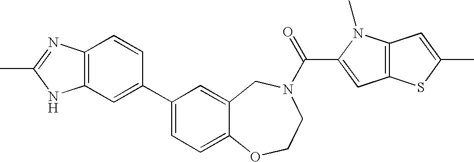 | 4-[(2,4-dimethyl-4H-thieno[3,2- b]pyrrol-5-yl)carbonyl]-7-(2- methyl-1H-benzimidazol-6-yl)- 2,3,4,5-tetrahydro-1,4- benzoxazepine |
| 130 | 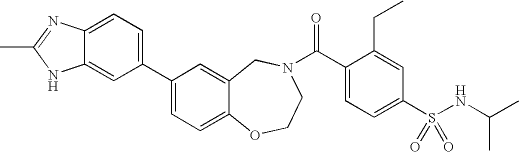 | 3-ethyl-4-{[7-(2-methyl-1H- benzimidazol-6-yl)-2,3-dihydro- 1,4-benzoxazepin-4(5H)- yl]carbonyl}-N-(1- methylethyl)benzenesulfonamide |
| 131 | 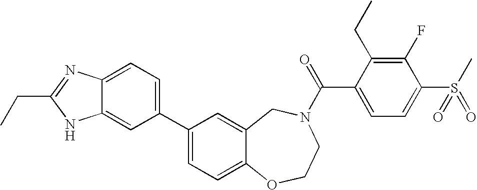 | 7-(2-ethyl-1H-benzimidazol-6- yl)-4-{[2-ethyl-3-fluoro-4- (methylsulfonyl)phenyl]carbonyl}- 2,3,4,5-tetrahydro-1,4- benzoxazepine |
| 132 | 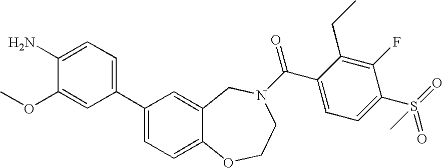 | 4-(4-{[2-ethyl-3-fluoro-4- (methylsulfonyl)phenyl]carbonyl}- 2,3,4,5-tetrahydro-1,4- benzoxazepin-7-yl)-2- (methyloxy)aniline |
| 133 | 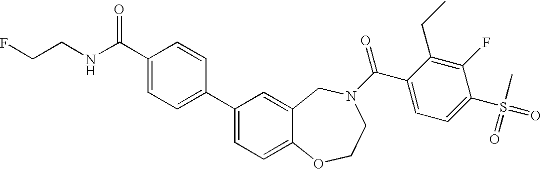 | 4-(4-{[2-ethyl-3-fluoro-4- (methylsulfonyl)phenyl]carbonyl}- 2,3,4,5-tetrahydro-1,4- benzoxazepin-7-yl)-N-(2- fluoroethyl)benzamide |
| 134 | 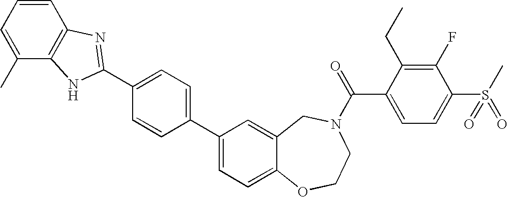 | 4-{[2-ethyl-3-fluoro-4- (methylsulfonyl)phenyl]carbonyl}- 7-[4-(7-methyl-1H- benzimidazol-2-yl)phenyl]- 2,3,4,5-tetrahydro-1,4- benzoxazepine |
| 135 | 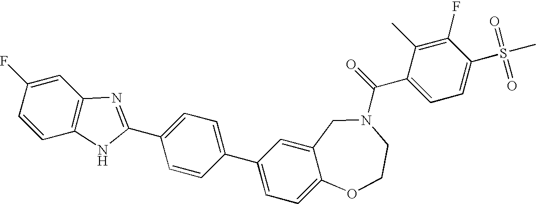 | 7-[4-(5-fluoro-1H-benzimidazol- 2-yl)phenyl]-4-{[3-fluoro-2- methyl-4- (methylsulfonyl)phenyl]carbonyl}- 2,3,4,5-tetrahydro-1,4- benzoxazepine |
| 136 | 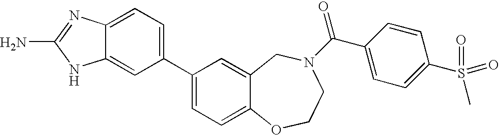 | 6-(4-{[4- (methylsulfonyl)phenyl]carbonyl}- 2,3,4,5-tetrahydro-1,4- benzoxazepin-7-yl)-1H- benzimidazol-2-amine |
| 137 | 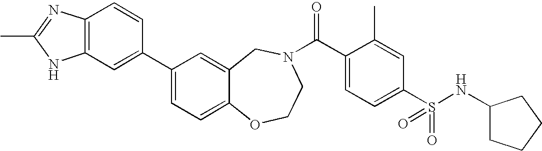 | N-cyclopentyl-3-methyl-4-{[7- (2-methyl-1H-benzimidazol-6- yl)-2,3-dihydro-1,4- benzoxazepin-4(5H)- yl]carbonyl}benzenesulfonamide |
| 138 | 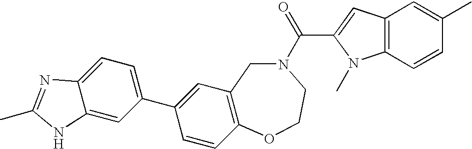 | 4-[(1,5-dimethyl-1H-indol-2- yl)carbonyl]-7-(2-methyl-1H- benzimidazol-6-yl)-2,3,4,5- tetrahydro-1,4-benzoxazepine |
| 139 | 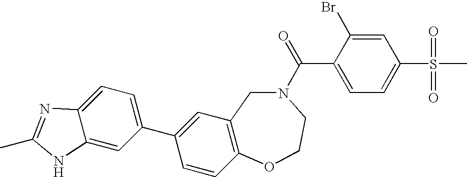 | 4-{[2-bromo-4- (methylsulfonyl)phenyl]carbonyl}- 7-(2-methyl-1H-benzimidazol- 6-yl)-2,3,4,5-tetrahydro-1,4- benzoxazepine |
| 140 | 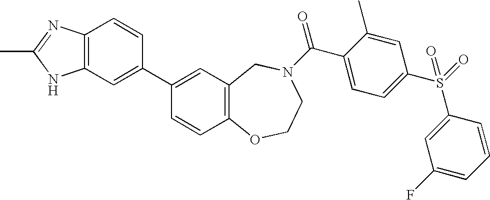 | 4-({4-[(3- fluorophenyl)sulfonyl]-2- methylphenyl}carbonyl)-7-(2- methyl-1H-benzimidazol-6-yl)- 2,3,4,5-tetrahydro-1,4- benzoxazepine |
| 141 | 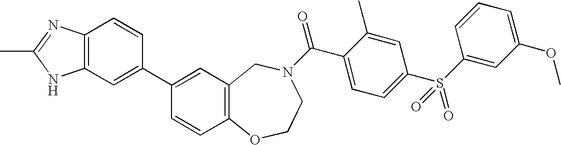 | 7-(2-methyl-1H-benzimidazol-6- yl)-4-[(2-methyl-4-{[3- (methyloxy)phenyl]sulfonyl}phenyl) carbonyl]-2,3,4,5-tetrahydro- 1,4-benzoxazepine |
| 142 | 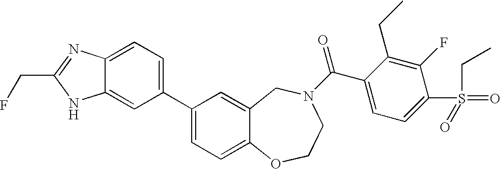 | 4-{[2-ethyl-4-(ethylsulfonyl)-3- fluorophenyl]carbonyl}-7-[2- (fluoromethyl)-1H- benzimidazol-6-yl]-2,3,4,5- tetrahydro-1,4-benzoxazepine |
| 143 | 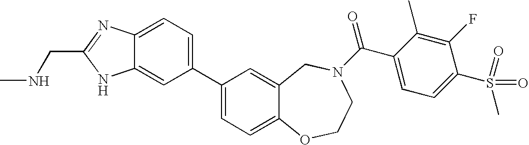 | l-[6-(4-{[3-fluoro-2-methyl-4- (methylsulfonyl)phenyl]carbonyl}- 2,3,4,5-tetrahydro-1,4- benzoxazepin-7-yl)-1H- benzimidazol-2-yl]-N- methylmethanamine |
| 144 | 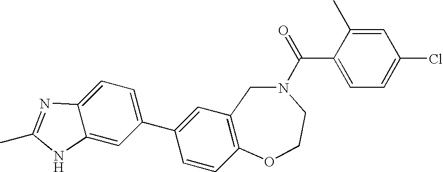 | 4-[(4-chloro-2- methylphenyl)carbonyl]-7-(2- methyl-1H-benzimidazol-6-yl)- 2,3,4,5-tetrahydro-1,4- benzoxazepine |
| 145 | 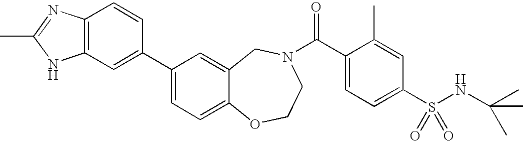 | N-(1,1-dimethylethyl)-3-methyl- 4-{[7-(2-methyl-1H- benzimidazol-6-yl)-2,3-dihydro- 1,4-benzoxazepin-4(5H)- yl]carbonyl}benzenesulfonamide |
| 146 | 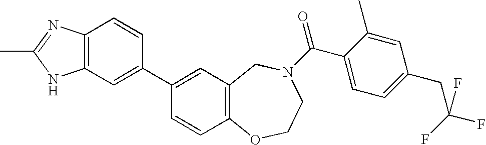 | 7-(2-methyl-1H-benzimidazol-6- yl)-4-{[2-methyl-4-(2,2,2- trifluoroethyl)phenyl]carbonyl}- 2,3,4,5-tetrahydro-1,4- benzoxazepine |
| 147 | 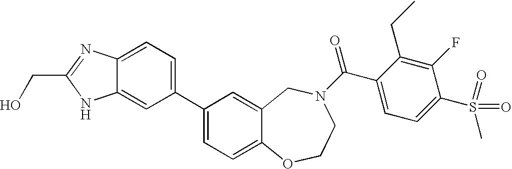 | [6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2-yl]methanol |
| 148 | 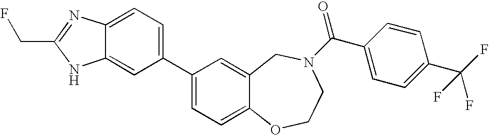 | 7-[2-(fluoromethyl)-1H- benzimidazol-6-yl]-4-{[4- (trifluoromethyl)phenyl]carbonyl}- 2,3,4,5-tetrahydro-1,4- benzoxazepine |
| 149 | 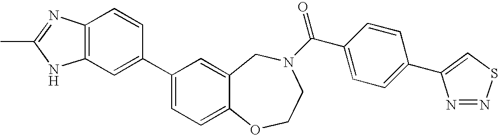 | 7-(2-methyl-1H-benzimidazol-6- yl)-4-{[4-(1,2,3-thiadiazol-4- yl)phenyl]carbonyl}-2,3,4,5- tetrahydro-1,4-benzoxazepine |
| 150 | 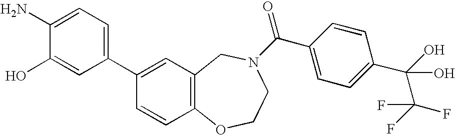 | 1-(4-{[7-(4-amino-3- hydroxyphenyl)-2,3-dihydro- 1,4-benzoxazepin-4(5H)- yl]carbonyl}phenyl)-2,2,2- trifluoroethane-1,1-diol |
| 151 | 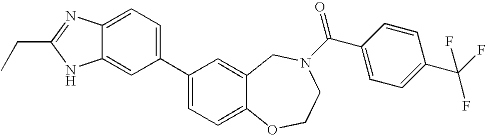 | 7-(2-ethyl-1H-benzimidazol-6- yl)-4-{[4- (trifluoromethyl)phenyl]carbonyl}- 2,3,4,5-tetrahydro-1,4- benzoxazepine |
| 152 | 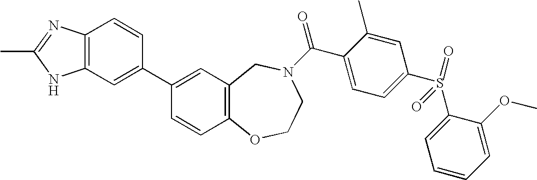 | 7-(2-methyl-1H-benzimidazol-6- yl)-4-[(2-methyl-4-{[2- (methyloxy)phenyl]sulfonyl}phenyl) carbonyl]-2,3,4,5-tetrahydro- 1,4-benzoxazepine |
| 153 | 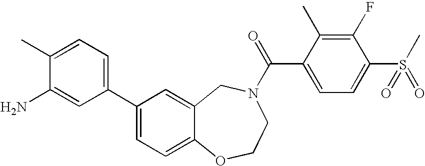 | 5-(4-{[3-fluoro-2-methyl-4- (methylsulfonyl)phenyl]carbonyl}- 2,3,4,5-tetrahydro-1,4- benzoxazepin-7-yl)-2- methylaniline |
| 154 | 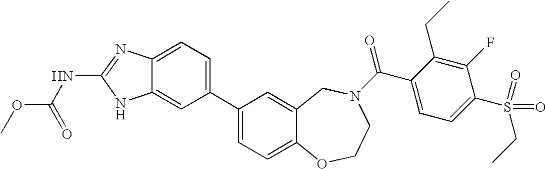 | methyl [6-(4-{[2-ethyl-4- (ethylsulfonyl)-3- fluorophenyl]carbonyl}-2,3,4,5- tetrahydro-1,4-benzoxazepin-7- yl)-1H-benzimidazol-2- yl]carbamate |
| 155 | 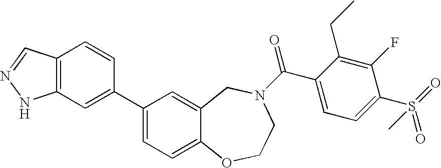 | 4-{[2-ethyl-3-fluoro-4- (methylsulfonyl)phenyl]carbonyl}- 7-(1H-indazol-6-yl)-2,3,4,5- tetrahydro-1,4-benzoxazepine |
| 156 | 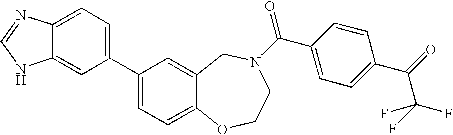 | 1-(4-{[7-(1H-benzimidazol-6- yl)-2,3-dihydro-1,4- benzoxazepin-4(5H)- yl]carbonyl}phenyl)-2,2,2- trifluoroethanone |
| 157 | 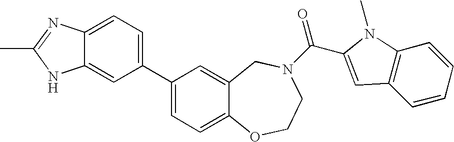 | 7-(2-methyl-1H-benzimidazol-6- yl)-4-[(1-methyl-1H-indol-2- yl)carbonyl]-2,3,4,5-tetrahydro- 1,4-benzoxazepine |
| 158 | 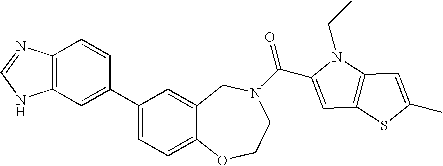 | 7-(1H-benzimidazol-6-yl)-4-[(4- ethy]-2-methy]-4H-thieno[3,2- b]pyrrol-5-yl)carbonyl]-2,3,4,5- tetrahydro-1,4-benzoxazepine |
| 159 | 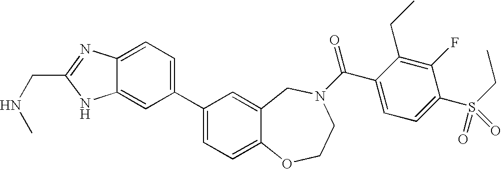 | l-[6-(4-{[2-ethyl-4- (ethylsulfonyl)-3- fluorophenyl]carbonyl}-2,3,4,5- tetrahydro-1,4-benzoxazepin-7- yl)-1H-benzimidazol-2-yl]-N- methylmethanamine |
| 160 | 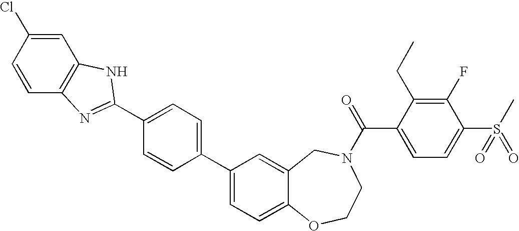 | 7-[4-(6-chloro-1H- benzimidazol-2-yl)phenyl]-4- {[2-ethyl-3-fluoro-4- (methylsulfonyl)phenyl]carbonyl}- 2,3,4,5-tetrahydro-1,4- benzoxazepine |
| 161 | 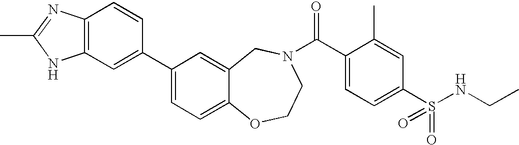 | N-ethyl-3-methyl-4-{[7-(2- methyl-1H-benzimidazol-6-yl)- 2,3-dihydro-1,4-benzoxazepin- 4(5H)- yl]carbonyl}benzenesulfonamide |
| 162 | 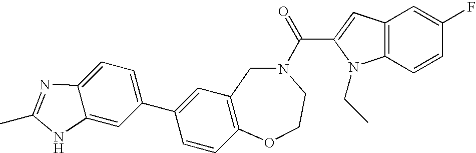 | 4-[(1-ethyl-5-fluoro-1H-indol-2- yl)carbonyl]-7-(2-methyl-1H- benzimidazol-6-yl)-2,3,4,5- tetrahydro-1,4-benzoxazepine |
| 163 | 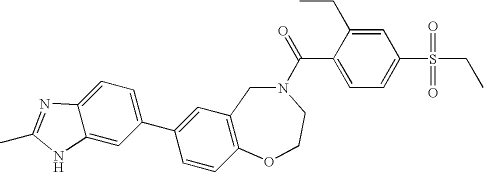 | 4-{[2-ethyl-4- (ethylsulfonyl)phenyl]carbonyl}- 7-(2-methyl-1H-benzimidazol-6- yl)-2,3,4,5-tetrahydro-1,4- benzoxazepine |
| 164 | 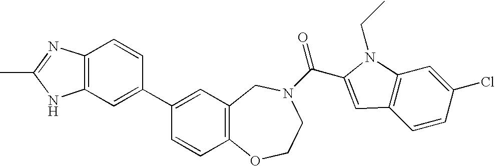 | 4-[(6-chloro-1-ethyl-1H-indol-2- yl)carbonyl]-7-(2-methyl-1H- benzimidazol-6-yl)-2,3,4,5- tetrahydro-1,4-benzoxazepine |
| 165 | 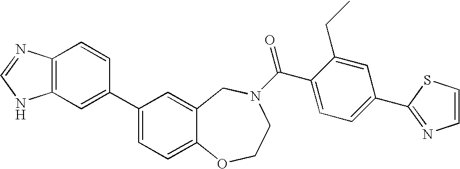 | 7-(1H-benzimidazol-6-yl)-4-{[2- ethyl-4-(1,3-thiazol-2- yl)phenyl]carbonyl}-2,3,4,5- tetrahydro-1,4-benzoxazepine |
| 166 | 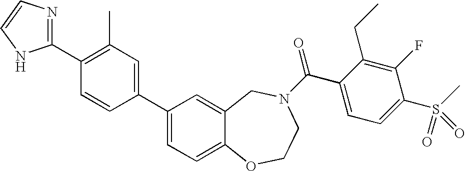 | 4-{[2-ethyl-3-fluoro-4- (methylsulfonyl)phenyl]carbonyl}- 7-[4-(1H-imidazol-2-yl)-3- methylphenyl]-2,3,4,5- tetrahydro-1,4-benzoxazepine |
| 167 | 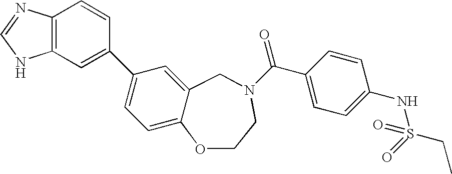 | N-(4-{[7-(1H-benzimidazol-6- yl)-2,3-dihydro-1,4- benzoxazepin-4(5H)- yl]carbonyl}phenyl) ethanesulfonamide |
| 168 | 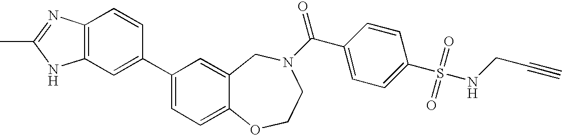 | 4-{[7-(2-methyl-1H- benzimidazol-6-yl)-2,3-dihydro- 1,4-benzoxazepin-4(5H)- yl]carbonyl}-N-prop-2-yn-1- ylbenzenesulfonamide |
| 169 | 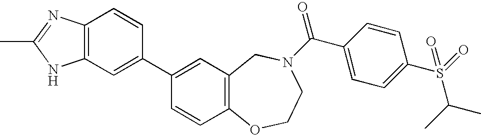 | 7-(2-methyl-1H-benzimidazol-6- yl)-4-({4-[(1- methylethyl)sulfonyl]phenyl} carbonyl)-2,3,4,5-tetrahydro-1,4- benzoxazepine |
| 170 | 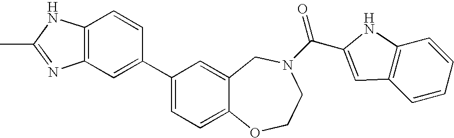 | 4-(1H-indol-2-ylcarbonyl)-7-(2- methyl-1H-benzimidazol-5-yl)- 2,3,4,5-tetrahydro-1,4- benzoxazepine |
| 171 | 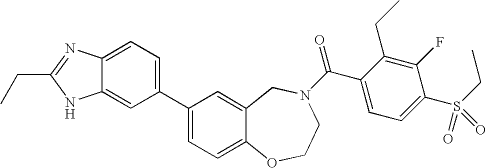 | 7-(2-ethyl-1H-benzimidazol-6- yl)-4-{[2-ethyl-4- (ethylsulfonyl)-3- fluorophenyl]carbonyl}-2,3,4,5- tetrahydro-1,4-benzoxazepine |
| 172 | 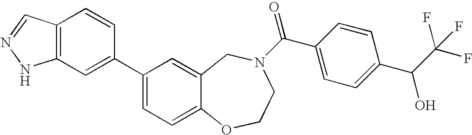 | 2,2,2-trifluoro-1-(4-{[7-(1H- indazol-6-yl)-2,3-dihydro-1,4- benzoxazepin-4(5H)- yl]carbonyl}phenyl)ethanol |
| 173 | 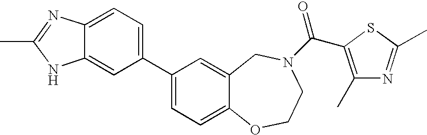 | 4-[(2,4-dimethyl-1,3-thiazol-5- yl)carbonyl]-7-(2-methyl-1H- benzimidazol-6-yl)-2,3,4,5- tetrahydro-1,4-benzoxazepine |
| 174 | 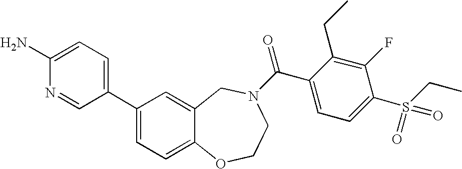 | 5-(4-{[2-ethyl-4-(ethylsulfonyl)- 3-fluorophenyl]carbonyl}- 2,3,4,5-tetrahydro-1,4- benzoxazepin-7-yl)pyridin-2- amine |
| 175 | 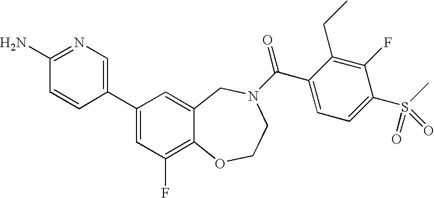 | 5-(4-{[2-ethyl-3-fluoro-4- (methylsulfonyl)phenyl]carbonyl}- 9-fluoro-2,3,4,5-tetrahydro- 1,4-benzoxazepin-7-yl)pyridin- 2-amine |
| 176 | 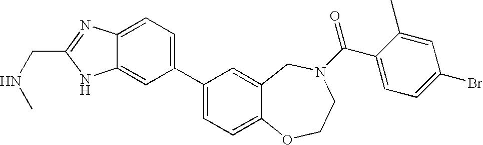 | l-(6-{4-[(4-bromo-2- methylphenyl)carbonyl]-2,3,4,5- tetrahydro-1,4-benzoxazepin-7- yl}-1H-benzimidazol-2-yl)-N- methylmethanamine |
| 177 | 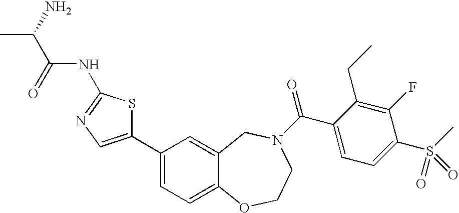 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]-L-alaninamide |
| 178 | 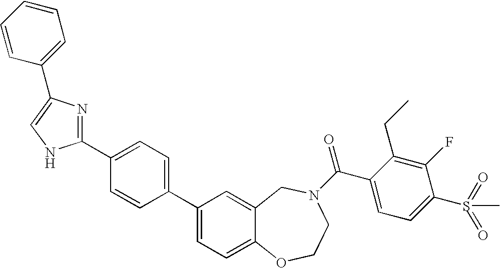 | 4-{[2-ethyl-3-fluoro-4- (methylsulfonyl)phenyl]carbonyl}- 7-[4-(4-phenyl-1H-imidazol-2- yl)phenyl]-2,3,4,5-tetrahydro- 1,4-benzoxazepine |
| 179 | 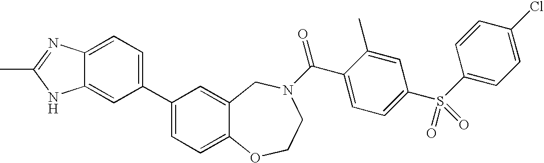 | 4-({4-[(4- chlorophenyl)sulfonyl]-2- methylphenyl}carbonyl)-7-(2- methyl-1H-benzimidazol-6-yl)- 2,3,4,5-tetrahydro-1,4- benzoxazepine |
| 180 | 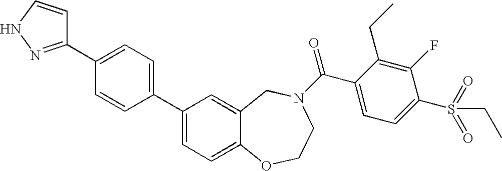 | 4-{[2-ethyl-4-(ethylsulfonyl)-3- fluorophenylcarbonyl}-7-[4- (1H-pyrazol-3-yl)phenyl]- 2,3,4,5-tetrahydro-1,4- benzoxazepine |
| 181 | 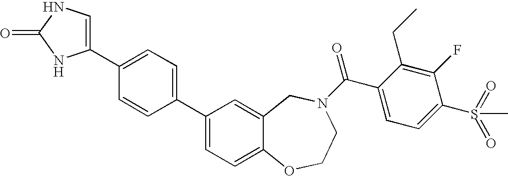 | 4-[4-(4-{[2-ethyl-3-fluoro-4- (methylsulfonyl)phenyl]carbonyl}- 2,3,4,5-tetrahydro-1,4- benzoxazepin-7-yl)phenyl]-1,3- dihydro-2H-imidazol-2-one |
| 182 | 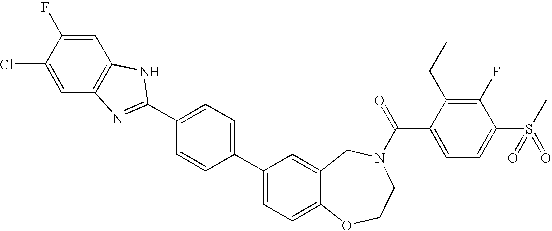 | 7-[4-(5-chloro-6-fluoro-1H- benzimidazol-2-y])phenyl]-4- {[2-ethyl-3-fluoro-4- (methylsulfonyl)phenyl]carbonyl}- 2,3,4,5-tetrahydro-1,4- benzoxazepine |
| 183 | 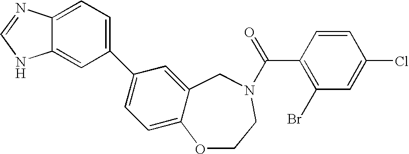 | 7-(1H-benzimidazol-6-yl)-4-[(2- bromo-4- chlorophenyl)carbonyl]-2,3,4,5- tetrahydro-1,4-benzoxazepine |
| 184 | 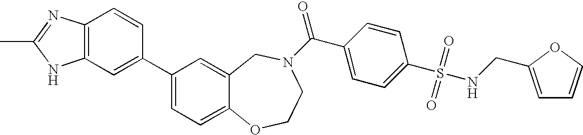 | N-(furan-2-ylmethyl)-4-{[7-(2- methyl-1H-benzimidazol-6-yl)- 2,3-dihydro-1,4-benzoxazepin- 4(5H)- yl]carbonyl}benzenesulfonamide |
| 185 | 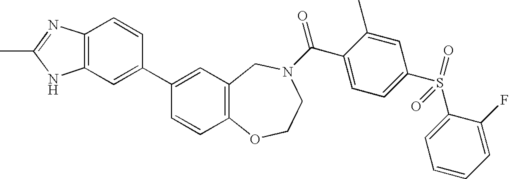 | 4-({4-[(2- fluoropheny])sulfonyl]-2- methylphenyl}carbonyl)-7-(2- methyl-1H-benzimidazol-6-yl)- 2,3,4,5-tetrahydro-1,4- benzoxazepine |
| 186 | 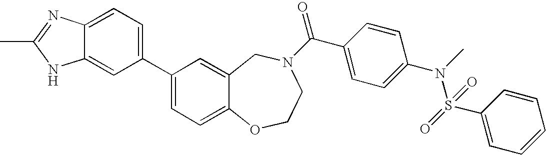 | N-methyl-N-(4-{[7-(2-methyl- 1H-benzimidazol-6-yl)-2,3- dihydro-1,4-benzoxazepin- 4(5H)-yl]carbonyl}phenyl) benzenesulfonamide |
| 187 | 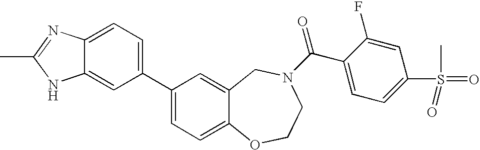 | 4-{[2-fluoro-4- (methylsulfonyl)phenyl]carbonyl}- 7-(2-methyl-1H-benzimidazol- 6-yl)-2,3,4,5-tetrahydro-1,4- benzoxazepine |
| 188 | 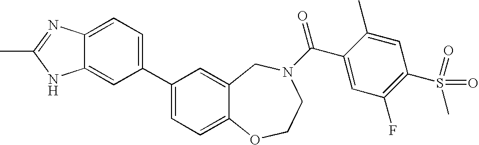 | 4-{[5-fluoro-2-methyl-4- (methylsulfonyl)phenyl]carbonyl}- 7-(2-methyl-1H-benzimidazol- 6-yl)-2,3,4,5-tetrahydro-1,4- benzoxazepine |
| 189 | 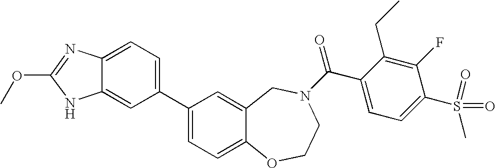 | 4-{[2-ethyl-3-fluoro-4- (methylsulfonyl)phenyl]carbonyl}- 7-[2-(methyloxy)-1H- benzimidazol-6-yl]-2,3,4,5- tetrahydro-1,4-benzoxazepine |
| 190 | 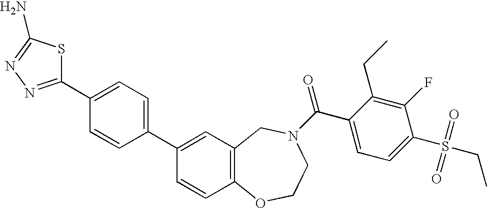 | 5-[4-(4-{[2-ethyl-4- (ethylsulfonyl)-3- fluorophenyl]carbonyl}-2,3,4,5- tetrahydro-1,4-benzoxazepin-7- yl)phenyl]-1,3,4-thiadiazol-2- amine |
| 191 | 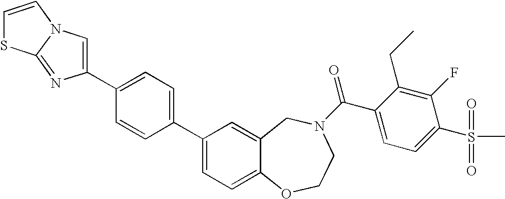 | 4-{[2-ethyl-3-fluoro-4- (methylsulfonyl)phenyl]carbonyl}- 7-(4-imidazo[2,1- b][1,3]thiazol-6-ylphenyl)- 2,3,4,5-tetrahydro-1,4- benzoxazepine |
| 192 | 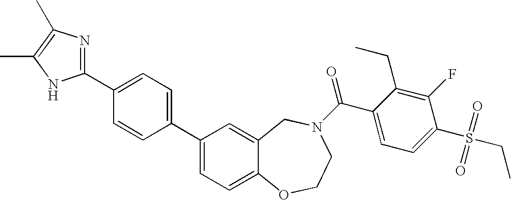 | 7-[4-(4,5-dimethyl-1H-imidazol- 2-yl)phenyl]-4-{[2-ethyl-4- (ethylsulfonyl)-3- fluorophenyl]carbonyl}-2,3,4,5- tetrahydro-1,4-benzoxazepine |
| 193 | 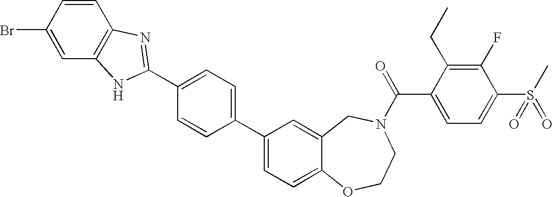 | 7-[4-(6-bromo-1H- benzimidazol-2-y])phenyl]-4- {[2-ethyl-3-fluoro-4- (methylsulfonyl)phenyl]carbonyl}- 2,3,4,5-tetrahydro-1,4- benzoxazepine |
| 194 | 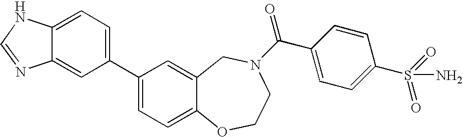 | 4-{[7-(1H-benzimidazol-5-yl)- 2,3-dihydro-1,4-benzoxazepin- 4(5H)- yl]carbonyl}benzenesulfonamide |
| 195 | 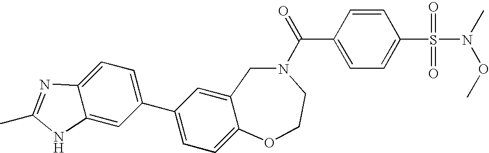 | N-methyl-4-{[7-(2-methyl-1H- benzimidazol-6-yl)-2,3-dihydro- 1,4-benzoxazepin-4(5H)- yl]carbonyl}-N- (methyloxy)benzenesulfonamide |
| 196 | 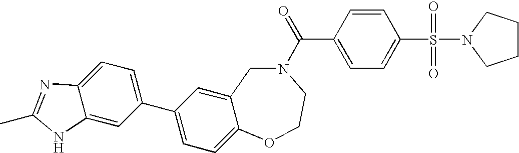 | 7-(2-methyl-1H-benzimidazol-6- yl)-4-{[4-(pyrrolidin-1- ylsulfonyl)phenyl]carbonyl}- 2,3,4,5-tetrahydro-1,4- benzoxazepine |
| 197 | 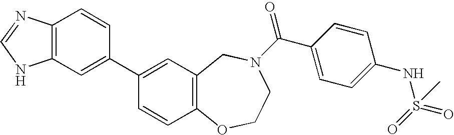 | N(4-{[7-(1H-benzimidazol-6- yl)-2,3-dihydro-1,4- benzoxazepin-4(5H)- yl]carbonyl}phenyl) methanesulfonamide |
| 198 | 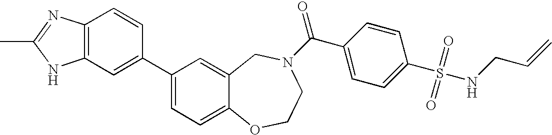 | 4-{[7-(2-methyl-1H- benzimidazol-6-yl)-2,3-dihydro- 1,4-benzoxazepin-4(5H)- yl]carbonyl}-N-prop-2-en-1- ylbenzenesulfonamide |
| 199 | 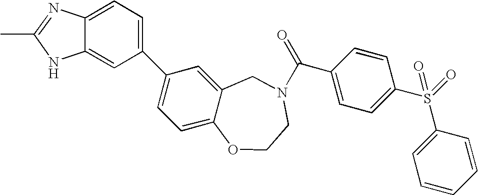 | 7-(2-methyl-1H-benzimidazol-6- yl)-4-{[4- (phenylsulfonyl)phenyl]carbonyl}- 2,3,4,5-tetrahydro-1,4- benzoxazepine |
| 200 | 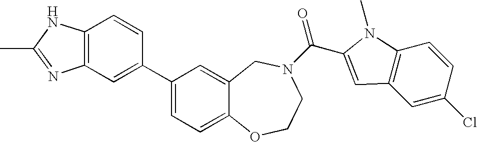 | 4-[(5-chloro-1-methyl-1H-indol- 2-yl)carbonyl]-7-(2-methyl-1H- benzimidazol-5-yl)-2,3,4,5- tetrahydro-1,4-benzoxazepine |
| 201 | 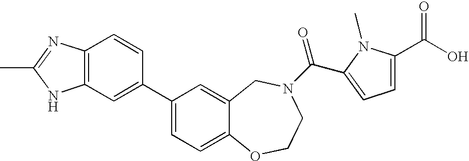 | 1-methyl-5-{[7-(2-methyl-1H- benzimidazol-6-yl)-2,3-dihydro- 1,4-benzoxazepin-4(5H)- yl]carbonyl}-1H-pyrrole-2- carboxylic acid |
| 202 | 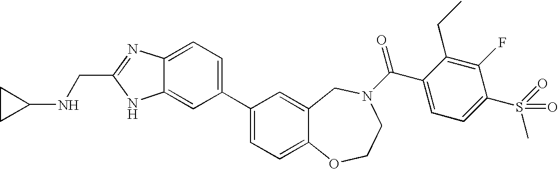 | N-{[6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2- yl]methyl}cyclopropanamine |
| 203 | 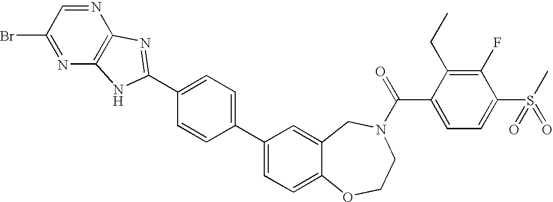 | 7-[4-(6-bromo-1H-imidazo[4,5- b]pyrazin-2-yl)phenyl]-4-{[2- ethyl-3-fluoro-4- (methylsulfonyl)phenyl]carbonyl}- 2,3,4,5-tetrahydro-1,4- benzoxazepine |
| 204 | 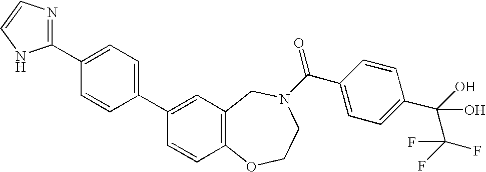 | 2,2,2-trifluoro-1-[4-({7-[4-(1H- imidazol-2-yl)phenyl]-2,3- dihydro-1,4-benzoxazepin- 4(5H)- yl}carbonyl)phenyl]ethane-1,1- diol |
| 205 | 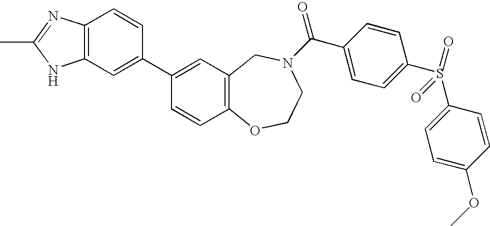 | 7-(2-methyl-1H-benzimidazol-6- yl)-4-[(4-{[4- (methyloxy)phenyl]sulfonyl}phenyl) carbonyl]-2,3,4,5-tetrahydro- 1,4-benzoxazepine |
| 206 | 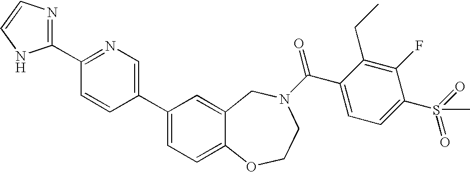 | 4-{[2-ethyl-3-fluoro-4- (methylsulfonyl)phenyl]carbonyl}- 7-[6-(1H-imidazol-2- yl)pyridin-3-yl]-2,3,4,5- tetrahydro-1,4-benzoxazepine |
| 207 | 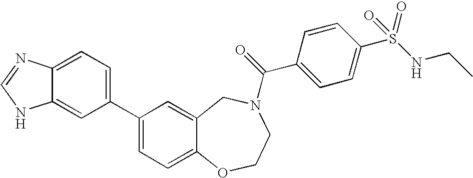 | 4-{[7-(lH-benzimidazol-6-yl)- 2,3-dihydro-1,4-benzoxazepin- 4(5H)-yl]carbonyl}-N- ethylbenzenesulfonamide |
| 208 | 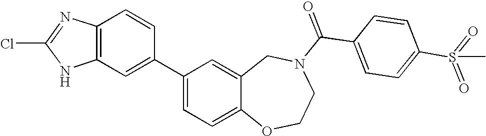 | 7-(2-chloro-1H-benzimidazol-6- yl)-4-{[4- (methylsulfonyl)phenyl]carbonyl}- 2,3,4,5-tetrahydro-1,4- benzoxazepine |
| 209 | 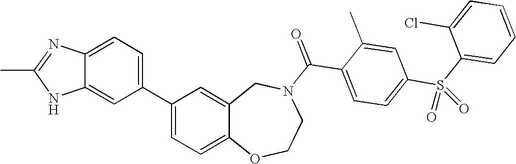 | 4-({4-[(2- chlorophenyl)sulfonyl]-2- methylphenyl}carbonyl)-7-(2- methyl-1H-benzimidazol-6-yl)- 2,3,4,5-tetrahydro-1,4- benzoxazepine |
| 210 | 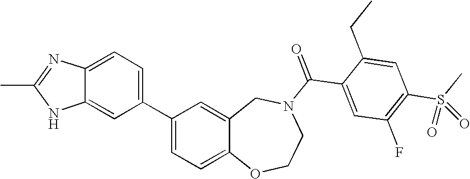 | 4-{[2-ethyl-5-fluoro-4- (methylsulfonyl)phenyl]carbonyl}- 7-(2-methyl-1H-benzimidazol- 6-yl)-2,3,4,5-tetrahydro-1,4- benzoxazepine |
| 211 | 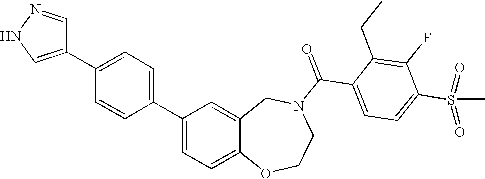 | 4-{[2-ethyl-3-fluoro-4- (methylsulfonyl)phenyl]carbonyl}- 7-[4-(1H-pyrazol-4- yl)phenyl]-2,3,4,5-tetrahydro- 1,4-benzoxazepine |
| 212 | 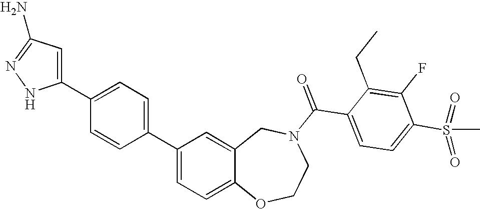 | 5-[4-(4-{[2-ethyl-3-fluoro-4- (methylsulfonyl)phenyl]carbonyl}- 2,3,4,5-tetrahydro-1,4- benzoxazepin-7-yl)phenyl]-1H- pyrazol-3-amine |
| 213 | 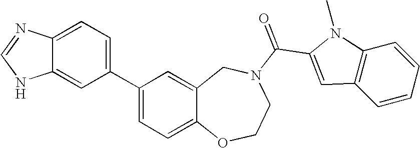 | 7-(1H-benzimidazol-6-yl)-4-[(1- methyl-1H-indol-2-yl)carbonyl]- 2,3,4,5-tetrahydro-1,4- benzoxazepine |
| 214 | 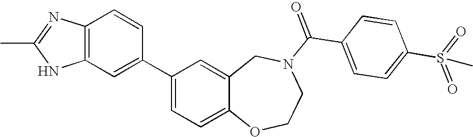 | 7-(2-methyl-1H-benzimidazol-6- yl)-4-{[4- (methylsulfonyl)phenyl]carbonyl}- 2,3,4,5-tetrahydro-1,4- benzoxazepine |
| 215 | 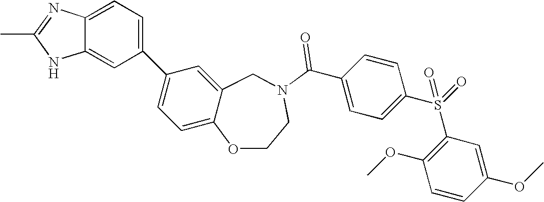 | 4-[(4-{[2,5- bis(methyloxy)phenyl]sulfonyl} phenyl)carbonyl]-7-(2-methyl- 1H-benzimidazol-6-yl)-2,3,4,5- tetrahydro-1,4-benzoxazepine |
| 216 | 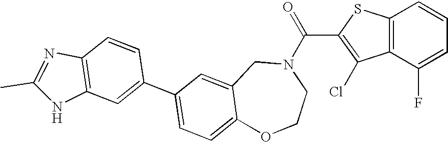 | 4-[(3-chloro-4-fluoro-1- benzothien-2-yl)carbonyl]-7-(2- methyl-1H-benzimidazol-6-yl)- 2,3,4,5-tetrahydro-1,4- benzoxazepine |
| 217 | 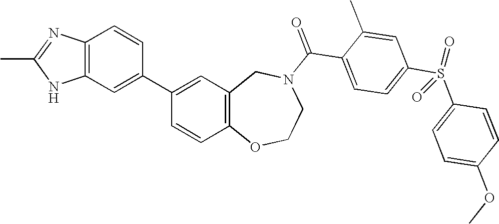 | 7-(2-methyl-1H-benzimidazol-6- yl)-4-[(2-methyl-4-{[4- (methyloxy)phenyl]sulfonyl} phenyl)carbonyl]-2,3,4,5- tetrahydro-1,4-benzoxazepine |
| 218 | 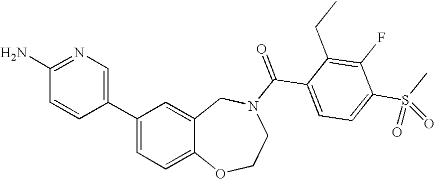 | 5-(4-{[2-ethyl-3-fluoro-4- (methylsulfonyl)phenyl]carbonyl}- 2,3,4,5-tetrahydro-1,4- benzoxazepin-7-yl)pyridin-2- amine |
| 219 | 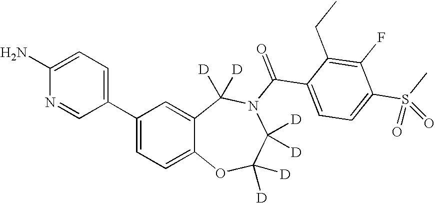 | [7-(6-aminopyridin-3- yl)(2,2,3,3,5,5-²H₆)-2,3-dihydro- 1,4-benzoxazepin-4(5H)-yl][2- ethyl-3-fluoro-4- (methylsulfonyl)phenyl]methanone |
| 220 | 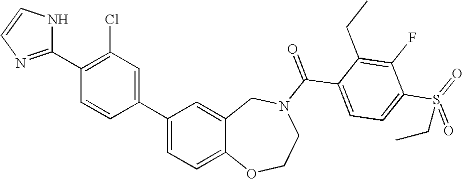 | 7-[3-chloro-4-(1H-imidazol-2- yl)phenyl]-4-{[2-ethyl-4- (ethylsulfonyl)-3- fluorophenyl]carbonyl}-2,3,4,5- tetrahydro-1,4-benzoxazepine |
| 221 | 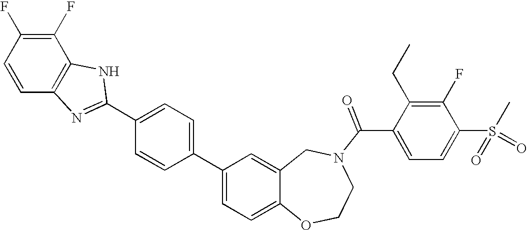 | 7-[4-(6,7-difluoro-1H- benzimidazol-2-yl)phenyl]-4- {[2-ethyl-3-fluoro-4- (methylsulfonyl)phenyl]carbonyl}- 2,3,4,5-tetrahydro-1,4- benzoxazepine |
| 222 | 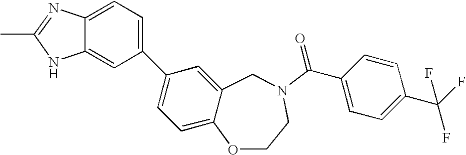 | 7-(2-methyl-1H-benzimidazol-6- yl)-4-{[4- (trifluoromethyl)phenyl]carbonyl}- 2,3,4,5-tetrahydro-1,4- benzoxazepine |
| 223 | 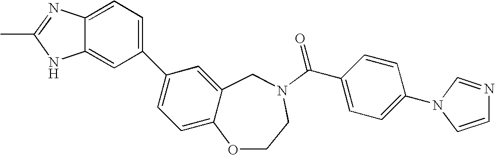 | 4-{[4-(1H-imidazol-1- yl)phenyl]carbonyl}-7-(2- methyl-1H-benzimidazol-5-yl)- 2,3,4,5-tetrahydro-1,4- benzoxazepine |
| 224 | 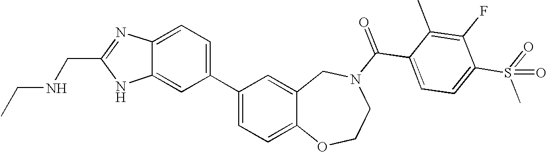 | N-{[6-(4-{[3-fluoro-2-methyl-4- (methylsulfonyl)phenyl]carbonyl}- 2,3,4,5-tetrahydro-1,4- benzoxazepin-7-yl)-1H- benzimidazol-2- yl]methyl}ethanamine |
| 225 | 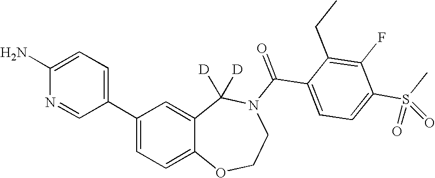 | [7-(6-aminopyridin-3-yl)(5,5- ²H₂)-2,3-dihydro-1,4- benzoxazepin-4(5H)-yl][2-ethyl- 3-fluoro-4- (methylsulfonyl)phenyl]methanone |
| 226 | 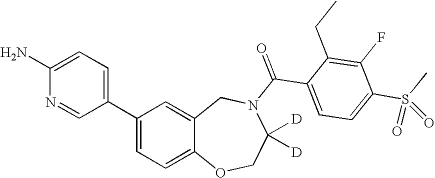 | [7-(6-aminopyridin-3-yl)(3,3- ²H₂)-2,3-dihydro-1,4- benzoxazepin-4(5H)-yl][2-ethyl- 3-fluoro-4- (methylsulfonyl)phenyl]methanone |
| 227 | 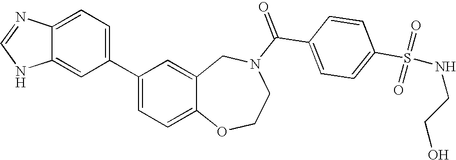 | 4-{[7-(1H-benzimidazol-6-yl)- 2,3-dihydro-1,4-benzoxazepin- 4(5H)-yl]carbonyl}-N-(2- hydroxyethyl)benzenesulfonamide |
| 228 | 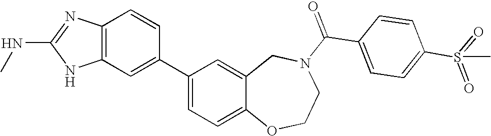 | N-methyl-6-(4-{[4- (methylsulfonyl)phenyl]carbonyl}- 2,3,4,5-tetrahydro-1,4- benzoxazepin-7-yl)-1H- benzimidazol-2-amine |
| 229 | 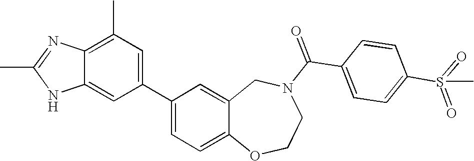 | 7-(2,4-dimethyl-1H- benzimidazol-6-yl)-4-{[4- (methylsulfonyl)phenyl]carbonyl}- 2,3,4,5-tetrahydro-1,4- benzoxazepine |
| 230 | 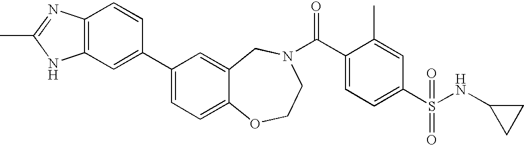 | N-cyclopropyl-3-methyl-4-{[7- (2-methyl-1H-benzimidazol-6- yl)-2,3-dihydro-1,4- benzoxazepin-4(5H)- yl]carbonyl}benzenesulfonamide |
| 231 | 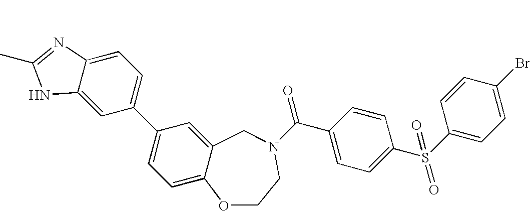 | 4-({4-[(4- bromophenyl)sulfonyl]phenyl} carbonyl)-7-(2-methyl-1H- benzimidazol-6-yl)-2,3,4,5- tetrahydro-1,4-benzoxazepine |
| 232 | 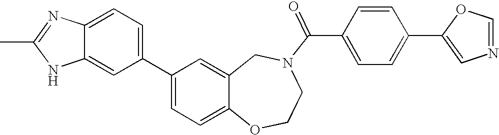 | 7-(2-methyl-1H-benzimidazol-6- yl)-4-{[4-(1,3-oxazol-5- yl)phenyl]carbonyl}-2,3,4,5- tetrahydro-1,4-benzoxazepine |
| 233 | 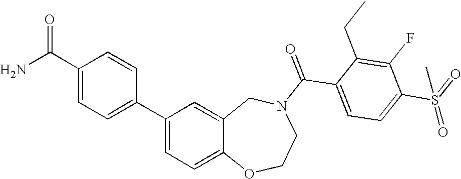 | 4-(4-{[2-ethyl-3-fluoro-4- (methylsulfonyl)phenyl]carbonyl}- 2,3,4,5-tetrahydro-1,4- benzoxazepin-7-yl)benzamide |
| 234 | 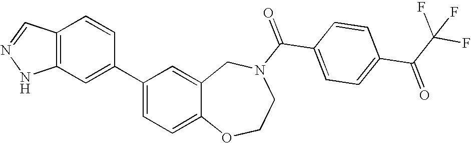 | 2,2,2-trifluoro-1-(4-{[7-(1H- indazol-6-yl)-2,3-dihydro-1,4- benzoxazepin-4(5H)- yl]carbonyl}phenyl)ethanone |
| 235 | 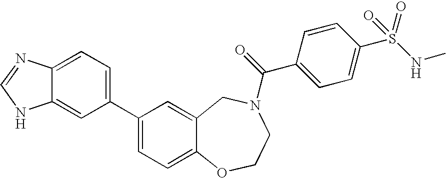 | 4-{[7-(1H-benzimidazol-6-yl)- 2,3-dihydro-1,4-benzoxazepin- 4(5H)-yl]carbonyl}-N- methylbenzenesulfonamide |
| 236 | 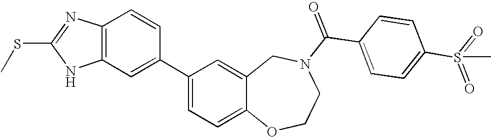 | 4-{[4- (methylsulfonyl)phenyl]carbonyl}- 7-[2-(methylthio)-1H- benzimidazol-6-yl]-2,3,4,5- tetrahydro-1,4-benzoxazepine |
| 237 | 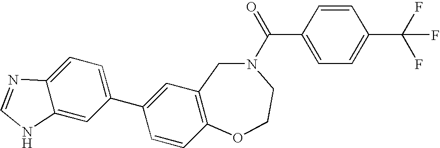 | 7-(1H-benzimidazol-6-yl)-4-{[4- (trifluoromethyl)phenyl]carbonyl}- 2,3,4,5-tetrahydro-1,4- benzoxazepine |
| 238 | 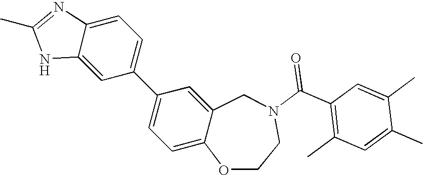 | 7-(2-methyl-1H-benzimidazol-6- yl)-4-[(2,4,5- trimethylphenyl)carbonyl]- 2,3,4,5-tetrahydro-1,4- benzoxazepine |
| 239 | 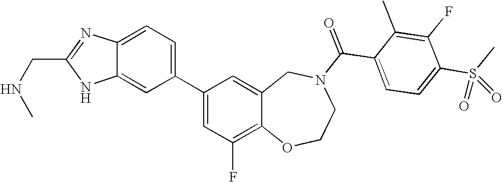 | l-[6-(9-fluoro-4-{[3-fluoro-2- methyl-4- (methylsulfonyl)phenyl]carbonyl}- 2,3,4,5-tetrahydro-1,4- benzoxazepin-7-yl)-1H- benzimidazol-2-yl]-N- methylmethanamine |
| 240 | 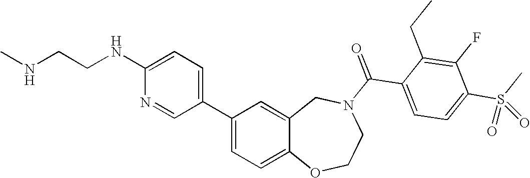 | N-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- N′-methylethane-1,2-diamine |
| 241 | 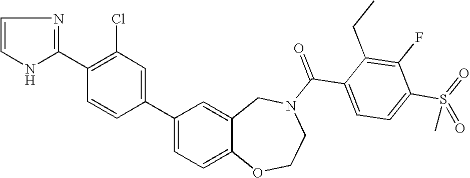 | 7-[3-chloro-4-(1H-imidazol-2- yl)phenyl]-4-{[2-ethyl-3-fluoro-4- (methylsulfonyl)phenyl]carbonyl}- 2,3,4,5-tetrahydro-1,4- benzoxazepine |
| 242 | 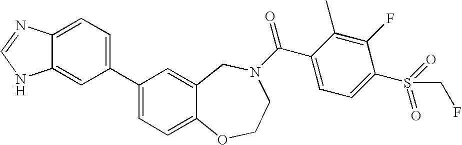 | 7-(1H-benzimidazol-6-yl)-4-({3- fluoro-4- [(fluoromethyl)sulfonyl]-2- methylphenyl}carbonyl)-2,3,4,5- tetrahydro-1,4-benzoxazepine |
| 243 | 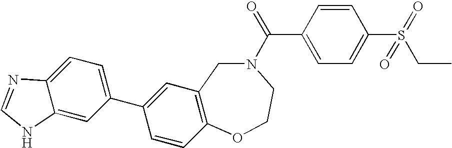 | 7-(1H-benzimidazol-6-yl)-4-{[4- (ethylsulfonyl)phenyl]carbonyl}- 2,3,4,5-tetrahydro-1,4- benzoxazepine |
| 244 | 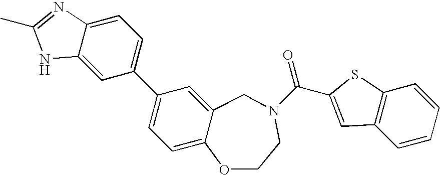 | 4-(1-benzothien-2-ylcarbony])- 7-(2-methyl-1H-benzimidazol-6- yl)-2,3,4,5-tetrahydro-1,4- benzoxazepine |
| 245 | 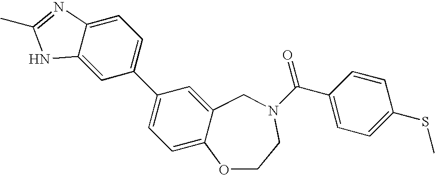 | 7-(2-methyl-1H-benzimidazol-5- yl)-4-{[4- (methylthio)phenyl]carbonyl}- 2,3,4,5-tetrahydro-1,4- benzoxazepine |
| 246 | 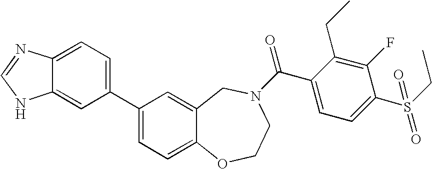 | 7-(1H-benzimidazol-6-yl)-4-{[2- ethyl-4-(ethylsulfonyl)-3- fluorophenyl]carbonyl}-2,3,4,5- tetrahydro-1,4-benzoxazepine |
| 247 | 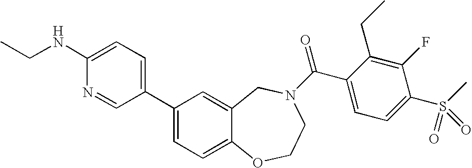 | N-ethyl-5-(4-{[2-ethyl-3-fluoro-4- (methylsulfonyl)phenyl]carbonyl}- 2,3,4,5-tetrahydro-1,4- benzoxazepin-7-yl)pyridin-2- amine |
| 248 | 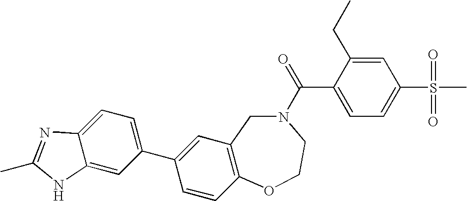 | 4-{[2-ethyl-4- (methylsulfonyl)phenyl]carbonyl}- 7-(2-methyl-1H-benzimidazol- 6-yl)-2,3,4,5-tetrahydro-1,4- benzoxazepine |
| 249 | 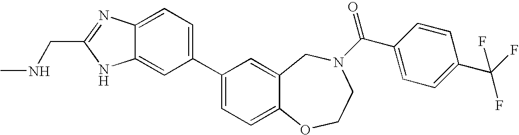 | N-methyl-l-[6-(4-{[4- (trifluoromethyl)phenyl]carbonyl}- 2,3,4,5-tetrahydro-1,4- benzoxazepin-7-yl)-1H- benzimidazol-2-yl]methanamine |
| 250 | 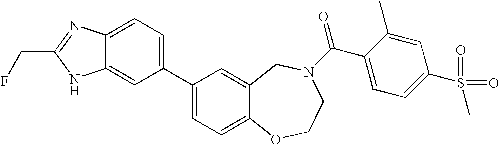 | 7-[2-(fluoromethyl)-1H- benzimidazol-6-yl]-4-{[2- methyl-4- (methylsulfonyl)phenyl]carbonyl}- 2,3,4,5-tetrahydro-1,4- benzoxazepine |
| 251 | 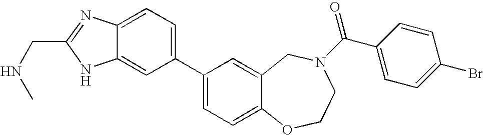 | 1-(6-{4-[(4- bromophenyl)carbonyl]-2,3,4,5- tetrahydro-1,4-benzoxazepin-7- yl}-1H-benzimidazol-2-yl)-N- methylmethanamine |
| 252 | 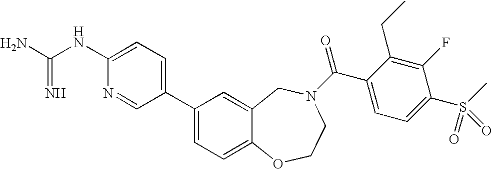 | 1-[5-(4-{[2-ethyl-3-fluoro-4- (methylsulfonyl)phenyl]carbonyl}- 2,3,4,5-tetrahydro-1,4- benzoxazepin-7-yl)pyridin-2- yl]guanidine |
| 253 | 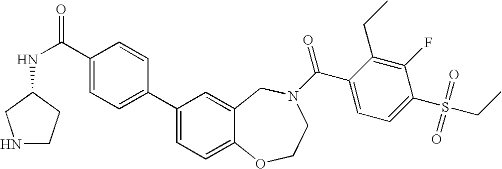 | 4-(4-{[2-ethyl-4-(ethylsulfonyl)- 3-fluorophenyl]carbonyl}- 2,3,4,5-tetrahydro-1,4- benzoxazepin-7-yl)-N-[(3R)- pyrrolidin-3-yl]benzamide |
| 254 | 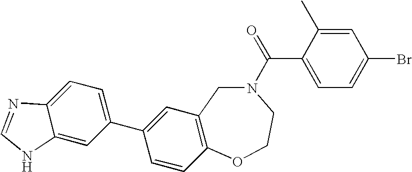 | 7-(1H-benzimidazol-6-yl)-4-[(4- bromo-2- methylphenyl)carbonyl]-2,3,4,5- tetrahydro-1,4-benzoxazepine |
| 255 | 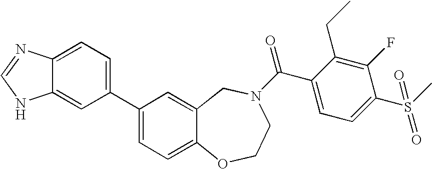 | 7-(1H-benzimidazol-6-yl)-4-{[2- ethyl-3-fluoro-4- (methylsulfonyl)phenyl]carbonyl}- 2,3,4,5-tetrahydro-1,4- benzoxazepine |
| 256 | 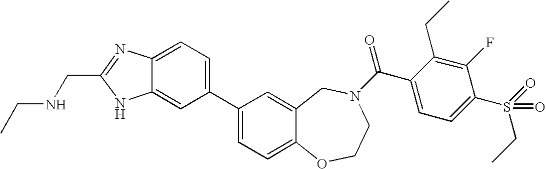 | N-{[6-(4-{[2-ethyl-4- (ethylsulfonyl)-3- fluorophenyl]carbonyl}-2,3,4,5- tetrahydro-1,4-benzoxazepin-7- yl)-1H-benzimidazol-2- yl]methyl}ethanamine |
| 257 | 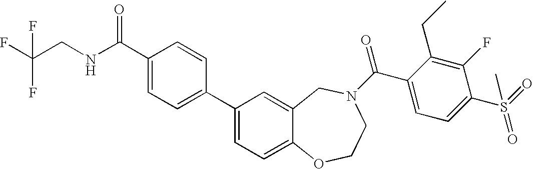 | 4-(4-{[2-ethyl-3-fluoro-4- (methylsulfonyl)phenyl]carbonyl}- 2,3,4,5-tetrahydro-1,4- benzoxazepin-7-yl)-N-(2,2,2- trifluoroethyl)benzamide |
| 258 | 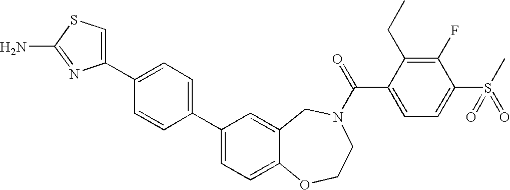 | 4-[4-(4-{[2-ethyl-3-fluoro-4- (methylsulfonyl)phenyl]carbonyl}- 2,3,4,5-tetrahydro-1,4- benzoxazepin-7-yl)phenyl]-1,3- thiazol-2-amine |
| 259 | 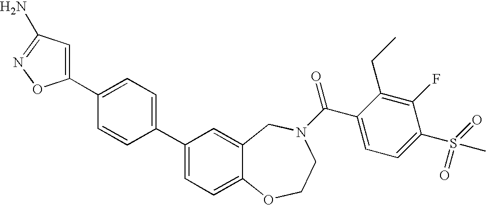 | 5-[4-(4-{[2-ethyl-3-fluoro-4- (methylsulfonyl)phenyl]carbonyl}- 2,3,4,5-tetrahydro-1,4- benzoxazepin-7- yl)phenyl]isoxazol-3-amine |
| 260 | 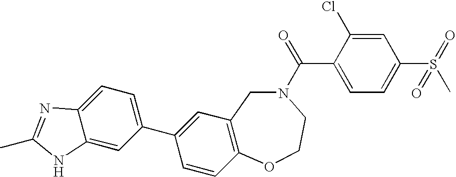 | 4-{[2-chloro-4- (methylsulfonyl)phenyl]carbonyl}- 7-(2-methyl-1H-benzimidazol- 6-yl)-2,3,4,5-tetrahydro-1,4- benzoxazepine |
| 261 | 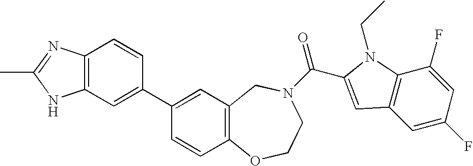 | 4-[(1-ethyl-5,7-difluoro-1H- indol-2-yl)carbonyl]-7-(2- methyl-1H-benzimidazol-6-yl)- 2,3,4,5-tetrahydro-1,4- benzoxazepine |
| 262 | 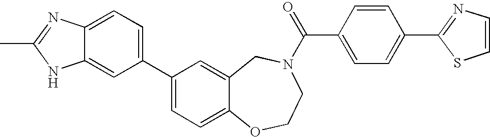 | 7-(2-methyl-1H-benzimidazol-6- yl)-4-{[4-(1,3-thiazol-2- yl)phenyl]carbonyl}-2,3,4,5- tetrahydro-1,4-benzoxazepine |
| 263 | 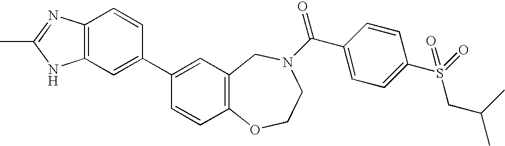 | 7-(2-methyl-1H-benzimidazol-6- yl)-4-({4-[(2- methylpropyl)sulfonyl]phenyl} carbonyl)-2,3,4,5-tetrahydro-1,4- benzoxazepine |
| 264 | 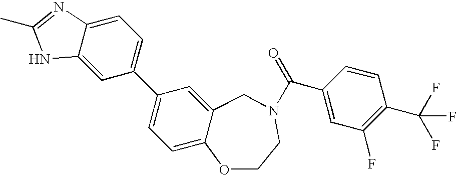 | 4-{[3-fluoro-4- (trifluoromethyl)phenyl]carbonyl}- 7-(2-methyl-1H- benzimidazol-5-yl)-2,3,4,5- tetrahydro-1,4-benzoxazepine |
| 265 | 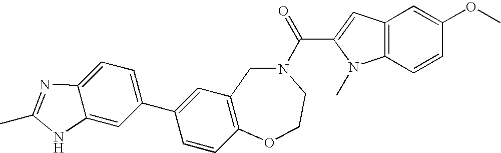 | 7-(2-methyl-1H-benzimidazol-6- yl)-4-{[1-methyl-5-(methyloxy)- 1H-indol-2-yl]carbonyl}-2,3,4,5- tetrahydro-1,4-benzoxazepine |
| 266 | 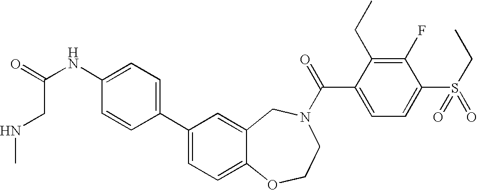 | N-[4-(4-{[2-ethyl-4- (ethylsulfonyl)-3- fluoropheny]carbonyl}-2,3,4,5- tetrahydro-1,4-benzoxazepin-7- yl)phenyl]-N²- methylglycinamide |
| 267 | 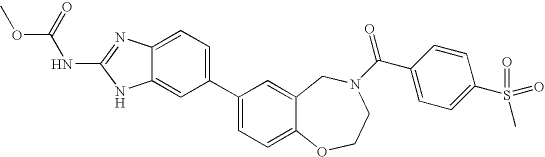 | methyl [6-(4-{[4- (methylsulfonyl)phenyl]carbonyl}- 2,3,4,5-tetrahydro-1,4- benzoxazepin-7-yl)-1H- benzimidazol-2-yl]carbamate |
| 268 | 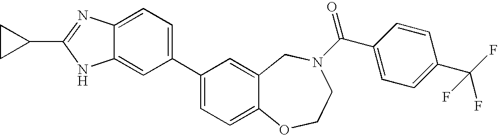 | 7-(2-cyclopropyl-1H- benzimidazol-6-yl)-4-{[4- (trifluoromethyl)phenyl]carbonyl}- 2,3,4,5-tetrahydro-1,4- benzoxazepine |
| 269 | 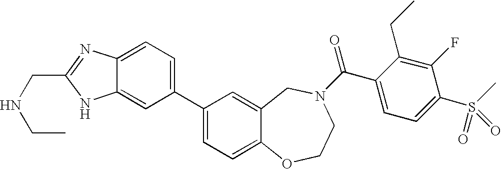 | N-{[6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2- yl]methyl}ethanamine |
| 270 | 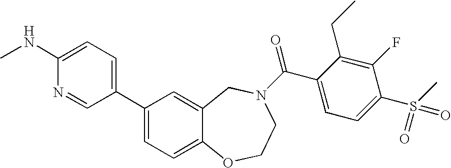 | 5-(4-{[2-ethyl-3-fluoro-4- (methylsulfonyl)phenyl]carbonyl}- 2,3,4,5-tetrahydro-1,4- benzoxazepin-7-yl)-N- methylpyridin-2-amine |
| 271 | 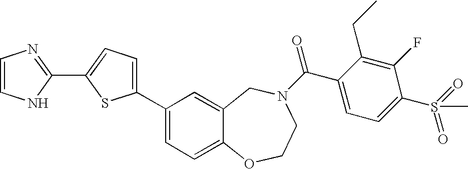 | 4-{[2-ethyl-3-fluoro-4- (methylsulfonyl)phenyl]carbonyl}- 7-[5-(1H-imidazol-2-yl)-2- thienyl]-2,3,4,5-tetrahydro-1,4- benzoxazepine |
| 272 | 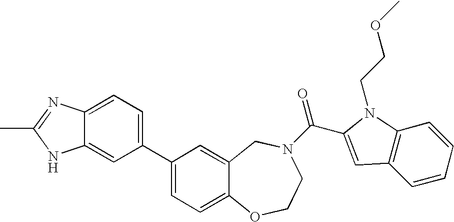 | 7-(2-methyl-1H-benzimidazol-6- yl)-4-({1-[2-(methyloxy)ethyl]- 1H-indol-2-yl}carbonyl)-2,3,4,5- tetrahydro-1,4-benzoxazepine |
| 273 | 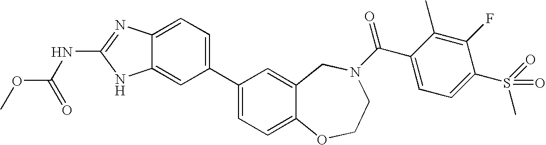 | methyl [6-(4-{[3-fluoro-2- methyl-4- (methylsulfonyl)phenyl]carbonyl}- 2,3,4,5-tetrahydro-1,4- benzoxazepin-7-yl)-1H- benzimidazol-2-yl]carbamate |
| 274 | 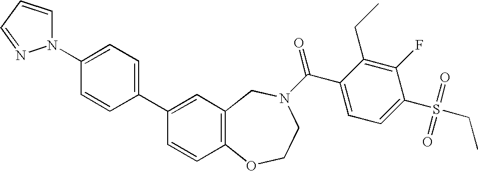 | 4-{[2-ethyl-4-(ethylsulfonyl)-3- fluorophenyl]carbonyl}-7-[4- (1H-pyrazol-1-yl)phenyl]- 2,3,4,5-tetrahydro-1,4- benzoxazepine |
| 275 | 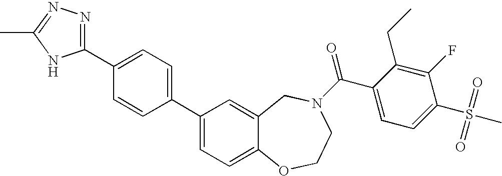 | 4-{[2-ethyl-3-fluoro-4- (methylsulfonyl)phenyl]carbonyl}- 7-[4-(5-methyl-4H-1,2,4- triazol-3-yl)phenyl]-2,3,4,5- tetrahydro-1,4-benzoxazepine |
| 276 | 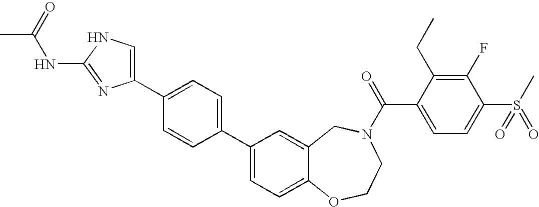 | N-{4-[4-(4-{[2-ethyl-3-fluoro-4- (methylsulfonyl)phenyl]carbonyl}- 2,3,4,5-tetrahydro-1,4- benzoxazepin-7-yl)phenyl]-1H- imidazol-2-yl}acetamide |
| 277 | 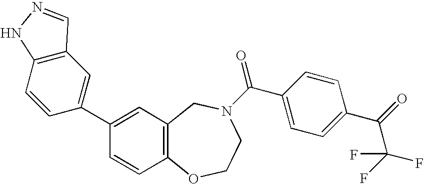 | 2,2,2-trifluoro-1-(4-{[7-(1H- indazol-5-yl)-2,3-dihydro-1,4- benzoxazepin-4(5H)- yl]carbonyl}phenyl)ethanone |
| 278 | 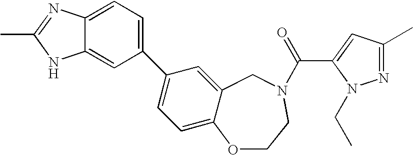 | 4-[(1-ethyl-3-methyl-1H- pyrazol-5-yl)carbonyl]-7-(2- methyl-1H-benzimidazol-6-yl)- 2,3,4,5-tetrahydro-1,4- benzoxazepine |
| 279 | 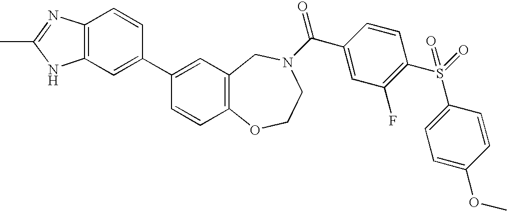 | 4-[(3-fluoro-4-{[4- (methyloxy)phenyl]sulfonyl}phenyl) carbonyl]-7-(2-methyl-1H- benzimidazol-6-yl)-2,3,4,5- tetrahydro-1,4-benzoxazepine |
| 280 | 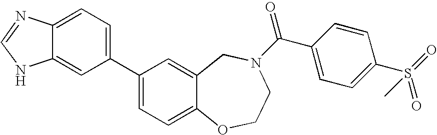 | 7-(1H-benzimidazol-6-yl)-4-{[4- (methylsulfonyl)phenyl]carbonyl}- 2,3,4,5-tetrahydro-1,4- benzoxazepine |
| 281 | 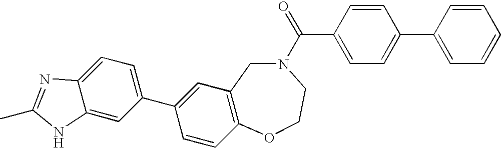 | 4-(biphenyl-4-ylcarbonyl)-7-(2- methyl-1H-benzimidazol-6-yl)- 2,3,4,5-tetrahydro-1,4- benzoxazepine |
| 282 | 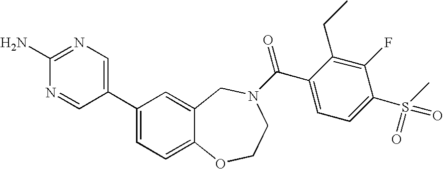 | 5-(4-{[2-ethyl-3-fluoro-4- (methylsulfonyl)phenyl]carbonyl}- 2,3,4,5-tetrahydro-1,4- benzoxazepin-7-yl)pyrimidin-2- amine |
| 283 | 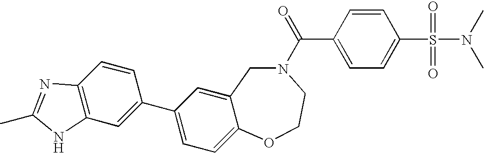 | N,N-dimethyl-4-{[7-(2-methyl- 1H-benzimidazol-6-yl)-2,3- dihydro-1,4-benzoxazepin- 4(5H)- yl]carbonyl}benzenesulfonamide |
| 284 | 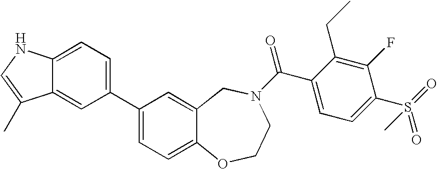 | 4-{[2-ethyl-3-fluoro-4- (methylsulfonyl)phenyl]carbonyl}- 7-(3-methyl-1H-indol-5-yl)- 2,3,4,5-tetrahydro-1,4- benzoxazepine |
| 285 | 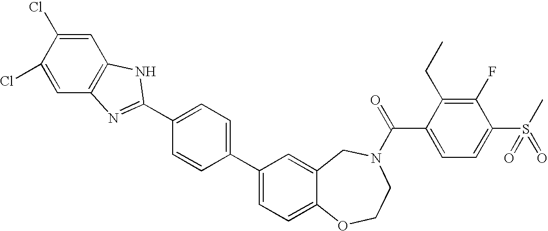 | 7-[4-(5,6-dichloro-1H- benzimidazol-2-yl)phenyl]-4- {[2-ethyl-3-fluoro-4- (methylsulfonyl)phenyl]carbonyl}- 2,3,4,5-tetrahydro-1,4- benzoxazepine |
| 286 | 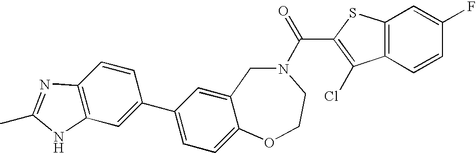 | 4-[(3-chloro-6-fluoro-1- benzothien-2-yl)carbonyl]-7-(2- methyl-1H-benzimidazol-6-yl)- 2,3,4,5-tetrahydro-1,4- benzoxazepine |
| 287 | 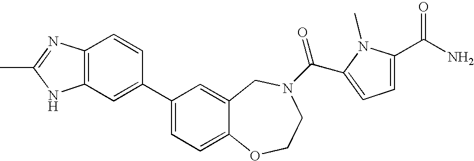 | 1-methyl-5-{[7-(2-methyl-1H- benzimidazol-6-yl)-2,3-dihydro- 1,4-benzoxazepin-4(5H)- yl]carbonyl}-1H-pyrrole-2- carboxamide |
| 288 | 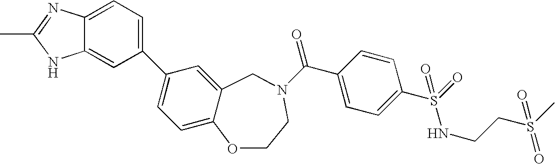 | 4-{[7-(2-methyl-1H- benzimidazol-6-yl)-2,3-dihydro- 1,4-benzoxazepin-4(5H)- yl]carbonyl}-N-[2- (methylsulfonyl)ethyl] benzenesulfonamide |
| 289 | 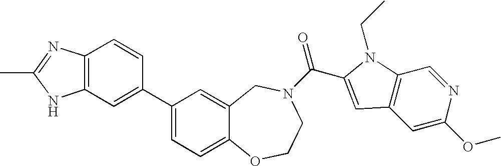 | 4-{[1-ethyl-5-(methyloxy)-1H- pyrrolo[2,3-c]pyridin-2- yl]carbonyl}-7-(2-methyl-1H- benzimidazol-6-yl)-2,3,4,5- tetrahydro-1,4-benzoxazepine |
| 290 | 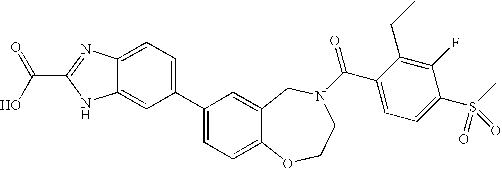 | 6-(4-{[2-ethyl-3-fluoro-4- (methylsulfonyl)phenyl]carbonyl}- 2,3,4,5-tetrahydro-1,4- benzoxazepin-7-yl)-1H- benzimidazole-2-carboxylic acid |
| 291 | 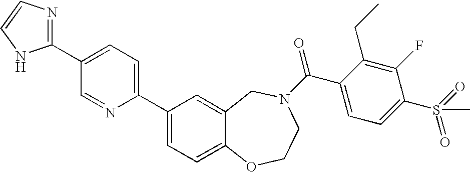 | 4-{[2-ethyl-3-fluoro-4- (methylsulfonyl)phenyl]carbonyl}- 7-[5-(1H-imidazol-2- yl)pyridin-2-yl]-2,3,4,5- tetrahydro-1,4-benzoxazepine |
| 292 | 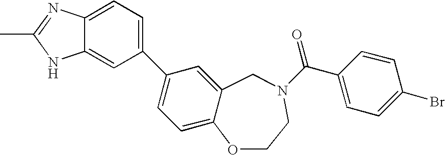 | 4-[(4-bromophenyl)carbonyl]-7- (2-methyl-1H-benzimidazol-6- yl)-2,3,4,5-tetrahydro-1,4- benzoxazepine |
| 293 | 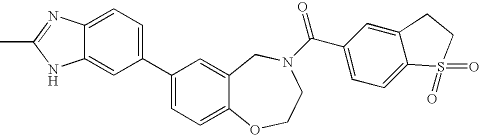 | 4-[(1,1-dioxido-2,3-dihydro-1- benzothien-5-yl)carbonyl]-7-(2- methyl-1H-benzimidazol-6-yl)- 2,3,4,5-tetrahydro-1,4- benzoxazepine |
| 294 | 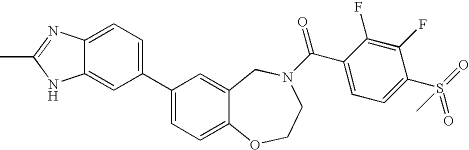 | 4-{[2,3-difluoro-4- (methylsulfonyl)phenyl]carbonyl}- 7-(2-methyl-1H-benzimidazol- 6-yl)-2,3,4,5-tetrahydro-1,4- benzoxazepine |
| 295 | 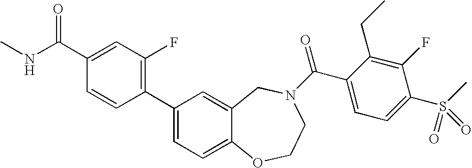 | 4-(4-{[2-ethyl-3-fluoro-4- (methylsulfonyl)phenyl]carbonyl}- 2,3,4,5-tetrahydro-1,4- benzoxazepin-7-yl)-3-fluoro-N- methylbenzamide |
| 296 | 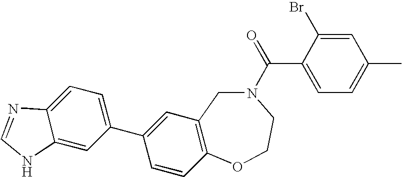 | 7-(1H-benzimidazol-6-yl)-4-[(2- bromo-4- methylphenyl)carbonyl]-2,3,4,5- tetrahydro-1,4-benzoxazepine |
| 297 | 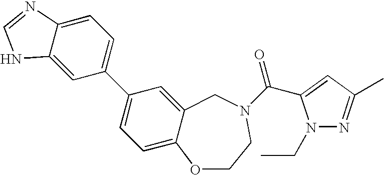 | 7-(1H-benzimidazol-6-yl)-4-[(1- ethyl-3-methyl-1H-pyrazol-5- yl)carbonyl]-2,3,4,5-tetrahydro- 1,4-benzoxazepine |
| 298 | 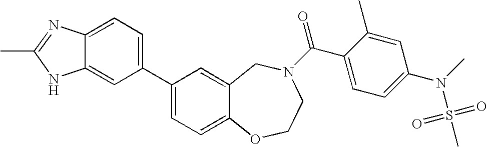 | N-methyl-N-(3-methyl-4-{[7-(2- methyl-1H-benzimidazol-6-yl)- 2,3-dihydro-1,4-benzoxazepin- 4(5H)-yl]carbonyl}phenyl) methanesulfonamide |
| 299 | 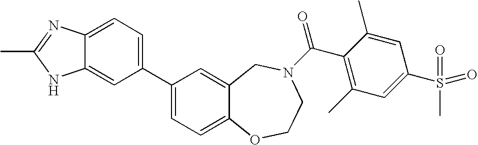 | 4-{[2,6-dimethyl-4- (methylsulfonyl)phenyl]carbonyl}- 7-(2-methyl-1H-benzimidazol- 6-yl)-2,3,4,5-tetrahydro-1,4- benzoxazepine |
| 300 | 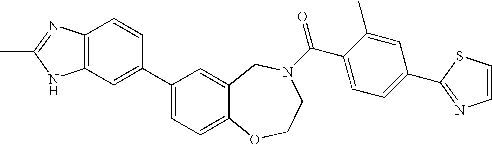 | 7-(2-methyl-1H-benzimidazol-6- yl)-4-{[2-methyl-4-(1,3-thiazol- 2-yl)phenyl]carbonyl}-2,3,4,5- tetrahydro-1,4-benzoxazepine |
| 301 | 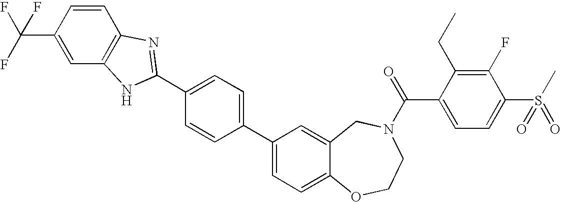 | 4-{[2-ethyl-3-fluoro-4- (methylsulfonyl)phenyl]carbonyl}- 7-{4-[6-(trifluoromethyl)-1H- benzimidazol-2-yl]phenyl}- 2,3,4,5-tetrahydro-1,4- benzoxazepine |
| 302 | 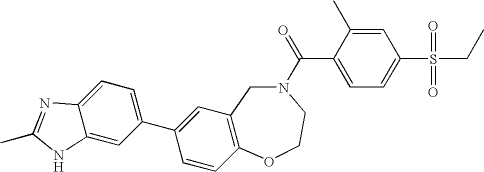 | 4-{[4-(ethylsulfonyl)-2- methylphenyl]carbonyl}-7-(2- methyl-1H-benzimidazol-6-yl)- 2,3,4,5-tetrahydro-1,4- benzoxazepine |
| 303 | 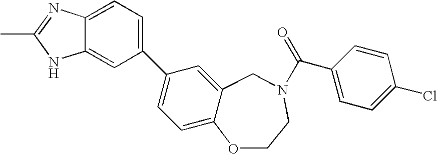 | 4-[(4-chlorophenyl)carbonyl]-7- (2-methyl-1H-benzimidazol-6- yl)-2,3,4,5-tetrahydro-1,4- benzoxazepine |
| 304 | 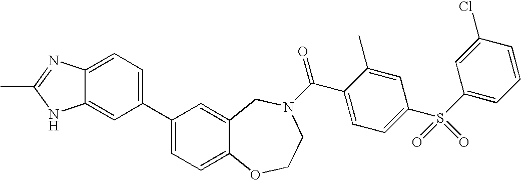 | 4-({4-[(3- chlorophenyl)sulfonyl]-2- methylphenyl}carbonyl)-7-(2- methyl-1H-benzimidazol-6-yl)- 2,3,4,5-tetrahydro-1,4- benzoxazepine |
| 305 | 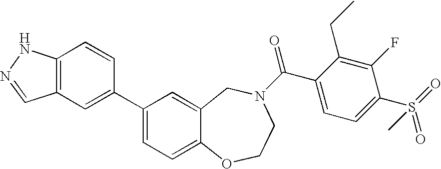 | 4-{[2-ethyl-3-fluoro-4- (methylsulfonyl)phenyl]carbonyl}- 7-(1H-indazol-5-yl)-2,3,4,5- tetrahydro-1,4-benzoxazepine |
| 306 | 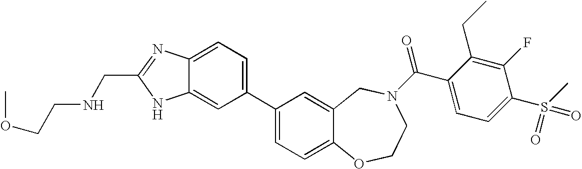 | N-{[6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2-yl]methyl}-2- (methyloxy)ethanamine |
| 307 | 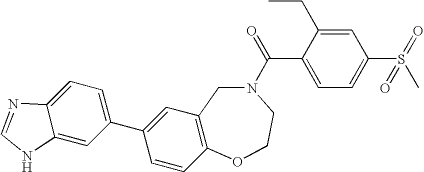 | 7-(1H-benzimidazol-6-yl)-4-{[2- ethyl-4- (methylsulfonyl)phenyl]carbonyl}- 2,3,4,5-tetrahydro-1,4- benzoxazepine |
| 308 | 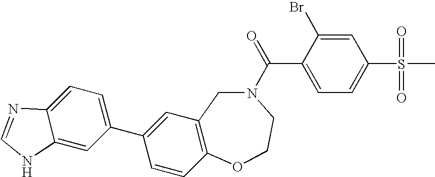 | 7-(1H-benzimidazol-6-yl)-4-{[2- bromo-4- (methylsulfonyl)phenyl]carbonyl}- 2,3,4,5-tetrahydro-1,4- benzoxazepine |
| 309 | 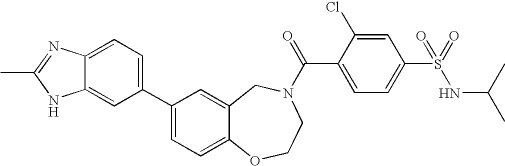 | 3-chloro-4-{[7-(2-methyl-1H- benzimidazol-6-yl)-2,3-dihydro- 1,4-benzoxazepin-4(5H)- yl]carbonyl}-N-(1- methylethyl)benzenesulfonamide |
| 310 | 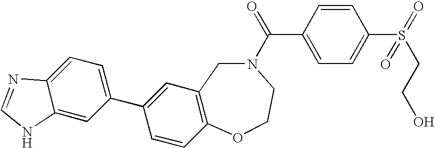 | 2-[(4-{[7-(1H-benzimidazol-6- yl)-2,3-dihydro-1,4- benzoxazepin-4(5H)- yl]carbonyl}phenyl)sulfonyl] ethanol |
| 311 | 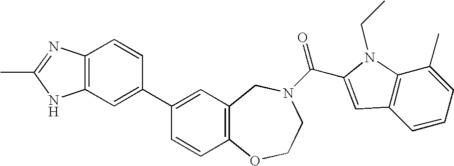 | 4-[(1-ethyl-7-methyl-1H-indol- 2-yl)carbonyl]-7-(2-methyl-1H- benzimidazol-6-yl)-2,3,4,5- tetrahydro-1,4-benzoxazepine |
| 312 | 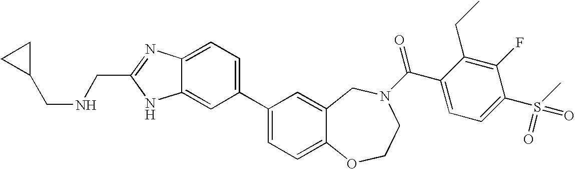 | 1-cyclopropyl-N-{[6-(4-{[2- ethyl-3-fluoro-4- (methylsulfonyl)phenyl]carbonyl}- 2,3,4,5-tetrahydro-1,4- benzoxazepin-7-yl)-1H- benzimidazol-2- yl]methyl}methanamine |
| 313 | 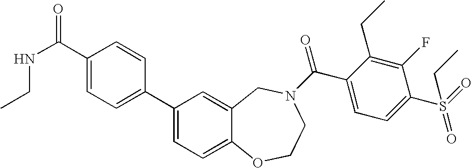 | N-ethyl-4-(4-{[2-ethyl-4- (ethylsulfonyl)-3- fluorophenyl]carbonyl}-2,3,4,5- tetrahydro-1,4-benzoxazepin-7- yl)benzamide |
| 314 | 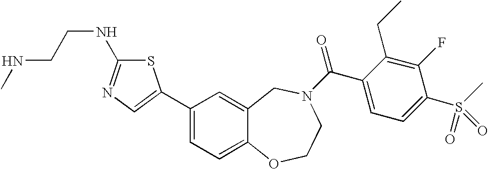 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]-N′-methylethane-1,2- diamine |
| 315 | 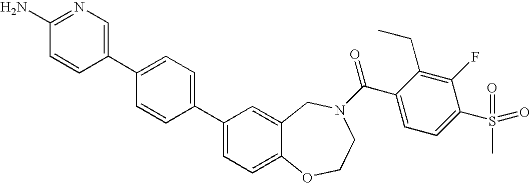 | 5-[4-(4-{[2-ethyl-3-fluoro-4- (methylsulfonyl)phenyl]carbonyl}- 2,3,4,5-tetrahydro-1,4- benzoxazepin-7- yl)phenyl]pyridin-2-amine |
| 316 | 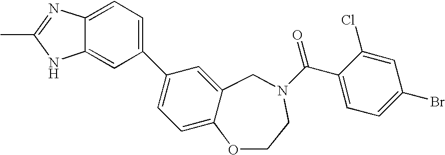 | 4-[(4-bromo-2- chlorophenyl)carbonyl]-7-(2- methyl-1H-benzimidazol-6-yl)- 2,3,4,5-tetrahydro-1,4- benzoxazepine |
| 317 | 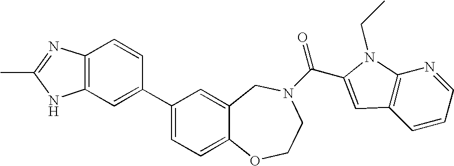 | 4-[(1-ethyl-1H-pyrrolo[2,3- b]pyridin-2-yl)carbonyl]-7-(2- methyl-1H-benzimidazol-6-yl)- 2,3,4,5-tetrahydro-1,4- benzoxazepine |
| 318 | 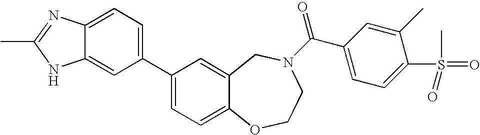 | 7-(2-methyl-1H-benzimidazol-6- yl)-4-{[3-methyl-4- (methylsulfonyl)phenyl]carbonyl}- 2,3,4,5-tetrahydro-1,4- benzoxazepine |
| 319 | 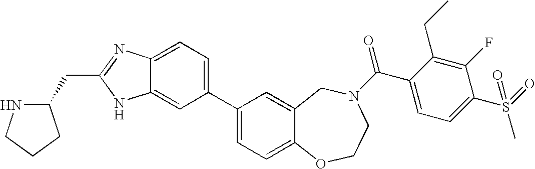 | 4-{[2-ethyl-3-fluoro-4- (methylsulfonyl)phenyl]carbonyl}- 7-{2-[(2S)-pyrrolidin-2- ylmethyl]-1H-benzimidazol-6- yl}-2,3,4,5-tetrahydro-1,4- benzoxazepine |
| 320 | 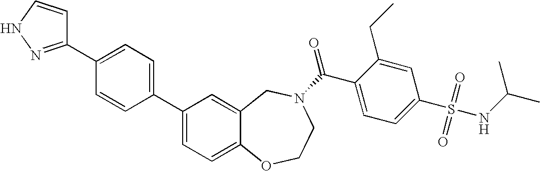 | 3-ethy-N-(1-methylethyl)-4-({7- [4-(1H-pyrazol-3-yl)phenyl]-2,3- dihydro-1,4-benzoxazepin- 4(5H)- yl}carbonyl)benzenesulfonamide |
| 321 | 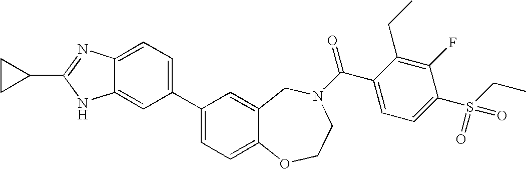 | 7-(2-cyclopropyl-1H- benzimidazol-6-yl)-4-{[2-ethyl- 4-(ethylsulfonyl)-3- fluorophenyl]carbonyl}-2,3,4,5- tetrahydro-1,4-benzoxazepine |
| 322 | 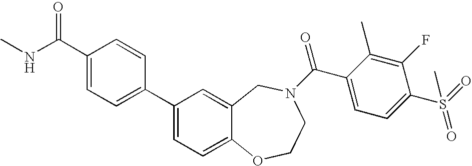 | 4-(4-{[3-fluoro-2-methyl-4- (methylsulfonyl)phenyl]carbonyl}- 2,3,4,5-tetrahydro-1,4- benzoxazepin-7-yl)-N- methylbenzamide |
| 323 | 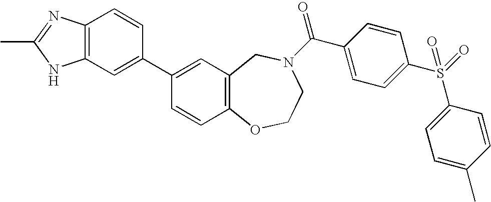 | 7-(2-methyl-1H-benzimidazol-6- yl)-4-({4-[(4- methylphenyl)sulfonyl]phenyl} carbony])-2,3,4,5-tetrahydro-1,4- benzoxazepine |
| 324 | 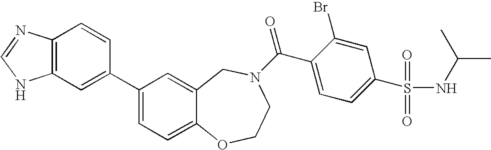 | 4-{[7-(1H-benzimidazol-6-yl)- 2,3-dihydro-1,4-benzoxazepin- 4(5H)-yl]carbonyl}-3-bromo-N-(1- methylethyl)benzenesulfonamide |
| 325 | 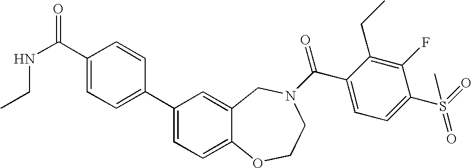 | N-ethyl-4-(4-{[2-ethyl-3-fluoro-4 (methylsulfonyl)phenyl]carbonyl- 2,3,4,5-tetrahydro-1,4- benzoxazepin-7-yl)benzamide |
| 326 | 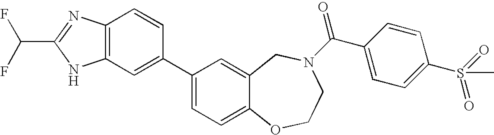 | 7-[2-(difluoromethyl)-1H- benzimidazol-6-yl]-4-{[4- (methylsulfonyl)phenyl]carbonyl}- 2,3,4,5-tetrahydro-1,4- benzoxazepine |
| 327 | 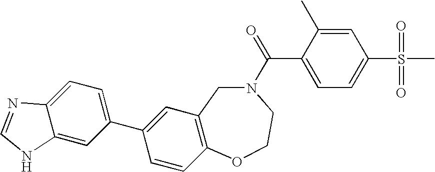 | 7-(1H-benzimidazol-6-yl)-4-{[2- methyl-4- (methylsulfonyl)phenyl]carbonyl}- 2,3,4,5-tetrahydro-1,4- benzoxazepine |
| 328 | 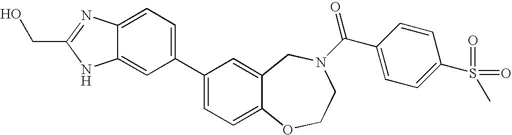 | [6-(4-{[4- (methylsulfonyl)phenyl]carbonyl}- 2,3,4,5-tetrahydro-1,4- benzoxazepin-7-yl)-1H- benzimidazol-2-yl]methanol |
| 329 | 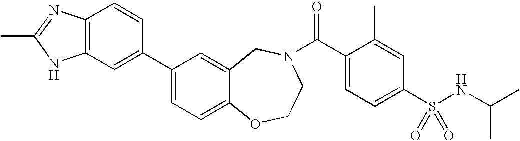 | 3-methyl-4-{[7-(2-methyl-1H- benzimidazol-6-yl)-2,3-dihydro- 1,4-benzoxazepin-4(5H)- yl]carbonyl}-N-(1- methylethyl)benzenesulfonamide |
| 330 | 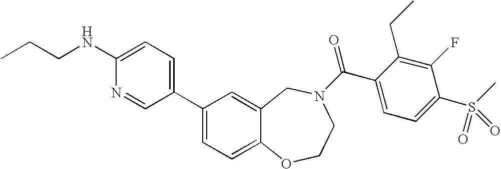 | 5-(4-{[2-ethyl-3-fluoro-4- (methylsulfonyl)phenyl]carbonyl}- 2,3,4,5-tetrahydro-1,4- benzoxazepin-7-yl)-N- propylpyridin-2-amine |
| 331 | 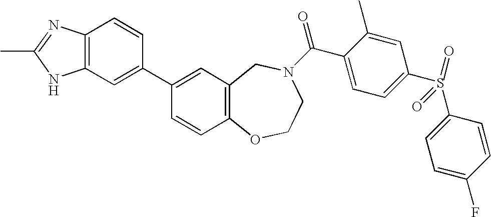 | 4-({4-[(4- fluoropheny])sulfonyl]-2- methylphenyl}carbonyl)-7-(2- methyl-1H-benzimidazol-6-yl)- 2,3,4,5-tetrahydro-1,4- benzoxazepine |
| 332 | 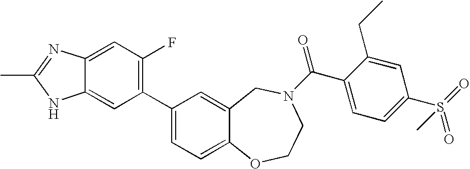 | 4-{[2-ethyl-4- (methylsulfonyl)phenyl]carbonyl}- 7-(5-fluoro-2-methyl-1H- benzimidazol-6-yl)-2,3,4,5- tetrahydro-1,4-benzoxazepine |
| 333 | 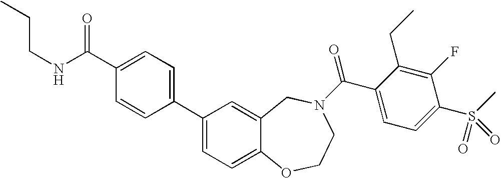 | 4-(4-{[2-ethyl-3-fluoro-4- (methylsulfonyl)phenyl]carbonyl}- 2,3,4,5-tetrahydro-1,4- benzoxazepin-7-yl)-N- propylbenzamide |
| 334 | 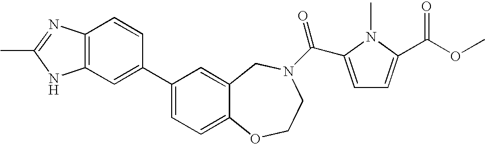 | methyl 1-methyl-5-{[7-(2- mcthyl-1H-benzimidazol-6-yl)- 2,3-dihydro-1,4-benzoxazepin- 4(5H)-yl]carbonyl}-1H-pyrrole- 2-carboxylate |
| 335 | 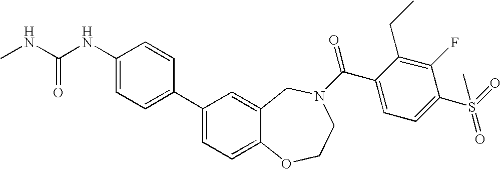 | l-[4-(4-{[2-ethyl-3-fluoro-4- (methylsulfonyl)phenyl]carbonyl}- 2,3,4,5-tetrahydro-1,4- benzoxazepin-7-yl)phenyl]-3- methylurea |
| 336 | 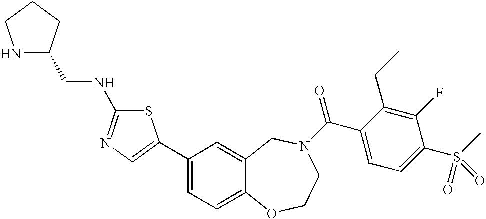 | 5-(4-{[2-ethyl-3-fluoro-4- (methylsulfonyl)phenyl]carbonyl}- 2,3,4,5-tetrahydro-1,4- benzoxazepin-7-yl)-N-[(2R)- pyrrolidin-2-ylmethyl]-1,3- thiazol-2-amine |
| 337 | 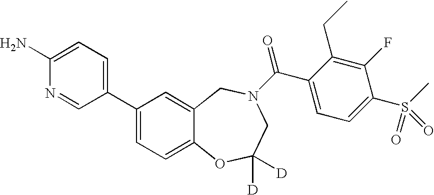 | [7-(6-aminopyridin-3-yl)(2,2- ²H₂)-2,3-dihydro-1,4- benzoxazepin-4(5H)-yl][2-ethyl- 3-fluoro-4- (methylsulfonyl)phenyl]methanone |
| 338 | 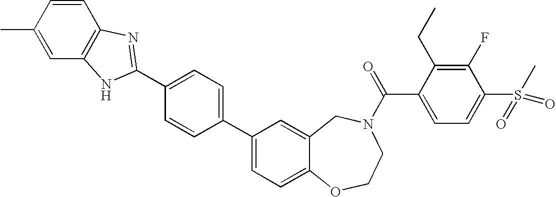 | 4-{[2-ethyl-3-fluoro-4- (methylsulfonyl)phenyl]carbonyl}- 7-[4-(6-methyl-1H- benzimidazol-2-yl)phenyl]- 2,3,4,5-tetrahydro-1,4- benzoxazepine |
| 339 | 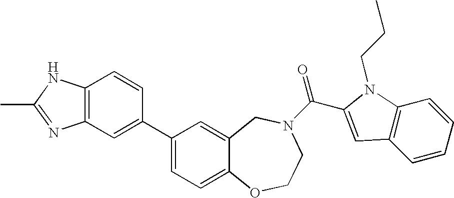 | 7-(2-methyl-1H-benzimidazol-5- yl)-4-[(1-propyl-1H-indol-2- yl)carbonyl]-2,3,4,5-tetrahydro- 1,4-benzoxazepine |
| 340 | 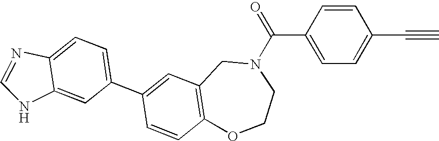 | 7-(1H-benzimidazol-6-yl)-4-[(4- ethynylphenyl)carbonyl]-2,3,4,5- tetrahydro-1,4-benzoxazepine |
| 341 | 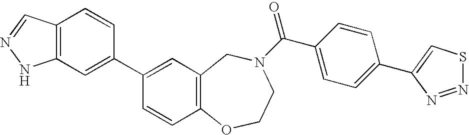 | 7-(1H-indazol-6-yl)-4-{[4- (1,2,3-thiadiazol-4- yl)phenyl]carbonyl}-2,3,4,5- tetrahydro-1,4-benzoxazepine |
| 342 | 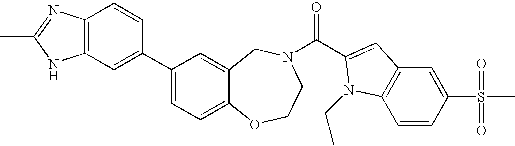 | 4-{[1-ethyl-5-(methylsulfonyl)- 1H-indol-2-yl]carbonyl}-7-(2- methyl-1H-benzimidazol-6-yl)- 2,3,4,5-tetrahydro-1,4- benzoxazepine |
| 343 | 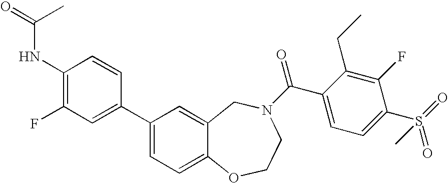 | N-[4-(4-{[2-ethyl-3-fluoro-4- (methylsulfonyl)phenyl]carbonyl}- 2,3,4,5-tetrahydro-1,4- benzoxazepin-7-yl)-2- fluorophenyl]acetamide |
| 344 | 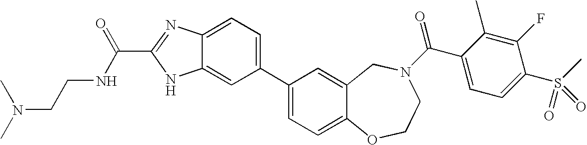 | N-[2-(dimethylamino)ethyl]-6- (4-{[3-fluoro-2-methyl-4- (methylsulfonyl)phenyl]carbonyl}- 2,3,4,5-tetrahydro-1,4- benzoxazepin-7-yl)-1H- benzimidazole-2-carboxamide |
| 345 | 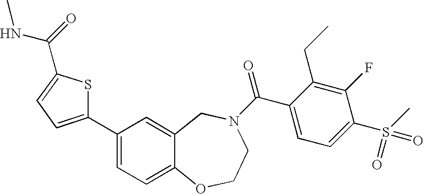 | 5-(4-{[2-ethyl-3-fluoro-4- (methylsulfonyl)phenyl]carbonyl}- 2,3,4,5-tetrahydro-1,4- benzoxazepin-7-yl)-N- methylthiophene-2-carboxamide |
| 346 | 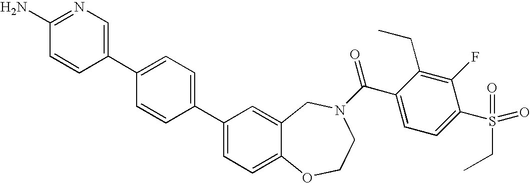 | 5-[4-(4-{[2-ethyl-4- (ethylsulfonyl)-3- fluorophenyl]carbonyl}-2,3,4,5- tetrahydro-1,4-benzoxazepin-7- yl)phenyl]pyridin-2-amine |
| 347 | 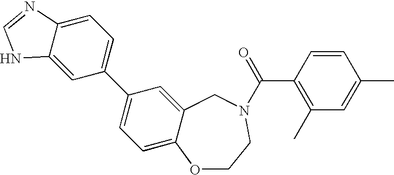 | 7-(1H-benzimidazol-6-yl)-4- [(2,4-dimethylphenyl)carbonyl]- 2,3,4,5-tetrahydro-1,4- benzoxazepine |
| 348 | 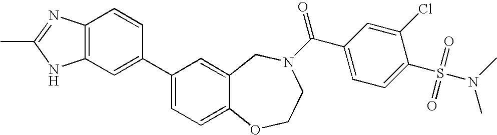 | 2-chloro-N,N-dimethyl-4-{[7-(2- methyl-1H-benzimidazol-6-yl)- 2,3-dihydro-1,4-benzoxazepin- 4(5H- yl]carbonyl}benzenesulfonamide |
| 349 | 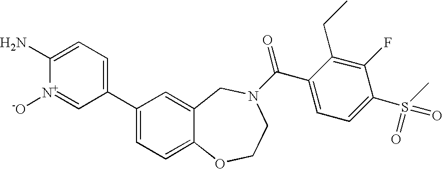 | 5-(4-{[2-ethyl-3-fluoro-4- (methylsulfonyl)phenyl]carbonyl}- 2,3,4,5-tetrahydro-1,4- benzoxazepin-7-yl)pyridin-2- amine 1-oxide |
| 350 | 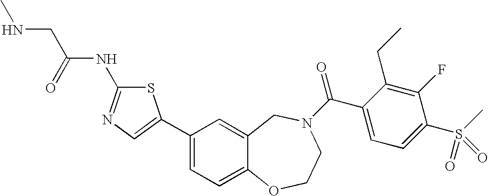 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]-N²-methylglycinamide |
| 351 | 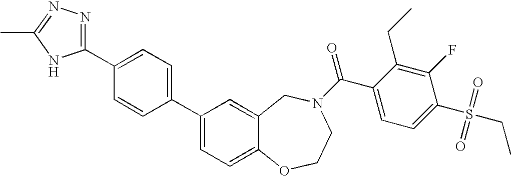 | 4-{[2-ethyl-4-(ethylsulfonyl)-3- fluorophenyl]carbonyl}-7-[4-(5- methy]-4H-1,2,4-triazol-3- yl)phenyl]-2,3,4,5-tetrahydro- 1,4-benzoxazepine |
| 352 | 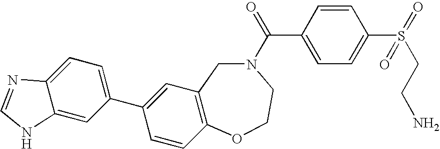 | 2-[(4-{[7-(1H-benzimidazol-6- yl)-2,3-dihydro-1,4- benzoxazepin-4(5H)- yl]carbonyl}phenyl)sulfonyl] ethanamine |
| 353 | 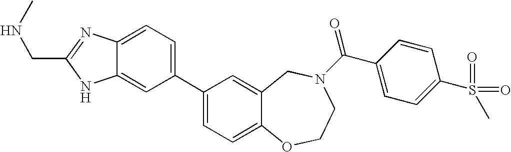 | N-methyl-1-[6-(4-{[4- (methylsulfonyl)phenyl]carbonyl}- 2,3,4,5-tetrahydro-1,4- benzoxazepin-7-yl)-1H- benzimidazol-2-yl]methanamine |
| 354 | 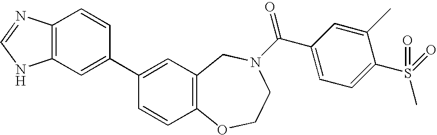 | 7-(1H-benzimidazol-6-yl)-4-{[3- methy]-4- (methylsulfonyl)phenyl]carbonyl}- 2,3,4,5-tetrahydro-1,4- benzoxazepine |
| 355 | 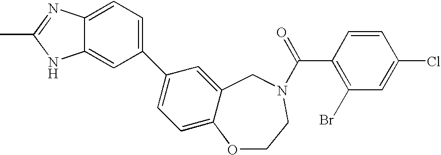 | 4-[(2-bromo-4- chlorophenyl)carbonyl]-7-(2- methyl-1H-benzimidazol-6-yl)- 2,3,4,5-tetrahydro-1,4- benzoxazepine |
| 356 | 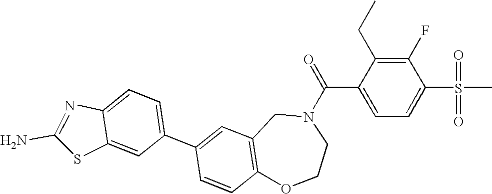 | 6-(4-{[2-ethyl-3-fluoro-4- (methylsulfonyl)phenyl]carbonyl}- 2,3,4,5-tetrahydro-1,4- benzoxazepin-7-yl)-1,3- benzothiazol-2-amine |
| 357 | 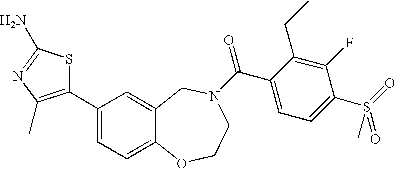 | 5-(4-{[2-ethy]-3-fluoro-4- (methylsulfonyl)phenyl]carbonyl}- 2,3,4,5-tetrahydro-1,4- benzoxazepin-7-yl)-4-methyl- 1,3-thiazol-2-amine |
| 358 | 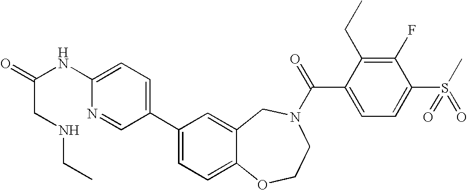 | N²-ethyl-N-[5-(4-{[2-ethyl-3- fluoro-4- (methylsulfonyl)phenyl]carbonyl}- 2,3,4,5-tetrahydro-1,4- benzoxazepin-7-yl)pyridin-2- yl]glycinamide |
| 359 | 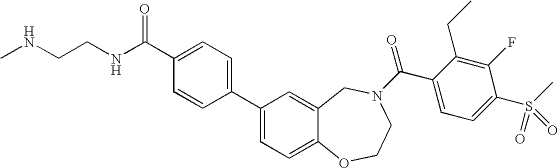 | 4-(4-{[2-ethyl-3-fluoro-4- (methylsulfonyl)phenyl]carbonyl}- 2,3,4,5-tetrahydro-1,4- benzoxazepin-7-yl)-N-[2- (methylamino)ethyl]benzamide |
| 360 | 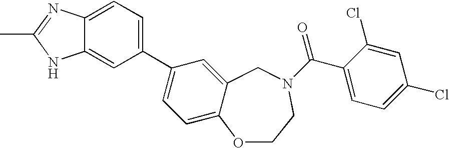 | 4-[(2,4- dichlorophenyl)carbonyl]-7-(2- methyl-1H-benzimidazol-6-yl)- 2,3,4,5-tetrahydro-1,4- benzoxazepine |
| 361 | 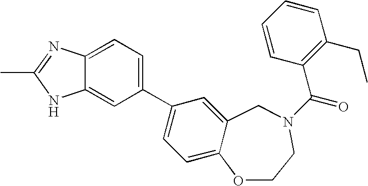 | 4-[(2-ethylphenyl)carbonyl]-7- (2-methyl-1H-benzimidazol-6- yl)-2,3,4,5-tetrahydro-1,4- benzoxazepine |
| 362 | 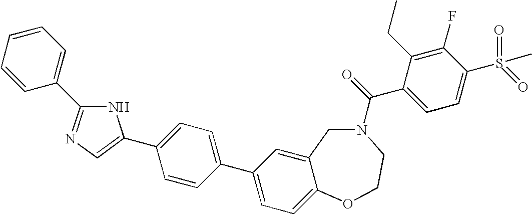 | 4-{[2-ethyl-3-fluoro-4- (methylsulfonyl)phenyl]carbonyl}- 7-[4-(2-phenyl-1H-imidazol-5- yl)phenyl]-2,3,4,5-tetrahydro- 1,4-benzoxazepine |
| 363 | 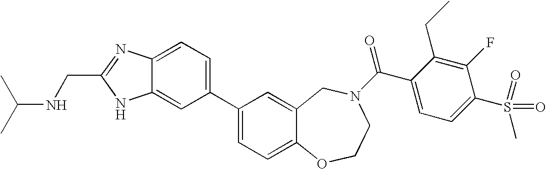 | N-{[6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2- yl]methyl}propan-2-amine |
| 364 | 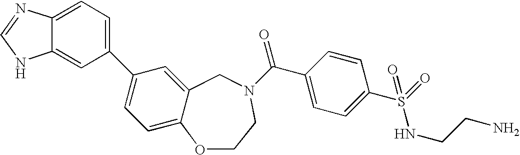 | N-(2-aminoethyl)-4-{[7-(1H- benzimidazol-6-yl)-2,3-dihydro- 1,4-benzoxazepin-4(5H)- yl]carbonyl}benzenesulfonamide |
| 365 | 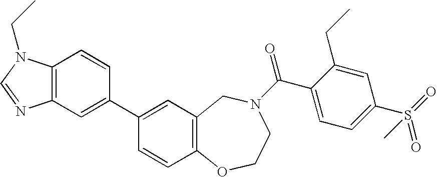 | 7-(1-ethyl-1H-benzimidazol-5- yl)-4-{[2-ethyl-4- (methylsulfonyl)phenyl]carbonyl}- 2,3,4,5-tetrahydro-1,4- benzoxazepine |
| 366 | 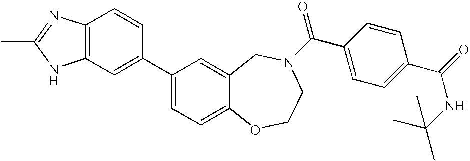 | N-(1,1-dimethylethyl)-4-{[7-(2- methyl-1H-benzimidazol-6-yl)- 2,3-dihydro-1,4-benzoxazepin- 4(5H)-yl]carbonyl}benzamide |
| 367 | 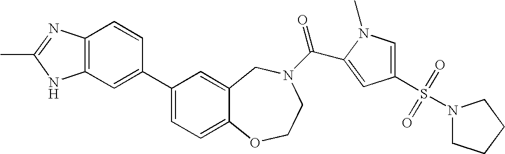 | 7-(2-methyl-1H-benzimidazol-6- yl)-4-{[1-methyl-4-(pyrrolidin- 1-ylsulfonyl)-1H-pyrrol-2- yl]carbonyl}-2,3,4,5-tetrahydro- 1,4-benzoxazepine |
| 368 | 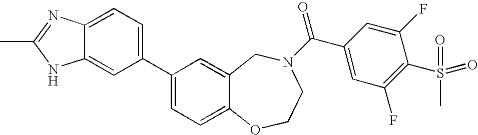 | 4-{[3,5-difluoro-4- (methylsulfonyl)phenyl]carbonyl}- 7-(2-methyl-1H-benzimidazol- 6-yl)-2,3,4,5-tetrahydro-1,4- benzoxazepine |
| 369 | 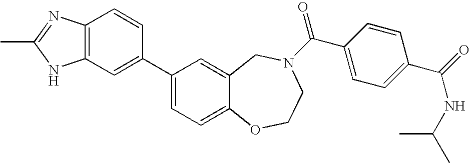 | 4-{[7-(2-methyl-1H- benzimidazol-6-yl)-2,3-dihydro- 1,4-benzoxazepin-4(5H)- yl]carbonyl}-N-(1- methylethyl)benzamide |
| 370 | 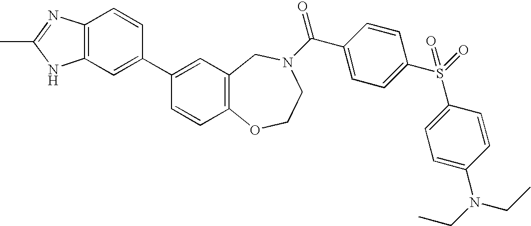 | N,N-diethyl-4-[(4-{[7-(2-methyl- 1H-benzimidazol-6-yl)-2,3- dihydro-1,4-benzoxazepin- 4(5H- yl]carbonyl}phenyl)sulfonyl] aniline |
| 371 | 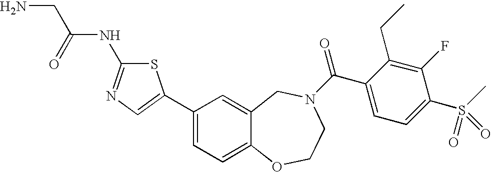 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]glycinamide |
| 372 | 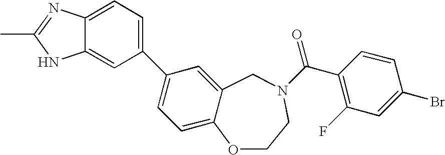 | 4-[(4-bromo-2- fluorophenyl)carbonyl]-7-(2- methyl-1H-benzimidazol-6-yl)- 2,3,4,5-tetrahydro-1,4- benzoxazepine |
| 373 | 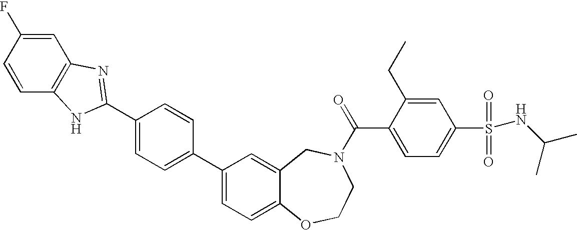 | 3-ethyl-4-({7-[4-(5-fluoro-1H- benzimidazol-2-yl)phenyl]-2,3- dihydro-1,4-benzoxazepin- 4(5H)-yl}carbonyl)-N-(1- methylethyl)benzenesulfonamide |
| 374 | 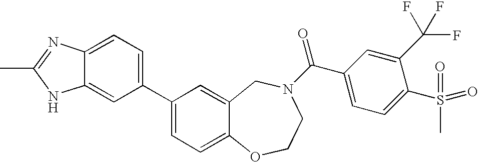 | 7-(2-methyl-1H-benzimidazol-6- yl)-4-{[4-(methylsulfonyl)-3- (trifluoromethyl)phenyl]carbonyl}- 2,3,4,5-tetrahydro-1,4- benzoxazepine |
| 375 | 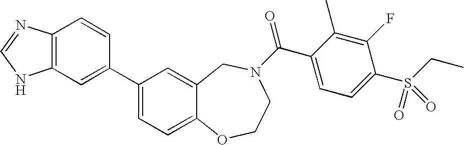 | 7-(1H-benzimidazol-6-yl)-4-{[4- (ethylsulfony])-3-fluoro-2- methylphenyl]carbonyl}-2,3,4,5- tetrahydro-1,4-benzoxazepine |
| 376 | 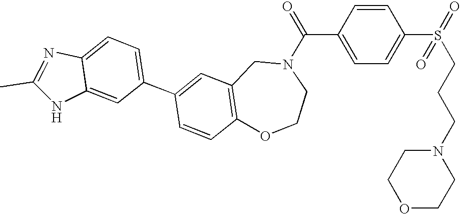 | 7-(2-methyl-1H-benzimidazol-6- yl)-4-({4-[(3-morpholin-4- ylpropyl)sulfonyl]phenyl}carbonyl)- 2,3,4,5-tetrahydro-1,4- benzoxazepine |
| 377 | 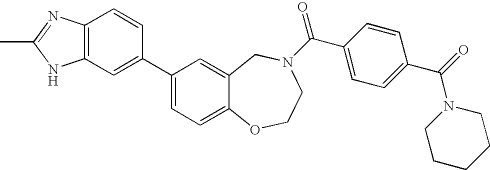 | 7-(2-methyl-1H-benzimidazol-6- yl)-4-{[4-(piperidin-1- ylcarbonyl)phenyl]carbonyl}- 2,3,4,5-tetrahydro-1,4- benzoxazepine |
| 378 | 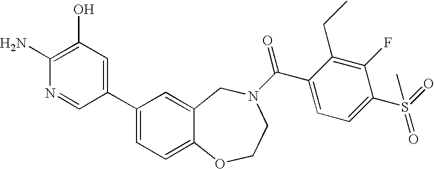 | 2-amino-5-(4-{[2-ethyl-3-fluoro-4- (methylsulfonyl)phenyl]carbonyl}- 2,3,4,5-tetrahydro-1,4- benzoxazepin-7-yl)pyridin-3-ol |
| 379 | 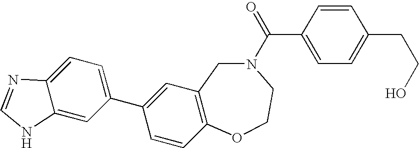 | 2-(4-{[7-(lH-benzimidazol-6- yl)-2,3-dihydro-1,4- benzoxazepin-4(5H)- yl]carbonyl}phenyl)ethanol |
| 380 | 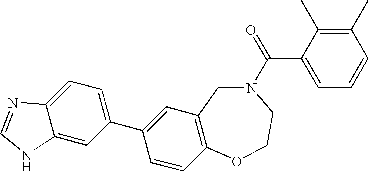 | 7-(1H-benzimidazol-6-yl)-4- [(2,3-dimethylphenyl)carbonyl]- 2,3,4,5-tetrahydro-1,4- benzoxazepine |
| 381 | 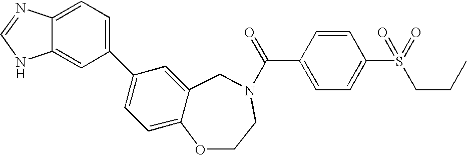 | 7-(1H-benzimidazol-6-yl)-4-{[4- (propylsulfonyl)phenyl]carbonyl}- 2,3,4,5-tetrahydro-1,4- benzoxazepine |
| 382 | 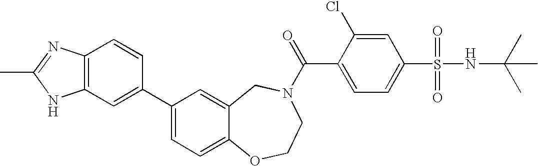 | 3-chloro-N-(1,1-dimethylethyl)- 4-{[7-(2-methyl-1H- benzimidazol-6-yl)-2,3-dihydro- 1,4-benzoxazepin-4(5H)- yl]carbonyl}benzenesulfonamide |
| 383 | 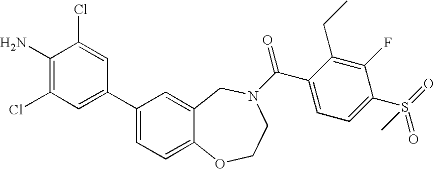 | 2,6-dichloro-4-(4-{[2-ethyl-3- fluoro-4- (methylsulfonyl)phenyl]carbonyl}- 2,3,4,5-tetrahydro-1,4- benzoxazepin-7-yl)aniline |
| 384 | 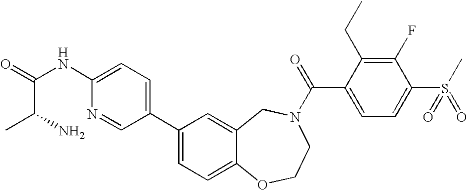 | N-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- D-alaninamide |
| 385 | 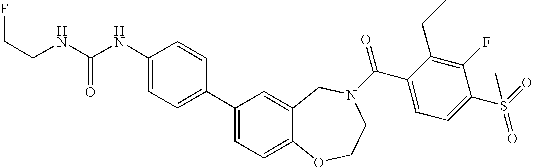 | l-[4-(4-{[2-ethyl-3-fluoro-4- (methylsulfonyl)phenyl]carbonyl}- 2,3,4,5-tetrahydro-1,4- benzoxazepin-7-yl)phenyl]-3-(2- fluoroethyl)urea |
| 386 | 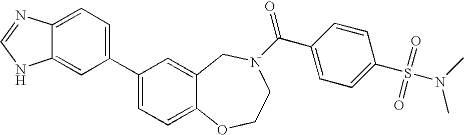 | 4-{[7-(1H-benzimidazol-6-yl)- 2,3-dihydro-1,4-benzoxazepin- 4(5H)-yl]carbonyl}-N,N- dimethylbenzenesulfonamide |
| 387 | 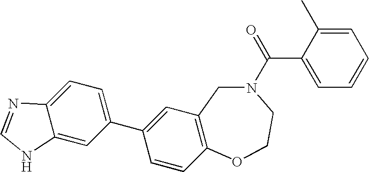 | 7-(1H-benzimidazol-6-yl)-4-[(2- methylphenyl)carbonyl]-2,3,4,5- tetrahydro-1,4-benzoxazepine |
| 388 | 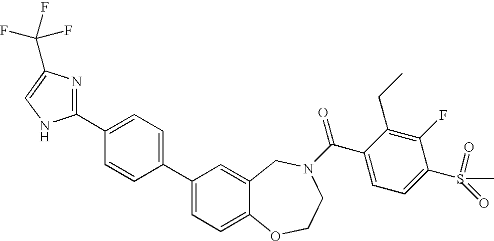 | 4-{[2-ethyl-3-fluoro-4- (methylsulfonyl)phenyl]carbonyl}- 7-{4-[4-(trifluoromethyl)-1H- imidazol-2-yl]phenyl}-2,3,4,5- tetrahydro-1,4-benzoxazepine |
| 389 | 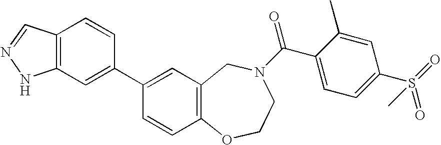 | 7-(1H-indazol-6-yl)-4-{[2- methyl-4- (methylsulfonyl)phenyl]carbonyl}- 2,3,4,5-tetrahydro-1,4- benzoxazepine |
| 390 | 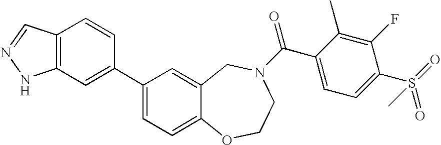 | 4-{[3-fluoro-2-methyl-4- (methylsulfonyl)phenyl]carbonyl}- 7-(1H-indazol-6-yl)-2,3,4,5- tetrahydro-1,4-benzoxazepine |
| 391 | 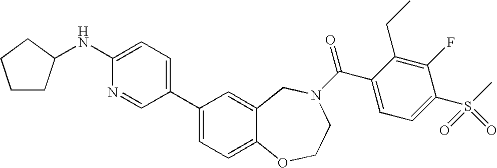 | N-cyclopentyl-5-(4-{[2-ethyl-3- fluoro-4- (methylsulfonyl)phenyl]carbonyl}- 2,3,4,5-tetrahydro-1,4- benzoxazepin-7-yl)pyridin-2- amine |
| 392 | 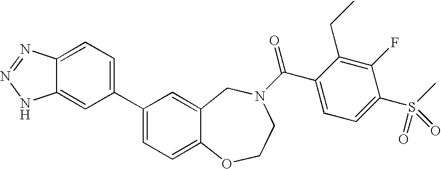 | 7-(1H-1,2,3-benzotriazol-6-yl)- 4-{[2-ethyl-3-fluoro-4- (methylsulfonyl)phenyl]carbonyl}- 2,3,4,5-tetrahydro-1,4- benzoxazepine |
| 393 | 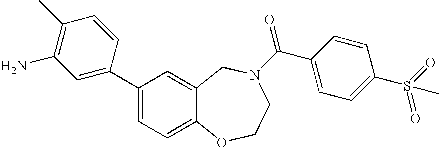 | 2-methyl-5-(4-{[4- (methylsulfonyl)phenyl]carbonyl}- 2,3,4,5-tetrahydro-1,4- benzoxazepin-7-yl)aniline |
| 394 | 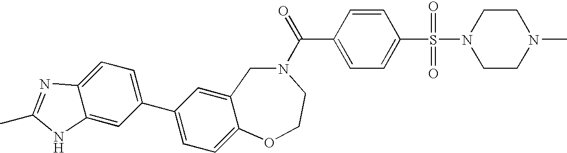 | 7-(2-methyl-1H-benzimidazol-6- yl)-4-({4-[(4-methylpiperazin-1- yl)sulfonyl]phenyl}carbonyl)- 2,3,4,5-tetrahydro-1,4- benzoxazepine |
| 395 | 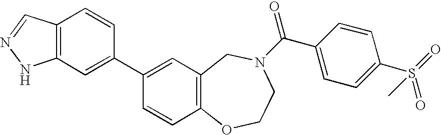 | 7-(1H-indazol-6-yl)-4-{[4- (methylsulfonyl)phenyl]carbonyl}- 2,3,4,5-tetrahydro-1,4- benzoxazepine |
| 396 | 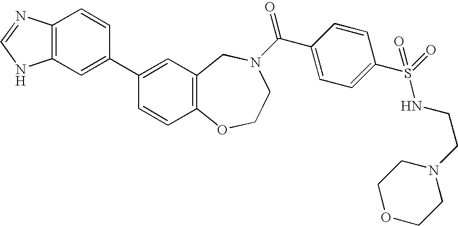 | 4-{[7-(1H-benzimidazol-6-yl)- 2,3-dihydro-1,4-benzoxazepin- 4(5H)-yl]carbonyl}-N-(2- morpholin-4- ylethyl)benzenesulfonamide |
| 397 | 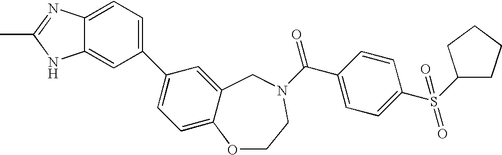 | 4-{[4- (cyclopentylsulfonyl)phenyl] carbonyl}-7-(2-methyl-1H- benzimidazol-6-yl)-2,3,4,5- tetrahydro-1,4-benzoxazepine |
| 398 | 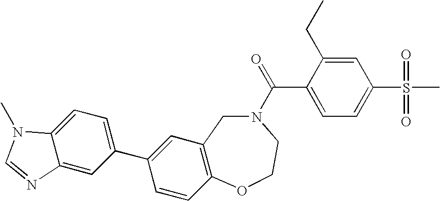 | 4-{[2-ethyl-4- (methylsulfonyl)phenyl]carbonyl}- 7-(1-methyl-1H-benzimidazol- 5-yl)-2,3,4,5-tetrahydro-1,4- benzoxazepine |
| 399 | 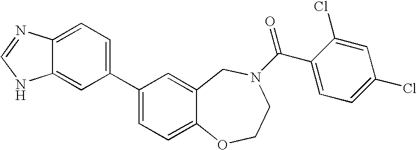 | 7-(1H-benzimidazol-6-yl)-4- [(2,4-dichlorophenyl)carbonyl]- 2,3,4,5-tetrahydro-1,4- benzoxazepine |
| 400 | 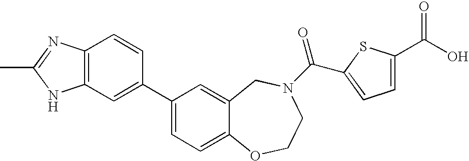 | 5-{[7-(2-methyl-1H- benzimidazol-6-yl)-2,3-dihydro- 1,4-benzoxazepin-4(5H)- yl]carbonyl}thiophene-2- carboxylic acid |
| 401 | 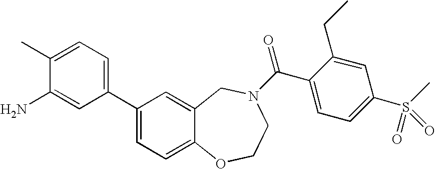 | 5-(4-{[2-ethyl-4- (methylsulfonyl)phenyl]carbonyl}- 2,3,4,5-tetrahydro-1,4- benzoxazepin-7-yl)-2- methylaniline |
| 402 | 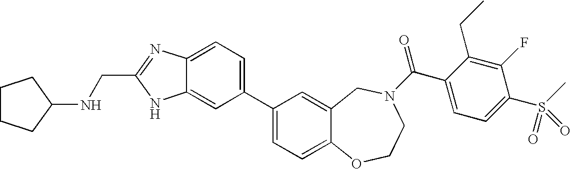 | N-{[6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2- yl]methyl}cyclopentanamine |
| 403 | 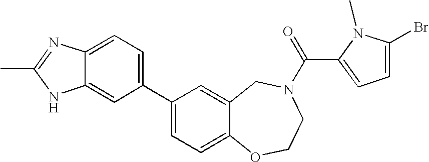 | 4-[(5-bromo-1-methyl-1H- pyrrol-2-yl)carbonyl]-7-(2- methyl-1H-benzimidazol-6-yl)- 2,3,4,5-tetrahydro-1,4- benzoxazepine |
| 404 | 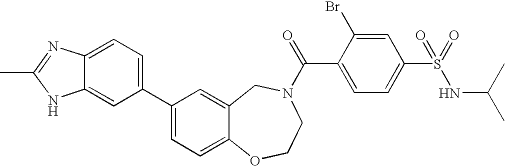 | 3-bromo-4-{[7-(2-methyl-1H- benzimidazol-6-yl)-2,3-dihydro- 1,4-benzoxazepin-4(5H)- yl]carbonyl}-N-(1- methylethyl)benzenesulfonamide |
| 405 | 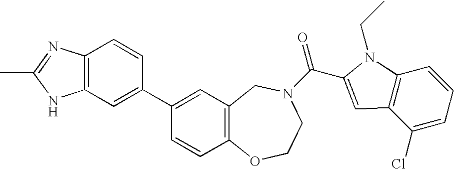 | 4-[(4-chloro-1-ethyl-1H-indol-2- yl)carbonyl]-7-(2-methyl-1H- benzimidazol-6-yl)-2,3,4,5- tetrahydro-1,4-benzoxazepine |
| 406 | 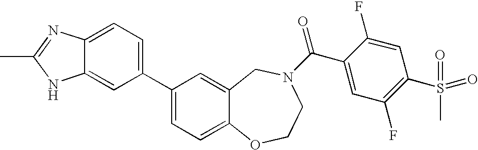 | 4-{[2,5-difluoro-4- (methylsulfonyl)phenyl]carbonyl}- 7-(2-methyl-1H-benzimidazol- 6-yl)-2,3,4,5-tetrahydro-1,4- benzoxazepine |
| 407 | 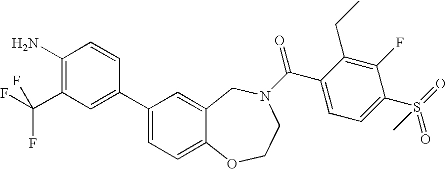 | 4-(4-{[2-ethyl-3-fluoro-4- (methylsulfonyl)phenyl]carbonyl}- 2,3,4,5-tetrahydro-1,4- benzoxazepin-7-yl)-2- (trifluoromethyl)aniline |
| 408 | 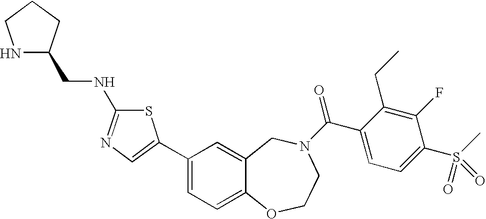 | 5-(4-{[2-ethyl-3-fluoro-4- (methylsulfonyl)phenyl]carbonyl}- 2,3,4,5-tctrahydro-1,4- benzoxazepin-7-yl)-N-[(2S)- pyrrolidin-2-ylmethyl]-1,3- thiazol-2-amine |
| 409 | 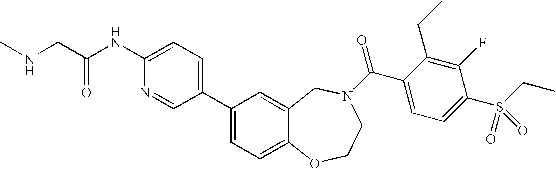 | N-[5-(4-{[2-ethyl-4- (ethylsulfonyl)-3- fluorophenyl]carbonyl}-2,3,4,5- tetrahydro-1,4-benzoxazepin-7- yl)pyridin-2-yl]-N²- methylglycinamide |
| 410 | 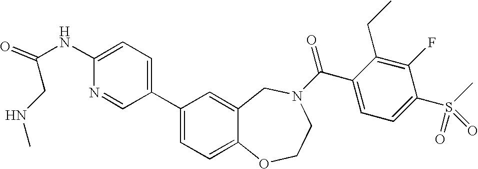 | N-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- N²-methylglycinamide |
| 411 | 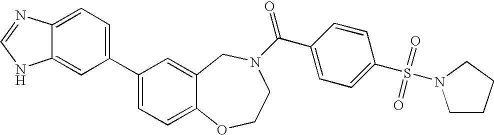 | 7-(1H-benzimidazol-6-yl)-4-{[4- (pyrrolidin-1- ylsulfonyl)phenyl]carbonyl}- 2,3,4,5-tetrahydro-1,4- benzoxazepine |
| 412 | 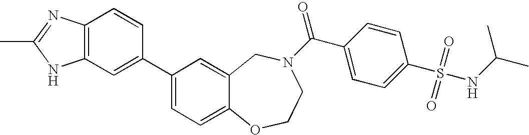 | 4-{[7-(2-methyl-1H- benzimidazol-6-yl)-2,3-dihydro- 1,4-benzoxazepin-4(5H)- yl]carbonyl}-N-(1- methylethyl)benzenesulfonamide |
| 413 | 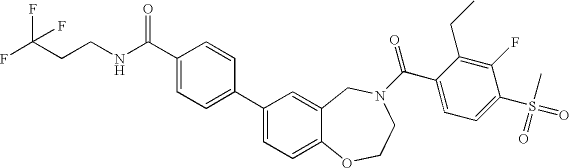 | 4-(4-{[2-ethyl-3-fluoro-4- (methylsulfonyl)phenyl]carbonyl}- 2,3,4,5-tetrahydro-1,4- benzoxazepin-7-yl)-N-(3,3,3- trifluoropropyl)benzamide |
| 414 | 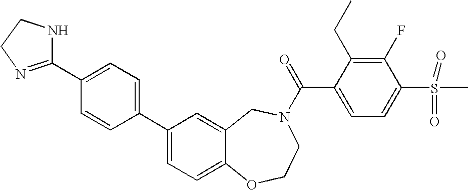 | 7-[4-(4,5-dihydro-1H-imidazol- 2-yl)phenyl]-4-{[2-ethyl-3- fluoro-4- (methylsulfonyl)phenyl]carbonyl}- 2,3,4,5-tetrahydro-1,4- benzoxazepine |
| 415 | 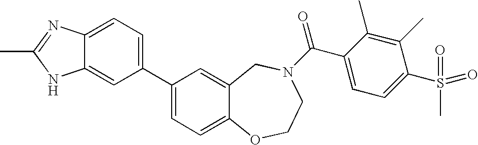 | 4-{[2,3-dimethyl-4- (methylsulfonyl)phenyl]carbonyl}- 7-(2-methyl-1H-benzimidazol- 6-yl)-2,3,4,5-tetrahydro-1,4- benzoxazepine |
| 416 | 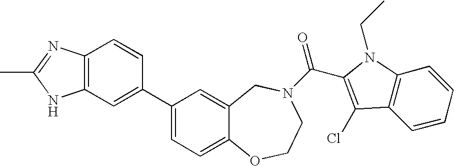 | 4-[(3-chloro-1-ethyl-1H-indol-2- yl)carbonyl]-7-(2-methyl-1H- benzimidazol-6-yl)-2,3,4,5- tetrahydro-1,4-benzoxazepine |
| 417 | 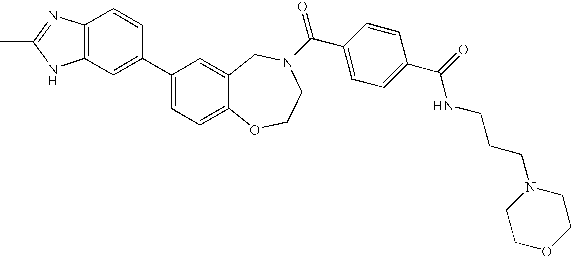 | 4-{[7-(2-methyl-1H- benzimidazol-6-yl)-2,3-dihydro- 1,4-benzoxazepin-4(5H)- yl]carbonyl}-N-(3-morpholin-4- ylpropyl)benzamide |
| 418 | 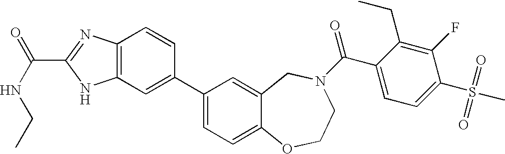 | N-ethyl-6-(4-{[2-ethyl-3-fluoro-4- (methylsulfonyl)phenyl]carbonyl}- 2,3,4,5-tetrahydro-1,4- benzoxazepin-7-yl)-1H- benzimidazole-2-carboxamide |
| 419 | 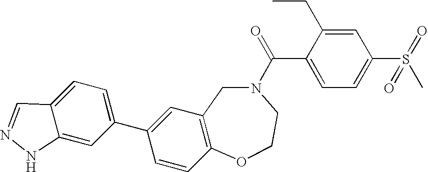 | 4-{[2-ethyl-4- (methylsulfonyl)phenyl]carbonyl}- 7-(1H-indazol-6-yl)-2,3,4,5- tetrahydro-1,4-benzoxazepine |
| 420 | 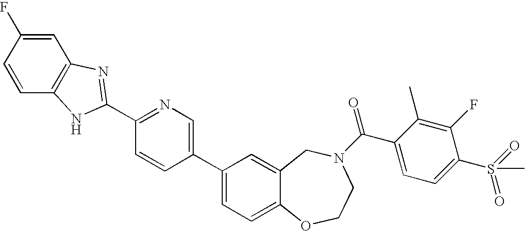 | 7-[6-(5-fluoro-1H-benzimidazol- 2-yl)pyridin-3-yl]-4-{[3-fluoro- 2-methyl-4- (methylsulfonyl)phenyl]carbonyl}- 2,3,4,5-tetrahydro-1,4- benzoxazepine |
| 421 | 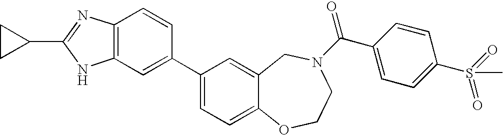 | 7-(2-cyclopropyl-1H- benzimidazol-6-yl)-4-{[4- (methylsulfonyl)phenyl]carbonyl}- 2,3,4,5-tetrahydro-1,4- benzoxazepine |
| 422 | 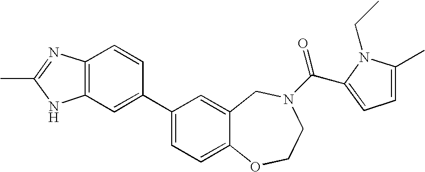 | 4-[(1-ethyl-5-methyl-1H-pyrrol- 2-yl)carbonyl]-7-(2-methyl-1H- benzimidazol-6-yl)-2,3,4,5- tetrahydro-1,4-benzoxazepine |
| 423 | 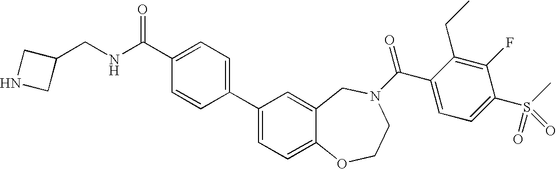 | N-(azetidin-3-ylmethyl)-4-(4- {[2-ethyl-3-fluoro-4- (methylsulfonyl)phenyl]carbonyl}- 2,3,4,5-tetrahydro-1,4- benzoxazepin-7-yl)benzamide |
| 424 | 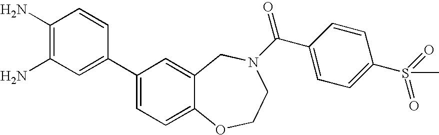 | 4-(4-{[4- (methylsulfonyl)phenyl]carbonyl}- 2,3,4,5-tetrahydro-1,4- benzoxazepin-7-yl)benzene-1,2- diamine |
| 425 | 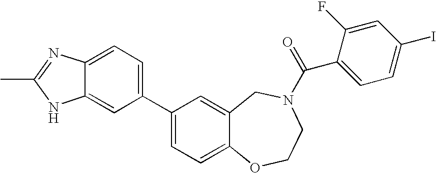 | 4-[(2-fluoro-4- iodophenyl)carbonyl]-7-(2- methyl-1H-benzimidazol-6-yl)- 2,3,4,5-tetrahydro-1,4- benzoxazepine |
| 426 | 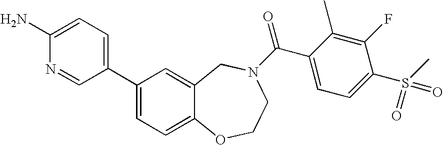 | 5-(4-{[3-fluoro-2-methyl-4- (methylsulfonyl)phenyl]carbonyl}- 2,3,4,5-tetrahydro-1,4- benzoxazepin-7-yl)pyridin-2- amine |
| 427 | 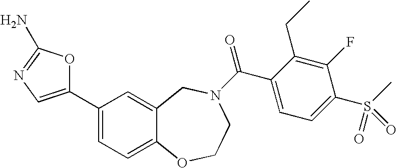 | 5-(4-{[2-ethyl-3-fluoro-4- (methylsulfonyl)phenyl]carbonyl}- 2,3,4,5-tetrahydro-1,4- benzoxazepin-7-yl)-1,3-oxazol- 2-amine |
| 428 | 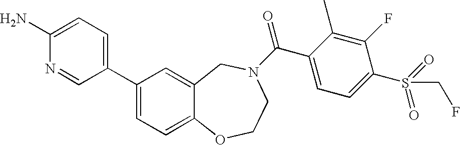 | 5-[4-({3-fluoro-4- [(fluoromethyl)sulfonyl]-2- methylphenyl}carbonyl)-2,3,4,5- tetrahydro-1,4-benzoxazepin-7- yl]pyridin-2-amine |
| 429 | 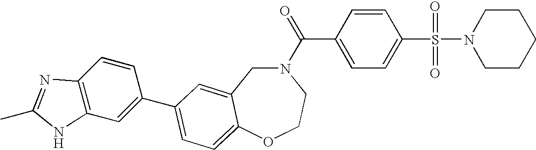 | 7-(2-methyl-1H-benzimidazol-6- yl)-4-{[4-(piperidin-1- ylsulfonyl)phenyl]carbonyl}- 2,3,4,5-tetrahydro-1,4- benzoxazepine |
| 430 | 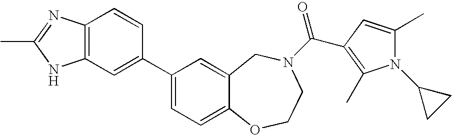 | 4-[(1-cyclopropyl-2,5-dimethyl- 1H-pyrrol-3-yl)carbonyl]-7-(2- methyl-1H-benzimidazol-6-yl)- 2,3,4,5-tetrahydro-1,4- benzoxazepine |
| 431 | 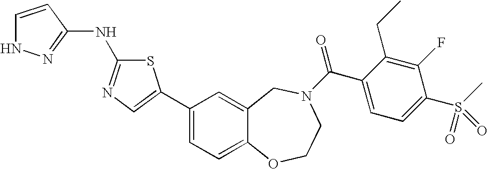 | 5-(4-{[2-ethyl-3-fluoro-4- (methylsulfonyl)phenyl]carbonyl}- 2,3,4,5-tetrahydro-1,4- benzoxazepin-7-yl)-N-1H- pyrazol-3-yl-1,3-thiazol-2-amine |
| 432 | 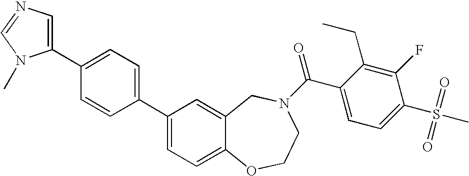 | 4-{[2-ethyl-3-fluoro-4- (methylsulfonyl)phenyl]carbonyl}- 7-[4-(1-methyl-1H-imidazol-5- yl)pheny1]-2,3,4,5-tetrahydro- 1,4-benzoxazepine |
| 433 | 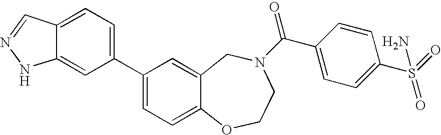 | 4-{[7-(1H-indazol-6-yl)-2,3- dihydro-1,4-benzoxazepin- 4(5H)- yl]carbonyl}benzenesulfonamide |
| 434 | 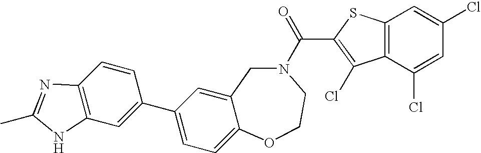 | 7-(2-methyl-1H-benzimidazol-6- yl)-4-[(3,4,6-trichloro-1- benzothien-2-yl)carbonyl]- 2,3,4,5-tetrahydro-1,4- benzoxazepine |
| 435 | 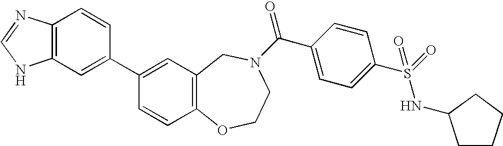 | 4-{[7-(1H-benzimidazol-6-yl)- 2,3-dihydro-1,4-benzoxazepin- 4(5H)-yl]carbonyl}-N- cyclopentylbenzenesulfonamide |
| 436 | 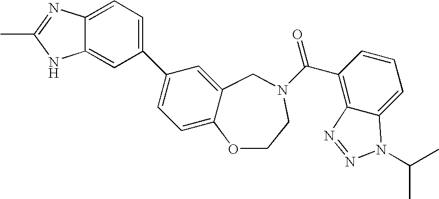 | 7-(2-methyl-1H-benzimidazol-6- yl)-4-{[1-(1-methylethyl)-1H- 1,2,3-benzotriazol-5- yl]carbonyl}-2,3,4,5-tetrahydro- 1,4-benzoxazepine |
| 437 | 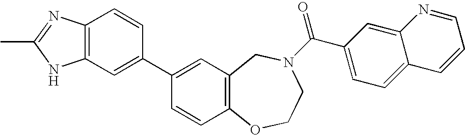 | 7-(2-methyl-1H-benzimidazol-6- yl)-4-(quinolin-7-ylcarbonyl)- 2,3,4,5-tetrahydro-1,4- benzoxazepine |
| 438 | 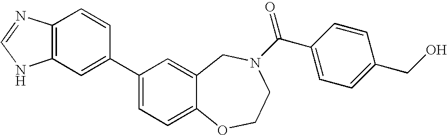 | (4-{[7-(1H-benzimidazol-6-yl)- 2,3-dihydro-1,4-benzoxazepin- 4(5H)- yl]carbonyl}phenyl)methanol |
| 439 | 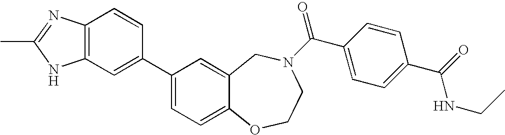 | N-ethyl-4-{[7-(2-methyl-1H- benzimidazol-6-yl)-2,3-dihydro- 1,4-benzoxazepin-4(5H)- yl]carbonyl}benzamide |
| 440 | 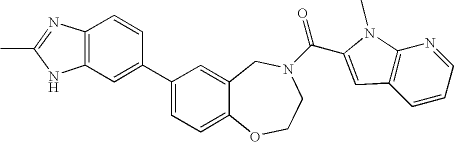 | 7-(2-methyl-1H-benzimidazol-6- yl)-4-[(1-methyl-1H-pyrrolo[2,3- b]pyridin-2-yl)carbonyl]-2,3,4,5- tetrahydro-1,4-benzoxazepine |
| 441 | 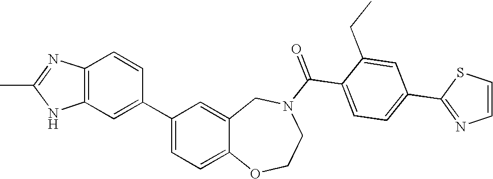 | 4-{[2-ethyl-4-(1,3-thiazol-2- yl)phenyl]carbonyl}-7-(2- methyl-1H-benzimidazol-6-yl)- 2,3,4,5-tetrahydro-1,4- benzoxazepine |
| 442 | 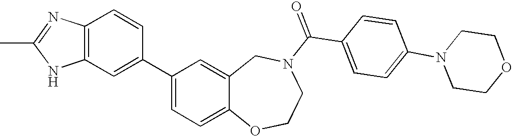 | 7-(2-methyl-1H-benzimidazol-6- yl)-4-[(4-morpholin-4- ylphenyl)carbonyl]-2,3,4,5- tetrahydro-1,4-benzoxazepine |
| 443 | 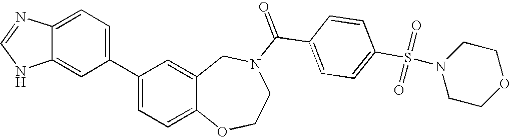 | 7-(1H-benzimidazol-6-yl)-4- {[4-(morpholin-4- ylsulfonyl)phenyl]carbonyl}- 2,3,4,5-tetrahydro-1,4- benzoxazepine |
| 444 | 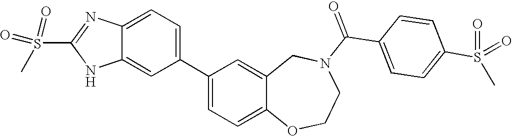 | 7-[2-(methylsulfonyl)-1H- benzimidazol-6-yl]-4- (methylsulfonyl)phenyl]carbonyl}- 2,3,4,5-tetrahydro-1,4- benzoxazepine |
| 445 | 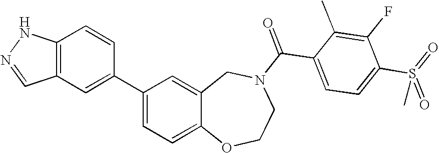 | 4-{[3-fluoro-2-methyl-4- (methylsulfonyl)phenyl]carbonyl}- 7-(1H-indazol-5-yl)-2,3,4,5- tetrahydro-1,4-benzoxazepine |
| 446 | 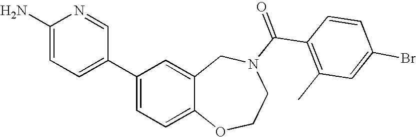 | 5-{4-[(4-bromo-2- methylphenyl)carbonyl]-2,3,4,5- tetrahydro-1,4-benzoxazepin-7- yl}pyridin-2-amine |
| 447 | 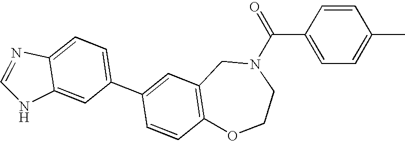 | 7-(1H-benzimidazol-6-yl)-4-[(4- methylphenyl)carbonyl]-2,3,4,5- tetrahydro-1,4-benzoxazepine |
| 448 | 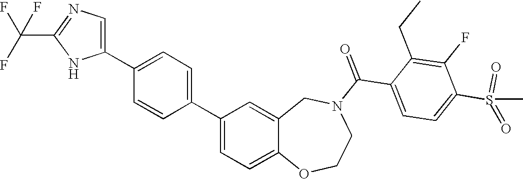 | 4-{[2-ethyl-3-fluoro-4- (methylsulfonyl)phenyl]carbonyl}- 7-{4-[2-(trifluoromethyl)-1H- imidazol-5-yl]phenyl}-2,3,4,5- tetrahydro-1,4-benzoxazepine |
| 449 | 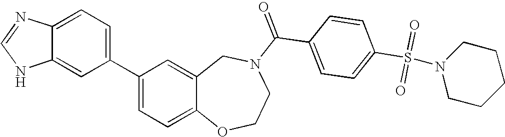 | 7-(1H-benzimidazol-6-yl)-4-{[4- (piperidin-1- ylsulfonyl)phenyl]carbonyl}- 2,3,4,5-tetrahydro-1,4- benzoxazepine |
| 450 | 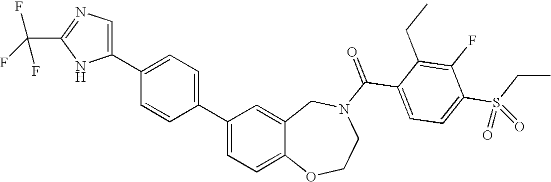 | 4-{[2-ethyl-4-(ethylsulfonyl)-3- fluorophenyl]carbonyl}-7-{4-[2- (trifluoromethyl)-1H-imidazol- 5-yl]phenyl}-2,3,4,5-tetrahydro- 1,4-benzoxazepine |
| 451 | 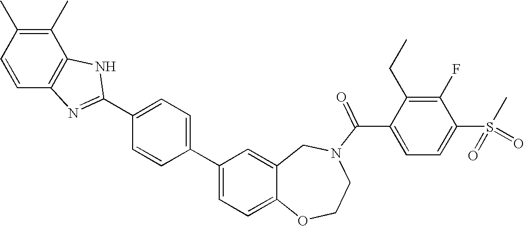 | 7-[4-(6,7-dimethyl-1H- benzimidazol-2-yl)phenyl]-4- {[2-ethyl-3-fluoro-4- (methylsulfonyl)phenyl]carbonyl}- 2,3,4,5-tetrahydro-1,4- benzoxazepine |
| 452 | 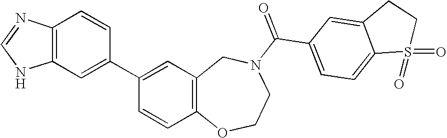 | 7-(1H-benzimidazol-6-yl)-4- [(1,1-dioxido-2,3-dihydro-1- benzothien-5-yl)carbonyl]- 2,3,4,5-tetrahydro-1,4- benzoxazepine |
| 453 | 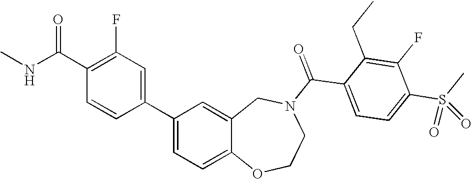 | 4-(4-{[2-ethyl-3-fluoro-4- (methylsulfonyl)phenyl]carbonyl}- 2,3,4,5-tetrahydro-1,4- benzoxazepin-7-yl)-2-fluoro-N- methylbenzamide |
| 454 | 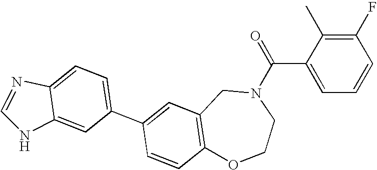 | 7-(1H-benzimidazol-6-yl)-4-[(3- fluoro-2- methylphenyl)carbonyl]-2,3,4,5- tetrahydro-1,4-benzoxazepine |
| 455 | 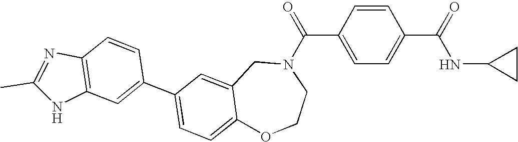 | N-cyclopropyl-4-{[7-(2-methyl- 1H-benzimidazol-6-yl)-2,3- dihydro-1,4-benzoxazepin- 4(5H)-yl]carbonyl}benzamide |
| 456 | 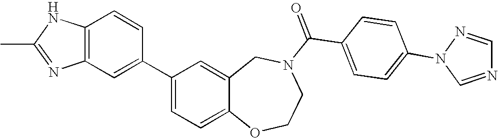 | 7-(2-methyl-1H-benzimidazol-5- yl)-4-{[4-(1H-1,2,4-triazol-1- yl)phenyl]carbonyl}-2,3,4,5- tetrahydro-1,4-benzoxazepine |
| 457 | 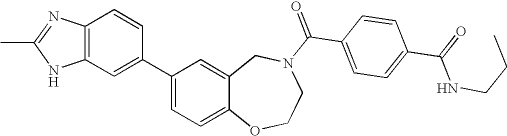 | 4-{[7-(2-methyl-1H- benzimidazol-6-yl)-2,3-dihydro- 1,4-benzoxazepin-4(5H)- yl]carbonyl}-N- propylbenzamide |
| 458 | 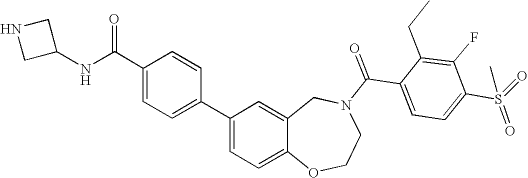 | N-azetidin-3-yl-4-(4-{[2-ethyl-3- fluoro-4- (methylsulfonyl)phenyl]carbonyl}- 2,3,4,5-tetrahydro-1,4- benzoxazepin-7-yl)benzamide |
| 459 | 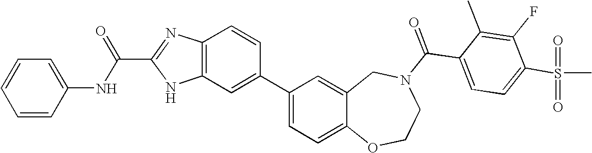 | 6-(4-{[3-fluoro-2-methyl-4- (methylsulfonyl)phenyl]carbonyl}- 2,3,4,5-tetrahydro-1,4- benzoxazepin-7-yl)- N-phenyl-1H-benzimidazole-2- carboxamide |
| 460 | 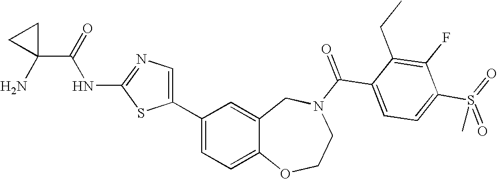 | 1-amino-N-[5-(4-{[2-ethyl-3- fluoro-4- (methylsulfonyl)phenyl]carbonyl}- 2,3,4,5-tetrahydro-1,4- benzoxazepin-7-yl)-1,3-thiazol- 2-yl]cyclopropanecarboxamide |
| 461 | 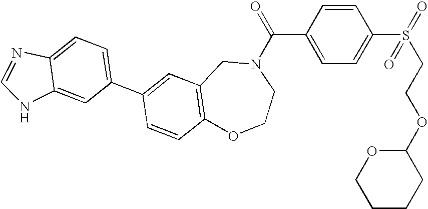 | 7-(1H-benzimidazol-6-yl)-4-[(4- {[2-(tetrahydro-2H-pyran-2- yloxy)ethyl]sulfonyl}phenyl) carbonyl]-2,3,4,5-tetrahydro-1,4- benzoxazepine |
| 462 | 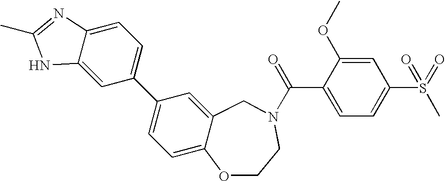 | 7-(2-methyl-1H-benzimidazol-6- yl)-4-{[2-(methyloxy)-4- (methylsulfonyl)phenyl]carbonyl}- 2,3,4,5-tetrahydro-1,4- benzoxazepine |
| 463 | 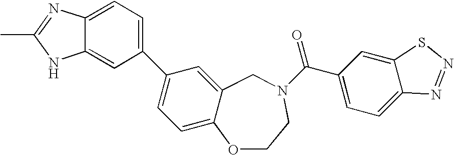 | 4-(1,2,3-benzothiadiazol-6- ylcarbonyl)-7-(2-methyl-1H- benzimidazol-6-yl)-2,3,4,5- tetrahydro-1,4-benzoxazepine |
| 464 | 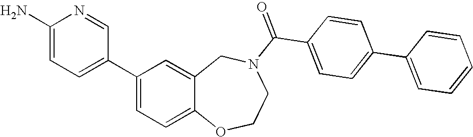 | 5-[4-(biphenyl-4-ylcarbonyl)- 2,3,4,5-tetrahydro-1,4- benzoxazepin-7-yl]pyridin-2- amine |
| 465 | 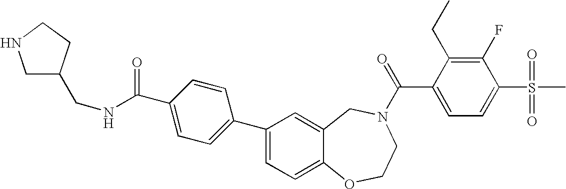 | 4-(4-{[2-ethyl-3-fluoro-4- (methylsulfonyl)phenyl]carbonyl}- 2,3,4,5-tetrahydro-1,4- benzoxazepin-7-yl)-N- (pyrrolidin-3- ylmethyl)benzamide |
| 466 | 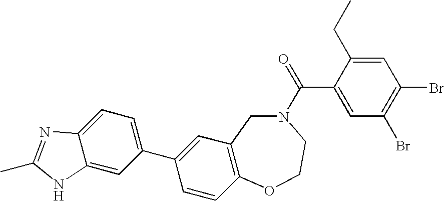 | 4-[(4,5-dibromo-2- ethylphenyl)carbonyl]-7-(2- methyl-1H-benzimidazol-6-yl)- 2,3,4,5-tetrahydro-1,4- benzoxazepine |
| 467 | 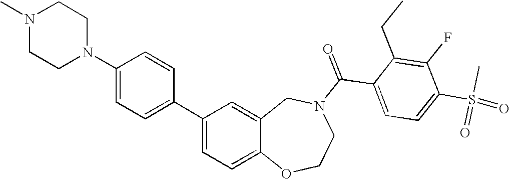 | 4-{[2-ethyl-3-fluoro-4- (methylsulfonyl)phenyl]carbonyl}- 7-[4-(4-methylpiperazin-1- yl)phenyl]-2,3,4,5-tetrahydro- 1,4-benzoxazepine |
| 468 | 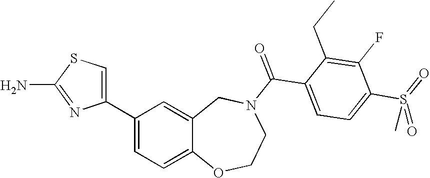 | 4-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-amine |
| 469 | 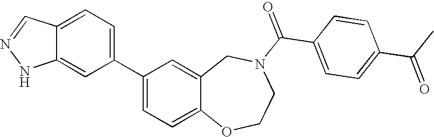 | l-(4-{[7-(1H-indazol-6-yl)-2,3- dihydro-1,4-benzoxazepin- 4(5H)- yl]carbonyl}phenyl)ethanone |
| 470 | 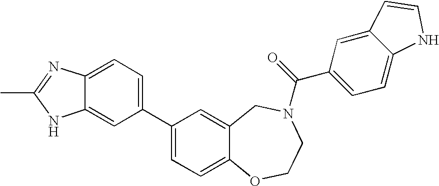 | 4-(1H-indol-5-ylcarbonyl)-7-(2- methyl-1H-benzimidazol-6-yl)- 2,3,4,5-tetrahydro-1,4- benzoxazepine |
| 471 | 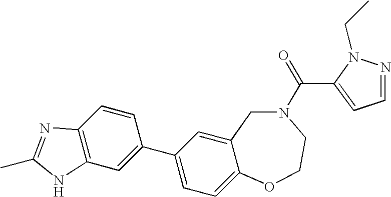 | 4-[(1-ethyl-1H-pyrazol-5- yl)carbonyl]-7-(2-methyl-1H- benzimidazol-6-yl)-2,3,4,5- tetrahydro-1,4-benzoxazepine |
| 472 | 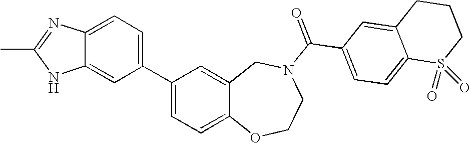 | 4-[(1,1-dioxido-3,4-dihydro-2H- 1-benzothiopyran-6- yl)carbonyl]-7-(2-methyl-1H- benzimidazol-6-yl)-2,3,4,5- tetrahydro-1,4-benzoxazepine |
| 473 | 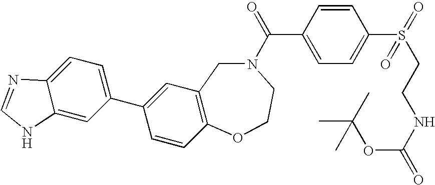 | 1,1-dimethylethyl{2-[(4-{[7- (1H-benzimidazol-6-yl)-2,3- dihydro-1,4-benzoxazepin- 4(5H)- yl]carbonyl}phenyl)sulfonyl] ethyl}carbamate |
| 474 | 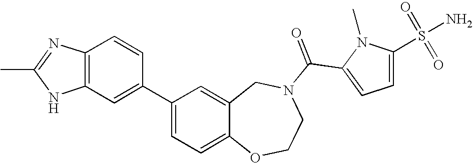 | 1-methyl-5-{[7-(2-methyl-1H- benzimidazol-6-yl)-2,3-dihydro- 1,4-benzoxazepin-4(5H)- yl]carbonyl}-1H-pyrrole-2- sulfonamide |
| 475 | 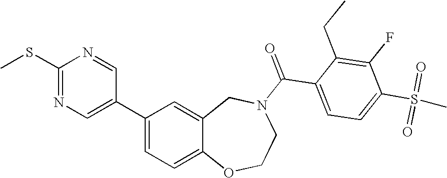 | 4-{[2-ethyl-3-fluoro-4- (methylsulfonyl)phenyl]carbonyl}- 7-[2-(methylthio)pyrimidin-5- yl]-2,3,4,5-tetrahydro-1,4- benzoxazepine |
| 476 | 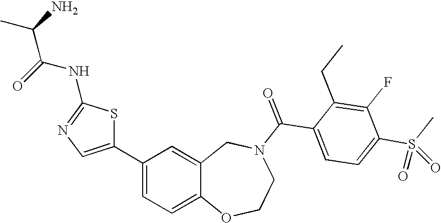 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]-D-alaninamide |
| 477 | 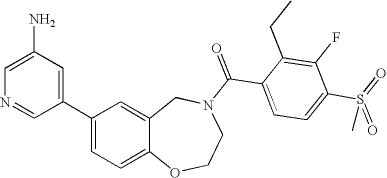 | 5-(4-{[2-ethyl-3-fluoro-4- (methylsulfonyl)phenyl]carbonyl}- 2,3,4,5-tetrahydro-1,4- benzoxazepin-7-yl)pyridin-3-amine |
| 478 | 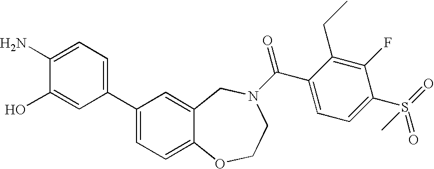 | 2-amino-5-(4-{[2-ethyl-3-fluoro-4- (methylsulfonyl)phenyl]carbonyl}- 2,3,4,5-tetrahydro-1,4- benzoxazepin-7-yl)phenol |
| 479 | 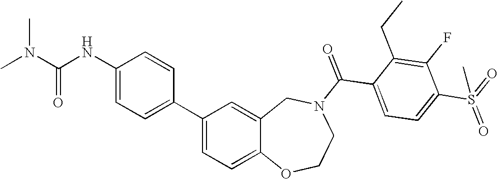 | 3-[4-(4-{[2-ethyl-3-fluoro-4- (methylsulfonyl)phenyl]carbonyl}- 2,3,4,5-tetrahydro-1,4- benzoxazepin-7-yl)phenyl]-1,1- dimethylurea |
| 480 | 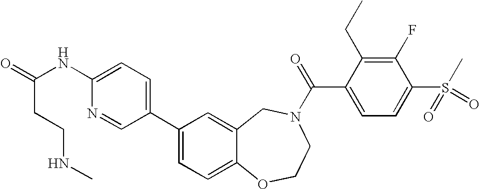 | N-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- N³-methyl-beta-alaninamide |
| 481 | 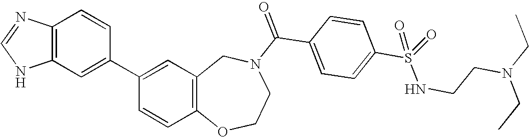 | 4-{[7-(1H-benzimidazol-6-yl)- 2,3-dihydro-1,4-benzoxazepin- 4(5H)-yl]carbonyl]-N-[2- (diethylamino)ethyl] benzenesulfonamide |
| 482 | 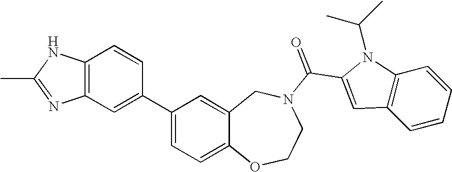 | 7-(2-methyl-1H-benzimidazol-5- yl)-4-{[1-(1-methylethyl)-1H- indol-2-yl]carbonyl}-2,3,4,5- tetrahydro-1,4-benzoxazepine |
| 483 | 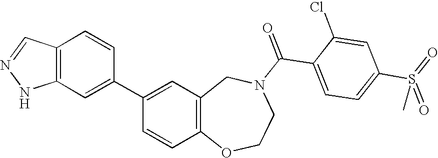 | 4-{[2-chloro-4- (methylsulfonyl)phenyl]carbonyl}- 7-(1H-indazol-6-yl)-2,3,4,5- tetrahydro-1,4-benzoxazepine |
| 484 | 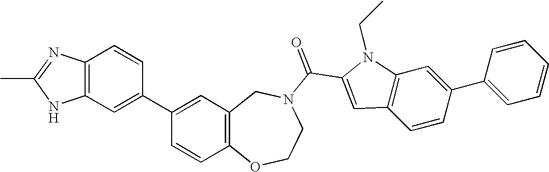 | 4-[(1-ethyl-6-phenyl-1H-indol- 2-yl)carbonyl]-7-(2-methyl-1H- benzimidazol-6-yl)-2,3,4,5- tetrahydro-1,4-benzoxazepine |
| 485 | 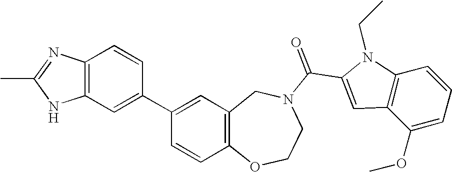 | 4-{[1-ethyl-4-(methyloxy)-1H- indol-2-yl]carbonyl}-7-(2- methyl-1H-benzimidazol-6-yl)- 2,3,4,5-tetrahydro-1,4- benzoxazepine |
| 486 | 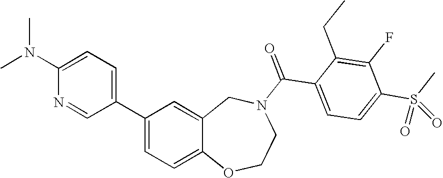 | 5-(4-{[2-ethyl-3-fluoro-4- (methylsulfonyl)phenyl]carbonyl}- 2,3,4,5-tetrahydro-1,4- benzoxazepin-7-yl)-N,N- dimethylpyridin-2-amine |
| 487 | 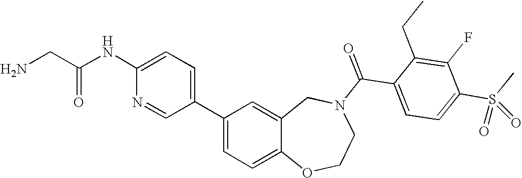 | N-[5-(4-{[2-ethyl-3-fluoro-4- (methylsulfonyl)phenyl]carbonyl}- 2,3,4,5-tetrahydro-1,4- benzoxazepin-7-yl)pyridin-2- yl]glycinamide |
| 488 | 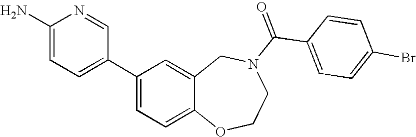 | 5-{4-[(4- bromophenyl)carbonyl]-2,3,4,5- tetrahydro-1,4-benzoxazepin-7- yl}pyridin-2-amine |
| 489 | 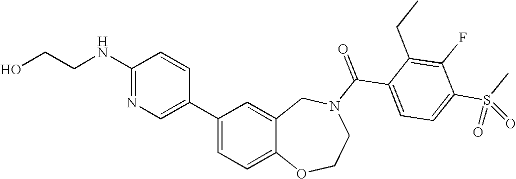 | 2-{[5-(4-{[2-ethyl-3-fluoro-4- (methylsulfonyl)phenyl]carbonyl}- 2,3,4,5-tetrahydro-1,4- benzoxazepin-7-yl)pyridin-2- yl]amino}ethanol |
| 490 | 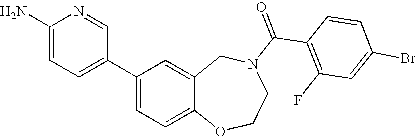 | 5-{4-[(4-bromo-2- fluorophenyl)carbonyl]-2,3,4,5- tetrahydro-1,4-benzoxazepin-7- yl}pyridin-2-amine |
| 491 | 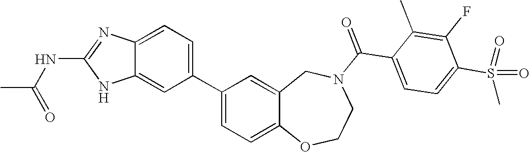 | N-[6-(4-{[3-fluoro-2-methyl-4- (methylsulfonyl)phenyl]carbonyl}- 2,3,4,5-tetrahydro-1,4- benzoxazepin-7-yl)-1H- benzimidazol-2-yl]acetamide |
| 492 | 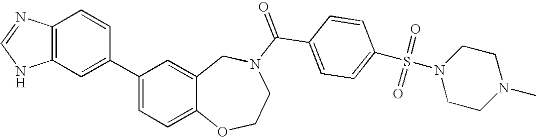 | 7-(1H-benzimidazol-6-yl)-4-({4- [(4-methylpiperazin-1- yl)sulfonyl]phenyl}carbonyl)- 2,3,4,5-tetrahydro-1,4- benzoxazepine |
| 493 | 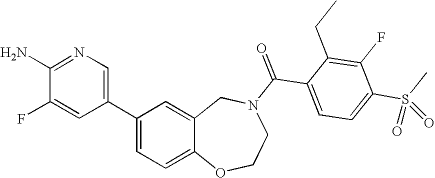 | 5-(4-{[2-ethyl-3-fluoro-4- (methylsulfonyl)phenyl]carbonyl}- 2,3,4,5-tetrahydro-1,4- benzoxazepin-7-yl)-3- fluoropyridin-2-amine |
| 494 | 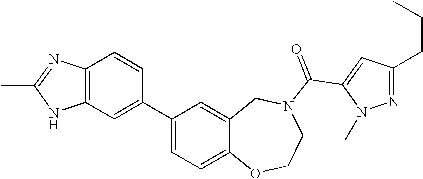 | 7-(2-methyl-1H-benzimidazol-6- yl)-4-[(1-methyl-3-propyl-1H- pyrazol-5-yl)carbonyl]-2,3,4,5- tetrahydro-1,4-benzoxazepine |
| 495 | 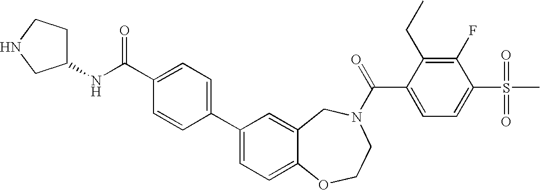 | 4-(4-{[2-ethyl-3-fluoro-4- (methylsulfonyl)phenyl]carbonyl}- 2,3,4,5-tetrahydro-1,4- benzoxazepin-7-yl)-N-[(3S)- pyrrolidin-3-yl]benzamide |
| 496 | 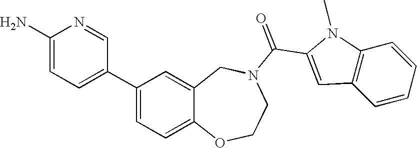 | 5-{4-[(1-methyl-1H-indol-2- yl)carbonyl]-2,3,4,5-tetrahydro- 1,4-benzoxazepin-7-yl}pyridin- 2-amine |
| 497 | 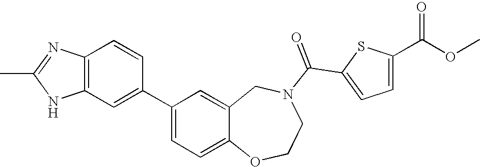 | methyl 5-{[7-(2-methyl-1H- benzimidazol-6-yl)-2,3-dihydro- 1,4-benzoxazepin-4(5H)- yl]carbonyl}thiophene-2- carboxylate |
| 498 | 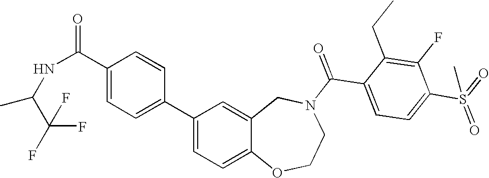 | 4-(4-{[2-ethyl-3-fluoro-4- (methylsulfonyl)phenyl]carbonyl}- 2,3,4,5-tetrahydro-1,4- benzoxazepin-7-yl)-N-(2,2,2- trifluoro-1- methylethyl)benzamide |
| 499 | 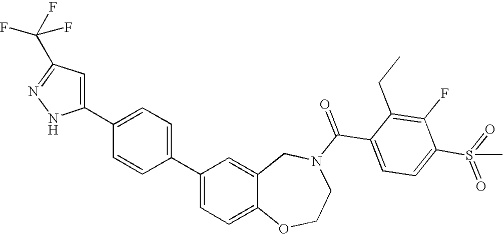 | 4-{[2-ethyl-3-fluoro-4- (methylsulfonyl)phenyl]carbonyl}- 7-(4-[3-(trifluoromethyl)-1H- pyrazol-5-yl]phenyl}-2,3,4,5- tetrahydro-1,4-benzoxazepine |
| 500 | 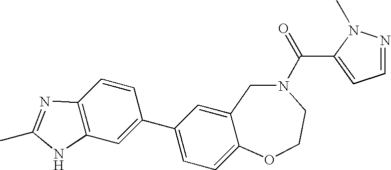 | 7-(2-methyl-1H-benzimidazol-6- yl)-4-[(1-methyl-1H-pyrazol-5- yl)carbonyl]-2,3,4,5-tetrahydro- 1,4-benzoxazepine |
| 501 | 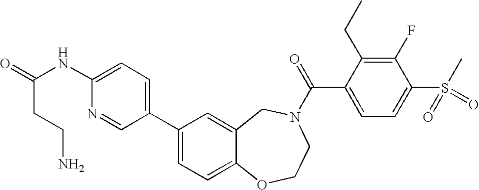 | N-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- beta-alaninamide |
| 502 | 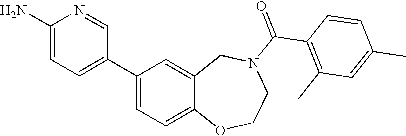 | 5-{4-[(2,4- dimethylphenyl)carbonyl]- 2,3,4,5-tetrahydro-1,4- benzoxazepin-7-yl}pyridin-2- amine |
| 503 | 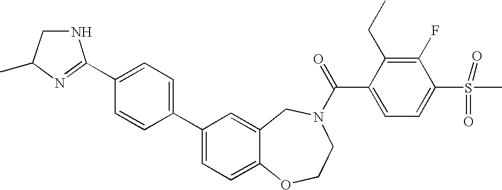 | 4-{[2-ethyl-3-fluoro-4- (methylsulfonyl)phenyl]carbonyl}- 7-[4-(4-methyl-4,5-dihydro- 1H-imidazol-2-yl)phenyl]- 2,3,4,5-tetrahydro-1,4- benzoxazepine |
| 504 | 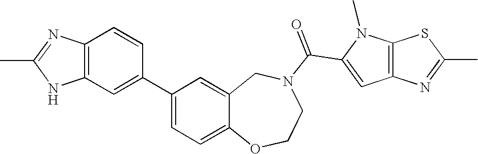 | 4-[(2,4-dimethyl-4H- pyrrolo[3,2-d][1,3]thiazol-5- yl)carbonyl]-7-(2-methyl-1H- benzimidazol-6-yl)-2,3,4,5- tetrahydro-1,4-benzoxazepine |
| 505 | 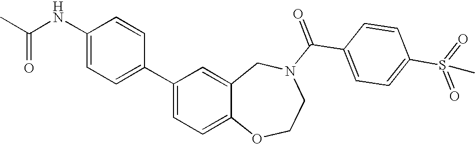 | N-[4-(4-{[4- (methylsulfonyl)phenyl]carbonyl}- 2,3,4,5-tetrahydro-1,4- benzoxazepin-7- yl)phenyl]acetamide |
| 506 | 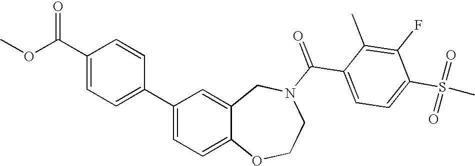 | methyl 4-(4-{[3-fluoro-2- methyl-4- (methylsulfonyl)phenyl]carbonyl}- 2,3,4,5-tetrahydro-1,4- benzoxazepin-7-yl)benzoate |
| 507 | 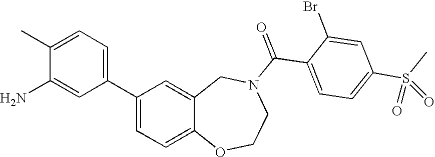 | 5-(4-{[2-bromo-4- (methylsulfonyl)phenyl]carbonyl}- 2,3,4,5-tetrahydro-1,4- benzoxazepin-7-yl)-2- methylaniline |
| 508 | 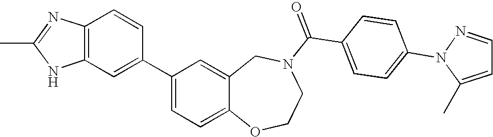 | 7-(2-methyl-1H-benzimidazol-6- yl)-4-{[4-(5-methyl-1H-pyrazol- 1-yl)phenyl]carbonyl}-2,3,4,5- tetrahydro-1,4-benzoxazepine |
| 509 | 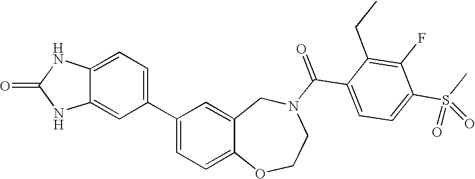 | 5-(4-{[2-ethyl-3-fluoro-4- (methylsulfonyl)phenyl]carbonyl}- 2,3,4,5-tetrahydro-1,4- benzoxazepin-7-yl)-1,3-dihydro- 2H-benzimidazol-2-one |
| 510 | 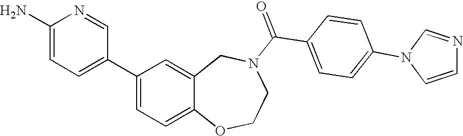 | 5-(4-{[4-(1H-imidazol-1- yl)phenyl]carbonyl}-2,3,4,5- tetrahydro-1,4-benzoxazepin-7- yl)pyridin-2-amine |
| 511 | 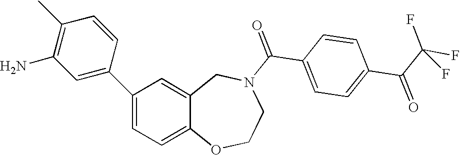 | l-(4-{[7-(3-amino-4- methylphenyl)-2,3-dihydro-1,4- benzoxazepin-4(5H)- yl]carbonyl}phenyl)-2,2,2- trifluoroethanone |
| 512 | 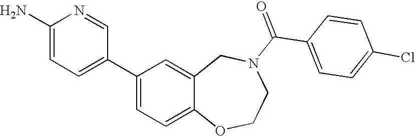 | 5-{4-[(4- chlorophenyl)carbonyl]-2,3,4,5- tetrahydro-1,4-benzoxazepin-7- yl}pyridin-2-amine |
| 513 | 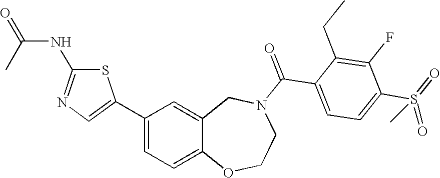 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]acetamide |
| 514 | 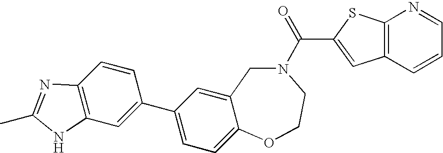 | 7-(2-methyl-1H-benzimidazol-6- yl)-4-(thieno[2,3-b]pyridin-2- ylcarbonyl)-2,3,4,5-tetrahydro- 1,4-benzoxazepine |
| 515 | 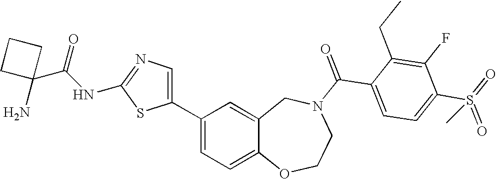 | 1-amino-N-[5-(4-{[2-ethyl-3- fluoro-4-(methylsulfonyl)phenyl] carbonyl}-2,3,4,5-tetrahydro-1,4- benzoxazepin-7-yl)-1,3-thiazol- 2-yl]cyclobutanecarboxamide |
| 516 | 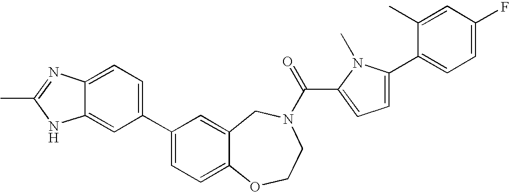 | 4-{[5-(4-fluoro-2- methylphenyl)-1-methyl-1H- pyrrol-2-yl]carbonyl}-7-(2- methyl-1H-benzimidazol-6-yl)- 2,3,4,5-tetrahydro-1,4- benzoxazepine |
| 517 | 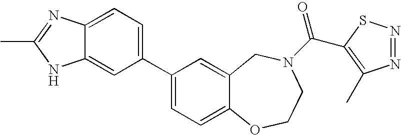 | 7-(2-methyl-1H-benzimidazol-6- yl)-4-[(4-methyl-1,2,3- thiadiazol-5-yl)carbonyl]- 2,3,4,5-tetrahydro-1,4- benzoxazepine |
| 518 | 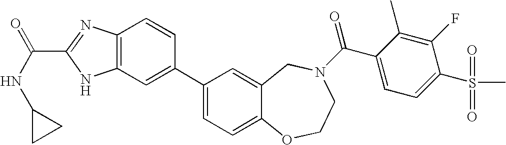 | N-cyclopropyl-6-(4-{[3-fluoro- 2-methyl-4- (methylsulfonyl)phenyl]carbonyl}- 2,3,4,5-tetrahydro-1,4- benzoxazepin-7-yl)-1H- benzimidazole-2-carboxamide |
| 519 | 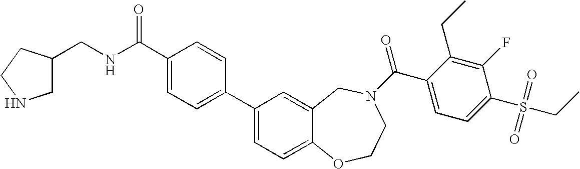 | 4-(4-{[2-ethyl-4-(ethylsulfonyl)- 3-fluorophenyl]carbonyl}- 2,3,4,5-tetrahydro-1,4- benzoxazepin-7-yl)-N- (pyrrolidin-3- ylmethyl)benzamide |
| 520 | 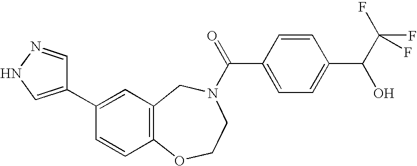 | 2,2,2-trifluoro-1-(4-{[7-(1H- pyrazol-4-yl)-2,3-dihydro-1,4- benzoxazepin-4(5H)- yl]carbonyl}phenyl)ethanol |
| 521 | 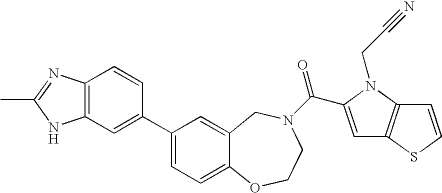 | (5-{[7-(2-methyl-1H- benzimidazol-6-yl)-2,3-dihydro- 1,4-benzoxazepin-4(5H)- yl]carbonyl}-4H-thieno[3,2- b]pyrrol-4-yl)acetonitrile |
| 522 | 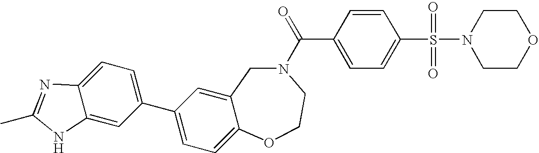 | 7-(2-methyl-1H-benzimidazol-6- yl)-4-{[4-(morpholin-4- ylsulfonyl)phenyl]carbonyl}- 2,3,4,5-tetrahydro-1,4- benzoxazepine |
| 523 | 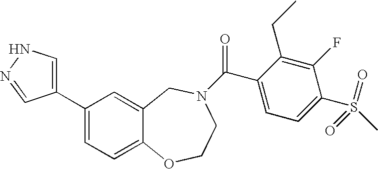 | 4-{[2-ethyl-3-fluoro-4- (methylsulfonyl)phenyl]carbonyl}- 7-(1H-pyrazol-4-yl)-2,3,4,5- tetrahydro-1,4-benzoxazepine |
| 524 | 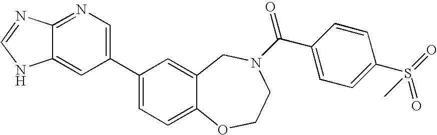 | 7-(1H-imidazo[4,5-b]pyridin-6- yl)-4-{[4- (methylsulfonyl)phenyl]carbonyl}- 2,3,4,5-tetrahydro-1,4- benzoxazepine |
| 525 | 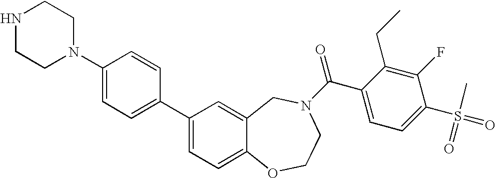 | 4-{[2-ethyl-3-fluoro-4- (methylsulfonyl)phenyl]carbonyl}- 7-(4-piperazin-1-ylphenyl)- 2,3,4,5-tetrahydro-1,4- benzoxazepine |
| 526 | 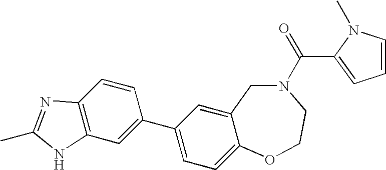 | 7-(2-methyl-1H-benzimidazol-6- yl)-4-[(1-methyl-1H-pyrrol-2- yl)carbonyl]-2,3,4,5-tetrahydro- 1,4-benzoxazepine |
| 527 | 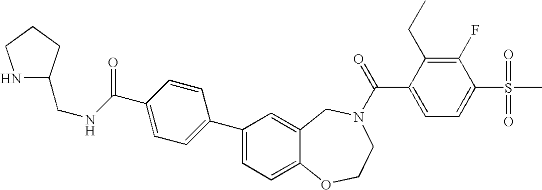 | 4-(4-{[2-ethyl-3-fluoro-4- (methylsulfonyl)phenyl]carbonyl}- 2,3,4,5-tetrahydro-1,4- benzoxazepin-7-yl)-N- (pyrrolidin-2- ylmethyl)benzamide |
| 528 | 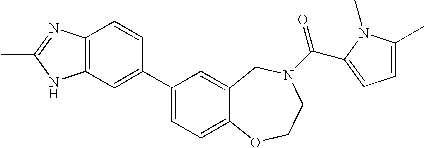 | 4-[(1,5-dimethyl-1H-pyrrol-2- yl)carbonyl]-7-(2-methyl-1H- benzimidazol-6-yl)-2,3,4,5- tetrahydro-1,4-benzoxazepine |
| 529 | 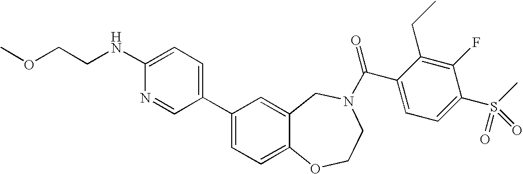 | 5-(4-{[2-ethyl-3-fluoro-4- (methylsulfonyl)phenyl]carbonyl}- 2,3,4,5-tetrahydro-1,4- benzoxazepin-7-yl)-N-[2- (methyloxy)ethyl]pyridin-2- amine |
| 530 | 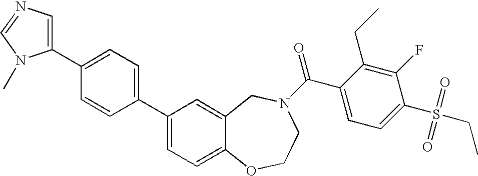 | 4-{[2-ethyl-4-(ethylsulfonyl)-3- fluorophenyl]carbonyl}-7-[4-(1- methyl-1H-imidazol-5- yl)phenyl]-2,3,4,5-tetrahydro- 1,4-benzoxazepine |
| 531 | 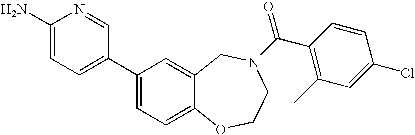 | 5-{4-[(4-chloro-2- methylphenyl)carbonyl]-2,3,4,5- tetrahydro-1,4-benzoxazepin-7- yl}pyridin-2-amine |
| 532 | 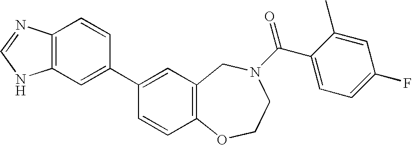 | 7-(1H-benzimidazol-6-yl)-4-[(4- fluoro-2- methylphenyl)carbonyl]-2,3,4,5- tetrahydro-1,4-benzoxazepine |
| 533 | 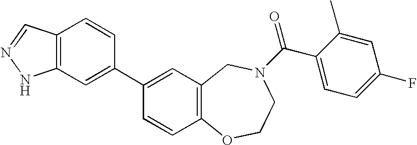 | 4-[(4-fluoro-2- methylphenyl)carbonyl]-7-(1H- indazol-6-yl)-2,3,4,5-tetrahydro- 1,4-benzoxazepine |
| 534 | 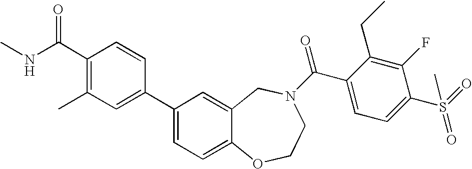 | 4-(4-{[2-ethyl-3-fluoro-4- (methylsulfonyl)phenyl]carbonyl}- 2,3,4,5-tetrahydro-1,4- benzoxazepin-7-yl)-N,2- dimethylbenzamide |
| 535 | 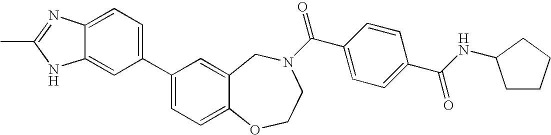 | N-cyclopentyl-4-{[7-(2-methyl- 1H-benzimidazol-6-yl)-2,3- dihydro-1,4-benzoxazepin- 4(5H)-yl]carbonyl}benzamide |
| 536 | 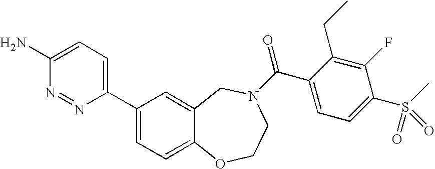 | 6-(4-{[2-ethyl-3-fluoro-4- (methylsulfonyl)phenyl]carbonyl}- 2,3,4,5-tetrahydro-1,4- benzoxazepin-7-yl)pyridazin-3- amine |
| 537 | 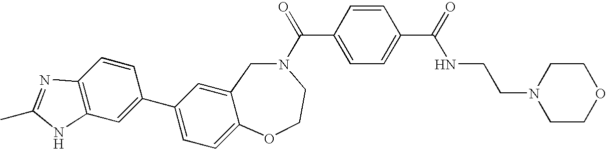 | 4-{[7-(2-methyl-1H- benzimidazol-6-yl)-2,3-dihydro- 1,4-benzoxazepin-4(5H)- yl]carbonyl}-N-(2-morpholin-4- ylethyl)benzamide |
| 538 | 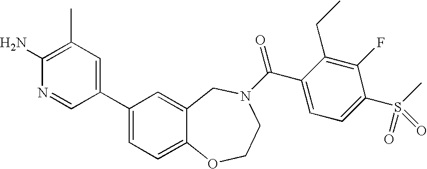 | 5-(4-{[2-ethyl-3-fluoro-4- (methylsulfonyl)phenyl]carbonyl}- 2,3,4,5-tetrahydro-1,4- benzoxazepin-7-yl)-3- methylpyridin-2-amine |
| 539 | 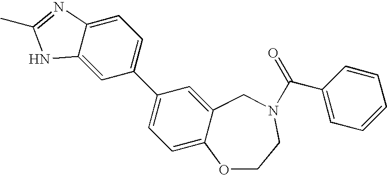 | 7-(2-methyl-1H-benzimidazol-6- yl)-4-(phenylcarbonyl)-2,3,4,5- tetrahydro-1,4-benzoxazepine |
| 540 | 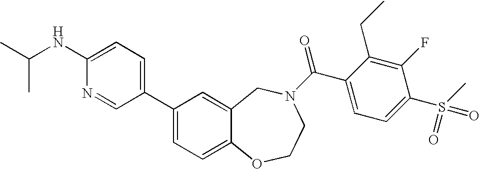 | 5-(4-{[2-ethyl-3-fluoro-4- (methylsulfonyl)phenyl]carbonyl}- 2,3,4,5-tetrahydro-1,4- benzoxazepin-7-yl)-N-(1- methylethyl)pyridin-2-amine |
| 541 | 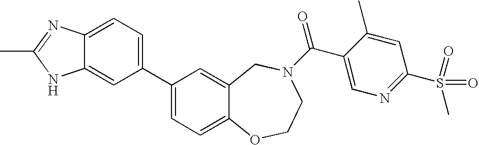 | 7-(2-methyl-1H-benzimidazol-6- yl)-4-{[4-methyl-6- (methylsulfonyl)pyridin-3- yl]carbonyl}-2,3,4,5-tetrahydro- 1,4-benzoxazepine |
| 542 | 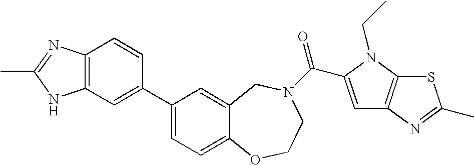 | 4-[(4-ethyl-2-methyl-4H- pyrrolo[3,2-d][1,3]thiazol-5- yl)carbonyl]-7-(2-methyl-1H- benzimidazol-6-yl)-2,3,4,5- tetrahydro-1,4-benzoxazepine |
| 543 | 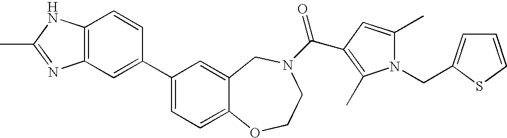 | 4-{[2,5-dimethyl-1-(2- thienylmethyl)-1H-pyrrol-3- yl]carbonyl}-7-(2-methyl-1H- benzimidazol-5-yl)-2,3,4,5- tetrahydro-1,4-benzoxazepine |
| 544 | 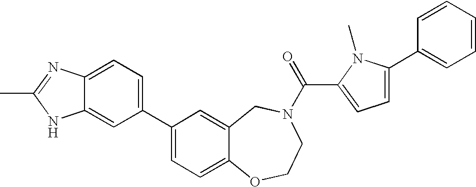 | 7-(2-methyl-1H-benzimidazol-6- yl)-4-[(1-methyl-5-phenyl-1H- pyrrol-2-yl)carbonyl]-2,3,4,5- tetrahydro-1,4-benzoxazepine |
| 545 | 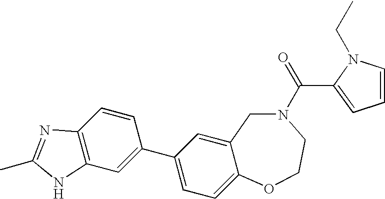 | 4-[(1-ethyl-1H-pyrrol-2- yl)carbonyl]-7-(2-methyl-1H- benzimidazol-6-yl)-2,3,4,5- tetrahydro-1,4-benzoxazepine |
| 546 | 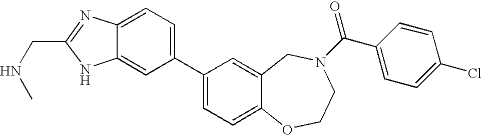 | l-(6-{4-[(4- chlorophenyl)carbonyl]-2,3,4,5- tetrahydro-1,4-benzoxazepin-7- yl}-1H-benzimidazol-2-yl)-N- methylmethanamine |
| 547 | 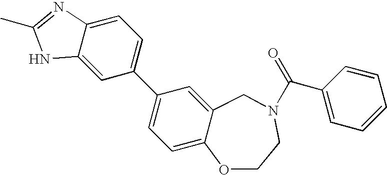 | 7-(1H-benzimidazol-6-yl)-4-{[2- chloro-4- (methylsulfonyl)phenyl]carbonyl}- 2,3,4,5-tetrahydro-1,4- benzoxazepine |
| 548 | 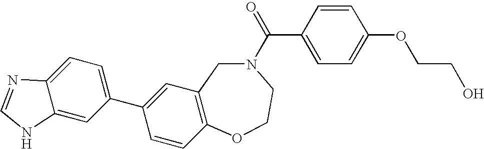 | 2-[(4-{[7-(1H-benzimidazol-6- yl)-2,3-dihydro-1,4- benzoxazepin-4(5H)- yl]carbonyl}phenyl)oxy]ethanol |
| 549 | 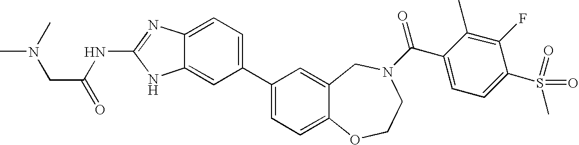 | N-[6-(4-{[3-fluoro-2-methyl-4- (methylsulfonyl)phenyl]carbonyl}- 2,3,4,5-tetrahydro-1,4- benzoxazepin-7-yl)-1H- benzimidazol-2-yl]-N²,N²- dimethylglycinamide |
| 550 | 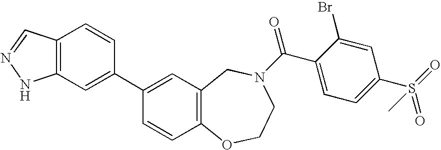 | 4-{[2-bromo-4- (methylsulfonyl)phenyl]carbonyl}- 7-(1H-indazol-6-yl)-2,3,4,5- tetrahydro-1,4-benzoxazepine |
| 551 | 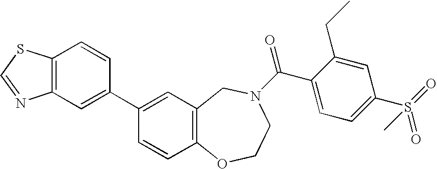 | 7-(1,3-benzothiazol-5-yl)-4-{[2- ethyl-4- (mcthylsulfonyl)phenyl]carbonyl}- 2,3,4,5-tetrahydro-1,4- benzoxazepine |
| 552 | 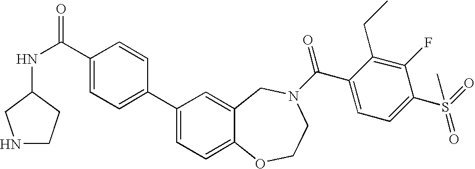 | 4-(4-{[2-ethyl-3-fluoro-4- (methylsulfonyl)phenyl]carbonyl}- 2,3,4,5-tetrahydro-1,4- benzoxazepin-7-yl)-N- pyrrolidin-3-ylbenzamide |
| 553 | 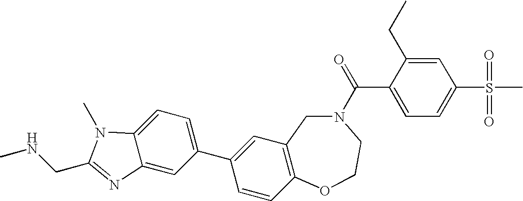 | l-[5-(4-{[2-ethyl-4- (methylsulfonyl)phenyl]carbonyl}- 2,3,4,5-tetrahydro-1,4- benzoxazepin-7-yl)-1-methyl- 1H-benzimidazol-2-yl]-N- methylmethanamine |
| 554 | 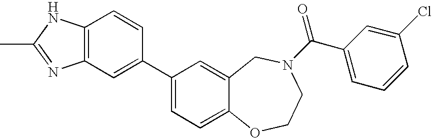 | 4-[(3-chlorophenyl)carbonyl]-7- (2-methyl-1H-benzimidazol-5- yl)-2,3,4,5-tetrahydro-1,4- benzoxazepine |
| 555 | 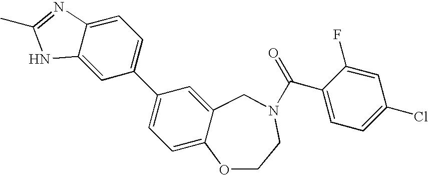 | 4-[(4-chloro-2- fiuorophenyl)carbonyl]-7-(2- methyl-1H-benzimidazol-6-yl)- 2,3,4,5-tetrahydro-1,4- benzoxazepine |
| 556 | 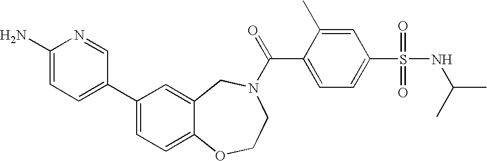 | 4-{[7-(6-aminopyridin-3-yl)-2,3- dihydro-1,4-benzoxazepin- 4(5H)-yl]carbonyl}-3-methyl-N-(1- methylethyl)benzenesulfonamide |
| 557 | 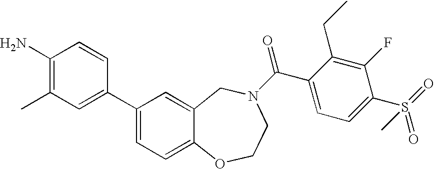 | 4-(4-{[2-ethyl-3-fluoro-4- (methylsulfonyl)phenyl]carbonyl}- 2,3,4,5-tetrahydro-1,4- benzoxazepin-7-yl)-2- methylaniline |
| 558 | 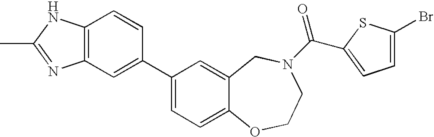 | 4-[(5-bromo-2- thienyl)carbonyl]-7-(2-methyl- 1H-benzimidazol-5-yl)-2,3,4,5- tetrahydro-1,4-benzoxazepine |
| 559 | 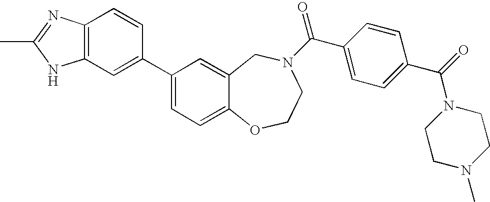 | 7-(2-methyl-1H-benzimidazol-6- yl)-4-({4-[(4-methylpiperazin-1- yl)carbonyl]phenyl}carbonyl)- 2,3,4,5-tetrahydro-1,4- benzoxazepine |
| 560 | 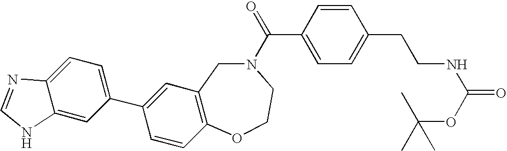 | 1,1-dimethylethyl[2-(4-{[7-(1H- benzimidazol-6-yl)-2,3-dihydro- 1,4-benzoxazepin-4(5H)- yl]carbonyl}phenyl)ethyl]carbamate |
| 561 | 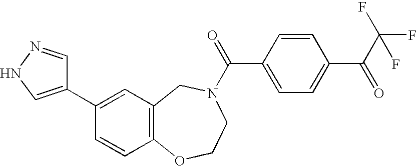 | 2,2,2-trifluoro-1-(4-{[7-(1H- pyrazol-4-yl)-2,3-dihydro-1,4- benzoxazepin-4(5H)- yl]carbonyl}phenyl)ethanone |
| 562 | 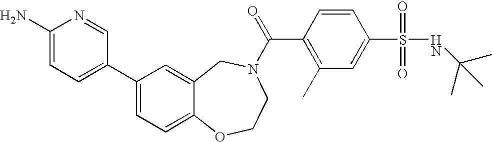 | 4-{[7-(6-aminopyridin-3-yl)-2,3- dihydro-1,4-benzoxazepin- 4(5H)-yl]carbonyl}-N-(1,1- dimethylethyl)-3- methylbenzenesulfonamide |
| 563 | 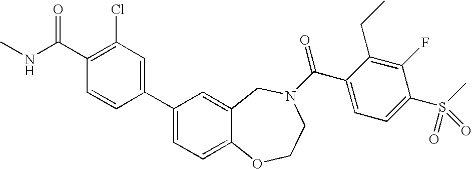 | 2-chloro-4-(4-{[2-ethyl-3-fluoro-4- (methylsulfonyl)phenyl]carbonyl}- 2,3,4,5-tetrahydro-1,4- benzoxazepin-7-yl)-N- methylbenzamide |
| 564 | 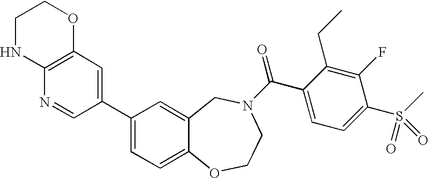 | 7-(3,4-dihydro-2H-pyrido[3,2-b] [1,4]oxazin-7-yl)-4-{[2-ethyl- 3-fluoro-4- (methylsulfonyl)phenyl]carbonyl}- 2,3,4,5-tetrahydro-1,4- benzoxazepine |
| 565 | 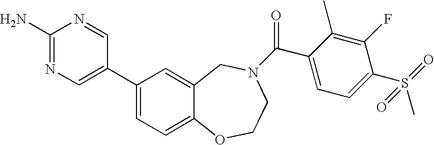 | 5-(4-{[3-fluoro-2-methyl-4- (methylsulfonyl)phenyl]carbonyl}- 2,3,4,5-tetrahydro-1,4- benzoxazepin-7-yl)pyrimidin-2- amine |
| 566 | 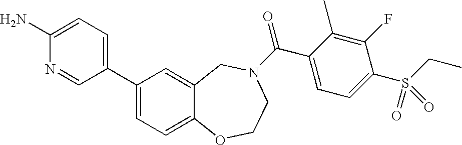 | 5-(4-{[4-(ethylsulfonyl)-3- fluoro-2- methylphenyl]carbonyl}-2,3,4,5- tetrahydro-1,4-benzoxazepin-7- yl)pyridin-2-amine |
| 567 | 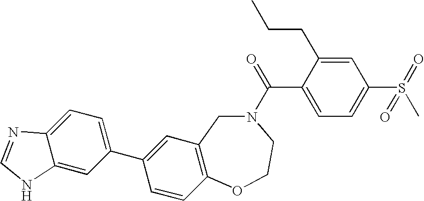 | 7-(1H-benzimidazol-6-yl)-4-{[4- (methylsulfonyl)-2- propylphenyl]carbonyl}-2,3,4,5- tetrahydro-1,4-benzoxazepine |
| 568 | 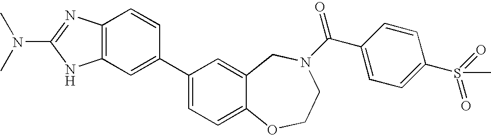 | N,N-dimethyl-6-(4-{[4- (methylsulfonyl)phenyl]carbonyl}- 2,3,4,5-tetrahydro-1,4- benzoxazepin-7-yl)-1H- benzimidazol-2-amine |
| 569 | 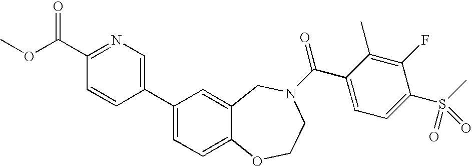 | methyl 5-(4-{[3-fluoro-2- methyl-4- (methylsulfonyl)phenyl]carbonyl}- 2,3,4,5-tetrahydro-1,4- benzoxazepin-7-yl)pyridine-2- carboxylate |
| 570 | 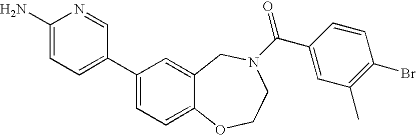 | 5-{4-[(4-bromo-3- methylphenyl)carbonyl]-2,3,4,5- tetrahydro-1,4-benzoxazepin-7- yl}pyridin-2-amine |
| 571 | 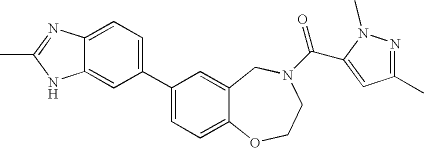 | 4-[(1,3-dimethyl-1H-pyrazol-5- yl)carbonyl]-7-(2-methyl-1H- benzimidazol-6-yl)-2,3,4,5- tetrahydro-1,4-benzoxazepine |
| 572 | 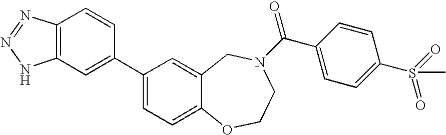 | 7-(1H-1,2,3-benzotriazol-6-yl)- 4-{[4- (methylsulfonyl)phenyl]carbonyl}- 2,3,4,5-tetrahydro-1,4- benzoxazepine |
| 573 | 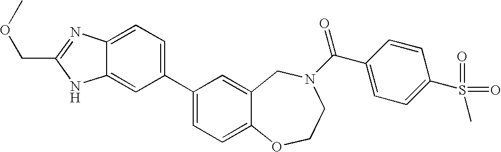 | 7-{2-[(methyloxy)methyl]-1H- benzimidazol-6-yl}-4-{[4- (methylsulfonyl)phenyl]carbonyl}- 2,3,4,5-tetrahydro-1,4- benzoxazepine |
| 574 | 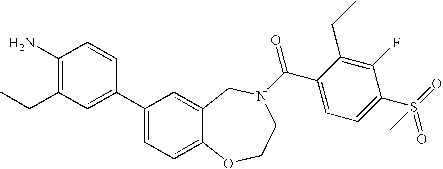 | 2-ethyl-4-(4-{[2-ethyl-3-fluoro-4- (methylsulfonyl)phenyl]carbonyl}- 2,3,4,5-tetrahydro-1,4- benzoxazepin-7-yl)aniline |
| 575 | 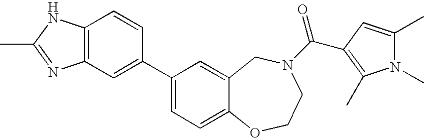 | 7-(2-methyl-1H-benzimidazol-5- yl)-4-[(1,2,5-trimethyl-1H- pyrrol-3-yl)carbonyl]-2,3,4,5- tetrahydro-1,4-benzoxazepine |
| 576 | 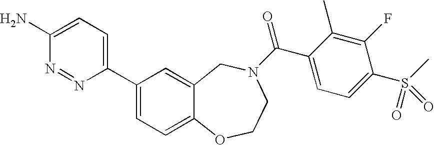 | 6-(4-{[3-fluoro-2-methyl-4- (methylsulfonyl)phenyl]carbonyl}- 2,3,4,5-tetrahydro-1,4- benzoxazepin-7-yl)pyridazin-3- amine |
| 577 | 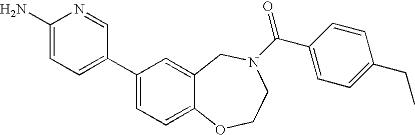 | 5-{4-[(4-ethylphenyl)carbonyl]- 2,3,4,5-tetrahydro-1,4- benzoxazepin-7-yl}pyridin-2- amine |
| 578 | 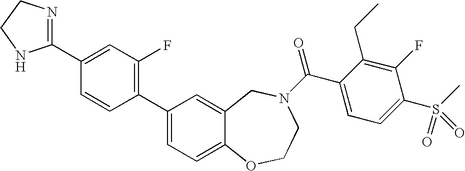 | 7-[4-(4,5-dihydro-1H-imidazol- 2-yl)-2-fluorophenyl]-4-{[2- ethyl-3-fluoro-4- (methylsulfonyl)phenyl]carbonyl}- 2,3,4,5-tetrahydro-1,4- benzoxazepine |
| 579 | 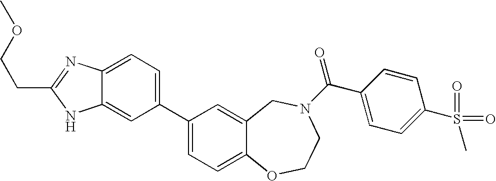 | 7-{2-[2-(methyloxy)ethyl]-1H- benzimidazol-6-yl}-4-{[4- (methylsulfonyl)phenyl]carbonyl}- 2,3,4,5-tetrahydro-1,4- benzoxazepine |
| 580 | 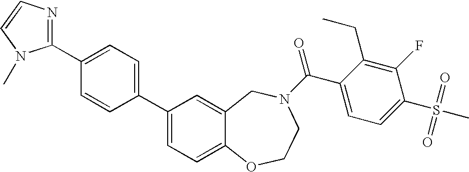 | 4-{[2-ethyl-3-fluoro-4- (methylsulfonyl)phenyl]carbonyl}- 7-[4-(1-methyl-1H-imidazol-2- yl)phenyl]-2,3,4,5-tetrahydro- 1,4-benzoxazepine |
| 581 | 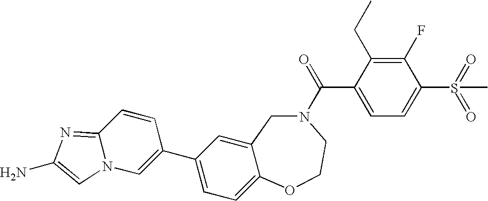 | 6-(4-{[2-ethyl-3-fluoro-4- (methylsulfonyl)phenyl]carbonyl}- 2,3,4,5-tetrahydro-1,4- benzoxazepin-7-yl)imidazo[1,2- a]pyridin-2-amine |
| 582 | 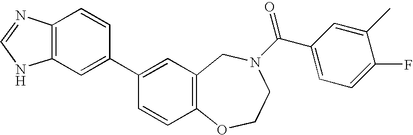 | 7-(1H-benzimidazol-6-yl)-4-[(4- fluoro-3- methylphenyl)carbonyl]-2,3,4,5- tetrahydro-1,4-benzoxazepine |
| 583 | 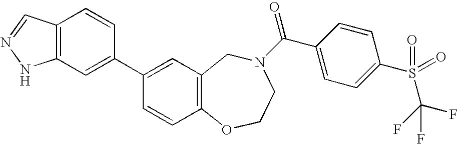 | 7-(1H-indazol-6-yl)-4-({4- [(trifluoromethyl)sulfonyl]phenyl}- carbonyl)-2,3,4,5-tetrahydro- 1,4-benzoxazepine |
| 584 | 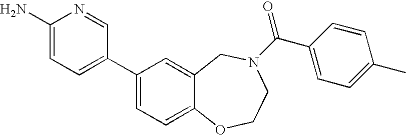 | 5-{4-[(4- methylphenyl)carbonyl]-2,3,4,5- tetrahydro-1,4-benzoxazepin-7- yl}pyridin-2-amine |
| 585 | 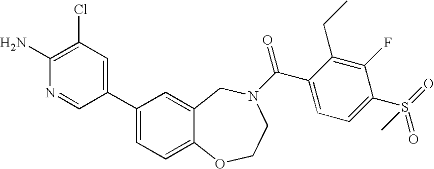 | 3-chloro-5-(4-{[2-ethyl-3-fluoro-4- (methylsulfonyl)phenyl]carbonyl}- 2,3,4,5-tetrahydro-1,4- benzoxazepin-7-yl)pyridin-2- amine |
| 586 | 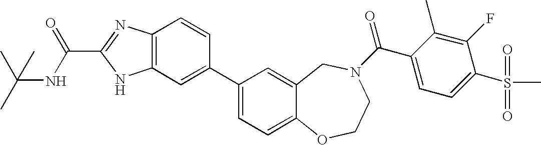 | N-(1,1-dimethylethyl)-6-(4-{[3- fluoro-2-methyl-4- (methylsulfonyl)phenyl]carbonyl}- 2,3,4,5-tetrahydro-1,4- benzoxazepin-7-yl)-1H- benzimidazole-2-carboxamide |
| 587 | 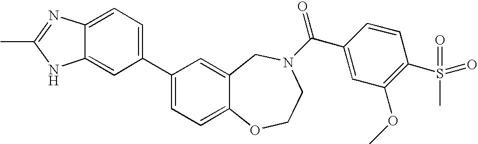 | 7-(2-methyl-1H-benzimidazol-6- yl)-4-{[3-(methyloxy)-4- (methylsulfonyl)phenyl]carbonyl}- 2,3,4,5-tetrahydro-1,4- benzoxazepine |
| 588 | 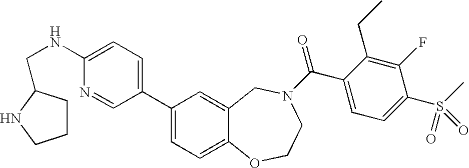 | 5-(4-{[2-ethyl-3-fluoro-4- (methylsulfonyl)phenyl]carbonyl}- 2,3,4,5-tetrahydro-1,4- benzoxazepin-7-yl)-N- (pyrrolidin-2-ylmethyl)pyridin- 2-amine |
| 589 | 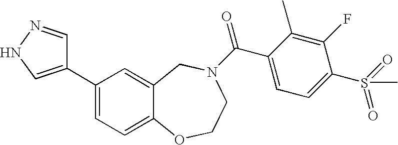 | 4-{[3-fluoro-2-methyl-4- (methylsulfonyl)phenyl]carbonyl}- 7-(1H-pyrazol-4-yl)-2,3,4,5- tetrahydro-1,4-benzoxazepine |
| 590 | 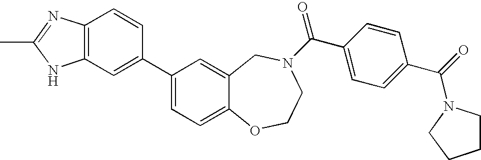 | 7-(2-methyl-1H-benzimidazol-6- yl)-4-{[4-(pyrrolidin-1- ylcarbonyl)phenyl]carbonyl}- 2,3,4,5-tetrahydro-1,4- benzoxazepine |
| 591 | 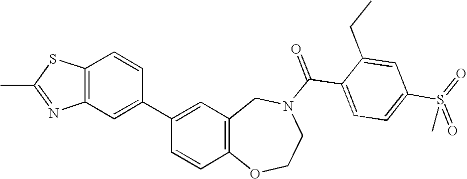 | 4-{[2-ethyl-4- (methylsulfonyl)phenyl]carbonyl}- 7-(2-methyl-1,3-benzothiazol- 5-yl)-2,3,4,5-tetrahydro-1,4- benzoxazepine |
| 592 | 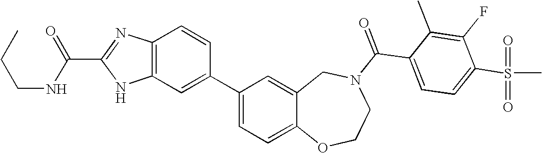 | 6-(4-{[3-fluoro-2-methyl-4- (methylsulfonyl)phenyl]carbonyl}- 2,3,4,5-tetrahydro-1,4- benzoxazepin-7-yl)-N-propyl- 1H-benzimidazole-2- carboxamide |
| 593 | 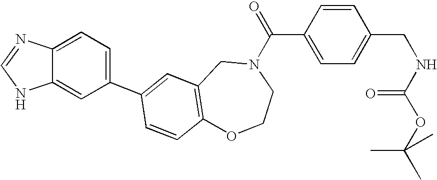 | 1,1-dimethylethyl[(4-{[7-(1H- benzimidazol-6-yl)-2,3-dihydro- 1,4-benzoxazepin-4(5H)- yl]carbonyl}phenyl)methyl] carbamate |
| 594 | 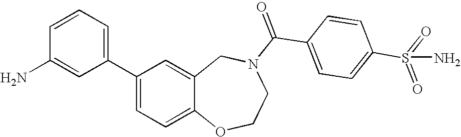 | 4-{[7-(3-aminophenyl)-2,3- dihydro-1,4-benzoxazepin- 4(5H)- yl]carbonyl}benzenesulfonamide |
| 595 | 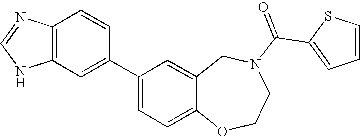 | 7-(1H-benzimidazol-6-yl)-4-(2- thienylcarbonyl)-2,3,4,5- tetrahydro-1,4-benzoxazepine |
| 596 | 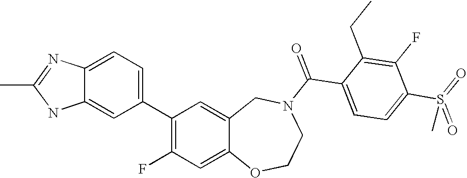 | 4-{[2-ethyl-3-fluoro-4- (methylsulfonyl)phenyl]carbonyl}- 8-fluoro-7-(2-methyl-1H- benzimidazol-6-yl)-2,3,4,5- tetrahydro-1,4-benzoxazepine |
| 597 | 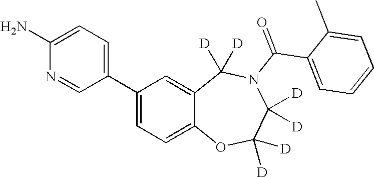 | [7-(6-aminopyridin-3- yl)(2,2,3,3,5,5-²H₆)-2,3-dihydro- 1,4-benzoxazepin-4(5H)-yl][2- methylL-phenyl]methanone |
| 598 | 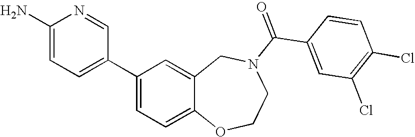 | 5-{4-[(3,4- dichlorophenyl)carbonyl]- 2,3,4,5-tetrahydro-1,4- benzoxazepin-7-yl}pyridine-2- amine |
| 599 | 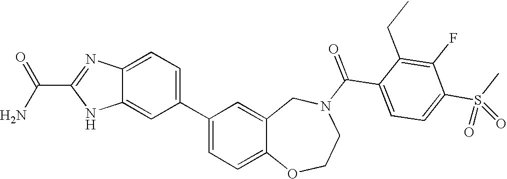 | 6-(4-{[2-ethyl-3-fluoro-4- (methylsulfonyl)phenyl]carbonyl}- 2,3,4,5-tetrahydro-1,4- benzoxazepin-7-yl)-1H- benzimidazole-2-carboxamide |
| 600 | 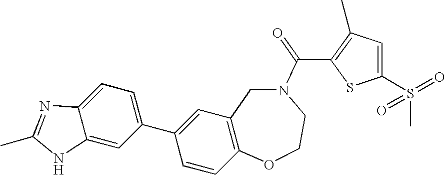 | 7-(2-methyl-1H-benzimidazol-6- yl)-4-{[3-methyl-5- (methylsulfonyl)-2- thienyl]carbonyl}-2,3,4,5- tetrahydro-1,4-benzoxazepine |
| 601 | 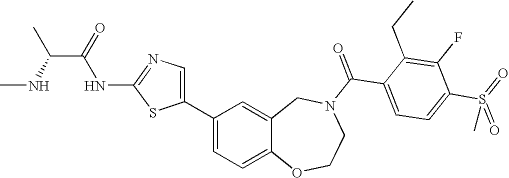 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]-N²-methyl-D-alaninamide |
| 602 | 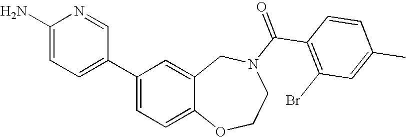 | 5-{4-[(2-bromo-4- methylphenyl)carbonyl]-2,3,4,5- tetrahydro-1,4-benzoxazepin-7- yl}pyridin-2-amine |
| 603 | 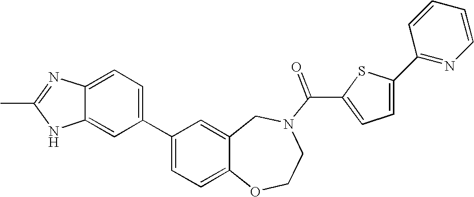 | 7-(2-methyl-1H-benzimidazol-6- yl)-4-[(5-pyridin-2-yl-2- thienyl)carbonyl]-2,3,4,5- tetrahydro-1,4-benzoxazepine |
| 604 | 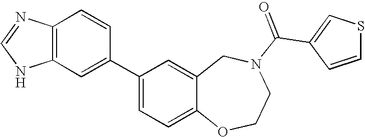 | 7-(1H-benzimidazol-6-yl)-4-(3- thienylcarbonyl)-2,3,4,5- tetrahydro-1,4-benzoxazepine |
| 605 | 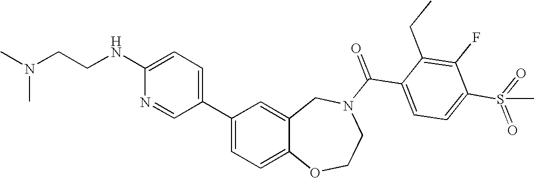 | N′-[5-(4-{[2-ethyl-3-fluoro-4- (mcthylsulfonyl)phenyl]carbonyl}- 2,3,4,5-tetrahydro-1,4- benzoxazepin-7-yl)pyridin-2-yl]- N,N-dimethylethane-1,2-diamine |
| 606 | 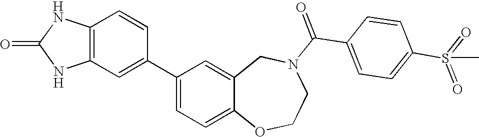 | 5-(4-{[4- (methylsulfonyl)phenyl]carbonyl}- 2,3,4,5-tetrahydro-1,4- benzoxazepin-7-yl)-1,3-dihydro- 2H-benzimidazol-2-one |
| 607 | 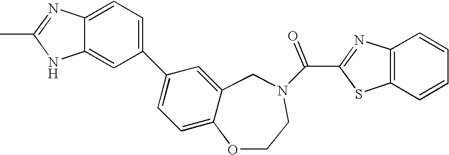 | 4-(1,3-benzothiazol-2- ylcarbonyl)-7-(2-methyl-1H- benzimidazol-6-yl)-2,3,4,5- tetrahydro-1,4-benzoxazepine |
| 608 | 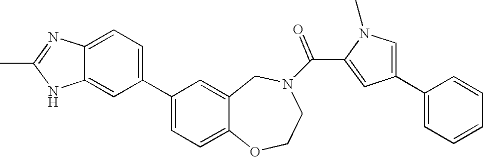 | 7-(2-methyl-1H-benzimidazol-6- yl)-4-[(1-methyl-4-phenyl-1H- pyrrol-2-yl)carbonyl]-2,3,4,5- tetrahydro-1,4-benzoxazepine |
| 609 | 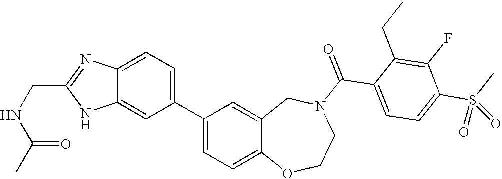 | N-{[6-(4-{[2-ethyl-3-fluoro-4- (methylsulfonyl)phenyl]carbonyl}- 2,3,4,5-tetrahydro-1,4- benzoxazepin-7-yl)-1H- benzimidazol-2- yl]methyl}acetamide |
| 610 | 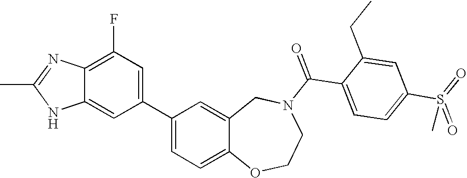 | 4-{[2-ethyl-4- (methylsulfonyl)phenyl]carbonyl}- 7-(4-fluoro-2-methyl-1H- benzimidazol-6-yl)-2,3,4,5- tetrahydro-1,4-benzoxazepine |
| 611 | 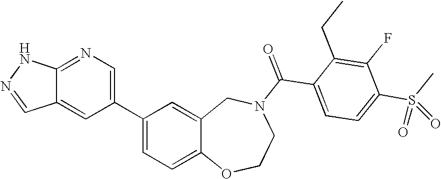 | 4-{[2-ethyl-3-fluoro-4- (methylsulfonyl)phenyl]carbonyl}- 7-(1H-pyrazolo[3,4-b]pyridin- 5-yl)-2,3,4,5-tetrahydro-1,4- benzoxazepine |
| 612 | 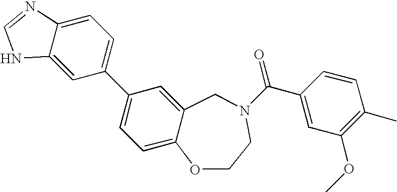 | 7-(1H-benzimidazol-6-yl)-4-{[4- methyl-3- (methyloxy)phenyl]carbonyl}- 2,3,4,5-tetrahydro-1,4- benzoxazepine |
| 613 | 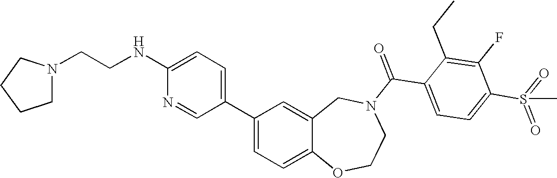 | 5-(4-{[2-ethyl-3-fluoro-4- (methylsulfonyl)phenyl]carbonyl}- 2,3,4,5-tetrahydro-1,4- benzoxazepin-7-yl)-N-(2- pyrrolidin-1-ylethyl)pyridin-2- amine |
| 614 | 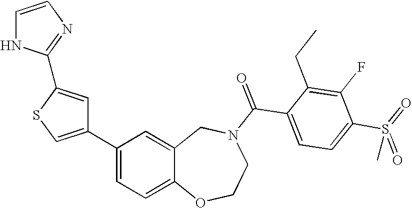 | 4-{[2-ethyl-3-fluoro-4- (methylsulfonyl)phenyl]carbonyl}- 7-[5-(1H-imidazol-2-yl)-3- thienyl]-2,3,4,5-tetrahydro-1,4- benzoxazepine |
| 615 | 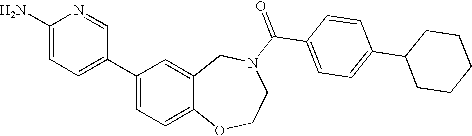 | 5-{4-[(4- cyclohexylphenyl)carbonyl]- 2,3,4,5-tetrahydro-1,4- benzoxazepin-7-yl}pyridin-2- amine |
| 616 | 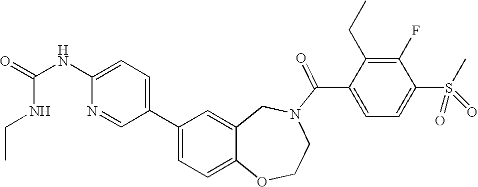 | 1-ethyl-3-[5-(4-{[2-ethyl-3- fluoro-4- (methylsulfonyl)phenyl]carbonyl}- 2,3,4,5-tetrahydro-1,4- benzoxazepin-7-yl)pyridin-2- yl]urea |
| 617 | 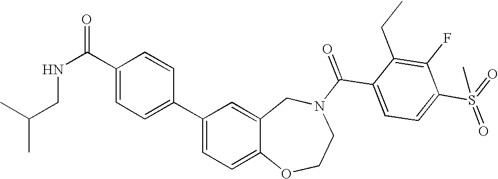 | 4-(4-{[2-ethyl-3-fluoro-4- (methylsulfonyl)phenyl]carbonyl}- 2,3,4,5-tetrahydro-1,4- benzoxazepin-7-yl)-N-(2- methylpropyl)benzamide |
| 618 | 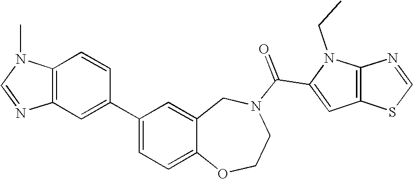 | 4-[(4-ethyl-4H-pyrrolo[2,3- d][1,3]thiazol-5-yl)carbonyl]-7- (1-methyl-1H-benzimidazol-5- yl)-2,3,4,5-tetrahydro-1,4- benzoxazepine |
| 619 | 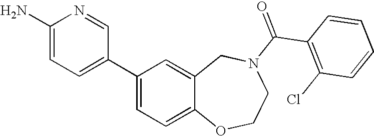 | 5-{4-[(2- chlorophenyl)carbonyl]-2,3,4,5- tetrahydro-1,4-benzoxazepin-7- yl}pyridin-2-amine |
| 620 | 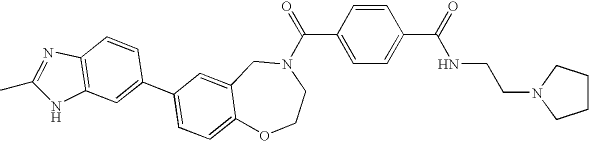 | 4-{[7-(2-methyl-1H- benzimidazol-6-yl)-2,3-dihydro- 1,4-benzoxazepin-4(5H)- yl]carbonyl}-N-(2-pyrrolidin-1- ylethyl)benzamide |
| 621 | 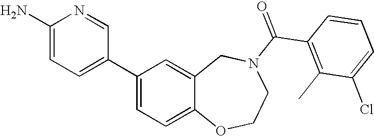 | 5-{4-[(3-chloro-2- methylphenyl)carbonyl]-2,3,4,5- tetrahydro-1,4-benzoxazepin-7- yl}pyridin-2-amine |
| 622 | 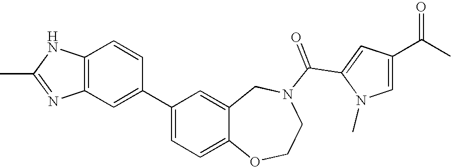 | 1-(1-methyl-5-{[7-(2-methyl- 1H-benzimidazol-5-yl)-2,3- dihydro-1,4-benzoxazepin- 4(5H)-yl]carbonyl}-1H-pyrrol-3- yl)ethanone |
| 623 | 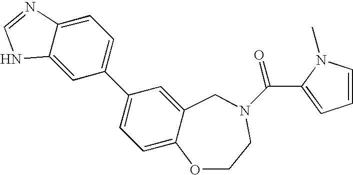 | 7-(1H-benzimidazol-6-yl)-4-[(1- methyl-1H-pyrrol-2- yl)carbonyl]-2,3,4,5-tetrahydro- 1,4-benzoxazepine |
| 624 | 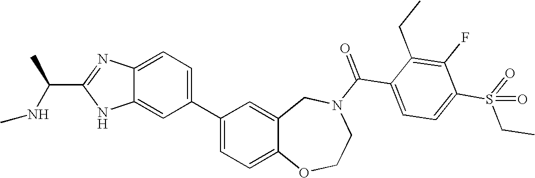 | (1S)-1-[6-(4-{[2-ethyl-4- (ethylsulfonyl)-3- fluorophenyl]carbonyl}-2,3,4,5- tetrahydro-1,4-benzoxazepin-7- yl)-1H-benzimidazol-2-yl]-N- methylethanamine |
| 625 | 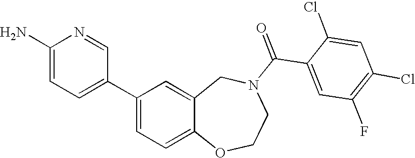 | 5-{4-[(2,4-dichloro-5- fluorophenyl)carbonyl]-2,3,4,5- tetrahydro-1,4-benzoxazepin-7- yl}pyridin-2-amine |
| 626 | 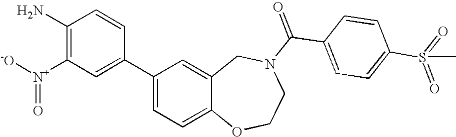 | 4-(4-{[4- (methylsulfonyl)phenyl]carbonyl}- 2,3,4,5-tetrahydro-1,4- benzoxazepin-7-yl)-2- nitroaniline |
| 627 | 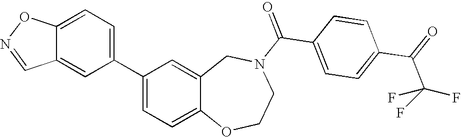 | 1-(4-{[7-(1,2-benzisoxazol-5- yl)-2,3-dihydro-1,4- benzoxazepin-4(5H)- yl]carbonyl}phenyl)-2,2,2- trifluoroethanone |
| 628 | 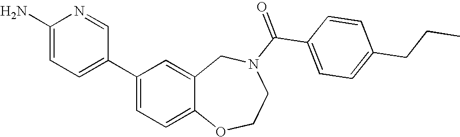 | 5-{4-[(4- propylphenyl)carbonyl]-2,3,4,5- tetrahydro-1,4-benzoxazepin-7- yl}pyridin-2-amine |
| 629 | 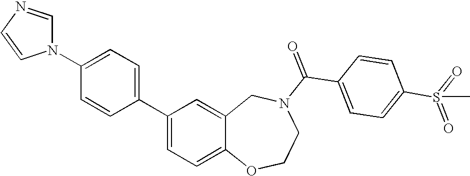 | 7-[4-(1H-imidazol-1-yl)phenyl]- 4-{[4- (methylsulfonyl)phenyl]carbonyl}- 2,3,4,5-tetrahydro-1,4- benzoxazepine |
| 630 | 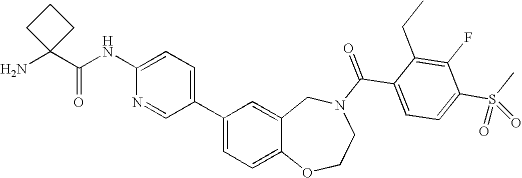 | 1-amino-N-[5-(4-{[2-ethyl-3- fluoro-4- (methylsulfonyl)phenyl]carbonyl}- 2,3,4,5-tetrahydro-1,4- benzoxazepin-7-yl)pyridin-2- yl]cyclobutanecarboxamide |
| 631 | 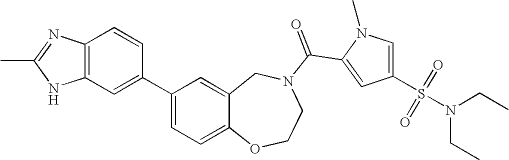 | N,N-diethyl-1-methyl-5-{[7-(2- methyl-1H-benzimidazol-6-yl)- 2,3-dihydro-1,4-benzoxazepin- 4(5H)-yl]carbonyl}-1H-pyrrole- 3-sulfonamide |
| 632 | 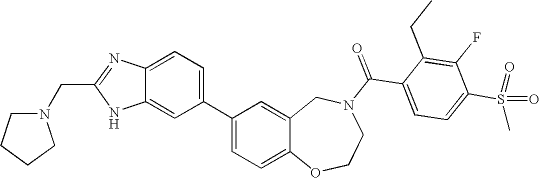 | 4-{[2-ethyl-3-fluoro-4- (methylsulfonyl)phenyl]carbonyl}- 7-[2-(pyrrolidin-1-ylmethyl)- 1H-benzimidazol-6-yl]-2,3,4,5- tetrahydro-1,4-benzoxazepine |
| 633 | 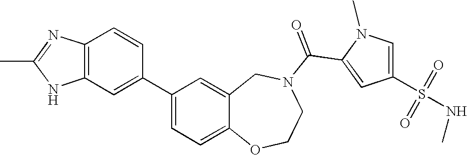 | N,1-dimethyl-5-{[7-(2-methyl- 1H-benzimidazol-6-yl)-2,3- dihydro-1,4-benzoxazepin- 4(5H)-yl]carbonyl}-1H-pyrrole- 3-sulfonamide |
| 634 | 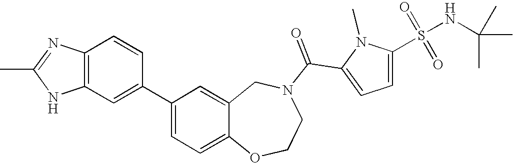 | N-(1,1-dimethylethyl)-1-methyl- 5-{[7-(2-methyl-1H- benzimidazol-6-yl)-2,3-dihydro- 1,4-benzoxazepin-4(5H)- yl]carbonyl}-1H-pyrrole-2- sulfonamide |
| 635 | 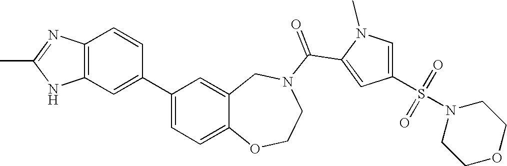 | 7-(2-methyl-1H-benzimidazol-6- yl)-4-{[1-methyl-4-(morpholin- 4-ylsulfonyl)-1H-pyrrol-2- yl]carbonyl}-2,3,4,5-tetrahydro- 1,4-benzoxazepine |
| 636 | 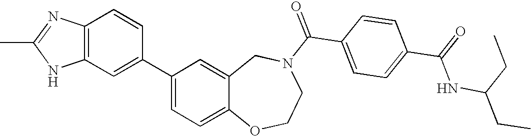 | N-(1-ethylpropyl)-4-{[7-(2- methyl-1H-benzimidazol-6-yl)- 2,3-dihydro-1,4-benzoxazepin- 4(5H)-yl]carbonyl}benzamide |
| 637 | 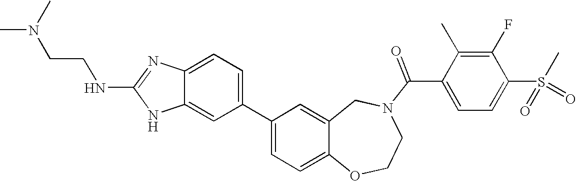 | N′-[6-(4-{[3-fluoro-2-methyl-4- (methylsulfonyl)phenyl]carbonyl}- 2,3,4,5-tetrahydro-1,4- benzoxazepin-7-yl)-1H- benzimidazol-2-yl]-N,N- dimethylethane-1,2-diamine |
| 638 | 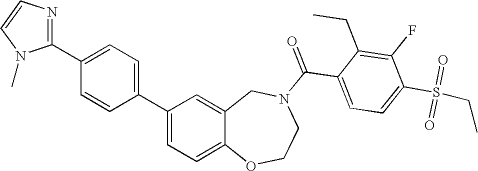 | 4-{[2-ethyl-4-(ethylsulfonyl)-3- fluorophenyl]carbonyl}-7-[4-(1- methyl-1H-imidazol-2- yl)phenyl]-2,3,4,5-tetrahydro- 1,4-benzoxazepine |
| 639 | 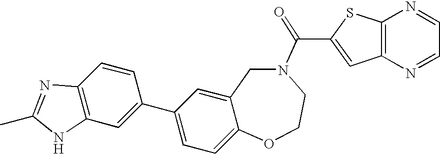 | 7-(2-methyl-1H-benzimidazol-6- yl)-4-(thieno[2,3-b]pyrazin-6- ylcarbonyl)-2,3,4,5-tetrahydro- 1,4-benzoxazepine |
| 640 | 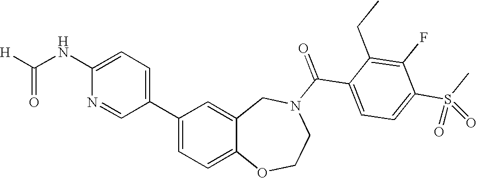 | N-[5-(4-{[2-ethyl-3-fluoro-4- (methylsulfonyl)phenyl]carbonyl}- 2,3,4,5-tetrahydro-1,4- benzoxazepin-7-yl)pyridin-2- yl]formamide |
| 641 | 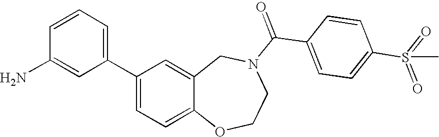 | 3-(4-{[4- (methylsulfonyl)phenyl]carbonyl}- 2,3,4,5-tetrahydro-1,4- benzoxazepin-7-yl)aniline |
| 642 | 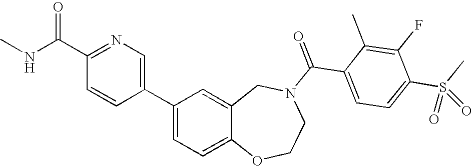 | 5-(4-{[3-fluoro-2-methyl-4- (methylsulfonyl)phenyl]carbonyl}- 2,3,4,5-tetrahydro-1,4- benzoxazepin-7-yl)-N- methylpyridine-2-carboxamide |
| 643 | 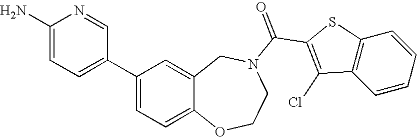 | 5-{4-[(3-chloro-1-benzothien-2- yl)carbonyl]-2,3,4,5-tetrahydro- 1,4-benzoxazepin-7-yl}pyridin- 2-amine |
| 644 | 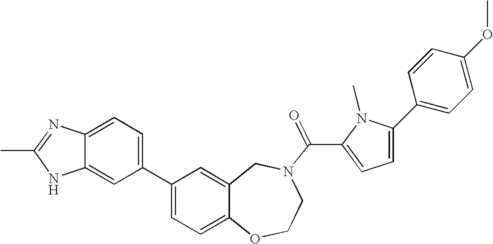 | 7-(2-methyl-1H-benzimidazol-6- yl)-4-({l-methyl-5-[4- (methyloxy)phenyl]-1H-pyrrol- 2-yl}carbonyl)-2,3,4,5- tetrahydro-1,4-benzoxazepine |
| 645 | 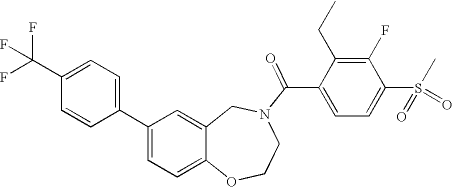 | 4-{[2-ethyl-3-fluoro-4- (methylsulfony])phenyl]carbonyl}- 7-[4-(trifluoromethyl)phenyl]- 2,3,4,5-tetrahydro-1,4- benzoxazepine |
| 646 | 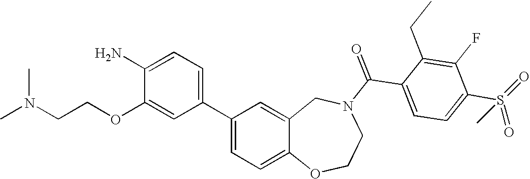 | 2-{[2- (dimethylamino)ethyl]oxy}-4- (4-{[2-ethyl-3-fluoro-4- (methylsulfonyl)phenyl]carbonyl}- 2,3,4,5-tetrahydro-1,4- benzoxazepin-7-yl)aniline |
| 647 | 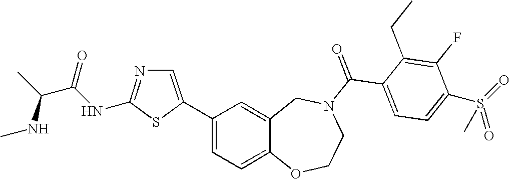 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]-N²-methyl-L-alaninamide |
| 648 | 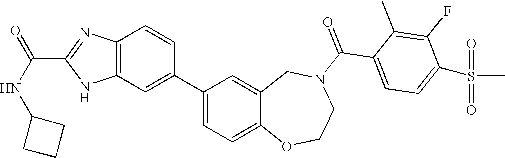 | N-cyclobutyl-6-(4-{[3-fluoro-2- methyl-4- (methylsulfony])phenyl]carbonyl}- 2,3,4,5-tetrahydro-1,4- benzoxazepin-7-yl)-1H- benzimidazole-2-carboxamide |
| 649 | 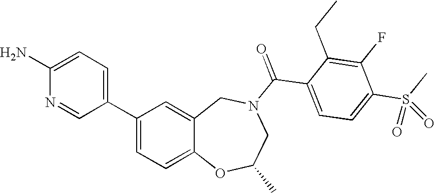 | 5-[(2S)-4-{[2-ethyl-3-fluoro-4- (methylsulfonyl)phenyl]carbonyl}- 2-methyl-2,3,4,5-tetrahydro- 1,4-benzoxazepin-7-yl]pyridin- 2-amine |
| 650 | 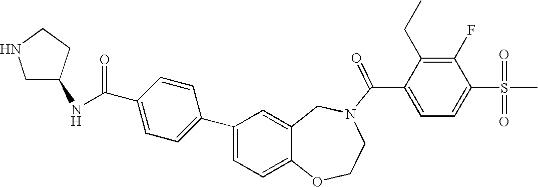 | 4-(4-{[2-ethyl-3-fluoro-4- (methylsulfonyl)phenyl]carbonyl}- 2,3,4,5-tetrahydro-1,4- benzoxazepin-7-yl)-N-[(3R)- pyrrolidin-3-yl]benzamide |
| 651 | 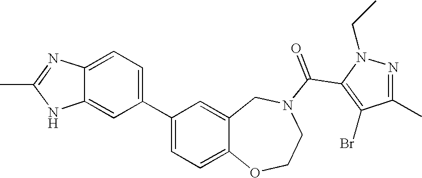 | 4-[(4-bromo-1-ethyl-3-methyl- 1H-pyrazol-5-yl)carbonyl]-7-(2- methyl-1H-benzimidazol-6-yl)- 2,3,4,5-tetrahydro-1,4- benzoxazepine |
| 652 | 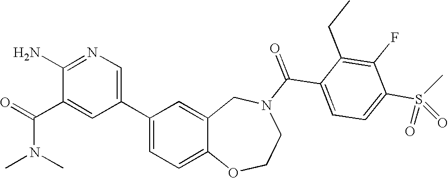 | 2-amino-5-(4-{[2-ethyl-3-fluoro-4- (methylsulfonyl)phenyl]carbonyl}- 2,3,4,5-tetrahydro-1,4- benzoxazepin-7-yl)-N,N- dimethylpyridine-3-carboxamide |
| 653 | 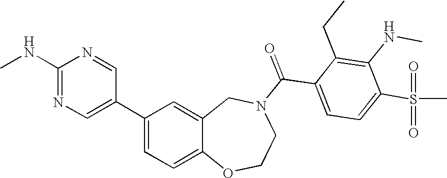 | 5-(4-{[2-ethyl-3-(methylamino)-4- (methylsulfonyl)phenyl]carbonyl}- 2,3,4,5-tetrahydro-1,4- benzoxazepin-7-yl)-N- methylpyrimidin-2-amine |
| 654 | 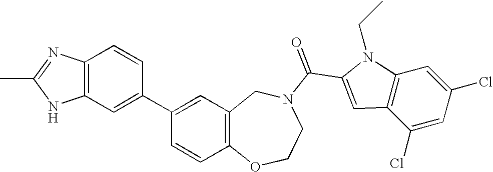 | 4-[(4,6-dichloro-1-ethyl-1H- indol-2-yl)carbonyl]-7-(2- methyl-1H-benzimidazol-6-yl)- 2,3,4,5-tetrahydro-1,4- benzoxazepine |
| 655 | 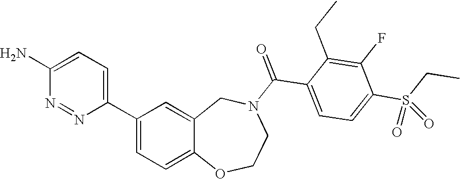 | 6-(4-{[2-ethyl-4-(ethylsulfonyl)- 3-fluorophenyl]carbonyl}- 2,3,4,5-tetrahydro-1,4- benzoxazepin-7-yl)pyridazin-3- amine |
| 656 | 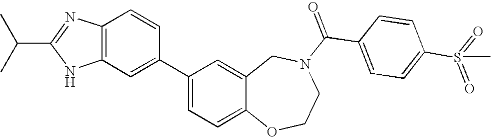 | 7-[2-(1-methylethyl)-1H- benzimidazol-6-yl]-4-{[4- (methylsulfonyl)phenyl]carbonyl}- 2,3,4,5-tetrahydro-1,4- benzoxazepine |
| 657 | 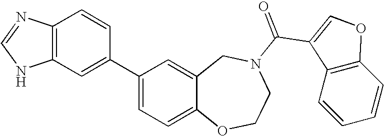 | 7-(1H-benzimidazol-6-yl)-4-(1- benzofuran-3-ylcarbonyl)- 2,3,4,5-tetrahydro-1,4- benzoxazepine |
| 658 | 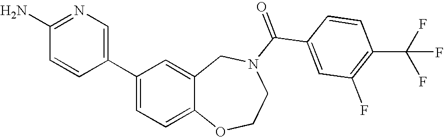 | 5-(4-{[3-fluoro-4- (trifluoromethyl)phenyl]carbonyl}- 2,3,4,5-tetrahydro-1,4- benzoxazepin-7-yl)pyridin-2- amine |
| 659 | 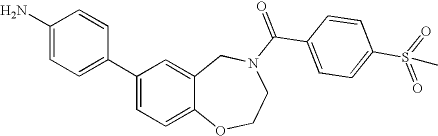 | 4-(4-{[4- (methylsulfonyl)phenyl]carbonyl}- 2,3,4,5-tetrahydro-l,4- benzoxazepin-7-yl)aniline |
| 660 | 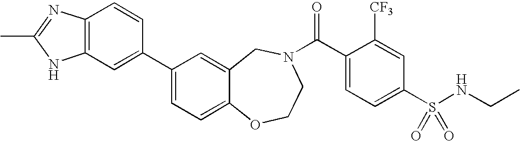 | N-ethyl-4-{[7-(2-methyl-1H- benzimidazol-6-yl)-2,3-dihydro- 1,4-benzoxazepin-4(5H)- yl]carbonyl}-3-(trifluoromethyl) benzenesulfonamide |
| 661 | 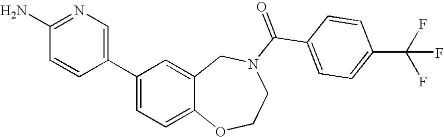 | 5-(4-{[4- (trifluoromethyl)phenyl]carbonyl}- 2,3,4,5-tetrahydro-1,4- benzoxazepin-7-yl)pyridin-2- amine |
| 662 | 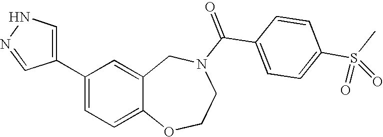 | 4-{[4- (methylsulfonyl)phenyl]carbonyl}- 7-(1H-pyrazol-4-yl)-2,3,4,5- tetrahydro-1,4-benzoxazepine |
| 663 | 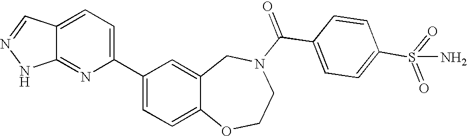 | 4-{[7-(1H-pyrazolo[3,4- b]pyridin-6-yl)-2,3-dihydro-1,4- benzoxazepin-4(5H)- yl]carbonyl}benzenesulfonamide |
| 664 | 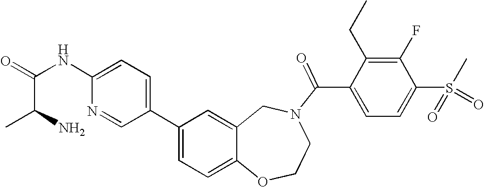 | N-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- L-alaninamide |
| 665 | 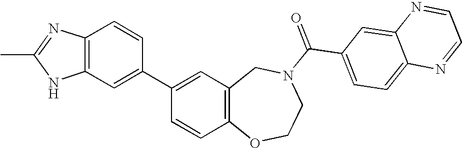 | 7-(2-methyl-1H-benzimidazol-6- yl)-4-(quinoxalin-6-ylcarbonyl)- 2,3,4,5-tetrahydro-1,4- benzoxazepine |
| 666 | 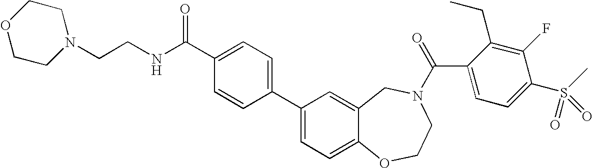 | 4-(4-{[2-ethyl-3-fluoro-4- (methylsulfonyl)phenyl]carbonyl}- 2,3,4,5-tetrahydro-1,4- benzoxazepin-7-yl)-N-(2- morpholin-4-ylethyl)benzamide |
| 667 | 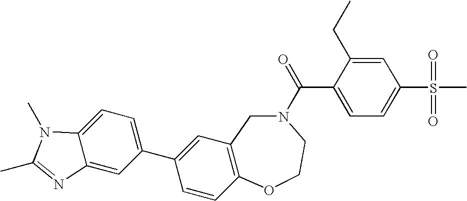 | 7-(1,2-dimethyl-1H- benzimidazol-5-yl)-4-{[2-ethyl-4- (methylsulfonyl)phenyl]carbonyl}- 2,3,4,5-tetrahydro-1,4- benzoxazepine |
| 668 | 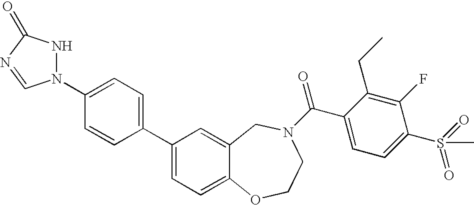 | l-[4-(4-{[2-ethyl-3-fluoro-4- (methylsulfonyl)phenyl]carbonyl}- 2,3,4,5-tetrahydro-1,4- benzoxazepin-7-yl)phenyl]-1,2- dihydro-3H-l,2,4-triazol-3-one |
| 669 | 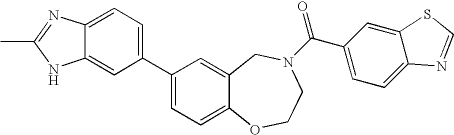 | 4-(1,3-benzothiazol-6- ylcarbonyl)-7-(2-methyl-1H- benzimidazol-6-yl)-2,3,4,5- tetrahydro-1,4-benzoxazepine |
| 670 | 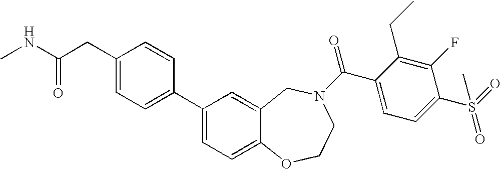 | 2-[4-(4-{[2-ethyl-3-fluoro-4- (methylsulfonyl)phenyl]carbonyl}- 2,3,4,5-tctrahydro-1,4- benzoxazepin-7-yl)phenyl]-N- methylacetamide |
| 671 | 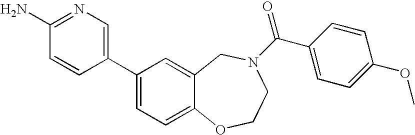 | 5-(4-{[4- (methyloxy)phenyl]carbonyl}- 2,3,4,5-tetrahydro-1,4- benzoxazepin-7-yl)pyridin-2- amine |
| 672 | 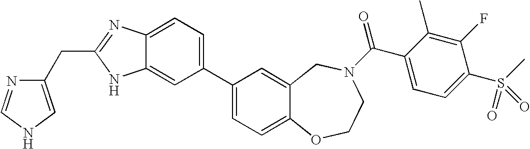 | 4-{[3-fluoro-2-methyl-4- (methylsulfonyl)phenyl]carbonyl}- 7-[2-(1H-imidazol-4- ylmethyl)-1H-benzimidazol-6- yl]-2,3,4,5-tetrahydro-1,4- benzoxazepine |
| 673 | 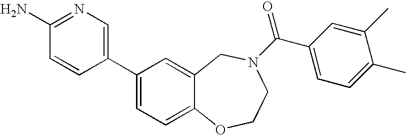 | 5-{4-[(3,4- dimethylphenyl)carbonyl]- 2,3,4,5-tetrahydro-1,4- benzoxazepin-7-yl}pyridin-2- amine |
| 674 | 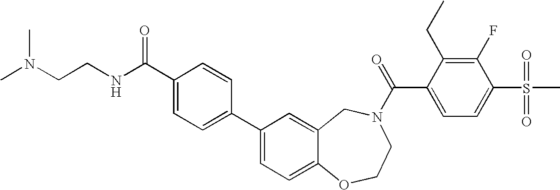 | N-[2-(dimethylamino)ethyl]-4- (4-{[2-ethyl-3-fluoro-4- (methylsulfonyl)phenyl]carbonyl}- 2,3,4,5-tetrahydro-1,4- benzoxazepin-7-yl)benzamide |
| 675 | 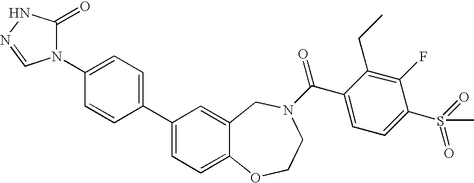 | 4-[4-(4-{[2-ethyl-3-fluoro-4- (methylsulfonyl)phenyl]carbonyl}- 2,3,4,5-tetrahydro-1,4- benzoxazepin-7-yl)phenyl]-2,4- dihydro-3H-1,2,4-triazol-3-one |
| 676 | 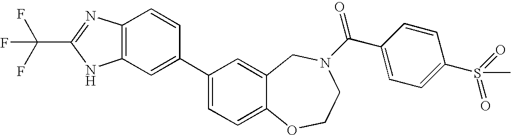 | 4-{[4- (methylsulfonyl)phenyl]carbonyl}- 7-[2-(trifluoromethyl)-1H- benzimidazol-6-yl]-2,3,4,5- tetrahydro-1,4-benzoxazepine |
| 677 | 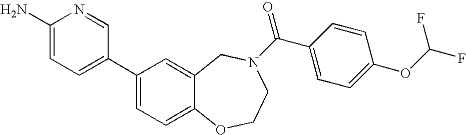 | 5-[4-({4- [(difluoromethyl)oxy]phenyl} carbonyl)-2,3,4,5-tetrahydro-1,4- benzoxazepin-7-yl]pyridin-2- amine |
| 678 | 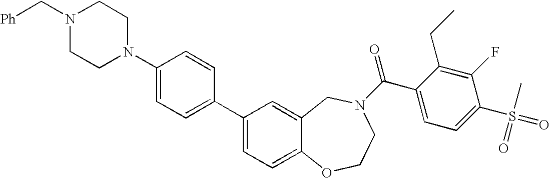 | 4-{[2-ethyl-3-fluoro-4- (methylsulfonyl)phenyl]carbonyl}- 7-{4-[4- (phenylmethyl)piperazin-1- yl]phenyl}-2,3,4,5-tetrahydro- 1,4-benzoxazepine |
| 679 | 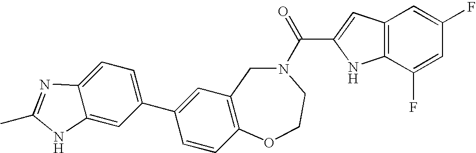 | 4-[(5,7-difluoro-1H-indol-2- yl)carbonyl]-7-(2-methyl-1H- benzimidazol-6-yl)-2,3,4,5- tetrahydro-1,4-benzoxazepine |
| 680 | 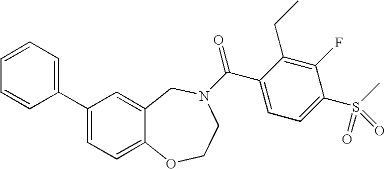 | 4-{[2-ethyl-3-fluoro-4- (methylsulfonyl)phenyl]carbonyl}- 7-phenyl-2,3,4,5-tetrahydro- 1,4-benzoxazepine |
| 681 | 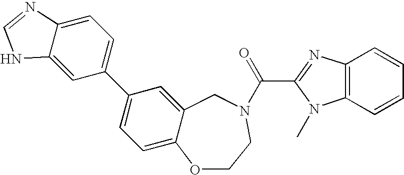 | 7-(1H-benzimidazol-6-yl)-4-[(1- methyl-1H-benzimidazol-2- yl)carbonyl]-2,3,4,5-tetrahydro- 1,4-benzoxazepine |
| 682 | 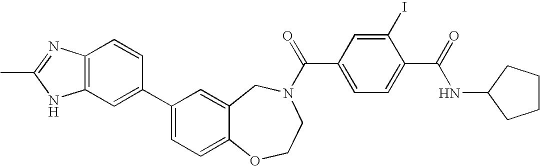 | N-cyclopentyl-2-iodo-4-{[7-(2- methyl-1H-benzimidazol-6-yl)- 2,3-dihydro-1,4-benzoxazepin- 4(5H)-yl]carbonyl}benzamide |
| 683 | 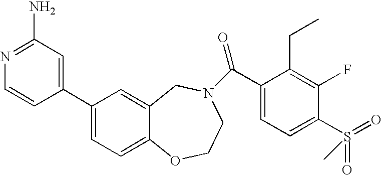 | 4-(4-{[2-ethyl-3-fluoro-4- (methylsulfonyl)phenyl]carbonyl}- 2,3,4,5-tetrahydro-1,4- benzoxazepin-7-yl)pyridin-2- amine |
| 684 | 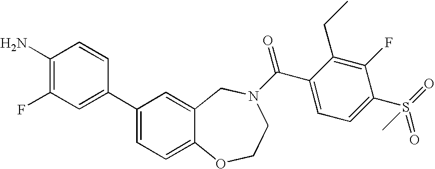 | 4-(4-{[2-ethyl-3-fluoro-4- (methylsulfonyl)phenyl]carbonyl}- 2,3,4,5-tetrahydro-1,4- benzoxazepin-7-yl)-2- fluoroaniline |
| 685 | 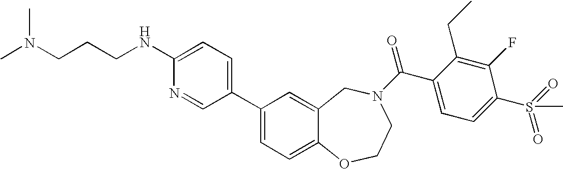 | N′-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- N,N-dimethylpropane-1,3- diamine |
| 686 | 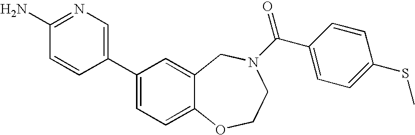 | 5-(4-{[4- (methylthio)phenyl]carbonyl}- 2,3,4,5-tetrahydro-1,4- benzoxazepin-7-yl)pyridin-2- amine |
| 687 | 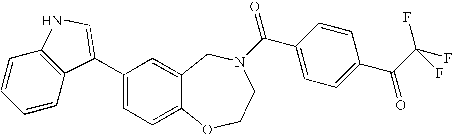 | 2,2,2-trifluoro-1-(4-{[7-(1H- indol-3-yl)-2,3-dihydro-1,4- benzoxazepin-4(5H)- yl]carbonyl}phenyl)ethanone |
| 688 | 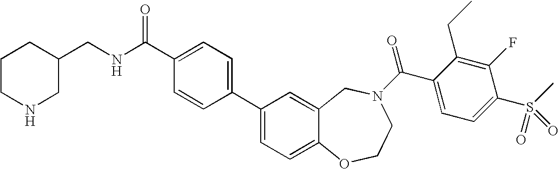 | 4-(4-{[2-ethyl-3-fluoro-4- (methylsulfonyl)phenyl]carbonyl}- 2,3,4,5-tetrahydro-1,4- benzoxazepin-7-yl)-N- (piperidin-3- ylmethyl)benzamide |
| 689 | 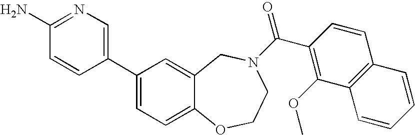 | 5-(4-{[1-(methyloxy)naphthalen- 2-yl]carbonyl}-2,3,4,5- tetrahydro-1,4-benzoxazepin-7- yl)pyridin-2-amine |
| 690 | 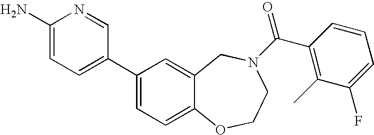 | 5-{4-[(3-fluoro-2- methylphenyl)carbonyl]-2,3,4,5- tetrahydro-1,4-benzoxazepin-7- yl}pyridin-2-amine |
| 691 | 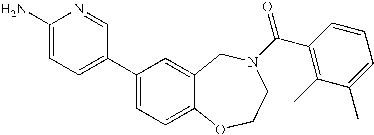 | 5-{4-[(2,3- dimethylphenyl)carbonyl]- 2,3,4,5-tetrahydro-1,4- benzoxazepin-7-yl}pyridine- amine |
| 692 | 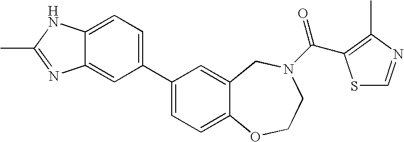 | 7-(2-methyl-1H-benzimidazol-5- yl)-4-[(4-methyl-1,3-thiazol-5- yl)carbonyl]-2,3,4,5-tetrahydro- 1,4-benzoxazepine |
| 693 | 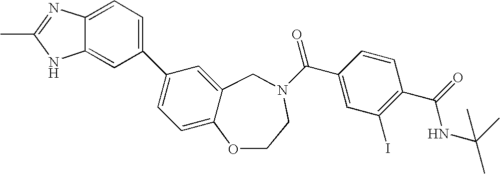 | N-(1,1-dimethylethyl)-2-iodo-4- {[7-(2-methyl-1H-benzimidazol- 6-yl)-2,3-dihydro-1,4- benzoxazepin-4(5H)- yl]carbonyl}benzamide |
| 694 | 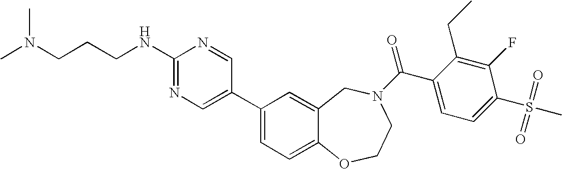 | N′-[5-(4-{[2-ethyl-3-fluoro-4- (methylsulfonyl)phenyl]carbonyl}- 2,3,4,5-tetrahydro-1,4- benzoxazepin-7-yl)pyrimidin-2- yl]-N,N-dimethylpropane-1,3- diamine |
| 695 | 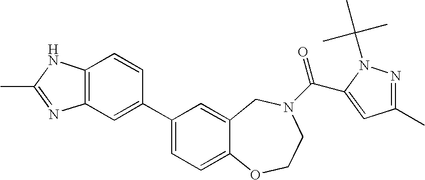 | 4-{[1-(1,1-dimethylethyl)-3- methyl-1H-pyrazol-5- yl]carbonyl}-7-(2-methyl-1H- benzimidazol-5-yl)-2,3,4,5- tetrahydro-1,4-benzoxazepine |
| 696 | 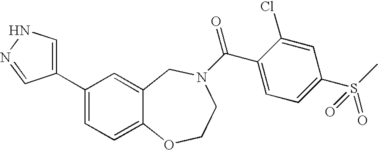 | 4-{[2-chloro-4- (methylsulfonyl)phenyl]carbonyl}- 7-(1H-pyrazol-4-yl)-2,3,4,5- tetrahydro-1,4-benzoxazepine |
| 697 | 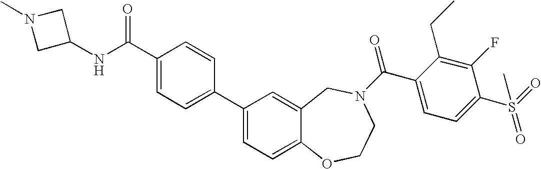 | 4-(4-{[2-ethyl-3-fluoro-4- (methylsulfonyl)phenyl]carbonyl}- 2,3,4,5-tetrahydro-1,4- benzoxazepin-7-yl)-N-(1- methylazetidin-3-yl)benzamide |
| 698 | 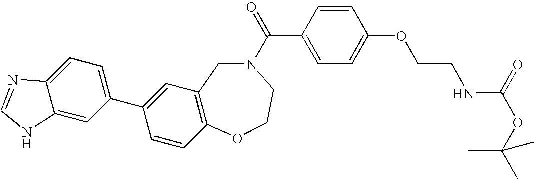 | 1,1-dimethylethyl{2-[(4-{[7- (1H-benzimidazol-6-yl)-2,3- dihydro-1,4-benzoxazepin- 4(5H)- yl]carbonyl}phenyl)oxy]ethyl} carbamate |
| 699 | 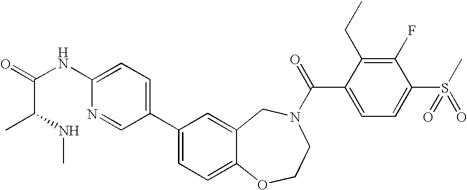 | N-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- N²-methyl-D-alaninamide |
| 700 | 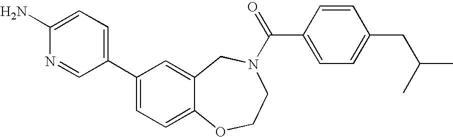 | 5-(4-{[4-(2- methylpropyl)phenyl]carbonyl}- 2,3,4,5-tetrahydro-1,4- benzoxazepin-7-yl)pyridin-2- amine |
| 701 | 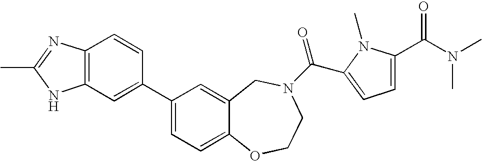 | N,N,1-trimethyl-5-{[7-(2- methyl-1H-benzimidazol-6-yl)- 2,3-dihydro-1,4-benzoxazepin- 4(5H)-yl]carbonyl}-1H-pyrrole- 2-carboxamide |
| 702 | 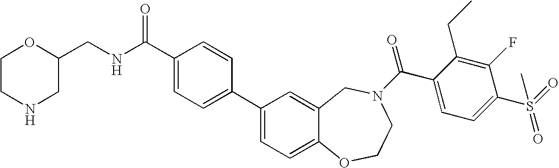 | 4-(4-{[2-ethyl-3-fluoro-4- (methylsulfonyl)phenyl]carbonyl}- 2,3,4,5-tetrahydro-1,4- benzoxazepin-7-yl)-N- (morpholin-2- ylmethyl)benzamide |
| 703 | 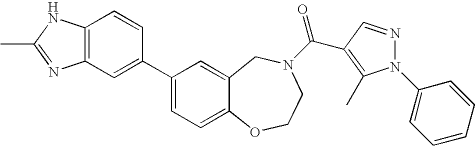 | 7-(2-methyl-1H-benzimidazol-5- yl)-4-[(5-methyl-1-phenyl-1H- pyrazol-4-yl)carbonyl]-2,3,4,5- tetrahydro-1,4-benzoxazepine |
| 704 | 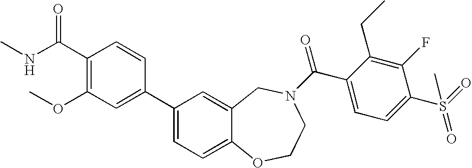 | 4-(4-{[2-ethyl-3-fluoro-4- (methylsulfonyl)phenyl]carbonyl}- 2,3,4,5-tetrahydro-1,4- benzoxazepin-7-yl)-N-methyl-2- (methyloxy)benzamide |
| 705 | 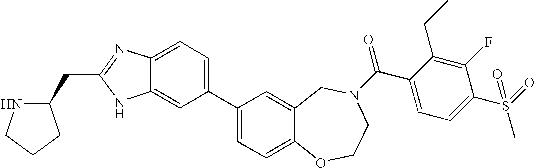 | 4-{[2-ethyl-3-fluoro-4- (methylsulfonyl)phenyl]carbonyl}- 7-{2-[(2R)-pyrrolidin-2- ylmethyl]-1H-benzimidazol-6- yl}-2,3,4,5-tetrahydro- 1,4-benzoxazepine |
| 706 | 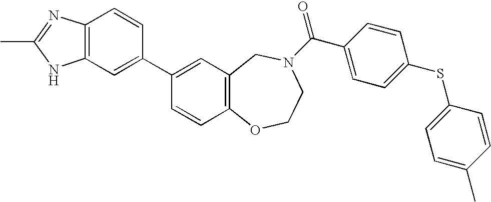 | 7-(2-methyl-1H-benzimidazol-6- yl)-4-({4-[(4- methylphenyl)thio]phenyl} carbonyl)-2,3,4,5-tetrahydro-1,4- benzoxazepine |
| 707 | 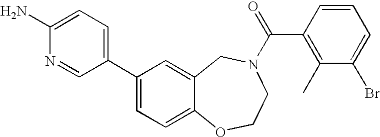 | 5-{4-[(3-bromo-2- methylphenyl)carbonyl]-2,3,4,5- tetrahydro-1,4-benzoxazepin-7- yl}pyridin-2-amine |
| 708 | 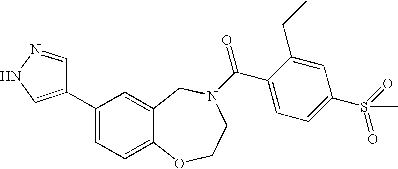 | 4-{[2-ethyl-4- (methylsulfonyl)phenyl]carbonyl}- 7-(1H-pyrazol-4-yl)-2,3,4,5- tetrahydro-1,4-benzoxazepine |
| 709 | 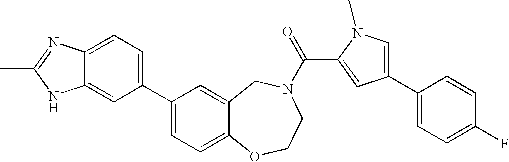 | 4-{[4-(4-fluorophenyl)-1- methyl-1H-pyrrol-2- yl]carbonyl}-7-(2-methyl-1H- benzimidazol-6-yl)-2,3,4,5- tetrahydro-1,4-benzoxazepine |
| 710 | 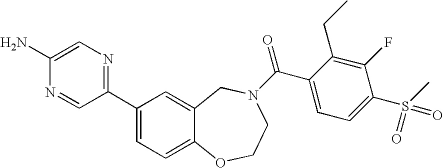 | 5-(4-{[2-ethyl-3-fluoro-4- (methylsulfonyl)phenyl]carbonyl}- 2,3,4,5-tetrahydro-1,4- benzoxazepin-7-yl)pyrazin-2- amine |
| 711 | 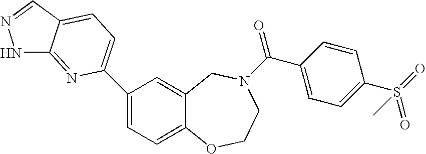 | 4-{[4- (methylsulfonyl)phenyl]carbonyl}- 7-(1H-pyrazolo[3,4-b]pyridin- 6-yl)-2,3,4,5-tetrahydro-1,4- bcnzoxazepine |
| 712 | 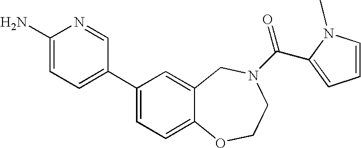 | 5-{4-[(1-methyl-1H-pyrrol-2- yl)carbonyl]-2,3,4,5-tetrahydro- 1,4-benzoxazepin-7-yl}pyridin- 2-amine |
| 713 | 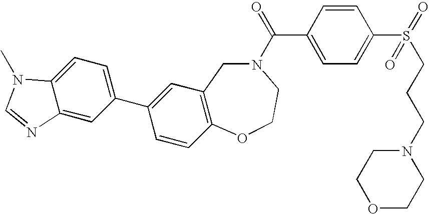 | 7-(1-methyl-1H-benzimidazol-5- yl)-4-({4-[(3-morpholin-4- ylpropyl)sulfonyl]phenyl} carbonyl)-2,3,4,5-tetrahydro-1,4- benzoxazepine |
| 714 | 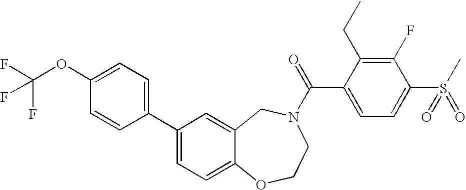 | 4-{[2-ethyl-3-fluoro-4- (methylsulfonyl)phenyl]carbonyl}- 7-{4- [(trifluoromethyl)oxy]phenyl}- 2,3,4,5-tetrahydro-1,4- benzoxazepine |
| 715 | 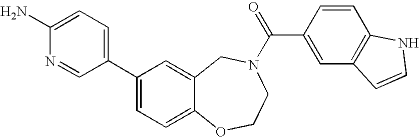 | 5-[4-(1H-indol-5-ylcarbonyl)- 2,3,4,5-tetrahydro-1,4- benzoxazepin-7-yl]pyridin-2- amine |
| 716 | 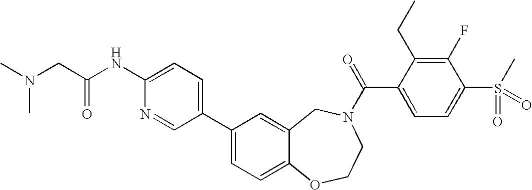 | N-[5-(4-{[2-ethyl-3-fluoro-4- (mcthylsulfonyl)phenyl]carbonyl}- 2,3,4,5-tetrahydro-1,4- benzoxazepin-7-yl)pyridin-2-yl]- N²,N²-dimethylglycinamide |
| 717 | 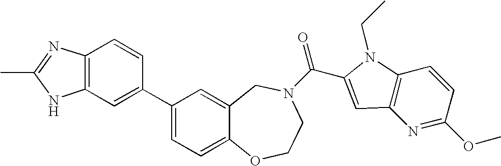 | 4-{[1-ethyl-5-(methyloxy)-1H- pyrrolo[3,2-b]pyridin-2- yl]carbonyl}-7-(2-methyl-1H- benzimidazol-6-yl)-2,3,4,5- tetrahydro-1,4-benzoxazepine |
| 718 | 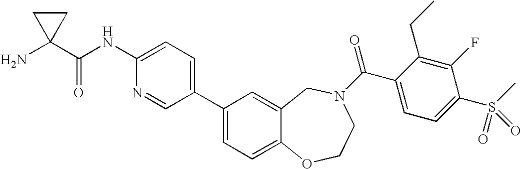 | 1-amino-N-[5-(4-{[2-ethyl-3- fluoro-4- (methylsulfonyl)phenyl]carbonyl}- 2,3,4,5-tetrahydro-1,4- benzoxazepin-7-yl)pyridin-2- yl]cyclopropancarboxamide |
| 719 | 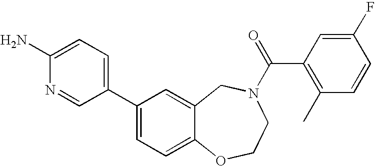 | 5-{4-[(5-fluoro-2- methylphenyl)carbonyl]-2,3,4,5- tetrahydro-1,4-benzoxazepin-7- yl}pyridin-2-amine |
| 720 | 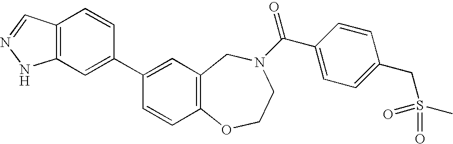 | 7-(1H-indazol-6-yl)-4-({4- [(methylsulfonyl)methyl]phenyl} carbonyl)-2,3,4,5-tetrahydro- 1,4-benzoxazepine |
| 721 | 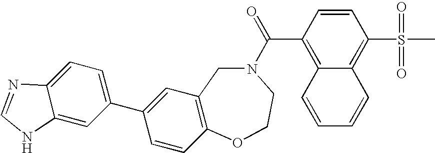 | 7-(1H-benzimidazol-6-yl)-4-{[4- (methylsulfonyl)naphthalen-1- yl]carbonyl}-2,3,4,5-tetrahydro- 1,4-benzoxazepine |
| 722 | 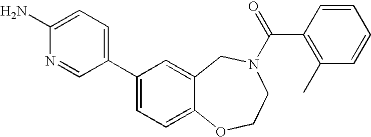 | 5-{4-[(2- methylphenyl)carbonyl]-2,3,4,5- tetrahydro-1,4-benzoxazepin-7- yl}pyridin-2-amine |
| 723 | 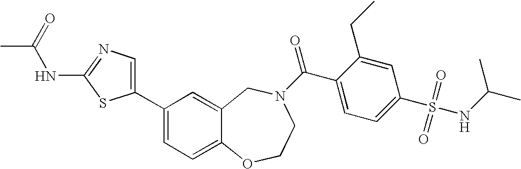 | N-(5-{4-[(2-ethyl-4-{[(1- methylethyl)amino]sulfonyl} phenyl)carbonyl]-2,3,4,5-tetrahydro- 1,4-benzoxazepin-7-yl}-1,3- thiazol-2-yl)acetamide |
| 724 | 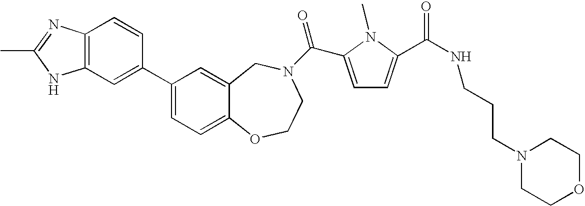 | 1-methyl-5-{[7-(2-methyl-1H- benzimidazol-6-yl)-2,3-dihydro- 1,4-benzoxazepin-4(5H)- yl]carbonyl}-N-(3-morpholin-4- ylpropyl)-1H-pyrrole-2- carboxamide |
| 725 | 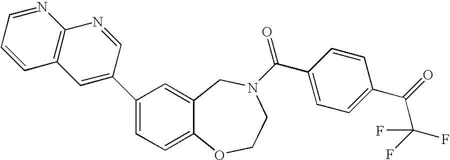 | 2,2,2-trifluoro-1-(4-{[7-(1,8- naphthyridin-3-yl)-2,3-dihydro- 1,4-benzoxazepin-4(5H)- yl]carbonyl}phenyl)ethanone |
| 726 | 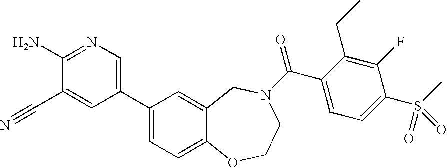 | 2-amino-5-(4-{[2-ethyl-3-fluoro-4- (methylsulfonyl)phenyl]carbonyl}- 2,3,4,5-tetrahydro-1,4- benzoxazepin-7-yl)pyridine-3- carbonitrile |
| 727 | 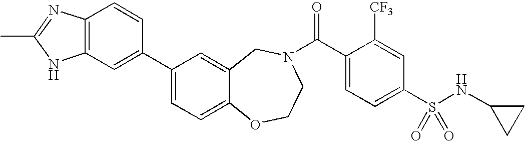 | N-cyclopropyl-4-{[7-(2-methyl- 1H-benzimidazol-6-yl)-2,3- dihydro-1,4-benzoxazepin- 4(5H)-yl]carbonyl}-3- (trifluoromethyl) benzenesulfonamide |
| 728 | 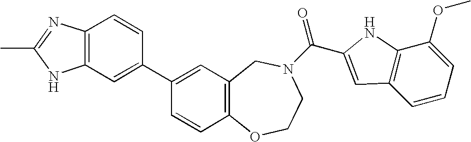 | 7-(2-methyl-1H-benzimidazol-6- yl)-4-{[7-(methyloxy)-1H-indol- 2-yl]carbonyl}-2,3,4,5- tetrahydro-1,4-benzoxazepine |
| 729 | 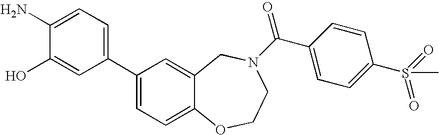 | 2-amino-5-(4-{[4- (methylsulfonyl)phenyl]carbonyl}- 2,3,4,5-tetrahydro-1,4- benzoxazepin-7-yl)phenol |
| 730 | 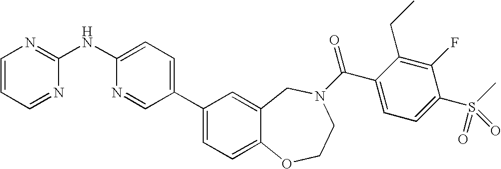 | N-[5-(4-{[2-ethyl-3-fluoro-4- (methylsulfonyl)phenyl]carbonyl}- 2,3,4,5-tetrahydro-1,4- benzoxazepin-7-yl)pyridin-2- yl]pyrimidin-2-amine |
| 731 | 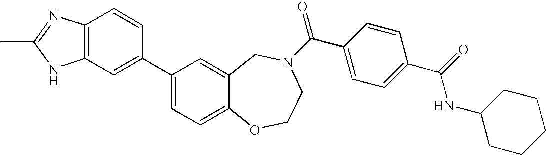 | N-cyclohexyl-4-{[7-(2-methyl- 1H-benzimidazol-6-yl)-2,3- dihydro-1,4-benzoxazepin- 4(5H)-yl]carbonyl}benzamide |
| 732 | 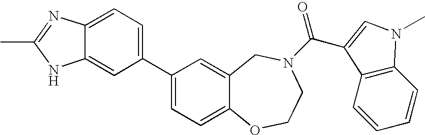 | 7-(2-methyl-1H-benzimidazol-6- yl)-4-[(1-methyl-1H-indol-3- yl)carbonyl]-2,3,4,5-tetrahydro- 1,4-benzoxazepine |
| 733 | 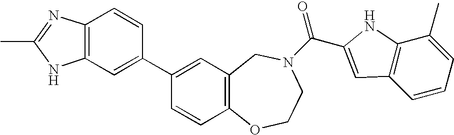 | 7-(2-methyl-1H-benzimidazol-6- yl)-4-[(7-methyl-1H-indol-2- yl)carbonyl]-2,3,4,5-tetrahydro- 1,4-benzoxazepine |
| 734 | 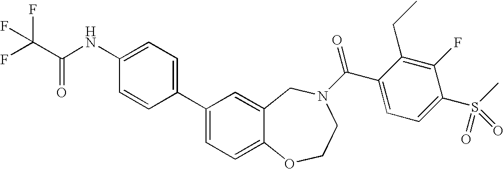 | N-[4-(4-{[2-ethyl-3-fluoro-4- (methylsulfonyl)phenyl]carbonyl}- 2,3,4,5-tetrahydro-1,4- benzoxazepin-7-yl)phenyl]- 2,2,2-trifluoroacetamide |
| 735 | 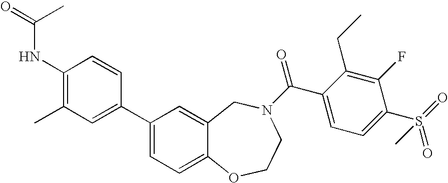 | N-[4-(4-{[2-ethyl-3-fluoro-4- (methylsulfonyl)phenyl]carbonyl}- 2,3,4,5-tetrahydro-1,4- benzoxazepin-7-yl)-2- methylphenyl]acetamide |
| 736 | 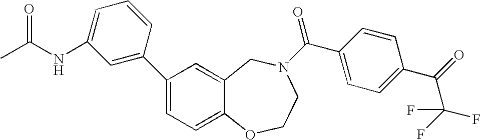 | N-[3-(4-{[4- (trifluoroacetyl)phenyl]carbonyl}- 2,3,4,5-tetrahydro-1,4- benzoxazepin-7- yl)phenyl]acetamide |
| 737 | 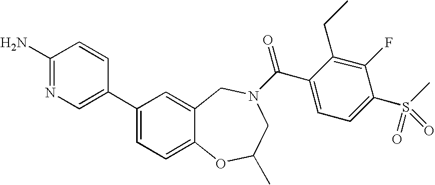 | 5-(4-{[2-ethyl-3-fluoro-4- (methylsulfonyl)phenyl]carbonyl}- 2-methyl-2,3,4,5-tetrahydro- 1,4-benzoxazepin-7-yl)pyridin- 2-amine |
| 738 | 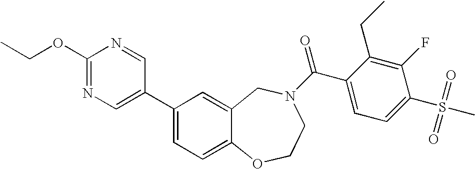 | 4-{[2-ethyl-3-fluoro-4- (methylsulfonyl)phenyl]carbonyl}- 7-[2-(ethyloxy)pyrimidin-5- yl]-2,3,4,5-tetrahydro-1,4- benzoxazepine |
| 739 | 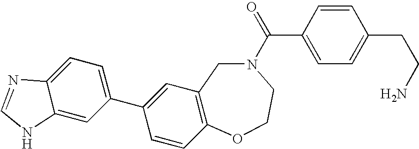 | 2-(4-{[7-(1H-benzimidazol-6- yl)-2,3-dihydro-1,4- benzoxazepin-4(5H)- yl]carbonyl}phenyl)ethanamine |
| 740 | 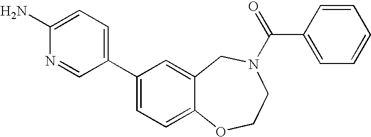 | 5-[4-(phenylcarbonyl)-2,3,4,5- tetrahydro-1,4-benzoxazepin-7- yl]pyridin-2-amine |
| 741 | 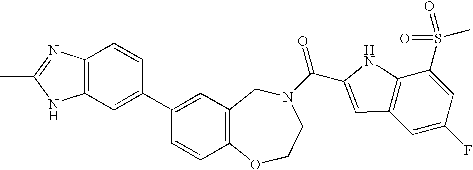 | 4-{[5-fluoro-7-(methylsulfonyl)- 1H-indol-2-yl]carbonyl}-7-(2- methyl-1H-benzimidazol-6-yl)- 2,3,4,5-tetrahydro-1,4- benzoxazepine |
| 742 | 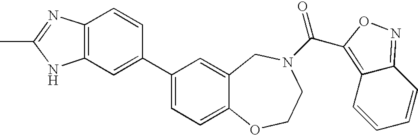 | 4-(2,1-benzisoxazol-3- ylcarbonyl)-7-(2-methyl-1H- benzimidazol-6-yl)-2,3,4,5- tetrahydro-1,4-benzoxazepine |
| 743 | 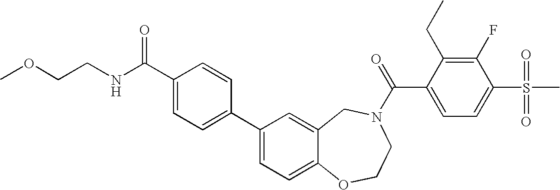 | 4-(4-{[2-ethyl-3-fluoro-4- (methylsulfonyl)phenyl]carbonyl}- 2,3,4,5-tetrahydro-1,4- benzoxazepin-7-yl)-N-[2- (methyloxy)ethyl]benzamide |
| 744 | 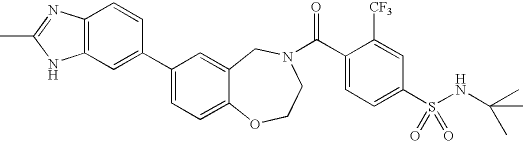 | N-(1,1-dimethylethyl)-4-{[7-(2-methyl-1H-benzimidazol-6-yl)- 2,3-dihydro-1,4-benzoxazepin-4(5H)-yl]carbonyl}-3-(trifluoromethyl) benzenesulfonamide |
| 745 | 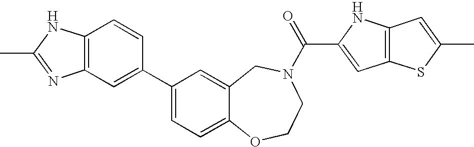 | 7-(2-methyl-1H-benzimidazol-5- yl)-4-[(2-methyl-4H-thieno[3,2- b]pyrrol-5-yl)carbonyl]-2,3,4,5- tetrahydro-1,4-benzoxazepine |
| 746 | 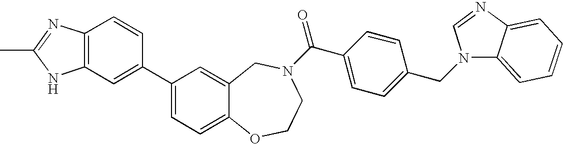 | 4-{[4-(1H-benzimidazol-1- ylmethyl)phenyl]carbonyl}-7-(2- methyl-1H-benzimidazol-6-yl)- 2,3,4,5-tetrahydro-1,4- benzoxazepine |
| 747 | 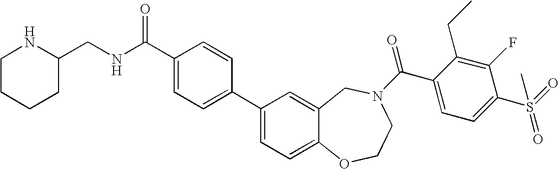 | 4-(4-{[2-ethyl-3-fluoro-4- (methylsulfonyl)phenyl] carbonyl}-2,3,4,5-tetrahydro- 1,4-benzoxazepin-7-yl)-N- (piperidin-2- ylmethyl)benzamide |
| 748 | 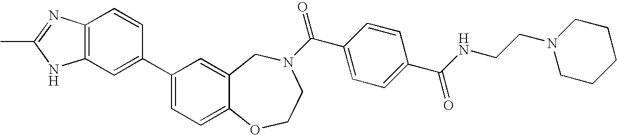 | 4-{[7-(2-methyl-1H- benzimidazol-6-yl)-2,3-dihydro- 1,4-benzoxazepin-4(5H)- yl]carbonyl}-N-(2-piperidin-1- ylethyl)benzamide |
| 749 | 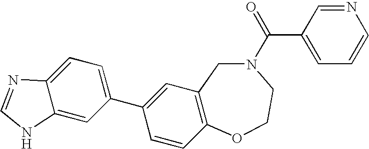 | 7-(1H-benzimidazol-6-yl)-4- (pyridin-3-ylcarbonyl)-2,3,4,5- tetrahydro-1,4-benzoxazepine |
| 750 | 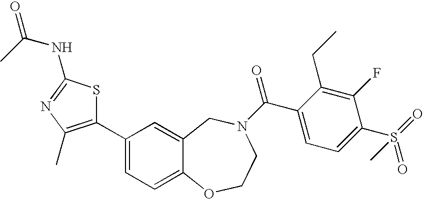 | N-[5-(4-{[2-ethyl-3-fluoro-4- (methylsulfonyl)phenyl]carbonyl}- 2,3,4,5-tetrahydro-1,4- benzoxazepin-7-yl)-4-methyl- 1,3-thiazol-2-yl]acetamide |
| 751 | 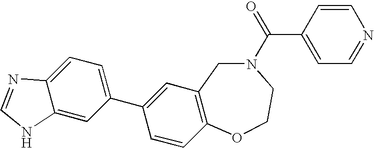 | 7-(1H-benzimidazol-6-yl)-4- (pyridin-4-ylcarbonyl)-2,3,4,5- tetrahydro-1,4-benzoxazepine |
| 752 | 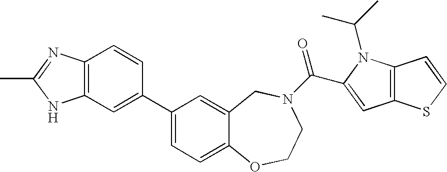 | 7-(2-methyl-1H-benzimidazol-6- yl)-4-{[4-(1-methylethyl)-4H- thieno[3,2-b]pyrrol-5- yl]carbonyl}-2,3,4,5-tetrahydro- 1,4-benzoxazepine |
| 753 | 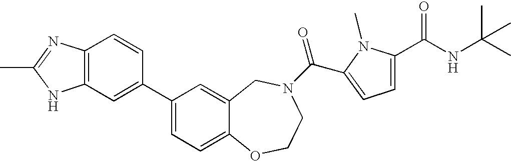 | N-(1,1-dimethylethyl)-1-methyl- 5-{[7-(2-methyl-1H- benzimidazol-6-yl)-2,3-dihydro- 1,4-benzoxazepin-4(5H)- yl]carbonyl}-1H-pyrrole-2- carboxamide |
| 754 | 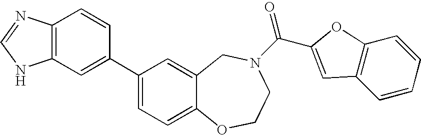 | 7-(1H-benzimidazol-6-yl)-4-(1- benzofuran-2-ylcarbonyl)- 2,3,4,5-tetrahydro-1,4- benzoxazepine |
| 755 | 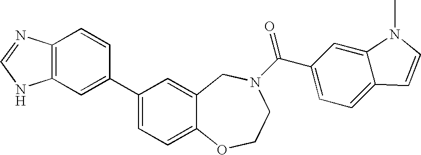 | 7-(1H-benzimidazol-6-yl)-4-[(1- methyl-1H-indol-6-yl)carbonyl]- 2,3,4,5-tetrahydro-1,4- benzoxazepine |
| 756 | 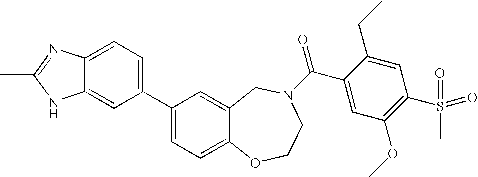 | 4-{[2-ethyl-5-(methyloxy)-4- (methylsulfonyl)phenyl]carbonyl}- 7-(2-methyl-1H-benzimidazol- 6-yl)-2,3,4,5-tetrahydro-1,4- benzoxazepine |
| 757 | 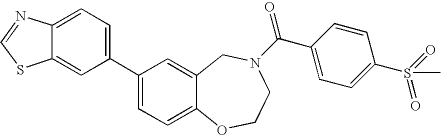 | 7-(1,3-benzothiazol-6-yl)-4-{[4- (methylsulfonyl)phenyl]carbonyl}- 2,3,4,5-tetrahydro-1,4- benzoxazepine |
| 758 | 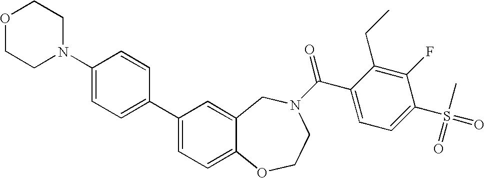 | 4-{[2-ethyl-3-fluoro-4- (methylsulfonyl)phenyl]carbonyl}- 7-(4-morpholin-4-ylphenyl)- 2,3,4,5-tetrahydro-1,4- benzoxazepine |
| 759 | 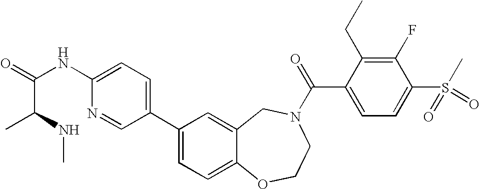 | N-[5-(4-{[2-ethyl-3-fluoro-4- (methylsulfonyl)phenyl]carbonyl}- 2,3,4,5-tetrahydro-1,4- benzoxazepin-7-yl)pyridin-2-yl]- N²-methyl-L-alaninamide |
| 760 | 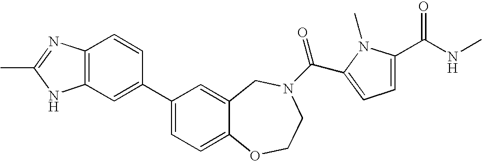 | N,1-dimethyl-5-{[7-(2-methyl- 1H-benzimidazol-6-yl)-2,3- dihydro-1,4-benzoxazepin- 4(5H)-yl]carbonyl}-1H-pyrrole- 2-carboxamide |
| 761 | 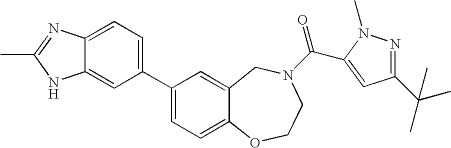 | 4-{[3-(1,1-dimethylethyl)-1- methyl-1H-pyrazol-5- yl]carbonyl}-7-(2-methyl-1H- benzimidazol-6-yl)-2,3,4,5- tetrahydro-1,4-benzoxazepine |
| 762 | 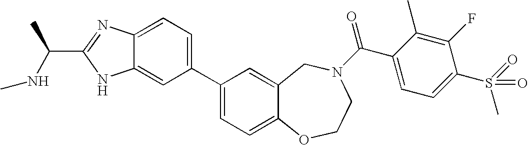 | (1S)-1-[6-(4-{[3-fluoro-2- methyl-4- (methylsulfonyl)phenyl]carbonyl}- 2,3,4,5-tetrahydro-1,4- benzoxazepin-7-yl)-1H- benzimidazol-2-yl]-N- methylethanamine |
| 763 | 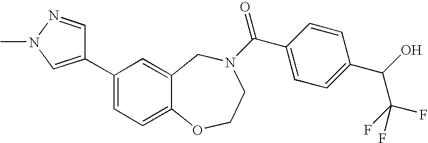 | 2,2,2-trifluoro-1-(4-{[7-(1- methyl-1H-pyrazol-4-yl)-2,3- dihydro-1,4-benzoxazepin- 4(5H)- yl]carbonyl}phenyl)ethanol |
| 764 | 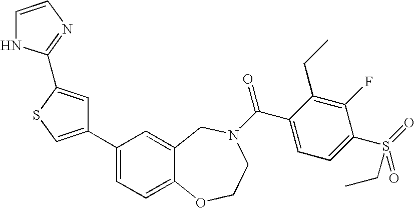 | 4-{[2-ethyl-4-(ethylsulfonyl)-3- fluorophenyl]carbonyl}-7-[5- (1H-imidazol-2-yl)-3-thienyl]- 2,3,4,5-tetrahydro-1,4- benzoxazepine |
| 765 | 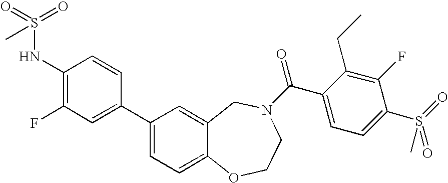 | N-[4-(4-{[2-ethyl-3-fluoro-4- (methylsulfonyl)phenyl]carbonyl}- 2,3,4,5-tetrahydro-1,4- benzoxazepin-7-yl)-2- fluorophenyl]methanesulfonamide |
| 766 | 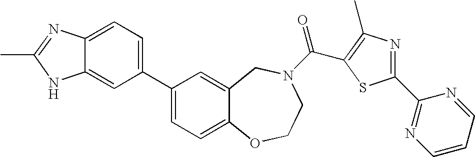 | 7-(2-methyl-1H-benzimidazol-6- yl)-4-[(4-methyl-2-pyrimidin-2- yl-1,3-thiazol-5-yl)carbonyl]- 2,3,4,5-tetrahydro-1,4- benzoxazepine |
| 767 | 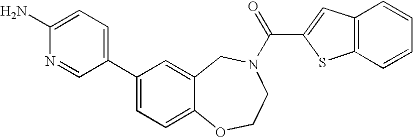 | 5-[4-(1-benzothien-2- ylcarbonyl)-2,3,4,5-tetrahydro- 1,4-benzoxazepin-7-yl]pyridin- 2-amine |
| 768 | 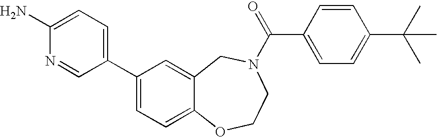 | 5-(4-{[4-(1,1- dimethylethyl)phenyl]carbonyl}- 2,3,4,5-tetrahydro-1,4- benzoxazepin-7-yl)pyridin-2- amine |
| 769 | 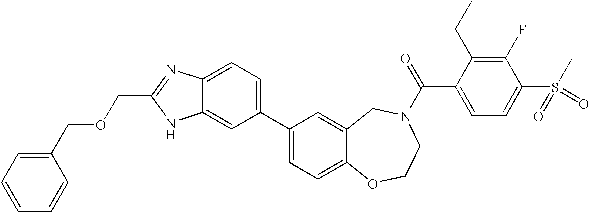 | 4-{[2-ethyl-3-fluoro-4- (methylsulfonyl)phenyl]carbonyl}- 7-(2- [(phenylmethyl)oxy]methyl}- 1H-benzimidazol-6-yl)-2,3,4,5- tetrahydro-1,4-benzoxazepine |
| 770 | 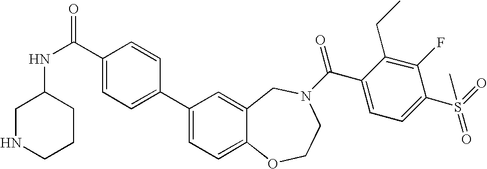 | 4-(4-{[2-ethyl-3-fluoro-4- (methylsulfonyl)phenyl]carbonyl}- 2,3,4,5-tetrahydro-1,4- benzoxazepin-7-yl)-N-piperidin- 3-ylbenzamide |
| 771 | 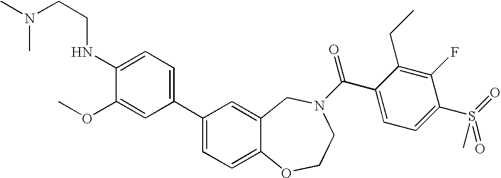 | N′-[4-(4-{[2-ethyl-3-fluoro-4- (methylsulfonyl)phenyl]carbonyl}- 2,3,4,5-tetrahydro-1,4- benzoxazepin-7-yl)-2- (methyloxy)phenyl]-N,N- dimethylethane-1,2-diamine |
| 772 | 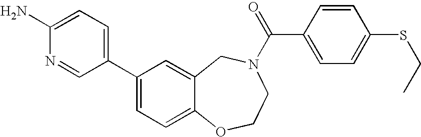 | 5-(4-{[4- (ethylthio)phenyl]carbonyl}- 2,3,4,5-tetrahydro-1,4- benzoxazepin-7-yl)pyridin-2- amine |
| 773 | 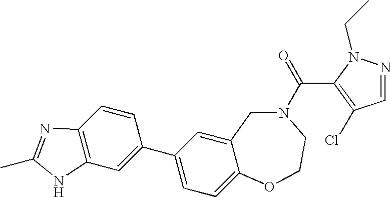 | 4-[(4-chloro-1-ethyl-1H-pyrazol- 5-yl)carbonyl]-7-(2-methyl-1H- benzimidazol-6-yl)-2,3,4,5- tetrahydro-1,4-benzoxazepine |
| 774 | 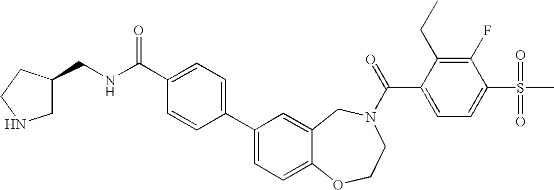 | 4-(4-{[2-ethyl-3-fluoro-4- (methylsulfonyl)phenyl]carbonyl}- 2,3,4,5-tetrahydro-1,4- benzoxazepin-7-yl)-N-[(3R)- pyrrolidin-3- ylmethyl]benzamide |
| 775 | 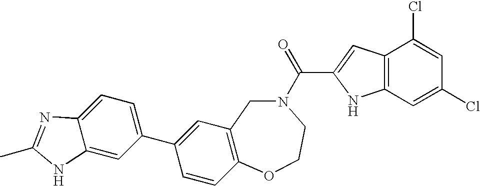 | 4-[(4,6-dichloro-1H-indol-2- yl)carbonyl]-7-(2-methyl-1H- benzimidazol-6-yl)-2,3,4,5- tetrahydro-1,4-benzoxazepine |
| 776 | 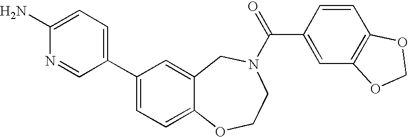 | 5-[4-(1,3-benzodioxol-5- ylcarbonyl)-2,3,4,5-tetrahydro- 1,4-benzoxazepin-7-yl]pyridin- 2-amine |
| 777 | 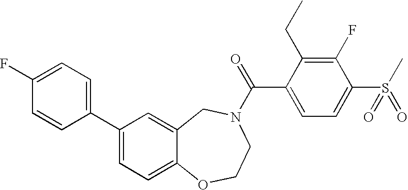 | 4-{[2-ethyl-3-fluoro-4- (methylsulfonyl)phenyl]carbonyl}- 7-(4-fluorophenyl)-2,3,4,5- tetrahydro-1,4-benzoxazepine |
| 778 | 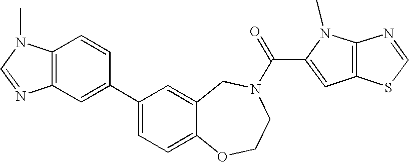 | 7-(1-methyl-lH-benzimidazol-5- yl)-4-[(4-methyl-4H-pyrrolo[2,3- d][1,3]thiazol-5-yl)carbonyl]- 2,3,4,5-tetrahydro-1,4- benzoxazepine |
| 779 | 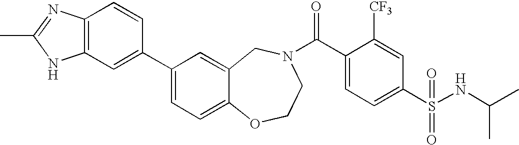 | 4-{[7-(2-methyl-1H- benzimidazol-6-yl)-2,3-dihydro- 1,4-benzoxazepin-4(5H)- yl]carbonyl}-N-(1-methylethyl)- 3-(trifluoromethyl) benzenesulfonamide |
| 780 | 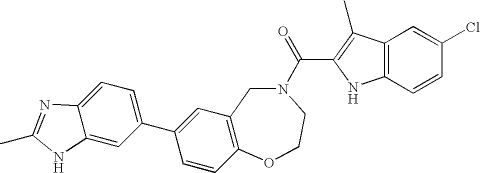 | 4-[(5-chloro-3-methyl-1H-indol- 2-yl)carbonyl]-7-(2-methyl-1H- benzimidazol-6-yl)-2,3,4,5- tetrahydro-1,4-benzoxazepine |
| 781 | 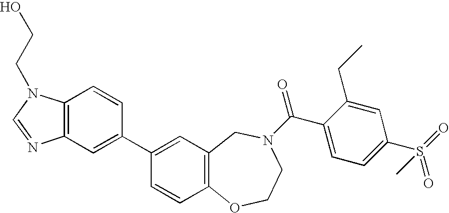 | 2-[5-(4-{[2-ethyl-4- (methylsulfonyl)phenyl]carbonyl}- 2,3,4,5-tetrahydro-1,4- benzoxazepin-7-yl)-1H- benzimidazol-1-yl]ethanol |
| 782 | 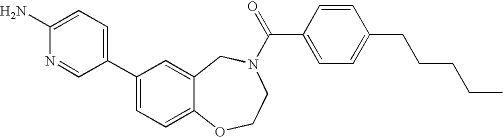 | 5-{4-[(4- pentylphenyl)carbonyl]-2,3,4,5- tetrahydro-1,4-benzoxazepin-7- yl}pyridin-2-amine |
| 783 | 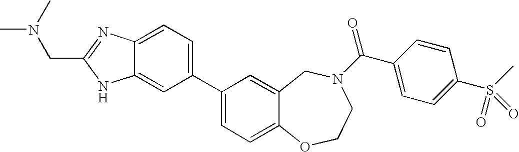 | N,N-dimethyl-1-[6-(4-{[4- (methylsulfonyl)phenyl]carbonyl}- 2,3,4,5-tetrahydro-1,4- benzoxazepin-7-yl)-1H- benzimidazol-2-yl]methanamine |
| 784 | 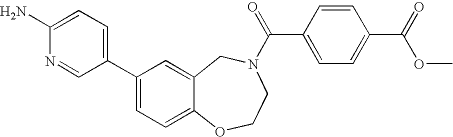 | methyl 4-{[7-(6-aminopyridin-3- yl)-2,3-dihydro-1,4- benzoxazepin-4(5H)- yl]carbonyl}benzoate |
| 785 | 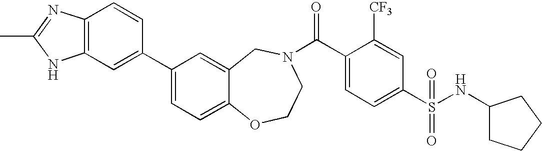 | N-cyclopentyl-4-{[7-(2-methyl- 1H-benzimidazol-6-yl)-2,3- dihydro-1,4-benzoxazepin- 4(5H)-yl]carbonyl}-3- (trifluoromethyl) benzenesulfonamide |
| 786 | 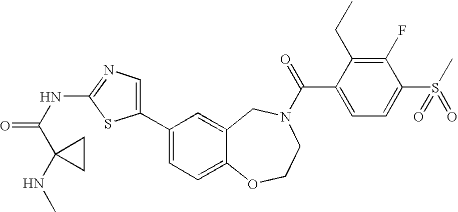 | N-[5-(4-{[2-ethyl-3-fluoro-4- (methylsulfonyl)phenyl]carbonyl}- 2,3,4,5-tetrahydro-1,4- benzoxazepin-7-yl)-1,3-thiazol- 2-yl]-1-(methylamino) cyclopropanecarboxamide |
| 787 | 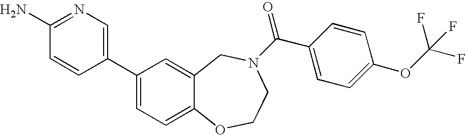 | 5-[4-({4- [(trifluoromethyl)oxy]phenyl} carbonyl)-2,3,4,5-tetrahydro-1,4- benzoxazepin-7-yl]pyridin-2- amine |
| 788 | 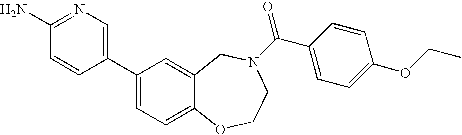 | 5-(4-{[4- (ethyloxy)phenyl]carbonyl}- 2,3,4,5-tetrahydro-1,4- benzoxazepin-7-yl)pyridin-2- amine |
| 789 | 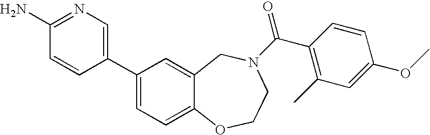 | 5-(4-{[2-methyl-4- (methyloxy)phenyl]carbonyl}- 2,3,4,5-tetrahydro-1,4- benzoxazepin-7-yl)pyridin-2- amine |
| 790 | 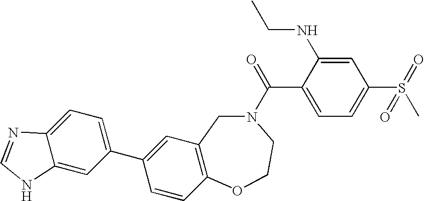 | 2-{[7-(1H-benzimidazol-6-yl)- 2,3-dihydro-1,4-benzoxazepin- 4(5H)-yl]carbonyl}-N-ethyl-5- (methylsulfonyl)aniline |
| 791 | 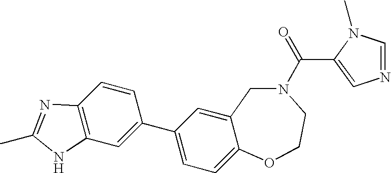 | 7-(2-methyl-1H-benzimidazol-6- yl)-4-[(1-methyl-1H-imidazol-5- yl)carbonyl]-2,3,4,5-tetrahydro- 1,4-benzoxazepine |
| 792 | 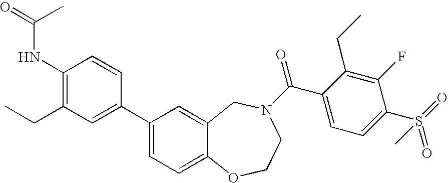 | N-[2-ethyl-4-(4-{[2-ethyl-3- fluoro-4- (methylsulfonyl)phenyl]carbonyl}- 2,3,4,5-tetrahydro-1,4- benzoxazepin-7- yl)phenyl]acetamide |
| 793 | 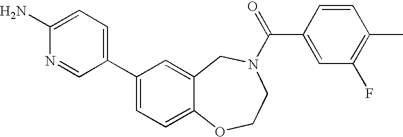 | 5-{4-[(3-fluoro-4- methylphenyl)carbonyl]-2,3,4,5- tetrahydro-1,4-benzoxazepin-7- yl}pyridin-2-amine |
| 794 | 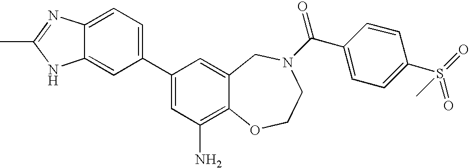 | 7-(2-methyl-1H-benzimidazol-6- yl)-4-{[4- (methylsulfony])phenyl]carbonyl}- 2,3,4,5-tetrahydro-1,4- benzoxazepin-9-amine |
| 795 | 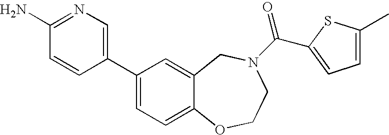 | 5-{4-[(5-methyl-2- thienyl)carbonyl]-2,3,4,5- tetrahydro-1,4-benzoxazepin-7- yl}pyridin-2-amine |
| 796 | 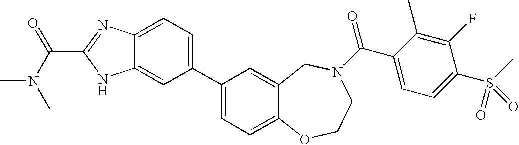 | 6-(4-{[3-fluoro-2-methyl-4- (methylsulfonyl)phenyl]carbonyl}- 2,3,4,5-tetrahydro-1,4- benzoxazepin-7-yl)-N,N- dimethyl-1H-benzimidazole-2- carboxamide |
| 797 | 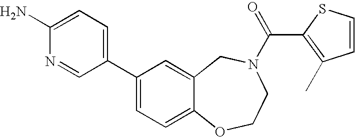 | 5-{4-[(3-methyl-2- thienyl)carbonyl]-2,3,4,5- tetrahydro-1,4-benzoxazepin-7- yl}pyridin-2-amine |
| 798 | 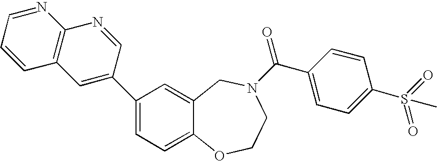 | 4-{[4- (methylsulfonyl)phenyl]carbonyl}- 7-(1,8-naphthyridin-3-yl)- 2,3,4,5-tetrahydro-1,4- benzoxazepine |
| 799 | 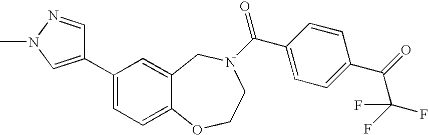 | 2,2,2-trifluoro-1-(4-{[7-(1- methyl-1H-pyrazol-4-yl)-2,3- dihydro-1,4-benzoxazepin- 4(5H- yl]carbonyl}-phenyl)ethanone |
| 800 | 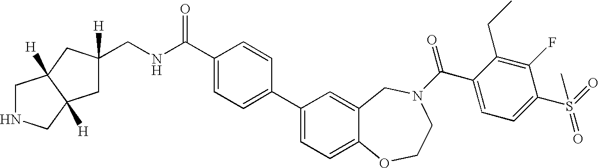 | 4-(4-{[2-ethyl-3-fluoro-4-(methylsulfonyl)phenyl]carbonyl}-2,3,4,5-tetrahydro-1,4-benzoxazepin-7-yl)-N- [(3aR,5r,6aS)-octahydrocyclopenta[c]pyrrol-5-ylmethyl]benzamide |
| 801 | 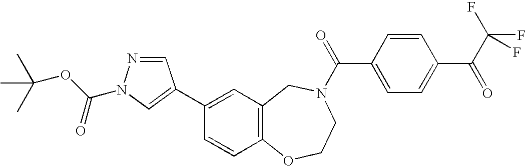 | 1,1-dimethylethyl 4-(4-{[4- (trifluoroacetyl)phenyl]carbonyl}- 2,3,4,5-tetrahydro-1,4- benzoxazepin-7-yl)-1H- pyrazole-1-carboxylate |
| 802 | 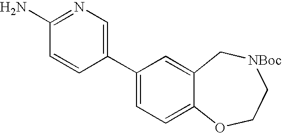 | 1,1-dimethylethyl 7-(6- aminopyridin-3-yl)-2,3-dihydro- 1,4-benzoxazepine-4(5H)- carboxylate |
| 803 | 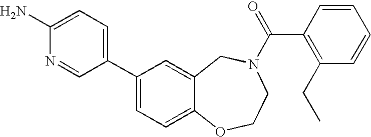 | 5-{4-[(2-ethylphenyl)carbonyl]- 2,3,4,5-tetrahydro-1,4- benzoxazepin-7-yl}pyridin-2- amine |
| 804 | 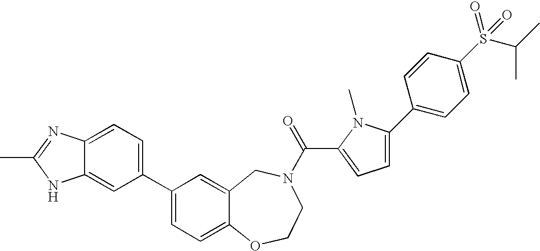 | 7-(2-methyl-1H-benzimidazol-6- yl)-4-[(1-methyl-5-{4-[(1- methylethyl)sulfonyl]phenyl}- 1H-pyrrol-2-yl)carbonyl]- 2,3,4,5-tetrahydro-1,4- benzoxazepine |
| 805 | 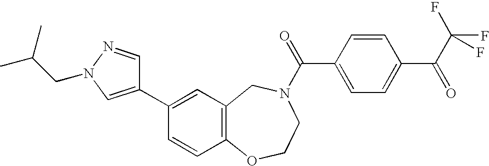 | 2,2,2-trifluoro-1-[4-({7-[1-(2- methylpropyl)-1H-pyrazol-4-yl]- 2,3-dihydro-1,4-benzoxazepin- 4(5H)- yl}carbonyl)phenyl]ethanone |
| 806 | 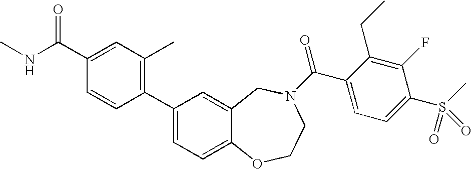 | 4-(4-{[2-ethyl-3-fluoro-4- (methylsulfonyl)phenyl]carbonyl}- 2,3,4,5-tetrahydro-1,4- benzoxazepin-7-yl)-N,3- dimethylbenzamide |
| 807 | 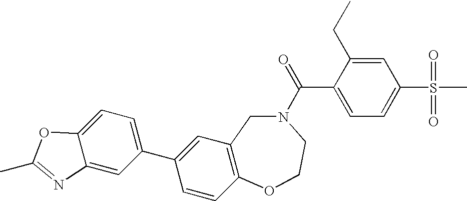 | 4-{[2-ethyl-4- (methylsulfonyl)phenyl]carbonyl}- 7-(2-methyl-1,3-benzoxazol-5- yl)-2,3,4,5-tetrahydro-1,4- benzoxazepine |
| 808 | 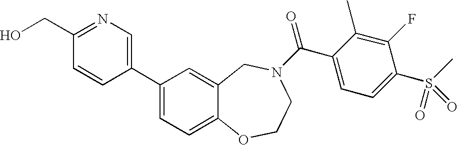 | [5-(4-{[3-fluoro-2-methyl-4- (methylsulfonyl)phenyl]carbonyl}- 2,3,4,5-tetrahydro-1,4- benzoxazepin-7-yl)pyridin-2- yl]methanol |
| 809 | 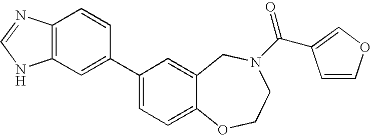 | 7-(1H-benzimidazol-6-yl)-4- (furan-3-ylcarbonyl)-2,3,4,5- tetrahydro-1,4-benzoxazepine |
| 810 | 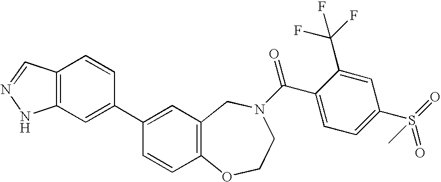 | 7-(1H-indazol-6-yl)-4-{[4- (methylsulfonyl)-2- (trifluoromethyl)phenyl]carbonyl}- 2,3,4,5-tetrahydro-1,4- benzoxazepine |
| 811 | 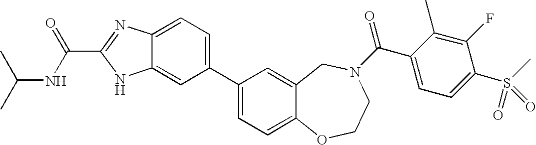 | 6-(4-{[3-fluoro-2-methyl-4- (methylsulfonyl)phenyl]carbonyl}- 2,3,4,5-tetrahydro-1,4- benzoxazepin-7-yl)-N-(1- methylethyl)-1H-benzimidazole- 2-carboxamide |
| 812 | 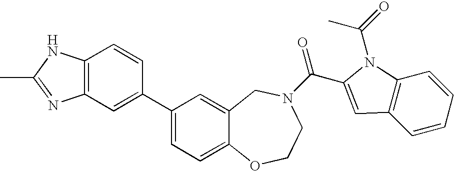 | 4-[(1-acetyl-1H-indol-2- yl)carbonyl]-7-(2-methyl-1H- benzimidazol-5-yl)-2,3,4,5- tetrahydro-1,4-benzoxazepine |
| 813 | 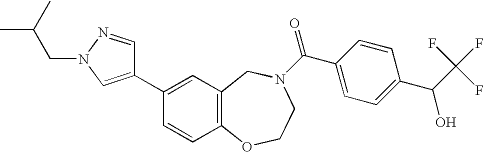 | 2,2,2-trifluoro-1-[4-({7-[1-(2- methylpropyl)-1H-pyrazol-4-yl]- 2,3-dihydro-1,4-benzoxazepin- 4(5H- yl}carbonyl)phenyl]ethanol |
| 814 | 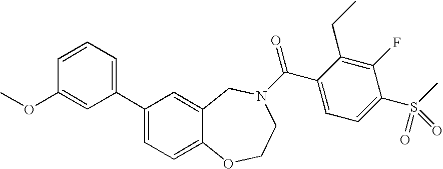 | 4-{[2-ethyl-3-fluoro-4- (methylsulfonyl)phenyl]carbonyl}- 7-[3-(methyloxy)phenyl]- 2,3,4,5-tetrahydro-1,4- benzoxazepine |
| 815 | 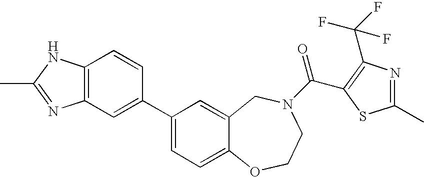 | 7-(2-methyl-1H-benzimidazol-5- yl)-4-{[2-methyl-4- (trifluoromethyl)-1,3-thiazol-5- yl]carbonyl}-2,3,4,5-tetrahydro- 1,4-benzoxazepine |
| 816 | 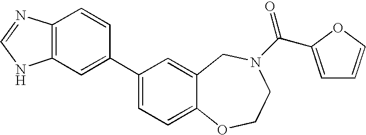 | 7-(1H-benzimidazol-6-yl)-4- (furan-2-ylcarbonyl)-2,3,4,5- tetrahydro-1,4-benzoxazepine |
| 817 | 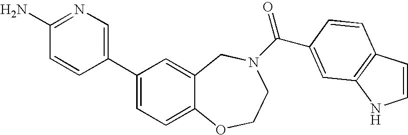 | 5-[4-(1H-indol-6-ylcarbonyl)- 2,3,4,5-tetrahydro-1,4- benzoxazepin-7-yl]pyridin-2- amine |
| 818 | 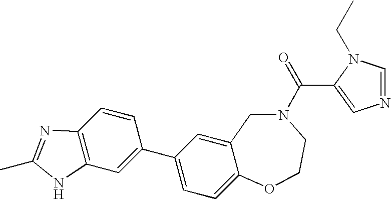 | 4-[(1-ethyl-1H-imidazol-5- yl)carbonyl]-7-(2-methyl-1H- benzimidazol-6-yl)-2,3,4,5- tetrahydro-1,4-benzoxazepine |
| 819 | 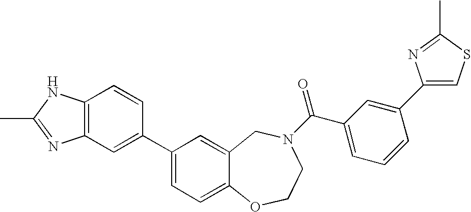 | 7-(2-methyl-1H-benzimidazol-5- yl)-4-{[3-(2-methyl-1,3-thiazol- 4-yl)phenyl]carbonyl}-2,3,4,5- tetrahydro-1,4-benzoxazepine |
| 820 | 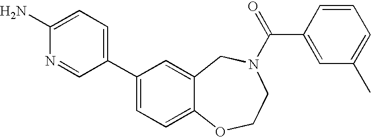 | 5-{4-[(3- methylphenyl)carbonyl]-2,3,4,5- tetrahydro-1,4-benzoxazepin-7- yl}pyridin-2-amine |
| 821 | 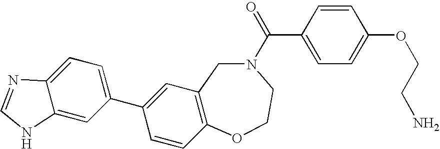 | 2-[(4-{[7-(1H-benzimidazol-6- yl)-2,3-dihydro-1,4- benzoxazepin-4(5H)- yl]carbonyl}phenyl)oxy] ethanamine |
| 822 | 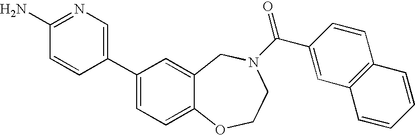 | 5-[4-(naphthalen-2-ylcarbonyl)- 2,3,4,5-tetrahydro-1,4- benzoxazepin-7-yl]pyridin-2- amine |
| 823 | 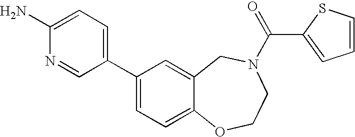 | 5-[4-(2-thienylcarbonyl)-2,3,4,5- tetrahydro-1,4-benzoxazepin-7- yl]pyridin-2-amine |
| 824 | 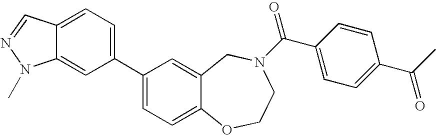 | 1-(4-{[7-(1-methyl-1H-indazol- 6-yl)-2,3-dihydro-1,4- benzoxazepin-4(5H)- yl]carbonyl}phenyl)ethanone |
| 825 | 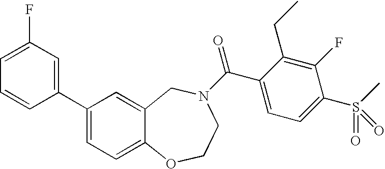 | 4-{[2-ethyl-3-fluoro-4- (methylsulfonyl)phenyl]carbonyl}- 7-(3-fluorophenyl)-2,3,4,5- tetrahydro-1,4-benzoxazepine |
| 826 | 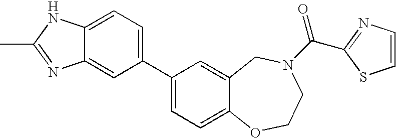 | 7-(2-methyl-1H-benzimidazol-5- yl)-4-(1,3-thiazol-2-ylcarbonyl)- 2,3,4,5-tetrahydro-1,4- benzoxazepine |
| 827 | 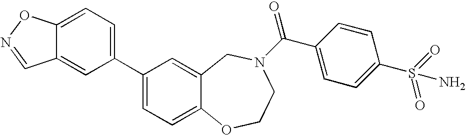 | 4-{[7-(1,2-benzisoxazol-5-yl)- 2,3-dihydro-1,4-benzoxazepin- 4(5H)- yl]carbonyl}benzenesulfonamide |
| 828 | 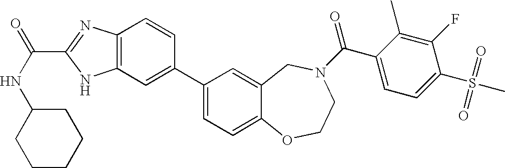 | N-cyclohexyl-6-(4-{[3-fluoro-2- methyl-4- (methylsulfonyl)phenyl]carbonyl}- 2,3,4,5-tetrahydro-1,4- benzoxazepin-7-yl)-1H- benzimidazole-2-carboxamide |
| 829 | 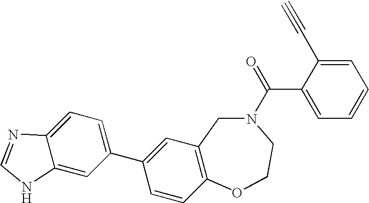 | 7-(1H-benzimidazol-6-yl)-4-[(2- ethynylphenyl)carbonyl]-2,3,4,5- tetrahydro-1,4-benzoxazepine |
| 830 | 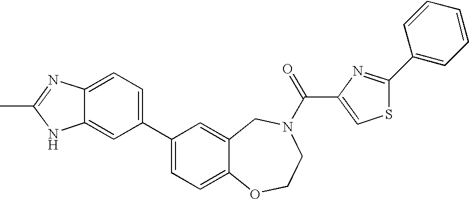 | 7-(2-methyl-1H-benzimidazol-6- yl)-4-[(2-phenyl-1,3-thiazol-4- yl)carbonyl]-2,3,4,5-tetrahydro- 1,4-benzoxazepine |
| 831 | 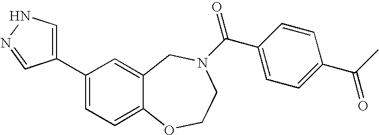 | 1-(4-{[7-(1H-pyrazol-4-yl)-2,3- dihydro-1,4-benzoxazepin- 4(5H)- yl]carbonyl}phenyl)ethanone |
| 832 | 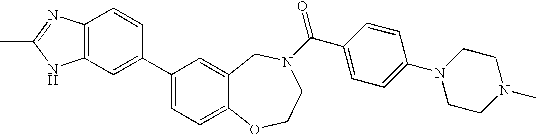 | 7-(2-methyl-1H-benzimidazol-6- yl)-4-{[4-(4-methylpiperazin-1- yl)phenyl]carbonyl}-2,3,4,5- tetrahydro-1,4-benzoxazepine |
| 833 | 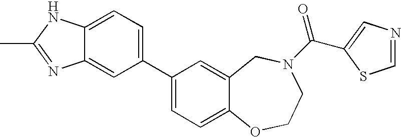 | 7-(2-methyl-1H-benzimidazol-5- yl)-4-(1,3-thiazol-5-ylcarbonyl)- 2,3,4,5-tetrahydro-1,4- benzoxazepine |
| 834 | 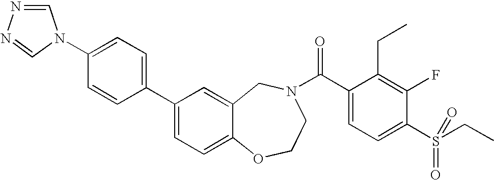 | 4-{[2-ethyl-4-(ethylsulfonyl)-3- fluorophenyl]carbonyl}-7-[4- (4H-1,2,4-triazol-4-yl)phenyl]- 2,3,4,5-tetrahydro-1,4- benzoxazepine |
| 835 | 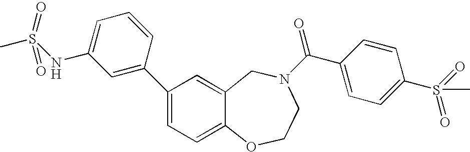 | N-[3-(4-{[4- (methylsulfonyl)phenyl]carbonyl}- 2,3,4,5-tetrahydro-1,4- benzoxazepin-7- yl)phenyl]methanesulfonamide |
| 836 | 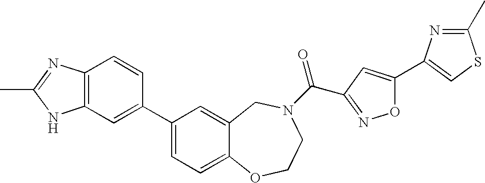 | 7-(2-methyl-1H-benzimidazol-6- yl)-4-{[5-(2-methyl-1,3-thiazol- 4-yl)isoxazol-3-yl]carbonyl}- 2,3,4,5-tetrahydro-1,4- benzoxazepine |
| 837 | 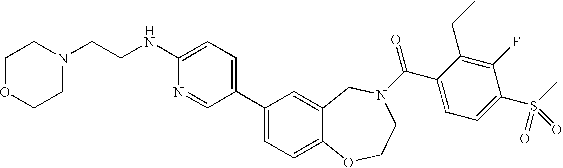 | 5-(4-{[2-ethyl-3-fluoro-4- (methylsulfonyl)phenyl]carbonyl}- 2,3,4,5-tetrahydro-1,4- benzoxazepin-7-yl)-N-(2- morpholin-4-ylethyl)pyridin-2- amine |
| 838 | 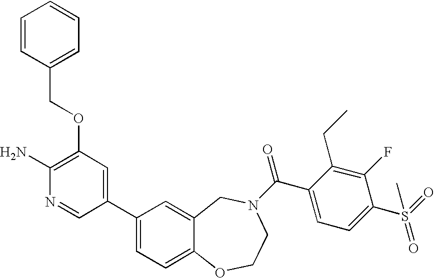 | 5-(4-{[2-ethyl-3-fluoro-4- (methylsulfonyl)phenyl]carbonyl}- 2,3,4,5-tetrahydro-1,4- benzoxazepin-7-yl)-3- [(phenylmethyl)oxy]pyridin-2- amine |
| 839 | 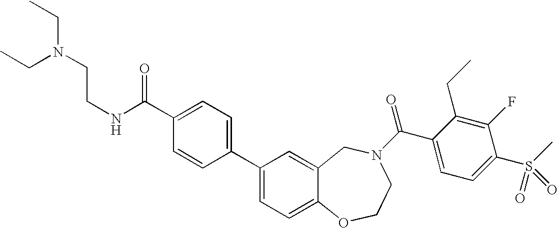 | N-(diethylamino)ethyl]-4-(4- {[2-ethyl-3-fluoro-4- (methylsulfonyl)phenyl]carbonyl}- 2,3,4,5-tetrahydro-1,4- benzoxazepin-7-yl)benzamide |
| 840 | 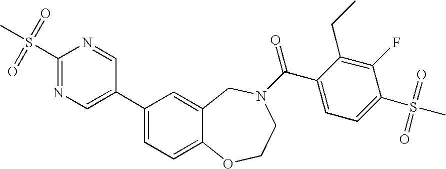 | 4-{[2-ethyl-3-fluoro-4- (methylsulfonyl)phenyl]carbonyl}-7-[2-(methylsulfonyl)pyrimidin- 5-yl]-2,3,4,5-tetrahydro-1,4- benzoxazepine |
| 841 | 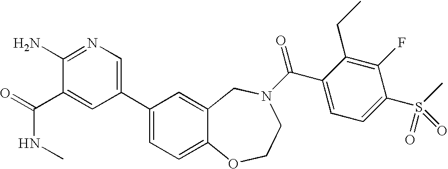 | 2-amino-5-(4-{[2-ethyl-3-fluoro-4- (methylsulfony])phenyl]carbonyl}- 2,3,4,5-tetrahydro-1,4- benzoxazepin-7-yl)-N- methylpyridine-3-carboxamide |
| 842 | 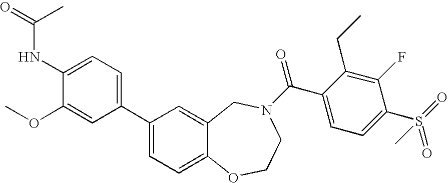 | N-[4-(4-{[2-ethyl-3-fluoro-4- (methylsulfony])phenyl]carbonyl}- 2,3,4,5-tetrahydro-1,4- benzoxazepin-7-yl)-2- (methyloxy)phenyl]acetamide |
| 843 | 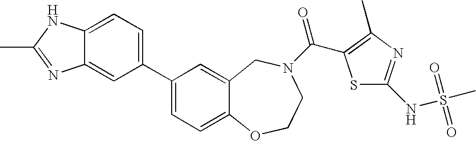 | N-(4-methyl-5-{[7-(2-methyl- 1H-benzimidazol-5-yl)-2,3- dihydro-1,4-benzoxazepin- 4(5H)-yl]carbonyl}-1,3-thiazol- 2-yl)methanesulfonamide |
| 844 | 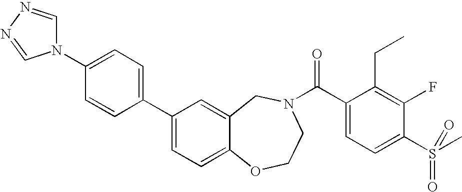 | 4-{[2-ethyl-3-fluoro-4- (methylsulfonyl)phenyl]carbonyl}- 7-[4-(4H-1,2,4-triazol-4- yl)phenyl]-2,3,4,5-tetrahydro- 1,4-benzoxazepine |
| 845 | 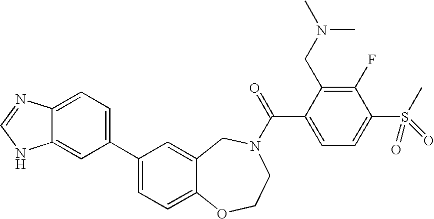 | 1-[6-{[7-(1H-benzimidazol-6- yl)-2,3-dihydro-1,4- benzoxazepin-4(5H)- yl]carbonyl}-2-fluoro-3- (methylsulfonyl)phenyl]-N,N- dimethylmethanamine |
| 846 | 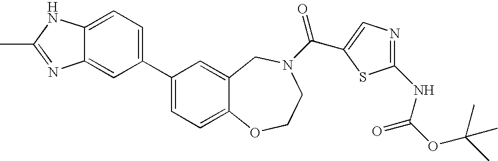 | 1,1-dimethylethyl (5-{[7-(2- methyl-1H-benzimidazol-5-yl)- 2,3-dihydro-1,4-benzoxazepin- 4(5H)-yl]carbonyl}-1,3-thiazol- 2-yl)carbamate |
| 847 | 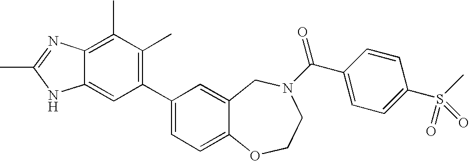 | 4-{[4- (methylsulfonyl)phenyl]carbonyl}- 7-(2,4,5-trimethyl-1H- benzimidazol-6-yl)-2,3,4,5- tetrahydro-1,4-benzoxazepine |
| 848 | 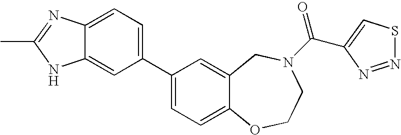 | 7-(2-methyl-1H-benzimidazol-6- yl)-4-(1,2,3-thiadiazol-4- ylcarbonyl)-2,3,4,5-tetrahydro- 1,4-benzoxazepine |
| 849 | 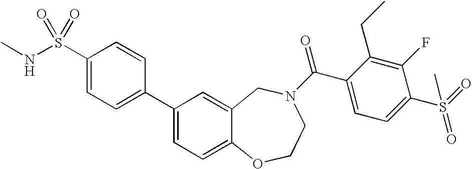 | 4-(4-{[2-ethyl-3-fluoro-4- (methylsulfonyl)phenyl]carbonyl}- 2,3,4,5-tetrahydro-1,4- benzoxazepin-7-yl)-N- methylbenzenesulfonamide |
| 850 | 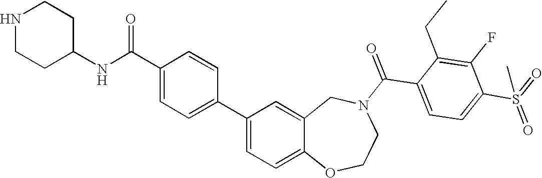 | 4-(4-{[2-ethyl-3-fluoro-4- (methylsulfonyl)phenyl]carbonyl}- 2,3,4,5-tetrahydro-1,4- benzoxazepin-7-yl)-N-piperidin- 4-ylbenzamide |
| 851 | 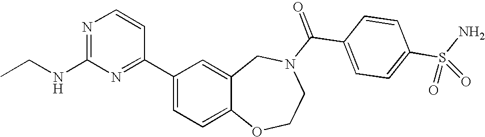 | 4-({7-[2-(ethylamino)pyrimidin- 4-yl]-2,3-dihydro-1,4- benzoxazepin-4(5H)- yl}carbonyl)benzenesulfonamide |
| 852 | 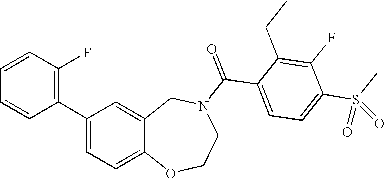 | 4-{[2-ethyl-3-fluoro-4- (methylsulfonyl)phenyl]carbonyl}- 7-(2-fluorophenyl)-2,3,4,5- tetrahydro-1,4-benzoxazepine |
| 853 | 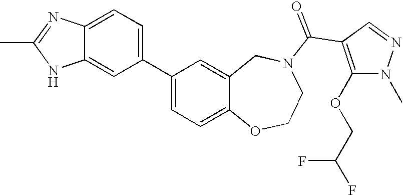 | 4-({5-[(2,2-difluoroethyl)oxy]-1- methyl-1H-pyrazol-4- yl}carbonyl)-7-(2-methyl-1H- benzimidazol-6-yl)-2,3,4,5- tetrahydro-1,4-benzoxazepine |
| 854 | 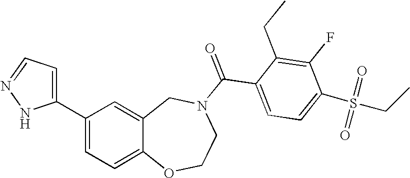 | 4-{[2-ethyl-4-(ethylsulfonyl)-3- fluorophenyl]carbonyl}-7-(1H- pyrazol-5-yl)-2,3,4,5-tetrahydro- 1,4-benzoxazepine |
| 855 | 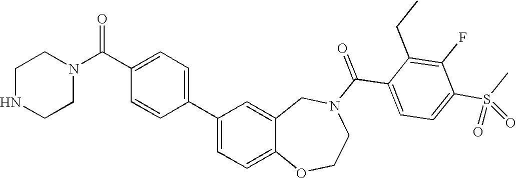 | 4-{[2-ethyl-3-fluoro-4- (methylsulfonyl)phenyl]carbonyl}- 7-[4-(piperazin-1- ylcarbonyl)phenyl]-2,3,4,5- tetrahydro-1,4-benzoxazepine |
| 856 | 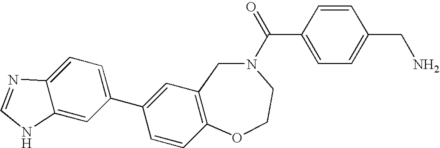 | 1-(4-{[7-(1H-benzimidazol-6- yl)-2,3-dihydro-1,4- benzoxazepin-4(5H)- yl]carbonyl}phenyl)methanamine |
| 857 | 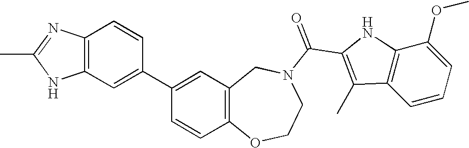 | 7-(2-methyl-1H-benzimidazol-6- yl)-4-{[3-methyl-7-(methyloxy)- 1H-indol-2-yl]carbonyl}-2,3,4,5- tetrahydro-1,4-benzoxazepine |
| 858 | 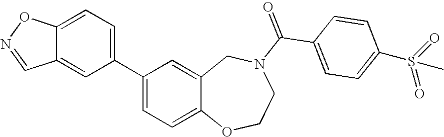 | 7-(1,2-benzisoxazol-5-yl)-4-{[4- (methylsulfonyl)phenyl]carbonyl}- 2,3,4,5-tetrahydro-1,4- benzoxazepine |
| 859 | 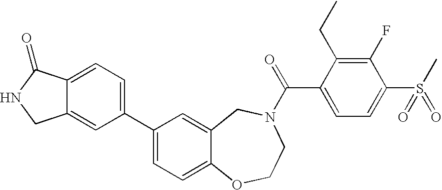 | 5-(4-{[2-ethyl-3-fluoro-4- (methylsulfonyl)phenyl]carbonyl}- 2,3,4,5-tetrahydro-1,4- benzoxazepin-7-yl)-2,3-dihydro- 1H-isoindol-1-one |
| 860 | 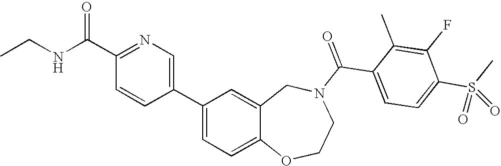 | N-ethyl-5-(4-{[3-fluoro-2- methyl-4- (methylsulfonyl)phenyl]carbonyl}- 2,3,4,5-tetrahydro-1,4- benzoxazepin-7-yl)pyridine-2- carboxamide |
| 861 | 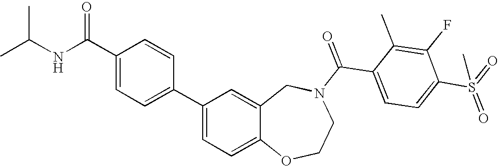 | 4-(4-{[3-fluoro-2-methyl-4- (methylsulfonyl)phenyl]carbonyl}- 2,3,4,5-tetrahydro-1,4- benzoxazepin-7-yl)-N-(1- methylethyl)benzamide |
| 862 | 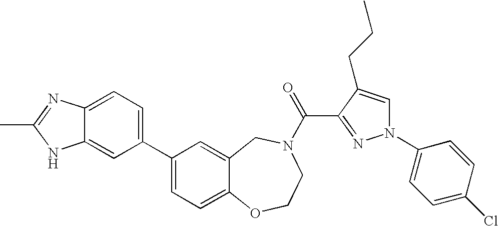 | 4-{[1-(4-chlorophenyl)-4- propyl-1H-pyrazol-3- yl]carbonyl}-7-(2-methyl-1H- benzimidazol-6-yl)-2,3,4,5- tetrahydro-1,4-benzoxazepine |
| 863 | 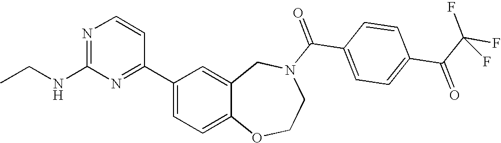 | 1-[4-({7-[2- (ethylamino)pyrimidin-4-yl]-2,3- dihydro-1,4-benzoxazepin- 4(5H)-yl}carbonyl)phenyl]- 2,2,2-trifluoroethanone |
| 864 | 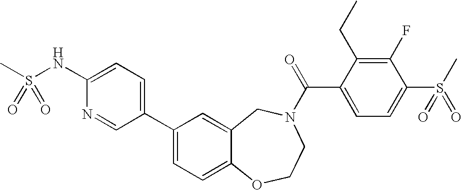 | N-[5-(4-{[2-ethyl-3-fluoro-4- (methylsulfonyl)phenyl]carbonyl}- 2,3,4,5-tetrahydro-1,4- benzoxazepin-7-yl)pyridin-2- yl]methanesulfonamide |
| 865 | 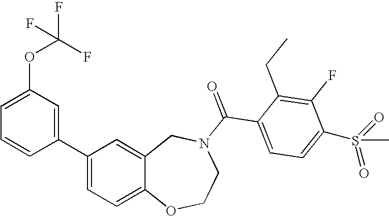 | 4-{[2-ethyl-3-fluoro-4- (methylsulfonyl)phenyl]carbonyl}- 7-{3- [(trifluoromethyl)oxy]phenyl}- 2,3,4,5-tetrahydro-1,4- benzoxazepine |
| 866 | 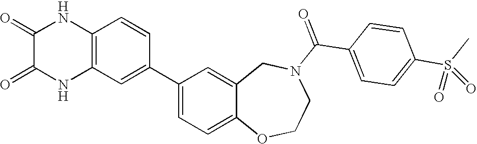 | 6-(4-{[4- (methylsulfonyl)phenyl]carbonyl}- 2,3,4,5-tetrahydro-1,4- benzoxazepin-7-yl)-1,4- dihydroquinoxaline-2,3-dione |
| 867 | 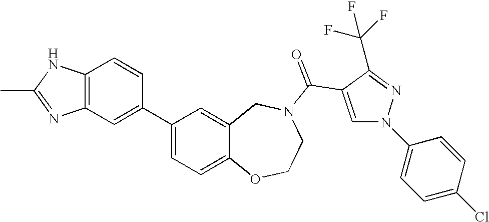 | 4-{[1-(4-chlorophenyl)-3- (trifluoromethyl)-1H-pyrazol-4- yl]carbonyl}-7-(2-methyl-1H- benzimidazol-5-yl)-2,3,4,5- tetrahydro-1,4-benzoxazepine |
| 868 | 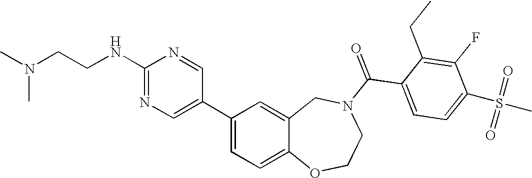 | N′-[5-(4-{[2-ethyl-3-fluoro-4- (methylsulfonyl)phenyl]carbonyl}- 2,3,4,5-tetrahydro-1,4- benzoxazepin-7-yl)pyrimidin-2- yl]-N,N-dimethylethane-1,2- diamine |
| 869 | 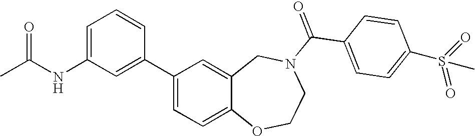 | N-[3-(4-{[4- (methylsulfonyl)phenyl]carbonyl}- 2,3,4,5-tetrahydro-1,4- benzoxazepin-7- yl)phenyl]acetamide |
| 870 | 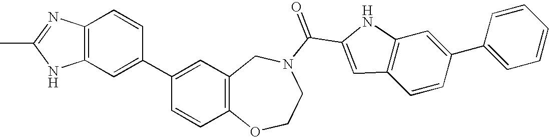 | 7-(2-methyl-1H-benzimidazol-6- yl)-4-[(6-phenyl-1H-indol-2- yl)carbonyl]-2,3,4,5-tetrahydro- 1,4-benzoxazepine |
| 871 | 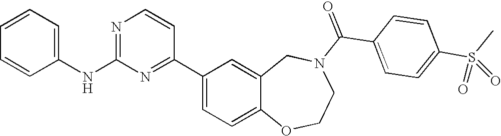 | 4-(4-{[4- (methylsulfonyl)phenyl]carbonyl}- 2,3,4,5-tetrahydro-1,4- benzoxazepin-7-yl)-N- phenylpyrimidin-2-amine |
| 872 | 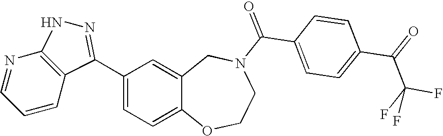 | 2,2,2-trifluoro-1-(4-{[7-(1H-pyrazolo[3,4-b]pyridin-3-yl)-2,3-dihydro-1,4-benzoxazepin-4(5H)- yl]carbonyl}phenyl)ethanone |
| 873 | 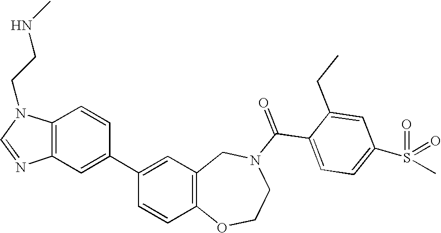 | 2-[5-(4-{[2-ethyl-4- (methylsulfonyl)phenyl]carbonyl}- 2,3,4,5-tetrahydro-1,4- benzoxazepin-7-yl)-1H- benzimidazol-1-yl]-N- methylethanamine |
| 874 | 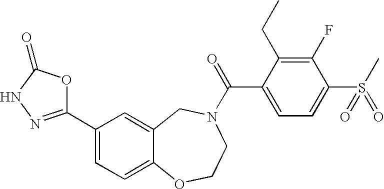 | 5-(4-{[2-ethyl-3-fluoro-4- (methylsulfonyl)phenyl]carbonyl}- 2,3,4,5-tetrahydro-1,4- benzoxazcpin-7-yl)-1,3,4- oxadiazol-2(3H)-one |
| 875 | 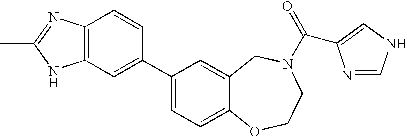 | 4-(1H-imidazol-4-ylcarbonyl)-7- (2-methyl-1H-benzimidazol-6- yl)-2,3,4,5-tetrahydro-1,4- benzoxazepine |
| 876 | 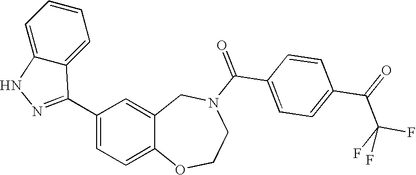 | 2,2,2-trifluoro-1-(4-{[7-(1H- indazol-3-yl)-2,3-dihydro-1,4- benzoxazepin-4(5H)- yl]carbonyl}phenyl)ethanone |
| 877 | 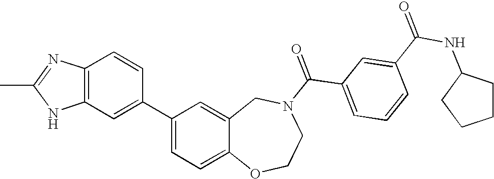 | N-cyclopentyl-3-{[7-(2-methyl- 1H-benzimidazol-6-yl)-2,3- dihydro-1,4-benzoxazepin- 4(5H)-yl]carbonyl}benzamide |
| 878 | 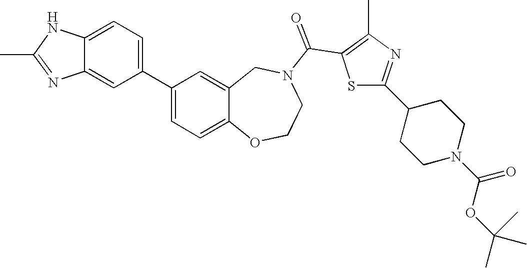 | 1,1-dimethylethyl 4-(4-methyl- 5-{[7-(2-methyl-1H- benzimidazol-5-yl)-2,3-dihydro- 1,4-benzoxazepin-4(5H)- yl]carbonyl}-1,3-thiazol-2- yl)piperidine-1-carboxylate |
| 879 | 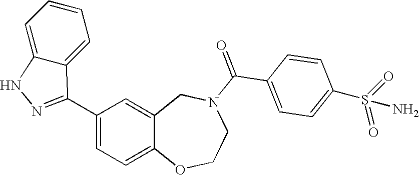 | 4-{[7-(1H-indazol-3-yl)-2,3- dihydro-1,4-benzoxazepin- 4(5H)- yl]carbonyl}benzenesulfonamide |
| 880 | 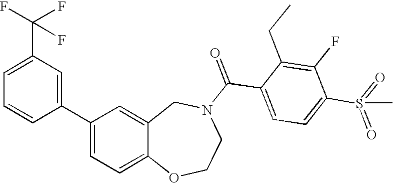 | 4-{[2-ethyl-3-fluoro-4- (methylsulfonyl)phenyl]carbonyl}- 7-[3-(trifluoromethyl)phenyl]- 2,3,4,5-tetrahydro-1,4- benzoxazepine |
| 881 | 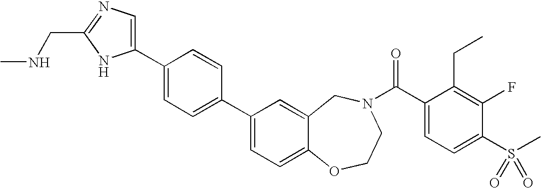 | 1-{5-[4-(4-{[2-ethyl-3-fluoro-4- (methylsulfonyl)phenyl]carbonyl}- 2,3,4,5-tetrahydro-1,4- benzoxazepin-7-yl)phenyl]-1H- imidazol-2-yl}-N- methylmethanamine |
| 882 | 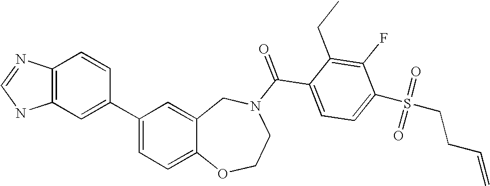 | 7-(1H-benzimidazol-6-yl)-4-{[4- (but-3-en-1-ylsulfonyl)-2-ethyl- 3-fluorophenyl]carbonyl}- 2,3,4,5-tetrahydro-1,4- benzoxazepine |
| 883 | 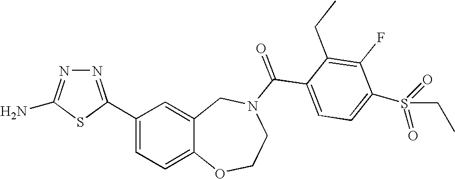 | 5-(4-{[2-ethyl-4-(ethylsulfonyl)- 3-fluorophenyl]carbonyl}- 2,3,4,5-tetrahydro-1,4- benzoxazepin-7-yl)-1,3,4- thiadiazol-2-amine |
| 884 | 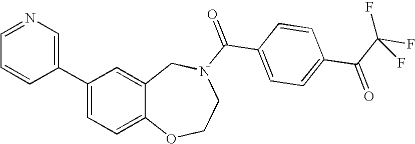 | 2,2,2-trifluoro-1-{4-[(7-pyridin- 3-yl-2,3-dihydro-1,4- benzoxazepin-4(5H)- yl)carbonyl]phenyl}ethanone |
| 885 | 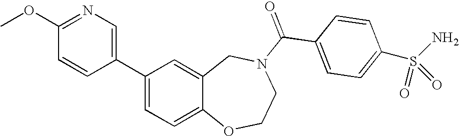 | 4-({7-[6-(methyloxy)pyridin-3- yl]-2,3-dihydro-1,4- benzoxazepin-4(5H)- yl}carbonyl)benzenesulfonamide |
| 886 | 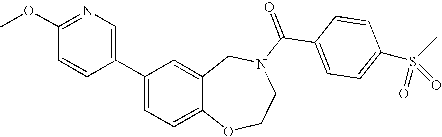 | 7-[6-(methyloxy)pyridin-3-yl]-4- {[4- (methylsulfonyl)phenyl]carbonyl}- 2,3,4,5-tetrahydro-1,4- benzoxazepine |
| 887 | 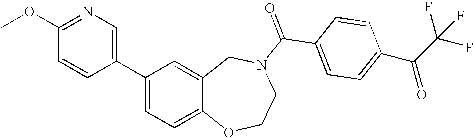 | 2,2,2-trifluoro-1-[4-({7-[6- (methyloxy)pyridin-3-yl]-2,3- dihydro-1,4-benzoxazepin- 4(5H)- yl}carbonyl)phenyl]ethanone |
| 888 | 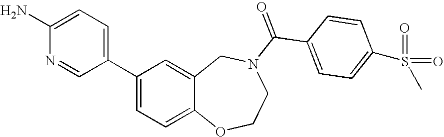 | 5-(4-{[4- (methylsulfonyl)phenyl]carbonyl}- 2,3,4,5-tetrahydro-1,4- benzoxazepin-7-yl)pyridin-2- amine |
| 889 | 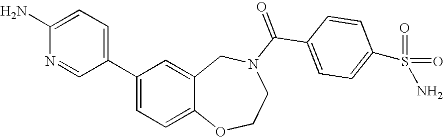 | 4-{[7-(6-aminopyridin-3-yl)-2,3- dihydro-1,4-benzoxazepin- 4(5H)- yl]carbonyl}benzenesulfonamide |
| 890 | 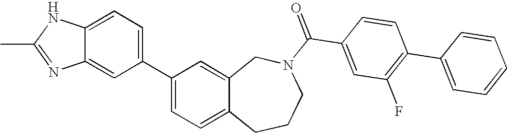 | 4-[(2-fluorobiphenyl-4- yl)carbonyl]-7-(2-methyl-1H- benzimidazol-5-yl)-2,3,4,5- tetrahydro-1,4-benzoxazepine |
| 891 | 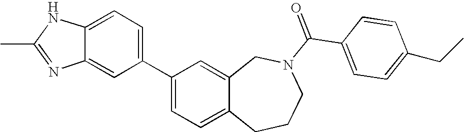 | 4-[(4-ethylphenyl)carbonyl]-7- (2-methyl-1H-benzimidazol-5- yl)-2,3,4,5-tetrahydro-1,4- benzoxazepine |
| 892 | 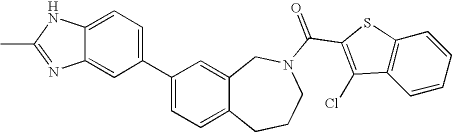 | 4-[(3-chloro-1-benzothien-2- yl)carbonyl]-7-(2-methyl-1H- benzimidazol-5-yl)-2,3,4,5- tetrahydro-1,4-benzoxazepine |
| 893 | 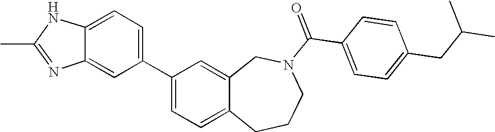 | 7-(2-methyl-1H-benzimidazol-5- yl)-4-{[4-(2- methylpropyl)phenyl]carbonyl}- 2,3,4,5-tetrahydro-1,4- benzoxazepine |
| 894 | 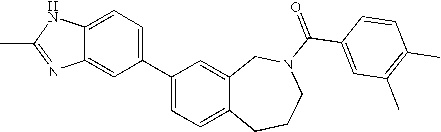 | 4-[(3,4-dimethylphenyl)carbonyl]-7-(2-methyl-1H-benzimidazol-5-yl)-2,3,4,5-tetrahydro-1,4-benzoxazepine |
| 895 | 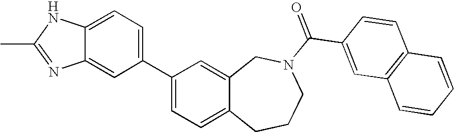 | 7-(2-methyl-1H-benzimidazol-5- yl)-4-(naphthalen-2-ylcarbonyl)- 2,3,4,5-tetrahydro-1,4- benzoxazepine |
| 896 | 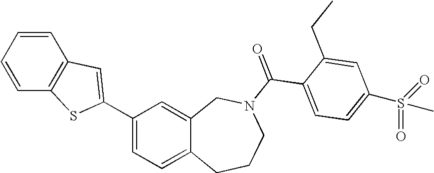 | 7-(1-benzothien-2-yl)-4-{[2- ethyl-4- (methylsulfonyl)phenyl]carbonyl}- 2,3,4,5-tetrahydro-1,4- benzoxazepine |
| 897 | 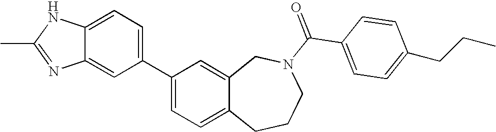 | 7-(2-methyl-1H-benzimidazol-5- yl)-4-[(4- propylphenyl)carbonyl]-2,3,4,5- tetrahydro-1,4-benzoxazepine |
| 898 | 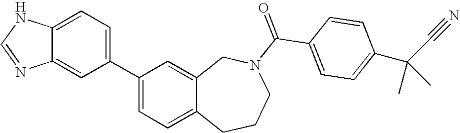 | 2-(4-{[7-(1H-benzimidazol-6- yl)-2,3-dihydro-1,4- benzoxazepin-4(5H)- yl]carbonyl}phenyl)-2-methylpropanenitrile |
| 899 | 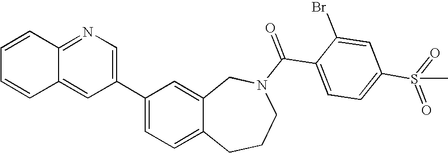 | 4-{[2-bromo-4- (methylsulfonyl)phenyl]carbonyl}- 7-quinolin-3-yl-2,3,4,5- tetrahydro-1,4-benzoxazepine |
| 900 | 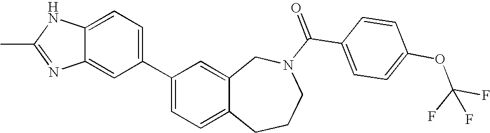 | 7-(2-methyl-1H-benzimidazol-5- yl)-4-({4- [(trifluoromethyl)oxy]phenyl} carbonyl)-2,3,4,5-tetrahydro-1,4- benzoxazepine |
| 901 | 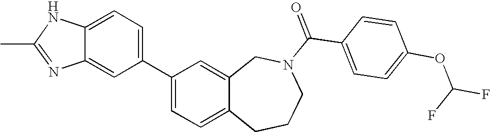 | 4-({4- [(difluoromethyl)oxy]phenyl} carbonyl)-7-(2-methyl-1H- benzimidazol-5-yl)-2,3,4,5- tetrahydro-1,4-benzoxazepine |
| 902 | 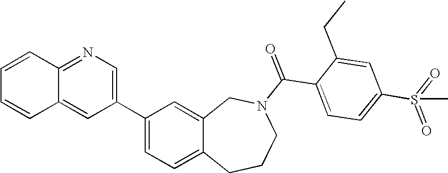 | 4-{[2-ethyl-4- (methylsulfonyl)phenyl]carbonyl}- 7-quinolin-3-yl-2,3,4,5- tetrahydro-1,4-benzoxazepine |
| 903 | 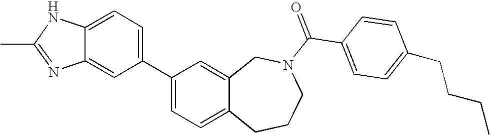 | 4-[(4-butylphenyl)carbonyl]-7- (2-methyl-1H-benzimidazol-5- yl)-2,3,4,5-tetrahydro-1,4- benzoxazepine |
| 904 | 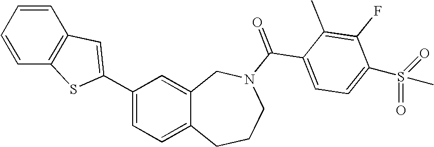 | 7-(1-benzothien-2-yl)-4-{[3- fluoro-2-methyl-4- (methylsulfonyl)phenyl]carbonyl}- 2,3,4,5-tetrahydro-1,4- benzoxazepine |
| 905 | 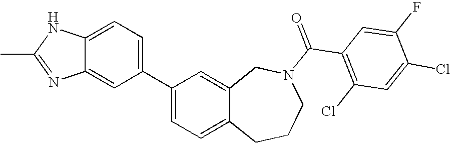 | 4-[(2,4-dichloro-5- fluorophenyl)carbonyl]-7-(2- methyl-1H-benzimidazol-5-yl)- 2,3,4,5-tetrahydro-1,4- benzoxazepine |
| 906 | 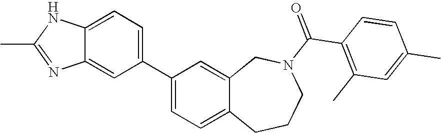 | 4-[(2,4- dimethylphenyl)carbonyl]-7-(2- methyl-1H-benzimidazol-5-yl)- 2,3,4,5-tetrahydro-1,4- benzoxazepine |
| 907 | 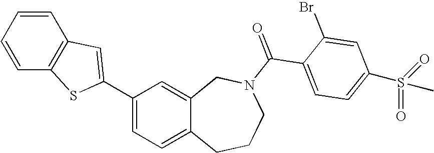 | 7-(1-benzothien-2-yl)-4-{[2- bromo-4- (methylsulfonyl)phenyl]carbonyl}- 2,3,4,5-tetrahydro-1,4- benzoxazepine |
| 908 | 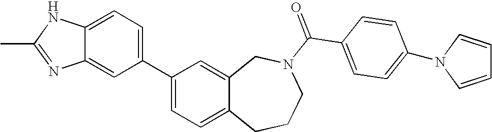 | 7-(2-methyl-1H-benzimidazol-5- yl)-4-{[4-(1H-pyrrol-1- yl)phenyl]carbonyl}-2,3,4,5- tetrahydro-1,4-benzoxazepine |
| 909 | 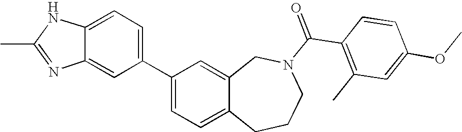 | 7-(2-methyl-1H-benzimidazol-5- yl)-4-{[2-methyl-4- (methyloxy)phenyl]carbonyl}- 2,3,4,5-tetrahydro-1,4- benzoxazepine |
| 910 | 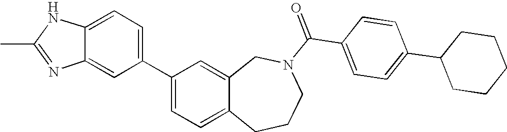 | 4-[(4-cyclohexylphenyl)carbonyl]-7-(2-methyl-1H-benzimidazol-5-yl)-2,3,4,5-tetrahydro-1,4-benzoxazepine |
| 911 | 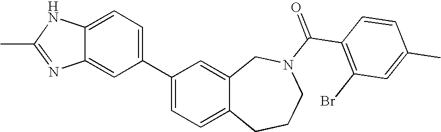 | 4-[(2-bromo-4- methylphenyl)carbonyl]-7-(2- methyl-1H-benzimidazol-5-yl)- 2,3,4,5-tetrahydro-1,4- benzoxazepine |
| 912 | 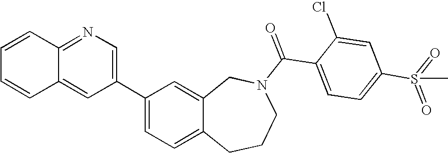 | 4-{[2-chloro-4- (methylsulfonyl)phenyl]carbonyl}- 7-quinolin-3-yl-2,3,4,5- tetrahydro-1,4-benzoxazepine |
| 913 | 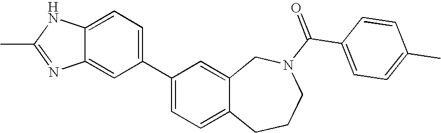 | 7-(2-methyl-1H-benzimidazol-5- yl)-4-[(4- methylphenyl)carbonyl]-2,3,4,5- tetrahydro-1,4-benzoxazepine |
| 914 | 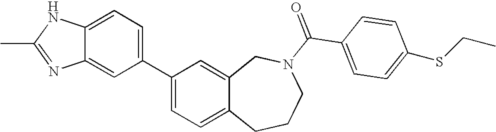 | 4-{[4- (ethylthio)phenyl]carbonyl}-7- (2-methyl-1H-benzimidazol-5- yl)-2,3,4,5-tetrahydro-1,4- benzoxazepine |
| 915 | 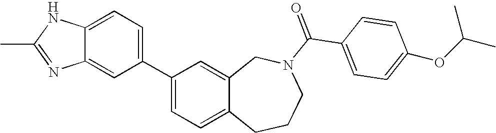 | 7-(2-methyl-1H-benzimidazol-5- yl)-4-({4-[(1-methylethyl)oxy]phenyl} carbonyl)-2,3,4,5-tetrahydro-1,4-benzoxazepine |
| 916 | 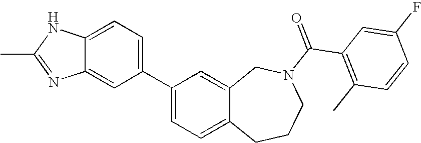 | 4-[(5-fluoro-2- methylphenyl)carbonyl]-7-(2- methyl-1H-benzimidazol-5-yl)- 2,3,4,5-tetrahydro-1,4- benzoxazepine |
| 917 | 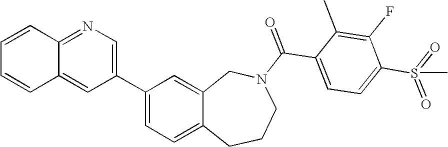 | 4-{[3-fluoro-2-methyl-4- (methylsulfonyl)phenyl]carbonyl}- 7-quinolin-3-yl-2,3,4,5- tetrahydro-1,4-benzoxazepine |
| 918 | 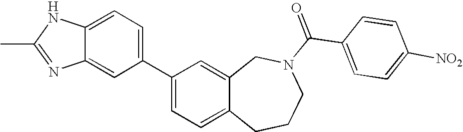 | 7-(2-methyl-1H-benzimidazol-5- yl)-4-[(4-nitrophenyl)carbonyl]- 2,3,4,5-tetrahydro-1,4- benzoxazepine |
| 919 | 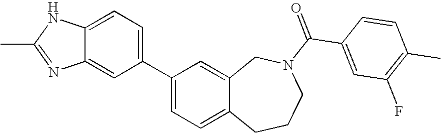 | 4-[(3-fluoro-4- methylphenyl)carbonyl]-7-(2- methyl-1H-benzimidazol-5-yl)- 2,3,4,5-tetrahydro-1,4- benzoxazepine |
| 920 | 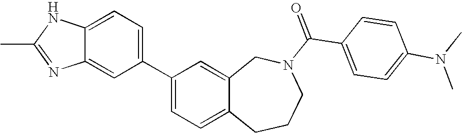 | N,N-dimethyl-4-{[7-(2-methyl- 1H-benzimidazol-5-yl)-2,3- dihydro-1,4-benzoxazepin- 4(5H)-yl]carbonyl}aniline |
| 921 | 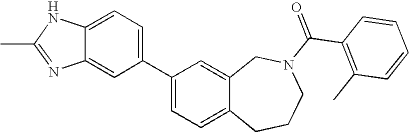 | 7-(2-methyl-1H-benzimidazol-5- yl)-4-[(2- methylphenyl)carbonyl]-2,3,4,5- tetrahydro-1,4-benzoxazepine |
| 922 | 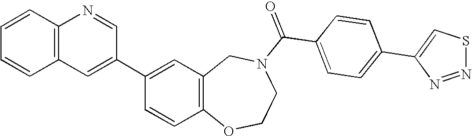 | 7-quinolin-3-yl-4-{[4-(1,2,3- thiadiazol-4- yl)phenyl]carbonyl}-2,3,4,5- tetrahydro-1,4-benzoxazepine |
| 923 | 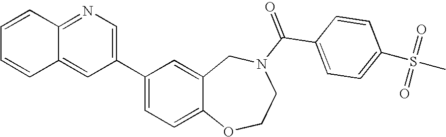 | 4-{[4- (methylsulfonyl)phenyl]carbonyl}- 7-quinolin-3-yl-2,3,4,5- tetrahydro-1,4-benzoxazepine |
| 924 | 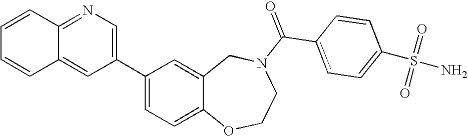 | 4-[(7-quinolin-3-yl-2,3-dihydro- 1,4-benzoxazepin-4(5H)- yl)carbonyl]benzenesulfonamide |
| 925 | 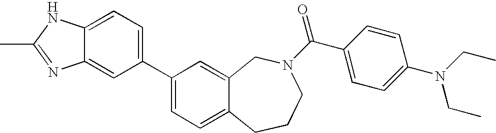 | N,N-diethyl-4-{[7-(2-methyl- 1H-benzimidazol-5-yl)-2,3- dihydro-1,4-benzoxazepin- 4(5H)-yl]carbonyl}aniline |
| 926 | 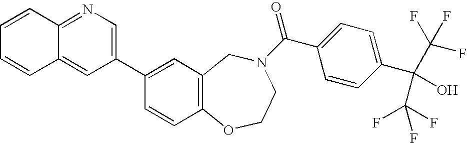 | 1,1,1,3,3,3-hexafluoro-2-{4-[(7- quinolin-3-yl-2,3-dihydro-1,4- benzoxazepin-4(5H)- yl)carbonyl]phenyl}propan-2-ol |
| 927 | 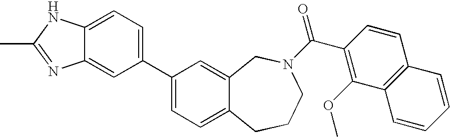 | 7-(2-methyl-1H-benzimidazol-5- yl)-4-{[1- (methyloxy)naphthalen-2-yl]carbonyl}-2,3,4,5-tetrahydro- 1,4-benzoxazepine |
| 928 | 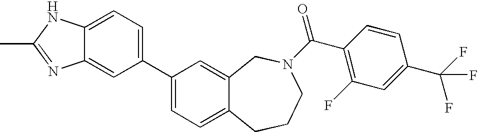 | 4-{[2-fluoro-4-(trifluoromethyl)phenyl]carbonyl}-7-(2-methyl-1H- benzimidazol-5-yl)-2,3,4,5-tetrahydro-1,4-benzoxazepine |
| 929 | 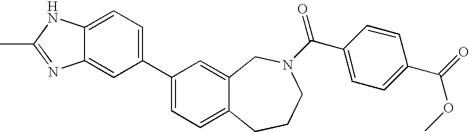 | methyl 4-{[7-(2-methyl-1H- benzimidazol-5-yl)-2,3-dihydro- 1,4-benzoxazepin-4(5H)- yl]carbonyl}benzoate |
| 930 | 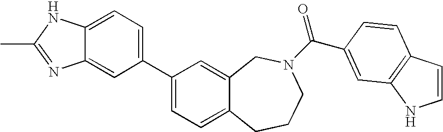 | 4-(1H-indol-6-ylcarbonyl)-7-(2- methyl-1H-benzimidazol-5-yl)- 2,3,4,5-tetrahydro-1,4- benzoxazepine |
| 931 | 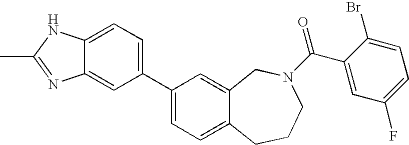 | 4-[(2-bromo-5- fluorophenyl)carbonyl]-7-(2- methyl-1H-benzimidazol-5-yl)- 2,3,4,5-tetrahydro-1,4- benzoxazepine |
| 932 | 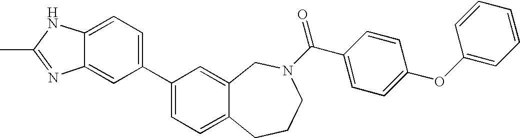 | 7-(2-methyl-1H-benzimidazol-5- yl)-4-{[4- (phenyloxy)phenyl]carbonyl}- 2,3,4,5-tetrahydro-1,4- benzoxazepine |
| 933 | 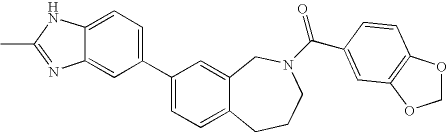 | 4-(1,3-benzodioxol-5- ylcarbonyl)-7-(2-methyl-1H- benzimidazol-5-yl)-2,3,4,5- tetrahydro-1,4-benzoxazepine |
| 934 | 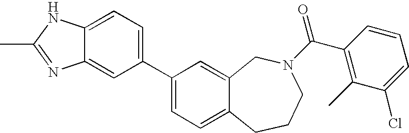 | 4-[(3-chloro-2- methylphenyl)carbonyl]-7-(2- methyl-1H-benzimidazol-5-yl)- 2,3,4,5-tetrahydro-1,4- benzoxazepine |
| 935 | 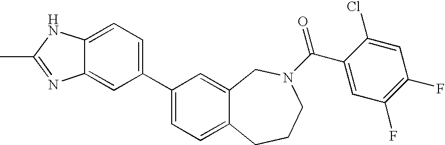 | 4-[(2-chloro-4,5- difluorophenyl)carbonyl]-7-(2- methyl-1H-benzimidazol-5-yl)- 2,3,4,5-tetrahydro-1,4- benzoxazepine |
| 936 | 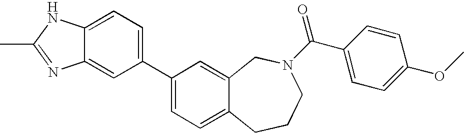 | 7-(2-methyl-1H-benzimidazol-5- yl)-4-{[4-(methyloxy)phenyl]carbonyl}- 2,3,4,5-tetrahydro-1,4- benzoxazepine |
| 937 | 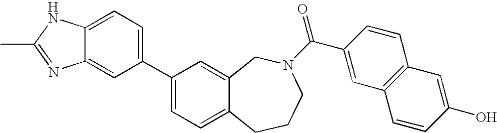 | 6-{[7-(2-methyl-1H- benzimidazol-5-yl)-2,3-dihydro- 1,4-benzoxazepin-4(5H)- yl]carbonyl}naphthalen-2-ol |
| 938 | 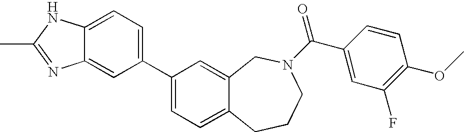 | 4-{[3-fluoro-4- (methyloxy)phenyl]carbonyl}-7- (2-methyl-1H-benzimidazol-5- yl)-2,3,4,5-tetrahydro-1,4- benzoxazepine |
| 939 | 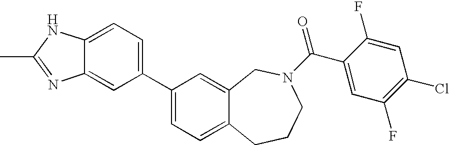 | 4-[(4-chloro-2,5- difluorophenyl)carbonyl]-7-(2- methyl-1H-benzimidazol-5-yl)- 2,3,4,5-tetrahydro-1,4- benzoxazepine |
| 940 | 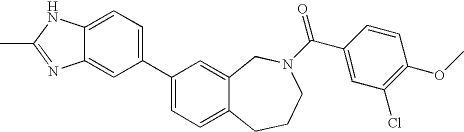 | 4-{[3-chloro-4- (methyloxy)phenyl]carbonyl}-7- (2-methyl-1H-benzimidazol-5- yl)-2,3,4,5-tetrahydro-1,4-benzoxazepine |
| 941 | 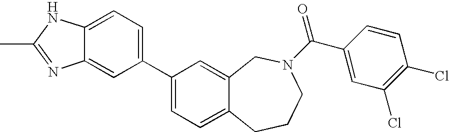 | 4-[(3,4- dichlorophenyl)carbonyl]-7-(2- methyl-1H-benzimidazol-5-yl)- 2,3,4,5-tetrahydro-1,4- benzoxazepine |
| 942 | 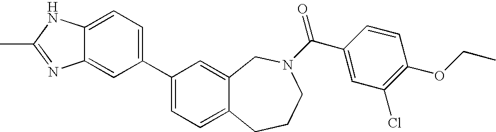 | 4-{[3-chloro-4- (ethyloxy)phenyl]carbonyl}-7- (2-methyl-1H-benzimidazol-5- yl)-2,3,4,5-tetrahydro-1,4-benzoxazepine |
| 943 | 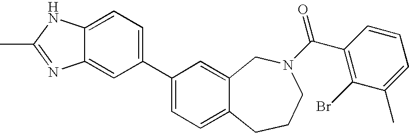 | 4-[(2-bromo-3-methylphenyl)carbonyl]-7-(2-methyl-1H-benzimidazol-5-yl)-2,3,4,5-tetrahydro-1,4-benzoxazepine |
| 944 | 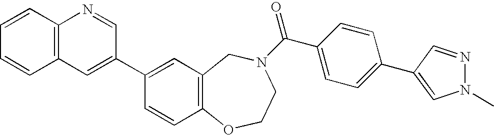 | 4-{[4-(1-methyl-1H-pyrazol-4- yl)phenyl]carbonyl}-7-quinolin- 3-yl-2,3,4,5-tetrahydro-1,4- benzoxazepine |
| 945 | 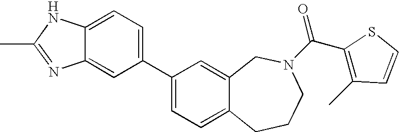 | 7-(2-methyl-1H-benzimidazol-5- yl)-4-[(3-methyl-2- thienyl)carbonyl]-2,3,4,5- tetrahydro-1,4-benzoxazepine |
| 946 | 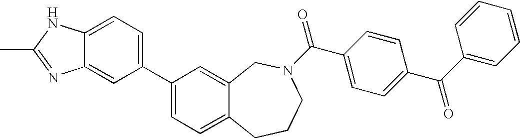 | (4-{[7-(2-methyl-1H- benzimidazol-5-yl)-2,3-dihydro- 1,4-benzoxazepin-4(5H)- yl]carbonyl}phenyl)(phenyl) methanone |
| 947 | 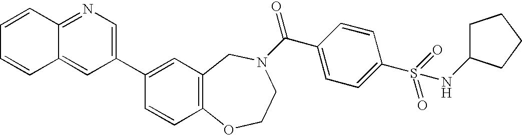 | N-cyclopentyl-4-[(7-quinolin-3- yl-2,3-dihydro-1,4- benzoxazepin-4(5H)- yl)carbonyl]benzenesulfonamide |
| 948 | 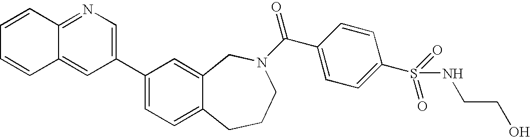 | N-(2-hydroxyethyl)-4-[(7- quinolin-3-yl-2,3-dihydro-1,4- benzoxazepin-4(5H)- yl)carbonyl]benzenesulfonamide |
| 949 | 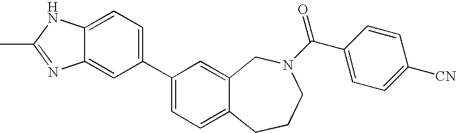 | 4-{[7-(2-methyl-1H- benzimidazol-5-yl)-2,3-dihydro- 1,4-benzoxazepin-4(5H)- yl]carbonyl}benzonitrile |
| 950 | 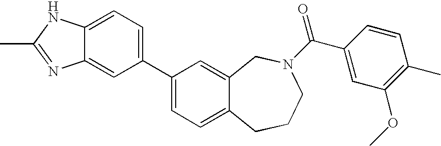 | 7-(2-methyl-1H-benzimidazol-5- yl)-4-{[4-methyl-3- (methyloxy)phenyl]carbonyl}- 2,3,4,5-tetrahydro-1,4- benzoxazepine |
| 951 | 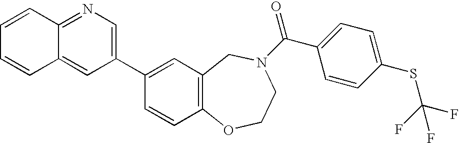 | 7-quinolin-3-yl-4-({4- [(trifluoromethyl)thio]phenyl} carbonyl)-2,3,4,5-tetrahydro-l,4- benzoxazepine |
| 952 | 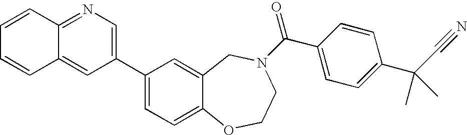 | 2-methyl-2-{4-[(7-quinolin-3-yl- 2,3-dihydro-1,4-benzoxazepin-4(5H)- yl)carbonyl]phenyl}propanenitrile |
| 953 | 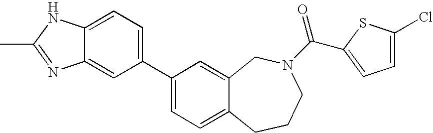 | 4-[(5-chloro-2- thienyl)carbonyl]-7-(2-methyl- 1H-benzimidazol-5-yl)-2,3,4,5- tetrahydro-1,4-benzoxazepine |
| 954 | 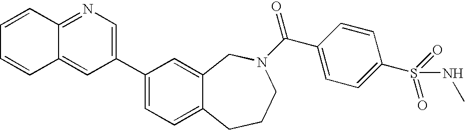 | N-methyl-4-[(7-quinolin-3-yl- 2,3-dihydro-1,4-benzoxazepin- 4(5H)- yl)carbonyl]benzenesulfonamide |
| 955 | 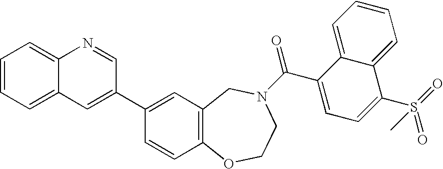 | 4-{[4- (methylsulfonyl)naphthalen-1- yl]carbonyl}-7-quinolin-3-yl- 2,3,4,5-tetrahydro-1,4- benzoxazepine |
| 956 | 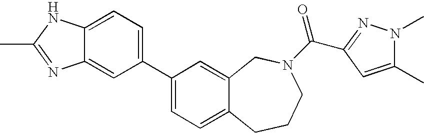 | 4-[(1,5-dimethyl-1H-pyrazol-3- yl)carbonyl]-7-(2-methyl-1H- benzimidazol-5-yl)-2,3,4,5- tetrahydro-1,4-benzoxazepine |
| 957 | 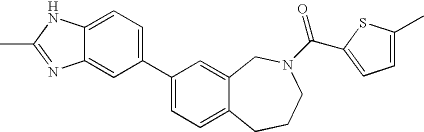 | 7-(2-methyl-1H-benzimidazol-5- yl)-4-[(5-methyl-2- thienyl)carbonyl]-2,3,4,5- tetrahydro-1,4-benzoxazepine |
| 958 | 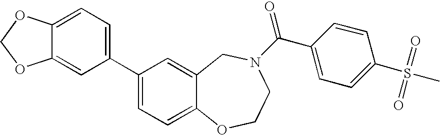 | 7-(1,3-benzodioxol-5-yl)-4-{[4- (methylsulfonyl)phenyl]carbonyl}- 2,3,4,5-tetrahydro-1,4- benzoxazepine |
| 959 | 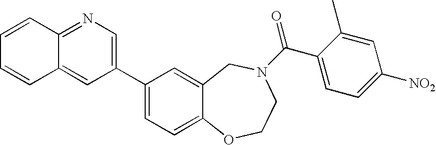 | 4-[(2-methyl-4- nitrophenyl)carbonyl]-7- quinolin-3-yl-2,3,4,5-tetrahydro- 1,4-benzoxazepine |
| 960 | 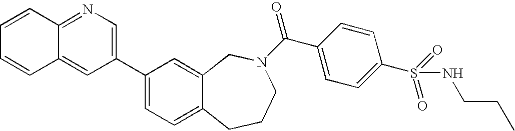 | N-propyl-4-[(7-quinolin-3-yl- 2,3-dihydro-1,4-benzoxazepin- 4(5H)- yl)carbonyl]benzenesulfonamide |
| 961 | 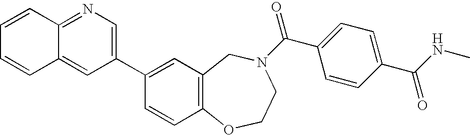 | N-methyl-4-[(7-quinolin-3-yl- 2,3-dihydro-1,4-benzoxazepin- 4(5H)-yl)carbonyl]benzamide |
| 962 | 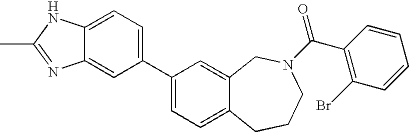 | 4-[(2-bromophenyl)carbonyl]-7- (2-methyl-1H-benzimidazol-5- yl)-2,3,4,5-tetrahydro-1,4- benzoxazepine |
| 963 | 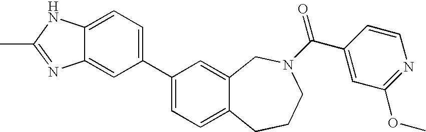 | 7-(2-methyl-1H-benzimidazol-5- yl)-4-{[2-(methyloxy)pyridin-4- yl]carbonyl}-2,3,4,5-tetrahydro- 1,4-benzoxazepine |
| 964 | 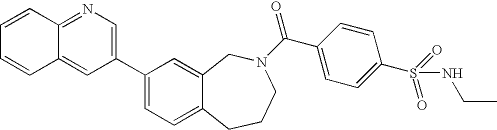 | N-ethyl-4-[(7-quinolin-3-yl-2,3- dihydro-1,4-benzoxazepin- 4(5H)- yl)carbonyl]benzenesulfonamide |
| 965 | 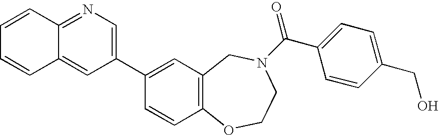 | {4-[(7-quinolin-3-yl-2,3- dihydro-1,4-benzoxazepin- 4(5H)- yl)carbonyl]phenyl}methanol |
| 966 | 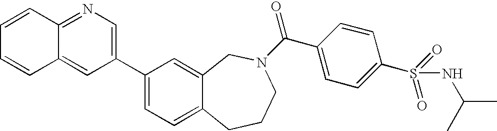 | N-(1-methylethyl)-4-[(7- quinolin-3-yl-2,3-dihydro-1,4- benzoxazepin-4(5H)- yl)carbonyl]benzenesulfonamide |
| 967 | 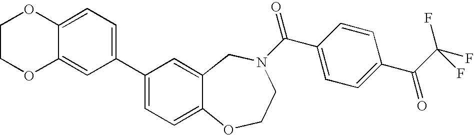 | 1-(4-{[7-(2,3-dihydro-1,4- benzodioxin-6-yl)-2,3-dihydro- 1,4-benzoxazepin-4(5H)- yl]carbonyl}phenyl)-2,2,2- trifluoroethanone |
| 968 | 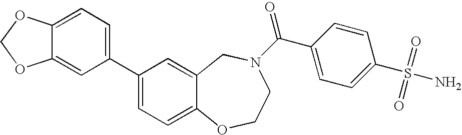 | 4-{[7-(1,3-benzodioxol-5-yl)- 2,3-dihydro-1,4-benzoxazepin- 4(5H)- yl]carbonyl}benzenesulfonamide |
| 969 | 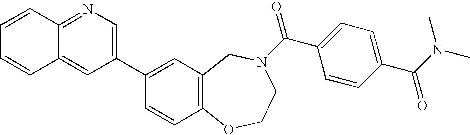 | N,N-dimethyl-4-[(7-quinolin-3- yl-2,3-dihydro-1,4- benzoxazepin-4(5H)- yl)carbonyl]benzamide |
| 970 | 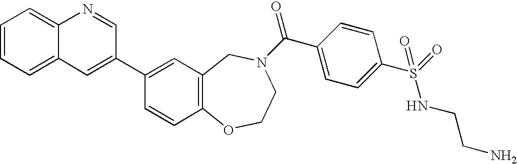 | N-(2-aminoethyl)-4-[(7- quinolin-3-yl-2,3-dihydro-1,4- benzoxazepin-4(5H)- yl)carbonyl]benzenesulfonamide |
| 971 | 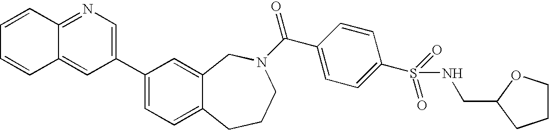 | 4-[(7-quinolin-3-yl-2,3-dihydro- 1,4-benzoxazepin-4(5H)- yl)carbonyl]-N-(tetrahydrofuran- 2-ylmethyl)benzenesulfonamide |
| 972 | 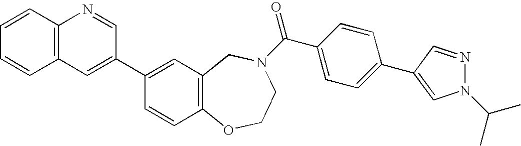 | 4-({4-[1-(1-methylethyl)-1H- pyrazol-4-yl]phenyl}carbonyl)- 7-quinolin-3-yl-2,3,4,5- tetrahydro-1,4-benzoxazepine |
| 973 | 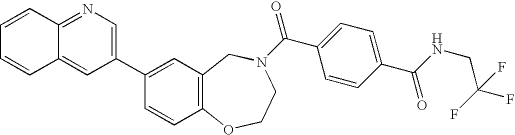 | 4-[(7-quinolin-3-yl-2,3-dihydro- 1,4-benzoxazepin-4(5H)- yl)carbonyl]-N-(2,2,2- trifluoroethyl)benzamide |
| 974 | 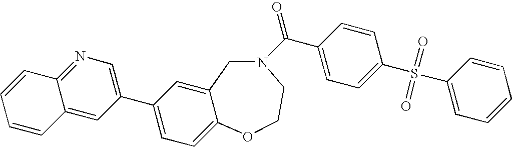 | 4-{[4- (phenylsulfonyl)phenyl]carbonyl}- 7-quinolin-3-yl-2,3,4,5- tetrahydro-1,4-benzoxazepine |
| 975 | 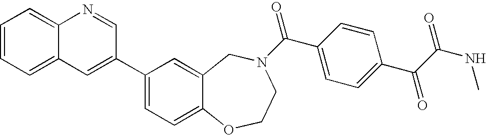 | N-methyl-2-oxo-2-{4-[(7- quinolin-3-yl-2,3-dihydro-1,4- benzoxazepin-4(5H)- yl)carbonyl]phenyl}acetamide |
| 976 | 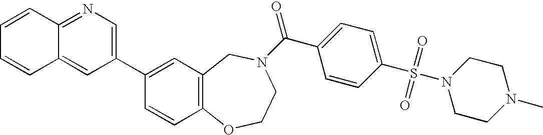 | 4-({4-[(4-methylpiperazin-1- yl)sulfonyl]phenyl}carbonyl)-7- quinolin-3-yl-2,3,4,5-tetrahydro- 1,4-benzoxazepine |
| 977 | 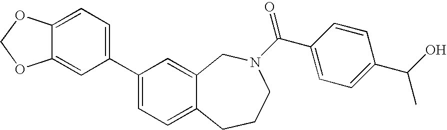 | 1-(4-{[7-(1,3-benzodioxol-5-yl)- 2,3-dihydro-1,4-benzoxazepin- 4(5H)- yl]carbonyl}phenyl)ethanol |
| 978 | 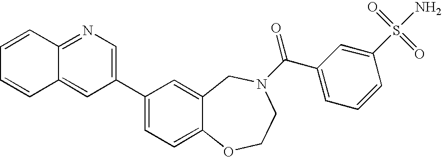 | 3-[(7-quinolin-3-yl-2,3-dihydro- 1,4-benzoxazepin-4(5H)- yl)carbonyl]benzenesulfonamide |
| 979 | 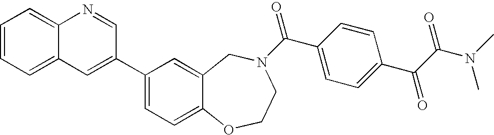 | N,N-dimethyl-2-oxo-2-{4-[(7- quinolin-3-yl-2,3-dihydro-1,4- benzoxazepin-4(5H)- yl)carbonyl]phenyl}acetamide |
| 980 | 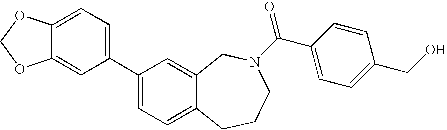 | (4-{[7-(1,3-benzodioxol-5-yl)- 2,3-dihydro-1,4-benzoxazepin- 4(5H)- yl]carbonyl}phenyl)methanol |
| 981 | 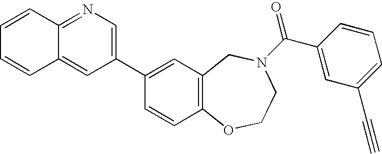 | 4-[(3-ethynylphenyl)carbonyl]- 7-quinolin-3-yl-2,3,4,5- tetrahydro-1,4-benzoxazepine |
| 982 | 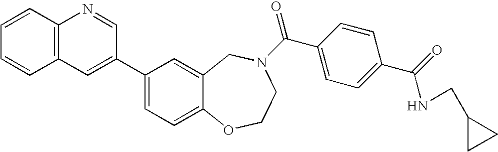 | N-(cyclopropylmethyl)-4-[(7- quinolin-3-yl-2,3-dihydro-1,4- benzoxazepin-4(5H)- yl)carbonyl]benzamide |
| 983 | 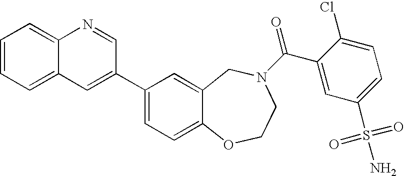 | 4-chloro-3-[(7-quinolin-3-yl-2,3- dihydro-1,4-benzoxazepin- 4(5H)- yl)carbonyl]benzenesulfonamide |
| 984 | 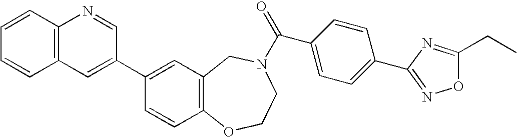 | 4-{[4-(5-ethyl-1,2,4-oxadiazol- 3-yl)phenyl]carbonyl}-7- quinolin-3-yl-2,3,4,5-tetrahydro- 1,4-benzoxazepine |
| 985 | 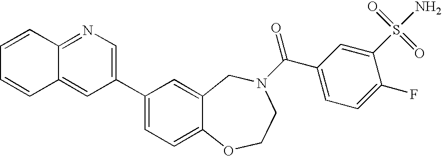 | 2-fluoro-5-[(7-quinolin-3-yl-2,3- dihydro-1,4-benzoxazepin- 4(5H)- yl)carbonyl]benzenesulfonamide |
| 986 | 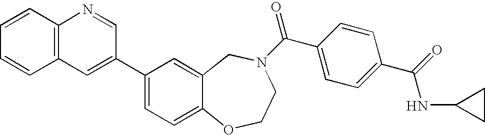 | N-cyclopropyl-4-[(7-quinolin-3- yl-2,3-dihydro-1,4- benzoxazepin-4(5H)- yl)carbonyl]benzamide |
| 987 | 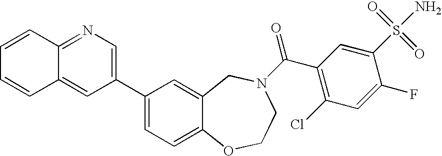 | 4-chloro-2-fluoro-5-[(7- quinolin-3-yl-2,3-dihydro-1,4- benzoxazepin-4(5H)- yl)carbonyl]benzenesulfonamide |
| 988 | 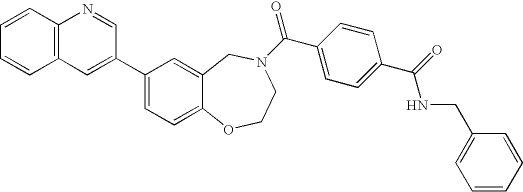 | N-(phenylmethyl)-4-[(7- quinolin-3-yl-2,3-dihydro-1,4- benzoxazepin-4(5H)- yl)carbonyl]benzamide |
| 989 | 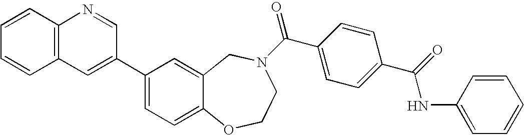 | N-phenyl-4-[(7-quinolin-3-yl- 2,3-dihydro-1,4-benzoxazepin- 4(5H)-yl)carbonyl]benzamide |
| 990 | 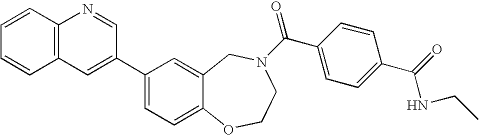 | N-ethyl-4-[(7-quinolin-3-yl-2,3- dihydro-1,4-benzoxazepin- 4(5H)-yl)carbonyl]benzamide |
| 991 | 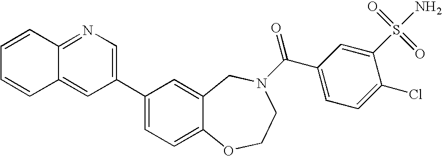 | 2-chloro-5-[(7-quinolin-3-yl-2,3- dihydro-1,4-benzoxazepin- 4(5H)- yl)carbonyl]benzenesulfonamide |
| 992 | 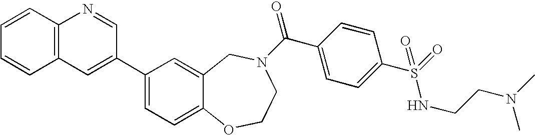 | N-[2-(dimethylamino)ethyl]-4- [(7-quinolin-3-yl-2,3-dihydro- 1,4-benzoxazepin-4(5H)- yl)carbonyl]benzenesulfonamide |
| 993 | 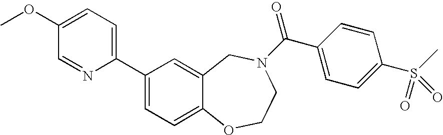 | 7-[5-(methyloxy)pyridin-2-yl]-4- {[4- (methylsulfonyl)phenyl]carbonyl}- 2,3,4,5-tetrahydro-1,4- benzoxazepine |
| 994 | 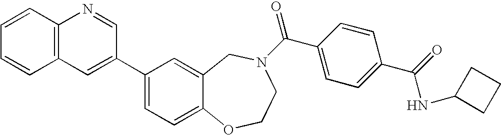 | N-cyclobutyl-4-[(7-quinolin-3- yl-2,3-dihydro-1,4- benzoxazepin-4(5H)- yl)carbonyl]benzamide |
| 995 | 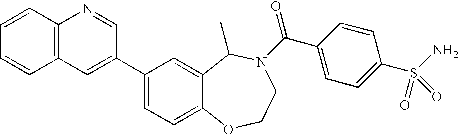 | 4-[(5-methyl-7-quinolin-3-yl- 2,3-dihydro-1,4-benzoxazepin- 4(5H)- yl)carbonyl]benzenesulfonamide |
| 996 | 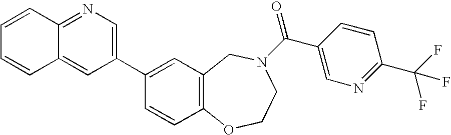 | 7-quinolin-3-yl-4-{[6- (trifluoromethyl)pyridin-3- yl]carbonyl}-2,3,4,5-tetrahydro- 1,4-benzoxazepine |
| 997 | 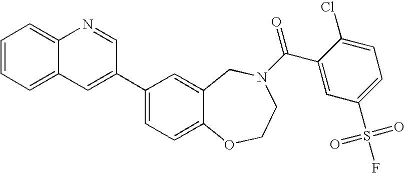 | 4-chloro-3-[(7-quinolin-3-yl-2,3- dihydro-1,4-benzoxazepin- 4(5H)- yl)carbonyl] benzenesulfonylfluoride |
| 998 | 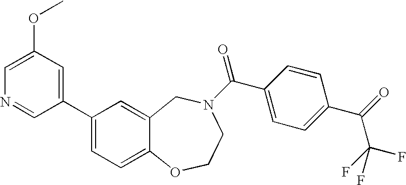 | 2,2,2-trifluoro-1-[4-({7-[5- (methyloxy)pyridin-3-yl]-2,3- dihydro-1,4-benzoxazepin- 4(5H)- yl}carbonyl)phenyl]ethanone |
| 999 | 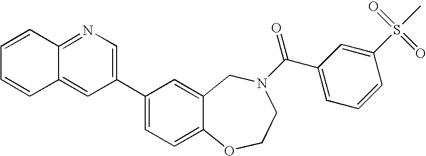 | 4-{[3- (methylsulfonyl)phenyl]carbonyl}- 7-quinolin-3-yl-2,3,4,5- tetrahydro-1,4-benzoxazepine |

### General Administration

In one aspect, the invention provides pharmaceutical compositions comprising an inhibitor of mTOR according to the invention and a pharmaceutically acceptable carrier, excipient, or diluent. In certain other specific embodiments, administration is by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions will include a conventional pharmaceutical carrier or excipient and a compound of the invention as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

### General Synthesis

Compounds of this invention can be made by the synthetic procedures described below. The starting materials and reagents used in preparing these compounds are either available from commercial suppliers such as Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure and over a temperature range from about −78° C. to about 150° C., more specifically from about 0° C. to about 125° C. and more specifically at about room (or ambient) temperature, e.g., about 20° C. Unless otherwise stated (as in the case of an hydrogenation), all reactions are performed under an atmosphere of nitrogen.

Prodrugs can be prepared by techniques known to one skilled in the art. These techniques generally modify appropriate functional groups in a given compound. These modified functional groups regenerate original functional groups by routine manipulation or in vivo. Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of the Invention that may be prepared through the syntheses described herein may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention.

Some of the compounds of the invention contain an active ketone —C(O)CF₃ and may exist in part or in whole as the —C(OH₂)CF₃ form. Regardless of whether the compound is drawn as the —C(O)CF₃ or —C(OH₂)CF₃ form, both are included within the scope of the Invention. Although an individual compound may be drawn as the —C(O)CF₃ form, one of ordinary skill in the art would understand that the compound may exist in part or in whole as the —C(OH₂)CF₃ form and that the ratio of the two forms may vary depending on the compound and the conditions in which it exists.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention. Further, for example, in this application R¹ can be 5-oxo-1H-1,2,4-triazol-3-yl, depicted structurally below:

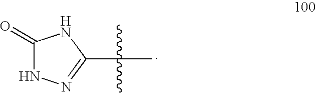

Both 5-oxo-1H-1,2,4-triazol-3-yl and the above structure 1 include, and are equivalent to, 3-hydroxy-4H-1,2,4-triazol-5-yl and its structure 2:

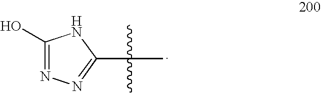

Regardless of which structure or which terminology is used, each tautomer is included within the scope of the Invention.

The present invention also includes N-oxide derivatives and protected derivatives of compounds of the Invention. For example, when compounds of the Invention contain an oxidizable nitrogen atom, the nitrogen atom can be converted to an N-oxide by methods well known in the art. When compounds of the Invention contain groups such as hydroxy, carboxy, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. The protected derivatives of compounds of the Invention can be prepared by methods well known in the art.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The chemistry for the preparation of the compounds of this invention is known to those skilled in the art. In fact, there may be more than one process to prepare the compounds of the invention. The following examples illustrate but do not limit the invention. All references cited herein are incorporated by reference in their entirety.

An intermediate of formula 4 where PG is a nitrogen-protecting group, R^{5a} and R^{5c} are independently hydrogen or alkyl, R^{5h} is hydrogen or halo, R^{5b} is hydrogen, amino, or halo, and R^{5d}, R^{5e}, R^{5f}, and R^{5g} are hydrogen can be prepared according to Scheme 1.

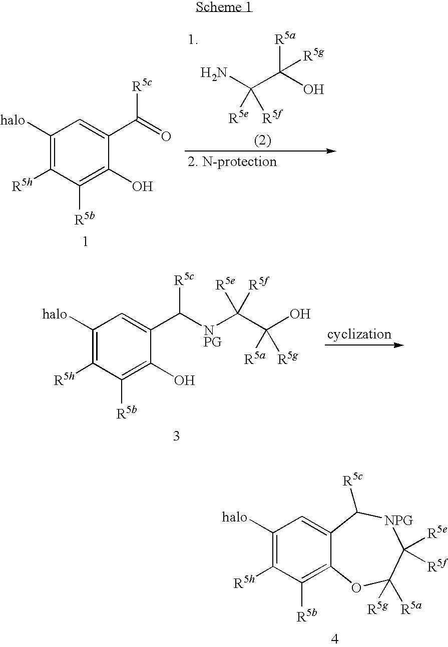

In particular, an intermediate of formula 4a can be prepared according to Scheme 1a.

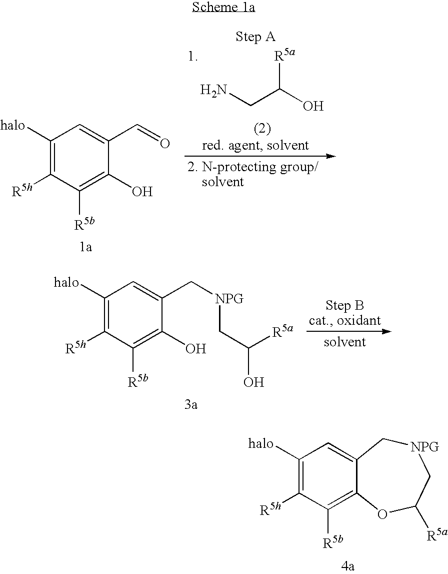

An intermediate of formula 1a is commercially available or can be prepared using methods known to one of ordinary skill in the art. In particular an intermediate of formula 1a where R^{5b} is hydrogen and R^{5h} is hydrogen, bromo, or chloro is commercially available. An intermediate of formula 1a where R^{5h} is hydrogen and R^{5b} is bromo, chloro, iodo, or fluoro is commercially available. An intermediate of formula 1a where R^{5h} is fluoro and R^{5b} is hydrogen can be prepared using procedures described in J. of Med. Chem., 2004, 47(12), 3163-3179. An intermediate of formula 1a where R^{5h} is hydrogen and R^{5b} is amino can be prepared from the corresponding, commercially-available nitro intermediate using procedures known to one of ordinary skill in the art.

An intermediate of formula 2a where R^{5a} is hydrogen or methyl is commercially available. The intermediate of formula 1a is treated with an intermediate of formula 2a in the presence of a reducing agent such as sodium borohydride, in a solvent(s) such as tetrahydrofuran and/or methanol and allowed to react at a temperature of about 40° C. for approximately 4 hours. The solvent is then removed and the reaction is taken up in a solvent(s) such as ethyl acetate and/or saturated sodium bicarbonate. To this suspension a nitrogen-protecting group precursor, such as di-tert-butyl dicarbonate, is added and the mixture is allowed to stir at room temperature overnight to yield an intermediate of formula 3a where PG is a nitrogen-protecting group.

Intermediate 3a is then treated with a catalyst, such as triphenylphosphine, in the presence of a dehydrating agent such as diisopropyl azodicarboxylate, in a solvent such as DCM. The reaction is allowed to proceed at room temperature for approximately 12 hours and the resulting product is optionally purified by column chromatography to yield an intermediate of formula 4a. Alternatively, the intermediate of formula 4a can be prepared by treating the intermediate of formula 3a with Burgess' reagent.

An intermediate of formula 5 where PG is a nitrogen-protecting group, R^{5a} and R^{5c} are independently hydrogen or alkyl, R^{5h} is hydrogen or halo, R^{5b} is hydrogen, amino, or halo, R^{5e}, R^{5f}, and R^{5g} are hydrogen, and R¹ is as defined in the Summary of the Invention for a Compound of Formula I can be prepared according to Scheme 2.

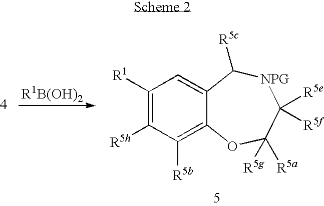

where the intermediate of formula 4 is prepared as described in Scheme 1.

In particular, an intermediate of formula 5a where R^{5a} is hydrogen or alkyl, R^{5h} is hydrogen or halo, R^{5b} is hydrogen, amino, or halo, and R¹ is as defined in the Summary of the Invention for a Compound of Formula I, can be prepared according to Scheme 2a.

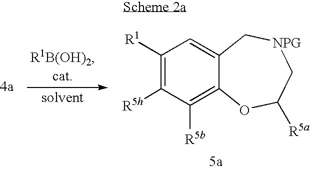

The intermediate of formula 4a, prepared as described in Scheme 1a, is treated with a boronic acid of formula R¹B(OH)₂, which is commercially available or can be prepared using procedures known to one of ordinary skill in the art. The reaction is carried out in the presence of a catalyst such as Pd(dppf)₂Cl₂, a base such as potassium carbonate, and in a solvent such as DME at about 80° C. for about 2 hours. The product can then be purified by chromatography to yield an intermediate of formula 5a.

Alternatively, an intermediate of formula 5, as defined above, can be prepared as described in Scheme 4.

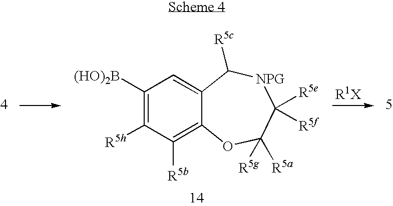

In particular, an intermediate of formula 5b where PG is a nitrogen-protecting group and R¹ is as defined in the Summary of the Invention for a Compound of Formula I can be prepared according to Scheme 4a.

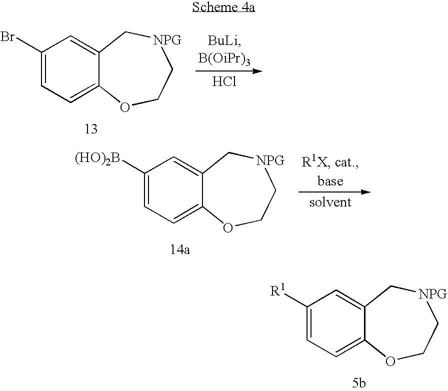

An intermediate of formula 13, where PG is a nitrogen-protecting group, is prepared as described in Scheme 1a. 13 is treated with triisopropylborate in a solvent such as THF at a temperature of about −60° C., followed by dropwise addition of a base such as n-butyllithium in tetrahydrofuran. The reaction was allowed to proceed for about 30 minutes, was treated with an acid such as hydrochloric acid, and allowed to warm to room temperature to yield an intermediate of formula 14a. Intermediate 14a is then treated with an intermediate of formula R¹X (where X is a halide, and which is commercially available or can be prepared using procedures known to one of ordinary skill in the art), in the presence of a base such as potassium carbonate, in the presence of a catalyst such as tetrakis(triphenylphosphine)palladium(0), and in a solvent(s) such as 1,2-dimethoxyethane and/or water. The reaction is allowed to proceed under nitrogen and stirred at reflux for about 3 hours to yield an intermediate of formula 5b.

A Compound of the Invention of Formula I where Z is C(O), R^{5a} and R^{5c} are independently hydrogen or alkyl, R^{5h} is hydrogen or halo, R^{5b} is hydrogen, amino, or halo, R^{5e}, R^{5f}, and R^{5g} are hydrogen, and R¹ and R² are as defined in the Summary of the Invention for a Compound of Formula I can be prepared as described in Scheme 5,

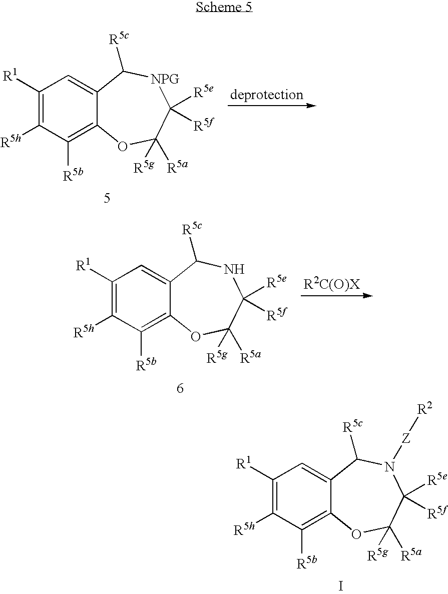

In particular, a Compound of Formula I(q) where Z is C(O), R^{5a} is hydrogen or alkyl, R^{5h} is hydrogen or halo, R^{5b} is hydrogen, amino, or halo, and R¹ and R² are as defined in the Summary of the Invention for a Compound of Formula I can be prepared as described in Scheme 5a.

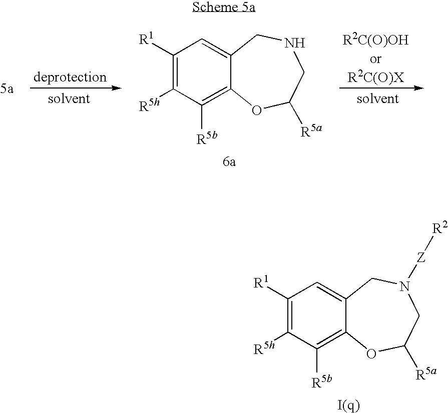

The protecting group on the intermediate of formula 5a is removed. When the protecting group is Boc, it can be removed with HCl to yield an intermediate of formula 6a. The intermediate of formula R²C(O)OH is commercially available or can be prepared using procedures described in Scheme 3a or 3b or procedures known to one of ordinary skill in the art. The intermediate of formula 6a is then treated with R²C(O)OH, in the presence of a coupling agent(s) such as HATU and/or HOBt, in the presence of a base such as Hünig's base, in a solvent such as DMF, at a temperature of about 50° C. Alternatively, the intermediate of formula 6 can be treated with an acid chloride of formula R²C(O)X (where X is halo, and which is commercially available or can be prepared using procedures described in Scheme 3b or known to one of ordinary skill in the art), in the presence of a base such as Hünig's base, and in a solvent such as DMF. The product can be purified by column chromatography to yield an intermediate of Formula I(q).

In particular, a Compound of Formula I(r) where Z is C(O) and R¹ and R² are as defined in the Summary of the Invention for a Compound of Formula I can be prepared according to Scheme 5b.

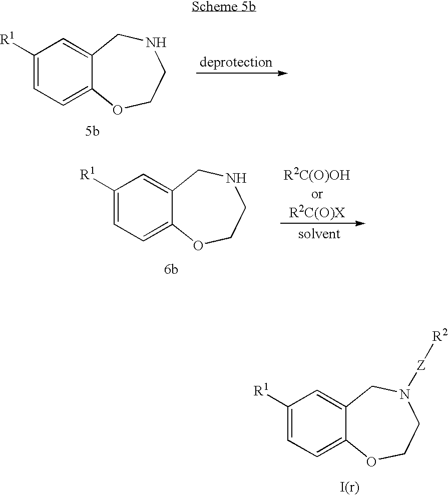

The protecting group on intermediate of formula 5b, prepared as described in Scheme 4a, is removed. When the protecting group is Boc, it can be removed with HCl to yield an intermediate of formula 6b. Intermediate 6b is then treated with an intermediate of R²C(O)OH or R²C(O)X using standard amide coupling conditions to yield a Compound of Formula I(r).

An intermediate of formula 26 where R⁶ is as defined in the Summary of the Invention for a Compound of Formula I (except not where R⁶ is halo) and R^{3c} is as defined in the Summary of the Invention for a Compound of Formula I, is useful in the preparation of Compounds of the Invention and can be prepared according to Scheme 3a.

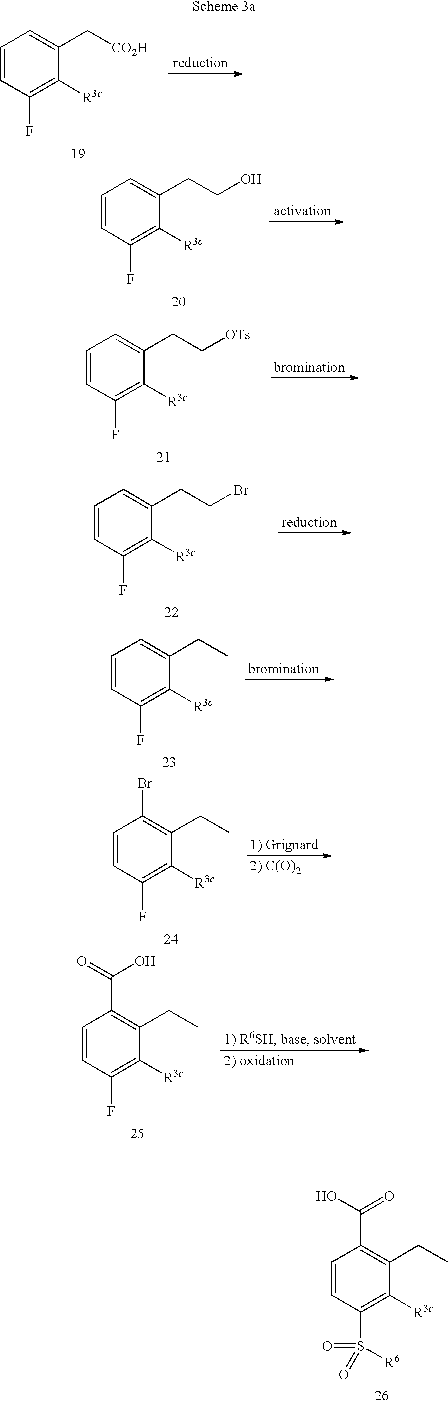

The intermediate of formula 19 is commercially available or can be prepared using procedures known to one of ordinary skill in the art. 19 is treated with a reducing agent, such as BH₃-Me₂S, in a solvent such as THF for about an hour at room temperature to yield an intermediate of formula 20. The intermediate of formula 20 is then treated with an activating agent such as mesyl chloride or tosyl chloride, in the presence of a base such as triethylamine, and in a solvent such as DCM. The reaction is allowed to proceed for about five hours at room temperature to yield an intermediate of formula 21. The intermediate of formula 21 is then treated with a brominating agent such as LiBr in a solvent such as acetone and allowed to reflux for about 3 hours to yield an intermediate of formula 22. Intermediate 22 is then reduced to intermediate 23 in the presence of magnesium and 1,2-dibromoethane in a solvent such as ether. Intermediate 23 is then treated with a brominating agent such as Br₂, in the presence of iron, in a solvent such as chloroform and allowed to react at room temperature for approximately overnight to yield intermediate 24. Intermediate 24 can then be treated with a Grignard reagent such as isopropyl magnesium chloride in a solvent such as THF, followed by treatment with C(O)₂ to yield the intermediate of formula 24. Intermediate 24 is then treated with an intermediate of formula R⁶SH [or NaS(alkyl)] in a solvent such as DMSO. The reaction is quenched with water, worked up, and then treated with oxone in the presence of a base such as NaOH, and in the presence of NaHCO₃, in a solvent such as acetone to yield the intermediate of formula 26.

The intermediate of formula 26 can then be treated with an intermediate of formula 6, 6a, or 6b using conditions described in Scheme 5, 5a, or 5b to yield a Compound of the Invention of Formula I(ff), I(gg), or I(hh)

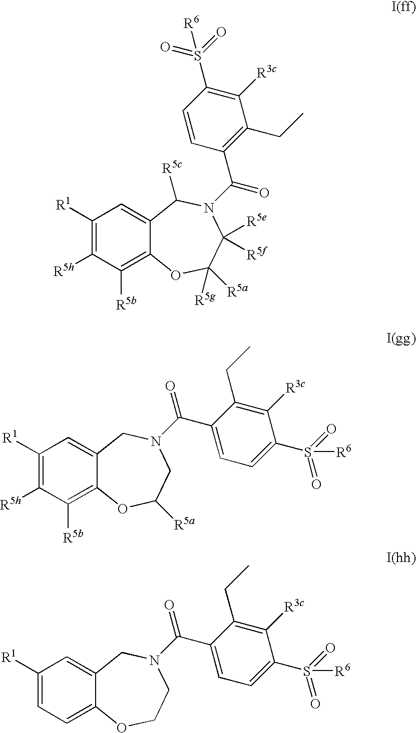

respectively.

An intermediate of formula 12 where R^{3a} is —S(O)₂R⁶, R^{3b} is alkyl, R^{3c} is halo, and R⁶ is alkyl is useful in the preparation of a Compound of Formula I and can be prepared according to Scheme 3b.

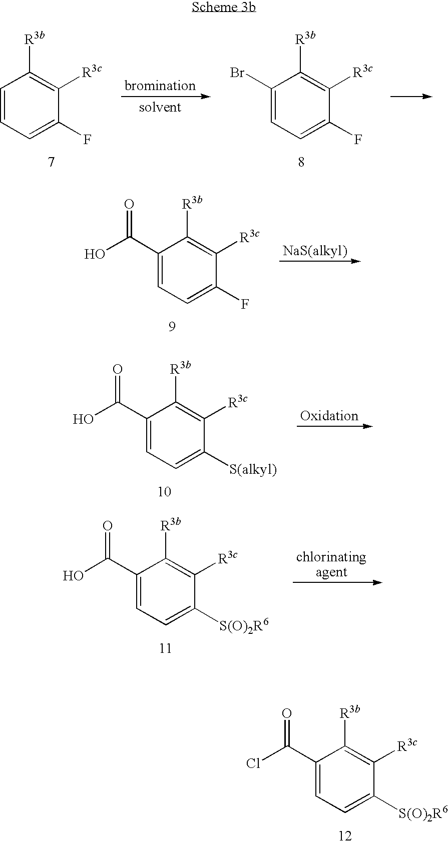

An intermediate of formula 7 where R^{3b} is alkyl, and R^{3c} is halo is commercially available or can be prepared using procedures known to one of ordinary skill in the art. Intermediate 7 is brominated in the presence of iron in a solvent such as chloroform for about 2 hours at room temperature. The product can be distilled to yield an intermediate of formula 8.

Intermediate 8 is then treated with isopropylmagnesium bromide in a solvent such as THF at about 0° C. for about 1 hour. The reaction is then allowed to proceed at room temperature for about 12 hours. C(O)₂ is then introduced over about 2 hours and the reaction is then stirred for another 30 minutes (approximately). The reaction is then quenched with water, solvent removed, and treated with an acid such as HCl to yield a precipitate of the intermediate of formula 9.

Intermediate 9 is then treated with a base such as KOH, in a solvent such as DMSO and allowed to stir for about 30 mins. To make the intermediate where R⁶ is methyl, intermediate 9 is treated with sodium thiomethoxide in the presence of a base such as KOH and allowed to react at a temperature of about 55-50° C. for about 4 hours. Additional base, sodium thiomethoxide, and solvent may need to be added. The reaction is then cooled to about 0° C. and quenched with water, and treated with an acid such as HCl to acidify the mixture to yield an intermediate of formula 10 where alkyl is methyl. To make the intermediate where R⁶ is alkyl other than methyl, the reaction is treated with the appropriate thiol or disulfide in the presence of a catalyst such as CuO, a base such as KOH, and in a solvent such as DMSO to yield an intermediate of formula 10.

An intermediate of formula 10, in a solvent(s) such as acetone and/or water, is then treated with a base such as KOH and/or sodium bicarbonate, and with ozone in portions at about 0° C. over about 2 hours. The mixture was treated with an acid such as HCl and the precipitate collected to yield an intermediate of formula 11.

The intermediate of formula 11 is then treated with a chlorinating agent such as thionyl chloride and allowed to react for about 3 hours at reflux. The mixture was then triturated with DCM to yield an intermediate of formula 12.

The intermediate of formula 12 can then be treated with an intermediate of formula 6, 6a, or 6b using conditions described in Scheme 5, 5a, or 5b to yield a Compound of the Invention of Formula I(s), I(t), or I(u):

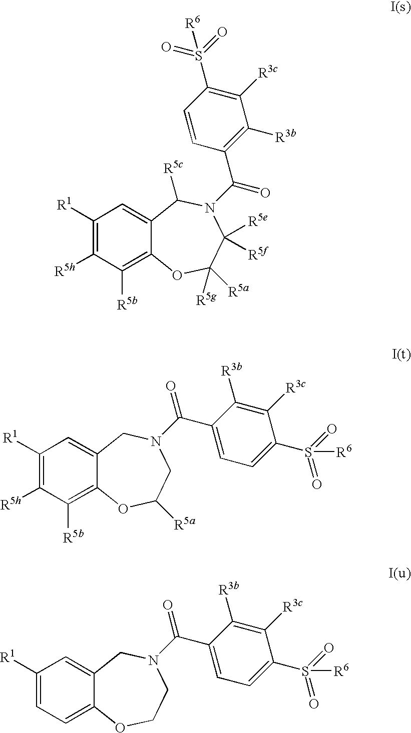

respectively.

A compound of the invention where Z is —C(O)—; R^{5a}, R^{5b}, R^{5c}, R^{5d}, R^{5e}, R^{5f}, R^{5g}, and R^{5h} are hydrogen; R¹ is benzimidazol-6-yl substituted at the 2-position with one R²¹; R²¹ is alkyl; and R² is as defined in the Summary of the Invention for a Compound of Formula I can be prepared according to Scheme 6.

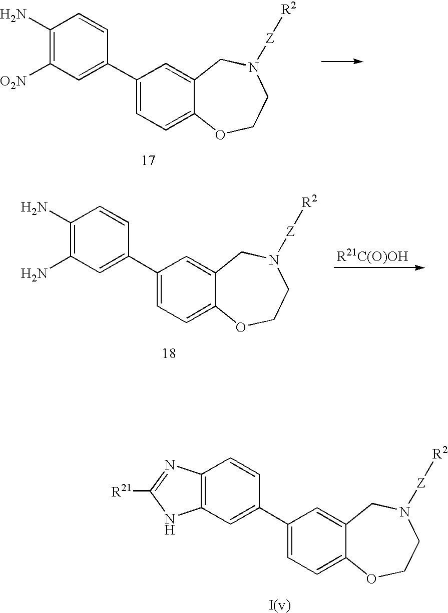

In particular, a Compound of the Invention where Z is —C(O)—; R^{5a}, R^{5b}, R^{5c}, R^{5d}, R^{5e}, R^{5f}, R^{5g}, and R^{5h} are hydrogen; R¹ is benzimidazol-6-yl substituted at the 2-position with one R²¹; R²¹ is alkyl; R² is phenyl substituted with R^{3a}, R^{3b}, R^{3c}, and R^{3d}; and R¹, R^{3a}, R^{3b}, R^{3c}, R^{3d}, and all other groups are as defined in the Summary of the Invention for a Compound of Formula I, can be prepared according to Scheme 6a.

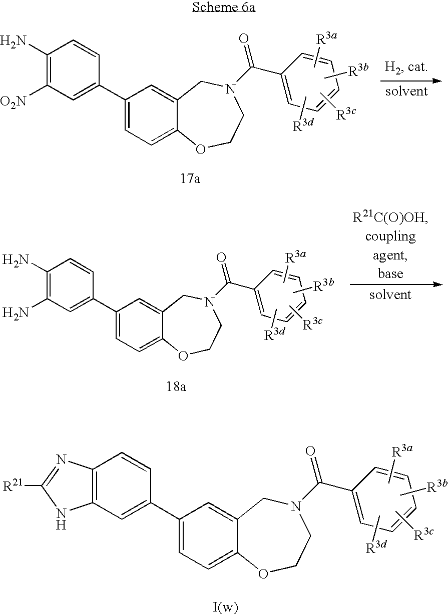

The nitro of the intermediate of formula 17a, prepared as described above in Scheme 4, is reduced in the presence of H₂ and palladium on carbon in a solvent(s) such as methanol and/or acetic acid to yield an intermediate of formula 18a. The intermediate of formula 18a is then treated with an intermediate of formula R²¹C(O)OH, in the presence of a coupling agent such as HATU, in the presence of a base such as DIEA, in a solvent(s) such as DMF and/or acetic acid. The product can be purified by column chromatography to yield a Compound of Formula I(w).

A Compound of Formula I(y) where Z is C(O) and R² is as defined in the Summary of the Invention for a Compound of Formula I can be prepared according to Scheme 7a.

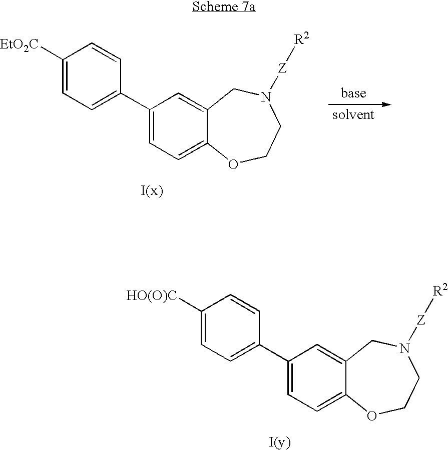

The Compound of Formula I(x), prepared using procedures according to Scheme 5b, is treated with a base such as LiOH, in a solvent(s) such as THF and/or water to yield the hydrolyzed Compound of Formula I(y).

A compound of Formula I(aa) where Z is C(O) and R² and R²⁷ are as defined in the Summary of the Invention for a Compound of Formula I can be prepared using procedures described in Scheme 7a.

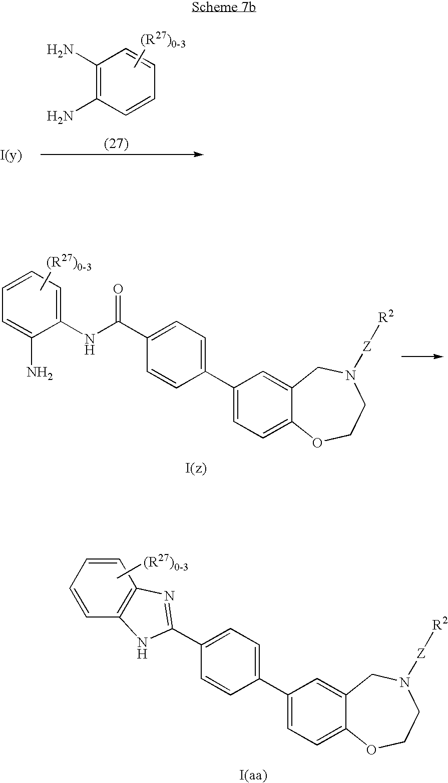

The Compound of Formula I(y) is treated with an intermediate of formula 27, which is commercially available or can be prepared using procedures known to one of ordinary skill in the art, in the presence of a base such as DIEA, a coupling reagent(s) such as HATU and/or HOBt, and in a solvent such as DMA or DMF to yield a Compound of Formula I(z). The Compound of Formula I(z) is then treated with acetic acid at a temperature of about 70° C. for about 1 h to yield a Compound of Formula I(aa).

A Compound of Formula I(cc), I(dd), I(ee), or I(ii) can be prepared according to Scheme 8 where Z is C(O); R² is as defined in the Summary of the Invention for a Compound of Formula I; Ring is the R¹ phenyl or the R¹ heteroaryl; when Ring is phenyl, R^{a} is R²⁰, as defined in the Summary of the Invention for a Compound of Formula I, and when R¹ is heteroaryl, R^{a} is R²¹, as defined in the Summary of the Invention for a Compound of Formula I; and R¹⁸, R^{23a}, R^{15a}, R^{15b}, and R²⁴ are as defined in the Summary of the Invention for a Compound of Formula I.

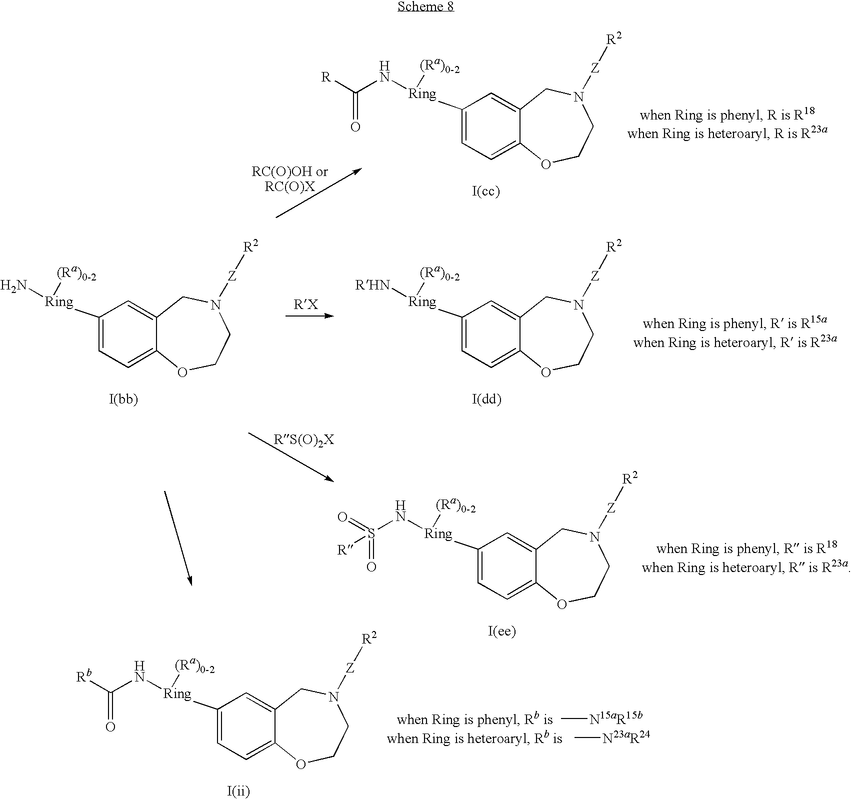

The Compound of Formula I(bb), prepared according to Scheme 5a or 5b, is treated with RC(O)OH or RC(O)X where X is halo using standard amide formation conditions to yield a Compound of Formula I(cc). Alternatively, the Compound of Formula I(bb) is treated with R′X where X is halo using conditions known to one of ordinary skill in the art to yield a Compound of Formula I(dd). Alternatively, the Compound of Formula I(bb) is treated with R″S(O)₂X where X is halo using conditions known to one of ordinary skill in the art to yield a Compound of Formula I(ee). Alternatively, the Compound of Formula I(bb) is treated with R^{b}C(O)OH or R^{b}C(O)X where X is halo using conditions known to one of ordinary skill in the art to yield a Compound of Formula I(ii).

An intermediate of formula 28 can be prepared according to Scheme 9 where PG is a nitrogen-protecting group and Ring is the R¹ phenyl or the R¹ heteroaryl; when Ring is phenyl, R^{a} is R²⁰, as defined in the Summary of the Invention for a Compound of Formula I, and when R¹ is heteroaryl, R^{a} is R²¹, as defined in the Summary of the Invention for a Compound of Formula I; and R^{15a} and R^{23a} are as defined in the Summary of the Invention for a Compound of Formula I.

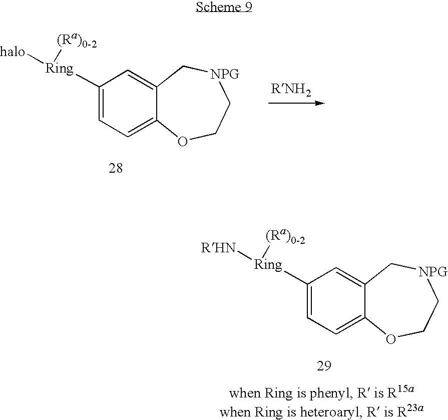

An intermediate of formula 28 is treated with an amine of formula R′NH₂, which is commercially-available or can be prepared using conditions known to one of ordinary skill in the art, in a solvent such n-BuOH at a temperature of about 160° C. for as to yield an intermediate of formula 29. The intermediate of formula 29 can then be deprotected and treated with R²C(O)OH or R²C(O)X where X is halo according to Scheme 5a or 5b to yield a Compound of the Invention of Formula I(dd).

A Compound of Formula I(r) where Z is C(O), R² is as defined in the Summary of the Invention for a Compound of Formula I, and R¹ is 1H-pyrazol-4-yl, 1H-indazol-3-yl, 1H-indazol-5-yl, 1H-indazol-6-yl, 1H-benzimidazol-5-yl, 1H-benzimidazol-6-yl, 2-methyl-1H-benzimidazol-5-yl, 1H-1,2,3-benzotriazol-6-yl, 2-methyl-1H-benzimidazol-6-yl, 1H-imidazo[4,5-b]pyridin-6-yl, 1H-pyrazolo[3,4-b]pyridin-5-yl, 1H-pyrazolo[3,4-b]pyridin-6-yl, or 1H-pyrazolo[3,4-b]pyridin-3-yl, can be treated with an intermediate of formula R²¹X (where X is halo and R²¹ is alkyl, haloalkyl, hydroxyalkyl, alkoxyalkyl, cycloalkyl, cycloalkylalkyl, or alkyl substituted with one —NR²³R^{23a} where R²³ and R^{23a} are independently hydrogen or alkyl). The reaction is carried out in the presence of a base such as K₂CO₃, in a solvent such as DMF to yield a Compound of the Invention where Z is C(O); R² is as defined in the Summary of the Invention for a Compound of Formula I; R¹ is N—(R²¹)-1H-pyrazol-4-yl, N—(R²¹)-1H-indazol-3-yl, N—(R²¹)-1H-indazol-5-yl, N—(R²¹)-1H-indazol-6-yl, N—(R²¹)-1H-benzimidazol-5-yl, N—(R²¹)-1H-benzimidazol-6-yl, N—(R²¹)-2-methyl-1H-benzimidazol-5-yl, N—(R²¹)-1H-1,2,3-benzotriazol-6-yl, N—(R²¹)-2-methyl-1H-benzimidazol-6-yl, N—(R²¹)-1H-imidazo[4,5-b]pyridin-6-yl, N—(R²¹)-1H-pyrazolo[3,4-b]pyridin-5-yl, N—(R²¹)-1H-pyrazolo[3,4-b]pyridin-6-yl, or N—(R²¹)-1H-pyrazolo[3,4-b]pyridin-3-yl; and R²¹ is alkyl, haloalkyl, hydroxyalkyl, alkoxyalkyl, cycloalkyl, cycloalkylalkyl, or alkyl substituted with one —NR²³R^{23a} where R²³ and R^{23a} are independently hydrogen or alkyl.

A Compound of Formula I(jj) where R²¹ is alkyl, haloalkyl, hydroxyalkyl, alkoxyalkyl, cycloalkylalkyl, heteroaryl, heteroarylalkyl, —OR²⁴ (where R²⁴ is alkyl), —C(O)OR²², alkyl substituted with one —NR²³R^{23a}, —NR²³C(O)R^{23a}, alkyl substituted with one —NR²³C(O)R^{24a}, or —NR²³S(O)₂R^{23a}; Z is C(O); and R² is as defined in the Summary of the Invention for a Compound of Formula I can be prepared using procedures according to Scheme 10.

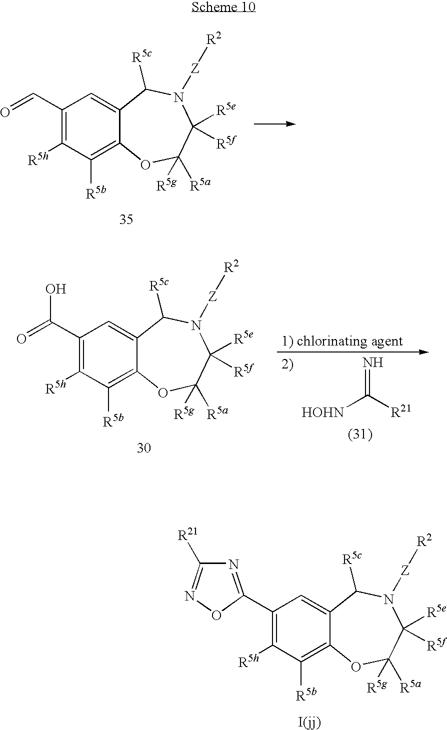

An intermediate of formula 35 is treated with an oxidizing agent such as NaClO₂ in the presence of NaH₂PO₄ in a solvent(s) such as THF and/or t-BuOH to yield the intermediate of formula 30. The intermediate of formula 30 is treated with a chlorinating agent such as (COCl)₂, in the presence of DMF, in a solvent such as benzene and then treated with an intermediate of formula 31, in the presence of a base such as pyridine, in a solvent such as DMA to yield a Compound of Formula I(jj). The Compound of Formula I(jj) where R²¹ is —C(O)OR²² and R²² is alkyl can then be hydrolyzed by treating with a base such as KOH in a solvent(s) such as water and/or ethanol and then treated with an amine of formula NHR²³R^{23a} under standard amide formation conditions to yield a Compound of Formula II(kk):

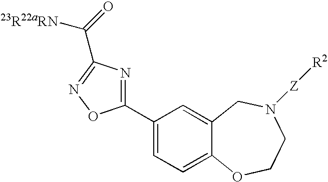

where R²³ and R^{23a} are as defined in the Summary of the Invention for a Compound of Formula I. An intermediate of formula 33 where R^{5a} is hydrogen or alkyl; R^{5h} is hydrogen or halo; R^{5b} is hydrogen, amino, or halo; and R′ is hydrogen or R²¹ and R²¹ is alkyl, haloalkyl, or —NR²³R^{23a}, and R²³ and R^{23a} are as defined in the Summary of the Invention for a Compound of Formula I can be prepared using procedures according to Scheme 11.

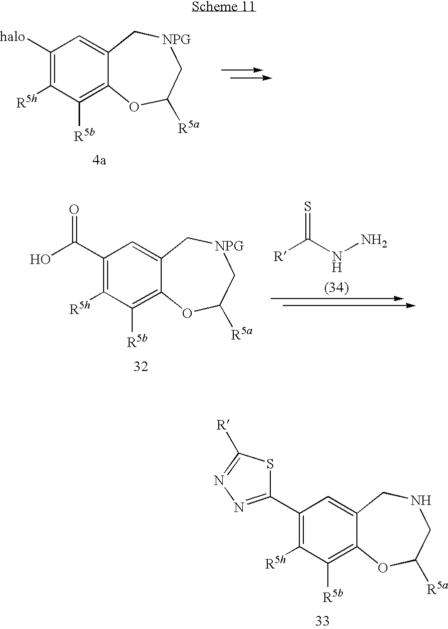

An intermediate of formula 4a can be treated with a base such as n-BuLi in a solvent(s) such as THF and/or DMF, quenched by adding H₂O, and optionally purified, and then followed by treatment with 2,3-dimethyl-2-butene in the presence of NaH₂PO₄, in the presence of an oxidizing agent such as NaClO₂, in a solvent(s) such as THF and/or t-BuOH to yield an intermediate of formula 32. Intermediate 32 can then be treated with an intermediate of formula 34 in the presence of a coupling reagent(s) such as HATU and/or HOBt, in the presence of a base such as DIEA, and in a solvent such as DMF and then treated with an acid such as H₂SO₄ to yield an intermediate of formula 33. Intermediate 33 can then be treated with R²C(O)OH or R²C(O)X where X is halo using standard amide formation conditions to yield a Compound of Formula I(mm):

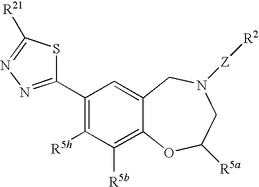

where R^{5a}, R^{5h}, R^{5b}, and R²¹ are as defined above, Z is C(O), and R² is as defined in the Summary of the Invention for a Compound of Formula I.

The Compound of Formula I(mm), when R²¹ is —NH₂, can be treated with R^{24a}C(O)OH or R^{24a}C(O)X where X is halo and R^{24a} is as defined in the Summary of the Invention for a Compound of Formula I using standard coupling conditions to yield a Compound of Formula I(mm) where R²¹ is —NHC(O)R^{24a}. Alternatively, when R²¹ is —NH₂, the Compound of Formula I(mm) can be treated with R^{23a}S(O)₂X where X is halo using conditions known to one of ordinary skill in the art to yield a Compound of Formula I(mm) where R²¹ is —NHS(O)₂R^{23a} and R^{23a} is as defined in the Summary of the Invention for a Compound of Formula I.

A Compound of Formula I where Z, R¹, R², R^{5a}, R^{5b}, R^{5c}, R^{5d}, R^{5e}, R^{5f}, R^{5g} and R^{5h} can be prepared according to the following scheme (where X is halo or hydroxy) using amide formation procedures known to one of ordinary skill in the art.

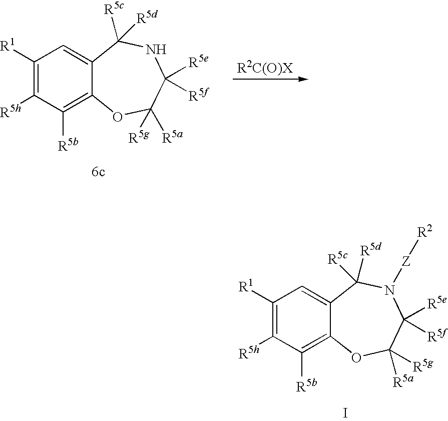

A Compound of Formula I where Z, R¹, R², R^{5a}, R^{5b}, R^{5c}, R^{5d}, R^{5e}, R^{5f}, R^{5g}, and R^{5h} can be prepared according to the following scheme (where R is —B(OH)₂ and Y is halo, or R is halo and Y is —B(OH)₂) using Suzuki coupling procedures known to one of ordinary skill in the art.

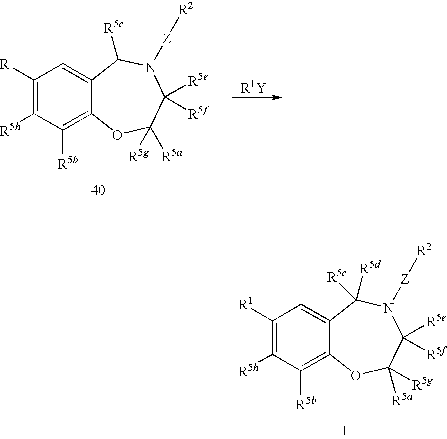

The following R²C(O)OH which can be used in the preparation of a compound of Formula I are commercially available: 4-bromo-benzoic acid; 4-chloro-benzoic acid; 4-iodobenzoic acid; 2,4-dibromo-benzoic acid; 2,4-dichloro-benzoic acid; (2-bromo-4-chlorophenyl)carboxylic acid; 4H-pyrrolo[2,3-d]thiazole-5-carboxylic acid; 4-(2,2,2-trifluoroacetyl)benzoic acid; N-methyl-1H-indole-2-carboxylic acid; 4-fluoro-1H-indole-2-carboxylic acid; 5-fluoro-N-methyl-1H-indole-2-carboxylic acid; 5-chloro-N-methyl-1H-indole-2-carboxylic acid; 5-chloro-N-ethyl-1H-indole-2-carboxylic acid; 5-bromo-N-methyl-1H-indole-2-carboxylic acid; 6-fluoro-1H-indole-2-carboxylic acid; 7-fluoro-1H-indole-2-carboxylic acid; 7-chloro-1H-indole-2-carboxylic acid; 1-ethyl-1H-indole-2-carboxylic acid; 6-methoxy-1H-indole-2-carboxylic acid; [1-methyl-7-(methyloxy)-1H-indol-2-yl]carboxylic acid; [7-(methyloxy)-1H-indol-2-yl]carboxylic acid; 5-carboxy-thiophene-2-carboxylic acid; [1-methyl-5-(methylsulfonyl)-1H-indol-2-yl]carboxylic acid; 2-bromo-4-(trifluoromethyl)benzoic acid; 4-methylsulfonyl-benzoic acid; 4-(ethylsulfonyl)benzoic acid; [4-(n-propylsulfonyl)phenyl]carboxylic acid; (4-methyl-4H-furo[3,2-b]pyrrol-5-yl)carboxylic acid; 4-ethyl-4H-furo[3,2-b]pyrrole-5-carboxylic acid; 4-ethyl-4H-thieno[3,2-b]pyrrole-5-carboxylic acid; 4-methyl-4H-thieno[3,2-b]pyrrole-5-carboxylic acid; 4-ethyl-2-methyl-4H-thieno[3,2-b]pyrrole-5-carboxylic acid; 4-sulfamoylbenzoic acid; 4-(N-methylsulfamoyl)benzoic acid; 4-(N-ethylsulfamoyl)benzoic acid; 4-(N-(2-hydroxyethyl)sulfamoyl)benzoic acid; 4-(N-cyclopropylsulfamoyl)benzoic acid; 4-(N-tert-butylsulfamoyl)benzoic acid; 4-(N-allylsulfamoyl)benzoic acid; 4-(N-(furan-2-ylmethyl)sulfamoyl)benzoic acid; 4-(pyrrolidin-1-ylsulfonyl)benzoic acid; 4-(N-phenylsulfamoyl)benzoic acid; 4-(N-methoxy-N-methylsulfamoyl)benzoic acid; 3-chloro-4-sulfamoylbenzoic acid; 2-methyl-benzoic acid; 2-ethyl-benzoic acid; 4-isopropyl-benzoic acid; 4-tert-butyl-benzoic acid; (4-ethynylphenyl)carboxylic acid; 4-acetyl-benzoic acid; (2-chloro-4-ethyl-4H-thieno[3,2-b]pyrrol-5-yl)carboxylic acid; 4-(1,1,1,3,3,3-hexafluoro-2-hydroxypropan-2-yl)benzoic acid; 4-trifluoromethylsulfonyl-benzoic acid; 6H-thieno[2,3-b]pyrrole-5-carboxylic acid; 4-bromo-2-methylbenzoic acid; 4-chloro-2-methyl-benzoic acid; 4-(methylsulfonamido)benzoic acid; 4-(N-methylmethylsulfonamido)benzoic acid; 4-(N-methylphenylsulfonamido)benzoic acid; (2,4-dimethyl-1,3-thiazol-5-yl)carboxylic acid; 4-(1,2,3-thiadiazol-4-yl)benzoic acid; [4-(1H-imidazol-1-yl)phenyl]carboxylic acid; [4-(1,3-oxazol-5-yl)phenyl]carboxylic acid; 5-carboxy-1H-pyrrole-2-carboxylic acid; benzothien-2-ylcarboxylic acid; (1-ethyl-3-methyl-1H-pyrazol-5-yl)carboxylic acid; 5-(methyloxy)-1H-pyrrolo[2,3-c]pyridin-2-yl]carboxylic acid; (2,3-dimethylphenyl)carboxylic acid; (2,4-dimethylphenyl)carboxylic acid; (2,4,5-trimethylphenyl)carboxylic acid; [1-methyl-4-(pyrrolidin-1-ylsulfonyl)-1H-pyrrol-2-yl]carboxylic acid; [4-(pyrrolidin-1-ylsulfonyl)-1H-pyrrol-2-yl]carboxylic acid; 4-(2-hydroxyethyl)-benzoic acid; 4-cyclopentylsulfonyl-benzoic acid.

4-(2,2,2-trifluoro-1-hydroxyethyl)benzoic acid can be prepared using the procedures in Organic Letters, 2005, 7(11), 2193-2196. (4-chloro-2-ethylphenyl)carboxylic acid and 4-bromo-2-ethylbenzoic acid can be prepared using the procedures in J. of Org. Chem. 2005, 70(4), 1501-1504. 2-methyl-4-(methylsulfonyl)benzoic acid can be prepared using procedures in U.S. Pat. No. 4,925,970. 2-Bromo-4-(methylsulfonyl)-benzoic acid can be prepared using procedures described in WO9006301. 4-(tert-Butylaminocarbonyl)-benzoic acid can be prepared using procedures described in Organic Letters 2008, 10(8), 1589-1592.

### SYNTHETIC EXAMPLES

### Reference Example 1

### 3-Fluoro-2-methyl-4-(methylsulfonyl)benzoyl chloride

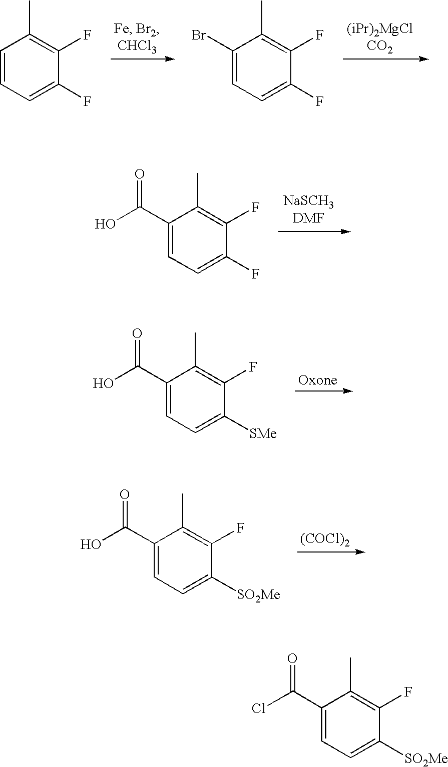

1-Bromo-3,4-difluoro-2-methylbenzene. To a stirred mixture of 2,3-difluorotoluene (1.9 g, 14.8 mmol) and iron (82.7 mg, 1.48 mmol) in chloroform (10 mL) at rt was added bromine (76 μL, 14.8 mmol) over 2 h. The resulting mixture was stirred at rt overnight. Excess water (10 mL) was added and the reaction mixture was diluted with ether (20 mL). The separated organic layer was washed with aqueous sodium thiosulfate, brine, dried over sodium sulfate and concentrated on a rotary evaporator. The residue was distilled to give the desired product (2.49 g, 81%) as a colorless oil.

3,4-Difluoro-2-methylbenzoic acid. To a stirred solution of 1-bromo-3,4-difluoro-2-methylbenzene (940 mg, 4.54 mmol) in tetrahydrofuran (5 mL) was added isopropylmagnesium bromide (3.0 mL, 6.0 mmol) over 1 h at 0° C. The resulting mixture was stirred at rt for 24 h. Carbon dioxide (CO₂), generated from dry ice, was introduced to the reaction mixture over 2 h and the resulting mixture was stirred for an additional 30 min. The reaction mixture was quenched with addition of an excess amount of water (5 mL) and the tetrahydrofuran was removed on a rotary evaporator. The resulting aqueous layer was diluted with water (5 mL) and acidified with concentrated hydrochloric acid to pH 1-2. The white precipitate was filtered and washed with water and cold hexanes and dried under high vacuum to give the desired product (630 mg, 81%) as a white powder. MS (EI) for C₈H₆F₂O₂: 171 (MH⁻).

3-Fluoro-2-methyl-4-(thiomethyl)benzoic acid. To a stirred solution of acid 3,4-difluoro-2-methylbenzoic acid (700 mg, 4.1 mmol) in dimethylsulfoxide (5 mL) was added powdered potassium hydroxide (274 mg, 4.9 mmol) and the mixture was stirred at rt for 30 min. Sodium thiomethoxide (342 mg, 4.9 mmol) was added to the mixture and the resulting mixture was stirred at 55-60° C. for 4 h. Additional powdered potassium hydroxide (70 mg, 1.2 mmol), sodium thiomethoxide (60 mg, 0.8 mmol), and dimethylsulfoxide (2 mL) were added to the reaction mixture. After stirring for 4 h, the mixture was cooled to 0° C. and quenched with excess water (10 mL). The resulting suspension was acidified at 0° C. with concentrated hydrochloric acid to pH 1-2. The white precipitate was collected by suction filtration, washed with water and dried under vacuum overnight to give the desired product (870 mg, 100%). The intermediate sulfide was used in the next step without further purification. MS (EI) for C₉H₉FO₂S: 199.1 (MH⁻).

3-Fluoro-2-methyl-4-(methylsulfonyl)benzoic acid. To a stirred suspension of 3-fluoro-2-methyl-4-(thiomethyl)benzoic acid in an acetone/water (1 mL/10 mL) mixture was added sodium hydroxide (330 mg, 8.25 mmol) and sodium bicarbonate (680 mg, 8.1 mmol). Oxone (˜4 g) was added portionwise to the reaction mixture at 0° C. over 2 h. The reaction was monitored by LC/MS. Concentrated hydrochloric acid was added to adjust the pH 2-3 and the white precipitate was collected by suction filtration, washed with water, and dried under vacuum. Dried precipitate was suspended in water (10 mL), stirred vigorously at rt for 1 h, filtered, washed with water, and hexanes and dried under vacuum to give the desired product (886 mg, 94%) as a white powder. MS (EI) for C₉H₉FO₄S: 231 (MH⁻).

3-Fluoro-2-methyl-4-(methylsulfonyl)benzoyl chloride. A mixture of 3-fluoro-2-methyl-4-(methylsulfonyl)benzoic acid (860 mg, 3.7 mmol) in thionyl chloride (10 mL) was heated to reflux for 3 h. (the reaction mixture became homogenous). The reaction mixture was concentrated on a rotary evaporator to give the crude acid chloride. This acid chloride was triturated with dichloromethane (2 mL) and concentrated under reduced pressure. The trituration process was repeated 3 times until the product (900 mg, 98%) was obtained as a white powder.

### Reference Example 2

### Ethyl 4-(2,3,4,5-tetrahydro-1,4-benzoxazepin-7-yl)benzoate hydrochloride salt

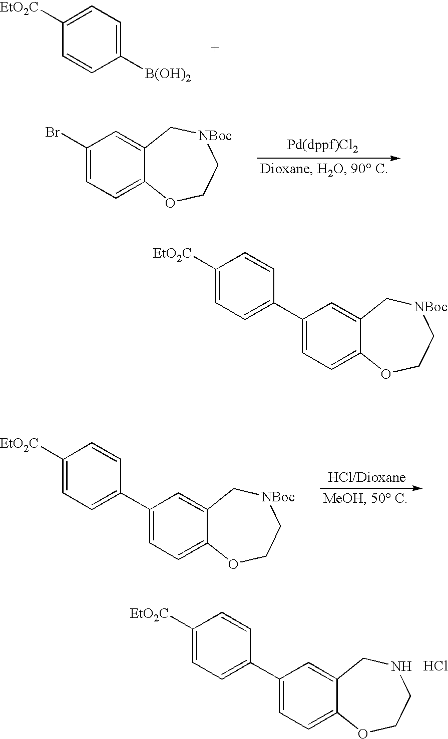

4-(ethoxycarbonyl)phenylboronic acid (22.16 g, 114 mmol), tert-butyl 7-bromo-2,3-dihydrobenzo[f][1,4]oxazepin-4(5H)-carboxylate (34.08 g, 104 mmol), prepared as described in Reference Example 4, Pd(dppf)Cl₂ and TEA (21 g, 208 mmol) were combined in a mixture of dioxane (200 mL) and water (20 mL). The reaction mixture was heated to 90° C. for 2 h, then cooled and the solvent removed. Purification of the residue by silica chromatography gave the desired product ester (31.3 g, 69% yield).

To the solution of tert-butyl 7-(4-(ethoxycarbonyl)phenyl)-2,3-dihydrobenzo[f][1,4]oxazepine-4(5H)-carboxylate (10.3 g, 25.93 mmol) in MeOH (120 mL) was added a solution of 4 N HCl in dioxane (50 mL). The reaction mixture was heated to 50° C. for 3 h (monitored by LC/MS). The reaction mixture was allowed to cool to rt. Ethyl 4-(2,3,4,5-tetrahydro-1,4-benzoxazepin-7-yl)benzoate as the hydrochloride salt (8.8 g, 99% yield) was collected by suction filtration.

### Reference Example 3

### 2-Ethyl-3-fluoro-4-(methylsulfonyl)benzoic acid

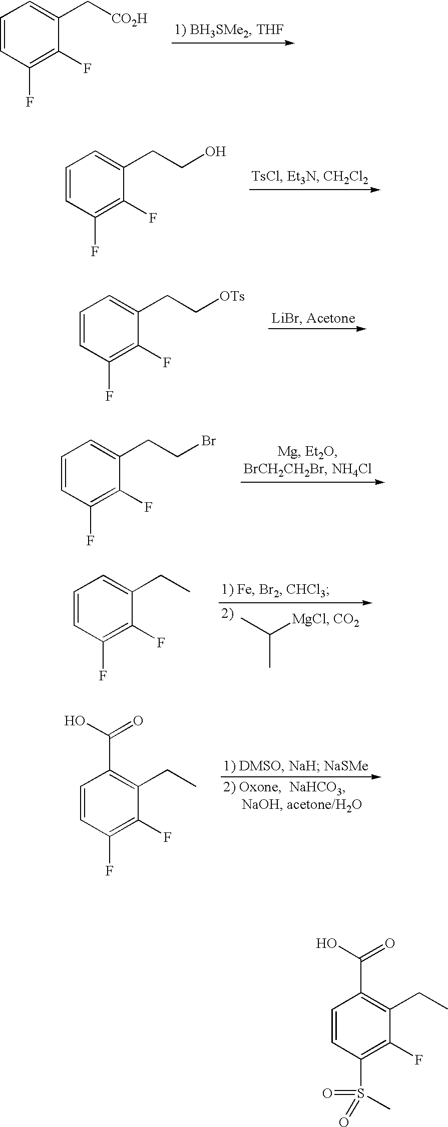

To a stirred solution of commercially-available (2,3-difluorophenyl)acetic acid (17.2 g, 100 mmol) in THF (150 mL) was added BH₃-Me₂S (75 mL, 2 M in THF) using dropping funnel over 1 h at rt. The reaction mixture started to reflux spontaneously. After stirring for additional 3 h, excess MeOH (20 mL) was added carefully to the reaction mixture at 0° C. (ice bath). After vigorous stirring for 1 h, most of solvents were removed under reduced pressure. The residue was diluted with CH₂Cl₂ (100 mL), washed with aqueous NaHCO₃ (100 mL), dried with Na₂SO₄, and concentrated under reduced pressure to afford 2-(2,3-difluorophenyl)ethanol (15.6 g, 99%) which was used in the next step without further purification. GC/MS=158 (M+)

To a stirred solution of 2-(2,3-difluorophenyl)ethanol (15.6 g, 98.6 mmol) and Et₃N (20.6 mL, 148 mmol) in CH₂Cl₂ (100 mL) was added TsCl (20.7 g, 109 mmol). The reaction mixture was stirred for 5 h at rt. The reaction mixture was diluted with CH₂Cl₂ (200 mL), washed with H₂O (100 mL), brine (100 mL), dried with Na₂SO₄, concentrated under reduced pressure to give 2-(2,3-difluorophenyl)ethyl 4-methylbenzenesulfonate (29.6 g, 96%).

To a stirred solution of 2-(2,3-difluorophenyl)ethyl 4-methylbenzenesulfonate (29.6 g, 94.8 mmol) in acetone (150 mL) was added LiBr (42.8 g, 492 mmol) and the reaction mixture was stirred at reflux for 3 h. The reaction mixture was cooled to rt and concentrated under reduced pressure. The residue was partitioned with H₂O (200 mL)/hexanes (200 mL). The separated aqueous layer was extracted with hexanes (100 mL). The combined organic layer was washed with brine (100 mL), dried with Na₂SO₄, and concentrated under reduced pressure. The residue was purified by flash chromatography to give 1-(2-bromoethyl)-2,3-difluorobenzene (19.37 g, 89%). GC/MS=142 (M-Br).

To a stirred mixture of Mg (4.26 g, 175 mmol) in Et₂O (50 mL) was added 1,2-dibromoethane (0.755 mL, 8.76 mmol) and the mixture was allowed to stir for 10 min. To this reaction mixture was added a solution of 1-(2-bromoethyl)-2,3-difluorobenzene (19.37 g, 87.6 mmol) and 1,2-dibromoethane (0.755 mL, 8.76 mmol) in Et₂O (50 mL) over 30 min at refluxing temperature. Additional stirring was continued for 1 h. The reaction mixture was gradually cooled to rt. then to 0° C. using ice bath, and carefully quenched by adding aqueous NH₄Cl (50 mL) over 1 h. The resulting mixture was diluted with a mixture of H₂O (100 mL) and Et₂O (100 mL). The separated organic layer was washed with brine (100 mL), dried with Na₂SO₄, and filtered. Vacuum distillation of filtrate gave 1-ethyl-2,3-difluorobenzene (9.5 g, 76%). GC/MS=142 (M+).

To a stirred mixture of 1-ethyl-2,3-difluorobenzene (9.5 g, 66.8 mmol) and Fe (373 mg, 6.68 mmol) in CHCl₃ (10 mL) was added Br₂ (3.43 mL, 66.9 mmol) dropwise using a dropping funnel over 1 h and the reaction mixture was stirred at rt overnight. The reaction mixture was diluted with a mixture of H₂O (50 mL)/Et₂O (100 mL) and the separated aqueous layer was extracted with Et₂O (100 mL). The combined organic extracts were dried with Na₂SO₄, concentrated under reduced pressure, and the residue purified by flash chromatography to give 1-bromo-2-ethyl-3,4-difluorobenzene (10.5 g, 71%).

To a stirred solution of 1-bromo-2-ethyl-3,4-difluorobenzene (10.5 g, 47.5 mmol) in THF (75 mL) was added isopropyl magnesium chloride (35.6 mL, 71.2 mmol) dropwise over 30 min at 0° C. The resulting mixture was stirred at rt overnight. Carbon dioxide (generated from dry ice) was introduced to the reaction mixture over 2 h and the resulting mixture was stirred for additional 1 h. The reaction mixture was quenched by adding H₂O (100 mL) and most of THF was removed under reduced pressure. The residue was acidified to pH 1-2 with 2 N HCl and extracted with CH₂Cl₂ (4×100 mL). The combined organic layer was dried with Na₂SO₄, and concentrated under reduced pressure to give acid. This acid was triturated with hexanes and collected 2-ethyl-3,4-difluorobenzoic acid, as a white precipitate (8.31 g, 94%). LC/MS=185 (MH⁻).

To a stirred solution of acid 2-ethyl-3,4-difluorobenzoic acid (2.0 g, 10.7 mmol) in DMSO (15 mL) was added NaSMe (1.87 g, 26.7 mmol) and the resulting mixture was stirred for 5 h at 60° C. The reaction mixture was cooled to rt, quenched with H₂O (50 mL), acidified with conc. HCl to pH 1-2, extracted with CH₂Cl₂ (3×50 mL), and the organic extracts were dried with Na₂SO₄, concentrated under reduced pressure. The crude sulfide was used in the next step without further purification. LC/MS=213 (MH⁻).

To a stirred solution of crude sulfide in acetone (20 mL)/H₂O (20 mL) was added NaOH (800 mg, 20 mmol), NaHCO₃ (4.5 g, 53.6 mmol), and oxone (10 g). After vigorous stirring for 1 h at rt, the reaction mixture was concentrated at reduced pressure. The residue was acidified with conc. HCl to pH 1-2, the precipitate collected by suction filtration, and air dried. The precipitate was triturated with H₂O (50 mL), filtered, washed with H₂O (2×50 mL), hexanes, and dried under vacuum to give compound 2-ethyl-3-fluoro-4-(methylsulfonyl)benzoic acid (2.3 g, 87% in 2 steps). LC/MS=245 (MH⁻).

Using the same or analogous synthetic techniques and substituting with appropriate reagents, 2-ethyl-3-fluoro-4-(ethylsulfonyl)benzoic acid was prepared.

### Reference Example 4

### tert-Butyl-7-bromo-2,3-dihydrobenzo[f][1,4]oxazepine-4(5H)-carboxylate

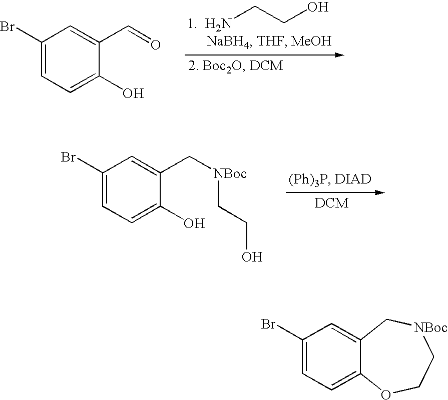

tert-Butyl-5-bromo-2-hydroxybenzyl(2-hydroxyethyl)carbamate. Commercially-available 5-bromo-2-hydroxybenzaldehyde (4.0 g, 10 mmol) and 2-aminoethanol were combined in THF/MeOH (100 mL, 10:1) and sodium borohydride (0.76 g, 2.0 mmol) was added with stirring. The resulting reaction mixture was stirred at 40° C. for 4 h, concentrated on a rotary evaporator then diluted with EtOAc (50 mL) and saturated NaHCO₃ (30 mL). To this suspension was added di-tert-butyl dicarbonate (2.83 g, 13 mmol). The mixture was stirred at rt overnight. The organic layer was washed with water, dried over anhydrous magnesium sulfate, filtered, and concentrated on a rotary evaporator. Hexane was subsequently added to the crude reaction product which resulted in the formation of a white solid. This slurry was filtered to obtain the desired product (6.8 g, 98%) as a white solid. MS (EI) for C₁₄H₂₀BrNO₄, found 346 (MH+).

tert-Butyl-7-bromo-2,3-dihydrobenzo[f][1,4]oxazepine-4(5H)-carboxylate. tert-Butyl-5-bromo-2-hydroxybenzyl(2-hydroxyethyl)carbamate (3.46 g, 10 mmol) and triphenylphosphine (3.96 g, 15 mmol) were combined in DCM (100 mL) and diisopropyl azodicarboxylate (3.03 g, 15 mmol) was added. The resulting reaction mixture was stirred at rt for 12 h. The reaction mixture was washed with water, dried, filtered, and concentrated on a rotary evaporator. The resulting crude product was purified via silica gel chromatography eluting with 8:2 hexane/ethyl acetate to give the desired product (1.74 g, 53%) as a white solid. MS (EI) for C₁₄H₁₈BrNO₃, found 328 (MH+).

### Reference Example 5

### 4-(tert-Butoxycarbonyl)-2,3,4,5-tetrahydrobenzo[f][1,4]oxazepin-7-ylboronic acid

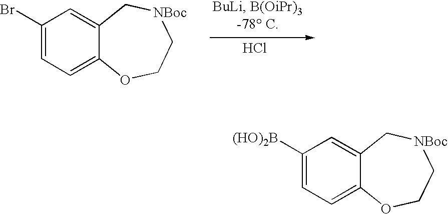

To a stirred solution of tert-butyl-7-bromo-2,3-dihydrobenzo[f][1,4]oxazepine-4(5H)-carboxylate (10 g, 30.5 mmol), prepared as described in Reference Example 4, and triisopropylborate (9.1 mL, 40 mmol) in dry tetrahydrofuran (100 mL) was added dropwise n-butyllithium in tetrahydrofuran (1.6 M, 25 mL, 40 mmol) while maintaining the temperature below −60° C. Upon completion of addition, the reaction mixture was stirred for 30 min, then quenched with 1 N aqueous hydrochloric acid (35 mL) and allowed to warm to rt. The reaction mixture was extracted with ethyl acetate, dried over anhydrous magnesium sulfate, filtered and concentrated on a rotary evaporator. Hexane was subsequently added to the crude reaction product which resulted in the formation of a white solid. This slurry was stirred for 1 h and filtered to obtain 4-(tert-butoxycarbonyl)-2,3,4,5-tetrahydrobenzo[f][1,4]oxazepin-7-ylboronic acid (8.6 g, 95%) as a white solid. MS (EI) for C₁₄H₂₀BNO₅: 194 (M-Boc).

### Reference Example 6

### 7-(2-Methyl-1H-benzo[d]imidazol-6-yl)-2,3,4,5-tetrahydrobenzo[f][1,4]oxazepine dihydrochloride

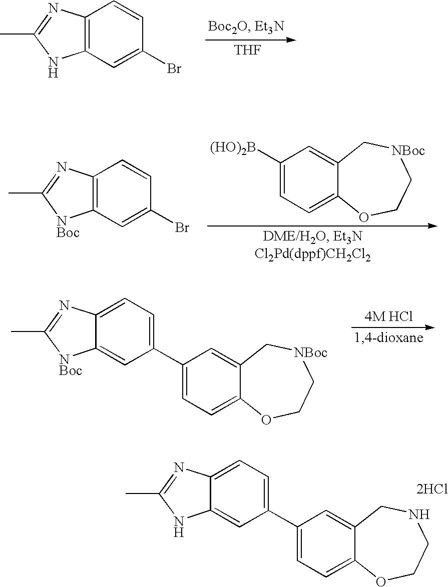

To a solution of commercially-available 5-bromo-2-methylbenzimidazole (5.0 g, 23.6 mmol) and di-tert-butyl dicarbonate (5.68 g, 26.1 mmol) in THF (100 mL) was added Et₃N (9.7 mL, 71.1 mmol). The reaction mixture was stirred at rt overnight, concentrated under reduced pressure, and purification by flash chromatography gave tert-butyl 5-bromo-2-methyl-1H-benzo[d]imidazole-1-carboxylate (6.57 g, 89%).

To a stirred mixture of the tert-butyl 5-bromo-2-methyl-1H-benzo[c/]imidazole-1-carboxylate (6.57 g, 22.4 mmol), 4-(tert-butoxycarbonyl)-2,3,4,5-tetrahydrobenzo[f][1,4]oxazepin-7-ylboronic acid (6.97 g, 22.4 mmol), prepared using procedures described in Reference Example 5, and Et₃N (7.81 mL, 56.0 mmol) in DME (100 mL)/H₂O (10 mL) was added palladium (II) dichloromethane adduct (3.7 g, 4.50 mmol). The resulting mixture was stirred at reflux for 4 h, cooled to rt, concentrated under reduced pressure, and purified by flash chromatography to give tert-butyl 7-(1-(tert-butoxycarbonyl)-2-methyl-1H-benzo[d]imidazol-6-yl)-2,3-dihydrobenzo[f][1,4]oxazepine-4(5H)-carboxylate (5.8 g, 54%).

To a stirred mixture of the tert-butyl 7-(1-(tert-butoxycarbonyl)-2-methyl-1H-benzo[d]imidazol-6-yl)-2,3-dihydrobenzo[f][1,4]oxazepine-4(5H)-carboxylate (5.8 g, 12.1 mmol) in 1,4-dioxane (30 mL) was added 4 N HCl in dioxane (20 mL) and the mixture was stirred at rt overnight. Concentration of the mixture at reduced pressure gave 7-(2-methyl-1H-benzo[d]imidazol-6-yl)-2,3,4,5-tetrahydrobenzo[f][1,4]oxazepine dihydrochloride (3.7 g, 86%).

### Reference Example 7

### 2-bromo-4-fluoro-5-methoxybenzoic acid

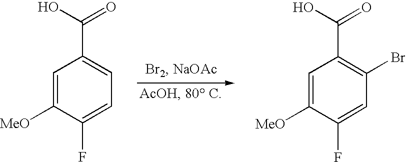

To a stirred suspension of 4-fluoro-3-methoxybenzoic acid (510 mg, 3.0 mmol) in AcOH (10 mL) was added bromine (154 μL, 3.0 mmol) and sodium acetate (492 mg, 6.0 mmol) and the resulting mixture was stirred at 80° C. overnight. Additional amount of bromine (77 μL, 1.5 mmol) and sodium acetate (246 mg, 3.0 mmol) were added to the reaction mixture. After stirring for an additional 24 h at 80° C., the reaction mixture was concentrated under reduced pressure and the residue was diluted with H₂O (50 mL), acidified with 1 N HCl to pH 1-2, and extracted with CH₂Cl₂ (3×50 mL). The combined extracts were dried with Na₂SO₄, concentrated under reduced pressure, and the residue was triturated with hexanes and 2-bromo-4-fluoro-5-methoxybenzoic acid (497 mg, 67%) was obtained by filtration. MS (EI) for C₈H₆BrFO₃, found 247, 249 (MH−).

### Reference Example 8

### 4-(2-chlorophenylsulfonyl)-2-methylbenzoic acid

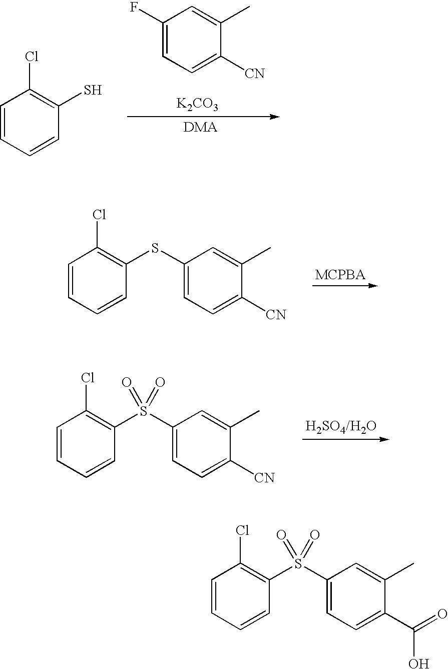

4-(2-chlorophenylthio)-2-methylbenzonitrile. To a mixture of commercially-available 2-chlorobenzenethiol (0.28 g, 2.0 mmol), commercially-available 4-fluoro-2-methylbenzonitrile (0.81 g, 6.0 mmol), and potassium carbonate were added to DMA (2 mL), the mixture was heated at 100° C. for 2 h. The reaction mixture was diluted with ethyl acetate (20 mL), and washed with water, followed by brine. Organic layer was separated, dried over anhydrous MgSO₄ and the solvent was removed at reduced pressure. Purification of the crude product by flash chromatography furnished 4-(2-chlorophenylthio)-2-methylbenzonitrile (0.52 g, 95%).

4-(2-chlorophenylsulfonyl)-2-methylbenzonitrile. To a solution of 4-(2-chlorophenylthio)-2-methylbenzonitrile (0.52 g, 2.0 mmol) in CH₂Cl₂ (50 mL) was added 2-3 equiv. of m-chloroperbenzoic acid. The resulting mixture was stirred for 1 h, washed with concentrated sodium bisulfite, 1 M NaOH, water, and brine. The organic fractions were dried over MgSO₄ and the solvent was removed at reduced pressure. Purification of the crude product by flash chromatography gave the desired product (0.45 g, 77%).

4-(2-chlorophenylsulfonyl)-2-methylbenzoic acid. A mixture of 4-(2-chlorophenylsulfonyl)-2-methylbenzonitrile (0.44 g, 1.50 mmol), acetic acid (5 mL) and 50% sulfuric acid (5 mL) was stirred overnight, then poured into cold water (30 mL), stirred for 15 min. Solid was collected by filtration, washed with water and dried to give desired product (0.30 g, 67%).

### Reference Example 9

### 4-(2-Ethyl-3-fluoro-4-(methylsulfonyl)benzoyl)-2,3,4,5-tetrahydrobenzo[f][1,4]oxazepine-7-carboxylic acid

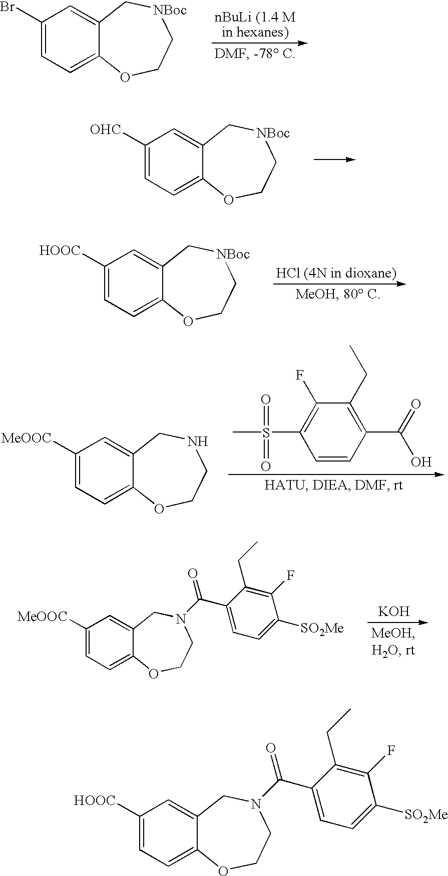

To Boc-bromo-benzoxazepine (8.0 g, 24.4 mmol), prepared as described in Reference Example 4, in THF (50 mL) at −78° C. was added n-BuLi (1.4 M in hexanes, 17.4 mL, 24.4 mmol) dropwise. The mixture was stirred for 15 min then DMF (3.77 mL, 48.8 mmol) was added dropwise. The reaction mixture was allowed to warm to 0° C. over 1 hr then quenched with NH₄Cl (sat.), diluted with EtOAc, washed with brine, dried with Na₂SO₄, filtered, and concentrated to provide tert-butyl 7-formyl-2,3-dihydrobenzo[f][1,4]oxazepine-4(5H)-carboxylate which was carried forward without further purification.

To tert-butyl 7-formyl-2,3-dihydrobenzo[f][1,4]oxazepine-4(5H)-carboxylate (6.5 g, 23.5 mmol) was added THF (20 mL), t-BuOH (10 mL), 2-methyl-2-butene (10.0 mL, 94.4 mmol), NaH₂PO₄ (5.64 g, 47.0 mmol), and H₂O (20 mL). The stirring reaction mixture was placed in an H₂O bath and NaClO₂ (5.08, 56.4 mmol) was added portionwise over 15 min. After stirring at rt for 30 min, the reaction mixture was diluted with NH₄Cl (sat.), extracted with EtOAc, dried with Na₂SO₄, filtered, and concentrated. The crude product was precipitated from hexanes to provide 3.5 g of 4-(tert-butoxycarbonyl)-2,3,4,5-tetrahydrobenzo[f][1,4]oxazepine-7-carboxylic acid (49% over 2 steps).

To 4-(tert-butoxycarbonyl)-2,3,4,5-tetrahydrobenzo[f][1,4]oxazepine-7-carboxylic acid (3.5 g, 11.9 mmol) in MeOH (15 mL) was added HCl (15 mL, 4N in dioxane, 60 mmol)) and the reaction mixture was heated to 80° C. for 2 hrs then quenched with NaHCO₃ (sat.) slowly until basic. The crude product was extracted with EtOAc, washed with brine, dried with Na₂SO₄, filtered, and concentrated to provide methyl 2,3,4,5-tetrahydrobenzo[f][1,4]oxazepine-7-carboxylate as a colorless oil which was carried forward without further purification.

To methyl 2,3,4,5-tetrahydrobenzo[f][1,4]oxazepine-7-carboxylate (1.68 g, 8.08 mmol) was added DMF (10 mL), DIEA (4.0 mL, 23 mmol), 2-ethyl-3-fluoro-4-(methylsulfonyl)benzoic acid (1.99 g, 8.08 mmol), and HATU (3.68 g, 9.68 mmol). The reaction mixture was stirred at rt for 30 min then quenched with NaHCO₃ (sat.), extracted with EtOAc, washed with brine, dried with Na₂SO₄, filtered, and concentrated to provide methyl 4-(2-ethyl-3-fluoro-4-(methylsulfonyl)benzoyl)-2,3,4,5-tetrahydrobenzo[f][1,4]oxazepine-7-carboxylate which was carried forward without further purification.

To methyl 4-(2-ethyl-3-fluoro-4-(methylsulfonyl)benzoyl)-2,3,4,5-tetrahydrobenzo[f][1,4]oxazepine-7-carboxylate (3.51 g, 8.08 mmol) in MeOH (30 mL) was added KOH (20 ml, of a 4 N solution, 80 mmol). After the reaction mixture was stirred at rt for 30 min it was washed with EtOAc and acidified with 1N HCl. The aqueous layer was extracted with EtOAc, dried with Na₂SO₄, filtered, and concentrated to provide 4-(2-ethyl-3-fluoro-4-(methylsulfonyl)benzoyl)-2,3,4,5-tetrahydrobenzo[f][1,4]oxazepine-7-carboxylic acid as a colorless oil. MS (EI) for C₂₀H₂₀FNO₆S, found 422 (MH+).

### Reference Example 10

### 2-Ethyl-3-fluoro-4-(2-hydroxyethylsulfonyl)benzoic acid

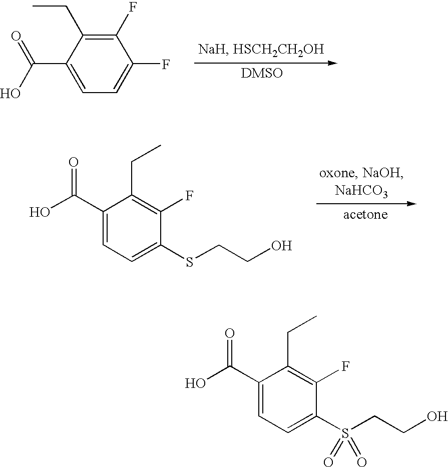

To a solution of 2-ethyl-3,4-difluorobenzoic acid (1.95 g, 10.5 mmol) in DMSO (50 mL) was added sodium hydride (1.00 g, 41.8 mmol) and 2-mercaptoethanol. The resulting mixture was stirred at rt for 30 min and heated at 60° C. for 8 h. The resulting mixture was acidified with aqueous 2 N HCl, extracted with ethyl acetate and concentrated to give 2-ethyl-3-fluoro-4-(2-hydroxyethylthio)benzoic acid which was used in the next step without further purification.

To a solution of 2-ethyl-3-fluoro-4-(2-hydroxyethylthio)benzoic acid (712 mg, 2.91 mmol), aqueous 2 N NaOH (1.5 mL, 2.91 mmol), acetone (5 mL), and NaHCO₃ (734 mg, 8.74 mmol) was added oxone (2.7 g) and the mixture was stirred for 4 h at rt. The mixture was acidified with 2 N HCl, extracted with ethyl acetate and concentrated to give 2-ethyl-3-fluoro-4-(2-hydroxyethylsulfonyl)benzoic acid.

### Reference Example 11

### 4-(N-tert-Butylsulfamoyl) 2-chlorobenzoic acid

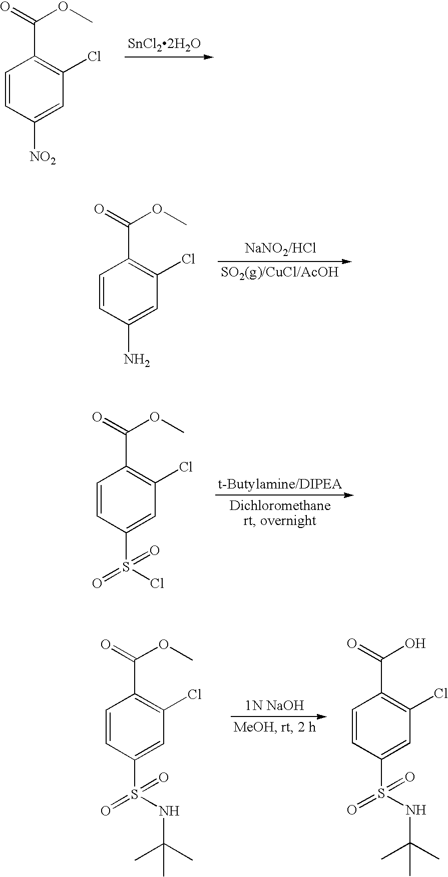

Methyl 4-amino-2-chlorobenzoate. A solution of 2-methyl 2-chloro-4-nitrobenzote (4.50 g, 20.8 mmol) and tin(II) chloride didhydrate (83.5 mmol) in EtOAc (100 mL) was heated to 70° C. and stirred for 1 h. After the reaction was cooled to room temperature, it was diluted with EtOAc (50 mL), washed with H₂O (25 mL) and sat. NaCl solution (25 mL), dried with Na₂SO₄, filtered, concentrated under reduced pressure to afford 4-amino-2-chlorobenzoate (1.80 g, 46.5% yield) as a white solid. MS (EI) for C₈H₈ClNO₂, found 186.2 (MH⁺).

Methyl 2-chloro-4-(chlorosulfonyl)benzoate. An aqueous solution of sodium nitrate (279 mg, 4.05 mmol) in H₂O (4 mL) was added to a suspension of methyl 4-amino-2-chlorobenzoate (500 mg, 2.70 mmol) in conc. HCl (4 mL) at 0° C. and the reaction was stirred for 45 min. The resulting solution was transferred to a reaction mixture of saturated sulfur dioxide acetic acid solution in the presence of CuCl (80 mg, 0.81 mmol) for 15 min. keeping reaction temperature below 5° C. and stirred for 1.5 h. The reaction was diluted with H₂O (25 mL) and extracted with EtOAc (50 mL). The organic layer was washed with H₂O (10 mL), sat. NaHCO₃ (10 mL) and H₂O (10 mL), dried with Na₂SO₄, filtered, concentrated under reduced pressure to afford methyl 2-chloro-4-(chlorosulfonyl)benzoate (570 mg, 78.6% yield). MS (EI) for C₈H₆Cl₂O₄S, found 235 (MH⁺, as SO₂H).

Methyl 4-(N-tert-butylsulfamoyl)-2-chlorobenzoate. To a stirred solution of tert-butylamine (139 μL, 1.30 mmol) and DIEA (303 μl, 1.89 mmol) in dichloromethane (5 mL) was added a dichloromethane (5 mL) solution of methyl 2-chloro-4-(chlorosulfonyl)benzoate 3 (200 mg, 0.74 mmol). The reaction was stirred for 1 h, diluted with 0.5 N HCl (10 mL) and extracted with dichloromethane (20 mL). The organic layer was washed with sat. NaCl solution (10 mL), dried with Na₂SO₄, filtered, concentrated under reduced pressure to afford to afford methyl 4-(N-tert-butylsulfamoyl)-2-chlorobenzoate (235 mg, 94.7% yield). MS (EI) for C₁₂H₁₆ClNO₄S, found 306.2 (MH⁺).

4-(N-tert-Butylsulfamoyl)-2-chlorobenzoic acid. The reaction mixture of methyl 4-(N-tert-butylsulfamoyl)-2-chlorobenzoate 4 (235 mg, 0.77 mmol), 1 M NaOH (5 mL) and MeOH (5 mL) was stirred for 2 h at room temperature. The reaction was evaporated under reduced pressure, diluted with H₂O and adjusted pH of reaction to pH 3.0 by the addition of 1 N HCl. It was extracted with EtOAc (20 mL) and the organic layer was dried with Na₂SO₄, filtered, concentrated under reduced pressure to afford 4-(N-tert-butylsulfamoyl) 2-chlorobenzoic acid (123 mg, 54.9% yield). MS (EI) for C₁₁H₁₄ClNO₄S, found 292.4 (MH⁺).

Using the same or analogous synthetic techniques and substituting with appropriate reagents, the following intermediates were prepared: 4-(N-ethylsulfamoyl)-2-methylbenzoic acid; 4-(N-cyclopentylsulfamoyl)-2-methylbenzoic acid; 4-(N-isopropylpentylsulfamoyl)-2-bromobenzoic acid; 4-(N-ethylsulfamoyl)-2-(trifluoromethyl)-benzoic acid; 4-(N-isopropylsulfamoyl)-2-(trifluoromethyl)-benzoic acid; 4-(N-cyclopropylsulfamoyl)-2-(trifluoromethyl)-benzoic acid; 4-(N-isopropylsulfamoyl)-2-chloro-benzoic acid; 4-(N-isopropylsulfamoyl)-2-bromo-benzoic acid; and 4-(N-isopropylsulfamoyl)-2-ethyl-benzoic acid.

### Reference Example 12

### 1-Methyl-4-phenyl-1H-pyrrole-2-carboxylic acid

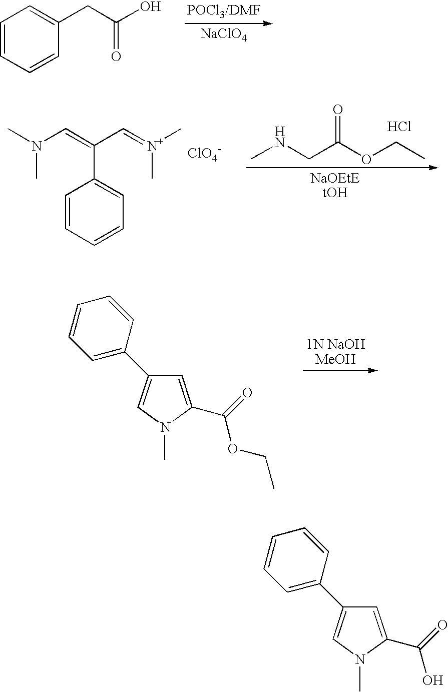

N-[3-(Dimethylamino)-2-phenyl-2-propen-1-ylidene]-N-methylmethanaminium perchlorate. Phenylacetic acid (1.50 g, 11.0 mmol) was added to Vilsmeier's reagent prepared from dimethylformamide (4.25 mL) and phosphoryl oxychloride (3 mL, 33.0 mmol). The reaction was heated to 85° C. and stirred for 13 h. It was cooled to rt and poured into ice-water (50 mL) and sodium perchlorate (1.50 g, 12.2 mmol) was added. The resulting precipitate was filtered, washed with H₂O (25 mL) and diethyl ether (25 mL) to afford N-[3-(Dimethylamino)-2-phenyl-2-propen-1-ylidene]-N-methylmethanaminium perchlorate 2 (2.26 g, 68% yield). ¹H NMR (400 MHz, dmso-d6): δ7.71 (s, 2H), 7.45 (m, 2H), 7.32 (m, 2H), 3.24 (s, 6H), 2.42 (s, 3H).

Ethyl 1-methyl-4-phenyl-1H-pyrrole-2-carboxylate. To a solution of sodium ethoxide (586 mg, 8.27 mmol) in EtOH (100 mL) were added N-[3-(dimethylamino)-2-phenyl-2-propen-1-ylidene]-N-methylmethanaminium perchlorate (1.0 g, 3.31 mmol) and sarcosine ethyl ester HCl salt (763 mg, 4.97 mmol). The resulting mixture was stirred for 24 h under refluxed condition. It was cooled to rt and evaporated solvent in vacuo. H₂O (60 mL) was added to the reaction and extracted with chloroform (200 mL). The organic layer was dried with Na₂SO₄, filtered, concentrated under reduced pressure to afford ethyl 1-methyl-4-phenyl-1H-pyrrole-2-carboxylate (288 mg, 41.0%). ¹H NMR (400 MHz, CDCl₃): δ7.50 (d, 2H), 7.35 (t, 2H), 7.22 (m, 2H), 7.06 (s, 1H), 4.30 (q, 2H), 3.95 (s, 3H), 1.37 (t, 3H).

1-Methyl-4-phenyl-1H-pyrrole-2-carboxylic acid. The reaction mixture of ethyl 1-methyl-4-phenyl-1H-pyrrole-2-carboxylate (288 mg, 1.26 mmol), 1 N NaOH (5 mL) and MeOH (8 mL) was stirred for 3 h at 60° C. The reaction was evaporated under reduced pressure, diluted with H₂O (10 mL) and adjusted pH of reaction to pH 3.0 by the addition of 1 N HCl. It was extracted with EtOAc (20 mL) and the organic layer was dried with Na₂SO₄, filtered, concentrated under reduced pressure to afford 1-methyl-4-phenyl-1H-pyrrole-2-carboxylic acid (201 mg, 79.7% yield). MS (EI) for C₁₂H₁ᵢNO₂, found 202.4 (MH⁺).

### Reference Example 13

### 5-(4-Fluoro-2-methylphenyl)-1-methyl-1H-pyrrole-2-carboxylic acid

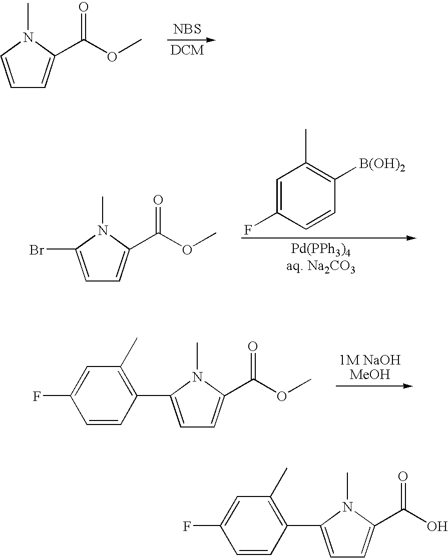

Step 1: Methyl 5-bromo-1-methyl-1H-pyrrole-2-carboxylate. To a dry flask was added methyl 1-methyl-1H-pyrrole-2-carboxylate (6.0 g, 43.1 mmol) and dichloromethane (75 mL), and the flask was wrapped in foil and purged with N₂ for 5 min N-bromosuccimide (8.10 g, 45.5 mmol) was added in one portion and the reaction was stirred for 1 h at rt. The reaction was washed with H₂O (50 mL), sat. NaCl solution (50 mL), dried with Na₂SO₄, filtered, concentrated under reduced pressure and purified by a column chromatography (20% ethylacetate/hexanes) to afford methyl 5-bromo-1-methyl-1H-pyrrol-2-carboxylate (6.70 g, 71.2% yield). MS (EI) for C₇H₈BrNO₂, found 219.2 (MH⁺).

Step 2: Methyl 5-(4-fluoro-2-methylphenyl)-1-methyl-1H-pyrrole-2-carboxylate. Methyl 5-bromo-1-methyl-1H-pyrrol-2-carboxylate (2.0 g, 9.17 mmol) was added to a solution of Pd(PPh₃)₄ (530 mg, 5 mol %) in DME (90 mL). The resulting solution was stirred under N₂ for 5 min. and 4-fluoro-2-methylphenylboronic acid (1.70 g, 11.0 mmol) was added followed by the addition of a solution of Na₂CO₃ (19.5 g, 184 mmol) in H₂O (90 mL). The reaction was stirred for overnight under refluxed condition and allowed to cool to room temperature. It was extracted with dichloromethane (150 mL) and an organic layer was dried over Na₂SO₄, filtered, concentrated under reduced pressure and purified by a column chromatography (20% ethylacetate and hexanes) to afford Methyl 5-(4-fluoro-2-methylphenyl)-1-methyl-1H-pyrrole-2-carboxylate (1.65 g, 72.7% yield). MS (EI) for C₁₄H₁₄FNO₂, found 248.3 (MH⁺).

Step 3: 5-(4-Fluoro-2-methylphenyl)-1-methyl-1H-pyrrole-2-carboxylic acid. The reaction mixture of methyl 5-(4-fluoro-2-methylphenyl)-1-methyl-1H-pyrrole-2-carboxylate (900 mg, 3.64 mmol), 1M NaOH (15 mL) and MeOH (15 mL) was stirred for overnight at 60° C. The reaction was cooled to room temperature and evaporated under reduced pressure, diluted with H₂O (25 mL) and adjusted pH of reaction to pH 3.0 by the addition of 1 N HCl. It was extracted with EtOAc (50 mL) and the organic layer was dried with Na₂SO₄, filtered, concentrated under reduced pressure to afford 5-(4-fluoro-2-methylphenyl)-1-methyl-1H-pyrrole-2-carboxylic acid (720 mg, 84.9% yield). MS (EI) for C₁₃H₁₂FNO₂, found 234.3 (MH⁺).

Using the same or analogous synthetic techniques and substituting with appropriate reagents 5-(pyridin-2-yl)thiophene-2-carboxylic acid, 5-(4-methoxyphenyl)-1-methyl-1H-pyrrole-2-carboxylic acid, 2-phenylthiazole-4-carboxylic acid, 4-(oxazol-5-yl)benzoic acid, and 4-(thiazol-2-yl)benzoic acid were prepared.

Using the same or analogous synthetic techniques, substituting with appropriate reagents, and skipping Step 2,5-bromo-1-methyl-1H-pyrrole-2-carboxylic acid and 5-bromothiophene-2-carboxylic acid were prepared.

### Reference Example 14

### Lithium 1-ethyl-5-methoxy-1H-pyrrolo[3,2-b]pyridine-2-carboxylate

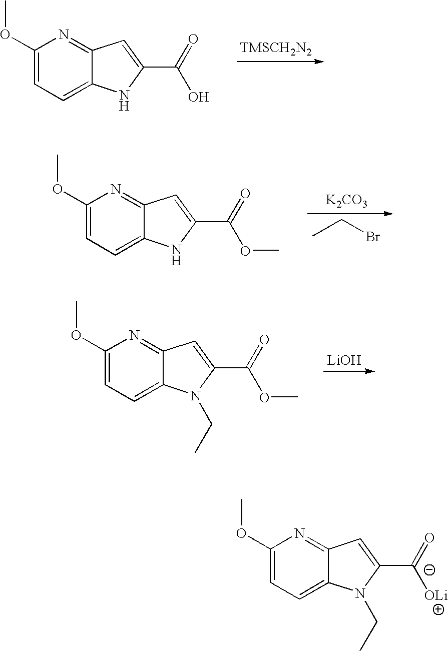

Methyl 5-methoxy-1H-pyrrolo[3,2-b]pyridine-2-carboxylate. 5-methoxy-1H-pyrrolo[3,2-b]pyridine-2-carboxylic acid (0.2 g, 1.04 mmol) was dissolved in THF (1 mL), methanol (0.5 mL, MeOH), followed by (trimethylsilyl)diazomethane (0.025 mL, 1.25 mmol, 2 M in hexanes). After 30 min the reaction was concentrated at reduced pressure affording methyl 5-methoxy-1H-pyrrolo[3,2-b]pyridine-2-carboxylate, the crude product was carried on to the next step without purification. ¹H NMR (400 MHz, CDCl₃); δ 9.12 (s, 1H), 7.68-7.60 (m, 1H), 7.21 (m, 1H), 6.80-6.74 (m, 1H), 4.01 (s, 3H), 3.96 (s, 3H).

Methyl 1-ethyl-5-methoxy-1H-pyrrolo[3,2-b]pyridine-2-carboxylate. To a mixture of methyl 5-methoxy-1H-pyrrolo[3,2-b]pyridine-2-carboxylate (0.215 g, 1.04 mmol) in DMF (10 mL), and K₂CO₃ (0.216 g, 1.56 mmol) was added bromoethane and was stirred at 550° C. over night. The reaction was allowed to cool to room temperature, was diluted with ethyl acetate, transferred to a separatory funnel and the organic phase was washed with water, brine, dried with Na₂SO₄, filtered and concentrated at reduced pressure. The crude was purified by silica column chromatography, eluting with 30% ethyl acetate/hexane to afford 0.160 g (65.5%) of methyl 1-ethyl-5-methoxy-1H-pyrrolo[3,2-b]pyridine-2-carboxylate. ¹H NMR (400 MHz, CDCl₃); δ 7.67-7.60 (m, 1H), 729-7.24 (m, 1H), 6.80-6.74 (m, 1H), 4.64-4.54 (m, 2H), 4.00 (s, 3H), 3.92 (s, 3H), 1.34-1.34 (m, 3H).

Lithium 1-ethyl-5-methoxy-1H-pyrrolo[3,2-b]pyridine-2-carboxylate. Methyl 1-ethyl-5-methoxy-1H-pyrrolo[3,2-b]pyridine-2-carboxylate (0.160 g, 0.683 mmol) was dissolved in a solution of THF/H₂O (6.8 mL, 1:1) and lithium hydroxide (0.030 g, 0.72 mmol) was added. The reaction was stirred at 55 0° C. for 4 h. The reaction was concentrated at reduced pressure and used as such without further purification.

Using the same or analogous synthetic techniques and substituting with appropriate reagents 1-methyl-1H-pyrrolo[2,3-b]pyridine-2-carboxylic acid, 1-ethyl-1H-pyrrolo[2,3-b]pyridine-2-carboxylic acid, 1-ethyl-5-(methylsulfonyl)-1H-indole-2-carboxylic acid, 1-ethyl-5-methoxy-1H-pyrrolo[2,3-c]pyridine-2-carboxylic acid, 1,5-dimethyl-1H-pyrrole-2-carboxylic acid, 1-ethyl-5-methyl-1H-pyrrole-2-carboxylic acid, 2-chloro-4-ethyl-4H-thieno[3,2-b]pyrrole-5-carboxylic acid, 4-(cyanomethyl)-4H-thieno[3,2-b]pyrrole-5-carboxylic acid, and 4-(2-(dimethylamino)-2-oxoethyl)-2-methyl-4H-thieno[3,2-b]pyrrole-5-carboxylic acid were prepared.

### Reference Example 15

### 2-Ethyl-4-(thiazol-2-yl)benzoic acid

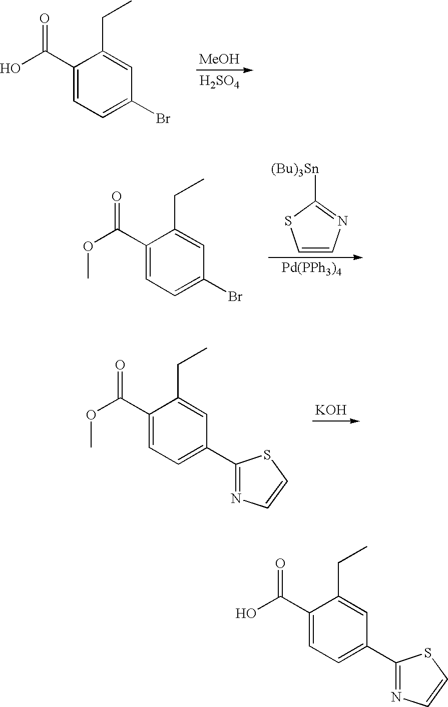

4-Bromo-2-ethylbenzoate. 4-Bromo-2-ethylbenzoic acid (0.457 g, 2 mmol), was dissolved in methanol (60 mL), sulfuric acid (0.18 g, 1.88 mmol) was added and the reaction was allowed to stir at 50° C. for 6 h. The reaction was cooled to room temperature, and concentrated at reduced pressure. The crude was partitioned between ethyl acetate and water, the organic phase was washed with sodium bicarbonate (NaHCO₃, sat), brine, dried with Na₂SO₄, filtered and concentrated at reduced pressure and chromatographed on silica gel to afford methyl 4-bromo-2-ethylbenzoate (0.210 g). ¹H NMR (400 MHz, CDCl₃); δ 7.76-7.70 (m, 1H), 7.46-7.34 (m, H), 3.89 (s, 3H), 3.00-2.90 (m, 2H), 1.28-1.18 (m, 3H).

Methyl 2-ethyl-4-(thiazol-2-yl)benzoate. 4-Bromo-2-ethylbenzoate (0.210 g, 0.86 mmol), was dissolved in toluene (8 mL) under nitrogen. Pd(PPh₃)₄ (0.1 g, 0.087 mmol), was added followed by 2-(tributylstannyl) thiazole (0.646 g, 1.73 mmol) and was heated to reflux. After 25 min. the reaction was cooled to room temperature, potassium fluoride (5 g, 40% wt on alumina, KF) was added and stirred over night. The KF was filtered through celite, washed with KF (1M, 2×), water, brine, dried with Na₂SO₄, filtered, concentrated at reduce pressure and chromatographed on silica gel to afford methyl 2-ethyl-4-(thiazol-2-yl)benzoate (0.145 g). ¹H NMR (400 MHz, CDCl₃); δ7.99-7.78 (m, 4H), 7.43-7.37 (m, 1H), 3.92 (s, 3H), 3.11-3.00 (m, 2H), 1.34-1.23 (m, 3H).

2-Ethyl-4-(thiazol-2-yl)benzoic acid. Methyl 2-ethyl-4-(thiazol-2-yl)benzoate (0.145 g, 0.59 mmol) was dissolved in methanol (6 mL), and 1M potassium hydroxide in water (3.52 mL, 3.52 mmol), was added and the reaction was allowed to stir at 50° C. for 4 h. The was cooled to room temperature; the methanol was removed at reduced pressure, the aqueous phase was acidified with HCl (1 N), was extracted with methanol/dichloromethane (DCM) (8%, 2×), the organic phases were combined, washed with water, brine, dried with Na₂SO₄, filtered and concentrated at reduced pressure affording 2-ethyl-4-(thiazol-2-yl)benzoic acid (0.122 g). MS (EI) 234.0 (MH+).

### Reference Example 16

### 4-{[2-({[(1,1-dimethylethyl)oxy]carbonyl}amino)ethyl]sulfonyl}benzoic acid

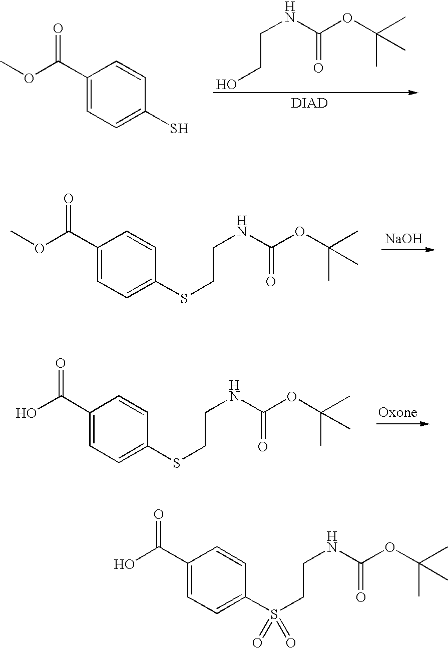

Methyl 4-{[2-({[(1,1-dimethylethyl)oxy]carbonyl}amino)ethyl]thio}benzoate. Diisopropyl azodicarboxylate (0.4 mL, 1.93 mmol) was dissolved in THF and was cooled to 0° C. and triphenylphosphine (0.505 g, 1.93 mmol) was added. The mixture was stirred for 1 h and then tert-butyl N-(2-hydroxyethyl)-carbamate (0.3 mL, 1.94 mml) was added. After 0.3 h, methyl 4-mercaptobenzoate (0.326 g, 1.94 mmol) dissolved in THF (1 mL) was added to the mixture and after 20 h at ambient, the mixture was concentrated. The residue was dissolved in ethyl acetate and washed sequentially with 0.5N HCl, saturated sodium bicarbonate and brine. The organic portion was dried over sodium sulfate, filtered, was concentrated and the residue was purified by flash chromatography (1-20% ethyl acetate in hexanes) to afford methyl 4-{[2-({[(1,1-dimethylethyl)oxy]carbonyl}amino)ethyl]thio}benzoate (0.432 g, 1.39 mmol, 72% yield). ¹H NMR (400 MHz, CDCl₃): δ 7.94 (d, 2H), 7.35 (d, 2H), 4.90 (br s, 1H), 3.91 (s, 3H), 3.40 (q, 2H), 3.14 (t, 2H), 1.44 (s, 9H). MS (EI) for C₁₅H₂₁NO₄S: 212.1 (M-Boc)H⁺.

4-{[2-({[(1,1-Dimethylethyl)oxy]carbonyl}amino)ethyl]thio}benzoic acid. Methyl 4-{[2-({[(1,1-dimethylethyl)oxy]carbonyl}amino)ethyl]thio}benzoate (0.409 g, 1.32 mmol) was dissolved in methanol (4 mL) and dichloromethane (2 mL) and was treated with 1 M NaOH (3 mL) at ambient for 0.75 h and then at 45° C. for 2 h. The mixture was concentrated and the aqueous residue was acidified with 1 N HCl to pH˜2. The precipitate which formed was collected by filtration, washed with water and was dried to afford 4-{[2-({[(1,1-dimethylethyl)oxy]carbonyl}amino)ethyl]thio}benzoic acid as a colorless solid (0.374 g, 1.26 mmol, 95% yield). ¹H NMR (400 MHz, d₆-DMSO): δ 12.9 (br s, 1H), 7.84 (d, 2H), 7.40 (d, 2H), 7.09 (t, 1H), 3.20-3.12 (m, 2H), 3.11-3.05 (m, 2H), 1.38 (s, 9H). MS (EI) for C₁₄H₁₉NO₄S: 296.0 (M−H).

4-{[2-({[(1,1-Dimethylethyl)oxy]carbonyl}amino)ethyl]sulfonyl}benzoic acid. 4-{[2-({[(1,1-Dimethylethyl)oxy]carbonyl}amino)ethyl]thio}benzoic acid (0.374 g, 1.26 mmol) was dissolved in 1N NaOH (1.3 mL). Water (5 mL), acetone (1 mL) and sodium bicarbonate (0.316 g, 3.76 mmol) were added, followed by careful portionwise addition of Oxone (1 g, 1.63 mmol). After stirring for 0.25 h, the mixture was diluted with ethyl acetate and was acidified with 3N HCl (2 mL). The aqueous portion was extracted with ethyl acetate. The combined organic portion was washed with brine, was dried over sodium sulfate, filtered and was concentrated to afford 4-{[2-({[(1,1-dimethylethyl)oxy]carbonyl}amino)ethyl]sulfonyl}benzoic acid as a colorless solid (0.407 g, 1.24 mmol, 98% yield). ¹H NMR (400 MHz, d₆-DMSO): δ 13.6 (br s, 1H), 8.17 (d, 2H), 8.02 (d, 2H), 6.85 (t, 1H), 3.48 (t, 2H), 3.21 (q, 2H), 1.28 (s, 9H). MS (EI) for C₁₄H₁₉NO₆S: 328.0 (M−H).

### Reference Example 17

### 2-Methyl-4-(trifluoroacetyl)benzoic acid and 4-bromo-2-methylbenzoic acid

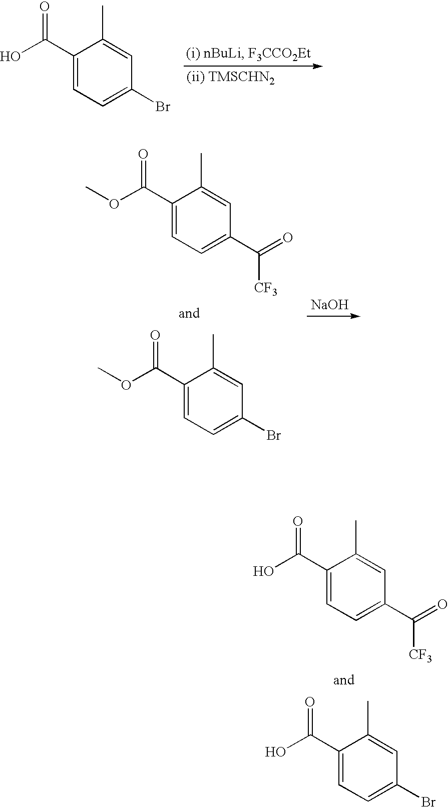

Methyl 2-methyl-4-(trifluoroacetyl)benzoate and methyl 4-bromo-2-methylbenzoate. 4-Bromo-2-methylbenzoic acid 1 (0.43 g, 2 mmol) was dissolved in THF (20 mL) and was cooled to −78° C. n-Butyllithium (2.5 M in hexanes; 1.6 mL, 4 mmol) was added dropwise and the mixture was stirred at −78° C. for 0.3 h. Ethyl trifluoroacetate (0.24 mL, 2 mmol) was added dropwise and the mixture was stirred at −78° C. for a further 0.25 h. 1 N HCl (2 mL) was added, the mixture was warmed to ambient and further 1N HCl (5 mL) was added. The mixture was extracted with ethyl acetate (2×). The combined organic portion was washed with brine, dried over sodium sulfate, filtered and concentrated to afford a yellow oil which was purified by preparative reverse phase HPLC(CH₃CN/H₂O). Acetonitrile was removed from the isolated pure fractions on a rotary evaporator and the aqueous remainder was lyophilized to give a pale brown solid which was directly dissolved in methanol (10 mL) and THF (20 mL) and treated with (trimethylsilyl)diazomethane solution (2.0M in hexanes, 1.5 mL). The mixture was concentrated and the residue was purified by flash chromatography (20-40% ethyl acetate in hexanes) to afford a mixture of ˜80% methyl 2-methyl-4-(trifluoroacetyl)benzoate and ˜20% methyl 4-bromo-2-methylbenzoate as a yellow oil (140 mg). GCMS (EI) for C₁₁H₉F₃O₃: 246 (M⁺).

2-Methyl-4-(trifluoroacetyl)benzoic acid and 4-bromo-2-methylbenzoic acid. The mixture of ˜80% methyl 2-methyl-4-(trifluoroacetyl)benzoate and ˜20% methyl 4-bromo-2-methylbenzoate (140 mg) was dissolved in methanol (2 mL) and was treated with 1 M NaOH (1.5 mL) at 45° C. for 1 h. The mixture was concentrated, the aqueous residue was washed with ether and then was acidified with 1 N HCl to pH˜1 and was extracted with ethyl acetate (2×). The combined organic extract was dried over sodium sulfate, filtered and concentrated to afford a mixture of 2-methyl-4-(trifluoroacetyl)benzoic acid and 4-bromo-2-methylbenzoic acid as a colorless solid (104 mg). MS (EI) for C₁₀H₇F₃O₃: 231.0 (M−H).

### Reference Example 18

### 3-Methyl-5-(methylsulfonyl)thiophene-2-carboxylic acid

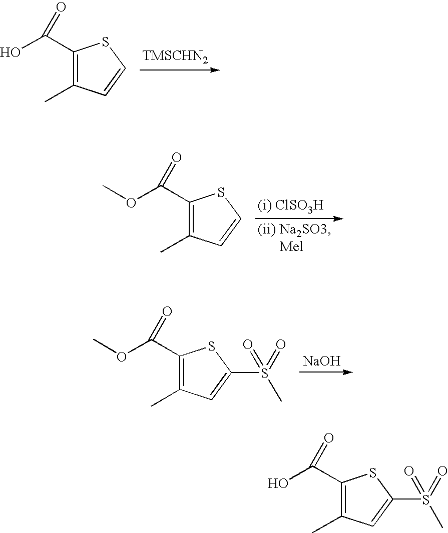

Methyl 3-methylthiophene-2-carboxylate. 3-Methylthiophene-2-carboxylic acid (1.42 g, 10 mmol) was dissolved in methanol (6 mL) and THF (20 mL) and was cooled in an ice bath. The mixture was treated with (trimethylsilyl)diazomethane solution (2.0M in hexanes, 6 mL). The mixture was concentrated and the residue was purified by flash chromatography (5-15% diethyl ether in hexanes) to afford the product as a colorless oil (1.19 g, 7.63 mmol, 76% yield). ¹H NMR (400 MHz, CDCl₃): δ 7.39 (d, 1H), 6.92 (d, 1H), 3.86 (s, 3H), 2.56 (s, 3H). GCMS (EI) for C₇H₈O₂S: 156 (M⁺).

Methyl 3-methyl-5-(methylsulfonyl)thiophene-2-carboxylate. A solution of chlorosulfonic acid (0.4 mL, 0.602 mmol) in anhydrous chloroform (5 mL) was cooled in an ice bath. Methyl 3-methylthiophene-2-carboxylate (312 mg, 2 mmol) was dissolved in anhydrous chloroform (2 mL) and was added dropwise to the cooled chlorosulfonic acid solution. The mixture was stirred for 1 h and then was warmed to ambient and was stirred for a further 5 h. The mixture was poured into ice-water (10 mL) and was extracted with ethyl acetate (2×). The combined organic portion was washed with brine, dried over sodium sulfate, filtered and was concentrated to afford a colorless solid (114 mg) which was suspended in 1,4-dioxane (2 mL) and water (2 mL) and was treated with sodium sulfite (113 mg, 0.897 mmol) and sodium bicarbonate (75 mg, 0.892 mmol) at 90° C. for 0.5 h. The mixture was concentrated and the residue was suspended in DMF (2 mL) and was treated with iodomethane (0.1 mL) at ambient for 1 h. The mixture was quenched with 5% lithium chloride solution and was extracted with ethyl acetate (2×). The combined organic portion was washed with brine, dried over sodium sulfate, filtered and was concentrated to afford a yellow oil which was purified by flash chromatography (15-30% ethyl acetate in hexanes) to afford the product as a colorless solid (18 mg, 0.077 mmol, 4% yield). ¹H NMR (400 MHz, CDCl₃): δ 8.28 (s, 1H), 3.91 (s, 3H), 3.10 (s, 3H), 2.80 (s, 3H). GCMS (EI) for C₈H₁₀O₄S₂: 234 (M⁺).

3-Methyl-5-(methylsulfonyl)thiophene-2-carboxylic acid. Methyl 3-methyl-5-(methylsulfonyl)thiophene-2-carboxylate (17 mg, 0.073 mmol) was dissolved in methanol (0.5 mL) and dichloromethane (0.5 mL) and was treated with 1 N NaOH (0.2 mL) at 50° C. for 1 h and then at ambient for 15 h. The mixture was concentrated, the aqueous residue was washed with ether and then was acidified with 1 N HCl to pH˜1 and was extracted with ethyl acetate (2×). The combined organic extract was dried over sodium sulfate, filtered and concentrated to afford 3-methyl-5-(methylsulfonyl)thiophene-2-carboxylic acid as a colorless solid (14 mg, 0.064 mmol, 87% yield). MS (EI) for C₇H₈O₄S₂: 219.0 (M−H).

### Reference Example 19

### 4-Iodo-2-methylbenzoic acid

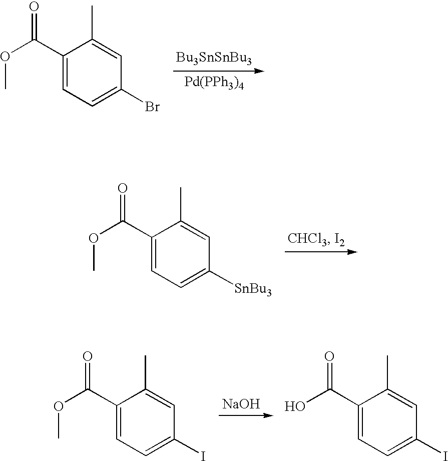

Methyl 2-methyl-4-(tributylstannanyl)benzoate. Methyl 4-bromo-2-methylbenzoate (1 g, 4.37 mmol) was dissolved in anhydrous toluene (100 mL) and bis(tri-n-butyltin) (6.5 mL, 12.9 mmol) was added. The mixture was sparged with nitrogen for 10 minutes and then tetrakis(triphenylphosphine)palladium(0) (0.255 g, 0.221 mmol) and triethylamine (1.2 mL, 8.60 mmol) were added. The mixture was stirred at 95° C. for 4 h and then at ambient for 40 h. The mixture was concentrated and the residue was purified by flash chromatography (1-10% ethyl acetate in hexanes) to afford the product as a colorless oil (1.315 g, 3.08 mmol, 70% yield). ¹H NMR (400 MHz, CDCl₃): δ 7.82 (d, 1H), 7.41-7.30 (m, 2H), 3.88 (s, 3H), 2.58 (s, 3H), 1.66-1.43 (m, 6H), 1.41-1.22 (m, 6H), 1.15-0.97 (m, 6H), 0.88 (t, 9H).

Methyl 4-iodo-2-methylbenzoate. Methyl 2-methyl-4-(tributylstannanyl)benzoate (600 mg, 1.41 mmol) was dissolved in chloroform (15 mL). Iodine (670 mg, 2.64 mmol) was dissolved in chloroform (10 mL) and was added to the reaction mixture and was stirred for 10 minutes. The reaction was quenched by stirring with saturated sodium bisulfite solution (45 mL) and after 5 minutes was extracted with chloroform. The organic portion was dried over sodium sulfate, filtered and concentrated. The residue was purified by flash chromatography (1-10% ethyl acetate in hexanes) to afford the product as a colorless oil.

4-Iodo-2-methylbenzoic acid. The methyl 4-iodo-2-methylbenzoate from above was dissolved in methanol (4 mL) and dichloromethane (2 mL) and treated with 1 N NaOH (3 mL) at 45° C. for 3 h and then at 60° C. for 15 h. The mixture was concentrated, the aqueous residue was washed with ether and then was acidified with 1 N HCl to pH˜1 and was extracted with ethyl acetate (2×). The combined organic extract was dried over sodium sulfate, filtered and concentrated to afford the product as an off-white solid (313 mg, 1.19 mmol, 84% yield). ¹H NMR (400 MHz, d₆-DMSO): δ 13.0 (br s, 1H), 7.74 (s, 1H), 7.67 (dd, 1H), 7.57 (s, 1H), 2.47 (s, 3H). MS (EI) for C₈H₇IO₂: 260.9 (M−H).

### Reference Example 20

### 5-(Methoxycarbonyl)-1H-pyrrole-2-carboxylic acid

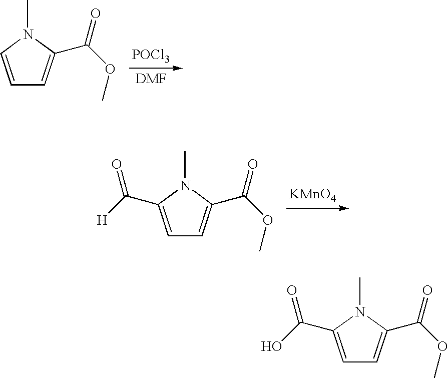

Methyl 5-formyl-1-methyl-1H-pyrrole-2-carboxylate. Phosphorus oxychloride (4.9 mL, 52.6 mmol, POCl₃) was added to a 100 mL round bottom flask; dimethylformide (2.3 mL, 29.6 mmol, DMF) was added over 15 minutes, followed by dichloroethane (10 mL, DCE) and was cooled to 0° C. Methyl 1-methyl-1H-pyrrole-2-carboxylate (3.62 g, 26.0 mmol) was mixed with DCE (10 mL) and was added to the reaction over 1 h. The reaction was heated to reflux for 15 min, and then it was cooled to room temperature. Sodium acetate (13.1 g, 160 mmol) was dissolved in water (30 mL) and was added to the reaction over 5 min. Diethyl ether was added; the mixture was transferred to a separatory funnel, the aqueous phase was separated, and extracted with diethyl ether. The organic phases were combined, washed with sodium bicarbonate (NaHCO₃, sat), dried with anhydrous sodium sulfate (Na₂SO₄), filtered and concentrated at reduced pressure. The crude was purified by silica column chromatography, eluting from 10-15% ethyl acetate/hexane to afford 1.07 g (24.5% yield) of the product. ¹H NMR (400 MHz, CDCl₃); δ 9.23 (s, 1H), 6.96-6.85 (m, 2H), 4.29 (s, 3H), 3.88 (s, 3H).

5-(Methoxycarbonyl)-1H-pyrrole-2-carboxylic acid. Methyl 5-formyl-1-methyl-1H-pyrrole-2-carboxylate (1.07 g, 6.38 mmol) was dissolved in acetone (150 mL), potassium permanganate (2.02 g, 12.8 mmol, KMnO₄) was dissolved in acetone/water (1:1, 250 mL) and was added to 2 over two hours, the reaction was allowed to stir for 3 h. Then the reaction was poured into sodium sulfite (10%, in 1 N hydrochloric acid, 250 mL, HCl). The mixture was transferred to a separatory funnel and was extracted with chloroform (CHCl₃ 3×200 mL), the organic phases were combined washed with water, and sodium bicarbonate (5%, 3×200 mL, NaHCO₃). The NaHCO₃ phases were combined, acidified with HCl (2 N), and extracted with CHCl₃ (3×200 mL). The organic phases were combined dried with Na₂SO₄, filtered, and concentrated at reduced pressure to afford 0.928 g of the product. ¹H NMR (400 MHz, CDCl₃); δ 7.08-6.88 (m, 2H), 4.29 (s, 3H), 3.87 (s, 3H).

### Reference Example 21

### 4-[(3-Bromopropyl)sulfonyl]benzoic acid

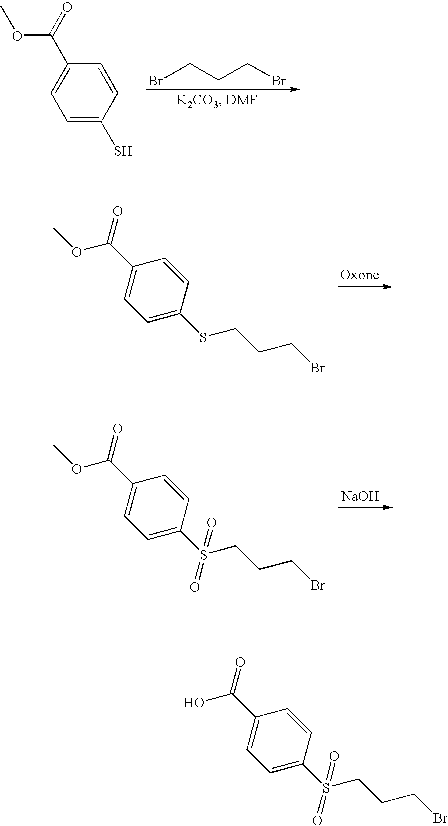

Methyl 4-(3-bromopropylthio)benzoate. Methyl 4-mercaptobenzoate (500 mg, 2.98 mmol) was dissolved in DMF (8 mL) and 1,3-dibromopropane (1.5 mL, 14.8 mmol) and potassium carbonate (411 mg, 2.98 mmol) were added. The mixture was stirred at ambient for 15 h and then was partitioned between ethyl acetate and brine. The organic portion was dried over sodium sulfate, filtered and was concentrated to afford the product as a colorless oil which was used without further purification.

Methyl 4-[(3-bromopropyl)sulfonyl]benzoate. The methyl 4-(3-bromopropylthio)benzoate from above was dissolved in acetone: methanol:water (8:8:5; 21 mL) and was treated with Oxone (5 g, 8.13 mmol) at ambient for 2 h. The mixture was diluted with chloroform, was washed with water and brine and was dried over sodium sulfate, filtered and was concentrated to afford a pale yellow semi-solid which was triturated with ethyl acetate and filtered. The filtrate was concentrated and was purified by flash chromatography (10-40% ethyl acetate in hexanes) to afford the product as colorless crystals (722 mg, 2.25 mmol, 75% yield). ¹H NMR (400 MHz, CDCl₃): δ 8.25 (d, 2H), 8.00 (d, 2H), 3.98 (s, 3H), 3.48 (t, 2H), 3.30-3.27 (m, 2H), 2.36-2.27 (m, 2H). MS (EI) for C₁₁H₁₃BrO₄S: 337.9, 339.9 (M+H₂O)⁺Br isotope pattern.

4-[(3-Bromopropyl)sulfonyl]benzoic acid. Methyl 4-[(3-bromopropyl)sulfonyl]benzoate (515 mg, 1.60 mmol) was dissolved in methanol (4 mL) and dichloromethane (2 mL) and was treated with 1 N NaOH (3.5 mL) at ambient for 3 h. The mixture was concentrated, the aqueous residue was washed with ether and then was acidified with 1 N HCl to pH˜1 and was extracted with ethyl acetate (2×). The combined organic extract was dried over sodium sulfate, filtered and concentrated to afford the product as a colorless solid (467 mg, 1.52 mmol, 95% yield). ¹H NMR (400 MHz, d₆-DMSO): δ 13.6 (s, 1H), 8.19 (d, 2H), 8.04 (d, 2H), 3.55 (t, 2H), 3.52-3.45 (m, 2H), 2.12-2.02 (m, 2H). MS (EI) for C₁₀H₁₁BrO₄S: 323.9, 325.9 (M+H₂O)⁺ and 304.9, 306.9 (M−H) Br isotope pattern.

### Reference Example 22

### 2-Ethyl-4-(thiazol-2-yl)benzoic acid

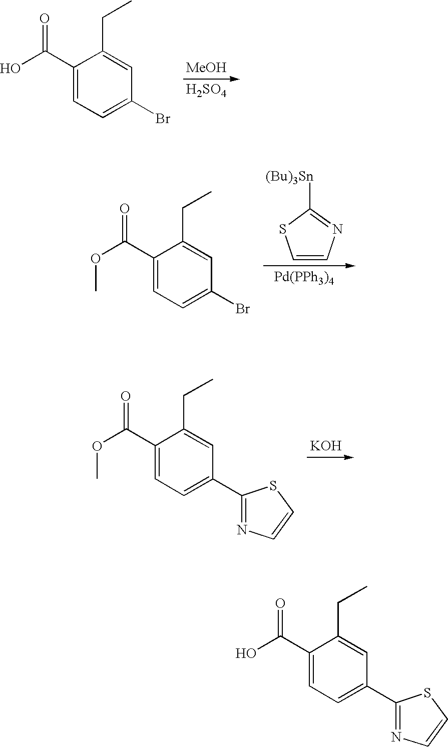

Methyl 4-bromo-2-ethylbenzoate. 4-Bromo-2-ethylbenzoic acid (0.457 g, 2 mmol), was dissolved in methanol (60 mL), sulfuric acid (0.18 g, 1.88 mmol) was added and the reaction was allowed to stir at 50° C. for 6 h. The reaction was cooled to room temperature, and concentrated at reduced pressure. The crude was partitioned between EA and water, the organic phase was washed with sodium bicarbonate (NaHCO₃, sat), brine, dried with Na₂SO₄, filtered and concentrated at reduced pressure and chromatographed on silica gel to afford methyl 4-bromo-2-ethylbenzoate (0.210 g). ¹H NMR (400 MHz, CDCl₃); δ 7.76-7.70 (m, 1H), 7.46-7.34 (m, H), 3.89 (s, 3H), 3.00-2.90 (m, 2H), 1.28-1.18 (m, 3H).

Methyl 2-ethyl-4-(thiazol-2-yl)benzoate. 4-Bromo-2-ethylbenzoate (0.210 g, 0.86 mmol), was dissolved in toluene (8 mL) under nitrogen. Pd(PPh₃)₄ (0.1 g, 0.087 mmol), was added followed by 2-(tributylstannyl) thiazole (0.646 g, 1.73 mmol) and was heated to reflux. After 25 min. the reaction was cooled to room temperature, potassium fluoride (5 g, 40% wt on alumina, KF) was added and stirred over night. The KF was filtered through celite, washed with KF (1M, 2×), water, brine, dried with Na₂SO₄, filtered, concentrated at reduce pressure and chromatographed on silica gel to afford methyl 2-ethyl-4-(thiazol-2-yl)benzoate (0.145 g). ¹H NMR (400 MHz, CDCl₃); δ7.99-7.78 (m, 4H), 7.43-7.37 (m, 1H), 3.92 (s, 3H), 3.11-3.00 (m, 2H), 1.34-1.23 (m, 3H).

2-Ethyl-4-(thiazol-2-yl)benzoic acid. Methyl 2-ethyl-4-(thiazol-2-yl)benzoate (0.145 g, 0.59 mmol) was dissolved in methanol (6 mL), and potassium hydroxide (3.52 mL, 3.52 mmol, 1 M in water, KOH), was added and the reaction was allowed to stir at 50° C. for 4 h. The was cooled to room temperature; the methanol was removed at reduced pressure, the aqueous phase was acidified with HCl (1 M), was extracted with methanol/dichloromethane (DCM) (8%, 2×), the organic phases were combined, washed with water, brine, dried with Na₂SO₄, filtered and concentrated at reduced pressure affording 2-ethyl-4-(thiazol-2-yl)benzoic acid (0.122 g). MS (EI) 234.0 (MH+).

Using the same or analogous synthetic techniques and substituting with appropriate reagents 2-methyl-4-(thiazol-2-yl)benzoic acid was prepared.

### Reference Example 23

### 4-(2-(tert-butoxycarbonylamino)ethylsulfonyl)benzoic acid

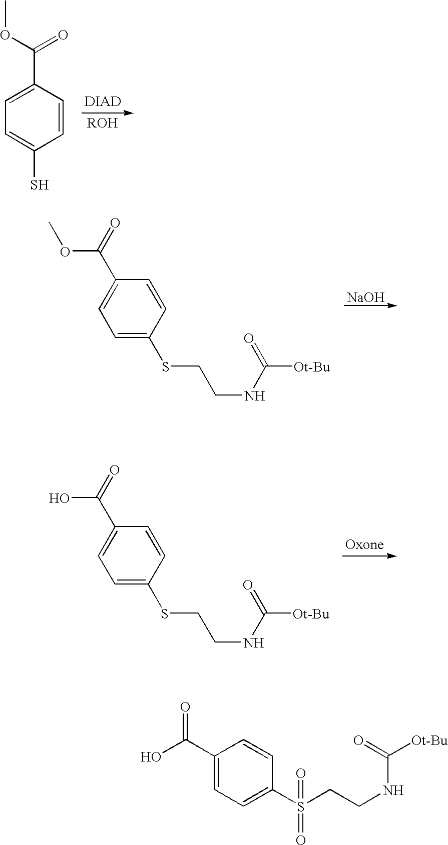

Diisopropyl azodicarboxylate (0.4 mL, 1.93 mmol) was dissolved in THF and was cooled to 0° C. and triphenylphosphine (0.505 g, 1.93 mmol) was added. The mixture was stirred for 1 h and then tert-butyl-N-(2-hydroxyethyl)-carbamate (0.3 mL, 1.94 mml) was added. After 0.3 h, methyl 4-mercaptobenzoate (0.326 g, 1.94 mmol) dissolved in THF (1 mL) was added to the mixture and after 20 h at ambient, the mixture was concentrated. The residue was dissolved in ethyl acetate and washed sequentially with 0.5N HCl, saturated sodium bicarbonate and brine. The organic portion was dried over sodium sulfate, filtered, was concentrated and the residue was purified by flash chromatography (1-20% ethyl acetate in hexanes) to afford methyl 4-{[2-({[(1,1-dimethylethyl)oxy]carbonyl}amino)ethyl]thio}benzoate (0.432 g, 1.39 mmol, 72% yield). ¹H NMR (400 MHz, CDCl₃): δ 7.94 (d, 2H), 7.35 (d, 2H), 4.90 (br s, 1H), 3.91 (s, 3H), 3.40 (q, 2H), 3.14 (t, 2H), 1.44 (s, 9H). MS (EI) for C₁₅H₂₁NO₄S: 212.1 (M-Boc)H⁺.

Methyl 4-{[2-({[(1,1-dimethylethyl)oxy]carbonyl}amino)ethyl]thio}benzoate (0.409 g, 1.32 mmol) was dissolved in methanol (4 mL) and dichloromethane (2 mL) and was treated with 1 N NaOH (3 mL) at ambient for 0.75 h and then at 45° C. for 2 h. The mixture was concentrated and the aqueous residue was acidified with 1N HCl to pH˜2. The precipitate which formed was collected by filtration, washed with water and was dried to afford 4-{[2-({[(1,1-dimethylethyl)oxy]carbonyl}amino)ethyl]thio}benzoic acid as a colorless solid (0.374 g, 1.26 mmol, 95% yield). ¹H NMR (400 MHz, d₆-DMSO): δ 12.9 (br s, 1H), 7.84 (d, 2H), 7.40 (d, 2H), 7.09 (t, 1H), 3.20-3.12 (m, 2H), 3.11-3.05 (m, 2H), 1.38 (s, 9H). MS (EI) for C₁₄H₁₉NO₄S: 296.0 (M−H).

4-{[2-({[(1,1-Dimethylethyl)oxy]carbonyl}amino)ethyl]thio}benzoic acid (0.374 g, 1.26 mmol) was dissolved in 1N NaOH (1.3 mL). Water (5 mL), acetone (1 mL) and sodium bicarbonate (0.316 g, 3.76 mmol) were added, followed by careful portionwise addition of Oxone (1 g, 1.63 mmol). After stirring for 0.25 h, the mixture was diluted with ethyl acetate and was acidified with 3N HCl (2 mL). The aqueous portion was extracted with ethyl acetate. The combined organic portion was washed with brine, was dried over sodium sulfate, filtered and was concentrated to afford 4-{[2-({[(1,1-dimethylethyl)oxy]carbonyl}amino)ethyl]sulfonyl}benzoic acid as a colorless solid (0.407 g, 1.24 mmol, 98% yield). ¹H NMR (400 MHz, d₆-DMSO): δ 13.6 (br s, 1H), 8.17 (d, 2H), 8.02 (d, 2H), 6.85 (t, 1H), 3.48 (t, 2H), 3.21 (q, 2H), 1.28 (s, 9H). MS (EI) for C₁₄H₁₉NO₆S: 328.0 (M−H).

Using the same or analogous synthetic techniques and substituting with appropriate reagents 4-(2-(tetrahydro-2H-pyran-2-yloxy)ethylsulfonyl)benzoic acid were and 4-(2-hydroxyethylsulfonyl)benzoic acid prepared.

### Example 1

### N-Methyl-4-[(7-quinolin-3-yl-2,3-dihydro-1,4-benzoxazepin-4(5H)-yl)carbonyl]benzamide

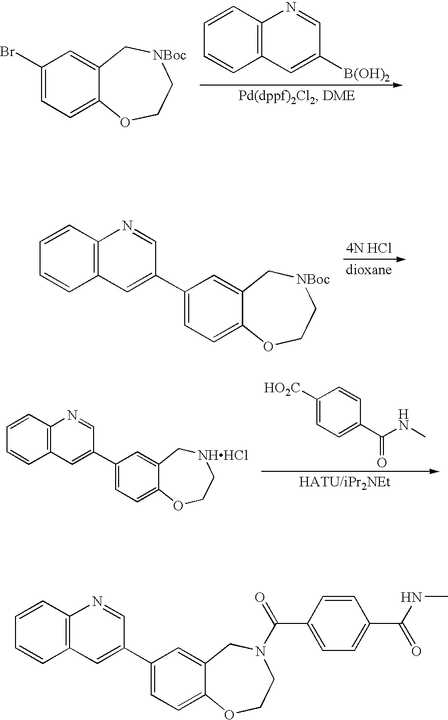

tert-Butyl 7-(quinoline-3-yl)-2,3-dihydrobenzo[f][1,4]oxazepine-4(5H)-carboxylate. A mixture of tert-butyl 7-bromo-2,3-dihydrobenzo[f][1,4]-oxazepine-4(5H)-carboxylate (3.27 g, 10 mmol), prepared as described in Reference Example 4, quinoline-3-yl boronic acid (2.00 g, 11.5 mmol), Pd(dppf)₂Cl₂ (10 mol %), and potassium carbonate (4.14 g, 30 mmol) in DME (50 mL) was heated at 80° C. for 2 h. The reaction was cooled to rt. Water (50 mL) and EtOAc (100 mL) was added. The organic layer was washed with brine (50 mL), dried with Na₂SO₄, filtered, concentrated and chromatographed on silica gel to afford the product (2.82 g, 70%) as a white solid. MS (EI) for C₂₂H₂₄N₂O₃, found 377 (MH⁺).

7-(Quinolin-3-yl)-2,3-dihydrobenzo[f][1,4]oxazepine hydrochloride. A solution of tert-butyl 7-(quinoline-3-yl)-2,3-dihydrobenzo[f][1,4]oxazepine-4(5H)-carboxylate (2.6 g, 6.9 mmol) and 4 N hydrogen chloride in dioxane (15 mL) was stirred at 60° C. for 1 h. The resulting white solid was collected by filtration and washed with diethyl ether to give the desired product as a white solid (1.7 g).

N-Methyl-4-[(7-quinolin-3-yl-2,3-dihydro-1,4-benzoxazepin-4(5H)-yl)carbonyl]benzamide. 4-(Methylcarbamoyl)benzoic acid (89.5 mg, 0.5 mmol), Hunig's base (0.17 mL, 1.0 mmol), and HATU (202 mg, 0.53 mmol) were combined in DMF (1.0 mL) to which was added a solution of 7-(quinoline-3-yl)-2,3-dihydrobenzo[f][1,4]oxazepine hydrochloride (156 mg, 0.5 mmol) and Hunig's base (0.52 mL, 3 mmol) in DMF (2.0 mL). The resulting reaction mixture was stirred at 50° C. for 20 min then diluted with aqueous 5% NaHCO₃. The resulting solids were filtered, washed with H₂O and purified by preparative HPLC to give N-methyl-4-[(7-quinolin-3-yl-2,3-dihydro-1,4-benzoxazepin-4(5H)-yl)carbonyl]benzamide (68 mg, 31%) as a white solid. ¹H NMR (400 MHz, DMSO-d₆): δ 9.28-7.07 (m, 14H), 4.85 (s, 1H), 4.61 (s, 1H), 4.38 (s, 1H), 4.21 (s, 1H), 4.04 (s, 1H), 3.77 (s, 1H), 3.36 (s, 3H). MS (EI) for C₂₇H₂₃N₃O₃, found 438 (MH+).

Using the same or analogous synthetic techniques and substituting with appropriate reagents, the following examples were prepared.

7-Quinolin-3-yl-4-{[4-(1,2,3-thiadiazol-4-yl)phenyl]carbonyl}-2,3,4,5-tetrahydro-1,4-benzoxazepine. ¹H NMR (400 MHz, DMSO-d₆): δ 9.7 (s, 1H), 9.40-7.41 (m, 10H), 7.21 (m, 2H), 4.95 (s, 1H), 4.67-3.86 (m, 4H), 3.59 (s, 1H). MS (EI) for C₂₇H₂₀N₄OS, found 465 (MH+).

1,1,1,3,3,3-Hexafluoro-2-{4-[(7-quinolin-3-yl-2,3-dihydro-1,4-benzoxazepin-4(5H)-yl)carbonyl]phenyl}propan-2-ol. ¹H NMR (400 MHz, DMSO-d₆): δ 9.3 (s, 1H), 9.11-6.90 (m, 12H), 4.94 (s, 1H), 4.57-3.7 (m, 5H). MS (EI) for C₂₈H₂O F₆N₄O₃, found 546 (MH+).

4-{[4-(1-Methyl-1H-pyrazol-4-yl)phenyl]carbonyl}-7-quinolin-3-yl-2,3,4,5-tetrahydro-1,4-benzoxazepine. ¹H NMR (400 MHz, DMSO-d₆): δ 9.31-7.12 (m, 15H), 4.91 (s, 1H), 4.77-3.91 (m, 5H), 3.5 (s, 3H), 3.72 (s, 1H). MS (EI) for C₂₉H₂₄N₄O₂, found 461 (MH+).

4-({4-[1-(1-Methylethyl)-1H-pyrazol-4-yl]phenyl}carbonyl)-7-quinolin-3-yl-2,3,4,5-tetrahydro-1,4-benzoxazepine. ¹H NMR (400 MHz, DMSO-d₆): δ 9.30-7.18 (m, 15H), 4.89 (s, 1H), 4.71 (m, 6H), 1.49 (m, 6H). MS (EI) for C₃ᵢH₂₈N₄O₂, found 489 (MH+).

4-{[4-(5-Ethyl-1,2,4-oxadiazol-3-yl)phenyl]carbonyl}-7-quinolin-3-yl-2,3,4,5-tetrahydro-1,4-benzoxazepine. ¹H NMR (400 MHz, DMSO-d₆): δ 9.18 (d, 1H), 8.51 (d, 1H), 8.13-7.76 (m, 7H), 7.67 (t, 1H), 7.57-7.08 (m, 3H), 4.93 (s, 1H), 4.60 (s, 1H), 4.35 (s, 1H), 4.22 (s, 1H), 4.01 (s, 1H), 3.77 (s, 1H), 2.52 (q, 2H), 1.28 (t, 3H). MS (EI) for C₂₉H₂₄N₄O₃, found 478 (MH+).

N,N-Dimethyl-4-[(7-quinolin-3-yl-2,3-dihydro-1,4-benzoxazepin-4(5H)-yl)carbonyl]benzamide. ¹H NMR (400 MHz, DMSO-d₆): δ 9.28-7.07 (m, 13H), 4.85 (s, 1H), 4.61 (s, 1H), 4.38 (s, 1H), 4.23 (s, 1H), 4.04 (s, 1H), 3.80 (s, 1H), 2.89 (m, 6H). MS (EI) for C₂₈H₂₅N₃O₃, found 452 (MH+).

4-[(7-Quinolin-3-yl-2,3-dihydro-1,4-benzoxazepin-4(5H)-yl)carbonyl]-N-(2,2,2-trifluoroethyl)benzamide. ¹H NMR (400 MHz, DMSO-d₆): δ 9.24-7.17 (m, 14H), 4.92 (s, 1H), 4.60 (s, 1H), 4.38 (s, 1H), 4.2 (s, 1H), 4.17-4.02 (m, 4H). MS (EI) for C₂₈H₂₂F₃N₃O₃, found 506 (MH+).

7-(1-Benzothien-2-yl)-4-{[2-ethyl-4-(methylsulfonyl)phenyl]carbonyl}-2,3,4,5-tetrahydro-1,4-benzoxazepine. ¹H NMR (400 MHz, DMSO-d₆): δ 8.01-6.61 (m, 11H), 4.92 (m, 1H), 4.43-4.03 (m, 4H), 3.58 (s, 1H), 3.31 (s, 3H), 2.63-2.18 (m, 4H), 1.02 and 1.18 (t, 3H). MS (EI) for C₂₇H₂₅NO₄S, found 492 (MH+).

7-(1-Benzothien-2-yl)-4-{[3-fluoro-2-methyl-4-(methylsulfonyl)phenyl]carbonyl}-2,3,4,5-tetrahydro-1,4-benzoxazepine. ¹H NMR (400 MHz, DMSO-d₆): δ 7.98-6.81 (m, 10H), 4.91 (m, 1H), 4.51-3.94 (m, 4 h), 3.6 (s, 1H), 3.32 (s, 3H), 1.81 and 2.08 (s, 3H). MS (EI) for C₂₆H₂₂FNO₄S₂, found 496 (MH+).

7-(1-Benzothien-2-yl)-4-{[2-bromo-4-(methylsulfonyl)phenyl]carbonyl}-2,3,4,5-tetrahydro-1,4-benzoxazepine. ¹H NMR (400 MHz, DMSO-d₆): δ 8.24-6.76 (m, 11H), 4.89 (m, 1H), 4.51-4.01 (m, 4H), 3.58 (s, 1H), 3.40 (s, 3H). MS (EI) for C₂₅H₂₀BrNO₄S₂, found 543 (MH+).

2,2,2-Trifluoro-1-(4-{[7-(1-methyl-1H-pyrazol-4-yl)-2,3-dihydro-1,4-benzoxazepin-4(5H)-yl]carbonyl}phenyl)ethanone. ¹H NMR (400 MHz, DMSO-d₆): δ 8.11-6.55 (m, 9H), 4.56 (m, 1H), 4.44-3.61 (m, 5H), 3.85 (s, 3H). MS (EI) for C₂₂H₁₈F₃N₃O₃, found 430 (MH+).

7-[4-(1H-Imidazol-1-yl)phenyl]-4-{[4-(methylsulfonyl)phenyl]carbonyl}-2,3,4,5-tetrahydro-1,4-benzoxazepine. ¹H NMR (400 MHz, DMSO-d₆): δ 12.0 (s, 1H), 8.42-6.94 (m, 13H), 4.93 (s, 1H), 4.58 (s, 1H), 4.37-4.02 (m, 4H), 3.71 (s, 1H), 3.28 (s, 3H). MS (EI) for C₂₆H₂₃N₃O₄S, found 474 (MH+).

4-{[2-Ethyl-3-fluoro-4-(methylsulfonyl)phenyl]carbonyl}-7-[5-(1H-imidazol-2-yl)-2-thienyl]-2,3,4,5-tetrahydro-1,4-benzoxazepine. ¹H NMR (400 MHz, DMSO-d₆): δ 7.81-6.78 (m, 9H), 4.95 (m, 1H), 4.42 (s, 1H), 4.32-3.97 (m, 3H), 3.61 (s, 1H), 3.32 (s, 3H), 2.65-2.14 (m, 2H), 1.12 and 1.02 (t, 3H). MS (EI) for C₂₆H₂₄FN₃O₄S₂, found 526 (MH+).

3-(4-{[4-(Methylsulfonyl)phenyl]carbonyl}-2,3,4,5-tetrahydro-1,4-benzoxazepin-7-yl)aniline. ¹H NMR (400 MHz, DMSO-d₆): δ 8.03-7.89 (m, 2H), 7.72-7.35 (m, 3H), 7.15-6.99 (m, 2H), 6.70-6.52 (m, 3H), 5.12 (d, 2H), 4.84 (s, 1H), 4.48 (s, 1H), 4.25 (s, 1H), 4.12 (s, 1H), 4.06 (s, 1H), 3.69 (s, 3H), 3.26 (s, 1H). MS (EI) for C₂₃H₂₂N₂O₄S, found 423 (MH+).

4-{[7-(3-Aminophenyl)-2,3-dihydro-1,4-benzoxazepin-4(5H)-yl]carbonyl}benzenesulfonamide. ¹H NMR (400 MHz, DMSO-d₆): δ 7.94-7.82 (m, 3H), 7.63-7.34 (m, 3H), 7.12-7.01 (m, 2H), 7.81 (s, 1H), 6.79-6.68 (m, 2H), 6.60-6.51 (m, 2H), 5.14 (d, 2H), 4.84 (m, 1H), 4.49 (m, 1H), 4.35 (s, 1H), 4.15 (s, 1H), 4.03 (s, 1H), 3.71 (s, 3H). MS (EI) for C₂₂H₂ᵢN₃O₄S, found 424 (MH+).

N-[4-(4-{[4-(Methylsulfonyl)phenyl]carbonyl}-2,3,4,5-tetrahydro-1,4-benzoxazepin-7-yl)phenyl]acetamide. ¹H NMR (400 MHz, DMSO-d₆): δ 10.02 (s, 1H), 8.0 (m, 2H), 7.66-6.73 (m, 9H), 4.85 (s, 1H), 4.47 (s, 1H), 4.26 (s, 1H), 4.16 (s, 1H), 4.05 (s, 1H), 3.68 (s, 1H), 3.28 (s, 3H), 2.03 (s, 3H). MS (EI) for C₂₅H₂₄N₂O₅S, found 465 (MH+).

4-(4-{[4-(Methylsulfonyl)phenyl]carbonyl}-2,3,4,5-tetrahydro-1,4-benzoxazepin-7-yl)aniline. ¹H NMR (400 MHz, DMSO-d₆): δ 8.03 (m, 2H), 7.67-6.55 (m, 9H), 5.2 (s, 2H), 4.8 (s, 1H), 4.45 (s, 1H), 4.23-4.0 (m, 3H), 3.71 (s, 1H), 3.67 (s, 3H). MS (EI) for C₂₃H₂₂N₂O₄S, found 423 (MH+).

N-[3-(4-{[4-(Methylsulfonyl)phenyl]carbonyl}-2,3,4,5-tetrahydro-1,4-benzoxazepin-7-yl)phenyl]acetamide. ¹H NMR (400 MHz, DMSO-d₆): δ 10.02 (d, 1H), 8.19-6.81 (m, 11H), 4.83 (s, 1H), 4.49 (s, 1H), 4.31 (s, 1H), 4.17 (s, 1H), 4.03 (s, 1H), 3.72 (s, 1H), 3.37 (s, 3H), 2.08 (s, 3H). MS (EI) for C₂₅H₂₄N₂O₅S, found 465 (MH+).

N-[3-(4-{[4-(Trifluoroacetyl)phenyl]carbonyl}-2,3,4,5-tetrahydro-1,4-benzoxazepin-7-yl)phenyl]acetamide. ¹H NMR (400 MHz, DMSO-d₆): δ 10.03 (s, 1H), 7.87-6.82 (m, 11H), 4.82 (s, 1H), 4.52 (s, 1H), 4.28 (s, 1H), 4.18 (s, 1H), 4.02 (s, 1H), 3.74 (s, 1H), 2.02 (s, 3H). MS (EI) for C₂₆H₂ᵢF₃N₂O₄, found 483 (MH+).

N-[3-(4-{[4-(Methylsulfonyl)phenyl]carbonyl}-2,3,4,5-tetrahydro-1,4-benzoxazepin-7-yl)phenyl]methanesulfonamide. ¹H NMR (400 MHz, d₆-DMSO): δ 9.84 (s, 1H), 8.01 (m, 2H), 7.69-6.83 (m, 9H), 4.89 (s, 1H), 4.51-4.06 (m, 4H), 3.64 (s, 1H), 3.27 (s, 3H), 3.02 (s, 3H). MS (EI) for C₂₄H₂₄N₂O₆S₂, found 501 (MH+).

2-Amino-5-(4-{[4-(methylsulfonyl)phenyl]carbonyl}-2,3,4,5-tetrahydro-1,4-benzoxazepin-7-yl)phenol. ¹H NMR (400 MHz, d₆-DMSO): δ 9.11 (d, 1H), 8.01 (d, 1H), 7.98 (d, 2H), 7.64 (d, 2H), 7.53-6.58 (m, 4H), 4.81 (s, 1H), 4.68 (s, 2H), 4.47 (s, 1H), 4.23 (s, 1H), 4.13 (s, 3H), 4.02 (s, 1H), 3.68 (s, 3H), 3.27 (s, 3H). MS (EI) for C₂₃H₂₂N₂O₅S, found 439 (MH+).

1-(4-{[7-(4-Amino-3-hydroxyphenyl)-2,3-dihydro-1,4-benzoxazepin-4(5H)-yl]carbonyl}phenyl)-2,2,2-trifluoroethane-1,1-diol. ¹H NMR (400 MHz, DMSO-d₆): δ 9.18 (br.s, 1H), 8.01 (m, 2H), 7.64 (d, 1H), 7.56-7.24 (m, 2H), 7.03-6.58 (m, 5H), 4.81 (s, 1H), 4.65 (br. s, 2H), 4.43 (s, 1H), 4.22 (s, 1H), 4.17 (s, 1H), 4.06 (s, 1H), 3.72 (s, 1H). MS (EI) for C₂₄H₂ᵢF₃N₂O₅, found 475 (MH+).

2,2,2-Trifluoro-1-(4-{[7-(1-methyl-1H-pyrazol-4-yl)-2,3-dihydro-1,4-benzoxazepin-4(5H)-yl]carbonyl}phenyl)ethanol. ¹H NMR (400 MHz, DMSO-d₆): δ 8.14-6.61 (m, 9H), 5.25 (s, 1H), 4.57 (m, 2H), 4.43-3.9 (m, 4H), 3.85 (s, 3H), 3.72 (s, 1H). MS (EI) for C₂₂H₂₀F₃N₃O₃, found 432 (MH+).

4-{[2-Chloro-4-(methylsulfonyl)phenyl]carbonyl}-7-quinolin-3-yl-2,3,4,5-tetrahydro-1,4-benzoxazepine. ¹H NMR (400 MHz, DMSO-d₆): δ 9.26 (d, 0.5H), 9.04 (d, 0.5H), 8.65 (d, 0.5H), 8.29 (d, 0.5H), 8.13-7.90 (m, 4.5H), 7.80-7.71 (m, 2H), 7.68-7.59 (m, 1.5H), 7.43 (d, 0.5H), 7.17-7.13 (m, 1H), 7.04 (d, 0.5H), 5.05-4.86 (m, 1H), 4.60-3.94 (m, 4H), 3.66-3.52 (m, 1H). MS (EI) for C₂₆H₂₁ClN₂O₄S: 482 (M+H).

N-(2-Hydroxyethyl)-4-[(7-quinolin-3-yl-2,3-dihydro-1,4-benzoxazepin-4(5H)-yl)carbonyl]benzenesulfonamide. ¹H NMR (400 MHz, DMSO-d₆): δ 9.31-9.07 (m, 1H), 8.69-8.36 (m, 1H), 8.11-7.41 (m, 10H), 7.22-7.10 (m, 1H), 5.01-3.69 (m, 8H), 2.85-2.77 (m, 2H). MS (EI) for C₂₇H₂₅N₃O₅S: 504 (M+H).

N-Methyl-4-[(7-quinolin-3-yl-2,3-dihydro-1,4-benzoxazepin-4(5H)-yl)carbonyl]benzenesulfonamide. ¹H NMR (400 MHz, DMSO-d₆): δ 9.31-9.08 (m, 1H), 8.70-8.37 (m, 1H), 8.01-7.91 (m, 2H), 7.87-7.74 (m, 2H), 7.69-7.56 (m, 3H), 7.48 (d, 0.5H), 7.22-7.08 (m, 1H), 4.99-4.54 (m, 2H), 4.38-4.20 (m, 2H), 4.10-3.72 (m, 2H), 2.48-2.33 (m, 3H). MS (EI) for C₂₆H₂₃N₃O₄S: 474 (M+H).

N-Propyl-4-[(7-quinolin-3-yl-2,3-dihydro-1,4-benzoxazepin-4(5H)-yl)carbonyl]benzenesulfonamide. ¹H NMR (400 MHz, DMSO-d₆): δ 9.30-9.07 (m, 1H), 8.70-8.36 (m, 1H), 8.10-7.59 (m, 9H), 7.50-7.43 (m, 1H), 7.22-7.04 (m, 1H), 5.00-4.52 (m, 2H), 4.39-4.18 (m, 2H), 4.10-3.70 (m, 2H), 2.75-2.61 (m, 2H), 1.45-1.21 (m, 2H), 0.89-0.61 (m, 3H). MS (EI) for C₂₈H₂₇N₃O₄S: 502 (M+H).

N-Ethyl-4-[(7-quinolin-3-yl-2,3-dihydro-1,4-benzoxazepin-4(5H)-yl)carbonyl]benzenesulfonamide. ¹H NMR (400 MHz, DMSO-d₆): δ 9.30-9.08 (m, 1H), 8.70-8.37 (m, 1H), 8.11-7.60 (m, 9H), 7.46 (d, 0.5H), 7.22-7.06 (m, 1.5H), 4.99-4.56 (m, 2H), 4.39-4.18 (m, 2H), 4.10-3.70 (m, 2H), 2.84-2.67 (m, 2H), 1.02-0.81 (m, 3H). MS (EI) for C₂₇H₂₅N₃O₄S: 488 (M+H).

N-(1-Methylethyl)-4-[(7-quinolin-3-yl-2,3-dihydro-1,4-benzoxazepin-4(5H)-yl)carbonyl]benzenesulfonamide. ¹H NMR (400 MHz, DMSO-d₆): δ 9.30-9.06 (m, 1H), 8.69-8.35 (m, 1H), 8.11-8.01 (m, 2H), 7.97-7.58 (m, 7.5H), 7.46 (d, 1H), 7.22-7.13 (m, 1H), 7.00 (s, 0.5H), 4.99-4.52 (m, 2H), 4.39-4.20 (m, 2H), 4.10-3.69 (m, 2H), 3.33-3.14 (m, 1H), 1.02-0.74 (m, 6H). MS (EI) for C₂₈H₂₇N₃O₄S: 502 (M+H).

4-[(7-Quinolin-3-yl-2,3-dihydro-1,4-benzoxazepin-4(5H)-yl)carbonyl]-N-(tetrahydrofuran-2-ylmethyl)benzenesulfonamide. ¹H NMR (400 MHz, DMSO-d₆): δ 9.30-9.07 (m, 1H), 8.69-8.35 (m, 1H), 8.11-7.58 (m, 9H), 7.46 (d, 0.5H), 7.22-7.07 (m, 1.5H), 4.98-4.53 (m, 2H), 4.39-3.44 (m, 7H), 2.80 (br s, 2H), 1.89-1.37 (m, 4H). MS (EI) for C₃₀H₂₉N₃O₅S: 544 (M+H).

N-Cyclopentyl-4-[(7-quinolin-3-yl-2,3-dihydro-1,4-benzoxazepin-4(5H)-yl)carbonyl]benzenesulfonamide. ¹H NMR (400 MHz, DMSO-d₆): δ 9.30-9.05 (m, 1H), 8.69-8.35 (m, 1H), 8.11-7.41 (m, 9.5H), 7.22-7.11 (m, 1H), 7.02 (s, 0.5H), 4.99-4.52 (m, 2H), 4.39-4.19 (m, 2H), 4.10-3.69 (m, 2H), 3.46-3.31 (m, 1H), 1.65-1.07 (m, 8H). MS (EI) for C₃₀H₂₉N₃O₄S: 528 (M+H).

4-{[4-(Methylsulfonyl)naphthalen-1-yl]carbonyl}-7-quinolin-3-yl-2,3,4,5-tetrahydro-1,4-benzoxazepine. ¹H NMR (400 MHz, CD₃OD): δ 9.20 (d, 0.5H), 8.82-8.73 (m, 1H), 8.62 (d, 1H), 8.44-8.32 (m, 1H), 8.08-7.93 (m, 2.5H), 7.84-7.53 (m, 7H), 7.38-7.32 (m, 0.5H), 7.23-7.14 (m, 1H), 6.20 (d, 0.5H), 5.18-5.08 (m, 1H), 4.65-4.29 (m, 3.5H), 4.12-4.01 (m, 1.5H), 3.29-3.27 (m, 3H). MS (EI) for C₃₀H₂₄N₂O₄S: 509 (M+H).

N-(2-Aminoethyl)-4-[(7-quinolin-3-yl-2,3-dihydro-1,4-benzoxazepin-4(5H)-yl)carbonyl]benzenesulfonamide. ¹H NMR (400 MHz, DMSO-d₆): δ 9.59-9.41 (m, 2H), 9.28-8.98 (m, 1H), 8.36-7.83 (m, 11H), 7.66-7.18 (m, 3H), 4.98-4.64 (m, 2H), 4.40-4.27 (m, 2H), 4.07-3.77 (m, 2H), 3.05-2.97 (m, 2H), 2.90-2.81 (m, 2H). MS (EI) for C₃₀H₂₉N₃O₄S: 503 (M+H).

N-[2-(Dimethylamino)ethyl]-4-[(7-quinolin-3-yl-2,3-dihydro-1,4-benzoxazepin-4(5H)-yl)carbonyl]benzenesulfonamide. ¹H NMR (400 MHz, CD₃OD): δ 9.59-8.94 (m, 2H), 8.39-7.78 (m, 8H), 7.67-7.47 (m, 2H), 70.29-7.07 (m, 1H), 5.04-3.82 (m, 6H), 3.31-3.16 (m, 4H), 2.96-2.88 (m, 6H). MS (EI) for C₂₉H₃₀N₄O₄S: 531 (M+H).

7-Quinolin-3-yl-4-{[6-(trifluoromethyl)pyridin-3-yl]carbonyl}-2,3,4,5-tetrahydro-1,4-benzoxazepine. ¹H NMR (400 MHz, CD₃OD): δ 9.15 (d, 0.5H), 9.01 (d, 0.5H), 8.77 (s, 0.5H), 8.62-8.54 (m, 1H), 8.39 (s, 0.5H), 8.11-7.61 (m, 7.5H), 7.24-7.15 (m, 1H), 7.06 (d, 0.5H), 4.98 (s, 1H), 4.61 (s, 1H), 4.38-4.33 (m, 1H), 4.21-4.16 (m, 2H), 3.91-3.85 (m, 1H). MS (EI) for C₂₅H₁₈F₃N₃O₂: 450 (M+H).

{4-[(7-Quinolin-3-yl-2,3-dihydro-1,4-benzoxazepin-4(5H)-yl)carbonyl]phenyl}methanol. ¹H NMR (400 MHz, d₆-DMSO): δ 9.32-9.08 (m, 1H), 8.70-8.40 (m, 1H), 8.11-7.90 (m, 3H), 7.80-7.60 (m, 3H), 7.44-7.70 (m, 5H), 5.40-5.24 (m, 1H), 4.97-4.82 (m, 1H), 4.67-4.48 (m, 3H), 4.40-4.16 (m, 2H), 4.08-3.75 (m, 2H); MS (EI) for C₂₆H₂₂N₂O₃: 411 (MH⁺).

[4-(Phenylsulfonyl)phenyl](7-quinolin-3-yl-2,3,4,5-tetrahydro-1,5-benzoxazepin-4-yl)methanone. ¹H NMR (400 MHz, d₆-DMSO): δ 9.31-9.04 (m, 1H), 8.70-8.36 (m, 1H), 8.13-7.02 (m, 16H), 4.97-4.50 (m, 2H), 4.37-4.12 (m, 2H), 4.07-3.63 (m, 2H); MS (EI) for C₃₁H₂₄N₂O₄S: 521 (MH⁺).

4-[(3-Ethynylphenyl)carbonyl]-7-quinolin-3-yl-2,3,4,5-tetrahydro-1,4-benzoxazepine. ¹H NMR (400 MHz, d₆-DMSO): δ 9.30-9.06 (m, 1H), 8.69-8.39 (m, 1H), 8.10-7.07 (m, 11H), 4.89 (s, 1H), 4.57 (s, 1H), 4.39-3.94 (m, 4H), 3.75 (br s, 1H); MS (EI) for C₂₇H₂₀N₂O₂: 405 (MH⁺).

7-Quinolin-3-yl-4-({4-[(trifluoromethyl)thio]phenyl}carbonyl)-2,3,4,5-tetrahydro-1,4-benzoxazepine. ¹H NMR (400 MHz, d₆-DMSO): δ 9.30-9.06 (m, 1H), 8.70-8.39 (m, 1H), 8.11-7.92 (m, 3H), 7.85-7.73 (m, 4H), 7.69-7.62 (m, 1H), 7.61-7.54 (m, 1H), 7.44-7.37 (m, 1H), 7.21-7.03 (m, 1H), 4.93 (s, 1H), 4.56 (s, 1H), 4.37-4.19 (m, 2H), 4.09-4.01 (m, 1H), 3.79-3.72 (m, 1H); MS (EI) for C₂₆H₁₉F₃N₂O₂S: 481 (MH⁺).

2-Methyl-2-{4-[(7-quinolin-3-yl-2,3-dihydro-1,4-benzoxazepin-4(5H)-yl)carbonyl]phenyl}propanenitrile. ¹H NMR (400 MHz, CDCl₃): δ 9.19 (s, 0.5H), 9.04 (s, 0.5H), 8.35 (s, 0.5H), 8.19-8.11 (m, 1.5H), 7.90 (dd, 1H), 7.83 (s, 0.5H), 7.73 (td, 1H), 7.63-7.49 (m, 4H), 7.43 (d, 2H), 7.26-7.17 (m, 1H), 6.95 (s, 0.5H), 4.93 (br s, 1H), 4.59 (br s, 1H), 4.33 (br s, 1H), 4.20 (br s, 1H), 4.11 (br s, 1H), 3.88 (br s, 1H), 1.74 (s, 6H). MS (EI) for C₂₉H₂₅N₃O₂: 448.1 (MH⁺).

4-({4-[(4-Methylpiperazin-1-yl)sulfonyl]phenyl}carbonyl)-7-quinolin-3-yl-2,3,4,5-tetrahydro-1,4-benzoxazepine. ¹H NMR (400 MHz, MeOD-d₄): δ 9.17, 9.00 (s, 1H), 8.60 (s, 1H), 8.35 (s, 1H), 8.04 (m, 2H), 7.88 (m, 2H), 7.78 (m, 1H), 7.73-7.63 (m, 3H), 7.51 (m, 1H), 7.20 (m, 1H), 6.97 (s, 1H), 4.96 (s, 1H), 4.57 (s, 1H), 4.35 (m, 1H), 4.17 (m, 2H), 3.83 (m, 1H), 5.04 (m, 4H), 2.51 (m, 2H), 2.40 (m, 2H), 2.27-2.18 (d, 3H); MS (EI) for C₃₀H₃₀N₄O₄S: 543.2 (MH⁺).

N-Cyclopropyl-4-[(7-quinolin-3-yl-2,3-dihydro-1,4-benzoxazepin-4(5H)-yl)carbonyl]benzamide. ¹H NMR (400 MHz, MeOD-d₄): δ 9.29-8.98 (m, 1H), 8.62-8.31 (m, 1H), 8.09-8.00 (m, 2H), 7.95-7.84 (m, 2.5H), 7.81-7.63 (m, 3H), 7.50-7.37 (m, 2H), 7.23-7.17 (m, 1H), 6.97-6.94 (m, 0.5H), 4.96 (s, 1H), 4.64 (s, 1H), 4.38-4.30 (m, 1H), 4.22-4.11 (m, 2H), 3.91-3.83 (m, 1H), 2.90-2.80 (m, 1H), 0.85-0.77 (m, 2H), 0.67-0.58 (m, 2H). MS (EI) for C₂₉H₂₅N₃O₃: 464.2 (MH⁺).

N-(Phenylmethyl)-4-[(7-quinolin-3-yl-2,3-dihydro-1,4-benzoxazepin-4(5H)-yl)carbonyl]benzamide. ¹H NMR (400 MHz, MeOD-d₄): δ 9.20-8.99 (m, 1H), 8.62-8.33 (m, 1H), 8.09-7.86 (m, 5H), 7.81-7.60 (m, 3H), 7.53-7.40 (dd, 2H), 7.38-7.17 (m, 5.5H), 7.02-6.98 (m, 0.5H), 4.96 (s, 1H), 4.66 (s, 1H), 4.58 (s, 2H), 4.38-4.31 (m, 1H), 4.21-4.12 (m, 2H), 3.90-3.84 (m, 1H). MS (EI) for C₃₃H₂₇N₃O₃: 514.2 (MH⁺).

N-Phenyl-4-[(7-quinolin-3-yl-2,3-dihydro-1,4-benzoxazepin-4(5H)-yl)carbonyl]benzamide. ¹H NMR (400 MHz, MeOD-d₄): δ 9.20-8.99 (m, 1H), 8.63-8.31 (m, 1H), 8.11-7.96 (m, 4H), 7.92-7.63 (m, 5H), 7.58-6.88 (m, 7H), 4.98 (s, 1H), 4.68 (s, 1H), 4.22-4.15 (m, 2H), 3.93-3.87 (m, 1H). MS (EI) for C₃₂H₂₅N₃O₃: 500.2 (MH⁺).

N-Ethyl-4-[(7-quinolin-3-yl-2,3-dihydro-1,4-benzoxazepin-4(5H)-yl)carbonyl]benzamide. ¹H NMR (400 MHz, MeOD-d₄): δ 9.58-8.98 (m, 2H), 8.43-8.04 (m, 3H), 8.03-7.78 (m, 5H), 7.55-7.07 (m, 3H), 4.99 (s, 1H), 4.67 (s, 1H), 4.42-4.35 (m, 1H), 4.23-4.14 (m, 2H), 3.93-3.86 (m, 1H), 3.41 (q, 2H), 1.21 (m, 3H). MS (EI) for C₂₈H₂₅N₃O₃: 452.2 (MH⁺).

4-[(2-Methyl-4-nitrophenyl)carbonyl]-7-quinolin-3-yl-2,3,4,5-tetrahydro-1,4-benzoxazepine. ¹H NMR (400 MHz, CDCl₃): δ 9.22 (br s, 0.4H), 9.05 (br s, 0.6H), 8.36 (br s, 0.4H), 8.24-8.01 (m, 3.5H), 7.94-7.86 (m, 1H), 7.81 (br d, 0.5H), 7.74 (br t, 1H), 7.67-7.57 (m, 2H), 7.36-7.29 (m, 1H), 7.29-7.18 (m, 1H), 6.71 (br d, 0.6H), 5.09 (br d, 0.5H), 4.88 (br d, 0.5H), 4.43 (br q, 1H), 4.34-4.23 (m, 2H), 4.22-3.98 (m, 1H), 3.75-3.55 (m, 1H), 2.38 (s, 1H), 2.19 (s, 2H). MS (EI) for C₂₆H₂ᵢN₃O₄: 440.0 (MH⁺).

2,2,2-Trifluoro-1-{4-[(7-pyridin-3-yl-2,3-dihydro-1,4-benzoxazepin-4(5H)-yl)carbonyl]phenyl}ethanone. ¹H NMR (400 MHz, DMSO-d₆): δ 9.08-7.08 (m, 11H), 4.85-3.9 (m, 6H). MS (EI) for C₂₃H₁₇F₃N₂O₅, found 445 (M+1+H₂O).

2,2,2-Trifluoro-1-(4-{[7-(1H-indazol-6-yl)-2,3-dihydro-1,4-benzoxazepin-4(5H)-yl]carbonyl}phenyl)ethanone. ¹H NMR (400 MHz, DMSO-d₆): δ 13.05 (m, 1H), 8.09-6.80 (m, 11H), 4.90-3.76 (m, 6H). MS (EI) for C₂₅H₁₈F₃N₃O₃, found 484 (M+1+H₂O).

4-(5-Methyl-7-(quinolin-3-yl)-2,3,4,5-tetrahydrobenzo[f][1,4]oxazepine-4-carbonyl)benzenesulfonamide. ¹H NMR (400 MHz, DMSO-d₆): δ 9.28-7.07 (m, 15H), 5.0-3.60 (m, 5H), 1.69-1.55 (m, 3H). MS (EI) for C₂₆H₂₃N₃O₄S, found 474 (MH+).

2,2,2-Trifluoro-1-(4-{[7-(1H-pyrazol-4-yl)-2,3-dihydro-1,4-benzoxazepin-4(5H)-yl]carbonyl}phenyl)ethanone. ¹H NMR (400 MHz, DMSO-d₆): δ 12.90 (s, 1H), 8.38-6.65 (m, 9H), 4.81 (s, 1H), 4.42-3.91 (m, 4H), 3.72 (s, 1H). MS (EI) for C₂₁H₁₆F₃N₃O₃, found 416 (MH+).

2,2,2-Trifluoro-1-[4-({7-[1-(2-methylpropyl)-1H-pyrazol-4-yl]-2,3-dihydro-1,4-benzoxazepin-4(5H)-yl}carbonyl)phenyl]ethanone. ¹H NMR (400 MHz, DMSO-d₆): δ 8.10 (s, 1H), 7.85 (s, 1H), 7.65-6.97 (m, 6H), 6.65 (m, 1H), 4.78 (s, 1H), 4.27 (s, 1H), 4.19-3.83 (m, 5H), 3.72 (s, 1H), 2.03 (m, 1H), 1.19 (d, 6H). MS (EI) for C₂₅H₂₄F₃N₃O₃, found 472 (MH+).

2,2,2-Trifluoro-1-[4-({7-[1-(2-methylpropyl)-1H-pyrazol-4-yl]-2,3-dihydro-1,4-benzoxazepin-4(5H)-yl}carbonyl)phenyl]ethanol. ¹H NMR (400 MHz, DMSO-d₆): δ 8.12 (s, 1H), 7.825 (s, 1H), 7.57 (m, 2H), 7.43-7.25 (m, 3H), 7.03-6.625 (m, 2H), 5.29 (br.s, 1H), 4.78 (s, 1H), 4.41 (s, 1H), 4.32-4.03 (m, 4H), 4.02 (s, 1H), 3.72 (s, 1H), 2.03 (m, 1H), 1.18 (m, 6H). MS (EI) for C₂₅H₂₆F₃N₃O₃, found 468 (MH+).

2,2,2-Trifluoro-1-(4-{[7-(1H-pyrazol-4-yl)-2,3-dihydro-1,4-benzoxazepin-4(5H)-yl]carbonyl}phenyl)ethanol. ¹H NMR (400 MHz, DMSO-d₆): δ 8.74 (s, 1H), 8.48-6.68 (m, 9H), 5.25 (s, 1H), 4.81 (s, 1H), 4.45-3.60 (m, 5H). MS (EI) for C₂₁H₁₈F₃N₃O₃, found 418 (MH+).

N-Cyclobutyl-4-{[7-(quinolin-3-yl)-2,3-dihydro-1,4-benzoxazepin-4(5H)-yl]carbonyl}benzamide. ¹H NMR (400 MHz, Methanol-d₄): δ 9.18 (s, 0.5H), 9.00 (s, 0.5H), 8.60 (s, 0.5H), 8.32 (s, 0.5H), 8.10-7.85 (m, 4.5H), 7.82-7.62 (m, 3H), 7.52-7.37 (m, 2H), 7.25-6.95 (m, 1H), 6.73 (s, 0.5H), 4.96 (m, 1H), 4.65 (m, 1H), 4.55-4.45 (m, 1H), 4.37-4.31 (m, 1H), 4.21-4.13 (m, 2H), 3.90-3.83 (m, 1H), 2.41 (m, 2H), 2.18-2.02 (m, 2H), 1.84-1.71 (m, 2H). MS (EI) for C₃₀H₂₇N₃O₃, found 478 (MH+).

N-(Cyclopropylmethyl)-4-{[7-(quinolin-3-yl)-2,3-dihydro-1,4-benzoxazepin-4(5H)-yl]carbonyl}benzamide. ¹H NMR (400 MHz, Methanol-d₄): δ 8.93 (s, 0.5H), 8.76 (s, 0.5H), 8.35 (s, 0.5H), 8.09 (s, 0.5H), 7.84-7.62 (m, 4.5H), 7.56-7.37 (m, 3H), 7.28-7.14 (m, 2H), 6.99-6.92 (m, 1H), 6.73 (s, 0.5H), 4.71 (s, 1H), 4.41 (s, 1H), 4.12-4.07 (m, 1H), 3.95-3.88 (m, 2H), 3.65-3.60 (m, 1H), 2.99 (d, 2H), 0.90-0.77 (m, 1H), 0.31-0.20 (m, 2H), 0.07-0.03 (2H). MS (EI) for C₃₀H₂₇N₃O₃, found 478 (MH+).

[5-(4-Fluoro-2-methylphenyl)-1-methyl-1H-pyrrol-2-yl][7-(2-methyl-1H-benzimidazol-6-yl)-2,3-dihydro-1,4-benzoxazepin-4(5H)-yl]methanone. ¹H NMR (400 MHz, DMSO-d₆): δ 12.10 (br, 1H), 7.51 (m, 7H), 7.01 (d, 1H), 6.45 (s, 1H), 6.122 (s, 1H), 4.83 (s, 3H), 4.30 (brs, 2H), 4.0 (brs, 2H), 3.18 (s, 3H), 2.50 (s, 3H), 2.05 (s, 3H). MS (EI) for C₃₀H₂₇FN₄O₂: 495.5 (MH⁺).

4-{[1-Ethyl-5-(methylsulfonyl)-1H-indol-2-yl]carbonyl}-7-(2-methyl-1H-benzimidazol-6-yl)-2,3,4,5-tetrahydro-1,4-benzoxazepine. ¹H NMR (400 MHz, DMSO-d₆): δ 12.25 (m, 1H), 8.34-8.18 (m, 1H), 7.91-7.32 (m, 6H), 7.18-6.75 (m, 2H), 5.03-4.75 (m, 2H), 4.41-3.94 (m, 6H), 3.26-3.12 (s, 3H), 1.29-0.96 (d, 3H). MS (EI) for C₂₉H₂₈N₄O₄S, found 529 (MH+).

[5-(4-Methoxyphenyl)-1-methyl-1H-pyrrol-2-yl][7-(2-methyl-1H-benzimidazol-6-yl)-2,3-dihydro-1,4-benzoxazepin-4(5H)-yl]methanone. ¹H NMR (400 MHz, DMSO-d₆): δ 12.4 (br, 1H), 7.63 (s, 1H), 7.50 (m, 2H), 7.29 (m, 3H), 7.06 (d, 2H), 6.92 (m, 2H), 6.36 (s, 1H), 6.11 (d, 1H), 4.83 (s, 2H), 4.25 (s, 2H), 4.05 (s, 2H), 3.76 (s, 3H), 3.30 (s, 3H), 2.47 (s, 3H). MS (EI) for C₃₀H₂₈N₄O₃: 493.2 (MH⁺).

4-{[1-Ethyl-5-(methyloxy)-1H-pyrrolo[3,2-b]pyridin-2-yl]carbonyl}-7-(2-methyl-1H-benzimidazol-6-yl)-2,3,4,5-tetrahydro-1,4-benzoxazepine. ¹H NMR (400 MHz, DMSO-d₆): δ 12.32-12.15 (m, 1H), 8.03-7.92 (m, 1H), 7.80-6.47 (m, 8H), 4.97-4.79 (m, 2H), 4.39-3.82 (m, 9H), 1.32-0.97 (m, 3H). MS (EI) for C₂₈H₂₇N₅O₃, found 482 (MH+).

4-{[1-Ethyl-5-(methyloxy)-1H-pyrrolo[2,3-c]pyridin-2-yl]carbonyl}-7-(2-methyl-1H-benzimidazol-6-yl)-2,3,4,5-tetrahydro-1,4-benzoxazepine. ¹H NMR (400 MHz, DMSO-d₆): δ 12.30-12.16 (m, 1H), 8.56 (m, 1H), 7.78-7.28 (m, 4H), 7.13-6.84 (m, 3H), 6.60-6.39 (m, 1H), 4.93 (s, 1H), 4.76 (s, 1H), 4.34-3.81 (m, 9H), 1.33-1.01 (m, 3H). MS (EI) for C₂₈H₂₇N₅O₃, found 482 (MH+).

[7-(2-Methyl-1H-benzimidazol-6-yl)-2,3-dihydro-1,4-benzoxazepin-4(5H)-yl]{1-methyl-5-[4-(propan-2-ylsulfonyl)phenyl]-1H-pyrrol-2-yl}methanone. ¹H NMR (400 MHz, DMSO-d₆): δ 12.10 (br, 1H), 7.57 (m, 10H), 7.02 (d, 1H), 6.48 (s, 1H), 4.81 (s, 2H), 4.35 (s, 2H), 4.02 (br s, 2H), 3.30 (s, 4H), 2.51 (s, 3H), 3.33 (br, 3H), 2.51 (s, 3H). MS (EI) for C₃₂H₃₂N₄O₄S: 569.1 (MH⁺).

(5-Bromo-1-methyl-1H-pyrrol-2-yl)[7-(2-methyl-1H-benzimidazol-6-yl)-2,3-dihydro-1,4-benzoxazepin-4(5H)-yl]methanone. ¹H NMR (400 MHz, DMSO-d₆): δ 12.10 (br, 1H), 7.62 (s, 1H), 7.51 (d, 2H), 7.41 (s, 1H), 7.01 (d, 2H), 6.25 (s, 2H), 4.98 (s, 2H), 4.30 (br s, 2H), 4.0 (br s, 2H), 3.50 (s, 3H), 2.50 (s, 3H). MS (EI) for C₂₃H₂₁BrN₄O₂: 466.9 (MH⁺).

[7-(2-Methyl-1H-benzimidazol-6-yl)-2,3-didhydro-1,4-benzoxazepin-4(5H)-yl](2-phenyl-1,3-thiazol-4-yl)methanone. ¹H NMR (400 MHz, DMSO-d₆): δ 12.3 (br, 1H), 8.21 (s, 1H), 7.54 (m, 11H), 4.10 (s, 1H), 4.82 (s, 1H), 4.27 (d, 2H), 4.12 (d, 2H) 2.49 (s, 3H). MS (EI) for C₂₇H₂₂N₄O₂S: 467 (MH⁺).

[7-(2-Methyl-1H-benzimidazol-6-yl)-2,3-dihydro-1,4-benzoxazepin-4(5H)-yl][5-(pyridin-2-yl)thiophen-2-yl]methanone. ¹H NMR (400 MHz, DMSO-d₆): δ 12.2 (br, 1H), 8.51 (d, 1H), 8.01 (d, 1H), 7.80 (m, 2H), 7.65 (s, 1H), 7.38 (m, 6H), 7.02 (d, 1H), 4.98 (s, 2H), 4.30 (s, 2H), 4.10 (s, 2H), 2.51 (s, 3H). MS (EI) for C₂₇H₂₂N₄O₂S: 467.1 (MH⁺).

[7-(2-Methyl-1H-benzimidazol-6-yl)-2,3-dihydro-1,4-benzoxazepin-4(5H)-yl][4-(1,3-oxazol-5-yl)phenyl]methanone. ¹H NMR (400 MHz, DMSO-d₆): δ 12.10 (br, 1H), 7.57 (m, 10H), 7.01 (d, 2H), 4.81 (s, 2H), 4.35 (s, 2H), 4.02 (br s, 2H), 2.51 (s, 3H). MS (EI) for C₂₇H₂₂N₄O₃: 451.1 (MH⁺).

[7-(2-Methyl-1H-benzimidazol-6-yl)-2,3-dihydro-1,4-benzoxazepin-4(5H)-yl][4-(1,3-thiazol-2-yl)phenyl]methanone. ¹H NMR (400 MHz, DMSO-d₆): δ 12.10 (br, 1H), 8.00 (m, 3H), 7.82 (s, 1H), 7.60 (s, 1H), 7.50 (m, 5H), 7.02 (m, 2H), 4.81 (s, 1H), 4.65 (s, 1H), 4.25 (m, 3H), 3.78 (s, 1H), 2.51 (s, 3H). MS (EI) for C₂₇H₂₂N₄O₂S: 467.2 (MH⁺).

4-[(1,5-Dimethyl-1H-pyrrol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.26 (s, 1H), 7.73-7.29 (m, 5H), 7.08-7.00 (m, 1H), 6.18 (m, 1H), 5.87-5.81 (m, 1H), 4.80 (s, 2H), 4.23 (m, 2H), 4.01 (s, 2H), 3.42 (s, 3H), 2.17 (s, 3H). MS (EI) for C₂₄H₂₄N₄O₂, found 401 (MH+).

4-[(1-Ethyl-5-methyl-1H-pyrrol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.24 (m, 1H), 7.68-7.29 (m, 5H), 7.03 (m, 1H), 6.28 (m, 1H), 5.84 (m, 1H), 4.82 (s, 2H), 4.27 (m, 2H), 4.00 (m, 2H), 3.91 (m, 2H), 2.20 (s, 2H), 1.05-0.94 (m, 3H). MS (EI) for C₂₅H₂₆N₄O₂, found 415 (MH+).

7-(2-Methyl-1H-benzimidazol-6-yl)-4-[(1-methyl-1H-pyrrolo[2,3-b]pyridin-2-yl)carbonyl]-2,3,4,5-tetrahydro-1,4-benzoxazepine. ¹H NMR (400 MHz, DMSO-d₆): δ 12.24 (s, 1H), 8.38 (s, 1H), 8.04 (m, 1H), 7.88-6.47 (m, 8H), 4.97-4.74 (m, 2H), 4.39-4.16 (m, 2H), 4.13-4.00 (s, 2H), 3.81-3.52 (m, 3H). MS (EI) for C₂₆H₂₃N₅O₂, found 438 (MH+).

4-[(1-Ethyl-1H-pyrrolo[2,3-b]pyridin-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.24 (s, 1H), 8.36 (s, 1H), 8.10-8.01 (m, 1H), 7.86-6.89 (m, 7H), 6.78-6.53 (m, 1H), 5.00-4.78 (m, 2H), 4.41-3.95 (m, 6H), 1.32-1.00 (m, 3H). MS (EI) for C₂₇H₂₅N₅O₂, found 452 (MH+).

4-{[3-Fluoro-2-methyl-4-(methylsulfonyl)phenyl]carbonyl}-7-quinolin-3-yl-2,3,4,5-tetrahydro-1,4-benzoxazepine. ¹H NMR (400 MHz, DMSO-d₆): δ 9.29-9.07 (m, 1H), 8.68-8.34 (m, 1H), 8.09-7.64 (m, 6H), 7.32-7.06 (m, 2H), 4.99 (m, 1H), 4.52-4.08 (m, 4H), 3.60 (m, 1H), 3.34 (m, 3H), 2.14-1.85 (m, 3H). MS (EI) for C₂₇H₂₃FN₂O₄S, found 491 (MH+).

4-{[2-Bromo-4-(methylsulfonyl)phenyl]carbonyl}-7-quinolin-3-yl-2,3,4,5-tetrahydro-1,4-benzoxazepine. ¹H NMR (400 MHz, DMSO-d₆): δ 9.29-9.07 (m, 1H), 8.68-8.31 (m, 1H), 8.27-8.21 (m, 1H), 8.09-7.96 (m, 3H), 7.81-7.65 (m, 3H), 7.43-7.04 (m, 2H), 5.07-4.89 (m, 1H), 4.59-3.98 (m, 4H), 3.59 (m, 1H), 3.34-3.17 (m, 3H). MS (EI) for C₂₆H₂₁BrN₂O₄S, found 538 (MH+).

4-{[2-Ethyl-4-(methylsulfonyl)phenyl]carbonyl}-7-quinolin-3-yl-2,3,4,5-tetrahydro-1,4-benzoxazepine. ¹H NMR (400 MHz, DMSO-d₆): δ 9.29-9.05 (m, 1H), 8.68-8.31 (m, 1H), 8.08-7.62 (m, 7H), 7.47-7.29 (m, 1H), 7.19-6.90 (m, 2H), 5.10-4.89 (m, 1H), 4.53-4.12 (m, 4H), 3.58 (br.m, 1H), 3.27 (s, 3H), 2.60 (m, 1H), 2.34 (m, 1H), 1.15-1.03 (m, 3H). MS (EI) for C₂₈H₂₆N₂O₄S, found 487 (MH+).

(5-{[7-(2-Methyl-1H-benzimidazol-6-yl)-2,3-dihydro-1,4-benzoxazepin-4(5H)-yl]carbonyl}-4H-thieno[3,2-b]pyrrol-4-yl)acetonitrile. ¹H NMR (400 MHz, DMSO-d₆): δ 12.24 (s, 1H), 7.77-7.30 (m, 7H), 7.10-6.81 (m, 2H), 5.44 (s, 2H), 4.91 (s, 2H), 4.31 (s, 2H), 4.13 (s, 2H). MS (EI) for C₂₆H₂₁N₅O₂S, found 468 (MH+).

N,N-Dimethyl-2-(2-methyl-5-{[7-(2-methyl-1H-benzimidazol-6-yl)-2,3-dihydro-1,4-benzoxazepin-4(5H)-yl]carbonyl}-4H-thieno[3,2-b]pyrrol-4-yl)acetamide. ¹H NMR (400 MHz, DMSO-d₆); M2.24 (s, 1H), 7.75-7.32 (m, 5H), 7.10-7.02 (m, 1H), 6.86 (s, 1H), 6.53 (s, 1H), 5.09 (s, 2H), 4.77 (s, 2H), 4.22 (s, 2H), 4.13-3.98 (s, 1H), 2.90 (s, 3H), 2.69-2.54 (m, 3H), 2.47 (s, 3H). MS (EI) for C₂₉H₂₉N₅O₃S, found 528 (MH+).

4-{[2-Ethyl-4-(ethylsulfonyl)-3-fluorophenyl]carbonyl}-9-fluoro-7-(2-methyl-1H-benzimidazol-6-yl)-2,3,4,5-tetrahydro-1,4-benzoxazepine. ¹H NMR (400 MHz, DMSO-d₆): δ 7.76-6.59 (m, 7H), 4.91 (m, 1H), 4.47-4.03 (m, 4H), 3.63 (s, 1H), 3.4 (m, 2H), 2.67-2.16 (m, 2H), 1.21 (m, 4H), 1.02 (m, 6H). MS (EI) for C₂₈H₂₇F₂N₃O₄S, found 540 (MH+).

4-{[2-Ethyl-3-fluoro-4-(methylsulfonyl)phenyl]carbonyl}-9-fluoro-7-(2-methyl-1H-benzimidazol-6-yl)-2,3,4,5-tetrahydro-1,4-benzoxazepine. ¹H NMR (400 MHz, DMSO-d₆): δ 7.74-6.62 (m, 7H), 4.96 (m, 1H), 4.49-4.03 (m, 4H), 3.62 (m, 1H), 3.61 (s, 1H), 2.38 (s, 3H), 3.30 (s, 3H), 1.12 (m, 2H), 1.01 (m, 3H). MS (EI) for C₂₇H₂₅F₂N₃O₄S, found 526 (MH+).

4-[(2-Chloro-4-ethyl-4H-thieno[3,2-b]pyrrol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.23 (s, 1H), 7.71-7.19 (m, 6H), 7.09-6.60 (m, 2H), 4.86 (s, 2H), 4.28-4.20 (m, 2H), 4.15-4.00 (m, 4H), 1.16-0.99 (m, 3H). MS (EI) for C₂₆H₂₃ClN₄O₂S, found 491, 493 (M, M+2).

4-{[2-Ethyl-3-fluoro-4-(methylsulfonyl)phenyl]carbonyl}-8-fluoro-7-(2-methyl-1H-benzimidazol-6-yl)-2,3,4,5-tetrahydro-1,4-benzoxazepine. ¹H NMR (400 MHz, DMSO-d₆): δ 12.3 (s, 1H), 8.76-6.68 (m, 7H), 4.9 (m, 1H), 4.54-4.01 (m, 4H), 3.52 (s, 1H), 3.31 (s, 3H), 2.64-2.36 (m, 2H), 2.73-2.15 (m, 2H), 1.03 (m, 3H). MS (EI) for C₂₇H₂₅F₂N₃O₄S, found 526 (MH+).

4-{[2-ethyl-4-(1,3-thiazol-2-yl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 12.22 (s, 1H), 8.02-7.76 (m, 4H), 7.68 (m, 1H), 7.57-6.60 (m, 6H), 5.07-4.77 (m, 1H), 4.53-4.04 (m, 4H), 3.67-3.56 (m, 1H), 2.68-2.24 (m, 2H), 1.27-0.98 (m, 3H); MS (EI) for C₂₉H₂₆N₄O₂S: 495.2 (MH+).

7-(2-Methyl-1H-benzimidazol-6-yl)-4-{[2-methyl-4-(1,3-thiazol-2-yl)phenyl]carbonyl}-2,3,4,5-tetrahydro-1,4-benzoxazepine. ¹H NMR (400 MHz, DMSO-d₆): δ 12.31-12.09 (m, 1H), 8.04-7.17 (m, 9H), 7.10-6.57 (m, 2H), 5.04-4.75 (m, 1H), 4.44 (s, 1H), 4.36-3.97 (m, 3H), 3.60 (s, 1H), 2.47 (s, 3H), 2.24-1.90 (m, 3H); MS (EI) for C₂₈H₂₄N₄O₂S: 481.2 (MH+).

4-{[7-(6-Aminopyridin-3-yl)-2,3-dihydro-1,4-benzoxazepin-4(5H)-yl]carbonyl}-N-(1,1-dimethylethyl)-3-methylbenzenesulfonamide. ¹H NMR (400 MHz, Methanol-d₄): δ 8.17-7.72 (m, 3H), 7.58-7.25 (m, 3H), 7.10-7.05 (m, 1H), 6.43 (m, 1H), 5.05-4.80 (m, 1H), 4.50-4.04 (m, 4H), 3.75-3.55 (m, 1H), 3.35 (m, 3H), 1.16 s, 9H). MS (EI) for C₂₆H₃₀N₄O₄S, found 495 (MH+).

4-{[7-(6-Aminopyridin-3-yl)-2,3-dihydro-1,4-benzoxazepin-4(5H)-yl]carbonyl}-3-methyl-N-(1-methylethyl)benzenesulfonamide. ¹H NMR (400 MHz, Methanol-d₄): δ 8.20-7.70 (m, 3H), 7.58-7.28 (m, 3H), 7.11-7.04 (m, 1H), 6.69-6.61 (m, 1H), 4.98-4.81 (m, 1H), 4.48-4.02 (m, 4H), 3.70-3.60 (m, 1H), 3.43-3.35 (m, 1H), 3.34 (s, 3H), 1.01 (m, 6H). MS (EI) for C₂₅H₂₈N₄O₄S, found 481 (MH+).

[7-(6-Aminopyridin-3-yl)-2-methyl-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.24-8.03 (m, 1H), 7.81-7.72 (m, 1H), 7.56-7.39 (m, 2H), 7.27-6.99 (m, 3H), 6.53-6.45 (dd, 1H), 6.05 (s, 2H), 5.09-4.31 (m, 2H), 4.37-3.50 (m, 3H), 3.39-3.34 (m, 3H), 2.69-1.79 (m, 2H), 1.38-1.37 (d, 3H), 1.17-0.90 (m, 3H). MS (EI) for C₂₅H₂₆FN₃O₄S, found 471 (MH+).

4-(4-{[3-Fluoro-2-methyl-4-(methylsulfonyl)phenyl]carbonyl}-2,3,4,5-tetrahydro-1,4-benzoxazepin-7-yl)-N-methylbenzamide. ¹H NMR (400 MHz, DMSO-d₆): δ 8.50-8.42 (m, 1H), 7.97-7.69 (m, 4H), 7.65-7.47 (m, 2H), 7.33-6.80 (m, 2H), 5.01-4.89 (m, 1H), 4.57-3.92 (m, 5H), 3.67-3.48 (m, 1H), 2.16-1.73 (m, 3H). MS (EI) for C₂₆H₂₅FN₂O₅S, found 497 (MH+).

4-(4-{[3-Fluoro-2-methyl-4-(methylsulfonyl)phenyl]carbonyl}-2,3,4,5-tetrahydro-1,4-benzoxazepin-7-yl)-N-(1-methylethyl)benzamide. ¹H NMR (400 MHz, DMSO-d₆): δ 8.35-8.23 (m, 1H), 7.98-7.69 (m, 4H), 7.64-7.47 (m, 2H), 7.33-6.80 (m, 2H), 5.02-4.85 (m, 1H), 4.57-3.92 (m, 5H), 3.67-3.48 (m, 1H), 2.16-1.75 (m, 3H), 1.22-1.11 (m, 6H). MS (EI) for C₂₈H₂₉FN₂O₅S, found 525 (MH+).

[(2S)-7-(6-Aminopyridin-3-yl)-2-methyl-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.24-8.03 (m, 1H), 7.81-7.72 (m, 1H), 7.56-7.39 (m, 2H), 7.27-6.99 (m, 3H), 6.53-6.45 (dd, 1H), 6.05 (s, 2H), 5.09-4.31 (m, 2H), 4.37-3.50 (m, 3H), 3.39-3.34 (dd, 3H), 2.69-1.79 (m, 2H), 1.38-1.37 (d, 3H), 1.17-0.90 (m, 3H). MS (EI) for C₂₅H₂₆FN₃O₄S, found 471 (MH+).

5-(4-{[2-Ethyl-3-fluoro-4-(methylsulfonyl)phenyl]carbonyl}-2,3,4,5-tetrahydro-1,4-benzoxazepin-7-yl)-N-methylthiophene-2-carboxamide. ¹H NMR (400 MHz, DMSO-d₆): δ 8.51-8.42 (m, 1H), 7.73-7.67 (m, 2H), 7.58-7.53 (m, 2H), 7.31-7.23 (m, 1H), 7.08-7.02 (m, 1H), 6.97 (m, 1H), 5.03-4.78 (m, 1H), 4.84-3.95 (m, 4H), 3.62-3.55 (m, 1H), 3.41 (s, 3H), 2.80-2.74 (m, 3H), 2.48-2.28 (m, 2H), 1.04 (m, 3H). MS (EI) for C₂₅H₂₅FN₂O₅S₂, found 517 (MH+).

4-(4-{[2-Ethyl-3-fluoro-4-(methylsulfonyl)phenyl]carbonyl}-2,3,4,5-tetrahydro-1,4-benzoxazepin-7-yl)-N-methylbenzamide. ¹H NMR (400 MHz, DMSO-d₆): δ 8.55-8.45 (m, 1H), 7.99-7.70 (m, 4H), 7.63-7.48 (m, 2H), 7.32-6.82 (m, 2H), 5.08-4.81 (m, 1H), 4.53-3.99 (m, 4H), 3.67-3.53 (m, 1H), 3.38-3.35 (m, 3H), 2.80 (m, 3H), 2.70-1.97 (m, 2H), 1.14-0.95 (m, 3H). MS (EI) for C₂₇H₂₇FN₂O₅S, found 511 (MH+).

4-(4-{[2-Ethyl-3-fluoro-4-(methylsulfonyl)phenyl]carbonyl}-2,3,4,5-tetrahydro-1,4-benzoxazepin-7-yl)-2-fluoro-N-methylbenzamide. ¹H NMR (400 MHz, Methanol-d₄): 7.88-7.72 (m, 2H), 7.61-7.49 (m, 3H), 7.35-7.10 (m, 3H), 5.05-4.85 (m, 1H), 4.60-3.96 (m, 4H), 3.65-3.50 (m, 1H), 3.40-3.26 (m, 2H), 2.16-1.76 (dd, 3H), 1.14 (dt, 3H). MS (EI) for C₂₇H₂₆F₂N₂O₅S, found 529 (MH+).

N-Ethyl-4-(4-{[2-ethyl-3-fluoro-4-(methylsulfonyl)phenyl]-carbonyl}-2,3,4,5-tetrahydro-1,4-benzoxazepin-7-yl)benzamide. ¹H NMR (400 MHz, DMSO-d₆): δ 8.57-8.44 (m, 1H), 7.97-7.67 (m, 4H), 7.64-7.44 (m, 2H), 7.29-6.80 (m, 2H), 5.08-4.77 (m, 1H), 4.52-3.97 (m, 4H), 3.63-3.51 (m, 1H), 3.36-3.33 (m, 3H), 3.31-3.23 (m, 2H), 2.69-1.92 (m, 2H), 1.17-0.91 (m, 6H). MS (EI) for C₂₈H₂₉FN₂O₅S, found 525 (MH+).

4-(4-{[2-Ethyl-4-(ethylsulfonyl)-3-fluorophenyl]carbonyl}-2,3,4,5-tetrahydro-1,4-benzoxazepin-7-yl)-N-methylbenzamide. ¹H NMR (400 MHz, DMSO-d₆); δ 8.54-8.44 (m, 1H), 7.98-7.69 (m, 4H), 7.66-7.46 (m, 2H), 7.32-6.78 (m, 2H), 5.08-4.80 (m, 1H), 4.45 (m, 1H), 4.34-4.04 (m, 3H), 3.67-3.53 (m, 1H), 3.47-3.35 (m, 2H), 2.80 (m, 3H), 2.70-1.96 (m, 2H) 1.21-0.94 (m, 6H). MS (EI) for C₂₈H₂₉FN₂O₅S, found 525 (MH+).

N-Ethyl-4-(4-{[2-ethyl-4-(ethylsulfonyl)-3-fluorophenyl]-carbonyl}-2,3,4,5-tetrahydro-1,4-benzoxazepin-7-yl)benzamide. ¹H NMR (400 MHz, DMSO-d₆): δ 8.58-8.47 (m, 1H), 7.99-7.68 (m, 4H), 7.65-7.46 (m, 2H), 7.33-6.76 (m, 2H), 5.07-4.82 (m, 1H), 4.46 (s, 1H), 4.36-4.04 (m, 3H), 3.64-3.55 (m, 1H), 3.48-3.24 (m, 4H), 2.70-1.96 (m, 2H), 1.21-0.95 (m, 9H). MS (EI) for C₂₉H₃ᵢFN₂O₅S, found 539 (MH+).

4-(4-{[2-Ethyl-3-fluoro-4-(methylsulfonyl)phenyl]carbonyl}-2,3,4,5-tetrahydro-1,4-benzoxazepin-7-yl)-N-methyl-2-(methyloxy)benzamide. ¹H NMR (400 MHz, DMSO-d₆): δ 8.21-8.14 (m, 1H), 7.82-7.71 (m, 2H), 7.66-7.60 (m, 1H), 7.36-7.27 (m, 2H), 7.17-7.07 (m, 3H), 5.07-4.83 (m, 1H), 4.53-3.92 (m, 7H), 3.65-3.53 (m, 1H), 3.37-3.34 (m, 3H), 2.84-2.78 (m, 3H), 2.48-2.38 (m, 2H), 1.13-1.00 (m, 3H). MS (EI) for C₂₈H₂₉FN₂O₆S, found 541 (MH+).

2-Chloro-4-(4-{[2-ethyl-3-fluoro-4-(methylsulfonyl)-phenyl]carbonyl}-2,3,4,5-tetrahydro-1,4-benzoxazepin-7-yl)-N-methylbenzamide. ¹H NMR (400 MHz, DMSO-d₆): δ 8.44-8.36 (m, 1H), 7.85-7.58 (m, 4H), 7.53-7.27 (m, 2H), 7.14-6.94 (m, 2H), 5.07-4.83 (m, 1H), 4.50-4.04 (m, 4H), 3.63-3.56 (m, 1H), 3.36 (s, 3H), 2.77 (t, 3H), 2.72-2.04 (m, 2H), 1.13-0.96 (m, 3H). MS (EI) for C₂₇H₂₆ClFN₂O₅S, found 545 (MH+).

4-(4-{[2-Ethyl-3-fluoro-4-(methylsulfonyl)phenyl]carbonyl}-2,3,4,5-tetrahydro-1,4-benzoxazepin-7-yl)-N-3-dimethylbenzamide. ¹H NMR (400 MHz, DMSO-d₆): δ 8.52-8.38 (m, 1H), 7.81-7.62 (m, 3H), 7.40-7.03 (m, 4H), 5.02-4.76 (m, 1H), 4.52-4.00 (m, 4H), 3.64-3.55 (m, 1H), 3.32 (d, 3H), 2.82-2.75 (m, 3H), 2.73-2.37 (m, 2H), 2.25 (d, 3H), 1.13-0.98 (m, 3H). MS (EI) for C₂₈H₂₉FN₂O₅S, found 525 (MH+).

4-(4-{[2-Ethyl-3-fluoro-4-(methylsulfonyl)phenyl]carbonyl}-2,3,4,5-tetrahydro-1,4-benzoxazepin-7-yl)-N-pyrrolidin-3-ylbenzamide. ¹H NMR (400 MHz, DMSO-d₆): δ 9.18-8.91 (m, 2H), 8.74 (m, 1H), 8.04-7.87 (m, 2H), 7.85-7.70 (m, 2H), 7.67-7.51 (m, 2H), 7.32-6.83 (m, 2H), 5.09-4.81 (m, 1H), 4.68-4.00 (m, 5H), 3.76-3.14 (m, 10H), 2.73-1.96 (m, 2H), 1.14-0.95 (m, 3H). MS (EI) for C₃₀H₃₂FN₃O₅S, found 566 (MH+).

4-(4-{[2-Ethyl-3-fluoro-4-(methylsulfonyl)phenyl]carbonyl}-2,3,4,5-tetrahydro-1,4-benzoxazepin-7-yl)-N-piperidin-3-ylbenzamide. ¹H NMR (400 MHz, DMSO-d₆): δ 9.15-8.77 (m, 2H), 8.60 (m, 1H), 8.04-7.70 (m, 4H), 7.67-7.51 (m, 1H), 7.32-6.84 (m, 2H), 5.09-4.81 (m, 1H), 4.69-4.01 (m, 5H), 3.77-3.42 (m, 3H), 3.36 (m, 3H), 3.19 (m, 1H), 2.95-2.79 (m, 1H), 2.71-2.02 (m, 2H), 1.97-1.57 (m, 4H), 1.13-094 (m, 3H). MS (EI) for C₃₁H₃₄FN₃O₅S, found 580 (MH+).

N-(2,2-Difluoroethyl)-4-(4-{[2-ethyl-3-fluoro-4-(methylsulfonyl)phenyl]carbonyl}-2,3,4,5-tetrahydro-1,4-benzoxazepin-7-yl)benzamide. ¹H NMR (400 MHz, DMSO-d₆): δ 8.98-8.86 (m, 1H), 8.03-7.52 (m, 7H), 7.33-6.83 (m, 2H), 6.31-5.98 (m, 1H), 5.08-4.80 (m, 1H), 4.53-3.99 (m, 4H), 3.79-3.55 (m, 3H), 3.36 (m, 3H), 2.77-2.03 (m, 2H), 1.14-0.95 (m, 3H). MS (EI) for C₂₈H₂₇F₃N₂O₅S, found 561 (MH+).

4-(4-{[2-Ethyl-3-fluoro-4-(methylsulfonyl)phenyl]carbonyl}-2,3,4,5-tetrahydro-1,4-benzoxazepin-7-yl)-N-(2-fluoroethyl)benzamide. ¹H NMR (400 MHz, DMSO-d₆): δ 8.83-8.71 (m, 1H), 8.01-7.78 (m, 5H), 7.66-7.51 (m, 2H), 7.32-6.84 (m, 2H), 5.08-4.79 (m, 1H), 4.66-4.58 (m, 1H), 4.53-3.99 (m, 5H), 3.36 (m, 3H), 2.73-1.99 (m, 2H), 1.13-0.94 (m, 3H). MS (EI) for C₂₈H₂₈F₂N₂O₅S, found 543 (MH+).

4-(4-{[2-ethyl-3-fluoro-4-(methylsulfonyl)phenyl]carbonyl}-2,3,4,5-tetrahydro-1,4-benzoxazepin-7-yl)-N-methylbenzenesulfonamide. ¹H NMR (400 MHz, DMSO-d₆): δ 7.92 (d, 1H), 7.85 (d, 1H), 7.81-7.71 (m, 2.5H), 7.67-7.59 (m, 2H), 7.54-7.48 (m, 1H), 7.29 (d, 0.5H), 7.16-7.10 (m, 1.5H), 6.84 (d, 0.5H), 5.08-4.83 (m, 1H), 4.52-4.00 (m, 4H), 3.65-3.55 (m, 1H), 3.38-3.35 (m, 3H), 2.70-2.60 (m, 0.5H), 2.47-2.24 (m, 4H), 2.12-2.00 (m, 0.5H), 1.10 (t, 1.5H), 0.98 (t, 1.5H). MS (EI) for C₂₆H₂₇FN₂O₆S₂, found 547 (MH+).

[7-(2-{[2-(Dimethylamino)ethyl]amino}pyrimidin-5-yl)-2,3-dihydro-1,4-benzoxazepin-4(5H)-yl][2-ethyl-3-fluoro-4-(methylsulfonyl)phenyl]methanone. ¹H NMR (400 MHz, CDCl₃): δ 8.58 (bs, 1H), 8.50 (s, 1H), 8.30 (s, 1H), 7.87-7.79 (m, 1H), 7.37-7.30 (m, 1H), 7.16-7.04 (m, 2H), 6.66-6.60 (m, 1H), 4.90 (dd, 1H), 4.47-3.95 (m, 4H), 3.75-3.69 (m, 2H), 3.66-3.60 (m, 1H), 3.26 (d, 3H), 2.93 (q, 2H), 2.82-2.30 (m, 2H), 2.55 (d, 6H), 1.18 (tt, 3H). MS (EI) for C₂₇H₃₂FN₅O₄S, found 542 (MH+).

2-[4-(4-{[2-Ethyl-3-fluoro-4-(methylsulfonyl)-phenyl]carbonyl}-2,3,4,5-tetrahydro-1,4-benzoxazepin-7-yl)phenyl]-N-methylacetamide. ¹H NMR (400 MHz, DMSO-d₆): δ 8.05-7.95 (m, 1H), 7.81-7.70 (m, 1H), 7.66 (d, 0.5H), 7.59 (d, 1H), 7.54-7.47 (m, 1H), 7.38-7.24 (m, 3.5H), 7.17 (d, 0.5H), 7.07 (dd, 1H), 6.71 (d, 0.5H), 5.05-4.78 (m, 1H), 4.51-3.95 (m, 4H), 3.63-3.55 (m, 1H), 3.44-3.32 (m, 5H), 2.71-2.55 (m, 3.5H), 2.48-2.39 (m, 0.5H), 2.36-2.25 (m, 0.5H), 2.10-1.98 (m, 0.5H), 1.10 (t, 1.5H), 0.97 (t, 1.5H). MS (EI) for C₂₈H₂₉FN₂O₅S, found 525 (MH+).

4-(4-{[2-Ethyl-3-fluoro-4-(methylsulfonyl)phenyl]carbonyl}-2,3,4,5-tetrahydro-1,4-benzoxazepin-7-yl)-N-(2-morpholin-4-ylethyl)benzamide. ¹H NMR (400 MHz, Methanol-d₄): δ 7.95-7.80 (m, 3H), 7.78-7.71 (m, 1H), 7.61-7.49 (m, 2H), 7.24-7.10 (m, 2H), 6.68 (m, 1H), 5.08-4.83 (m, 1H), 4.47 (s, 1H), 4.39-4.07 (m, 4H), 3.79-3.53 (m, 8H), 3.28 (m, 3H), 2.82-2.08 (m, 7H), 1.23-1.03 (m, 3H). MS (EI) for C₃₂H₃₆FN₃O₆S, found 610 (MH+).

4-(4-{[2-Ethyl-3-fluoro-4-(methylsulfonyl)phenyl]carbonyl}-2,3,4,5-tetrahydro-1,4-benzoxazepin-7-yl)-N-propylbenzamide. ¹H NMR (400 MHz, DMSO-d₆): δ 8.54-8.47 (m, 1H), 7.96-7.83 (dd, 2H), 7.79-7.71 (m, 2.5H), 7.64-7.50 (m, 2H), 7.31-7.07 (m, 2H), 6.86 (d, 0.5H), 5.06-4.81 (m, 1H), 4.52-4.00 (m, 4H), 3.63-3.56 (m, 1H), 3.36 (d, 3H), 3.28-3.17 (m, 2H), 2.74-2.02 (m, 2H), 1.61-1.49 (m, 2H), 1.13-1.05 (m, 3H), 0.90 (dt, 3H). MS (EI) for C₂₉H₃ᵢFN₂O₅S, found 539 (MH+).

3-[4-(4-{[2-Ethyl-3-fluoro-4-(methylsulfonyl)phenyl]-carbonyl}-2,3,4,5-tetrahydro-1,4-benzoxazepin-7-yl)phenyl]-1,1-dimethylurea. ¹H NMR (400 MHz, DMSO-d₆): δ 8.37 (d, 1H), 7.80-7.70 (m, 1H), 7.64 (d, 0.5H), 7.60-7.45 (m, 4H), 7.33-7.26 (m, 1.5H), 7.15 (d, 0.5H), 7.04 (dd, 1H), 6.69 (d, 0.5H), 5.03-4.77 (m, 1H), 4.47-3.98 (m, 4H), 3.65-3.52 (m, 1H), 3.38-3.35 (m, 3H), 2.97-2.92 (m, 6H), 2.69-2.61 (m, 0.5H), 2.47-2.29 (m, 1H), 2.10-2.01 (m, 0.5H), 1.10 (t, 1.5H), 0.99 (t, 1.5H). MS (EI) for C₂₈H₃₀FN₃O₅S, found 540 (MH+).

1-[4-(4-{[2-Ethyl-3-fluoro-4-(methylsulfonyl)phenyl]-carbonyl}-2,3,4,5-tetrahydro-1,4-benzoxazepin-7-yl)phenyl]-3-(2-fluoroethyl)urea. ¹H NMR (400 MHz, DMSO-d₆): δ 8.68 (d, 1H), 7.80-7.70 (m, 1H), 7.62 (d, 0.5H), 7.58-7.40 (m, 4H), 7.33-7.26 (m, 1.5H), 7.15 (d, 0.5H), 7.04 (dd, 1H), 6.66 (d, 0.5H), 6.43-6.34 (m, 1H), 5.03-4.77 (m, 1H), 4.55-3.98 (m, 6H), 3.64-3.52 (m, 1H), 3.48-3.35 (m, 5H), 2.70-2.60 (m, 0.5H), 2.48-2.28 (m, 1H), 2.10-2.00 (m, 0.5H), 1.10 (t, 1.5H), 0.98 (t, 1.5H). MS (EI) for C₂₈H₂₉F₂N₃O₅S, found 558 (MH+).

4-(4-{[2-Ethyl-3-fluoro-4-(methylsulfonyl)phenyl]carbonyl}-2,3,4,5-tetrahydro-1,4-benzoxazepin-7-yl)-3-fluoro-N-methylbenzamide. ¹H NMR (400 MHz, DMSO-d₆): δ 8.64-8.54 (d, 1H), 7.81-7.60 (m, 3H), 7.52-7.09 (m, 4H), 5.05-4.79 (m, 1H), 4.54-3.98 (m, 4H), 3.65-3.55 (m, 1H), 3.34 (d, 3H), 2.80 (m, 3H), 2.74-2.02 (m, 2H), 1.14-0.96 (m, 3H). MS (EI) for C₂₇H₂₆F₂N₂O₅S, found 529 (MH+).

4-(4-{[2-Ethyl-3-fluoro-4-(methylsulfonyl)phenyl]carbonyl}-2,3,4,5-tetrahydro-1,4-benzoxazepin-7-yl)-N-(2,2,2-trifluoroethyl)benzamide. ¹H NMR (400 MHz, DMSO-d₆): δδ 9.21-9.10 (m, 1H), 8.04-7.54 (m, 7H), 7.33-6.85 (m, 2H), 5.12-4.81 (m, 1H), 4.56-3.99 (m, 6H), 3.68-3.52 (m, 1H), 3.36 (m, 3H), 2.75-2.00 (m, 2H), 1.14-0.95 (m, 3H). MS (EI) for C₂₈H₂₆F₄N₂O₅S, found 579 (MH+).

4-(4-{[2-Ethyl-3-fluoro-4-(methylsulfonyl)phenyl]carbonyl}-2,3,4,5-tetrahydro-1,4-benzoxazepin-7-yl)-N-(3,3,3-trifluoropropyl)benzamide. ¹H NMR (400 MHz, DMSO-d₆): δ 8.81-8.68 (m, 1H), 7.97-7.51 (m, 7H), 7.33-6.83 (m, 2H), 5.08-4.81 (m, 1H), 4.53-4.00 (m, 4H), 3.68-3.45 (m, 3H), 3.36 (m, 3H), 2.72-2.03 (m, 4H), 1.15-0.93 (m, 3H). MS (EI) for C₂₉H₂₈F₄N₂O₅S, found 593 (MH+).

4-(4-{[2-Ethyl-3-fluoro-4-(methylsulfonyl)phenyl]carbonyl}-2,3,4,5-tetrahydro-1,4-benzoxazepin-7-yl)-N-(2-methylpropyl)benzamide. ¹H NMR (400 MHz, DMSO-d₆): δ 8.59-8.45 (m, 1H), 7.98-7.83 (m, 2H), 7.81-7.26 (m, 5H), 7.18-6.83 (m, 2H), 5.08-4.79 (m, 1H), 4.53-3.99 (m, 4H), 3.66-3.53 (m, 1H), 3.36 (m, 3H), 3.14-3.06 (m, 2H), 2.71-2.03 (m, 2H), 1.90-1.80 (m, 1H), 1.13-0.95 (m, 3H), 0.93-0.86 (m, 6H). MS (EI) for C₃₀H₃₃FN₂O₅S, found 553 (MH+).

4-(4-{[2-Ethyl-3-fluoro-4-(methylsulfonyl)phenyl]carbonyl}-2,3,4,5-tetrahydro-1,4-benzoxazepin-7-yl)-N-(2,2,2-trifluoro-1-methylethyl)benzamide. ¹H NMR (400 MHz, DMSO-d₆): δ 8.96-8.84 (m, 1H), 8.02-7.89 (m, 2H), 7.85-7.26 (m, 5H), 7.19-6.86 (m, 2H), 5.09-4.81 (m, 2H), 4.53-4.01 (m, 4H), 3.65-3.53 (m, 1H), 3.36 (m, 3H), 2.72-2.05 (m, 2H), 1.42-1.35 (m, 3H), 1.14-0.96 (m, 3H). MS (EI) for C₂₉H₂₈F₄N₂O₅S, found 593 (MH+).

4-(4-{[2-Ethyl-3-fluoro-4-(methylsulfonyl)phenyl]carbonyl}-2,3,4,5-tetrahydro-1,4-benzoxazepin-7-yl)benzamide. ¹H NMR (400 MHz, DMSO-d₆): δ 8.08-7.86 (m, 3H), 7.80-6.84 (m, 6H), 5.08-4.81 (m, 1H), 4.53-3.98 (m, 4H), 3.66-3.53 (m, 1H), 3.36 (m, 3H), 2.70-2.01 (m, 2H), 1.14-0.94 (m, 3H). MS (EI) for C₂₆H₂₅FN₂O₅S, found 497 (MH+).

4-(4-{[2-Ethyl-3-fluoro-4-(methylsulfonyl)phenyl]carbonyl}-2,3,4,5-tetrahydro-1,4-benzoxazepin-7-yl)-N-[(3R)-pyrrolidin-3-yl]benzamide. ¹H NMR (400 MHz, Methanol-d₄): δ 8.02-7.80 (m, 3H), 7.79-7.71 (m, 1H), 7.61-7.51 (m, 2H), 7.24-7.10 (m, 2H), 6.69 (m, 1H), 5.08-4.84 (m, 1H), 4.66-4.59 (m, 1H), 4.47 (s, 1H), 4.38-4.07 (m, 4H), 3.76-3.54 (m, 4H), 3.46-3.36 (m, 2H), 3.29 (m, 3H), 2.80-2.50 (m, 1H), 2.50-2.05 (m, 2H), 1.20-1.04 (m, 3H). MS (EI) for C₃₀H₃₂FN₃O₅S, found 566 (MH+).

N-[2-(Diethylamino)ethyl]-4-(4-{[2-ethyl-3-fluoro-4-(methylsulfonyl)phenyl]carbonyl}-2,3,4,5-tetrahydro-1,4-benzoxazepin-7-yl)benzamide. ¹H NMR (400 MHz, Methanol-d₄): δ 8.00-7.72 (m, 4H), 7.62-7.53 (m, 2H), 7.24-7.11 (m, 3H), 5.08-4.84 (m, 1H), 4.50-4.10 (m, 3H), 3.81-3.74 (m, 2H), 3.45-3.32 (m, 4H), 3.30 (m, 4H), 3.28 (d, 3H), 1.37 (t, 6H), 1.20-1.02 (m, 3H). MS (EI) for C₃₂H₃₈FN₃O₅S, found 596 (MH+).

4-{[2-Ethyl-3-fluoro-4-(methylsulfonyl)phenyl]carbonyl}-7-[4-(piperazin-1-ylcarbonyl)phenyl]-2,3,4,5-tetrahydro-1,4-benzoxazepine. ¹H NMR (400 MHz, DMSO-d₆): δ 9.26 (bs, 2H), 7.81-7.71 (m, 2H), 7.64-7.46 (m, 4H), 7.31-7.08 (m, 3H), 5.06-4.81 (m, 1H), 4.52-3.98 (m, 4H), 3.90-3.44 (m, 6H), 3.36 (d, 3H), 3.25-3.16 (m, 2H), 2.70-2.00 (m, 2H), 1.13-0.94 (m, 3H). MS (EI) for C₃₀H₃₂FN₃O₅S, found 566 (MH+).

4-(4-{[2-Ethyl-3-fluoro-4-(methylsulfonyl)phenyl]carbonyl}-2,3,4,5-tetrahydro-1,4-benzoxazepin-7-yl)-N-[(35)-pyrrolidin-3-yl]benzamide. ¹H NMR (400 MHz, Methanol-d₄): δ 7.85-7.80 (m, 3H), 7.77-7.71 (m, 1H), 7.61-7.49 (m, 2H), 7.24-7.10 (m, 2H), 6.69 (m, 1H), 5.08-4.84 (m, 1H), 4.56-4.49 (m, 1H), 4.47 (s, 1H), 4.38-4.07 (m, 4H), 3.79-3.61 (m, 2H), 3.28 (m, 3H), 3.23-2.94 (m, 3H), 2.77 (m, 1H), 2.60-1.88 (m, 3H), 1.20-1.02 (m, 3H). MS (EI) for C₃₀H₃₂FN₃O₅S, found 566.2 (MH+).

4-(4-{[2-Ethyl-3-fluoro-4-(methylsulfonyl)phenyl]carbonyl}-2,3,4,5-tetrahydro-1,4-benzoxazepin-7-yl)-N-piperidin-4-ylbenzamide. ¹H NMR (400 MHz, DMSO-d₆): δ 8.61-8.48 (m, 3H), 7.96 (d, 1H), 7.88 (d, 1H), 7.81-7.72 (m, 2.5H), 7.64-7.51 (m, 2H), 7.28 (d, 0.5H), 7.17-7.08 (m, 1.5H), 6.85 (d, 0.5H), 5.08-4.81 (m, 1H), 4.52-4.00 (m, 4H), 3.66-3.53 (m, 1H), 3.38-3.28 (m, 5H), 3.08-2.97 (m, 2H), 2.69-2.60 (m, 0.5H), 2.48-2.28 (m, 1H), 2.15-1.93 (m, 2.5H), 1.82-1.68 (m, 2H), 1.10 (t, 1.5H), 0.99 (t, 1.5H). MS (EI) for C₃₁H₃₄FN₃O₅S, found 580 (MH+).

N-Azetidin-3-yl-4-(4-{[2-ethyl-3-fluoro-4-(methylsulfonyl)phenyl]carbonyl}-2,3,4,5-tetrahydro-1,4-benzoxazepin-7-yl)benzamide. ¹H NMR (400 MHz, DMSO-d₆): δ 9.25-9.18 (m, 1H), 8.84 (br s, 2H), 8.03-7.72 (m, 4.5H), 7.67-7.56 (m, 2H), 7.28 (d, 0.5H), 7.16-7.09 (m, 1.5H), 6.86 (d, 0.5H), 5.11-4.77 (m, 2H), 4.51-4.02 (m, 8H), 3.64-3.57 (m, 1H), 3.36 (m, 3H), 2.70-2.60 (m, 0.5H), 2.46-2.26 (m, 1H), 2.15-2.04 (m, 0.5H), 1.10 (t, 1.5H), 0.99 (t, 1.5H). MS (EI) for C₂₉H₃₀FN₃O₅S, found 552 (MH+).

4-(4-{[2-Ethyl-3-fluoro-4-(methylsulfonyl)phenyl]carbonyl}-2,3,4,5-tetrahydro-1,4-benzoxazepin-7-yl)-N-(piperidin-2-ylmethyl)benzamide. ¹H NMR (400 MHz, DMSO-d₆): δ 8.87-8.78 (m, 1H), 8.75-8.65 (m, 1H), 8.57-8.44 (m, 1H), 8.01 (d, 1H), 7.94 (d, 1H), 7.85-7.72 (m, 2.5H), 7.66-7.54 (m, 2H), 7.28 (d, 0.5H), 7.16-7.08 (m, 1.5H), 6.88 (d, 0.5H), 5.08-4.82 (m, 1H), 4.53-4.02 (m, 4H), 3.65-3.40 (m, 2H), 3.38-3.34 (m, 3H), 3.30-3.17 (m, 2H), 2.91-2.80 (m, 1H), 2.70-2.61 (m, 0.5H), 2.48-2.04 (m, 1.5H), 1.91-1.40 (m, 6H), 1.09 (t, 1.5H), 0.99 (t, 1.5H). MS (EI) for C₃₂H₃₆FN₃O₅S, found 594 (MH+).

4-(4-{[2-Ethyl-3-fluoro-4-(methylsulfonyl)phenyl]carbonyl}-2,3,4,5-tetrahydro-1,4-benzoxazepin-7-yl)-N-(pyrrolidin-3-ylmethyl)benzamide. ¹H NMR (400 MHz, Methanol-d₄): δ 7.95-7.79 (m, 3H), 7.76-7.70 (m, 1H), 7.60-7.48 (m, 2H), 7.23-7.10 (m, 2H), 6.67 (m, 1H), 5.08-4.84 (m, 1H), 4.46 (s, 1H), 4.39-4.06 (m, 4H), 3.76-3.37 (m, 6H), 3.35-3.24 (m, 3H), 3.20-2.50 (m, 4H), 2.50-1.60 (m, 2H), 1.20-1.02 (m, 3H). MS (EI) for C₃₁H₃₄FN₃O₅S, found 580.2 (MH+).

4-(4-{[2-Ethyl-3-fluoro-4-(methylsulfonyl)phenyl]carbonyl}-2,3,4,5-tetrahydro-1,4-benzoxazepin-7-yl)-N-(piperidin-3-ylmethyl)benzamide. ¹H NMR (400 MHz, DMSO-d₆): δ 9.00-8.50 (m, 2H), 7.99-7.85 (dd, 2H), 7.83-7.71 (m, 2H), 7.66-7.52 (m, 2H), 7.39-7.02 (m, 3H), 5.07-4.81 (m, 1H), 4.58-3.96 (m, 4H), 3.64-3.55 (m, 1H), 3.36 (d, 3H), 3.32-3.12 (m, 4H), 2.85-2.55 (m, 2H), 2.38-1.96 (m, 3H), 1.85-1.73 (m, 2H), 1.69-1.54 (m, 1H), 1.30-1.16 (m, 1H), 1.14-0.94 (m, 3H). MS (EI) for C₃₂H₃₆FN₃O₅S, found 594 (MH+).

4-(4-{[2-Ethyl-3-fluoro-4-(methylsulfonyl)phenyl]carbonyl}-2,3,4,5-tetrahydro-1,4-benzoxazepin-7-yl)-N-(1-methylazetidin-3-yl)benzamide. ¹H NMR (400 MHz, DMSO-d₆): δ 10.00 (br s, 1H), 9.25-9.17 (m, 1H), 8.02-7.72 (m, 4.5H), 7.67-7.56 (m, 2H), 7.28 (d, 0.5H), 7.16-7.08 (m, 1.5H), 6.86 (d, 0.5H), 5.07-4.69 (m, 2H), 4.53-4.01 (m, 8H), 3.65-3.54 (m, 1H), 2.93-2.87 (m, 3H), 2.70-2.03 (m, 2H), 1.09 (t, 1.5H), 0.99 (t, 1.5H). MS (EI) for C₃₀H₃₂FN₃O₅S, found 566 (MH+).

4-(4-{[2-Ethyl-3-fluoro-4-(methylsulfonyl)phenyl]carbonyl}-2,3,4,5-tetrahydro-1,4-benzoxazepin-7-yl)-N-(pyrrolidin-2-ylmethyl)benzamide. ¹H NMR (400 MHz, Methanol-d₄): δ 7.95-7.79 (m, 3H), 7.76-7.70 (m, 1H), 7.60-7.48 (m, 2H), 7.23-7.09 (m, 2H), 6.67 (m, 1H), 5.07-4.83 (m, 1H), 4.46 (s, 1H), 4.38-4.05 (m, 4H), 3.76-3.38 (m, 6H), 3.28 (m, 3H), 2.18-1.52 (m, 2H), 1.20-1.02 (m, 3H). MS (EI) for C₃₁H₃₄FN₃O₅S, found 580 (MH+).

N-(Azetidin-3-ylmethyl)-4-(4-{[2-ethyl-3-fluoro-4-(methylsulfonyl)phenyl]carbonyl}-2,3,4,5-tetrahydro-1,4-benzoxazepin-7-yl)benzamide. ¹H NMR (400 MHz, DMSO-d₆): δ 8.84-8.50 (m, 2H), 8.00-7.86 (dd, 2H), 7.83-7.71 (m, 2.5H), 7.65-7.53 (m, 2H), 7.31-7.08 (m, 2H), 6.86 (d, 0.5H), 5.07-4.83 (m, 1H), 4.52-4.02 (m, 4H), 4.01-3.91 (m, 2H), 3.86-3.75 (m, 2H), 3.65-3.55 (m, 1H), 3.54-3.45 (m, 2H), 3.36 (d, 3H), 3.10-2.97 (m, 1H), 2.70-2.00 (m, 2H), 1.14-0.95 (m, 3H). MS (EI) for C₃₀H₃₂FN₃O₅S, found 566 (MH+).

4-(4-{[2-Ethyl-3-fluoro-4-(methylsulfonyl)phenyl]carbonyl}-2,3,4,5-tetrahydro-1,4-benzoxazepin-7-yl)-N-[2-(methylamino)ethyl]benzamide. ¹H NMR (400 MHz, DMSO-d₆): δ 8.80 (bs, 1H), 8.70 (bs, 1H), 8.05-7.90 (dd, 2H), 7.85-7.71 (m, 2.5H), 7.67-7.54 (m, 2H), 7.31-7.07 (m, 2H), 6.87 (d, 0.5H), 5.07-4.82 (m, 1H), 4.57-4.02 (m, 4H), 3.67-3.53 (m, 1H), 3.36 (d, 3H), 3.17-3.05 (m, 1H), 2.60 (m, 4H), 2.40-2.03 (m, 1H), 1.13-0.95 (m, 3H). MS (EI) for C₂₉H₃₂FN₃O₅S, found 544 (MH+).

4-(4-{[2-Ethyl-4-(ethylsulfonyl)-3-fluorophenyl]carbonyl}-2,3,4,5-tetrahydro-1,4-benzoxazepin-7-yl)-N—[(R3R)-pyrrolidin-3-yl]benzamide. ¹H NMR (400 MHz, DMSO-d₆): δ 9.24-9.00 (m, 2H), 8.75 (m, 1H), 8.01-7.88 (m, 2H), 7.82-7.48 (m, 4H), 7.30-6.77 (m, 2H), 5.05-4.83 (m, 1H), 4.54 (m, 1H), 4.44 (s, 1H), 3.73-3.32 (m, 6H), 3.28-3.14 (m, 2H), 2.69-1.95 (m, 4H), 1.13-0.91 (m, 6H). MS (EI) for C₃₁H₃₄FN₃O₅S, found 580 (MH+).

4-(4-{[2-Ethyl-4-(ethylsulfonyl)-3-fluorophenyl]carbonyl}-2,3,4,5-tetrahydro-1,4-benzoxazepin-7-yl)-N-(pyrrolidin-3-ylmethyl)benzamide. ¹H NMR (400 MHz, DMSO-d₆): δ 9.14-9.00 (s, 2H), 8.82-8.72 (m, 1H), 8.00-7.86 (m, 2H), 7.83-7.67 (m, 2H), 7.65-7.49 (m, 2H), 7.32-678 (m, 2H), 5.09-4.81 (m, 1H), 4.46 (s, 1H), 4.29 (m, 1H), 4.23-4.04 (m, 2H), 3.75-3.05 (m, 10H), 2.97-2.86 (m, 1H), 2.72-1.94 (m, 3H), 1.22-0.95 (m, 6H). MS (EI) for C₃₂H₃₆FN₃O₅S, found 595 (MH+).

4-(4-{[2-Ethyl-3-fluoro-4-(methylsulfonyl)phenyl]carbonyl}-2,3,4,5-tetrahydro-1,4-benzoxazepin-7-yl)-N-(morpholin-2-ylmethyl)benzamide. ¹H NMR (400 MHz, DMSO-d₆): δ 9.17-8.99 (m, 2H), 8.79-8.71 (m, 1H), 7.97 (d, 1H), 7.89 (d, 1H), 7.82-7.72 (m, 2.5H), 7.65-7.53 (m, 2H), 7.28 (d, 0.5H), 7.17-7.08 (m, 1.5H), 6.86 (d, 0.5H), 5.07-4.82 (m, 1H), 4.52-3.56 (m, 9H), 3.36 (s, 3H), 3.32-3.24 (m, 1H), 3.21-3.13 (m, 1H), 3.06-2.94 (m, 1H), 2.85-2.74 (m, 1H), 2.70-2.04 (m, 2H), 1.09 (t, 1.5H), 0.99 (t, 1.5H). MS (EI) for C₃₁H₃₄FN₃O₆S, found 596 (MH+).

4-(4-{[2-Ethyl-3-fluoro-4-(methylsulfonyl)phenyl]carbonyl}-2,3,4,5-tetrahydro-1,4-benzoxazepin-7-yl)-N-[(3aR,5 r,6aS)-octahydrocyclopenta[c]pyrrol-5-ylmethyl]benzamide. ¹H NMR (400 MHz, DMSO-d₆): δ 8.98 (br s, 2H), 8.60-8.52 (m, 1H), 7.94 (d, 1H), 7.86 (d, 1H), 7.80-7.72 (m, 2.5H), 7.64-7.50 (m, 2H), 7.28 (d, 0.5H), 7.16-7.07 (m, 1.5H), 6.85 (d, 0.5H), 5.07-4.81 (m, 1H), 4.51-4.01 (m, 4H), 3.39-3.34 (m, 3H), 3.32-3.26 (m, 2H), 3.16-3.00 (m, 4H), 2.81-2.59 (m, 1H), 2.47-1.97 (m, 5H), 1.21-0.95 (m, 5H). MS (EI) for C₃₄H₃₈FN₃O₅S, found 620 (MH+).

4-(4-{[2-Ethyl-3-fluoro-4-(methylsulfonyl)phenyl]carbonyl}-2,3,4,5-tetrahydro-1,4-benzoxazepin-7-yl)-N-[(3R)-pyrrolidin-3-ylmethyl]benzamide. ¹H NMR (400 MHz, DMSO-d₆): δ 8.98 (br s, 2H), 8.79-8.71 (m, 1H), 7.96 (d, 1H), 7.88 (d, 1H), 7.82-7.72 (m, 2.5H), 7.64-7.52 (m, 2H), 7.28 (d, 0.5H), 7.17-7.05 (m, 1.5H), 6.86 (d, 0.5H), 5.07-4.82 (m, 1H), 4.51-4.00 (m, 4H), 3.40-3.07 (m, 9H), 2.97-2.87 (m, 1H), 2.69-1.95 (m, 3H), 1.73-1.62 (m, 1H), 1.10 (t, 1.5H), 0.99 (t, 1.5H). MS (EI) for C₃₁H₃₄FN₃O₅S, found 580 (MH+).

[7-(6-Aminopyridin-3-yl)(5,5⁻²H₂)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.24-8.23 (d, 0.5H), 8.04-8.03 (d, 0.5H), 7.78-7.71 (m, 1H), 7.71-7.68 (dd, 0.5H), 7.57-7.57 (d, 0.5H), 7.44-7.39 (m, 1.5H), 7.28-7.26 (d, 0.5H), 7.16-7.14 (dd, 0.5H), 7.04-7.01 (dd, 1H), 6.66-6.65 (d, 0.5H), 6.53-6.45 (dd, 1H), 6.06 (s, 2H), 4.29-3.53 (m, 4H), 3.38-3.36 (d, 3H), 2.69-2.02 (m, 2H), 1.12-0.97 (d, 3H). MS (EI) for C₂₄H₂₂FN₃O₄SD₂, found 472 (MH+).

[7-(6-Aminopyridin-3-yl)(2,2,3,3,5,5-²H₆)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.24-8.23 (d, 0.5H), 8.04-8.03 (d, 0.5H), 7.78-7.71 (m, 1H), 7.71-7.68 (dd, 0.5H), 7.57-7.56 (d, 0.5H), 7.44-7.39 (m, 1.5H), 7.28-7.26 (d, 0.5H), 7.16-7.14 (dd, 0.5H), 7.03-7.01 (dd, 1H), 6.65-6.65 (d, 0.5H), 6.53-6.45 (dd, 1H), 6.05 (s, 2H), 3.38-3.36 (d, 3H), 2.69-2.02 (m, 2H), 1.11-0.97 (d, 3H). MS (EI) for C₂₄H₁₈FN₃O₄SD₆, found 476 (MH+).

[7-(6-Aminopyridin-3-yl)(2,2-²H₂)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.24-8.23 (d, 0.5H), 8.04-8.03 (d, 0.5H), 7.78-7.71 (m, 1H), 7.71-7.68 (dd, 0.5H), 7.57-7.56 (d, 0.5H), 7.45-7.39 (m, 1.5H), 7.28-7.26 (d, 0.5H), 7.16-7.14 (dd, 0.5H), 7.03-7.01 (dd, 1H), 6.65-6.65 (d, 0.5H), 6.53-6.45 (dd, 1H), 6.05 (s, 2H), 5.00-4.34 (m, 2H), 4.13-3.52 (m, 2H), 3.38-3.36 (d, 3H), 2.69-2.02 (m, 2H), 1.11-0.97 (d, 3H). MS (EI) for C₂₄H₂₂FN₃O₄SD₂, found 472 (MH+).

[7-(6-Aminopyridin-3-yl)(3,3-²H₂)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.24-8.23 (d, 0.5H), 8.04-8.03 (d, 0.5H), 7.78-7.71 (m, 1H), 7.71-7.68 (dd, 0.5H), 7.57-7.57 (d, 0.5H), 7.45-7.39 (m, 1.5H), 7.28-7.26 (d, 0.5H), 7.16-7.14 (dd, 0.5H), 7.04-7.01 (dd, 1H), 6.65-6.65 (d, 0.5H), 6.53-6.45 (dd, 1H), 6.05 (s, 2H), 5.00-4.33 (m, 2H), 4.27-4.04 (m, 2H), 3.38-3.35 (d, 3H), 2.69-2.02 (m, 2H), 1.11-0.97 (d, 3H). MS (EI) for C₂₄H₂₂FN₃O₄SD₂, found 472 (MH+).

4-{[7-(2-Amino-1,3-thiazol-5-yl)-2,3-dihydro-1,4-benzoxazepin-4(5H)-yl]carbonyl}-3-ethyl-N-(1-methylethyl)benzenesulfonamide. ¹H NMR (400 MHz, DMSO-d₆): δ 7.74 (s, 1H), 7.71-7.34 (m, 3H), 7.30-6.35 (m, 5H), 4.97-4.68 (m, 1H), 4.39-3.94 (m, 4H), 3.58-3.48 (m, 1H), 3.26 (s, 1H), 2.64-2.17 (m, 2H), 1.15-0.90 (m, 9H). MS (EI) for C₂₄H₂₈N₄O₄S₂, found 501 (MH+).

N-(5-{4-[(2-Ethyl-4-{[(1-methylethyl)amino]sulfonyl}-phenyl)carbonyl]-2,3,4,5-tetrahydro-1,4-benzoxazepin-7-yl}-1,3-thiazol-2-yl)acetamide. ¹H NMR (400 MHz, DMSO-d₆): δ 7.86-7.40 (m, 5H), 7.38-6.54 (m, 2H), 5.01-4.75 (m, 1H), 4.46-3.98 (m, 4H), 3.58-3.48 (m, 1H), 3.33-3.20 (m, 1H), 2.68-2.21 (m, 2H), 2.20-2.11 (m, 3H), 1.15-0.90 (m, 9H). MS (EI) for C₂₆H₃₀N₄O₅S₂, found 543 (MH+).

3-Ethyl-N-(1-methylethyl)-4-({7-[4-(1H-pyrazol-3-yl)phenyl]-2,3-dihydro-1,4-benzoxazepin-4(5H)-yl}carbonyl)benzenesulfonamide. ¹H NMR (400 MHz, DMSO-d₆): δ 7.95-7.34 (m, 9H), 730-6.54 (m, 3H), 5.08-4.75 (m, 1H), 4.49-3.94 (m, 4H), 3.62-3.42 (m, 1H), 3.35-3.20 (m, 1H), 2.66-2.16 (m, 2H), 1.17-0.87 (m, 9H). MS (EI) for C₃₀H₃₂N₄O₄S, found 545 (MH+).

4-{[2-Ethyl-3-fluoro-4-(methylsulfonyl)phenyl]carbonyl}-7-[4-(1H-imidazol-2-yl)phenyl]-2,3,4,5-tetrahydro-1,4-benzoxazepine. ¹H NMR (400 MHz, DMSO-d₆): δ 8.24 (d, 1H), 8.15 (d, 1H), 8.01 (d, 1H), 7.87 (d, 0.5H), 7.84 (d, 2H), 7.79-7.66 (m, 3H), 7.32-7.08 (m, 2H), 6.99 (d, 0.5H), 5.09-4.85 (m, 1H), 4.56-4.00 (m, 4H), 3.68-3.51 (m, 1H), 3.37 (d, 3H), 2.74-2.04 (m, 2H), 1.14-0.96 (m, 3H). MS (EI) for C₂₈H₂₆FN₃O₄S, found 520 (MH+).

4-{[2-Ethyl-3-fluoro-4-(methylsulfonyl)phenyl]carbonyl}-7-[4-(1H-imidazol-4-yl)phenyl]-2,3,4,5-tetrahydro-1,4-benzoxazepine. ¹H NMR (400 MHz, DMSO-d₆): δ 12.21 (bs, 1H), 7.90-7.52 (m, 7.5H), 7.43 (d, 1H), 7.31-7.14 (dd, 1H), 7.08 (dd, 1H), 6.79 (m, 0.5H), 5.06-4.80 (m, 1H), 4.50-3.98 (m, 4H), 3.67-3.55 (m, 1H), 2.72-2.00 (m, 2H), 1.14-0.95 (m, 3H). MS (EI) for C₂₈H₂₆FN₃O₄S, found 520 (MH+).

4-{[2-Ethyl-3-fluoro-4-(methylsulfonyl)phenyl]carbonyl}-7-(1H-pyrazol-4-yl)-2,3,4,5-tetrahydro-1,4-benzoxazepine. ¹H NMR (400 MHz, Methanol-d₄): δ 7.95 (s, 1H), 7.89-7.79 (m, 1H), 7.75 (S, 1H), 7.49-7.42 (m, 1H), 7.22-7.14 (m, 1H), 7.05-7.00 (m, 1H), 6.57 (m, 1H), 5.02-4.77 (m, 1H), 4.45-4.12 (m, 4H), 3.70-3.62 (m, 1H), 3.26 (s, 3H), 1.20-1.02 (m, 3H). MS (EI) for C₂₂H₂₂FN₃O₄S, found 444 (MH+).

4-{[2-Ethyl-3-fluoro-4-(methylsulfonyl)phenyl]carbonyl}-7-[4-(1H-pyrazol-4-yl)phenyl]-2,3,4,5-tetrahydro-1,4-benzoxazepine. ¹H NMR (400 MHz, DMSO-d₆): δ 8.20-8.05 (m, 2H), 7.85-7.51 (m, 5.5H), 7.46-7.41 (m, 1H), 7.29-7.17 (dd, 1H), 7.08 (dd, 1H), 6.80 (d, 0.5H), 5.07-4.79 (m, 1H), 4.53-3.97 (m, 4H), 3.70-3.54 (m, 1H), 3.36 (d, 3H), 2.72-2.00 (m, 2H), 1.14-0.95 (m, 3H). MS (EI) for C₂₈H₂₆FN₃O₄S, found 520 (MH+).

5-[4-(4-{[2-Ethyl-3-fluoro-4-(methylsulfonyl)phenyl]-carbonyl}-2,3,4,5-tetrahydro-1,4-benzoxazepin-7-yl)phenyl]-1H-pyrazol-3-amine. ¹H NMR (400 MHz, DMSO-d₆): δ 11.75 (bs, 1H), 7.82-7.64 (m, 5.5H), 7.56 (m, 1H), 7.44 (d, 1H), 7.29-7.16 (dd, 1H), 7.08 (dd, 1H), 6.79 (d, 0.5H), 5.77 (d, 1H), 5.07-4.83 (m, 1H), 4.81 (bs, 1H), 4.50-4.08 (m, 4H), 3.67-3.57 (m, 1H), 3.37 (d, 3H), 2.71-2.00 (m, 2H), 1.15-0.92 (m, 3H). MS (EI) for C₂₈H₂₇FN₄O₄S, found 535 (MH+).

4-{[2-Ethyl-4-(ethylsulfonyl)-3-fluorophenyl]carbonyl}-7-[4-(1H-pyrazol-1-yl)phenyl]-2,3,4,5-tetrahydro-1,4-benzoxazepine. ¹H NMR (400 MHz, DMSO-d₆): δ 8.61-8.51 (m, 1H), 8.00-7.91 (m, 7H), 7.16-6.53 (m, 3H), 5.07-4.80 (m, 1H), 4.45 (s, 1H), 4.37-4.05 (m, 3H), 3.60 (m, 1H), 3.47-3.35 (m, 2H), 2.70-2.08 (m, 2H), 1.20-0.95 (m, 6H). MS (EI) for C₂₉H₂₈FN₃O₄S, found 534 (MH+).

5-[4-(4-{[2-Ethyl-4-(ethylsulfonyl)-3-fluorophenyl]carbonyl}-2,3,4,5-tetrahydro-1,4-benzoxazepin-7-yl)phenyl]-1,3,4-thiadiazol-2-amine. ¹H NMR (400 MHz, DMSO-d₆): δ 7.88-7.69 (m, 5H), 7.64-7.27 (m, 4H), 7.15-6.74 (m, 2H), 5.08-4.81 (m, 1H), 4.45 (s, 1H), 4.34-4.06 (m, 3H), 3.66-3.55 (m, 1H), 3.46-3.35 (m, 2H), 2.69-2.05 (m, 2H), 1.20-0.95 (m, 6H). MS (EI) for C₂₈H₂₇FN₄O₄S₂, found 567 (MH+).

4-{[2-Ethyl-3-fluoro-4-(methylsulfonyl)phenyl]carbonyl}-7-[4-(4H-1,2,4-triazol-3-yl)phenyl]-2,3,4,5-tetrahydro-1,4-benzoxazepine. ¹H NMR (400 MHz, DMSO-d₆): δ 8.64 (bs, 1H), 8.22-7.98 (m, 2.5H), 7.95 (s, 1H), 7.84-7.48 (m, 4H), 7.33-7.07 (m, 2H), 6.81 (m, 0.5H), 5.08-4.80 (m, 1H), 4.52-4.0 (m, 4H), 3.67-3.55 (m, 1H), 3.37 (d, 3H), 3.20-2.00 (m, 2H), 1.14-0.96 (m, 3H). MS (EI) for C₂₇H₂₅FN₄O₄S, found 521 (MH+).

4-{[2-Ethyl-3-fluoro-4-(methylsulfonyl)phenyl]carbonyl}-7-[4-(2-methyl-1H-imidazol-4-yl)phenyl]-2,3,4,5-tetrahydro-1,4-benzoxazepine. ¹H NMR (400 MHz, DMSO-d₆): δ 12.05 (d, 1H), 11.83 (bs, 1H), 7.86-7.68 (m, 4H), 7.67-7.34 (m, 2.5H), 7.32-7.07 (m, 2H), 6.77 (s, 0.5H), 5.06-4.78 (m, 1H), 4.51-3.95 (m, 4H), 3.67-3.52 (m, 1H), 3.37 (d, 3H), 2.72-2.34 (m, 1H), 2.32 (s, 3H), 2.13-2.02 (m, 1H), 1.14-0.96 (m, 3H). MS (EI) for C₂₉H₂₈FN₃O₄S, found 534 (MH+).

4-{[2-Ethyl-3-fluoro-4-(methylsulfonyl)phenyl]carbonyl}-7-{4-[3-(trifluoromethyl)-1H-pyrazol-5-yl]phenyl}-2,3,4,5-tetrahydro-1,4-benzoxazepine. ¹H NMR (400 MHz, DMSO-d₆): δ 7.97-7.91 (m, 1H), 7.90-7.71 (m, 4H), 7.66-7.56 (m, 2H), 7.32-7.14 (m, 1.5H), 7.12-7.07 (m, 1H), 6.90 (m, 0.5H), 5.07-4.82 (m, 1H), 4.53-3.98 (m, 4H), 3.67-3.55 (m, 1H), 3.37 (d, 3H), 2.72-2.30 (m, 2H), 1.14-0.95 (m, 3H). MS (EI) for C₂₉H₂₅F₄N₃O₄S, found 588 (MH+).

7-[4-(4,5-Dihydro-1H-imidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, CDCl₃): δ 7.90-7.78 (m, 3H), 7.71-7.39 (m, 3H), 7.18-7.04 (m, 2H), 6.63 (s, 1H), 5.12-4.71 (m, 1H), 4.48-3.94 (m, 4H), 3.81 (m, 4H), 3.66-3.60 (m, 1H), 3.24 (s, 3H), 2.82-2.22 (m, 2H), 1.32-1.08 (m, 3H). MS (EI) for C₂₈H₂₈FN₃O₄S, found 522 (MH+).

4-{[2-Ethyl-3-fluoro-4-(methylsulfonyl)phenyl]carbonyl}-7-{4-[2-(trifluoromethyl)-1H-imidazol-5-yl]phenyl}-2,3,4,5-tetrahydro-1,4-benzoxazepine. ¹H NMR (400 MHz, DMSO-d₆): δ 10.62-10.15 (d, 1H), 7.81-7.38 (m, 5H), 7.27-7.15 (m, 2H), 7.06 (dd, 1H), 6.99-6.78 (m, 2H), 5.04-4.88 (m, 1H), 4.59-3.94 (m, 4H), 3.77-3.47 (m, 1H), 3.42 (m, 3H), 2.72-1.95 (m, 2H), 1.15-0.96 (m, 3H). MS (EI) for C₂₉H₂₅F₄N₃O₄S, found 588 (MH+).

4-[4-(4-{[2-Ethyl-3-fluoro-4-(methylsulfonyl)phenyl]-carbonyl}-2,3,4,5-tetrahydro-1,4-benzoxazepin-7-yl)phenyl]-1,3-dihydro-2H-imidazol-2-one. ¹H NMR (400 MHz, DMSO-d₆): δ 10.57 (s, 1H), 10.10 (s, 1H), 7.79-6.56 (m, 10H), 5.05-3.85 (m, 5H), 3.65-3.53 (m, 1H), 3.38 (s, 3H), 2.70-2.04 (m, 2H), 1.12-0.95 (m, 3H). MS (EI) for C₂₈H₂₆FN₃O₅S, found 536 (MH+).

4-{[2-Ethyl-3-fluoro-4-(methylsulfonyl)phenyl]carbonyl}-7-[4-(4-methyl-4,5-dihydro-1H-imidazol-2-yl)phenyl]-2,3,4,5-tetrahydro-1,4-benzoxazepine. ¹H NMR (400 MHz, CDCl₃): δ 7.90-7.78 (m, 3H), 7.70-7.35 (m, 3H), 7.17-7.03 (m, 2H), 6.62 (s, 1H), 5.75 (bs, 1H), 5.12-4.71 (m, 1H), 4.44-3.88 (m, 6H), 3.67-3.59 (m, 1H), 3.45-3.35 (m, 1H), 3.23 (m, 3H), 2.84-2.20 (m, 2H), 1.40-1.05 (m, 6H). MS (EI) for C₂₉H₃₀FN₃O₄S, found 536 (MH+).

4-{[2-Ethyl-4-(ethylsulfonyl)-3-fluorophenyl]carbonyl}-7-[4-(5-methyl-4H-1,2,4-triazol-3-yl)phenyl]-2,3,4,5-tetrahydro-1,4-benzoxazepine. ¹H NMR (400 MHz, DMSO-d₆): δ 8.10-7.96 (m, 2H), 7.82-6.69 (m, 2H), 7.64-7.47 (m, 2H), 7.30 (m, 1H), 7.15-7.07 (m, 2H), 6.76 (m, 1H), 5.09-4.82 (m, 1H), 4.50-4.06 (m, 3H), 4.64-3.55 (m, 1H), 3.42 (m, 3H), 2.70-2.52 (m, 1H), 2.40 (s, 3H), 2.30-2.05 (m, 1H), 1.20-0.96 (m, 6H). MS (EI) for C₂₉H₂₉FN₄O₄S, found 549 (MH+).

7-[4-(5-Chloro-1H-imidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 8.00-7.88 (dd, 2H), 7.82-7.71 (m, 2H), 7.66-7.51 (m, 2H), 7.42-7.06 (m, 3H), 6.84 (m, 1H), 5.08-4.80 (m, 1H), 4.55-3.93 (m, 4H), 3.64-3.53 (m, 1H), 3.38 (d, 3H), 2.74-2.02 (m, 2H), 1.14-0.96 (m, 3H). MS (EI) for C₂₈H₂₅ClFN₃O₄S, found 554 (MH+).

4-{[2-Ethyl-4-(ethylsulfonyl)-3-fluorophenyl]carbonyl}-7-{4-[2-(trifluoromethyl)-1H-imidazol-5-yl]phenyl}-2,3,4,5-tetrahydro-1,4-benzoxazepine. ¹H NMR (400 MHz, CDCl₃): δ 11.6-10.5 (m, 1H), 7.90-7.76 (m, 2H), 7.66-7.36 (m, 5H), 7.20-7.05 (m, 2H), 6.65-6.50 (m, 1H), 5.15-4.70 (m, 0.5H), 4.47-4.05 (m, 4H), 4.04-3.94 (m, 0.5H), 3.72-3.58 (m, 1H), 3.45-3.28 (m, 2H), 2.84-2.16 (m, 2H), 1.45-1.08 (m, 6H). MS (EI) for C₃₀H₂₇F₄N₃O₄S, found 602 (MH+).

4-{[2-Ethyl-3-fluoro-4-(methylsulfonyl)phenyl]carbonyl}-7-[4-(4-phenyl-1H-imidazol-2-yl)phenyl]-2,3,4,5-tetrahydro-1,4-benzoxazepine. ¹H NMR (400 MHz, DMSO-d₆): δ 12.73 (s, 1H), 8.20-7.70 (m, 7H), 7.67-7.54 (m, 2H), 7.47-7.33 (m, 2H), 7.30-7.08 (m, 2H), 6.87 (m, 1H), 5.08-4.82 (m, 1H), 4.54-4.00 (m, 4H), 3.64-3.57 (m, 1H), 3.37 (d, 3H), 2.71-2.31 (m, 2H), 1.15-0.97 (m, 3H). MS (EI) for C₃₄H₃₀FN₃O₄S, found 596 (MH+).

[7-(6-Aminopyridin-3-yl)(2,2,3,3,5,5-²H₆)-2,3-dihydro-1,4-benzoxazepin-4(5H)-yl][2-(methyl)phenyl]methanone. ¹H NMR (400 MHz, CDCl₃): δ 8.24-7.95 (m, 1H), 7.71-7.19 (m, 5.5H), 7.12-6.99 (m, 2H), 6.53-6.40 (m, 1.5H), 2.12-1.85 (d, 3H). MS (EI) for C₂₂H₁₅N₃O₂D₆, found 366 (MH+).

4-{[2-Ethyl-3-fluoro-4-(methylsulfonyl)phenyl]carbonyl}-7-[4-(2-phenyl-1H-imidazol-5-yl)phenyl]-2,3,4,5-tetrahydro-1,4-benzoxazepine. ¹H NMR (400 MHz, DMSO-d₆): δ 12.71 (bs, 1H), 8.14-7.66 (m, 7H), 7.64-7.34 (m, 3H), 7.29 (d, 1H), 7.17 (d, 1H), 7.09 (m, 2H), 6.82 (m, 1H), 5.08-4.96 (m, 1H), 4.53-4.00 (m, 4H), 3.65-3.55 (m, 1H), 3.37 (s, 3H), 2.90-2.02 (m, 2H), 1.17-0.95 (m, 3H). MS (EI) for C₃₄H₃₀FN₃O₄S, found 596 (MH+).

7-[4-(5-Fluoro-1H-benzimidazol-2-yl)phenyl]-4-{[3-fluoro-2-methyl-4-(methylsulfonyl)phenyl]carbonyl}-2,3,4,5-tetrahydro-1,4-benzoxazepine. ¹H NMR (400 MHz, DMSO-d₆): δ 8.33-8.17 (m, 2H), 7.94-7.60 (m, 6H), 7.50-7.05 (m, 4H), 6.88 (m, 1H), 4.97 (m, 1H), 4.58-4.14 (m, 4H), 4.08 (m, 1H), 3.60 (s, 3H), 2.16-1.78 (m, 3H). MS (EI) for C₃₁H₂₅F₂N₃O₄S, found 574 (MH+).

4-{[2-Ethyl-3-fluoro-4-(methylsulfonyl)phenyl]carbonyl}-7-{4-[4-(phenylmethyl)piperazin-1-yl]phenyl}-2,3,4,5-tetrahydro-1,4-benzoxazepine. ¹H NMR (400 MHz, CDCl₃): δ 7.89 (t, 1H), 7.82 (t, 1H), 7.57 (m, 1H), 7.36 (m, 2H), 7.08-6.92 (m, 4H), 6.60 (d, 1H), 4.94 (d, 1H), 4.67 (d, 1H), 4.40-3.91 (m, 8H), 3.60 (m, 2H), 3.28 (s, 3H), 3.24 (s, 3H), 2.75 (m, 1H), 2.65 (m, 1H), 2.52 (m, 1H), 2.34 (m, 1H), 1.17 (m, 6H). MS (EI) for C₃₆H₃₈FN₃O₄S, found 628 (MH+).

4-{[2-Ethyl-3-fluoro-4-(methylsulfonyl)phenyl]carbonyl}-7-(4-morpholin-4-ylphenyl)-2,3,4,5-tetrahydro-1,4-benzoxazepine. ¹H NMR (400 MHz, DMSO-d₆): δ 7.72 (m, 1H), 7.58, 6.63 (m, 1H), 7.54 (d, 1H), 7.43 (m, 1H), 7.30-7.14 (m, 2H), 7.00 (m, 2H), 6.92 (m, 1H), 4.97-4.77 (dd, 1H), 4.38 (q, 1H), 4.28-3.93 (m, 4H), 3.55 (m, 1H), 3.29 (s, 3H), 2.67-2.60, 2.45-2.39 (m, 1H), 2.32-2.25, 2.04-1.97 (m, 1H), 1.10, 0.95 (t, 3H). MS (EI) for C₂₉H₃ᵢFN₂O₅S, found 539 (MH+).

3-Ethyl-4-({7-[4-(5-fluoro-1H-benzimidazol-2-yl)phenyl]-2,3-dihydro-1,4-benzoxazepin-4(5H)-yl}carbonyl)-N-(1-methylethyl)benzenesulfonamide. ¹H NMR (400 MHz, Methanol-d₄): δ 8.16-8.10 (m, 2H), 7.86-7.73 (m, 3H), 7.63-7.56 (m, 4H), 7.35-7.27 (m, 2H), 7.16-7.03 (m, 2H), 6.61 (m, 1H), 4.55-4.10 (m, 5H), 3.37 (m, 1H), 2.68-2.26 (m, 2H), 1.24-1.16 (m, 3H), 1.03-0.98 (m, 6H). MS (EI) for C₃₄H₃₃FN₄O₄S, found 613 (MH+).

4-{[2-Ethyl-3-fluoro-4-(methylsulfonyl)phenyl]carbonyl}-7-[4-(4-methylpiperazin-1-yl)phenyl]-2,3,4,5-tetrahydro-1,4-benzoxazepine. ¹H NMR (400 MHz, DMSO-d₆): δ 7.75-7.68 (m, 1H), 7.51-7.49 (d, 1H), 7.44-7.39 (m, 1H), 7.27-7.13 (m, 2H), 7.01-6.98 (m, 2H), 6.91-6.89 (d, 1H), 7.57, 6.69 (m, 1H), 4.98-4.74 (dd, 1H), 4.35 (q, 1H), 4.31-4.25 (m, 1H), 4.18-3.92 (m, 4H), 3.61-3.52 (m, 1H), 3.17-3.14 (m, 2H), 3.13-3.10 (m, 2H), 2.67-2.48 (m, 2H), 2.33-2.25 (m, 1H), 2.07-1.97 (m, 1H), 1.10, 0.92 (t, 2H). MS (EI) for C₃₀H₃₄FN₃O₄S, found 553 (MH+).

7-[6-(5-Fluoro-1H-benzimidazol-2-yl)pyridin-3-yl]-4-{[3-fluoro-2-methyl-4-(methylsulfonyl)phenyl]carbonyl}-2,3,4,5-tetrahydro-1,4-benzoxazepine. ¹H NMR (400 MHz, DMSO-d₆): δ 13.32-13.18 (m, 1H), 11.99 (bs, 1H), 9.10-8.85 (m, 1H), 8.41-8.27 (m, 1H), 8.0-7.90 (m, 1H), 7.82-7.70 (m, 2H), 7.57-7.49 (m, 1H), 7.33-7.25 (m, 1H), 7.23-7.05 (m, 2H), 6.99 (s, 1H), 5.03-4.92 (m, 1H), 4.60-4.15 (m, 4H), 3.67-3.41 (m, 1H), 3.34 (d, 3H), 2.20-1.75 (m, 3H). MS (EI) for C₃₀H₂₄F₂N₄O₄S, found 575 (MH+).

7-[4-(5-Fluoro-1H-benzimidazol-2-yl)phenyl]-4-{[4-(methylsulfonyl)phenyl]carbonyl}-2,3,4,5-tetrahydro-1,4-benzoxazepine. ¹H NMR (400 MHz, Methanol-d₄): δ 8.16-8.03 (m, 3H), 7.84-7.52 (m, 7.5H), 7.33-7.27 (m, 1H), 7.16-7.05 (m, 2H), 6.80 (m, 0.5H), 4.93 (bs, 1H), 4.57 (bs, 1H), 4.33-4.29 (m, 1H), 4.19-4.12 (m, 2H), 3.85-3.80 (m, 1H), 3.16 (s, 3H). MS (EI) for C₃₀H₂₄FN₃O₄S, found 542 (MH+).

4-{[2-Ethyl-4-(methylsulfonyl)phenyl]carbonyl}-7-[4-(5-fluoro-1H-benzimidazol-2-yl)phenyl]-2,3,4,5-tetrahydro-1,4-benzoxazepine. ¹H NMR (400 MHz, Methanol-d₄): δ 8.17-8.09 (m, 2H), 7.96-7.77 (m, 3H), 7.65-7.58 (m, 3H), 7.43-7.34 (m, 2H), 7.16-7.05 (m, 2H), 4.53-4.13 (m, 4H), 3.68-3.66 (m, 1H), 4.33-4.29 (m, 1H), 4.19-4.12 (m, 1H), 3.85-3.80 (m, 1H), 3.16 (s, 3H), 2.70-2.28 (m, 2H), 1.25-1.08 (m, 3H). MS (EI) for C₃₂H₂₈FN₃O₄S, found 570 (MH+).

4-{[2-Ethyl-3-fluoro-4-(methylsulfonyl)phenyl]carbonyl}-7-(4-piperazin-1-ylphenyl)-2,3,4,5-tetrahydro-1,4-benzoxazepine. ¹H NMR (400 MHz, DMSO-d₆): δ 7.76-7.68 (m, 1H), 7.51-7.49 (d, 1H), 7.45-7.39 (m, 1H), 7.27-7.13 (m, 2H), 7.01-6.96 (m, 2H), 6.90-6.88 (d, 1H), 7.57, 6.61 (m, 1H), 5.00-4.74 (dd, 1H), 4.37 (q, 1H), 4.30-4.23 (m, 1H), 4.18-4.03 (m, 3H), 4.00-3.91 (m, 1H), 3.59-3.52 (m, 2H), 3.34-3.27 (d, 2H), 3.10-3.00 (m, 2H), 2.82 (bs, 3H), 2.64-2.60 (m, 1H), 2.45-2.37 (m, 1H), 2.32-2.23 (m, 1H), 2.05-1.94 (m, 1H), 1.08, 0.92 (t, 3H). MS (EI) for C₂₉H₃₂FN₃O₄S, found 538 (MH+).

4-{[2-Ethyl-4-(ethylsulfonyl)phenyl]carbonyl}-7-[4-(5-fluoro-1H-benzimidazol-2-yl)phenyl]-2,3,4,5-tetrahydro-1,4-benzoxazepine. ¹H NMR (400 MHz, Methanol-d₄): δ 8.16-8.08 (m, 2H), 7.89-7.03 (m, 9.5H), 6.59 (m, 0.5H), 4.54-4.10 (m, 5H), 3.66 (m, 1H), 2.72-2.28 (m, 2H), 1.25-1.06 (m, 6H). MS (EI) for C₃₃H₃₀FN₃O₄S, found 584 (MH+).

7-[4-(5-Fluoro-1H-benzimidazol-2-yl)phenyl]-4-{[2-methyl-4-(methylsulfonyl)phenyl]carbonyl}-2,3,4,5-tetrahydro-1,4-benzoxazepine. ¹H NMR (400 MHz, Methanol-d₄): δ 8.14-8.06 (m, 2H), 7.92-7.74 (m, 3.5H), 7.62-7.52 (m, 3H), 7.43-7.26 (m, 2H), 7.16-7.00 (m, 2H), 5.03 (m, 0.5H), 4.52-4.06 (m, 5H), 3.15 (s, 3H), 2.16 (s, 3H). MS (EI) for C₃₁H₂₆FN₃O₄S, found 556 (MH+).

### Example 2

### [7-(6-Aminopyridin-3-yl)-2,3-dihydro-1,4-benzoxazepin-4(5H)-yl][3-fluoro-2-methyl-4-(methylsulfonyl)phenyl]methanone

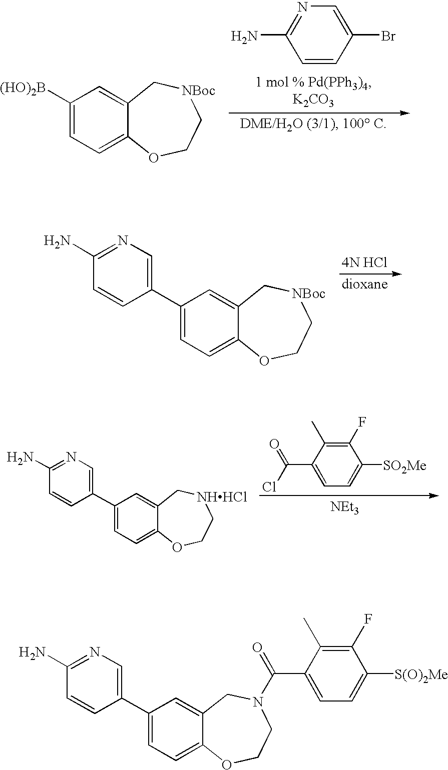

tert-Butyl 7-(6-aminopyridin-3-yl)-2,3-dihydrobenzo[f][1,4]oxazepine-4(5H)-carboxylate. To a mixture of 4-(tert-butoxycarbonyl)-2,3,4,5-tetrahydrobenzo[f][1,4]oxazepin-7-ylboronic acid (1.52 g, 5.2 mmol), prepared as described in Reference Example 5, 2-amino-5-bromopyridine (900 mg, 5.2 mmol), and potassium carbonate (1.73 g, 12.5 mmol) in 1,2-dimethoxyethane/water (30 mL/10 mL) was added tetrakis(triphenylphosphine)palladium(0) (90 mg, 1.5 mol %) and the reaction mixture was purged with nitrogen and stirred at reflux for 3 h. The reaction was cooled to rt, diluted with water/ethyl acetate (50 mL/50 mL), and the separated aqueous layer was extracted with ethyl acetate. The resulting emulsion was removed by filtration. The combined organic layer was washed with brine, dried with sodium sulfate, filtered and concentrated under reduced pressure, and the residue was triturated with toluene for 1 h. The resulting off-white solid was isolated by filtration to give the desired product (1.37 g, 77%) as an off-white solid. MS (EI) for C₁₉H₂₃N₃O₃: 342 (MH⁺).

5-(2,3,4,5-Tetrahydrobenzo[f][1,4]oxazepin-7-yl)pyridine-2-amine. To a stirred solution of tert-butyl 7-(6-aminopyridin-3-yl)-2,3-dihydrobenzo[f][1,4]oxazepine-4(5H)-carboxylate (1.36 g, 3.98 mmol) in 1,4-dioxane (5 mL) was added 4 N hydrogen chloride in 1,4-dioxane (5 mL) and the reaction mixture was stirred at rt overnight. The reaction was concentrated on a rotary evaporator and the residue was triturated with ether. The solid was isolated by filtration. This solid was dissolved in water (5 mL) and made basic with 5 N sodium hydroxide to pH 11-12. The brownish sticky oil that aggregated at the bottom was isolated and the aqueous layer was extracted with 5% methanol in ethyl acetate. The extracts were dried with sodium sulfate and concentrated on a rotary evaporator. The brownish sticky oil was dissolved with a mixture of methanol/ethyl acetate, combined with the isolated organic residue and concentrated under reduced pressure to give a yellow solid. This solid was triturated with dichloromethane (10 mL) for 1 h and a yellow solid was isolated by filtration and dried under high vacuum to give amine the desired product (920 mg, 96%). MS (EI) for C₁₄H₁₅N₃O: 242 (MH⁺).

[7-(6-Aminopyridin-3-yl)-2,3-dihydro-1,4-benzoxazepin-4(5H)-yl][3-fluoro-2-methyl-4-(methylsulfonyl)phenyl]methanone. To a stirred suspension of 5-(2,3,4,5-tetrahydrobenzo[f][1,4]oxazepin-7-yl)pyridine-2-amine (85 mg, 352 μmol) and triethylamine (54 μL, 387 μmol) in dichloromethane (10 mL) was added 3-fluoro-2-methyl-4-(methylsulfonyl)benzoyl chloride (91 mg, in 3 mL of dichloromethane), prepared as described in Reference Example 1, at 0° C. for 2 h. After stirring for an additional 1 h at rt, the reaction mixture was diluted with water (5 mL) and the separated aqueous layer was extracted with dichloromethane. The combined extracts were dried with sodium sulfate, filtered and concentrated under reduced pressure to give a light-yellow solid that was purified via silica gel chromatography to give the desired product (113 mg, 70%) as a white solid. ¹H NMR (400 MHz, DMSO-d₆): δ 8.24-8.03 (dd, 1H), 7.79-7.71 (m, 1H), 7.71-7.69 (dd, 0.5H), 7.57-7.57 (d, 0.5H), 7.44-7.40 (m, 1.5H), 7.29-7.19 (dd, 1H), 7.05-7.01 (dd, 1H), 6.64-6.63 (d, 0.5H), 6.54-6.45 (dd, 1H), 6.06 (s, 2H), 4.93-4.31 (m, 2H), 4.31-3.54 (m, 4H), 3.37-3.36 (d, 3H), 2.12-1.77 (d, 3H). MS (EI) C₂₃H₂₂FN₃O₄S: 456 (MH⁺).

Using the same or analogous synthetic techniques and substituting with appropriate reagents, the following examples were prepared.

7-(1H-Benzimidazol-6-yl)-4-{[4-(methylsulfonyl)phenyl]carbonyl}-2,3,4,5-tetrahydro-1,4-benzoxazepine. ¹H NMR (400 MHz, DMSO-d₆): δ 12.5 (s, 1H), 8.33-6.79 (m, 11H), 4.88 (m, 1H), 4.54 (s, 1H), 4.29 (s, 1H), 4.17 (s, 1H), 4.05 (s, 1H), 3.73 (s, 1H), 3.28 (s, 3H). MS (EI) for C₂₄H₂₁N₃O₄S, found 448 (MH+).

2,2,2-Trifluoro-1-(4-{[7-(1H-indazol-6-yl)-2,3-dihydro-1,4-benzoxazepin-4(5H)-yl]carbonyl}phenyl)ethanol. ¹H NMR (400 MHz, DMSO-d₆): δ 13.17 (s, 1H), 8.18-6.81 (m, 11H), 5.31 (m, 2H), 4.82 (s, 1H), 4.52-3.93 (m, 4H), 3.75 (s, 1H). MS (EI) for C₂₅H₂₀F₃N₃O₃, found 468 (MH+).

7-(1H-Indazol-6-yl)-4-{[4-(1,2,3-thiadiazol-4-yl)phenyl]carbonyl}-2,3,4,5-tetrahydro-1,4-benzoxazepine. ¹H NMR (400 MHz, DMSO-d₆): δ 13.41 (s, 1H), 9.71 (s, 1H), 8.35-7.01 (m, 11H), 4.91 (s, 1H), 4.68-4.15 (m, 4H), 3.82 (s, 1H). MS (EI) for C₂₅H₁₉N₅O₂S, found 454 (MH+).

1,1-Dimethylethyl 4-(4-{[4-(trifluoroacetyl)phenyl]carbonyl}-2,3,4,5-tetrahydro-1,4-benzoxazepin-7-yl)-1H-pyrazole-1-carboxylate. ¹H NMR (400 MHz, DMSO-d₆): δ 8.74 (s, 1H), 8.48-6.68 (m, 8H), 4.86 (d, 2H), 4.47-4.02 (m, 2H), 3.71 (s, 2H), 1.61 (s, 9H). MS (EI) for C₂₆H₂₄F₃N₃O₅, found 516 (MH+).

2,2,2-Trifluoro-1-(4-{[7-(1H-indazol-5-yl)-2,3-dihydro-1,4-benzoxazepin-4(5H)-yl]carbonyl}phenyl)ethanone. ¹H NMR (400 MHz, DMSO-d₆): δ 13.012 (br.s, 1H), 8.13-8.04 (m, 3H), 7.88-7.35 (m, 6H), 7.18-6.70 (m, 2H), 4.96 (s, 1H), 4.52 (s, 1H), 4.28 (s, 1H), 4.15 (s, 1H), 4.02 (s, 1H), 3.70 (s, 1H). MS (EI) for C₂₅H₁₈F₃N₃O₃, found 466 (MH+).

4-{[7-(1H-Indazol-6-yl)-2,3-dihydro-1,4-benzoxazepin-4(5H)-yl]carbonyl}benzenesulfonamide. ¹H NMR (400 MHz, DMSO-d₆): δ 13.2 (s, 1H), 8.17-7.13 (m, 13H), 4.92 (s, 1H), 4.61-4.17 (m, 4H), 3.87 (s, 1H), 3.43 (s, 3H). MS (EI) for C₂₃H₂₀N₄O₄S, found 449 (MH+).

2,2,2-Trifluoro-1-(4-{[7-(1H-indazol-3-yl)-2,3-dihydro-1,4-benzoxazepin-4(5H)-yl]carbonyl}phenyl)ethanone. ¹H NMR (400 MHz, DMSO-d₆): δ 13.2 (s, 1H), 8.17-7.11 (m, 11H), 4.85 (d, 1H), 4.59-4.09 (m, 4H), 3.87 (s, 1H), 3.82 (s, 1H). MS (EI) for C₂₅H₁₈F₃N₃O₃, found 466 (MH+).

4-{[7-(1H-Indazol-3-yl)-2,3-dihydro-1,4-benzoxazepin-4(5H)-yl]carbonyl}benzenesulfonamide. ¹H NMR (400 MHz, DMSO-d₆): δ 13.2 (s, 1H), 8.11-7.13 (m, 13H), 4.92 (s, 1H), 4.61-4.03 (m, 4H), 3.76 (s, 1H). MS (EI) for C₂₃H₂₀N₄O₄S, found 449 (MH+).

4-{[7-(1H-Benzimidazol-5-yl)-2,3-dihydro-1,4-benzoxazepin-4(5H)-yl]carbonyl}benzenesulfonamide. ¹H NMR (400 MHz, DMSO-d₆): δ 8.27 (s, 1H), 7.93-7.21 (m, 10H), 7.11 (m, 2H), 4.89 (s, 1H), 4.55 (s, 1H), 4.31 (s, 1H), 4.17 (s, 1H), 3.74 (s, 1H). MS (EI) for C₂₃H₂₀N₄O₄S, found 449 (MH+).

1-(4-{[7-(1H-Benzimidazol-6-yl)-2,3-dihydro-1,4-benzoxazepin-4(5H)-yl]carbonyl}phenyl)-2,2,2-trifluoroethanone. ¹H NMR (400 MHz, DMSO-d₆): δ 11.4 (s, 1H), 8.27-6.73 (m, 11H), 4.87 (s, 1H), 4.57-4.17 (m, 4H), 3.78 (s, 1H). MS (EI) for C₂₅H₁₈F₃N₃O₃, found 466 (MH+).

4-{[4-(Methylsulfonyl)phenyl]carbonyl}-7-(1H-pyrazolo[3,4-b]pyridin-6-yl)-2,3,4,5-tetrahydro-1,4-benzoxazepine. ¹H NMR (400 MHz, DMSO-d₆): δ 13.63 (s, 1H), 8.83-8.49 (m, 2H), 8.24-7.96 (m, 2H), 7.79 (s, 1H), 7.68-7.61 (m, 2H), 7.50 (m, 1H), 7.13 (m, 1H), 7.81 (s, 1H), 6.91 (s, 1H), 4.89 (s, 1H), 4.57 (m, 1H), 4.31 (s, 1H), 4.18 (s, 1H), 4.06 (s, 1H), 3.72 (s, 3H), 3.64 (s, 3H). MS (EI) for C₂₃H₂₀N₄O₄S, found 449 (MH+).

4-{[7-(1H-Pyrazolo[3,4-b]pyridin-6-yl)-2,3-dihydro-1,4-benzoxazepin-4(5H)-yl]carbonyl}benzenesulfonamide. ¹H NMR (400 MHz, DMSO-d₆): δ 13.72 (s, 1H), 8.85-8.47 (m, 2H), 8.21 (m, 1H), 7.94-7.75 (m, 2H), 7.63-7.58 (m, 2H), 7.42-7.56 (m, 3H), 7.15-6.93 (m, 2H), 4.89 (s, 1H), 4.55 (s, 1H), 4.32 (s, 1H), 4.18 (s, 1H), 4.06 (s, 1H), 3.76 (s, 1H). MS (EI) for C₂₂H₁₉N₅O₄S, found 450 (MH+).

2,2,2-Trifluoro-1-(4-{[7-(1H-pyrazolo[3,4-b]pyridin-3-yl)-2,3-dihydro-1,4-benzoxazepin-4(5H)-yl]carbonyl}phenyl)ethanone. ¹H NMR (400 MHz, DMSO-d₆): δ 8.65-7.62 (m, 6H), 7.44-7.11 (m, 4H), 4.72 (s, 1H), 4.63 (s, 1H), 4.34 (s, 1H), 4.20 (s, 1H), 4.07 (s, 1H), 3.75 (s, 1H). MS (EI) for C₂₄H₁₇F₃N₄O₃, found 467 (MH+).

7-(1,2-Benzisoxazol-5-yl)-4-{[4-(methylsulfonyl)phenyl]carbonyl}-2,3,4,5-tetrahydro-1,4-benzoxazepine. ¹H NMR (400 MHz, DMSO-d₆): 6) 8.06-7.74 (m, 4H), 7.71-7.44 (m, 4H), 7.14-6.84 (m, 3H), 4.88 (s, 1H), 4.50 (s, 1H), 4.28 (s, 1H), 4.14 (s, 1H), 4.08 (s, 1H), 3.69 (s, 1H, 3.64 (s, 3H). MS (EI) for C₂₄H₂₀N₂O₅S, found 449 (MH+).

4-{[7-(1,2-Benzisoxazol-5-yl)-2,3-dihydro-1,4-benzoxazepin-4(5H)-yl]carbonyl}benzenesulfonamide. ¹H NMR (400 MHz, DMSO-d₆): δ 7.95-7.68 (m, 4H), 7.62-7.39 (m, 6H), 6.82-7.12 (m, 3H), 4.85 (s, 1H), 4.51 (s, 1H), 4.28 (s, 1H), 4.15 (s, 1H), 4.08 (s, 1H), 3.72 (s, 1H). MS (EI) for C₂₃H₁₉N₃O₅S, found 450 (MH+).

1-(4-{[7-(1,2-Benzisoxazol-5-yl)-2,3-dihydro-1,4-benzoxazepin-4(5H)-yl]carbonyl}phenyl)-2,2,2-trifluoroethanone. ¹H NMR (400 MHz, DMSO-d₆): δ 8.12-7.78 (m, 2H), 7.75-7.62 (m, 5H), 7.55-7.38 (m, 2H), 7.27-6.70 (m, 2H), 4.85 (s, 1H), 4.47 (s, 1H), 4.25 (s, 1H), 4.15 (s, 1H), 4.02 (s, 1H), 3.73 (s, 1H). MS (EI) for C₂₅H₁₇F₃N₂O₄, found 467 (MH+).

4-{[4-(Methylsulfonyl)phenyl]carbonyl}-7-(1,8-naphthyridin-3-yl)-2,3,4,5-tetrahydro-1,4-benzoxazepine. ¹H NMR (400 MHz, DMSO-d₆): δ 9.48-7.17 (m, 12H), 4.94 (s, 1H), 4.59 (s, 1H), 4.39 (s, 1H), 4.08 (s, 1H), 3.76 (s, 1H), 3.27 (s, 3H). MS (EI) for C₂₅H₂₁N₃O₄S, found 460 (MH+).

2,2,2-Trifluoro-1-(4-{[7-(1,8-naphthyridin-3-yl)-2,3-dihydro-1,4-benzoxazepin-4(5H)-yl]carbonyl}phenyl)ethanone. ¹H NMR (400 MHz, DMSO-d₆): δ 9.53-6.97 (m, 12H), 4.93 (s, 1H), 4.58 (s, 1H), 4.38 (s, 1H), 4.11 (s, 1H), 3.80 (s, 1H), 3.26 (s, 3H). MS (EI) for C₂₆H₁₈F₃N₃O₃, found 478 (MH+).

2-Methyl-5-(4-{[4-(methylsulfonyl)phenyl]carbonyl}-2,3,4,5-tetrahydro-1,4-benzoxazepin-7-yl)aniline. ¹H NMR (400 MHz, d₆-DMSO): δ 8.01 (d, 1H), 7.97 (d, 2H), 7.64 (d, 2H), 7.71-7.38 (m, 2H), 7.09-6.53 (m, 3H), 4.91 (s, 1H), 4.86 (s, 2H), 4.43 (s, 1H), 4.25 (s, 1H), 4.17 (s, 1H), 4.06 (s, 1H), 3.71 (s, 1H), 3.27 (s, 3H), 2.03 (s, 3H). MS (EI) for C₂₄H₂₄N₂O₄S, found 437 (MH+).

1-(4-{[7-(3-Amino-4-methylphenyl)-2,3-dihydro-1,4-benzoxazepin-4(5H)-yl]-carbonyl}phenyl)-2,2,2-trifluoroethanone. ¹H NMR (400 MHz, d₆-DMSO): δ 8.21-6.49 (m, 10H), 4.89 (s, 2H), 4.81 (s, 1H), 4.48 (s, 1H), 4.27 (s, 1H), 4.17 (s, 1H), 4.07 (s, 1H), 3.72 (s, 1H), 2.14 (s, 3H). MS (EI) for C₂₅H₂ᵢF₃N₂O₃, found 455 (MH+).

7-(1,3-Benzothiazol-6-yl)-4-{[4-(methylsulfonyl)-phenyl]carbonyl}-2,3,4,5-tetrahydro-1,4-benzoxazepine. ¹H NMR (400 MHz, DMSO-d₆): δ 9.4 (s, 1H), 8.54-6.94 (m, 10H), 4.91 (s, 1H), 4.58-4.03 (m, 4H), 3.75 (s, 1H), 3.27 (s, 3H). MS (EI) for C₂₄H₂₀N₂O₄S₂, found 465 (MH+).

2,2,2-Trifluoro-1-[4-({7-[4-(1H-imidazol-2-yl)phenyl]-2,3-dihydro-1,4-benzoxazepin-4(5H)-yl}carbonyl)phenyl]ethane-1,1-diol. ¹H NMR (400 MHz, DMSO-d₆): δ 8.39 (m, 1H), 8.01 (d, 2H), 7.89-7.47 (m, 8H), 7.18-6.80 (m, 2H), 4.84 (s, 1H), 4.57 (s, 1H), 4.30 (s, 1H), 4.18 (s, 1H), 4.06 (s, 1H), 3.68 (s, 1H). MS (EI) for C₂₇H₂₂F₃N₃O₄, found 510 (MH+).

7-(2,4-Dimethyl-1H-benzimidazol-6-yl)-4-{[4-(methylsulfonyl)-phenyl]carbonyl}-2,3,4,5-tetrahydro-1,4-benzoxazepine. ¹H NMR (400 MHz, DMSO-d₆): δ 8.85-7.96 (m, 2H), 7.60-7.69 (m, 2H), 7.55-7.18 (m, 3H), 7.09-6.87 (m, 2H) 4.87 (s, 1H), 4.51 (s, 1H), 4.38 (s, 1H), 4.14 (s, 1H), 4.07 (s, 1H), 3.72 (s, 1H), 3.35 (s, 3H), 2.51 (s, 3H), 1.91 (s, 3H). MS (EI) for C₂₆H₂₅N₃O₄S, found 476 (MH+).

4-{[4-(Methylsulfonyl)phenyl]carbonyl}-7-(2,4,5-trimethyl-1H-benzimidazol-6-yl)-2,3,4,5-tetrahydro-1,4-benzoxazepine. ¹H NMR (400 MHz, DMSO-d₆): δ 8.0-7.93 (m, 2H), 7.67 (d, 1H), 7.53 (d, 1H), 7.28 (s, 1H), 7.14 (t, 1H), 7.04 (m, 1H), 4.82 (s, 1H), 4.51 (s, 1H), 4.31 (s, 1H), 4.18 (s, 1H), 4.07 (s, 1H), 3.72 (s, 1H), 3.35 (s, 3H), 2.51 (s, 3H), 2.18 9s, 3H), 2.07 (s, 3H). MS (EI) for C₂₇H₂₇N₃O₄S, found 490 (MH+).

7-(2,4-Dimethyl-1H-benzimidazol-6-yl)-4-{[4-(1-methylethyl)-phenyl]carbonyl}-2,3,4,5-tetrahydro-1,4-benzoxazepine. ¹H NMR (400 MHz, DMSO-d₆): δ 7.63-7.45 (m, 2H), 7.35-7.15 (m, 4H), 6.78-7.05 (m, 3H), 4.82 (s, 1H), 4.52 (s, 1H), 4.23 (s, 1H), 4.18 (s, 1H), 3.92 (s, 1H), 3.77 (s, 1H), 2.87 (m, 1H), 2.58 (s, 3H), 2.52 (s, 3H), 1.31 (s, 6H). MS (EI) for C₂₈H₂₀N₃O₂, found 440 (MH+).

7-(2-Methyl-1H-benzimidazol-5-yl)-4-{[4-(methylthio)-phenyl]carbonyl}-2,3,4,5-tetrahydro-1,4-benzoxazepine. ¹H NMR (400 MHz, DMSO-d₆): δ 7.65 (s, 1H), 7.49 (d, 2H), 7.39-7.28 (m, 6H), 7.04 (m, 1H), 4.82 (s, 1H), 4.57 (s, 1H), 4.25 (s, 1H), 4.14 (s, 1H), 3.98 (s, 1H), 2.50 (s, 6H). MS (EI) for C₂₅H₂₃N₃O₂S found 430.5 (MH+).

4-{[3-Fluoro-4-(trifluoromethyl)phenyl]carbonyl}-7-(2-methyl-1H-benzimidazol-5-yl)-2,3,4,5-tetrahydro-1,4-benzoxazepine. ¹H NMR (400 MHz, DMSO-d₆): δ 7.86 (m, 1 h), 7.67-7.51 (m, 2H), 7.45-7.35 (m, 3H), 7.32-6.97 (m, 3H), 4.86 (s, 1H), 4.55 (s, 1H), 4.22 (s, 1H), 4.15 (s, 1H), 4.08 (s, 1H), 3.73 (s, 1H), 2.50 (s, 3H). MS (EI) for C₂₅H₁₉F₄N₃O₂ found 470.4 (MH+).

(4-(1H-imidazol-1-yl)phenyl)(7-(2-methyl-1H-benzo[d]imidazol-5-yl)-2,3-dihydrobenzo[f][1,4]oxazepin-4(5H)-yl)methanone. ¹H NMR (400 MHz, DMSO-d₆); δ 12.24 (S, 1H), 8.45-8.32 (m, 1H), 7.94-6.90 (m, 12H), 4.87 (s, 1H), 4.72-4.53 (m, 1H), 4.35-3.75 (m, 4H). MS (EI) for C₂₇H₂₃N₅O₂, found 450.2 (MH+).

7-(2-Methyl-1H-benzimidazol-6-yl)-4-{[4-(methylsulfonyl)phenyl]carbonyl}-2,3,4,5-tetrahydro-1,4-benzoxazepin-9-amine. ¹H NMR (400 MHz, DMSO-d₆): δ 12.2 (s, 1H), 8.02-6.81 (m, 9H), 5.95 (d, 2H), 5.02-3.67 (m, 6H), 3.25 (s, 3H), 3.35 (s, 3H). MS (EI) for C₂₅H₂₄N₄O₄S, found 477 (MH+).

4-({4-[(2-Chlorophenyl)sulfonyl]-2-methylphenyl}carbonyl)-7-(2-methyl-1H-benzimidazol-6-yl)-2,3,4,5-tetrahydro-1,4-benzoxazepine. ¹H NMR (400 MHz, DMSO-d₆): δ 12.2 (s, 1H), 8.35 (s, 1H), 7.80-6.6 (m, 12H), 4.95 (d, 1H), 4.31-4.02 (m, 4H), 3.51 (s, 3H), 3.38 (s, 3H), 2.63 (s, 3H). MS (EI) for C₃₁H₂₆ClN₃O₄S, found 473 (MH+).

4-({4-[(3-Chlorophenyl)sulfonyl]-2-methylphenyl}carbonyl)-7-(2-methyl-1H-benzimidazol-6-yl)-2,3,4,5-tetrahydro-1,4-benzoxazepine. ¹H NMR (400 MHz, DMSO-d₆): δ 12.2 (s, 1H), 8.11-6.58 (m, 13H), 4.96 (d, 1H), 4.38-4.01 (m, 4H), 3.52 (s, 1H), 3.37 (s, 3H), 2.63 (s, 3H). MS (EI) for C₃₁H₂₆ClN₃O₄S, found 473 (MH+).

4-({4-[(4-Chlorophenyl)sulfonyl]-2-methylphenyl}carbonyl)-7-(2-methyl-1H-benzimidazol-6-yl)-2,3,4,5-tetrahydro-1,4-benzoxazepine. ¹H NMR (400 MHz, DMSO-d₆): δ 12.2 (s, 1H), 8.07-6.59 (m, 13H), 4.90 (d, 1H), 4.40-4.08 (m, 4H), 3.53 (s, 1H), 3.39 (s, 3H), 2.62 (s, 3H). MS (EI) for C₃₁H₂₆ClN₃O₄S, found 473 (MH+).

4-{[2-Ethyl-4-(methylsulfonyl)phenyl]carbonyl}-7-(5-fluoro-2-methyl-1H-benzimidazol-6-yl)-2,3,4,5-tetrahydro-1,4-benzoxazepine. ¹H NMR (400 MHz, DMSO-d₆): δ 7.87-6.60 (m, 8H), 4.90 (m, 1H), 4.40-4.07 (m, 4H), 3.25 (s, 1H), 3.2 (s, 3H), 2.61 (s, 3H), 2.73-2.41 (m, 2H), 1.18 and 1.08 (t, 3H). MS (EI) for C₂₇H₂₆FN₃O₄S, found 508 (MH+).

4-{[2-Ethyl-4-(methylsulfonyl)phenyl]carbonyl}-7-(4-fluoro-2-methyl-1H-benzimidazol-6-yl)-2,3,4,5-tetrahydro-1,4-benzoxazepine. ¹H NMR (400 MHz, DMSO-d₆): δ 8.02-6.73 (m, 8H), 4.91 (m, 1H), 4.43-4.01 (m, 4H), 3.60 (s, 1H), 3.32 (s, 3H), 2.61 (s, 3H), 2.65-2.32 (m, 2H), 1.17 and 1.09 (t, 3H). MS (EI) for C₂₇H₂₆FN₃O₄S, found 508 (MH+).

4-{[2,3-Difluoro-4-(methylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 12.2 (m, 1H), 7.83-7.01 (m, 8H), 4.87 (s, 1H), 4.58-4.02 (m, 4H), 3.72 (s, 1H), 3.42 (s, 3H), 2.61 (s, 3H). MS (EI) for C₂₅H₂₁F₂N₃O₄S, found 498 (MH+).

4-{[3-Fluoro-2-methyl-4-(methylsulfonyl)phenyl]carbonyl}-7-(1H-pyrazol-4-yl)-2,3,4,5-tetrahydro-1,4-benzoxazepine. ¹H NMR (400 MHz, DMSO-d₆): δ 7.93-6.67 (m, 7H), 4.82 (m, 1H), 4.44-3.91 (m, 4H), 3.62 (m, 1H), 3.32 (s, 3H), 2.11 and 1.77 (s, 3H). MS (EI) for C₂₁H₂₀FN₃O₄S, found 430 (MH+).

4-{[2-Ethyl-4-(methylsulfonyl)phenyl]carbonyl}-7-(1H-pyrazol-4-yl)-2,3,4,5-tetrahydro-1,4-benzoxazepine. ¹H NMR (400 MHz, DMSO-d₆): δ 12.9 (s, 1H), 8.23-6.68 (m, 7H), 5.84 (m, 1H), 4.44-4.02 (m, 4H), 3.52 (s, 1H), 3.31 (s, 3H), 2.64-2.36 (m, 2H), 1.21 and 1.02 (t, 3H). MS (EI) for C₂₂H₂₃N₃O₄S, found 426 (MH+).

4-{[3-Fluoro-2-methyl-4-(methylsulfonyl)phenyl]carbonyl}-7-(1H-indazol-6-yl)-2,3,4,5-tetrahydro-1,4-benzoxazepine. ¹H NMR (400 MHz, DMSO-d₆): δ 13.1 (d, 1H), 8.07-6.79 (m, 9H), 4.9 (m, 1H), 4.61-3.93 (m, 4H), 3.59 (m, 1H), 3.31 (s, 3H), 2.1 and 1.8 (s, 3H). MS (EI) for C₂₅H₂₂FN₃O₄S, found 480 (MH+).

4-{[2-Ethyl-3-fluoro-4-(methylsulfonyl)phenyl]carbonyl}-7-(1H-indazol-6-yl)-2,3,4,5-tetrahydro-1,4-benzoxazepine. ¹H NMR (400 MHz, DMSO-d₆): δ 13.1 (d, 1H), 8.09-6.62 (m, 9H), 4.91 (m, 1H), 4.49-4.02 (m, 4H), 3.62 (s, 1H), 3.40 (s, 3H), 2.71-2.13 (m, 2H), 1.18 and 1.02 (t, 3H). MS (EI) for C₂₆H₂₄FN₃O₄S, found 494 (MH+).

4-{[3-Fluoro-2-methyl-4-(methylsulfonyl)phenyl]carbonyl}-7-(1H-indazol-5-yl)-2,3,4,5-tetrahydro-1,4-benzoxazepine. ¹H NMR (400 MHz, DMSO-d₆): δ 13.0 (d, 1H), 8.13-6.70 (m, 9H), 4.91 (m, 1H), 4.57-3.97 (m, 4H), 3.60 (s, 1H), 3.40 (s, 3H), 2.18 and 1.8 (s, 3H). MS (EI) for C₂₅H₂₂FN₃O₄S, found 480 (MH+).

4-{[2-Ethyl-3-fluoro-4-(methylsulfonyl)phenyl]carbonyl}-7-(1H-indazol-5-yl)-2,3,4,5-tetrahydro-1,4-benzoxazepine. ¹H NMR (400 MHz, DMSO-d₆): δ 13.1 (d, 1H), 8.11-6.78 (m, 9H), 4.92 (m, 1H), 4.42-3.63 (m, 5H), 2.64-2.13 (m, 2H), 2.46 (s, 3H), 1.18 and 1.03 (t, 3H). MS (EI) for C₂₆H₂₄FN₃O₄S, found 494 (MH+).

N-[4-(4-{[2-Ethyl-3-fluoro-4-(methylsulfonyl)-phenyl]carbonyl}-2,3,4,5-tetrahydro-1,4-benzoxazepin-7-yl)-2-methylphenyl]acetamide. ¹H NMR (400 MHz, DMSO-d₆): δ 9.37 (s, 1H), 7.76-6.73 (m, 8H), 4.92 (m, 1H), 4.41-4.03 (m, 4H), 3.60 (s, 1H), 3.4 (s, 3H), 3.63-2.32 (m, 2H), 2.6 (s, 3H), 2.24 (s, 3H), 1.18 and 1.01 (t, 3H). MS (EI) for C₂₈H₂₉FN₂O₅S, found 525 (MH+).

N-[2-Ethyl-4-(4-{[2-ethyl-3-fluoro-4-(methylsulfonyl)-phenyl]carbonyl}-2,3,4,5-tetrahydro-1,4-benzoxazepin-7-yl)phenyl]acetamide. ¹H NMR (400 MHz, DMSO-d₆): δ 9.34 (d, 1H), 7.80-6.78 (m, 8H), 4.91 (m, 1H), 4.41-4.02 (m, 4H), 3.62 (s, 1H), 3.38 (s, 3H), 2.76-2.12 (m, 4H), 2.12 (s, 3H), 1.18-098 (m, 6H). MS (EI) for C₂₉H₃₁FN₂O₅S, found 539 (MH+).

4-(4-{[2-Ethyl-3-fluoro-4-(methylsulfonyl)phenyl]carbonyl}-2,3,4,5-tetrahydro-1,4-benzoxazepin-7-yl)-2-fluoroaniline. ¹H NMR (400 MHz, DMSO-d₆): δ 9.87 (d, 1H), 8.0-6.72 (m, 8H), 4.92 (m, 1H), 4.61-4.01 (m, 4H), 3.61 (s, 1H), 3.39 (s, 3H), 2.68-2.13 (m, 2H), 1.18 and 0.98 (t, 3H). MS (EI) for C₂₅H₂₄F₂N₂O₄S, found 487 (MH+).

2,6-Dichloro-4-(4-{[2-ethyl-3-fluoro-4-(methylsulfonyl)-phenyl]carbonyl}-2,3,4,5-tetrahydro-1,4-benzoxazepin-7-yl)aniline. ¹H NMR (400 MHz, DMSO-d₆): δ 7.72 (m, 1H), 7.58 (m, 1H), 7.45-7.28 (m, 2H), 7.13-6.72 (m, 3H), 5.7 (d, 2H), 5.01-4.72 (m, 1H), 4.38 (s, 1H), 4.32-4.03 (m, 3H), 3.54 (m, 1H), 3.34 (s, 3H), 2.7-2.08 9m, 2H), 1.09 and 10.98 (t, 3H). MS (EI) for C₂₅H₂₃Cl₂FN₂S₄, found 538 (MH+).

4-(4-{[2-Ethyl-3-fluoro-4-(methylsulfonyl)phenyl]carbonyl}-2,3,4,5-tetrahydro-1,4-benzoxazepin-7-yl)-2-(trifluoromethyl)aniline. ¹H NMR (400 MHz, DMSO-d₆): δ 7.73 (m, 1H), 7.6-6.72 (m, 7H), 5.32 (s, 2H), 4.91 (m, 1H), 4.22-4.01 (m, 4H), 3.59 (s, 1H), 3.35 (s, 3H), 2.64-2.18 (m, 2H), 1.15 and 0.96 (t, 3H). MS (EI) for C₂₅H₂₄F₄N₂O₄S, found 537 (MH+).

N-[4-(4-{[2-Ethyl-3-fluoro-4-(methylsulfonyl)-phenyl]carbonyl}-2,3,4,5-tetrahydro-1,4-benzoxazepin-7-yl)-2-fluorophenyl]acetamide. ¹H NMR (400 MHz, DMSO-d₆): δ 9.2 (d, 1H), 7.74 (m, 1H), 7.58-7.08 (m, 3H), 7.02-6.63 (m, 4H), 5.08-4.83 (d.d, 1H), 4.34 (m, 1H), 4.37-4.01 (m, 3H), 3.60 (m, 1H), 3.35 (s, 3H), 2.17-2.72 (m, 2H), 2.12 (m, 3H), 1.08 and 1.01 (t, 3H). MS (EI) for C₂₇H₂₆F₂N₂O₅S, found 529 (MH+).

N-[4-(4-{[2-Ethyl-3-fluoro-4-(methylsulfonyl)-phenyl]carbonyl}-2,3,4,5-tetrahydro-1,4-benzoxazepin-7-yl)-2-fluorophenyl]methane-sulfonamide. ¹H NMR (400 MHz, DMSO-d₆): δ 9.64 (s, 1H), 7.75-6.80 (m, 8H), 4.97 (m, 1H), 4.51-4.02 (m, 4H), 3.60 (m, 1H), 3.40 (s, 3H), 3.12 (d, 3H), 2.64-2.13 (m, 2H), 1.12 and 1.02 (t, 3H). MS (EI) for C₂₆H₂₆F₂N₂O₆S₂, found 565 (MH+).

4-(4-{[2-Ethyl-3-fluoro-4-(methylsulfonyl)phenyl]carbonyl}-2,3,4,5-tetrahydro-1,4-benzoxazepin-7-yl)-2-methylaniline. ¹H NMR (400 MHz, DMSO-d₆): δ 7.81-6.62 (m, 8H), 4.98 (m, 3H), 4.38-4.0 (m, 4H), 3.60 (m, 1H), 3.36 (s, 3H), 3.12 (d, 3H), 2.66-2.11 (m, 2H), 2.01 (d, 3H), 1.13 and 0.96 (t, 3H). MS (EI) for C₂₆H₂₇FN₂O₄S, found 483 (MH+).

2-Ethyl-4-(4-{[2-ethyl-3-fluoro-4-(methylsulfonyl)-phenyl]carbonyl}-2,3,4,5-tetrahydro-1,4-benzoxazepin-7-yl)aniline. ¹H NMR (400 MHz, DMSO-d₆): δ 7.91-6.40 (m, 8H), 5.11-4.63 (m, 3H), 4.43-3.86 (m, 4H), 3.50 (s, 1H), 3.20 (s, 3H), 2.80-2.0 (m, 4H), 1.12-0.98 (m, 6H). MS (EI) for C₂₇H₂₉FN₂O₄S, found 497 (MH+).

2-Amino-5-(4-{[2-ethyl-3-fluoro-4-(methylsulfonyl)-phenyl]carbonyl}-2,3,4,5-tetrahydro-1,4-benzoxazepin-7-yl)phenol. ¹H NMR (400 MHz, DMSO-d₆): δ 9.09 (d, 1H), 7.79 (m, 1H), 7.44-6.58 (m, 7H), 4.84 (m, 1H), 4.65 (s, 2H), 4.42-3.93 (m, 4H), 3.64 (s, 1H), 3.35 (s, 3H), 2.70-2.0 (m, 2H), 1.12 and 1.0 (t, 3H). MS (EI) for C₂₅H₂₅FN₂O₅S, found 485 (MH+).

N-[4-(4-{[2-Ethyl-3-fluoro-4-(methylsulfonyl)-phenyl]carbonyl}-2,3,4,5-tetrahydro-1,4-benzoxazepin-7-yl)-2-(methyloxy)phenyl]-acetamide. ¹H NMR (400 MHz, DMSO-d₆): δ 9.2 (d, 1H), 8.07-6.84 (m, 8H), 4.94 (m, 1H), 4.44-4.0 (m, 4H), 3.93 (d, 3H), 3.6 (s, 1H), 3.31 (s, 3H), 2.70-2.2 (m, 2H), 2.12 (d, 3H), 1.18 and 1.1 (t, 3H). MS (EI) for C₂₈H₂₉FN₂O₆S, found 541 (MH+).

4-(4-{[2-Ethyl-3-fluoro-4-(methylsulfonyl)phenyl]carbonyl}-2,3,4,5-tetrahydro-1,4-benzoxazepin-7-yl)-2-(methyloxy)aniline. ¹H NMR (400 MHz, DMSO-d₆): δ 7.78 (m, 1H), 7.6-6.6 (m, 7H), 4.89 (m, 1H), 4.48 (d, 2H), 4.41 (s, 1H), 4.21-3.99 (m, 4H), 3.84 (d, 3H), 3.62 (s, 1H), 3.38 (s, 3H), 2.65-2.20 (m, 2H), 1.18 and 1.01 (t, 3H). MS (EI) for C₂₆H₂₇FN₂O₅S, found 499 (MH+).

4-{[2-Ethyl-3-fluoro-4-(methylsulfonyl)phenyl]carbonyl}-7-(3-methyl-1H-indol-5-yl)-2,3,4,5-tetrahydro-1,4-benzoxazepine. ¹H NMR (400 MHz, DMSO-d₆): δ 10.7 (d, 1H), 7.84-6.81 (m, 9H), 4.91 (m, 1H), 4.41 (s, 1H), 4.17-4.03 (m, 3H), 3.62 (s, 1H), 3.4 (s, 3H), 3.38 (s, 3H), 2.5 (m, 2H), 2.25 (d, 3H), 1.08 and 1.05 (t, 3H). MS (EI) for C₂₈H₂₇FN₂O₄S, found 507 (MH+).

N′-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-N,N-dimethylpropane-1,3-diamine. ¹H NMR (400 MHz, DMSO-d₆): δ 8.39-6.47 (m, 8H), 4.82 (m, 1H), 4.47-3.91 (m, 5H), 3.61 (m, 2H), 3.58 (s, 3H), 2.83 (m, 2H), 2.71-1.98 (m, 2H), 2.47 (s, 6H), 1.82 (m, 2H), 1.17 and 0.98 (t, 3H). MS (EI) for C₂₉H₃₅FN₄O₄S, found 555 (MH+).

N′-[5-(4-{[2-ethyl-3-fluoro-4-(methylsulfonyl)-phenyl]carbonyl}-2,3,4,5-tetrahydro-1,4-benzoxazepin-7-yl)pyridin-2-yl]-N,N-dimethyl-ethane-1,2-diamine. ¹H NMR (400 MHz, DMSO-d₆): δ 8.37-6.52 (m, 8H), 4.80 (m, 1H), 4.47-3.91 (m, 5H), 3.61 (m, 2H), 3.50 (m, 2H), 3.57 (s, 3H), 2.84 (m, 2H), 2.69-1.99 (m, 2H), 2.45 (s, 6H), 1.82 (m, 2H), 1.18 and 0.97 (t, 3H). MS (EI) for C₂₈H₃₃FN₄O₄S, found 541 (MH+).

N-[5-(4-{[2-Ethyl-3-fluoro-4-(methylsulfonyl)phenyl]carbonyl}-2,3,4,5-tetrahydro-1,4-benzoxazepin-7-yl)-4-methyl-1,3-thiazol-2-yl]acetamide. ¹H NMR (400 MHz, DMSO-d₆): δ 9.2 (d, 1H), 7.68 (m, 1H), 7.32-7.25 (m, 1H), 7.18-7.12 (m, 2H), 7.03-6.97 (m, 1H), 4.71-4.95 (d.d, 1H), 4.43 (s, 1H), 4.37-4.0 (m, 3H), 3.55 (m, 1H), 3.31 (s, 3H), 2.68-2.11 (m, 2H), 2.49 (s, 3H), 2.01 and 2.22 (s, 3H), 1.08 and 0.98 (t, 3H). MS (EI) for C₂₅H₂₆FN₃O₅S₂, found 532 (MH+).

5-(4-{[2-Ethyl-3-fluoro-4-(methylsulfonyl)phenyl]carbonyl}-2,3,4,5-tetrahydro-1,4-benzoxazepin-7-yl)-4-methyl-1,3-thiazol-2-amine. ¹H NMR (400 MHz, DMSO-d₆): δ 7.69-6.53 (m, 5H), 4.84 (m, 1H), 4.39-3.97 (m, 4H), 3.56 (s, 1H), 3.31 (s, 3H), 2.67-2.15 (m, 2H), 2.09 (d, 3H), 1.18 and 1.01 (t, 3H). MS (EI) for C₂₃H₂₄FN₃O₄S₂, found 490 (MH+).

5-{4-[(4-Bromophenyl)carbonyl]-2,3,4,5-tetrahydro-1,4-benzoxazepin-7-yl}pyridin-2-amine. ¹H NMR (400 MHz, DMSO-d₆): δ 8.09 (s, 1H), 7.64-6.51 (m, 9H), 6.07 (s, 2H), 4.65 (s, 2H), 4.17 (s, 2H), 3.86 (s, 2H). MS (EI) for C₂₁H₁₈BrN₃O₂, found 425.3 (MH+).

5-{4-[(4-Chlorophenyl)carbonyl]-2,3,4,5-tetrahydro-1,4-benzoxazepin-7-yl}pyridin-2-amine. ¹H NMR (400 MHz, DMSO-d₆): δ 8.09 (s, 1H), 7.68-6.51 (m, 9H), 6.05 (s, 2H), 4.65 (s, 2H), 4.17 (s, 2H), 3.86 (s, 2H). MS (EI) for C₂₁H₁₈ClN₃O₂ found 380.8 (MH+).

5-[4-(Biphenyl-4-ylcarbonyl)-2,3,4,5-tetrahydro-1,4-benzoxazepin-7-yl]pyridin-2-amine. ¹H NMR (400 MHz, DMSO-d₆): δ 8.15 (s, 1H), 7.74-7.70 (m, 4H), 7.56-7.35 (m, 8H), 7.02-6.45 (m, 2H), 6.04 (s, 2H), 4.70 (s, 2H), 4.20 (s, 2H), 3.92 (2s, 1H). MS (EI) for C₂₇H₂₃N₃O₂ found 422.5 (MH+).

5-{4-[(1-Methyl-1H-indol-2-yl)carbonyl]-2,3,4,5-tetrahydro-1,4-benzoxazepin-7-yl}pyridin-2-amine. ¹H NMR (400 MHz, DMSO-d₆): δ 8.23-7.72 (m, 1H), 7.60-6.77 (m, 8H), 6.67-6.43 (m, 2H), 6.11 (s, 2H), 4.81 (bs, 2H), 4.22 (bs, 2H), 4.04 (bs, 2H), 3.60 (bs, 3H). MS (EI) for C₂₄H₂₂N₄O₂ found 399.5 (MH+).

5-{4-[(4-Chloro-2-methylphenyl)carbonyl]-2,3,4,5-tetrahydro-1,4-benzoxazepin-7-yl}pyridin-2-amine. ¹H NMR (400 MHz, DMSO-d₆): δ 8.11 (d, 1H), 7.70-7.54 (m, 1H), 7.46-7.16 (m, 4H), 7.08-6.49 (m, 3H), 6.08 (s, 2H), 4.83 (bs, 1H), 4.37 (s, 1H), 4.21-3.01 (m, 3H), 3.53 (bs, 1H), 1.99 (s, 3H). MS (EI) for C₂₂H₂₀ClN₃O₂ found 394.9 (MH+).

5-(4-{[4-(1H-Imidazol-1-yl)phenyl]carbonyl}-2,3,4,5-tetrahydro-1,4-benzoxazepin-7-yl)pyridin-2-amine. ¹H NMR (400 MHz, DMSO-d₆): δ 8.36 (bs, 1H), 8.15 (s, 1H), 7.83-7.14 (m, 9H), 7.05-6.48 (m, 2H), 6.14 (bs, 2H), 4.70 (s, 2H), 4.20 (s, 2H), 3.90 (s, 2H). MS (EI) for C₂₄H₂ᵢN₅O₂ found 412.5 (MH+).

1-Amino-N-[5-(4-{[2-ethyl-3-fluoro-4-(methylsulfonyl)-phenyl]carbonyl}-2,3,4,5-tetrahydro-1,4-benzoxazepin-7-yl)-1,3-thiazol-2-yl]cyclopropane-carboxamide. ¹H NMR (400 MHz, DMSO-d₆): δ 8.81 (s, 1H), 7.92-6.79 (m, 6H), 4.89 (m, 1H), 4.44-3.92 (m, 4H), 3.65 (m, 5H), 3.31 (s, 3H), 2.63-2.02 (m, 2H), 1.62 (m, 2H), 1.43 (m, 2H), 1.09 and 1.01 (t, 3H). MS (EI) for C₂₆H₂₇FN₄O₅S₂, found 459 (MH+).

1-Amino-N-[5-(4-{[2-ethyl-3-fluoro-4-(methylsulfonyl)phenyl]carbonyl}-2,3,4,5-tetrahydro-1,4-benzoxazepin-7-yl)-1,3-thiazol-2-yl]cyclobutanecarboxamide. ¹H NMR (400 MHz, DMSO-d₆): δ 8.89 (s, 1H), 7.96-6.81 (m, 6H), 4.88 (m, 1H), 4.51-3.96 (m, 5H), 3.64 (s, 3H), 3.41 (m, 6H), 2.79-1.99 (m, 2H), 1.17 and 0.96 (t, 3H). MS (EI) for C₂₇H₂₉FN₄O₅S₂, found 473 (MH+).

N-[5-(4-{[2-Ethyl-3-fluoro-4-(methylsulfonyl)phenyl]carbonyl}-2,3,4,5-tetrahydro-1,4-benzoxazepin-7-yl)-1,3-thiazol-2-yl]-N²-methyl-L-alaninamide. ¹H NMR (400 MHz, DMSO-d₆): δ 9.28 (s, 1H), 9.12 (s, 1H), 7.94-6.76 (m, 6H), 4.95 (m, 1H), 4.44-3.89 (m, 5H), 3.56 (m, 1H), 3.41 (m, 3H), 2.59 (m, 3H), 2.77-1.99 (m, 2H), 1.54 (m, 3H), 1.13 and 0.96 (t, 3H). MS (EI) for C₂₆H₂₉FN₄O₅S₂, found 562 (MH+).

N-[5-(4-{[2-Ethyl-3-fluoro-4-(methylsulfonyl)-phenyl]carbonyl}-2,3,4,5-tetrahydro-1,4-benzoxazepin-7-yl)-1,3-thiazol-2-yl]-N²-methyl-D-alaninamide. ¹H NMR (400 MHz, DMSO-d₆): δ 9.28 (s, 1H), 9.12 (s, 1H), 7.94-6.76 (m, 6H), 4.95 (m, 1H), 4.44-3.89 (m, 5H), 3.56 (m, 1H), 3.41 (m, 3H), 2.59 (m, 3H), 2.77-1.99 (m, 2H), 1.54 (m, 3H), 1.13 and 0.96 (t, 3H). MS (EI) for C₂₆H₂₉FN₄O₅S₂, found 562 (MH+).

6-(4-{[2-Ethyl-3-fluoro-4-(methylsulfonyl)phenyl]carbonyl}-2,3,4,5-tetrahydro-1,4-benzoxazepin-7-yl)imidazo[1,2-c]pyridin-2-amine. ¹H NMR (400 MHz, DMSO-d₆): δ 9.0 (s, 1H), 8.42-8.71 (m, 2H), 7.82-7.32 (m, 4H), 7.21-6.98 (m, 2H), 4.93 (d.d, 1H), 4.48 (s, 1H), 4.35 (m, 1H), 4.20 (m, 1H), 4.18 (m, 1H), 3.60 (m, 1H), 2.65-2.09 (m, 2H), 3.49 (s, 3H), 1.08 and 0.97 (t, 3H). MS (EI) for C₂₆H₂₅FN₄O₄S, found 510 (MH+).

4-(4-{[2-Ethyl-3-fluoro-4-(methylsulfonyl)phenyl]carbonyl}-2,3,4,5-tetrahydro-1,4-benzoxazepin-7-yl)pyridin-2-amine. ¹H NMR (400 MHz, DMSO-d₆): δ 7.98-6.43 (m, 8H), 5.96 (s, 1H), 5.86 (s, 1H), 4.87 (m, 1H), 4.45-4.0 (m, 4H), 3.59 (s, 1H), 3.37 (s, 3H), 2.64-2.02 (m, 2H), 1.18 and 0.96 (t, 3H). MS (EI) for C₂₄H₂₄FN₃O₄S, found 470 (MH+).

5-(4-{[2-Ethyl-3-fluoro-4-(methylsulfonyl)phenyl]carbonyl}-2,3,4,5-tetrahydro-1,4-benzoxazepin-7-yl)pyridin-3-amine. ¹H NMR (400 MHz, DMSO-d₆): δ 8.04-6.73 (m, 8H), 5.4 (m, 2H), 4.9 (m, 1H), 4.49-3.97 (m, 4H), 3.6 (m, 1H), 3.4 (s, 3H), 2.69-2.03 (m, 2H), 1.21 and 0.96 (t, 3H). MS (EI) for C₂₄H₂₄FN₃O₄S, found 470 (MH+).

3-Chloro-5-(4-{[2-ethyl-3-fluoro-4-(methylsulfonyl)-phenyl]carbonyl}-2,3,4,5-tetrahydro-1,4-benzoxazepin-7-yl)pyridin-2-amine. ¹H NMR (400 MHz, DMSO-d₆): δ 8.28-6.82 (m, 7H), 6.4 (s, 2H), 4.9 (m, 1H), 4.42 (m, 1H), 4.32-4.02 (m, 3H), 3.55 (m, 1H), 3.4 (s, 3H), 2.73-2.18 (m, 2H), 1.21 and 1.01 (t, 3H). MS (EI) for C₂₄H₂₃ClF N₃O₄S, found 504 (MH+).

5-(4-{[2-Ethyl-3-fluoro-4-(methylsulfonyl)phenyl]carbonyl}-2,3,4,5-tetrahydro-1,4-benzoxazepin-7-yl)-3-[(phenylmethyl)oxy]pyridin-2-amine. ¹H NMR (400 MHz, DMSO-d₆): δ 7.84-6.79 (m, 13H), 5.84 (d, 2H), 5.2 (d, 2H), 4.89 (m, 1H), 4.40 (s, 1H), 4.27-4.02 (m, 4H), 3.58 (m, 1H), 3.36 (s, 3H), 2.68-2.18 (m, 2H), 1.17 and 1.01 (t, 3H). MS (EI) for C₃₁H₃₀FN₃O₅S, found 576 (MH+).

2-Amino-5-(4-{[2-ethyl-3-fluoro-4-(methylsulfonyl)-phenyl]carbonyl}-2,3,4,5-tetrahydro-1,4-benzoxazepin-7-yl)pyridin-3-ol. ¹H NMR (400 MHz, DMSO-d₆): δ 9.6 (d, 1H), 7.74-6.68 (m, 7H), 5.61 (s, 2H), 4.97 (m, 1H), 4.43-3.97 (m, 4H), 3.56 (s, 1H), 3.31 (s, 3H), 2.68-2.12 (m, 2H), 1.18 and 1.01 (t, 3H). MS (EI) for C₂₄H₂₄FN₃O₅S, found 486 (MH+).

4-{[2-Ethyl-3-fluoro-4-(methylsulfonyl)phenyl]carbonyl}-7-[5-(1H-imidazol-2-yl)-3-thienyl]-2,3,4,5-tetrahydro-1,4-benzoxazepine. ¹H NMR (400 MHz, DMSO-d₆): δ 7.96-6.84 (m, 9H), 4.97 (m, 1H), 4.42-3.97 (m, 4H), 3.61 (s, 1H), 3.32 (s, 3H), 2.42-2.08 (m, 2H), 1.08 and 0.98 (t, 3H). MS (EI) for C₂₆H₂₄FN₃O₅S₂, found 526 (MH+).

4-{[2-Ethyl-4-(ethylsulfonyl)-3-fluorophenyl]carbonyl}-7-[5-(1H-imidazol-2-yl)-3-thienyl]-2,3,4,5-tetrahydro-1,4-benzoxazepine. ¹H NMR (400 MHz, DMSO-d₆): δ 7.96-6.77 (m, 9H), 4.97 (m, 1H), 4.42 (s, 1H), 4.40-4.01 (m, 3H), 3.61 (s, 1H), 3.42 (m, 2H), 2.66-2.15 (m, 2H), 1.17 and 0.96 (t, 6H). MS (EI) for C₂₇H₂₆FN₃O₄S₂, found 540 (MH+).

7-[3-Chloro-4-(1H-imidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 12.4 (s, 1H), 7.92-6.98 (m, 10H), 4.97 (m, 1H), 4.44 (s, 1H), 4.38-4.02 (m, 3H), 3.61 (s, 1H), 3.32 (s, 3H), 2.67-2.04 (m, 2H), 1.18 and 1.02 (t, 3H). MS (EI) for C₂₈H₂₅ClFN₃O₄S, found 554 (MH+).

7-[3-Chloro-4-(1H-imidazol-2-yl)phenyl]-4-{[2-ethyl-4-(ethylsulfonyl)-3-fluorophenyl]carbonyl}-2,3,4,5-tetrahydro-1,4-benzoxazepine. ¹H NMR (400 MHz, DMSO-d₆): δ 7.87-6.85 (m, 10H), 4.97 (m, 1H), 4.44 (s, 1H), 4.36-4.02 (m, 3H), 3.61 (s, 1H), 3.41 (m, 2H), 2.67-2.12 (m, 2H), 1.18 and 0.98 (m, 6H). MS (EI) for C₂₉H₂₇ClFN₃O₄S, found 569 (MH+).

4-{[2-Ethyl-3-fluoro-4-(methylsulfonyl)phenyl]carbonyl}-7-[3 (trifluoromethyl)phenyl]-2,3,4,5-tetrahydro-1,4-benzoxazepine. ¹H NMR (400 MHz, DMSO-d₆): δ 8.01-6.94 (m, 9H), 4.97 (m, 1H), 4.44 (s, 1H), 4.36-4.13 (m, 3H), 3.61 (m, 1H), 3.41 (s, 3H), 2.67-2.13 (m, 2H), 1.13 and 1.01 (t, 3H). MS (EI) for C₂₆H₂₃F₄NO₄S, found 522 (MH+).

4-{[2-Ethyl-3-fluoro-4-(methylsulfonyl)phenyl]carbonyl}-7-{3-[(trifluoromethyl)oxy]phenyl}-2,3,4,5-tetrahydro-1,4-benzoxazepine. ¹H NMR (400 MHz, DMSO-d₆): δ 7.81-6.88 (m, 9H), 4.95 (m, 1H), 4.42 (s, 1H), 4.38-4.05 (m, 3H), 3.62 (m, 1H), 3.38 (s, 3H), 2.67-2.14 (m, 2H), 1.12 and 1.0 (t, 3H). MS (EI) for C₂₆H₂₃F₄NO₅S, found 538 (MH+).

4-(4-{[2-Ethyl-3-fluoro-4-(methylsulfonyl)phenyl]carbonyl}-2,3,4,5-tetrahydro-1,4-benzoxazepin-7-yl)-1,3-thiazol-2-amine. ¹H NMR (400 MHz, DMSO-d₆): δ 7.81-6.68 (m, 6H), 4.87 (m, 1H), 4.42-3.91 (m, 4H), 3.61 (s, 1H), 3.38 (s, 3H), 2.65-1.99 (m, 2H), 1.12 and 0.98 (t, 3H). MS (EI) for C₂₂H₂₂FN₃O₄S₂, found 476 (MH+).

5-[4-({3-Fluoro-4-[(fluoromethyl)sulfonyl]-2-methylphenyl}-carbonyl)-2,3,4,5-tetrahydro-1,4-benzoxazepin-7-yl]pyridin-2-amine. ¹H NMR (400 MHz, DMSO-d₆): δ 8.24-6.48 (m, 6H), 6.05 (d, 2H), 5.8 (m, 2H), 4.87 (m, 1H), 4.5-4.01 (m, 4H), 3.57 (m, 1H), 1.96 (d, 3H). MS (EI) for C₂₃H₂₁F₂N₃O₄S, found 474 (MH+).

7-(1H-Benzimidazol-6-yl)-4-({3-fluoro-4-[(fluoromethyl)-sulfonyl]-2-methylphenyl}carbonyl)-2,3,4,5-tetrahydro-1,4-benzoxazepine. ¹H NMR (400 MHz, DMSO-d₆): δ 12.3 (m, 1H), 8.4-6.73 (m, 9H), 5.74-6.0 (m, 2H), 4.94 (m, 1H), 4.54-4.41 (m, 5H), 3.61 (m, 1H), 2.03 (d, 3H). MS (EI) for C₂₅H₂₁F₂N₃O₄S, found 498 (MH+).

7-(1H-Indazol-6-yl)-4-{[4-(methylsulfonyl)phenyl]carbonyl}-2,3,4,5-tetrahydro-1,4-benzoxazepine. ¹H NMR (400 MHz, DMSO-d₆): δ 13.02 (br.s, 1H), 8.12-7.96 (m, 3H), 7.83-7.52 (m, 3H), 7.41 (m, 1H), 7.37-7.17 (m, 4H), 4.91 (s, 1H), 4.59 (s, 1H), 4.37 (s, 1H), 4.19 (s, 1H), 3.35 (s, 3H). MS (EI) for C₂₄H₂₁N₃O₄S, found 448 (MH+).

7-(2-Methyl-1H-benzimidazol-6-yl)-4-{[4-(methylsulfonyl)-phenyl]carbonyl}-2,3,4,5-tetrahydro-1,4-benzoxazepine. ¹H NMR (400 MHz, DMSO-d₆): δ 8.07-6.83 (m, 10H), 4.78 (s, 1H), 4.53-4.0 (m, 4H), 3.72 (s, 1H), 3.29 (s, 3H), 3.21 (s, 3H). MS (EI) for C₂₅H₂₃N₃O₄S, found 462 (MH+).

[2-Ethyl-3-(methylamino)-4-(methylsulfonyl)phenyl]{7-[2-(methylamino)pyrimidin-5-yl]-2,3-dihydro-1,4-benzoxazepin-4(5H)-yl}methanone. ¹H NMR (400 MHz, CDCl₃): δ 8.53 (s, 1H), 8.35 (s, 1H), 7.75 (dd, 1H), 7.54 (d, 0.5H), 7.37-7.30 (m, 1H), 7.13 (dd, 1H), 6.72 (dd, 1H), 6.61 (d, 0.5H), 5.56-5.48 (m, 1H), 5.24-5.16 (m, 1H), 4.91 (dd, 1H), 4.46-3.93 (m, 4H), 3.68-3.62 (m, 1H), 3.11-3.00 (m, 6H), 2.83-2.32 (m, 2H), 1.29-1.10 (m, 3H). MS (EI) for C25H29N5O4S, found 496 (MH+).

[2-Ethyl-3-fluoro-4-(methylsulfonyl)phenyl]{7-[2-(methylthio)-pyrimidin-5-yl]-2,3-dihydro-1,4-benzoxazepin-4(5H)-yl}methanone. ¹H NMR (400 MHz, CDCl₃): δ 8.74 (s, 1H), 8.54 (s, 1H), 7.87-7.80 (m, 1H), 7.60 (d, 0.5H), 7.45-7.38 (m, 1H), 7.18 (dd, 1H), 7.07 (dd, 1H), 6.64 (d, 0.5H), 4.92 (dd, 1H), 4.50-3.98 (m, 4H), 3.67-3.62 (m, 1H), 3.26 (d, 3H), 2.82-2.31 (m, 2H), 2.61 (d, 3H), 1.17 (tt, 3H). MS (EI) for C₂₄H₂₄FN₃O₄S₂, found 502 (MH+).

[2-Ethyl-3-fluoro-4-(methylsulfonyl)phenyl]{7-[2-(methylsulfonyl)pyrimidin-5-yl]-2,3-dihydro-1,4-benzoxazepin-4(5H)-yl}methanone. ¹H NMR (400 MHz, CDCl₃): δ 9.12 (s, 1H), 8.93 (s, 1H), 7.86-7.79 (m, 1H), 7.70 (d, 0.5H), 7.56-7.48 (m, 1H), 7.29-23 (m, 1H), 7.06 (dd, 1H), 6.77 (d, 0.5H), 4.96 (dd, 1H), 4.56-4.03 (m, 4H), 3.70-3.64 (m, 1H), 3.42 (s, 3H), 3.25 (d, 3H), 2.83-2.36 (m, 2H), 1.23-1.14 (m, 3H). MS (EI) for C₂₄H₂₄FN₃O₆S₂, found 534 (MH+).

[7-(2-Ethoxypyrimidin-5-yl)-2,3-dihydro-1,4-benzoxazepin-4(5H)-yl][2-ethyl-3-fluoro-4-(methylsulfonyl)phenyl]methanone. ¹H NMR (400 MHz, CDCl₃): δ 8.71 (s, 1H), 8.51 (s, 1H), 7.87-7.80 (m, 1H), 7.58 (d, 0.5H), 7.43-7.36 (m, 1H), 7.20-7.14 (m, 1H), 7.09-7.04 (m, 1H), 6.62 (d, 0.5H), 4.92 (dd, 1H), 4.51-3.96 (m, 6H), 3.67-3.62 (m, 1H), 3.25 (dd, 3H), 2.82-2.30 (m, 2H), 1.50-1.44 (m, 3H), 1.23-1.12 (m, 3H). MS (EI) for C₂₅H₂₆FN₃O₅S, found 500 (MH+).

[7-(2-{[3-(Dimethylamino)propyl]amino}pyrimidin-5-yl)-2,3-dihydro-1,4-benzoxazepin-4(5H)-yl][2-ethyl-3-fluoro-4-(methylsulfonyl)phenyl]methanone. ¹H NMR (400 MHz, CDCl₃): δ 8.50 (s, 1H), 8.30 (s, 1H), 7.87-7.79 (m, 1H), 7.52 (d, 0.5H), 7.37-7.30 (m, 1H), 7.17-7.04 (m, 2H), 6.66-6.60 (m, 1H), 6.54 (d, 0.5H), 6.37 (bs, 1H), 4.90 (dd, 1H), 4.46-3.94 (m, 4H), 3.66-3.60 (m, 1H), 3.57-3.48 (m, 2H), 3.26 (d, 3H), 2.81-2.73 (m, 2H), 2.70-2.29 (m, 2H), 2.50 (d, 6H), 2.01-1.92 (m, 2H), 1.18 (tt, 3H). MS (EI) for C₂₈H₃₄FN₅O₄S, found 556 (MH+).

[7-(2-{[2-(Dimethylamino)ethyl]amino}pyrimidin-5-yl)-2,3-dihydro-1,4-benzoxazepin-4(5H)-yl][2-ethyl-3-fluoro-4-(methylsulfonyl)phenyl]methanone. ¹H NMR (400 MHz, CDCl₃): δ 8.58 (bs, 1H), 8.50 (s, 1H), 8.30 (s, 1H), 7.87-7.79 (m, 1H), 7.52 (d, 0.5H), 7.37-7.30 (m, 1H), 7.16-7.04 (m, 2H), 6.66-6.60 (m, 1H), 6.53 (d, 0.5H), 4.90 (dd, 1H), 4.47-3.95 (m, 4H), 3.75-3.69 (m, 2H), 3.66-3.60 (m, 1H), 3.26 (d, 3H), 2.93 (q, 2H), 2.82-2.30 (m, 2H), 2.55 (d, 6H), 1.18 (tt, 3H). MS (EI) for C₂₇H₃₂FN₅O₄S, found 542 (MH+).

1-(4-{[7-(1H-Indazol-6-yl)-2,3-dihydro-1,4-benzoxazepin-4(5H)-yl]carbonyl}-phenyl)ethanone. ¹H NMR (400 MHz, DMSO-d₆): δ 13.14-13.10 (d, 1H), 8.09-7.77 (m, 4H), 7.75-7.53 (m, 4H), 7.44-6.92 (m, 3H), 4.89-4.55 (d, 2H), 4.31-4.16 (m, 2H), 4.04-3.73 (m, 2H), 2.61 (s, 3H). MS (EI) for C₂₅H₂ᵢN₃O₃: 412.0 (MH⁺).

4-({7-[2-(Ethylamino)pyrimidin-4-yl]-2,3-dihydro-1,4-benzoxazepin-4(5H)-yl}carbonyl)benzenesulfonamide. ¹H NMR (400 MHz, DMSO-d₆): δ 8.33-8.27 (m, 1H), 8.14-7.85 (m, 4H), 7.60-7.22 (m, 5H), 7.12-6.89 (m, 2H), 4.88-4.55 (d, 2H), 4.36-4.21 (m, 2H), 4.03-3.72 (m, 2H), 1.18-1.12 (m, 3H). MS (EI) for C₂₂H₂₃N₅O₄S: 454.0 (MH⁺).

1-[4-({7-[2-(Ethylamino)pyrimidin-4-yl]-2,3-dihydro-1,4-benzoxazepin-4(5H)-yl}carbonyl)phenyl]-2,2,2-trifluoroethanone. ¹H NMR (400 MHz, DMSO-d₆): δ 8.33-8.24 (m, 1H), 8.14-7.95 (m, 3H), 7.66-7.41 (m, 3H), 7.33-6.85 (m, 4H), 4.87-4.53 (d, 2H), 4.33-4.22 (m, 2H), 4.02-3.75 (m, 2H), 1.18-1.12 (m, 3H). MS (EI) for C₂₄H₂ᵢF₃N₄O₃: 489.1 (MH⁺).

[4-(Methylsulfonyl)phenyl]{7-[2-(phenylamino)pyrimidin-4-yl]-2,3-dihydro-1,4-benzoxazepin-4(5H)-yl}methanone. ¹H NMR (400 MHz, DMSO-d₆): δ 8.55-8.46 (dd, 1H), 8.26-8.08 (m, 1H), 8.00-7.66 (m, 6H), 7.34-6.94 (m, 7H), 4.92-4.60 (d, 2H), 4.41-4.24 (m, 2H), 4.06-3.74 (m, 2H), 3.26-3.20 (d, 3H). MS (EI) for C₂₇H₂₄N₄O₄S: 501.0 (MH⁺).

6-(4-{[4-(Methylsulfonyl)phenyl]carbonyl}-2,3,4,5-tetrahydro-1,4-benzoxazepin-7-yl)-1,4-dihydroquinoxaline-2,3-dione. ¹H NMR (400 MHz, DMSO-d₆): δ 10.67 (bs, 2H), 8.04-7.97 (m, 2H), 7.67-7.58 (m, 2H), 7.51-7.39 (m, 3H), 7.22-6.75 (m, 3H), 4.86-4.50 (d, 2H), 4.30-4.14 (m, 2H), 4.05-3.71 (m, 2H), 3.30-3.26 (d, 3H). MS (EI) for C₂₅H₂ᵢN₃O₆S: 491.9 (MH⁺).

[7-(4-Amino-3-nitrophenyl)-2,3-dihydro-1,4-benzoxazepin-4(5H)-yl][4-(methylsulfonyl)phenyl]methanone. ¹H NMR (400 MHz, DMSO-d₆): δ 8.21-7.78 (m, 3H), 7.67-7.49 (m, 5H), 7.14-6.86 (m, 2H), 4.86-4.51 (d, 2H), 4.28-4.13 (m, 2H), 4.04-3.71 (m, 2H), 3.27-3.26 (d, 3H). MS (EI) for C₂₃H₂ᵢN₃O₆S: 465.9 (MH⁻).

7-(3-Amino-4-methylphenyl)-2,3-dihydro-1,4-benzoxazepin-4(5H)-yl][3-fluoro-2-methyl-4-(methylsulfonyl)phenyl]methanone. ¹H NMR (400 MHz, DMSO-d₆): δ 7.80-7.70 (m, 1H), 7.53-7.20 (m, 3H), 7.06-6.91 (m, 2H), 6.76-6.49 (m, 3H), 4.95-4.82 (m, 3H), 4.49-3.96 (m, 4H), 3.56 (m, 1H), 2.11-1.73 (m, 6H). MS (EI) for C₂₅H₂₅N₂O₄S, found 469 (MH+).

7-(3-Amino-4-methylphenyl)-2,3-dihydro-1,4-benzoxazepin-4(5H)-yl][2-ethyl-4-(methylsulfonyl)phenyl]methanone. ¹H NMR (400 MHz, DMSO-d₆): δ 7.89-7.76 (m, 2H), 7.55-7.30 (m, 2H), 7.05-6.91 (m, 2H), 6.76-6.41 (m, 2H), 4.99-4.76 (m, 3H), 4.34-4.01 (m, 4H), 3.53 (m, 1H), 3.26 (m, 3H), 2.33-2.15 (m, 1H), 2.07 (m, 3H), 1.15-0.98 (m, 3H). MS (EI) for C₂₆H₂₈N₂O₄S, found 465 (MH+).

7-(3-Amino-4-methylphenyl)-2,3-dihydro-1,4-benzoxazepin-4(5H)-yl][2-bromo-4-(methylsulfonyl)phenyl]methanone. ¹H NMR (400 MHz, DMSO-d₆): δ 8.26-8.20 (m, 1H), 8.03-7.95 (m, 1H), 7.63-7.34 (m, 3H), 7.06-6.91 (m, 3H), 6.76-6.49 (m, 2H), 4.95-4.76 (m, 3H), 4.46-4.00 (m, 4H), 3.56 (m, 1H), 3.32 (s, 3H), 2.06 (d, 3H). MS (EI) for C₂₄H₂₃BrN₂O₄S, found 516 (MH+).

[7-(2-Cyclopropyl-1H-benzimidazol-6-yl)-2,3-dihydro-1,4-benzoxazepin-4(5H)-yl][4-(methylsulfonyl)phenyl]methanone. ¹H NMR (400 MHz, DMSO-d₆): δ 12.27 (bs, 1H), 8.02-7.97 (m, 2H), 7.68-7.42 (m, 6H), 7.39-6.80 (m, 2H), 4.87-4.52 (d, 2H), 4.27-4.14 (m, 2H), 4.04-3.72 (m, 2H), 3.27 (bs, 3H), 2.16-2.09 (m, 1H), 1.08-1.04 (m, 4H). MS (EI) for C₂₇H₂₅N₃O₄S: 487.9 (MH⁺).

[7-(2-Cyclopropyl-1H-benzimidazol-6-yl)-2,3-dihydro-1,4-benzoxazepin-4(5H)-yl][2-ethyl-4-(ethylsulfonyl)-3-fluorophenyl]methanone. ¹H NMR (400 MHz, DMSO-d₆): δ 12.25 (m, 1H), 7.73-6.67 (m, 8H), 5.03-4.02 (m, 5H), 3.59-3.43 (m, 3H), 2.66 (m, 1H), 2.33-2.10 (m, 1H), 1.24-0.94 (m, 10H). MS (EI) for C₃₀H₃₀FN₃O₄S, found 548 (MH+).

[7-(2-Ethyl-1H-benzimidazol-6-yl)-2,3-dihydro-1,4-benzoxazepin-4(5H)-yl][4-(methylsulfonyl)phenyl]methanone. ¹H NMR (400 MHz, DMSO-d₆): δ 12.22 (bs, 1H), 8.02-7.97 (m, 2H), 7.68-7.51 (m, 6H), 7.42-6.82 (m, 2H), 4.88-4.53 (d, 2H), 4.28-4.15 (m, 2H), 4.05-3.72 (m, 2H), 3.27 (bs, 3H), 2.88-2.81 (m, 2H), 1.36-1.31 (m, 3H). MS (EI) for C₂₆H₂₅N₃O₄S: 475.9 (MH⁺).

[7-(2-Ethyl-1H-benzimidazol-6-yl)-2,3-dihydro-1,4-benzoxazepin-4(5H)-yl][2-ethyl-4-(ethylsulfonyl)-3-fluorophenyl]methanone. ¹H NMR (400 MHz, DMSO-d₆): δ 12.23 (m, 1H), 7.74-7.09 (m, 7H), 6.69 (m, 1H), 5.04-4.02 (m, 5H), 3.60-3.43 (m, 3H), 2.86-2.66 (m, 2H), 2.33-2.06 (m, 2H), 1.33-0.97 (m, 9H). MS (EI) for C₂₉H₃₀FN₃O₄S, found 536 (MH+).

[7-(2-Ethyl-1H-benzimidazol-6-yl)-2,3-dihydro-1,4-benzoxazepin-4(5H)-yl][2-ethyl-4-(methylsulfonyl)-3-fluorophenyl]methanone. ¹H NMR (400 MHz, DMSO-d₆): δ 12.26 (m, 1H), 7.79-6.77 (m, 7H), 5.04-3.59 (m, 6H), 2.83 (m, 2H), 2.36-2.06 (m, 2H), 1.35-0.96 (m, 6H). MS (EI) for C₂₈H₂₈FN₃O₄S, found 522 (MH+).

[7-(2-Ethyl-1H-benzimidazol-6-yl)-2,3-dihydro-1,4-benzoxazepin-4(5H)-yl][4-(trifluoromethyl)phenyl]methanone. ¹H NMR (400 MHz, DMSO-d₆): δ 12.27 (br. s, 1H), 7.82 (m, 2H), 7.67-7.40 (m, 6H), 7.16-6.82 (m, 2H), 4.87 (s, 1H), 4.53 (br. s, 1H), 4.15 (br. s, 1H), 4.04 (br. s, 1H), 3.72 (br. s, 1H), 2.84 (m, 2H), 1.33 (m, 3H). MS (EI) for C₂₆H₂₂F₃N₃O₂, found 466 (MH+).

5-(4-{[2-Ethyl-3-fluoro-4-(methylsulfonyl)phenyl]carbonyl}-2,3,4,5-tetrahydro-1,4-benzoxazepin-7-yl)-1,3-dihydro-2H-benzimidazol-2-one. ¹H NMR (400 MHz, DMSO-d₆): δ 10.71 (m, 2H), 7.78-7.42 (m, 2H), 7.29-6.66 (m, 6H), 5.02-4.77 (m, 1H), 4.46-4.03 (m, 4H), 3.58 (m, 1H), 2.66 (m, 1H), 2.34-2.04 (m, 1H), 1.13-0.96 (m, 3H). MS (EI) for C₂₆H₂₄FN₃O₅S, found 510 (MH+).

{7-[2-(Difluoromethyl)-1H-benzimidazol-6-yl]-2,3-dihydro-1,4-benzoxazepin-4(5H)-yl}[4-(methylsulfonyl)phenyl]methanone. ¹H NMR (400 MHz, DMSO-d₆): δ 12.85 (bs, 1H), 7.99-7.95 (m, 2H), 7.83-7.30 (m, 7H), 7.26-6.84 (m, 2H), 4.86-4.51 (d, 2H), 4.28-4.13 (m, 2H), 4.04-3.69 (m, 2H), 3.24 (s, 3H). MS (EI) for C₂₅H₂₁F₂N₃O₄S: 497.9 (MH⁺).

[4-(Methylsulfonyl)phenyl]{7-[2-(trifluoromethyl)-1H-benzimidazol-6-yl]-2,3-dihydro-1,4-benzoxazepin-4(5H)-yl}methanone. ¹H NMR (400 MHz, DMSO-d₆): δ 12.27 (bs, 1H), 7.98-7.48 (m, 8H), 7.39-6.86 (m, 2H), 4.87-4.52 (d, 2H), 4.28-4.14 (m, 2H), 4.03-3.71 (m, 2H), 3.25-3.24 (d, 3H). MS (EI) for C₂₅H₂₀F₃N₃O₄S: 515.8 (MH⁺).

{7-[2-(Fluoromethyl)-1H-benzimidazol-6-yl]-2,3-dihydro-1,4-benzoxazepin-4(5H)-yl}[4-(methylsulfonyl)phenyl]methanone. ¹H NMR (400 MHz, DMSO-d₆): δ 12.95-12.82 (m, 1H), 8.02-7.98 (m, 2H), 7.73-7.66 (m, 3H), 7.57-7.51 (m, 3H), 7.31-6.86 (m, 2H), 5.70-5.57 (m, 2H), 4.89-4.54 (m, 2H), 4.29-4.16 (m, 2H), 4.07-3.72 (m, 2H), 3.27 (s, 3H). MS (EI) for C₂₅H₂₂FN₃O₄S: 479.9 (MH⁺).

[7-(1H-benzotriazol-6-yl)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.09-7.61 (m, 5H), 7.42-6.89 (m, 3H), 5.07-4.83 (m, 4H), 3.60 (m, 1H), 3.35 (m, 3H), 2.68 (m, 1H), 2.33-2.06 (m, 1H), 1.12-0.97 (m, 3H). MS (EI) for C₂₅H₂₃FN₄O₄S, found 495 (MH+).

{7-[2-(Methylamino)-1H-benzimidazol-6-yl]-2,3-dihydro-1,4-benzoxazepin-4(5H)-yl}[4-(methylsulfonyl)phenyl]methanone. ¹H NMR (400 MHz, DMSO-d₆): δ 11.02-10.84 (bs, 2H), 8.03-7.97 (m, 2H), 7.67-7.36 (m, 4H), 7.19-6.59 (m, 4H), 4.85-4.50 (d, 2H), 4.26-4.11 (m, 2H), 4.04-3.70 (m, 2H), 3.30-3.26 (d, 3H), 2.88-2.87 (d, 3H). MS (EI) for C₂₅H₂₄N₄O₄S: 476.9 (MH⁺).

[7-(2-Chloro-1H-benzimidazol-6-yl)-2,3-dihydro-1,4-benzoxazepin-4(5H)-yl][4-(methylsulfonyl)phenyl]methanone. ¹H NMR (400 MHz, DMSO-d₆): δ 13.32 (bs, 1H), 8.01-7.95 (m, 2H), 7.73-7.50 (m, 6H), 7.29-6.86 (m, 2H), 4.88-4.53 (d, 2H), 4.29-4.16 (m, 2H), 4.05-3.72 (m, 2H), 3.26 (s, 3H). MS (EI) for C₂₄H₂₀ClN₃O₄S: 481.9 (MH⁺).

{7-[2-(Fluoromethyl)-1H-benzimidazol-6-yl]-2,3-dihydro-1,4-benzoxazepin-4(5H)-yl}[4-(trifluoromethyl)phenyl]methanone. ¹H NMR (400 MHz, DMSO-d₆): δ 12.90 (bs, 1H), 7.89-7.53 (m, 8H), 7.49-6.86 (m, 2H), 5.70-5.58 (d, 2H), 4.88-4.54 (d, 2H), 4.29-4.15 (m, 2H), 4.05-3.72 (m, 2H). MS (EI) for C₂₅H₁₉IF₄N₃O₂: 469.9 (MH⁺).

[7-(2-Cyclopropyl-1H-benzimidazol-6-yl)-2,3-dihydro-1,4-benzoxazepin-4(5H)-yl][4-(trifluoromethyl)phenyl]methanone. ¹H NMR (400 MHz, DMSO-d₆): δ 12.46-12.29 (m, 1H), 7.82-7.80 (m, 2H), 7.66-7.47 (m, 6H), 7.40-6.81 (m, 2H), 4.87-4.53 (d, 2H), 4.29-4.13 (m, 2H), 4.05-3.71 (m, 2H), 2.17-2.10 (m, 1H), 1.09-1.05 (m, 4H). MS (EI) for C₂₇H₂₂F₃N₃O₂: 477.9 (MH⁺).

[7-(2-Amino-1H-benzimidazol-6-yl)-2,3-dihydro-1,4-benzoxazepin-4(5H)-yl][4-(methylsulfonyl)phenyl]methanone. ¹H NMR (400 MHz, DMSO-d₆): δ 10.85 (bs, 1H), 8.02-7.97 (m, 2H), 7.67-7.43 (m, 4H), 7.35-7.16 (m, 2H), 7.11-6.74 (m, 2H), 6.28 (bs, 2H), 4.85-4.50 (d, 2H), 4.26-4.12 (m, 2H), 4.05-3.70 (m, 2H), 3.28-3.26 (d, 3H). MS (EI) for C₂₄H₂₂N₄O₄S: 462.9 (MH⁺).

[7-(2-Amino-1H-benzimidazol-6-yl)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 10.81 (br. m, 1H), 7.80-6.68 (m, 8H), 6.23 (s, 2H), 5.01-4.01 (m, 6H), 3.59 (m, 1H), 3.35 (m, 3H), 2.66 (m, 1H), 2.33-2.04 (m, 1H), 1.11-0.96 (m, 3H). MS (EI) for C₂₆H₂₅FN₄O₄S, found 509 (MH+).

[7-(2-Amino-1H-benzimidazol-6-yl)-2,3-dihydro-1,4-benzoxazepin-4(5H)-yl][2-ethyl-3-fluoro-4-(ethylsulfonyl)phenyl]methanone. ¹H NMR (400 MHz, DMSO-d₆): δ 10.75-10.59 (m, 1H), 7.72-6.58 (m, 8H), 6.20 (s, 2H), 4.99-4.09 (m, 6H), 3.57-3.39 (m, 3H), 2.65 (m, 1H), 2.32-2.02 (m, 1H), 1.15-0.94 (m, 6H). MS (EI) for C₂₇H₂₇FN₄O₄S, found 523 (MH+).

(4-Chlorophenyl){7-[2-(fluoromethyl)-1H-benzimidazol-6-yl]-2,3-dihydro-1,4-benzoxazepin-4(5H)-yl}methanone. ¹H NMR (400 MHz, DMSO-d₆): δ 12.93 (bs, 1H), 7.80-7.32 (m, 9H), 7.07-6.99 (m, 1H), 5.69-5.58 (d, 2H), 4.85-4.56 (d, 2H), 4.28-4.15 (m, 2H), 4.01-3.76 (m, 2H). MS (EI) for C₂₄H₁₉ClFN₃O₂ 436.0 (MH⁺).

{7-[2-(Fluoromethyl)-1H-benzimidazol-6-yl]-2,3-dihydro-1,4-benzoxazepin-4(5H)-yl}(4-iodophenyl)methanone. ¹H NMR (400 MHz, DMSO-d₆): δ 12.91 (bs, 1H), 7.81-7.32 (m, 7H), 7.21-6.95 (m, 3H), 5.70-5.58 (d, 2H), 4.84-4.56 (d, 2H), 4.27-4.14 (m, 2H), 4.00-3.75 (m, 2H). MS (EI) for C₂₄H₁₉FIN₃O₂ 526.0 (MH⁺).

{7-[2-(Fluoromethyl)-1H-benzimidazol-6-yl]-2,3-dihydro-1,4-benzoxazepin-4(5H)-yl}[2-methyl-4-(methylsulfonyl)phenyl]methanone. ¹H NMR (400 MHz, DMSO-d₆): δ 12.88 (bs, 1H), 7.86-7.71 (m, 3H), 7.68-7.20 (m, 5H), 7.11-6.61 (m, 1H), 5.70-5.57 (dd, 2H), 5.00-4.42 (m, 2H), 4.30-3.54 (m, 4H), 3.24-3.24 (d, 3H), 2.25-1.97 (d, 3H). MS (EI) for C₂₆H₂₄FN₃O₄S: 494.0 (MH⁺).

7-[2-(Fluoromethyl)-1H-benzimidazol-6-yl]-4-[(4-methyl-4H-furo[3,2-b]pyrrol-5-yl)carbonyl]-2,3,4,5-tetrahydro-1,4-benzoxazepine. ¹H NMR (400 MHz, DMSO-d₆): δ 7.76 (s, 1H), 7.69 (d, 1H), 7.63 (d, 1H), 7.55-7.43 (m, 3H), 7.06 (d, 1H), 6.76 (m, 1H), 6.33 (br. s, 1H), 5.67 (s, 1H), 5.56 (s, 1H), 4.86 (s, 2H), 4.28 (br. m, 2H), 4.05 (br. m, 2H), 3.62 (s, 3H). MS (EI) for C₂₅H₂ᵢF₂N₄O₃, found 445 (MH+).

4-{[2-Ethyl-4-(ethylsulfonyl)-3-fluorophenyl]carbonyl}-7-[2-(fluoromethyl)-1H-benzimidazol-6-yl]-2,3,4,5-tetrahydro-1,4-benzoxazepine. ¹H NMR (400 MHz, DMSO-d₆): δ 7.81-7.51 (m, 5H), 7.31-7.07 (m, 2H), 6.72 (s, 1H), 5.69-5.56 (m, 2H), 5.05-4.81 (m, 1H), 4.47-4.04 (m, 4H), 3.60-3.36 (m, 5H), 1.18-0.96 (m, 6H). MS (EI) for C₂₈H₂₇F₂N₃O₄S, found 540 (MH+).

4-{[2-ethyl-3-fluoro-4-(methylsulfonyl)phenyl]carbonyl}-7-[2-(fluoromethyl)-1H-benzimidazol-6-yl]-2,3,4,5-tetrahydro-1,4-benzoxazepine. ¹H NMR (400 MHz, DMSO-d₆): δ 12.87 (m, 1H), 7.80-7.53 (m, 5H), 7.30-7.08 (m, 3H), 6.80 (m, 1H), 5.70-5.57 (m, 2H), 5.05-4.81 (m, 1H), 4.51-4.02 (m, 4H), 3.60 (m, 1H), 2.68 (m, 1H), 2.34-2.07 (m, 1H), 1.23-0.97 (m, 6H). MS (EI) for C₂₇H₂₅F₂N₃O₄S, found 526 (MH+).

[2,3-Dimethyl-4-(methylsulfonyl)phenyl][7-(2-methyl-1H-benzimidazol-6-yl)-2,3-dihydro-1,4-benzoxazepin-4(5H)-yl]methanone. ¹H NMR (400 MHz, DMSO-d₆): δ 12.27-12.10 (m, 1H), 7.88-7.47 (m, 5H), 7.26-6.54 (m, 3H), 4.98-4.35 (m, 2H), 4.32-4.10 (m, 2H), 3.92-3.53 (m, 2H), 3.24-3.19 (d, 3H), 2.58-2.54 (m, 3H), 2.46-2.33 (m, 3H), 2.12-1.70 (d, 3H). MS (EI) for C₂₇H₂₇N₃O₄S₂: 489.9 (MH⁺).

[3-Fluoro-2-methyl-4-(methylsulfonyl)phenyl][7-(2-methyl-1H-benzimidazol-6-yl)-2,3-dihydro-1,4-benzoxazepin-4(5H)-yl]methanone. ¹H NMR (400 MHz, DMSO-d₆): δ 12.24 (bs, 1H), 7.70-7.67 (m, 2H), 7.55-7.40 (m, 3H), 7.30-7.22 (dd, 1H), 7.13-6.73 (m, 2H), 4.98-4.35 (m, 2H), 4.34-4.12 (m, 2H), 3.97-3.56 (m, 2H), 3.35-3.34 (d, 3H), 2.50-2.49 (d, 3H), 2.13-1.77 (dd, 3H). MS (EI) for C₂₆H₂₄FN₃O₄S: 493.9 (MH⁺).

[2,6-Dimethyl-4-(methylsulfonyl)phenyl][7-(2-methyl-1H-benzimidazol-6-yl)-2,3-dihydro-1,4-benzoxazepin-4(5H)-yl]methanone. ¹H NMR (400 MHz, CDCl₃): δ 7.77-7.20 (m, 8H), 7.15-6.31 (m, 1H), 4.97-4.28 (d, 2H), 4.25-3.55 (m, 4H), 3.17-3.03 (d, 3H), 2.76-2.71 (d, 3H), 2.25-2.07 (d, 6H). MS (EI) for C₂₇H₂₇N₃O₄S: 490.0 (MH⁺).

[7-(2-Methyl-1H-Benzimidazol-6-yl)-2,3-dihydro-1,4-benzoxazepin-4(5H)-yl][4-(methylsulfonyl)-3-(trifluoromethyl)phenyl]methanone. ¹H NMR (400 MHz, DMSO-d₆): δ 12.00 (bs, 1H), 8.31-7.99 (m, 2H), 7.91-7.44 (m, 5H), 7.27-7.03 (m, 2H), 4.89-4.55 (d, 2H), 4.35-4.15 (m, 2H), 4.06-3.73 (m, 2H), 3.34-3.31 (d, 3H), 2.54-2.53 (d, 3H). MS (EI) for C₂₆H₂₂F₃N₃O₄S: 529.9 (MH⁺).

[2,5-Difluoro-4-(methylsulfonyl)phenyl][7-(2-methyl-1H-benzimidazol-6-yl)-2,3-dihydro-1,4-benzoxazepin-4(5H)-yl]methanone. ¹H NMR (400 MHz, DMSO-d₆): δ 12.28-12.10 (m, 1H), 7.80-7.49 (m, 5H), 7.42-7.02 (m, 3H), 4.88-4.53 (m, 2H), 4.25-4.11 (m, 2H), 4.07-3.71 (m, 2H), 3.40-3.39 (m, 3H), 2.50 (s, 3H). MS (EI) for C₂₅H₂₁F₂N₃O₄S: 497.9 (MH⁺).

[3,5-Difluoro-4-(methylsulfonyl)phenyl][7-(2-methyl-1H-benzimidazol-6-yl)-2,3-dihydro-1,4-benzoxazepin-4(5H)-yl]methanone. ¹H NMR (400 MHz, DMSO-d₆): δ 12.22 (bs, 1H), 7.67-7.36 (m, 5H), 7.28-7.05 (m, 3H), 4.84-4.54 (m, 2H), 4.30-4.13 (m, 2H), 4.03-3.71 (m, 2H), 3.46-3.45 (m, 3H), 2.50 (s, 3H). MS (EI) for C₂₅H₂₁F₂N₃O₄S: 497.9 (MH⁺).

4-{[2-Fluoro-4-(methylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 7.93-7.90 (m, 1H), 7.84-7.81 (m, 1H), 7.71 (m, 2H), 7.56-7.44 (m, 3H), 7.18-7.06 (m, 1H), 6.82 (m, 1H), 4.90 (s, 1H), 4.51 (s, 1H), 4.25 (m, 1H), 4.12 (m, 2H), 3.68 (m, 1H). MS (EI) for C₂₅H₂₂FN₃O₄S, found 480 (MH+).

7-(2-Methyl-1H-benzimidazol-6-yl)-4-{[3-methyl-4-(methylsulfonyl)-phenyl]carbonyl}-2,3,4,5-tetrahydro-1,4-benzoxazepine. ¹H NMR (400 MHz, DMSO-d₆): δ 7.94 (m, 1H), 7.71 (d, 1H), 7.61-6.92 (m, 8H), 4.87 (s, 1H), 4.52 (s, 1H), 4.29 (m, 1H), 4.15 (m, 1H), 3.73 (m, 2H), 3.24 (m, 3H), 2.66 (s, 3H), 2.55 (m, 3H). MS (EI) for C₂₆H₂₅N₃O₄S, found 476 (MH+).

(1,1-Dioxido-3,4-dihydro-2H-1-benzothiopyran-6-yl)[7-(2-methyl-1H-benzimidazol-6-yl)-2,3-dihydro-1,4-benzoxazepin-4(5H)-yl]methanone. ¹H NMR (400 MHz, DMSO-d₆): δ 12.26 (bs, 1H), 7.87-7.81 (m, 1H), 7.67 (s, 1H), 7.59-7.38 (m, 5H), 7.24-6.98 (m, 2H), 4.85-4.52 (d, 2H), 4.29-4.13 (m, 2H), 4.02-3.72 (m, 2H), 3.54-3.47 (m, 2H), 3.03-2.76 (m, 2H), 2.50 (bs, 3H), 2.34-2.11 (m, 2H). MS (EI) for C₂₇H₂₅N₃O₄S: 487.9 (MH⁺).

4-[(1,1-Dioxido-2,3-dihydro-1-benzothien-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.25 (m, 1H), 7.81-7.33 (m, 8H), 7.21-6.92 (m, 2H), 4.87 (s, 1H), 4.54 (s, 1H), 4.29 (s, 1H), 4.14 (s, 1H), 4.04 (s, 1H), 3.62 (m, 1H), 3.25 (m, 1H). MS (EI) for C₂₆H₂₃N₃O₄S, found 474 (MH+).

[2-Ethyl-3-fluoro-4-(methylsulfonyl)phenyl][7-(2-methyl-1H-benzimidazol-6-yl)-2,3-dihydro-1,4-benzoxazepin-4(5H)-yl]methanone. ¹H NMR (400 MHz, DMSO-d₆): δ 12.31-12.10 (m, 1H), 7.79-7.40 (m, 5H), 7.30-6.75 (m, 3H), 5.04-4.38 (m, 2H), 4.33-3.55 (m, 4H), 3.37 (bs, 3H), 2.69-2.40 (m, 1H), 2.50 (s, 3H), 2.38-2.04 (m, 1H), 1.12-0.97 (m, 3H). MS (EI) for C₂₇H₂₆FN₃O₄S: 507.9 (MH⁺).

[2-Ethyl-4-(ethylsulfonyl)-3-fluorophenyl][7-(2-methyl-1H-benzimidazol-6-yl)-2,3-dihydro-1,4-benzoxazepin-4(5H)-yl]methanone. ¹H NMR (400 MHz, DMSO-d₆): δ 12.27-12.17 (m, 1H), 7.76-7.39 (m, 5H), 7.31-6.68 (m, 3H), 5.04-4.38 (m, 2H), 4.34-3.55 (m, 4H), 3.47-3.55 (m, 2H), 2.69-2.42 (m, 1H), 2.51-2.49 (m, 3H), 2.39-2.04 (m, 1H), 1.18-0.96 (m, 6H). MS (EI) for C₂₈H₂₈FN₃O₄S: 522.2 (MH⁺).

[5-Fluoro-2-methyl-4-(methylsulfonyl)phenyl][7-(2-methyl-1H-benzimidazol-6-yl)-2,3-dihydro-1,4-benzoxazepin-4(5H)-yl]methanone. ¹H NMR (400 MHz, DMSO-d₆): δ 12.24 (bs, 1H), 7.96-7.40 (m, 6H), 7.30-6.72 (m, 2H), 4.98-4.36 (m, 2H), 4.27-4.13 (m, 2H), 4.10-3.55 (m, 2H), 3.34 (bs, 3H), 2.49 (bs, 3H), 2.20-1.95 (d, 3H). MS (EI) for C₂₆H₂₄FN₃O₄S: 493.9 (MH⁺).

[7-(2-Methyl-1H-benzimidazol-6-yl)-2,3-dihydro-1,4-benzoxazepin-4(5H)-yl][4-methyl-6-(methylsulfonyl)pyridin-3-yl]methanone. ¹H NMR (400 MHz, DMSO-d₆): δ 12.58 (bs, 1H), 8.59-8.34 (d, 1H), 8.02-8.00 (d, 1H), 7.69-7.42 (m, 4H), 7.18-6.77 (m, 2H), 4.93-4.39 (d, 2H), 4.26-4.11 (m, 2H), 4.02-3.59 (m, 2H), 3.27-3.26 (d, 3H), 2.51-2.49 (d, 3H), 2.29-2.14 (d, 3H). MS (EI) for C₂₅H₂₄N₄O₄S: 476.9 (MH⁺).

[3-Fluoro-2-methyl-4-(methylsulfonyl)phenyl][7-{2-[(methylamino)methyl]-1H-benzimidazol-6-yl}-2,3-dihydro-1,4-benzoxazepin-4(5H)-yl]methanone. ¹H NMR (400 MHz, DMSO-d₆): δ 12.27 (bs, 1H), 7.79-7.67 (m, 2H), 7.55-7.42 (m, 3H), 7.31-6.73 (m, 3H), 4.98-4.35 (m, 2H), 4.35-3.58 (m, 4H), 3.89-3.87 (d, 2H), 3.35-3.34 (d, 3H), 2.34-2.33 (d, 3H), 2.14-1.78 (dd, 3H). MS (EI) for C₂₇H₂₇FN₄O₄S: 523.1 (MH⁺).

N,N-Dimethyl-1-[6-(4-{[4-(methylsulfonyl)phenyl]carbonyl}-2,3,4,5-tetrahydro-1,4-benzoxazepin-7-yl)-1H-benzimidazol-2-yl]methanamine. ¹H NMR (400 MHz, DMSO-d₆): δ 7.84 (m, 2H), 7.40 (m, 2H), 6.99 (m, 3H), 4.90 (m, 3H), 4.34-4.03 (m, 4H), 4.53 (br.m, 1H), 3.26 (d, 3H), 2.27 (m, 1H), 2.07 (d, 3H), 1.14-0.99 (m, 3H). MS (EI) for C₂₇H₂₈N₄O₄S.HCl, found 505 (MH+).

7-{2-[(Methyloxy)methyl]-1H-benzimidazol-6-yl}-4-{[4-(methylsulfonyl)phenyl]carbonyl}-2,3,4,5-tetrahydro-1,4-benzoxazepine. ¹H NMR (400 MHz, DMSO-d₆): δ 12.62 (m, 1H), 7.99 (m, 2H), 7.69 (m, 3H), 7.52 (m, 4H), 7.08 (m, 1H), 4.88 (S, 1H), 4.65 (m, 1H), 4.53 (S, 1H), 4.28 (m, 1H), 4.05 (m, 1H), 3.72 (m, 1H), 3.39 (m, 3H), 3.27 (m, 3H). MS (EI) for C₂₆H₂₅N₃O₅S, found 492 (MH+).

N-Methyl-1-[6-(4-{[4-(methylsulfonyl)phenyl]carbonyl}-2,3,4,5-tetrahydro-1,4-benzoxazepin-7-yl)-1H-benzimidazol-2-yl]methanamine. ¹H NMR (400 MHz, DMSO-d₆): δ 10.13 (br. s, 2H), 7.99 (m, 3H), 7.87-7.49 (m, 6H), 7.11 (m, 1H), 4.91 (s, 1H), 4.60 (m, 3H), 4.32 (br. s, 1H), 4.18 (br. s, 1H), 4.05 (br. s, 1H), 3.73 (m, 1H), 3.27 (s, 3H), 2.76 (s, 3H). MS (EI) for C₂₆H₂₆N₄O₄S.HCl, found 491 (MH+).

N-Methyl-1-[6-(4-{[4-(trifluoromethyl)phenyl]carbonyl}-2,3,4,5-tetrahydro-1,4-benzoxazepin-7-yl)-1H-benzimidazol-2-yl]methanamine. ¹H NMR (400 MHz, DMSO-d₆): δ 9.88 (br. s, 2H), 7.94-7.75 (m, 4H), 7.69-7.40 (m, 4H), 7.11-6.93 (m, 1H), 4.90 (s, 1H), 4.56 (m, 3H), 4.31 (br.m, 1H), 4.17 (br. m, 1H), 4.05 (br. m, 1H), 3.74-3.63 (m, 3H), 3.52-3.45 (m, 1H), 2.74 (s, 3H). MS (EI) for C₂₆H₂₃F₃N₄O₂.HCl, found 481 (MH+).

1-(6-{4-[(4-Chlorophenyl)carbonyl]-2,3,4,5-tetrahydro-1,4-benzoxazepin-7-yl}-1H-benzimidazol-2-yl)-N-methylmethanamine. ¹H NMR (400 MHz, DMSO-d₆): δ 9.85 (m, 1H), 9.94-7.05 (m, 8H), 4.87-3.49 (m, 8H), 2.74 (s, 3H). MS (EI) for C₂₅H₂₃ClN₄O₅, found 447 (MH+).

1-(6-{4-[(4-Bromo-2-methylphenyl)carbonyl]-2,3,4,5-tetrahydro-1,4-benzoxazepin-7-yl}-1H-benzimidazol-2-yl)-N-methylmethanamine. ¹H NMR (400 MHz, DMSO-d₆): δ 7.71-7.42 (m, 6H), 7.17-7.02 (m, 1H), 6.66 (m, 1H), 4.87 (br.m, 1H), 4.41-4.09 (m, 3H), 3.89 (m, 2H), 3.56 (br.m, 1H), 2.35 (d, 3H), 2.13-1.89 (m, 3H). MS (EI) for C₂₆H₂₅BrN₄O₂, found 505 (MH+).

1-(6-{4-[(4-Bromophenyl)carbonyl]-2,3,4,5-tetrahydro-1,4-benzoxazepin-7-yl}-1H-benzimidazol-2-yl)-N-methylmethanamine. ¹H NMR (400 MHz, DMSO-d₆): δ 12.29 (br.m, 1H), 7.65-6.94 (m, 8H), 4.83-3.34 (m, 11H), 2.34 (s, 3H). MS (EI) for C₂₅H₂₃BrN₄O₂, found 492 (MH+).

1-[6-(4-{[2-Ethyl-3-fluoro-4-(methylsulfonyl)phenyl]carbonyl}-2,3,4,5-tetrahydro-1,4-benzoxazepin-7-yl)-1H-benzimidazol-2-yl]-N-methylmethanamine. ¹H NMR (400 MHz, DMSO-d₆): δ 12.29 (br. m, 1H), 7.79-6.75 (m, 8H), 5.04-4.00 (m, 6H), 3.87 (m, 2H), 3.59 (m, 1H), 3.34 (s, 3H), 2.33-2.06 (m, 4H), 1.13-0.97 (m, 3H). MS (EI) for C₂₈H₂₉FN₄O₄S, found 537 (MH+).

1-[6-(4-{[2-Ethyl-4-(ethylsulfonyl)-3-fluorophenyl]carbonyl}-2,3,4,5-tetrahydro-1,4-benzoxazepin-7-yl)-1H-benzimidazol-2-yl]-N-methylmethanamine. ¹H NMR (400 MHz, DMSO-d₆): δ 12.29 (br. m, 1H), 7.74-6.68 (m, 8H), 5.03-4.15 (m, 5H), 3.90 (m, 2H), 3.60-3.42 (m, 3H), 2.34-2.04 (m, 4H), 1.17-0.95 (m, 6H). MS (EI) for C₂₉H₃ᵢFN₄O₄S, found 551 (MH+).

Methyl [6-(4-{[2-ethyl-4-(ethylsulfonyl)-3-fluorophenyl]-carbonyl}-2,3,4,5-tetrahydro-1,4-benzoxazepin-7-yl)-1H-benzimidazol-2-yl]carbamate. ¹H NMR (400 MHz, DMSO-d₆): δ 11.65 (bs, 2H), 7.76-7.63 (m, 2H), 7.52-7.36 (m, 3H), 7.31-6.63 (m, 3H), 5.03-4.37 (m, 2H), 4.33-3.57 (m, 4H), 3.77 (s, 3H), 3.45-3.36 (m, 2H), 2.69-2.04 (m, 2H), 1.18-0.96 (m, 6H). MS (EI) for C₂₉H₂₉FN₄O₆S: 581.2 (MH⁺).

Methyl [6-(4-{[3-Fluoro-2-methyl-4-(methylsulfonyl)-phenyl]carbonyl}-2,3,4,5-tetrahydro-1,4-benzoxazepin-7-yl)-1H-benzimidazol-2-yl]carbamate. ¹H NMR (400 MHz, DMSO-d₆): δ 11.79 (bs, 2H), 7.76 (dt, 1H), 7.64-7.63 (m, 1H), 7.51-7.36 (m, 3H), 7.30-6.69 (m, 3H), 4.97-4.35 (m, 2H), 4.35-3.57 (m, 4H), 3.76 (s, 3H), 3.35-3.34 (d, 3H), 2.13-1.77 (dd, 3H). MS (EI) for C₂₇H₂₅FN₄O₆S: 553.2 (MH⁺).

[7-{2-[(Ethylamino)methyl]-1H-benzimidazol-6-yl}-2,3-dihydro-1,4-benzoxazepin-4(5H)-yl][2-ethyl-4-(ethylsulfonyl)-3-fluorophenyl]methanone. ¹H NMR (400 MHz, DMSO-d₆): δ 12.22 (bs, 1H), 7.75-7.67 (m, 2H), 7.55-7.42 (m, 3H), 7.31-6.67 (m, 3H), 5.04-4.38 (m, 2H), 4.33-3.56 (m, 4H), 3.93-3.91 (d, 2H), 3.42 (q, 2H), 2.69-2.06 (m, 2H), 2.63-2.56 (m, 2H), 1.18-0.96 (m, 9H). MS (EI) for C₃₀H₃₃FN₄O₄S: 565.2 (MH⁺).

[7-{2-[(Ethylamino)methyl]-1H-benzimidazol-6-yl}-2,3-dihydro-1,4-benzoxazepin-4(5H)-yl][3-fluoro-2-methyl-4-(methylsulfonyl)phenyl]methanone. ¹H NMR (400 MHz, DMSO-d₆): δ 12.25 (bs, 1H), 7.75 (dt, 1H), 7.68 (d, 1H), 7.55-7.42 (m, 3H), 7.30-6.73 (m, 3H), 4.98-4.36 (m, 2H), 4.36-3.57 (m, 4H), 3.93-3.92 (d, 2H), 3.33 (bs, 3H), 2.63-2.57 (m, 2H), 2.14-1.78 (dd, 3H), 1.08-1.04 (m, 3H). MS (EI) for C₂₈H₂₉FN₄O₄S: 537.2 (MH⁺).

[2-Ethyl-4-(ethylsulfonyl)-3-fluorophenyl][7-{2-[(1S)-1-(methylamino)ethyl]-1H-benzimidazol-6-yl}-2,3-dihydro-1,4-benzoxazepin-4(5H)-yl]methanone. ¹H NMR (400 MHz, DMSO-d₆): δ 12.26 (bs, 1H), 7.75-7.67 (m, 2H), 7.55-7.42 (m, 3H), 7.31-6.67 (m, 3H), 5.04-4.38 (m, 2H), 4.33-3.58 (m, 4H), 3.96-3.89 (m, 1H), 3.42 (q, 2H), 2.69-2.07 (m, 2H), 2.26-2.25 (d, 3H), 1.42 (dd, 3H), 1.18-0.96 (m, 6H). MS (EI) for C₃₀H₃₃FN₄O₄S: 565.3 (MH⁺).

[3-Fluoro-2-methyl-4-(methylsulfonyl)phenyl][7-{2-[(1S)-1-(methylamino)ethyl]-1H-benzimidazol-6-yl}-2,3-dihydro-1,4-benzoxazepin-4(5H)-yl]methanone. ¹H NMR (400 MHz, DMSO-d₆): δ 12.21 (bs, 1H), 7.75 (dt, 1H), 7.67 (d, 1H), 7.55-7.42 (m, 3H), 7.30-6.73 (m, 3H), 4.98-4.35 (m, 2H), 4.35-3.57 (m, 4H), 3.95-3.88 (m, 1H), 3.33 (bs, 3H), 2.26-2.25 (d, 3H), 2.14-1.78 (dd, 3H), 1.42 (dd, 3H). MS (EI) for C₂₈H₂₉FN₄O₄S: 537.2 (MH⁺).

[2-Ethyl-3-fluoro-4-(methylsulfonyl)phenyl][7-(2-methoxy-1H-benzimidazol-6-yl)-2,3-dihydro-1,4-benzoxazepin-4(5H)-yl]methanone. ¹H NMR (400 MHz, DMSO-d₆): δ 11.91 (dd, 1H), 7.81-7.72 (m, 1H), 7.65-7.34 (m, 3H), 7.31-7.16 (m, 2H), 7.10-6.73 (m, 2H), 5.04-4.40 (m, 2H), 4.36-3.57 (m, 4H), 4.07-4.06 (d, 3H), 3.44-3.36 (d, 3H), 2.70-1.99 (m, 2H), 1.12-0.95 (m, 3H). MS (EI) for C₂₇H₂₆FN₃O₅S: 524.2 (MH⁺).

[7-(2-Ethoxy-1H-benzimidazol-6-yl)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 11.86 (dd, 1H), 7.81-7.72 (m, 1H), 7.66-7.32 (m, 3H), 7.33-7.16 (m, 2H), 7.10-6.73 (m, 2H), 5.04-4.40 (m, 2H), 4.53-4.46 (m, 2H), 4.36-3.57 (m, 4H), 4.07-4.06 (d, 3H), 3.44-3.36 (d, 3H), 2.70-1.99 (m, 2H), 1.42-1.37 (m, 3H), 1.12-0.96 (m, 3H). MS (EI) for C₂₈H₂₈FN₃O₅S: 538.2 (MH⁺).

[2-Ethyl-3-fluoro-4-(methylsulfonyl)phenyl]{7-[2-(hydroxymethyl)-1H-benzimidazol-6-yl]-2,3-dihydro-1,4-benzoxazepin-4(5H)-yl}methanone. ¹H NMR (400 MHz, DMSO-d₆): δ 12.36 (bs, 1H), 7.80-7.67 (m, 2H), 7.55-7.44 (m, 3H), 7.30-6.75 (m, 3H), 5.05-4.38 (m, 2H), 4.70 (d, 2H), 4.34-3.55 (m, 4H), 3.35 (s, 3H), 2.70-2.04 (m, 2H), 1.12-0.97 (m, 3H). MS (EI) for C₂₇H₂₆FN₃O₅S: 524.2 (MH⁺).

[2-Ethyl-3-fluoro-4-(methylsulfonyl)phenyl]{7-[6-(methylamino)pyridin-3-yl]-2,3-dihydro-1,4-benzoxazepin-4(5H)-yl}methanone. ¹H NMR (400 MHz, DMSO-d₆): δ 8.31-8.11 (d, 1H), 7.79-7.72 (m, 1H), 7.72-7.69 (dd, 0.5H), 7.57-7.57 (d, 0.5H), 7.45-7.40 (m, 1.5H), 7.28-7.15 (dd, 1H), 7.04-7.01 (dd, 1H), 6.65-6.64 (d, 0.5H), 6.63-6.60 (dd, 1H), 6.54-6.44 (m, 1H), 5.00-4.34 (m, 2H), 4.30-3.55 (m, 4H), 3.39-3.35 (d, 3H), 2.81-2.77 (dd, 3H), 2.68-2.01 (m, 2H), 1.12-0.96 (m, 3H). MS (EI) for C₂₅H₂₆FN₃O₄S: 484.2 (MH⁺).

{7-[6-(Ethylamino)pyridin-3-yl]-2,3-dihydro-1,4-benzoxazepin-4(5H)-yl}[2-ethyl-3-fluoro-4-(methylsulfonyl)phenyl]methanone. ¹H NMR (400 MHz, DMSO-d₆): δ 8.30-8.09 (d, 1H), 7.79-7.71 (m, 1H), 7.70-7.68 (dd, 0.5H), 7.57-7.56 (d, 0.5H), 7.44-7.39 (m, 1.5H), 7.28-7.15 (dd, 1H), 7.04-7.01 (dd, 1H), 6.65-6.64 (d, 0.5H), 6.63-6.60 (t, 1H), 6.54-6.44 (m, 1H), 5.00-4.33 (m, 2H), 4.29-3.55 (m, 4H), 3.39-3.35 (d, 3H), 3.30-3.22 (m, 2H), 2.69-2.01 (m, 2H), 1.14 (t, 3H), 1.11-0.96 (m, 3H). MS (EI) for C₂₆H₂₈FN₃O₄S: 498.2 (MH⁺).

[2-Ethyl-3-fluoro-4-(methylsulfonyl)phenyl]{7-[6-(propan-2-ylamino)pyridin-3-yl]-2,3-dihydro-1,4-benzoxazepin-4(5H)-yl}methanone. ¹H NMR (400 MHz, DMSO-d₆): δ 8.29-8.08 (d, 1H), 7.79-7.71 (m, 1H), 7.69-7.66 (dd, 0.5H), 7.56-7.56 (d, 0.5H), 7.44-7.39 (m, 1.5H), 7.28-7.15 (dd, 1H), 7.04-7.01 (dd, 1H), 6.64-6.64 (d, 0.5H), 6.53-6.44 (m, 2H), 5.00-4.33 (m, 2H), 4.29-3.55 (m, 5H), 3.39-3.35 (d, 3H), 2.68-1.99 (m, 2H), 1.17-1.13 (dd, 6H), 1.11-0.95 (m, 3H). MS (EI) for C₂₇H₃₀FN₃O₄S: 512.3 (MH⁺).

[7-(2-Aminopyrimidin-5-yl)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.55 (s, 1H), 8.34 (s, 1H), 7.72 (q, 1H), 7.48-7.44 (m, 1H), 7.26-7.09 (dd, 1H), 7.04-7.01 (dd, 1H), 7.63-6.78 (dd, 1H), 6.76-6.75 (d, 2H), 4.99-4.35 (m, 2H), 4.26-3.50 (m, 5H), 3.35-3.33 (d, 3H), 2.66-2.04 (m, 2H), 1.17-1.13 (dd, 6H), 1.09-0.96 (m, 3H). MS (EI) for C₂₃H₂₄FN₄O₄S: 471.2 (MH⁺).

[2-Ethyl-3-fluoro-4-(methylsulfonyl)phenyl][7-{6-[(2-methoxyethyl)amino]pyridin-3-yl}-2,3-dihydro-1,4-benzoxazepin-4(5H)-yl]methanone. ¹H NMR (400 MHz, DMSO-d₆): δ 8.29-8.08 (d, 1H), 7.78-7.71 (m, 1H), 7.70-7.67 (dd, 0.5H), 7.57-7.56 (d, 0.5H), 7.44-7.39 (m, 1.5H), 7.28-7.15 (dd, 1H), 7.04-7.01 (dd, 1H), 6.72-6.70 (m, 1H), 6.65-6.64 (d, 0.5H), 6.61-6.51 (dd, 1H), 5.00-4.33 (m, 2H), 4.29-3.55 (m, 4H), 3.48-3.42 (m, 4H), 3.38-3.35 (d, 3H), 3.28-3.27 (d, 3H), 2.68-2.00 (m, 2H), 1.11-0.95 (m, 3H). MS (EI) for C₂₇H₃₀FN₃O₅S: 528.2 (MH⁺).

[2-Ethyl-3-fluoro-4-(methylsulfonyl)phenyl]{7-[6-(propylamino)pyridin-3-yl]-2,3-dihydro-1,4-benzoxazepin-4(5H)-yl}methanone. ¹H NMR (400 MHz, DMSO-d₆): δ 8.29-8.08 (d, 1H), 7.79-7.71 (m, 1H), 7.69-7.67 (dd, 0.5H), 7.56-7.56 (d, 0.5H), 7.44-7.39 (m, 1.5H), 7.28-7.15 (dd, 1H), 7.04-7.01 (dd, 1H), 6.67-6.65 (m, 1H), 6.64-6.64 (d, 0.5H), 6.55-6.46 (dd, 1H), 5.00-4.33 (m, 2H), 4.29-3.55 (m, 4H), 3.48-3.42 (m, 4H), 3.38-3.35 (d, 3H), 3.28-3.27 (d, 3H), 2.68-2.00 (m, 2H), 1.11-0.95 (m, 3H). MS (EI) for C₂₇H₃₀FN₃O₅S: 528.2 (MH⁺).

{7-[6-(Cyclopentylamino)pyridin-3-yl]-2,3-dihydro-1,4-benzoxazepin-4(5H)-yl}[2-ethyl-3-fluoro-4-(methylsulfonyl)phenyl]methanone. ¹H NMR (400 MHz, DMSO-d₆): δ 8.29-8.08 (dd, 1H), 7.79-7.71 (m, 1H), 7.69-7.66 (dd, 0.5H), 7.56-7.56 (d, 0.5H), 7.44-7.39 (m, 1.5H), 7.28-7.16 (dd, 1H), 7.04-7.01 (dd, 1H), 6.64-6.64 (m, 1H), 6.62-6.62 (d, 0.5H), 6.54-6.45 (dd, 1H), 5.00-4.33 (m, 2H), 4.29-3.55 (m, 4H), 3.48-3.42 (m, 5H), 3.39-3.35 (d, 3H), 2.68-2.00 (m, 2H), 1.97-1.87 (m, 2H), 1.73-1.63 (m, 2H), 1.60-1.51 (m, 2H), 1.48-1.39 (m, 2H), 1.11-0.95 (m, 3H). MS (EI) for C₂₉H₃₂FN₃O₄S: 538.2 (MH⁺).

5-(4-{[2-Ethyl-3-fluoro-4-(methylsulfonyl)phenyl]carbonyl}-2,3,4,5-tetrahydro-1,4-benzoxazepin-7-yl)-N,N-dimethylpyridin-2-amine. ¹H NMR (400 MHz, DMSO-d₆): δ 8.43-8.21 (m, 1H), 7.83-7.43 (m, 4H), 7.29-7.02 (m, 2H), 6.73-6.62 (m, 1H), 5.02-4.77 (m, 1H), 4.48-3.99 (m, 4H), 3.57 (m, 1H), 3.35 (m, 3H), 3.05 (d, 6H), 2.67 (m, 1H), 2.31-2.03 (m, 1H), 1.12-0.95 (m, 3H). MS (EI) for C₂₆H₂₈FN₃O₄S, found 498 (MH+).

[7-(6-Aminopyridin-3-yl)-2,3-dihydro-1,4-benzoxazepin-4(5H)-yl][2-ethyl-4-(ethylsulfonyl)-3-fluorophenyl]methanone. ¹H NMR (400 MHz, DMSO-d₆): δ 8.24-8.00 (dd, 1H), 7.74-7.68 (m, 1.5H), 7.57-7.57 (d, 0.5H), 7.44-7.38 (m, 1.5H), 7.29-7.12 (dd, 1H), 7.03-7.01 (dd, 1H), 6.58-6.58 (d, 0.5H), 6.53-6.45 (m, 1H), 6.06 (s, 2H), 5.00-4.35 (m, 2H), 4.27-3.53 (m, 4H), 3.45-3.38 (m, 2H), 2.69-2.03 (m, 2H), 1.17 (q, 3H), 1.11-0.96 (m, 3H). MS (EI) for C₂₅H₂₆FN₃O₄S: 484.2 (MH⁺).

[7-(6-Amino-1-oxidopyridin-3-yl)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.42-8.19 (dd, 1H), 7.77-7.71 (m, 1H), 7.68-7.67 (d, 0.5H), 7.51-7.47 (m, 1.5H), 7.29-7.20 (m, 1H), 7.12-7.10 (d, 0.5H), 7.05-7.02 (dd, 1H), 6.89 (s, 2H), 6.90-6.81 (dd, 1H), 6.79-6.78 (m, 0.5H), 5.01-4.36 (m, 2H), 4.32-3.56 (m, 4H), 3.42-3.35 (d, 3H), 2.68-2.02 (m, 2H), 1.11-0.96 (m, 3H). MS (EI) for C₂₄H₂₄FN₃O₅S: 486.2 (MH⁺).

[2-Ethyl-3-fluoro-4-(methylsulfonyl)phenyl][7-(6-{[2-(morpholin-4-yl)ethyl]amino}pyridin-3-yl)-2,3-dihydro-1,4-benzoxazepin-4(5H)-yl]methanone. ¹H NMR (400 MHz, DMSO-d₆): δ 8.30-8.09 (dd, 1H), 7.78-7.71 (m, 1H), 7.71-7.57 (m, 1H), 7.44-7.40 (m, 1.5H), 7.28-7.15 (dd, 1H), 7.04-7.01 (dd, 1H), 6.65-6.65 (d, 0.5H), 6.59-6.50 (m, 2H), 5.00-4.33 (m, 2H), 4.30-3.53 (m, 4H), 3.59 (t, 4H), 3.43-3.33 (m, 2H), 3.39-3.35 (d, 3H), 2.70-2.00 (m, 2H), 2.50-2.45 (m, 2H), 2.42 (bs, 4H), 1.11-0.96 (m, 3H). MS (EI) for C₃₀H₃₅FN₄O₅S: 583.3 (MH⁺).

[7-(6-Aminopyridazin-3-yl)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.00-8.00 (d, 0.5H), 7.84-7.80 (m, 1.5H), 7.76-7.71 (m, 1H), 7.53-7.27 (dd, 1H), 7.14-7.05 (dd, 1H), 7.09-7.06 (d, 1H), 6.87-6.78 (dd, 1H), 6.51-6.48 (d, 2H), 6.90-6.81 (dd, 1H), 6.79-6.78 (m, 0.5H), 5.02-4.38 (m, 2H), 4.34-3.55 (m, 4H), 3.40-3.36 (d, 3H), 2.68-2.02 (m, 2H), 1.11-0.95 (m, 3H). MS (EI) for C₂₃H₂₃FN₄O₄S: 471.2 (MH⁺).

[7-(6-Amino-5-methylpyridin-3-yl)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.13-7.92 (dd, 1H), 7.78-7.71 (m, 1H), 7.58 (s, 1H), 7.45-7.40 (m, 1H), 7.32-7.32 (d, 0.5H), 7.28-7.11 (dd, 1H), 7.04-7.00 (dd, 1H), 6.70-6.70 (d, 0.5H), 5.85-5.84 (d, 2H), 5.00-4.35 (m, 2H), 4.26-3.53 (m, 4H), 3.37-3.35 (d, 3H), 2.68-2.04 (m, 2H), 2.12-2.08 (d, 3H), 1.11-0.99 (m, 3H). MS (EI) for C₂₅H₂₆FN₃O₄S: 484.2 (MH⁺).

[7-(5-Aminopyrazin-2-yl)-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 8.51-8.26 (dd, 1H), 7.96-7.88 (dd, 1H), 7.94-7.94 (d, 0.5H), 7.79-7.71 (m, 2H), 7.28-7.10 (dd, 1H), 7.06-7.06 (d, 0.5H), 7.05-7.02 (dd, 1H), 6.54-6.54 (d, 2H), 5.00-4.36 (m, 2H), 4.29-3.53 (m, 4H), 3.38-3.35 (d, 3H), 2.68-2.03 (m, 2H), 2.12-2.08 (d, 3H), 1.11-0.97 (m, 3H). MS (EI) for C₂₃H₂₃FN₄O₄S: 471.2 (MH⁺).

[2-Ethyl-3-fluoro-4-(methylsulfonyl)phenyl][7-{6-[(2-hydroxyethyl)amino]pyridin-3-yl}-2,3-dihydro-1,4-benzoxazepin-4(5H)-yl]methanone. ¹H NMR (400 MHz, DMSO-d₆): δ 8.29-8.08 (m, 1H), 7.79-7.71 (m, 1H), 7.71-7.68 (dd, 0.5H), 7.57-7.57 (d, 0.5H), 7.45-7.40 (m, 1.5H), 7.29-7.16 (dd, 1H), 7.04-7.01 (dd, 1H), 6.67-6.64 (m, 1.5H), 6.60-6.51 (dd, 1H), 5.01-4.34 (m, 3H), 4.30-3.50 (m, 6H), 3.39-3.36 (d, 3H), 3.37-3.30 (m, 2H), 2.69-2.00 (m, 2H), 1.12-0.96 (m, 3H). MS (EI) for C₂₆H₂₈FN₃O₅S: 514.2 (MH⁺).

[7-(6-Aminopyridazin-3-yl)-2,3-dihydro-1,4-benzoxazepin-4(5H)-yl][3-fluoro-2-methyl-4-(methylsulfonyl)phenyl]methanone. ¹H NMR (400 MHz, DMSO-d₆): δ 8.00-8.00 (d, 0.5H), 7.91-7.89 (d, 0.5H), 7.84-7.80 (m, 1H), 7.75-7.71 (m, 1H), 7.55-7.53 (d, 0.5H), 7.29-7.27 (d, 0.5H), 7.18-7.16 (d, 0.5H), 7.10-7.07 (dd, 1H), 7.03-7.02 (d, 0.5H), 6.95-6.83 (dd, 1H), 6.74 (s, 1H), 6.64 (s, 1H), 4.96-4.35 (m, 2H), 4.35-3.56 (m, 4H), 3.38-3.34 (d, 3H), 2.11-1.75 (dd, 3H). MS (EI) for C₂₂H₂₁FN₄O₄S: 457.2 (MH⁺).

[7-(6-Aminopyridazin-3-yl)-2,3-dihydro-1,4-benzoxazepin-4(5H)-yl][2-ethyl-4-(ethylsulfonyl)-3-fluorophenyl]methanone. ¹H NMR (400 MHz, DMSO-d₆): δ 8.01-8.00 (d, 0.5H), 7.93-7.91 (d, 0.5H), 7.83-7.78 (m, 1H), 7.73-7.67 (m, 1H), 7.57-7.55 (d, 0.5H), 7.30-7.28 (d, 0.5H), 7.14-7.12 (d, 0.5H), 7.09-7.07 (d, 1H), 7.02-7.02 (d, 0.5H), 6.97-6.85 (dd, 1H), 6.81 (s, 1H), 6.66 (s, 1H), 5.02-4.38 (m, 2H), 4.36-3.58 (m, 4H), 3.47-3.35 (m, 2H), 2.67-2.01 (m, 2H), 1.20-1.14 (q, 3H), 1.10-0.94 (m, 3H). MS (EI) for C₂₄H₂₅FN₄O₄S: 485.2 (MH⁺).

[7-(3,4-Dihydro-2H-pyrido[3,2-b][1,4]oxazin-7-yl)-2,3-dihydro-1,4-bezoxazepin-4(5H)-yl][2-ethyl-3-fluoro-4-(methylsulfonyl)phenyl]methanone. ¹H NMR (400 MHz, DMSO-d₆): δ 7.92-7.91 (d, 0.5H), 7.76-7.71 (m, 1H), 7.71-7.70 (d, 0.5H), 7.60-7.59 (d, 0.5H), 7.45-7.40 (m, 1H), 7.28 (s, 0.5H), 7.28-7.26 (d, 0.5H), 7.15-7.13 (d, 0.5H), 7.07-7.06 (d, 0.5H), 7.03-7.00 (dd, 1H), 6.92 (s, 1H), 6.75-6.74 (d, 0.5H), 5.00-4.34 (m, 2H), 4.31-3.53 (m, 6H), 3.45-3.40 (m, 2H), 3.38-3.35 (d, 3H), 2.68-2.02 (m, 2H), 1.11-0.96 (m, 3H). MS (EI) for C₂₆H₂₆FN₃O₅S: 512.2 (MH⁺).

[7-(2-Amino-1,3-thiazol-5-yl)-2,3-dihydro-1,4-benzoxazepin-4(5H)-yl][3-fluoro-2-methyl-4-(methylsulfonyl)phenyl]methanone. ¹H NMR (400 MHz, DMSO-d₆): δ 7.76-7.70 (m, 1H), 7.41-7.40 (d, 0.5H), 7.34 (s, 0.5H), 7.29-7.27 (d, 0.5H), 7.29-7.22 (m, 1H), 7.17-7.15 (d, 0.5H), 7.12-7.09 (d, 2H), 7.09 (s, 0.5H), 6.98-6.95 (dd, 1H), 6.55-6.54 (dd, 0.5H), 4.88-4.27 (m, 2H), 4.25-3.53 (m, 4H), 3.38-3.34 (d, 3H), 2.12-1.80 (dd, 3H). MS (EI) for C₂₁H₂₀FN₃O₄S₂: 462.2 (MH⁺).

[7-(2-Amino-1,3-thiazol-5-yl)-2,3-dihydro-1,4-benzoxazepin-4(5H)-yl][2-ethyl-4-(ethylsulfonyl)-3-fluorophenyl]methanone. ¹H NMR (400 MHz, DMSO-d₆): δ 7.73-7.69 (dd, 1H), 7.41-7.41 (d, 0.5H), 7.35 (s, 0.5H), 7.29-7.27 (d, 0.5H), 7.29-7.22 (m, 1H), 7.13-7.10 (m, 2.5H), 7.07 (s, 0.5H), 6.98-6.95 (dd, 1H), 6.52-6.52 (d, 0.5H), 4.95-4.33 (m, 2H), 4.27-3.52 (m, 4H), 3.47-3.40 (m, 2H), 2.69-2.07 (m, 2H), 1.23-1.15 (m, 3H), 1.11-0.98 (m, 3H). MS (EI) for C₂₃H₂₄FN₃O₄S₂: 490.1 (MH⁺).

N-[5-(4-{[2-Ethyl-3-fluoro-4-(methylsulfonyl)-phenyl]carbonyl}-2,3,4,5-tetrahydro-1,4-benzoxazepin-7-yl)-1,3-thiazol-2-yl]-N²-methylglycinamide. ¹H NMR (400 MHz, DMSO-d₆): δ 7.83 (s, 0.5H), 7.75-7.71 (m, 1H), 7.63-7.62 (d, 0.5H), 7.61 (s, 0.5H), 7.49-7.45 (m, 1H), 7.29-7.10 (dd, 1H), 7.04-7.01 (dd, 1H), 6.77-6.76 (d, 0.5H), 5.00-4.35 (m, 2H), 4.30-3.54 (m, 4H), 3.42-3.47 (m, 2H), 3.40-3.35 (d, 3H), 2.68-2.03 (m, 2H), 1.30-1.11 (dd, 1H), 1.11-0.97 (m, 3H). MS (EI) for C₂₅H₂₇FN₄O₅S₂: 547.2 (MH⁺).

N-[5-(4-{[2-Ethyl-3-fluoro-4-(methylsulfonyl)-phenyl]carbonyl}-2,3,4,5-tetrahydro-1,4-benzoxazepin-7-yl)-1,3-thiazol-2-yl]glycinamide. ¹H NMR (400 MHz, DMSO-d₆): δ 7.82 (s, 0.5H), 7.75-7.71 (m, 1H), 7.62-7.62 (d, 0.5H), 7.60 (s, 0.5H), 7.49-7.45 (m, 1H), 7.29-7.11 (dd, 1H), 7.04-7.01 (dd, 1H), 6.76-6.75 (d, 0.5H), 5.57 (bs, 2H), 5.00-4.35 (m, 2H), 4.32-3.52 (m, 4H), 3.41-3.9 (d, 2H), 3.41-3.35 (d, 3H), 2.68-2.02 (m, 2H), 1.11-0.97 (m, 3H). MS (EI) for C₂₄H₂₅FN₄O₅S₂: 533.2 (MH⁺).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]glycinamide. ¹H NMR (400 MHz, DMSO-d₆): δ 8.66-8.43 (dd, 1H), 8.22-8.11 (m, 1.5H), 7.89-7.86 (dd, 0.5H), 7.77-7.72 (m, 1.5H), 7.61-7.55 (m, 1H), 7.29-7.11 (dd, 1H), 7.11-7.07 (dd, 1H), 6.87-6.86 (d, 0.5H), 5.04-4.40 (m, 2H), 4.33-3.57 (m, 4H), 3.36-3.36 (d, 3H), 3.33 (bs, 2H), 3.33-3.32 (d, 2H), 2.68-2.07 (m, 2H), 1.12-0.98 (m, 3H). MS (EI) for C₂₈H₃ᵢFN₄O₅S: 555.2 (MH⁺).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]formamide. ¹H NMR (400 MHz, DMSO-d₆): δ 10.74 (s, 1H), 10.72-10.66 (dd, 0.5H), 9.34-9.27 (dd, 0.5H), 8.66-8.58 (dd, 0.5H), 8.44-8.34 (m, 1H), 8.14-8.06 (m, 1H), 7.88-7.78 (m, 0.5H), 7.77-7.72 (m, 1H), 7.60-7.53 (m, 1H), 7.29-7.27 (d, 0.5H), 7.149-7.13 (d, 0.5H), 7.11-7.08 (dd, 1H), 7.03-6.95 (dd, 0.5H), 6.86-6.82 (d, 0.5H), 5.04-4.40 (m, 2H), 4.30-3.57 (m, 4H), 3.37-3.35 (d, 3H), 2.68-2.04 (m, 2H), 1.11-0.97 (d, 3H). MS (EI) for C₂₅H₂₄FN₃O₅S: 498.2 (MH⁺).

N-[5-(4-{[2-Ethyl-3-fluoro-4-(methylsulfonyl)-phenyl]carbonyl}-2,3,4,5-tetrahydro-1,4-benzoxazepin-7-yl)-1,3-thiazol-2-yl]-L-alanamide. ¹H NMR (400 MHz, DMSO-d₆): δ 7.82 (s, 0.5H), 7.75-7.72 (t, 1H), 7.62-7.62 (d, 0.5H), 7.60 (s, 0.5H), 7.48-7.44 (m, 1H), 7.29-7.27 (d, 0.5H), 7.13-7.11 (d, 0.5H), 7.04-7.01 (dd, 1H), 6.75-6.74 (d, 0.5H), 5.69 (bs, 2H), 5.00-4.34 (m, 2H), 4.31-3.56 (m, 5H), 3.41-3.35 (d, 3H), 2.68-2.02 (m, 2H), 1.25-1.22 (dd, 3H), 1.12-0.96 (m, 3H). MS (EI) for C₂₅H₂₇FN₄O₅S₂: 547.2 (MH⁺).

N-[5-(4-{[2-Ethyl-3-fluoro-4-(methylsulfonyl)-phenyl]carbonyl}-2,3,4,5-tetrahydro-1,4-benzoxazepin-7-yl)-1,3-thiazol-2-yl]-D-alanamide. ¹H NMR (400 MHz, DMSO-d₆): δ 7.82 (s, 0.5H), 7.75-7.72 (t, 1H), 7.62-7.62 (d, 0.5H), 7.60 (s, 0.5H), 7.48-7.44 (m, 1H), 7.29-7.27 (d, 0.5H), 7.13-7.11 (d, 0.5H), 7.04-7.01 (dd, 1H), 6.75-6.74 (d, 0.5H), 5.69 (bs, 2H), 5.00-4.34 (m, 2H), 4.31-3.56 (m, 5H), 3.41-3.35 (d, 3H), 2.68-2.02 (m, 2H), 1.25-1.22 (dd, 3H), 1.12-0.96 (m, 3H). MS (EI) for C₂₅H₂₇FN₄O₅S₂: 547.2 (MH⁺).

1-Amino-N-[5-(4-{[2-ethyl-3-fluoro-4-(methylsulfonyl)-phenyl]carbonyl}-2,3,4,5-tetrahydro-1,4-benzoxazepin-7-yl)pyridin-2-yl]cyclopropane-carboxamide. ¹H NMR (400 MHz, DMSO-d₆): δ 10.47 (bs, 1H), 8.66-8.66 (dd, 0.5H), 8.44-8.44 (dd, 0.5H), 8.13-8.11 (m, 1.5H), 7.87-7.84 (dd, 0.5H), 7.77-7.72 (m, 1.5H), 7.61-7.56 (m, 1H), 7.29-7.27 (d, 0.5H), 7.14-7.12 (d, 0.5H), 7.11-7.07 (dd, 1H), 6.87-6.86 (d, 0.5H), 5.04-4.40 (m, 2H), 4.33-3.57 (m, 4H), 3.37-3.36 (d, 3H), 2.76 (bs, 2H), 2.70-2.04 (m, 2H), 1.26-0.96 (m, 7H). MS (EI) for C₂₈H₂₉FN₄O₅S: 553.2 (MH⁺).

1-Amino-N-[5-(4-{[2-ethyl-3-fluoro-4-(methylsulfonyl)-phenyl]carbonyl}-2,3,4,5-tetrahydro-1,4-benzoxazepin-7-yl)pyridin-2-yl]cyclobutane-carboxamide. ¹H NMR (400 MHz, DMSO-d₆): δ 8.65-8.64 (d, 0.5H), 8.44-8.43 (d, 0.5H), 8.25-8.13 (m, 1.5H), 7.89-7.86 (dd, 0.5H), 7.77-7.72 (m, 1.5H), 7.61-7.56 (m, 1H), 7.29-7.27 (d, 0.5H), 7.14-7.12 (d, 0.5H), 7.10-7.07 (dd, 1H), 6.87-6.86 (d, 0.5H), 5.04-4.40 (m, 2H), 4.33-3.55 (m, 4H), 3.37-3.36 (d, 3H), 2.76 (bs, 2H), 2.69-1.78 (m, 8H), 1.11-0.98 (m, 3H). MS (EI) for C₂₉H₃ᵢFN₄O₅S: 567.3 (MH⁺).

1-[5-(4-{[2-Ethyl-3-fluoro-4-(methylsulfonyl)phenyl]carbonyl}-2,3,4,5-tetrahydro-1,4-benzoxazepin-7-yl)pyridin-2-yl]guanidine. ¹H NMR (400 MHz, DMSO-d₆): δ 8.51-8.51 (d, 0.5H), 8.25-8.25 (d, 0.5H), 8.12 (bs, 3H), 8.03-8.00 (dd, 0.5H), 7.79-7.72 (m, 1.5H), 7.69-7.69 (d, 0.5H), 7.55-7.50 (m, 1H), 7.29-7.27 (d, 0.5H), 7.16-7.14 (d, 0.5H), 7.09-7.07 (dd, 1.0H), 7.02-7.00 (d, 0.5H), 6.92-6.90 (d, 0.5H), 6.77-6.77 (d, 0.5H), 5.03-4.38 (m, 2H), 4.34-3.54 (m, 4H), 3.37-3.36 (d, 3H), 2.68-2.03 (m, 2H), 1.12-0.96 (m, 3H). MS (EI) for C₂₅H₂₆FN₅O₄S: 512.2 (MH⁺).

[2-Ethyl-3-fluoro-4-(methylsulfonyl)phenyl]{7-[6-(pyrimidin-2-ylamino)pyridin-3-yl]-2,3-dihydro-1,4-benzoxazepin-4(5H)-yl}methanone. ¹H NMR (400 MHz, DMSO-d₆): δ 9.02-9.00 (d, 1H), 8.99-8.98 (d, 1H), 8.21-8.08 (m, 1H), 7.99 (bs, 0.5H), 7.92 (s, 0.5H), 7.76-7.71 (m, 1H), 7.64-7.59 (m, 1.5H), 7.51-7.48 (m, 0.5H), 7.44-7.38 (m, 1H), 7.28-7.26 (d, 0.5H), 7.18-7.16 (m, 0.5H), 7.12-7.10 (d, 0.5H), 7.05-7.03 (d, 1.0H), 6.72-6.71 (d, 0.5H), 6.69-6.57 (dd, 1H), 4.99-4.40 (m, 2H), 4.25-3.56 (m, 4H), 3.31-3.30 (d, 3H), 2.68-2.09 (m, 2H), 1.11-0.98 (m, 3H). MS (EI) for C₂₈H₂₆FN₅O₄S: 548.2 (MH⁺).

[2-Ethyl-3-fluoro-4-(methylsulfonyl)phenyl]{7-[2-(1H-pyrazol-3-ylamino)-1,3-thiazol-5-yl]-2,3-dihydro-1,4-benzoxazepin-4(5H)-yl}methanone. ¹H NMR (400 MHz, DMSO-d₆): δ 8.14-8.11 (dd, 1H), 7.87 (s, 0.5H), 7.78-7.72 (m, 1H), 7.67-7.67 (d, 0.5H), 7.64 (s, 0.5H), 7.50-7.46 (m, 1H), 7.30-7.28 (d, 0.5H), 7.15-7.13 (d, 0.5H), 7.04 (t, 1H), 6.79-6.78 (d, 0.5H), 5.89-5.87 (dd, 1H), 5.60 (s, 1H), 5.57 (s, 1H), 5.01-4.35 (m, 2H), 4.33-3.56 (m, 4H), 3.47-3.36 (d, 3H), 2.68-2.01 (m, 2H), 1.11-0.97 (m, 3H). MS (EI) for C₂₅H₂₄FN₅O₄S₂: 542.2 (MH⁺).

5-(4-{[2-Ethyl-3-fluoro-4-(methylsulfonyl)phenyl]carbonyl}-2,3,4,5-tetrahydro-1,4-benzoxazepin-7-yl)-3-fluoropyridin-2-amine. ¹H NMR (400 MHz, DMSO-d₆): δ 8.13 (bs, 0.5H), 7.92 (bs, 0.5H), 7.76-7.71 (m, 1.5H), 7.66-7.65 (d, 0.5H), 7.50-7.46 (m, 1.5H), 7.28-7.26 (d, 0.5H), 7.13-7.11 (d, 0.5H), 7.04-7.01 (dd, 1H), 6.81-6.80 (d, 0.5H), 6.33 (s, 2H), 5.00-4.36 (m, 2H), 4.29-3.55 (m, 4H), 3.36-3.32 (d, 3H), 2.68-2.05 (m, 2H), 1.11-0.98 (d, 3H). MS (EI) for C₂₄H₂₃F₂N₃O₄S: 488.1 (MH⁺).

2-Amino-5-(4-{[2-ethyl-3-fluoro-4-(methylsulfonyl)-henyl]carbonyl}-2,3,4,5-tetrahydro-1,4-benzoxazepin-7-yl)pyridine-3-carbonitrile. ¹H NMR (400 MHz, DMSO-d₆): δ 8.58-8.57 (d, 0.5H), 8.35-8.35 (d, 0.5H), 8.24-8.23 (d, 0.5H), 8.02-8.02 (d, 0.5H), 7.75-7.71 (m, 1.5H), 7.54-7.50 (m, 1H), 7.29-7.27 (d, 0.5H), 7.11-7.09 (d, 0.5H), 7.06-7.03 (dd, 1H), 7.03 (s, 2H), 6.89-6.88 (d, 0.5H), 5.01-4.38 (m, 2H), 4.31-3.54 (m, 4H), 3.36-3.36 (d, 3H), 2.68-2.06 (m, 2H), 1.12-0.98 (d, 3H). MS (EI) for C₂₅H₂₃FN₄O₄S: 495.1 (MH⁺).

2-Amino-5-(4-{[2-ethyl-3-fluoro-4-(methylsulfonyl)-phenyl]carbonyl}-2,3,4,5-tetrahydro-1,4-benzoxazepin-7-yl)-N-methylpyridine-3-carboxamide. ¹H NMR (400 MHz, DMSO-d₆): δ 8.63-8.60 (q, 0.5H), 8.50-8.46 (q, 0.5H), 8.43-8.42 (d, 0.5H), 8.18-8.16 (dd, 1H), 8.02-8.01 (d, 0.5H), 7.78-7.72 (m, 1H), 7.71-7.70 (d, 0.5H), 7.56-7.52 (m, 1H), 7.29-7.27 (d, 0.5H), 7.18 (bs, 2H), 7.14-7.12 (d, 0.5H), 7.09-7.05 (dd, 1H), 6.88-6.87 (d, 0.5H), 5.02-4.82 (dd, 1H), 4.43 (bs, 1H), 4.26-3.57 (m, 4H), 3.36-3.35 (d, 3H), 2.80-2.77 (dd, 3H), 2.68-2.12 (m, 2H), 1.12-1.10 (d, 3H). MS (EI) for C₂₅H₂₃FN₄O₄S: 495.1 (MH⁺).

2-Amino-5-(4-{[2-ethyl-3-fluoro-4-(methylsulfonyl)-phenyl]carbonyl}-2,3,4,5-tetrahydro-1,4-benzoxazepin-7-yl)-N,N-dimethylpyridine-3-carboxamide. ¹H NMR (400 MHz, DMSO-d₆): δ 8.63-8.35 (d, 0.5H), 8.16-8.16 (d, 0.5H), 7.75-7.70 (m, 1.5H), 7.65-7.64 (d, 0.5H), 7.50-7.46 (m, 1.5H), 7.28-7.26 (d, 0.5H), 7.11-7.09 (d, 0.5H), 7.05-7.01 (dd, 1H), 6.83-6.83 (d, 0.5H), 6.10-6.09 (d, 2H), 5.01-4.78 (dd, 1H), 4.42 (bs, 1H), 4.25-3.55 (m, 4H), 3.35-3.34 (d, 3H), 2.96 (bs, 6H), 2.68-2.11 (m, 2H), 1.11-0.99 (d, 3H). MS (EI) for C₂₇H₂₉FN₄O₅S: 541.2 (MH⁺).

4-{[2-Ethyl-3-fluoro-4-(methylsulfonyl)phenyl]carbonyl}-7-(1H-pyrazolo[3,4-b]pyridin-5-yl)-2,3,4,5-tetrahydro-1,4-benzoxazepine. ¹H NMR (400 MHz, DMSO-d₆): δ 13.72 (s, 1H), 8.85-8.85 (d, 0.5H), 8.65-8.64 (d, 0.5H), 8.49-8.48 (d, 0.5H), 8.20-8.15 (dd, 1H), 8.19-8.18 (d, 0.5H), 7.81-7.72 (m, 1.5H), 7.65-7.61 (m, 1H), 7.30-7.28 (d, 0.5H), 7.15-7.13 (d, 0.5H), 7.13-7.11 (dd, 1H), 6.89-6.89 (d, 0.5H), 5.06-4.41 (m, 2H), 4.34-3.56 (m, 4H), 3.38-3.36 (d, 3H), 2.69-2.08 (m, 2H), 1.12-0.99 (d, 3H). MS (EI) for C₂₅H₂₃FN₄O₄S: 495.2 (MH⁺).

4-{[2-Ethyl-3-fluoro-4-(methylsulfonyl)phenyl]carbonyl}-7-(1H-pyrrolo[2,3-b]pyridin-5-yl)-2,3,4,5-tetrahydro-1,4-benzoxazepine. ¹H NMR (400 MHz, DMSO-d₆): δ 11.71 (s, 1H), 8.52-8.21 (m, 1H), 7.93-7.50 (m, 4H), 7.30-7.09 (m, 2H), 6.84-6.49 (m, 2H), 5.04-4.84 (m, 1H), 4.43-4.10 (m, 4H), 3.58 (m, 1H), 3.37 (m, 3H), 2.66 (m, 1H), 1.20 (m, 1H), 1.10-1.00 (m, 3H). MS (EI) for C₂₉H₃₃FN₃O₄S, found 539 (MH+).

4-{[2-Ethyl-3-fluoro-4-(methylsulfonyl)phenyl]carbonyl}-7-phenyl-2,3,4,5-tetrahydro-1,4-benzoxazepine. ¹H NMR (400 MHz, DMSO-d₆): δ 7.76-7.68 (m, 2H), 7.54-7.30 (m, 6H), 7.16-6.75 (m, 2H), 5.04-4.82 (m, 1H), 4.44-4.02 (m, 4H), 3.58 (m, 1H), 3.36 (m, 3H), 2.65 (m, 1H), 2.65 (m, 1H), 2.32-2.04 (m, 1H), 1.09-0.98 (m, 3H). MS (EI) for C₂₅H₂₄FNO₄S, found 454 (MH+).

1-(4-{[7-(1,3-Benzodioxol-5-yl)-2,3-dihydro-1,4-benzoxazepin-4(5H)-yl]carbonyl}phenyl)ethanol. ¹H NMR (400 MHz, DMSO-d₆): δ 7.59-6.72 (m, 10H), 6.05 (s, 2H), 5.25 (m, 1H), 4.78 (m, 2H), 4.52 (m, 1H), 4.24 (m, 1H), 4.13 (m, 1H), 3.98 (m, 1H), 3.76 (m, 1H), 1.34 (d, 3H). MS (EI) for C₂₅H₂₃NO₅, found 418.2 (MH+).

4-{[7-(1,3-Benzodioxol-5-yl)-2,3-dihydro-1,4-benzoxazepin-4(5H)-yl]carbonyl}phenyl)methanol. ¹H NMR (400 MHz, DMSO-d₆): δ 7.59-6.80 (m, 10H), 6.06 (s, 2H), 5.30 (m, 1H), 4.81 (m, 1H), 4.54 (m, 3H), 4.26 (m, 1H), 4.13 (m, 1H), 3.98 (m, 1H), 3.76 (m, 1H). MS (EI) for C₂₄H₂₁NO₅, found 404.2 (MH+).

4-{[2-Ethyl-3-fluoro-4-(methylsulfonyl)phenyl]carbonyl}-7-[4-(1H-imidazol-2-yl)-3-methylphenyl]-2,3,4,5-tetrahydro-1,4-benzoxazepine. ¹H NMR (400 MHz, DMSO-d₆): δ 12.28-12.25 (d, 1H), 7.80-7.72 (m, 1.5H), 7.69-7.67 (d, 0.5H), 7.62-7.57 (m, 2.5H), 7.39 (bs, 0.5H), 7.36-7.34 (d, 0.5H), 7.30-7.28 (d, 0.5H), 7.25 (bs, 1H), 7.15-7.13 (d, 0.5H), 7.10-7.07 (m, 2H), 6.88-6.87 (d, 0.5H), 5.05-4.40 (m, 2H), 4.32-3.55 (m, 4H), 3.36-3.34 (d, 3H), 2.63-2.57 (d, 3H), 2.69-2.09 (m, 2H), 1.12-0.99 (m, 3H). MS (EI) for C₂₉H₂₈FN₃O₄S: 534.2 (MH⁺).

4-{[2-Ethyl-3-fluoro-4-(methylsulfonyl)phenyl]carbonyl}-7-[3-fluoro-4-(1H-imidazol-2-yl)phenyl]-2,3,4,5-tetrahydro-1,4-benzoxazepine. ¹H NMR (400 MHz, DMSO-d₆): δ 12.25-12.23 (d, 1H), 8.11-8.00 (m, 1H), 8.85-8.84 (d, 0.5H), 7.79-7.64 (d, 3H), 7.49-7.46 (d, 0.5H), 7.40-7.38 (dd, 0.5H), 7.31-7.28 (m, 1.5H), 7.14-7.08 (m, 1.5H), 6.95-6.95 (d, 0.5H), 5.06-4.42 (m, 2H), 4.35-3.56 (m, 4H), 3.39-3.36 (d, 3H), 2.69-2.06 (m, 2H), 1.12-0.99 (m, 3H). MS (EI) for C₂₈H₂₅F₂N₃O₄S: 538.2 (MH⁺).

4-{[2-Ethyl-3-fluoro-4-(methylsulfonyl)phenyl]carbonyl}-7-[2-fluoro-4-(1H-imidazol-2-yl)phenyl]-2,3,4,5-tetrahydro-1,4-benzoxazepine. ¹H NMR (400 MHz, DMSO-d₆): δ 12.67 (s, 1H), 7.89-7.72 (m, 3H), 7.65-7.61 (t, 0.5H), 7.65 (s, 0.5H), 7.50-7.44 (m, 1H), 7.41-7.37 (t, 0.5H), 7.38-7.29 (m, 1.5H), 7.15-7.06 (m, 2.5H), 6.76-6.76 (t, 0.5H), 5.04-4.41 (m, 2H), 4.38-3.58 (m, 4H), 3.36-3.35 (d, 3H), 2.69-2.10 (m, 2H), 1.12-0.99 (m, 3H). MS (EI) for C₂₈H₂₅F₂N₃O₄S: 538.2 (MH⁺).

7-[4-(1H-Imidazol-2-yl)phenyl]-4-{[4-(methylsulfonyl-phenyl]carbonyl}-2,3,4,5-tetrahydro-1,4-benzoxazepine. ¹H NMR (400 MHz, DMSO-d₆): δ 12.57 (s, 1H), 8.03-7.96 (m, 4H), 7.77-7.66 (m, 3H), 7.61-7.49 (m, 3H), 7.27 (bs, 1H), 7.11-6.91 (m, 2H), 4.89-4.52 (d, 2H), 4.30-4.16 (m, 2H), 4.05-3.71 (m, 2H), 3.28-3.26 (d, 3H). MS (EI) for C₂₆H₂₃N₃O₄S: 474.2 (MH⁺).

7-[4-(4,5-Dihydro-1H-imidazol-2-yl)-2-fluorophenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 7.77-7.60 (m, 4H), 7.50-7.42 (m, 1H), 7.39-7.35 (t, 0.5H), 7.30-7.28 (d, 0.5H), 7.19 (bs, 1H), 7.14-7.10 (m, 1.5H), 6.76-6.75 (t, 0.5H), 5.03-4.41 (m, 2H), 4.35-3.59 (m, 8H), 3.35-3.33 (d, 3H), 2.72-2.07 (m, 2H), 1.12-0.98 (m, 3H). MS (EI) for C₂₈H₂₇F₂N₃O₄S: 540.2 (MH⁺).

2-[4-(4-{[2-Ethyl-3-fluoro-4-(methylsulfonyl)phenyl]carbonyl}-2,3,4,5-tetrahydro-1,4-benzoxazepin-7-yl)phenyl]-1H-imidazol-1-ol. ¹H NMR (400 MHz, DMSO-d₆): δ 8.22-7.94 (m, 2H), 7.83-7.70 (m, 3H), 7.60-7.46 (m, 2H), 7.30-7.28 (d, 0.5H), 7.22 (bs, 1H), 7.16-7.14 (m, 0.5H), 7.10-7.06 (m, 1H), 6.84-6.81 (m, 1H), 5.05-4.39 (m, 2H), 4.34-3.57 (m, 4H), 3.39-3.36 (d, 3H), 2.69-2.04 (m, 2H), 1.12-0.97 (m, 3H). MS (EI) for C₂₈H₂₆FN₃O₅S: 540.2 (MH⁺).

4-{[2-Ethyl-3-fluoro-4-(methylsulfonyl)phenyl]carbonyl}-7-(2-fluorophenyl)-2,3,4,5-tetrahydro-1,4-benzoxazepine. ¹H NMR (400 MHz, CDCl₃): δ 7.89-7.80 (m, 1H), 7.62 (t, 0.3H), 7.48-7.08 (m, 7H), 6.67-6.67 (t, 0.7H), 5.09-4.74 (dd, 0.6H), 4.45-3.97 (m, 4.8H), 3.65-3.61 (m, 0.6H), 3.24-3.23 (d, 3H), 2.82-2.29 (m, 2H), 1.22-1.13 (m, 3H). MS (EI) for C₂₅H₂₃F₂NO₄S: 472.1 (MH⁺).

4-{[2-Ethyl-3-fluoro-4-(methylsulfonyl)phenyl]carbonyl}-7-(3-fluorophenyl)-2,3,4,5-tetrahydro-1,4-benzoxazepine. ¹H NMR (400 MHz, CDCl₃): δ 7.89-7.80 (m, 1H), 7.64-7.64 (d, 0.4H), 7.48-7.34 (m, 2.6H), 7.31-7.27 (m, 0.4H), 7.16-7.00 (m, 4H), 6.60-6.59 (d, 0.6H), 5.09-4.74 (dd, 0.8H), 4.44-3.97 (m, 4.4H), 3.68-3.59 (m, 0.8H), 3.25-3.24 (d, 3H), 2.82-2.22 (m, 2H), 1.22-1.12 (m, 3H). MS (EI) for C₂₅H₂₃F₂NO₄S: 472.1 (MH⁺).

5-[4-(4-{[2-Ethyl-3-fluoro-4-(methylsulfonyl)phenyl]carbonyl}-2,3,4,5-tetrahydro-1,4-benzoxazepin-7-yl)phenyl]isoxazol-3-amine. ¹H NMR (400 MHz, DMSO-d₆): δ 7.86-7.72 (m, 4H), 7.63-7.54 (m, 2H), 7.30-6.83 (m, 2H), 6.37-6.34 (d, 1H), 5.69 (s, 1H), 5.07-4.82 (m, 1H), 4.46-4.14 (m, 4H), 3.38 (s, 3H), 1.12-0.97 (m, 3H). MS (EI) for C₂₈H₂₆FN₃O₅S, found 536 (MH+).

1-(4-{[7-(2,3-Dihydro-1,4-benzodioxin-6-yl)-2,3-dihydro-1,4-benzoxazepin-4(5H)-yl]carbonyl}phenyl)-2,2,2-trifluoroethanone. ¹H NMR (400 MHz, DMSO-d₆): δ 8.10-6.84 (m, 10H), 4.85-0.71 (m, 10H). MS (EI) for C₂₆H₂₀F₃NO₅, found 502 (MH+19).

4-Chloro-2-fluoro-5-[(7-quinolin-3-yl-2,3-dihydro-1,4-benzoxazepin-4(5H)-yl)carbonyl]benzenesulfonamide. ¹H NMR (400 MHz, DMSO-d₆): δ 9.28-8.40 (m, 2H), 8.07-8.02 (m, 2H), 7.95-7.16 (m, 9H), 4.96 (m, 1H), 4.54 (s, 1H), 4.31 (m, 1H), 4.23-4.04 (m, 2H), 3.64 (m, 1H). MS (EI) for C₂₅H₁₉ClFN₃O₄S, found 512 (MH+).

4-{[4-(Methylsulfonyl)phenyl]carbonyl}-7-quinolin-3-yl-2,3,4,5-tetrahydro-1,4-benzoxazepine. ¹H NMR (400 MHz, DMSO-d₆): δ 9.28-8.40 (m, 2H), 8.09-7.14 (m, 11H), 4.95-3.74 (m, 6H), 3.30 (s, 3H). MS (EI) for C₂₆H₂₂N₂O₄S, found 549 (MH+).

4-[(7-Quinolin-3-yl-2,3-dihydro-1,4-benzoxazepin-4(5H)-yl)carbonyl]benzenesulfonamide. ¹H NMR (400 MHz, DMSO-d₆): δ 9.27-8.40 (m, 2H), 8.08-8.05 (m, 2H), 7.94-7.14 (m, 11H), 4.94-3.75 (m, 6H). MS (EI) for C₂₅H₂₁N₃O₄S, found 460 (MH+).

7-(1,3-Benzodioxol-5-yl)-4-{[4-(methylsulfonyl)-phenyl]carbonyl}-2,3,4,5-tetrahydro-1,4-benzoxazepine. ¹H NMR (400 MHz, DMSO-d₆): δ 7.99-7.97 (d, 2H), 7.66-7.12 (m, 5H), 7.05-6.78 (m, 3H), 6.06-6.04 (m, 2H), 4.85-3.70 (m, 6H), 3.26 (s, 3H). MS (EI) for C₂₄H₂₁NO₆S, found 452 (MH+).

4-{[7-(1,3-Benzodioxol-5-yl)-2,3-dihydro-1,4-benzoxazepin-4(5H)-yl]carbonyl}benzenesulfonamide. ¹H NMR (400 MHz, DMSO-d₆): δ 7.87-7.85 (d, 2H), 7.61-7.56 (m, 3H), 7.46-6.83 (m, 8H), 6.06-6.04 (m, 2H), 4.84-3.71 (m, 6H). MS (EI) for C₂₃H₂₀N₂O₆S, found 453 (MH+).

4-{[3-(Methylsulfonyl)phenyl]carbonyl}-7-quinolin-3-yl-2,3,4,5-tetrahydro-1,4-benzoxazepine. ¹H NMR (400 MHz, DMSO-d₆): δ 9.27-8.49 (m, 2H), 8.07-7.14 (m, 11H), 4.95-3.79 (m, 6H), 3.28-3.12 (m, 3H). MS (EI) for C₂₆H₂₂N₂O₄S, found 459 (MH+).

2-Chloro-5-[(7-quinolin-3-yl-2,3-dihydro-1,4-benzoxazepin-4(5H)-yl)carbonyl]benzenesulfonamide. ¹H NMR (400 MHz, DMSO-d₆): δ 9.27-8.49 (m, 2H), 8.07-7.14 (m, 12H), 4.94-3.80 (m, 6H). MS (EI) for C₂₅H₂₀ClN₃O₄S, found 494 (MH+).

4-Chloro-3-[(7-quinolin-3-yl-2,3-dihydro-1,4-benzoxazepin-4(5H)-yl)carbonyl]benzenesulfonyl fluoride. ¹H NMR (400 MHz, DMSO-d₆): δ 9.29-9.04 (m, 1H), 8.67-8.38 (m, 1H), 8.29-7.16 (m, 10H), 4.97-3.63 (m, 6H). MS (EI) for C₂₅H₁₈ClFN₂O₄S, found 497 (MH+).

N-Methyl-2-oxo-2-{4-[(7-quinolin-3-yl-2,3-dihydro-1,4-benzoxazepin-4(5H)-yl)carbonyl]phenyl}acetamide. ¹H NMR (400 MHz, DMSO-d₆): δ 9.28-9.11 (m, 1H), 8.92-8.91 (m, 1H), 8.67-8.40 (m, 1H), 8.13-7.15 (m, 11H), 4.94-3.75 (m, 6H), 2.79-2.78 (d, 3H). MS (EI) for C₂₈H₂₃N₃O₄, found 466 (MH+).

N,N-Dimethyl-2-oxo-2-{4-[(7-quinolin-3-yl-2,3-dihydro-1,4-benzoxazepin-4(5H)-yl)carbonyl]phenyl}acetamide. ¹H NMR (400 MHz, DMSO-d₆): δ 9.27-9.08 (m, 1H), 8.66-8.41 (m, 1H), 8.09-7.14 (m, 11H), 4.95-3.75 (m, 6H), 3.02-2.98 (m, 3H), 2.89-2.85 (m, 3H). MS (EI) for C₂₉H₂₅N₃O₄, found 480 (MH+).

3-[(7-Quinolin-3-yl-2,3-dihydro-1,4-benzoxazepin-4(5H)-yl)carbonyl]benzenesulfonamide. ¹H NMR (400 MHz, DMSO-d₆): δ 9.27-9.12 (m, 1H), 8.66-8.50 (m, 1H), 8.06-7.14 (m, 13H), 4.95-3.79 (m, 6H). MS (EI) for C₂₅H₂₁N₃O₄S, found 458 (MH−).

2-Fluoro-5-[(7-quinolin-3-yl-2,3-dihydro-1,4-benzoxazepin-4(5H)-yl)carbonyl]benzenesulfonamide. ¹H NMR (400 MHz, DMSO-d₆): δ 9.27-9.15 (m, 1H), 8.66-8.52 (m, 1H), 8.06-7.16 (m, 12H), 4.93-3.81 (m, 6H). MS (EI) for C₂₅H₂₀FN₃O₄S, found 478 (MH+).

4-Chloro-3-[(7-quinolin-3-yl-2,3-dihydro-1,4-benzoxazepin-4(5H)-yl)carbonyl]benzenesulfonamide. ¹H NMR (400 MHz, DMSO-d₆): δ 9.28-9.03 (m, 1H), 8.67-8.39 (m, 1H), 8.09-8.04 (m, 2H), 7.96-7.93 (m, 1H), 7.87-7.01 (m, 9H), 5.07-3.60 (m, 6H). MS (EI) for C₂₅H₂₀ClN₃O₄S, found 494 (MH+).

4-[(5-Bromo-2-thienyl)carbonyl]-7-(2-methyl-1H-benzimidazol-5-yl)-2,3,4,5-tetrahydro-1,4-benzoxazepine. ¹H NMR (400 MHz, DMSO-d₆): δ 12.10 (br, 1H), 7.62 (s, 1H), 7.51 (d, 2H), 7.41 (s, 1H), 7.01 (d, 2H), 6.25 (s, 2H), 4.98 (s, 2H), 4.30 (brs, 2H), 4.0 (brs, 2H), 2.50 (s, 3H). MS (EI) for C₂₂H₁₈BrN₃O₂S: 469.8 (MH⁺).

N-[4-(4-{[2-Ethyl-3-fluoro-4-(methylsulfonyl)-phenyl]carbonyl}-2,3,4,5-tetrahydro-1,4-benzoxazepin-7-yl)phenyl]-2,2,2-trifluoroacetamide. ¹H NMR (400 MHz, CDCl₃): δ 8.06-8.05 (d, 1H), 7.88-7.80 (m, 1H), 7.67-7.60 (m, 3.4H), 7.48-7.44 (m, 1H), 7.41-7.37 (m, 1H), 7.16-7.11 (dd, 1H), 7.09-7.06 (dd, 1H), 6.54-6.54 (d, 0.6H), 5.10-4.73 (dd, 0.8H), 4.43-3.96 (m, 4.4H), 3.68-3.59 (m, 0.8H), 3.26-3.24 (d, 3H), 2.82-2.21 (m, 2H), 1.22-1.12 (m, 3H). MS (EI) for C₂₇H₂₄F₄N₂O₅S: 563.1 (MH+).

7-(1H-Benzimidazol-6-yl)-4-{[4-(ethylsulfonyl)-3-fluoro-2-methylphenyl]carbonyl}-2,3,4,5-tetrahydro-1,4-benzoxazepine. ¹H NMR (400 MHz, DMSO-d₆): δ 12.51-12.41 (m, 1H), 8.25-8.24 (d, 1H), 7.91-7.47 (m, 5H), 7.31-6.70 (m, 3H), 4.98-4.37 (m, 2H), 4.34-3.55 (m, 4H), 3.44-3.32 (m, 2H), 2.13-1.80 (m, 3H), 1.18-1.04 (m, 3H). MS (EI) for C₂₆H₂₄FN₃O₄S: 494.2 (MH⁺).

5-(4-{[4-(Ethylsulfonyl)-3-fluoro-2-methylphenyl]carbonyl}-2,3,4,5-tetrahydro-1,4-benzoxazepin-7-yl)pyridin-2-amine. ¹H NMR (400 MHz, DMSO-d₆): δ 8.24-8.23 (d, 0.4H), 8.00-7.99 (d, 0.6H), 7.74-7.68 (m, 1.4H), 7.57-7.56 (d, 0.4H), 7.43-7.37 (m, 1.6H), 7.29-7.27 (d, 0.4H), 7.18-7.16 (d, 0.6H), 7.04-7.02 (d, 0.6H), 7.03-7.01 (d, 0.4H), 6.56-6.55 (d, 0.6H), 6.53-6.51 (d, 0.4H), 6.47-6.45 (d, 0.6H), 6.05 (bs, 2H), 4.93-4.32 (m, 2H), 4.29-3.54 (m, 4H), 3.44-3.37 (m, 2H), 2.12-1.78 (dd, 3H), 1.19-1.14 (m, 3H). MS (EI) for C₂₄H₂₄FN₃O₄S: 470.2 (MH⁺.

7-(1H-Benzimidazol-6-yl)-4-({2-ethyl-3-fluoro-4-[(2,2,2-trifluoroethyl)sulfonyl]phenyl}carbonyl)-2,3,4,5-tetrahydro-1,4-benzoxazepine. ¹H NMR (400 MHz, DMSO-d₆): δ 12.52-12.43 (m, 1H), 8.26-8.23 (m, 1H), 7.91-7.47 (m, 5H), 7.35-6.74 (m, 3H), 5.16-4.38 (m, 4H), 4.33-3.54 (m, 4H), 2.69-2.07 (m, 2H), 1.12-0.99 (m, 3H). MS (EI) for C₂₇H₂₃F₄N₃O₄S: 562.2 (MH⁺).

7-(1H-Benzimidazol-6-yl)-4-({4-[(difluoromethyl)sulfonyl]-2-ethyl-3-fluorophenyl}carbonyl)-2,3,4,5-tetrahydro-1,4-benzoxazepine. ¹H NMR (400 MHz, DMSO-d₆): δ 12.52-12.43 (m, 1H), 8.26-8.23 (m, 1H), 7.91-7.47 (m, 5H), 7.35-6.74 (m, 3H), 5.16-4.38 (m, 4H), 4.33-3.54 (m, 4H), 2.69-2.07 (m, 2H), 1.12-0.99 (m, 3H). MS (EI) for C₂₆H₂₂F₃N₃O₄S: 530.2 (MH⁺).

7-(1H-Benzimidazol-6-yl)-4-({2-ethyl-3-fluoro-4-[(fluoromethyl)sulfonyl]phenyl}carbonyl)-2,3,4,5-tetrahydro-1,4-benzoxazepine. ¹H NMR (400 MHz, DMSO-d₆): δ 12.50-12.42 (m, 1H), 8.25-8.24 (m, 1H), 7.90-7.50 (m, 5H), 7.36-6.77 (m, 3H), 5.92-5.77 (m, 2H), 5.05-4.46 (m, 2H), 4.32-3.56 (m, 4H), 2.69-2.10 (m, 2H), 1.12-0.98 (m, 3H). MS (EI) for C₂₆H₂₃F₂N₃O₄S: 512.1 (MH+).

1-[6-{[7-(1H-Benzimidazol-6-yl)-2,3-dihydro-1,4-benzoxazepin-4(5H)-yl]carbonyl}-2-fluoro-3-(methylsulfonyl)phenyl]-N,N-dimethyl-methanamine. ¹H NMR (400 MHz, DMSO-d₆): δ12.49 (s, 1H), 8.25-8.22 (m, 1H), 7.87-7.76 (m, 1H), 7.73-7.69 (m, 2H), 7.61-7.43 (m, 2H), 7.37-7.32 (m, 1H), 7.12-6.97 (m, 2H), 4.95 (m, 0.5H), 4.75 (m, 0.5H), 4.45-4.35 (m, 1H), 4.25-4.14 (m, 2H), 3.91-3.84 (m, 1H), 3.71-3.63 (m, 1H), 3.60-3.52 (m, 2H), 2.04 (s, 3H), 1.84 (s, 3H), 1.83 (s, 3H). MS (EI) for C₂₇H₂₇FN₄O₄S, found 523 (MH+).

4-{[2-Ethyl-3-fluoro-4-(methylsulfonyl)phenyl]carbonyl}-7-[4-(1H-imidazo[4,5-b]pyrazin-2-yl)phenyl]-2,3,4,5-tetrahydro-1,4-benzoxazepine. ¹H NMR (400 MHz, DMSO-d₆): δ 8.41-8.24 (m, 4H), 7.95 (d 1H), 7.87-7.66 (m, 3.5H), 7.30 (d, 0.5H), 7.16-7.11 (m, 1.5H), 6.94 (d, 0.5H), 5.06 (d, 0.5H), 4.89 (d, 0.5H), 4.52-4.02 (m, 6H), 3.65-3.54 (m, 1H), 3.40 (s, 3H), 1.13-0.98 (m, 3H). MS (EI) for C₃₀H₂₆FN₅O₄S, found 572 (MH+).

7-[4-(6-Bromo-1H-imidazo[4,5-b]pyrazin-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 8.44 (s, 1H), 8.39 (d, 1H), 8.32 (d, 1H), 7.94 (d, 1H), 7.86-7.67 (m, 4H), 7.30 (d, 0.5H), 7.16-7.11 (m, 2H), 6.93 (s, 0.5H), 5.06 (d, 0.5H), 4.89 (d, 0.5H), 4.52-4.00 (m, 5H), 3.65-3.58 (m, 1H), 3.40 (s, 3H), 2.14-2.03 (m, 1H), 1.13-0.98 (m, 3H). MS (EI) for C₃₀H₂₅BrFN₅O₄S, found 650 (MH+).

4-{[2-Ethyl-3-fluoro-4-(methylsulfonyl)phenyl]carbonyl}-7-[4-(9H-purin-8-yl)phenyl]-2,3,4,5-tetrahydro-1,4-benzoxazepine. ¹H NMR (400 MHz, DMSO-d₆): δ 9.02 (s, 1H), 8.82 (d, 1H), 8.37 (d, 1H), 8.30 (d, 1H), 7.91 (d, 1H), 7.84-7.63 (m, 4H), 7.30 (d, 0.5H), 7.16-7.10 (m, 2H), 6.91 (d, 0.5H), 5.05 (d, 0.5H), 4.88 (d, 0.5H), 4.55-4.03 (m, 5H), 3.56-3.57 (m, 1H), 3.40 (s, 3H), 2.14-2.04 (m, 1H), 1.13-0.99 (m, 3H). MS (EI) for C₃₀H₂₆FN₅O₄S, found 572 (MH+).

N-{4-[4-(4-{[2-Ethyl-3-fluoro-4-(methylsulfonyl)-phenyl]carbonyl}-2,3,4,5-tetrahydro-1,4-benzoxazepin-7-yl)phenyl]-1H-imidazol-2-yl}acetamide. ¹H NMR (400 MHz, DMSO-d₆): δ 7.99 (d, 1H), 7.92 (d, 1H), 7.80-7.56 (m, 4H), 7.50 (d, 1H), 7.29 (d, 0.5H), 7.14 (d, 1H), 7.09 (dd, 1H), 6.80 (d, 0.5H), 5.03 (d, 0.5H), 4.84 (d, 0.5H), 4.44 (d, 1H), 4.35-4.05 (m, 4H), 3.63-3.55 (m, 1H), 3.38 (s, 3H), 2.17 (s, 3H), 2.15-2.04 (m, 1H), 1.12-0.98 (m 3H). MS (EI) for C₃₀H₂₉FN₄O₅S, found 572 (MH+).

4-[4-(4-{[2-Ethyl-3-fluoro-4-(methylsulfonyl)phenyl]carbonyl}-2,3,4,5-tetrahydro-1,4-benzoxazepin-7-yl)phenyl]-1,3-thiazol-2-amine. ¹H NMR (400 MHz, DMSO-d₆): δ 7.89 (d, 0.5H), 7.81 (d, 0.5H), 7.78-7.43 (m, 6H), 7.29 (d, 0.5H), 7.15-7.05 (m, 4H), 6.79 (d, 0.5H), 5.02 (d, 0.5H), 4.84 (d, 0.5H), 4.43 (d, 1H), 4.36-4.01 (m, 4H), 3.63-3.55 (m, 1H), 3.38 (s, 3H), 2.14-2.05 (m, 1H), 1.19-0.98 (m, 3H). MS (EI) for C₂₈H₂₆FN₃O₄S₂, found 552 (MH+).

N-{4-[4-(4-{[2-Ethyl-3-fluoro-4-(methylsulfonyl)-phenyl]carbonyl}-2,3,4,5-tetrahydro-1,4-benzoxazepin-7-yl)phenyl]-1,3-thiazol-2-yl}acetamide. ¹H NMR (400 MHz, DMSO-d₆): δ 7.83-7.69 (m, 3.5H), 7.64 (d, 1H), 7.58-7.51 (1.5H), 7.40-7.38 (m, 1.5H), 7.33-7.27 (m, 1.5H), 7.14 (d, 0.5H), 7.07 (dd, 1H), 6.74 (d, 0.5H), 5.01 (d, 0.5H), 4.82 (d, 0.5H), 4.47-4.01 (m, 5H), 3.62-3.56 (m, 1H), 3.39 (s, 3H), 2.43-2.31 (m, 1H), 2.08 (s, 3H), 1.11-0.98 (m, 3H). MS (EI) for C₃₀H₂₈FN₃O₅S₂, found 594 (MH+).

4-{[2-Ethyl-3-fluoro-4-(methylsulfonyl)phenyl]carbonyl}-7-(4-fluorophenyl)-2,3,4,5-tetrahydro-1,4-benzoxazepine. ¹H NMR (400 MHz, DMSO-d₆): δ 7.78-7.67 (m, 2.5H), 7.54-7.44 (m, 2H), 7.32-7.27 (m, 1.5H), 7.23-7.19 (m, 1H), 7.15 (d, 0.5H), 7.07 (dd, 1H), 6.72 (d, 0.5H), 5.01 (d, 0.5H), 4.83 (d, 0.5H), 4.49-3.97 (m, 5H), 3.62-3.56 (m, 1H), 3.36-3.35 (m, 3H), 2.09-2.00 (m, 1H), 1.11-0.96 (m, 3H). MS (EI) for C₂₅H₂₃F₂NO₄S, found 472 (MH+).

4-{[2-Ethyl-3-fluoro-4-(methylsulfonyl)phenyl]carbonyl}-7-[4-(trifluoromethyl)phenyl]-2,3,4,5-tetrahydro-1,4-benzoxazepine. ¹H NMR (400 MHz, DMSO-d₆): δ 7.81-7.71 (m, 2H), 7.58-7.52 (m, 2H), 7.46 (d, 1H), 7.37 (d, 1H), 7.28 (d, 0.5H), 7.15 (d, 0.5H), 7.09 (d, 1H), 6.76 (d, 1H), 5.02 (d, 0.5H), 4.84 (d, 0.5H), 4.50-3.96 (m, 5H), 3.62-3.56 (m, 1H), 3.36-3.35 (m, 3H), 2.07-2.00 (m, 1H), 1.11-0.95 (m, 3H). MS (EI) for C₂₆H₂₃F₄NO₄S, found 522 (MH+).

4-{[2-Ethyl-3-fluoro-4-(methylsulfonyl)phenyl]carbonyl}-7-{4-[(trifluoromethyl)oxy]phenyl}-2,3,4,5-tetrahydro-1,4-benzoxazepine. ¹H NMR (400 MHz, DMSO-d₆): δ 7.92-7.73 (m, 5H), 7.66-7.60 (m, 2H), 7.29 (d, 0.5H), 7.15-7.11 (m, 1H), 6.84 (d, 0.5H), 5.04 (d, 0.5H), 4.86 (d, 0.5H), 4.51-4.00 (m, 5H), 3.63-3.55 (m, 1H), 3.37-3.36 (m, 3H), 2.10-2.01 (m, 1H), 1.11-0.96 (m, 3H). MS (EI) for C₂₆H₂₃F₄NO₅S, found 538 (MH+).

(7-(1H-Imidazo[4,5-b]pyridin-6-yl)-2,3-dihydrobenzo[f][1,4]oxazepin-4(5H)-yl)(4-(methylsulfonyl)phenyl)methanone. MS (EI) for C₂₃H₂₀N₄O₄S, found 449 (M+).

5-(4-{[2-Ethyl-3-fluoro-4-(methylsulfonyl)phenyl]carbonyl}-2,3,4,5-tetrahydro-1,4-benzoxazepin-7-yl)pyridin-2-amine. ¹H NMR (400 MHz, DMSO-d₆): δ 8.42-8.03 (m, 1H), 7.79-7.69 (m, 2H), 7.58-7.40 (m, 2H), 7.28-7.01 (m, 2H), 6.66-6.44 (m, 2H), 6.05 (s, 2H), 5.00-4.77 (m, 1H), 4.44-4.04 (m, 4H), 3.57-3.50 (m, 1H), 3.35 (m, 3H), 2.64 (m, 1H), 2.33-2.05 (m, 1H), 1.11-0.97 (m, 3H). MS (EI) for C₂₄H₂₄FN₃O₄S, found 470 (MH+).

1-Ethyl-3-(5-(4-(2-ethyl-3-fluoro-4-(methylsulfonyl)benzoyl)-2,3,4,5-tetrahydrobenzo[f][1,4]oxazepin-7-yl)pyridin-2-yl)urea. ¹H NMR (400 MHz, DMSO-d₆): δ 9.27 (d. 1H), 8.52-8.26 (m, 1H), 8.04 (m, 2H), 7.76 (m, 2H), 7.76 (m, 2H), 7.54-7.39 (m, 2H), 7.29-6.79 (m, 2H), 5.01-4.82 (m, 1H), 4.42-4.13 (m, 4H), 3.58 (m, 1H), 3.36 (m, 3H), 3.20 (m, 2H), 2.67 (m, 1H), 2.33-2.07 (m, 1H), 1.09-0.99 (m, 3H). MS (EI) for C₂₇H₂₉FN₄O₅S, found 541 (MH+).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-D-alaninamide. ¹H NMR (400 MHz, DMSO-d₆): δ 8.66-8.45 (m, 1H), 8.17 (m, 2H), 7.88-7.58 (m, 3H), 7.30-6.87 (m, 2H), 5.02-4.84 (m, 1H), 4.44-4.09 (m, 3H), 3.62 (m, 3H), 3.36 (m, 3H), 2.63 (m, 1H), 2.34-2.09 (m, 1H), 1.23-0.99 (m, 6H). MS (EI) for C₂₇H₂₉FN₄O₅S, found 541 (MH+).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-L-alaninamide. ¹H NMR (400 MHz, DMSO-d₆): δ 8.66-8.44 (m, 1H), 8.16 (m, 2H), 7.88-7.57 (m, 3H), 7.29-6.87 (m, 2H), 5.03-4.85 (m, 1H), 4.64-4.09 (m, 4H), 3.60 (m, 3H), 3.36 (m, 3H), 1.23-1.00 (m, 6H). MS (EI) for C₂₇H₂₉FN₄O₅S, found 541 (MH+).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-N²-methyl-L-alaninamide. ¹H NMR (400 MHz, DMSO-d₆): δ 8.66-8.44 (m, 1H), 8.15 (m, 2H), 7.90-7.59 (m, 3H), 7.29-6.88 (m, 2H), 5.03-4.84 (m, 1H), 4.44-4.09 (m, 4H), 3.60 (m, 2H), 3.36 (m, 6H), 2.28 (m, 2H), 1.23-1.00 (m, 6H). MS (EI) for C₂₈H₃₁FN₄O₅S, found 555 (MH+).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-N²-methyl-D-alaninamide. ¹H NMR (400 MHz, DMSO-d₆): δ 8.66-8.44 (m, 1H), 8.17 (m, 2H), 7.88-7.58 (m, 3H), 7.29-6.88 (m, 2H), 5.02-4.83 (m, 1H), 4.45-4.09 (m, 5H), 3.59 (m, 2H), 3.36 (m, 6H), 2.27 (m, 2H), 1.23-1.00 (m, 6H). MS (EI) for C₂₈H₃ᵢFN₄O₅S, found 555 (MH+).

N-[4-(4-{[2-Ethyl-4-(ethylsulfonyl)-3-fluorophenyl]carbonyl}-2,3,4,5-tetrahydro-1,4-benzoxazepin-7-yl)phenyl]-N²-methylglycinamide. ¹H NMR (400 MHz, DMSO-d₆): δ 9.99 (br.m, 1H), 7.66 (m, 5H), 7.53-7.29 (m, 2H), 7.13-6.68 (m, 2H), 5.00-4.64 (m, 1H), 4.41-4.09 (m, 4H), 3.71-3.41 (m, 7H), 2.36 (m, 3H), 1.19-0.98 (m, 6H). MS (EI) for C₂₅H₂₆FN₃O₅S₂, found 548 (MH+).

5-(4-{[2-Ethyl-3-fluoro-4-(methylsulfonyl)phenyl]carbonyl}-2,3,4,5-tetrahydro-1,4-benzoxazepin-7-yl)-N-(pyrrolidin-2-ylmethyl)pyridin-2-amine. ¹H NMR (400 MHz, DMSO-d₆): δ 8.28-8.08 (m, 1H), 7.78-7.41 (m, 3H), 7.28-7.03 (m, 2H), 6.65-6.51 (m, 2H), 4.98-4.78 (m, 1H), 4.45-4.02 (m, 4H), 3.57 (m, 2H), 3.39 (m, 3H), 3.20 (m, 3H), 2.76 (m, 2H), 2.32-2.03 (m, 1H), 1.77-1.37 (m, 4H), 1.09-0.97 (m, 6H). MS (EI) for C₂₉H₃₃FN₄O₄S, found 553 (MH+).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-N²-methylglycinamide. ¹H NMR (400 MHz, DMSO-d₆): δ 11.23 (m, 1H), 9.31 (br.m, 3H), 8.72-8.48 (m, 1H), 8.22-7.58 (m, 4H), 7.29-6.88 (m, 2H), 5.05-4.85 (m, 1H), 4.46-3.99 (m, 6H), 3.60 (m, 1H), 2.62 (m, 3H), 2.42-2.08 (m, 1H), 1.09-0.98 (m, 3H). MS (EI) for C₂₇H₃₁FN₄O₅S.2HCl, found 541 (MH+).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-beta-alaninamide. ¹H NMR (400 MHz, DMSO-d₆): δ 8.65-8.41 (m, 1H), 8.13 (m, 2H), 7.85-7.56 (m, 3H), 7.30-6.87 (m, 2H), 5.03-4.82 (m, 1H), 4.44-4.08 (m, 4H), 3.59 (m, 3H), 2.88-2.58 (m, 3H), 2.33-2.09 (m, 1H), 1.09-0.98 (m, 3H). MS (EI) for C₂₇H₂₉FN₄O₅S, found 541 (MH+).

N²-Ethyl-N-[5-(4-{[2-ethyl-3-fluoro-4-(methylsulfonyl)-phenyl]carbonyl}-2,3,4,5-tetrahydro-1,4-benzoxazepin-7-yl)pyridin-2-yl]glycinamide. ¹H NMR (400 MHz, DMSO-d₆): δ 8.66-8.43 (m, 1H), 8.14 (m, 2H), 7.87-7.57 (m, 3H), 7.30-6.86 (m, 2H), 5.04-4.83 (m, 1H), 4.64-4.09 (m, 5H), 3.35 (m, 3H), 2.59 (m, 3H), 2.32-2.09 (m, 1H), 1.04 (m, 6H). MS (EI) for C₂₈H₃ᵢFN₄O₅S, found 555 (MH+).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-N³-methyl-beta-alaninamide. ¹H NMR (400 MHz, DMSO-d₆): δ 10.91 (br.m. 1H), 8.65-8.43 (m, 1H), 8.11 (m, 2H), 7.84-7.58 (m, 3H), 7.28-6.87 (m, 2H), 5.04-4.84 (m, 1H), 4.44-4.08 (m, 5H), 3.58 (m, 2H), 3.36 (m, 3H), 2.79 (m, 4H), 2.32 (m, 3H), 1.09-0.99 (m, 3H). MS (EI) for C₂₈H₃₁FN₄O₅S, found 555 (MH+).

N-[5-(4-{[2-Ethyl-3-fluoro-4-(methylsulfonyl)-phenyl]carbonyl}-2,3,4,5-tetrahydro-1,4-benzoxazepin-7-yl)pyridin-2-yl]-N²,N²-dimethylglycinamide. ¹H NMR (400 MHz, DMSO-d₆): δ 10.01 (m, 1H), 8.66-8.12 (m, 3H), 7.89-7.57 (m, 3H), 7.30-7.88 (m, 3H), 5.03-4.83 (m, 1H), 4.45-4.09 (m, 4H), 3.58 (m, 1H), 3.35 (m, 3H), 3.15 (d, 2H), 2.31 (d, 6H), 2.11 (m, 1H), 1.12-0.98 (m, 3H). MS (EI) for C₂₈H₃ᵢFN₄O₅S, found 555 (MH+).

N-[5-(4-{[2-Ethyl-3-fluoro-4-(methylsulfonyl)-phenyl]carbonyl}-2,3,4,5-tetrahydro-1,4-benzoxazepin-7-yl)-1,3-thiazol-2-yl]-1-(methylamino)cyclopropanecarboxamide. ¹H NMR (400 MHz, CDCl₃): δ 10.90 (bs, 1H), 7.88-7.80 (m, 1H), 7.63-7.40 (m, 3H), 7.12-6.54 (m, 3H), 4.88 (d, 1H), 4.38-3.61 (m, 5H), 3.28 (s, 3H), 2.81-2.16 (m, 5H), 1.53-1.10 (m, 7H). MS (EI) for C₂₇H₂₉FN₄O₄S₂, found 573 (MH+).

N-(5-(4-(2-Ethyl-3-fluoro-4-(methylsulfonyl)benzoyl)-2,3,4,5-tetrahydrobenzo[f][1,4]oxazepin-7-yl)pyridin-2-yl)methanesulfonamide. ¹H NMR (400 MHz, DMSO-d₆): δ 8.58-8.36 (m, 1H), 8.09-7.54 (m, 4H), 7.29-6.81 (m, 2H), 5.02-4.83 (m, 1H), 4.46-4.06 (m, 4H), 3.57 (m, 1H), 3.36 (m, 6H), 2.66 (m, 1H), 2.27 (m, 1H), 1.10-0.98 (m, 3H). MS (EI) for C₂₅H₂₆FN₃O₅S₂, found 548 (MH+).

7-[4-(5,6-Difluoro-1H-benzimidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 13.24 (s, 1H), 8.21 (m, 2H), 7.91-7.63 (m, 6H), 7.29-6.89 (m, 3H), 5.05-4.87 (m, 1H), 4.47-4.08 (m, 4H), 3.59 (m, 1H), 3.40 (m, 3H), 2.66 (m, 1H), 2.35-2.08 (m, 1H), 1.10-1.00 (m, 3H). MS (EI) for C₃₂H₂₆F₃N₃O₄S, found 606 (MH+).

4-{[2-Ethyl-3-fluoro-4-(methylsulfonyl)phenyl]carbonyl}-7-{4-[6-(trifluoromethyl)-1H-benzimidazol-2-yl]phenyl}-2,3,4,5-tetrahydro-1,4-benzoxazepine. ¹H NMR (400 MHz, DMSO-d₆): δ 13.24 (s, 1H), 8.28 (m, 2H), 7.96-7.54 (m, 6H), 7.29-6.90 (m, 2H), 5.05-4.87 (m, 1H), 4.47-4.09 (m, 4H), 3.60 (m, 1H), 3.39 (m, 3H), 2.66 (m, 1H), 2.34-2.09 (m, 1H), 1.11-0.99 (m, 3H). MS (EI) for C₃₃H₂₇F₄N₃O₄S, found 638 (MH+).

7-[4-(6-Bromo-1H-benzimidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 13.19 (s, 1H), 8.23 (m, 2H), 7.91-7.55 (m, 6H), 7.35-6.89 (m, 3H), 5.05-4.87 (m, 1H), 4.47-4.07 (m, 4H), 3.60 (m, 1H), 3.39 (m, 3H), 2.66 (m, 1H), 2.35-2.10 (m, 1H), 1.09-0.99 (m, 3H). MS (EI) for C₃₂H₂₇BrFN₃O₄S, found 650 (MH+).

4-{[2-Ethyl-3-fluoro-4-(methylsulfonyl)phenyl]carbonyl}-7-[4-(7-methyl-1H-benzimidazol-2-yl)phenyl]-2,3,4,5-tetrahydro-1,4-benzoxazepine. ¹H NMR (400 MHz, DMSO-d₆): δ 12.88 (s, 1H), 8.21 (m, 2H), 7.88-7.63 (m, 7H), 7.17-6.90 (m, 3H), 5.04-4.87 (m, 1H), 4.47-4.07 (m, 4H), 3.60 (m, 1H), 3.40 (m, 3H), 2.65 (m, 1H), 2.44 (s, 3H), 2.33-2.07 (m, 1H), 1.10-1.00 (m, 3H). MS (EI) for C₃₃H₃₀FN₃O₄S, found 584 (MH+).

4-{[2-Ethyl-3-fluoro-4-(methylsulfonyl)phenyl]carbonyl}-7-[4-(6-methyl-1H-benzimidazol-2-yl)phenyl]-2,3,4,5-tetrahydro-1,4-benzoxazepine. ¹H NMR (400 MHz, DMSO-d₆): δ 12.83 (br.m, 1H), 8.27 (m, 2H), 7.89-7.63 (m, 6H), 7.30-6.91 (m, 4H), 5.04-4.87 (m, 1H), 4.47-4.08 (m, 4H), 3.60 (m, 1H), 3.39 (m, 3H), 2.68 (m, 1H), 2.59 (s, 3H), 2.33-2.10 (m, 1H), 1.10-1.00 (m, 3H). MS (EI) for C₃₃H₃₀FN₃O₄S, found 584 (MH+).

4-{[2-Ethyl-3-fluoro-4-(methylsulfonyl)phenyl]carbonyl}-7-[4-(3H-imidazo[4,5-b]pyridin-2-yl)phenyl]-2,3,4,5-tetrahydro-1,4-benzoxazepine. ¹H NMR (400 MHz, DMSO-d₆): δ 8.35-8.27 (m, 3H), 7.93-7.62 (m, 6H), 7.30-6.90 (m, 3H), 5.05-4.87 (m, 1H), 4.47-4.07 (m, 4H), 3.59 (m, 1H), 3.41 (s, 3H), 2.66 (m, 1H), 2.34-2.09 (m, 1H), 1.10-1.00 (m, 3H). MS (EI) for C₃₁H₂₇FN₄O₄S, found 571 (MH+).

4-{[2-Ethyl-3-fluoro-4-(methylsulfonyl)phenyl]carbonyl}-7-[4-(3H-imidazo[4,5-c]pyridin-2-yl)phenyl]-2,3,4,5-tetrahydro-1,4-benzoxazepine. ¹H NMR (400 MHz, DMSO-d₆): δ 13.41 (br.s, 1H), 8.97 (br.s, 1H), 8.33-8.25 (m, 3H), 7.82-7.63 (m, 5H), 7.30-6.92 (m, 2H), 5.05-4.87 (m, 1H), 4.47-4.06 (m, 4H), 3.59 (m, 1H), 3.40 (m, 3H), 2.66 (m, 1H), 2.34-2.09 (m, 1H), 1.10-1.00 (m, 3H). MS (EI) for C₃₁H₂₇FN₄O₄S, found 571 (MH+).

4-{[2-Ethyl-3-fluoro-4-(methylsulfonyl)phenyl]carbonyl}-7-[4-(6-fluoro-1H-benzimidazol-2-yl)phenyl]-2,3,4,5-tetrahydro-1,4-benzoxazepine. ¹H NMR (400 MHz, DMSO-d₆): δ 13.10 (s, 1H), 8.26-8.24 (d, 1H), 8.19-8.17 (d, 1H), 7.91-7.89 (d, 1H), 7.82-7.63 (m, 4H), 7.55-7.46 (m, 1H), 7.34-7.29 (m, 1H), 7.16-6.89 (m, 3H), 5.06-4.85 (m, 1H), 4.51-4.41 (m, 1H), 4.36-4.02 (m, 4H), 3.63-3.60 (m, 1H), 3.40 (s, 3H), 2.16-2.04 (m, 1H), 1.12-0.99 (m, 3H). MS (EI) for C₃₂H₂₇F₂N₃O₄S, found 588 (MH+).

7-[4-(6,7-Difluoro-1H-benzimidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 13.50 (s, 1H), 8.30-8.27 (d, 1H), 8.22-8.20 (d, 1H), 7.92-7.90 (d, 1H), 7.84-7.64 (m, 4H), 7.39-6.90 (m, 5H), 5.07-4.86 (m, 1H), 4.52-4.42 (m, 1H), 4.38-4.03 (m, 4H), 3.63-3.59 (m, 1H), 3.40 (s, 3H), 2.14-2.06 (m, 1H), 1.12-0.99 (m, 3H). MS (EI) for C₃₂H₂₆F₃N₃O₄S, found 606 (MH+).

7-[4-(5-Chloro-6-fluoro-1H-benzimidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 8.25-8.24 (d, 1H), 8.20-8.17 (d, 1H), 7.83-7.63 (m, 6H), 7.31-6.89 (m, 3H), 5.06-4.85 (m, 1H), 4.51-4.42 (m, 1H), 4.36-4.03 (m, 4H), 3.63-3.59 (m, 1H), 3.40 (s, 3H), 2.14-2.05 (s, 1H), 1.12-0.99 (m, 3H). MS (EI) for C₃₂H₂₆ClF₂N₃O₄S, found 622 (MH+).

7-[4-(6-Chloro-1H-benzimidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, d₆-DMSO): δ 8.27-8.25 (d, 1H), 8.20-8.18 (d, 1H), 7.91-7.89 (d, 1H), 7.83-7.60 (m, 6H), 7.31-6.89 (m, 4H), 5.06-4.85 (m, 1H), 4.51-4.42 (m, 1H), 4.38-4.03 (m, 4H), 3.63-3.57 (m, 1H), 3.39 (s, 3H), 2.14-2.04 (m, 1H), 1.12-0.99 (m, 3H). MS (EI) for C₃₂H₂₇ClFN₃O₄S, found 604 (MH+).

7-[4-(6,7-Dimethyl-1H-benzimidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 12.75 (m, 1H), 8.34-8.17 (m, 2H), 7.89-6.90 (m, 9H), 5.07-4.85 (m, 1H), 4.52-4.42 (m, 1H), 4.37-4.04 (m, 3H), 3.63-3.59 (m, 1H), 3.40 (s, 3H), 2.69-2.64 (m, 1H), 2.53 (s, 3H), 2.34 (s, 3H), 2.13-2.06 (m, 1H), 1.13-0.99 (m, 3H). MS (EI) for C₃₄H₃₂FN₃O₄S, found 598 (MH+).

7-[4-(5,6-Dichloro-1H-benzimidazol-2-yl)phenyl]-4-{[2-ethyl-3-fluoro-4-(methylsulfonyl)phenyl]carbonyl}-2,3,4,5-tetrahydro-1,4-benzoxazepine. ¹H NMR (400 MHz, d₆-DMSO): δ 13.32 (s, 1H), 8.28-8.19 (m, 2H), 7.97-7.90 (m, 2H), 7.83-7.64 (m, 5H), 7.30-6.9 (m, 2H), 5.06-4.86 (m, 1H) 4.51-4.04 (m, 5H), 3.4-3.36 (m, 4H), 1.12-0.99 (m, 3H). MS (EI) for C₃₂H₂₆C₁₂FN₃O₄S, found 638 (MH+).

(7-(1H-Benzo[d]imidazol-6-yl)-2,3-dihydrobenzo[f][1,4]oxazepin-4(5H)-yl)(2-ethyl-3-fluoro-4-(2-hydroxyethylsulfonyl)-phenyl)methanone. ¹H NMR (400 MHz, DMSO-d₆): δ 12.52-13.37 (m, 1H), 8.25 (m, 1H), 7.92-7.52 (m, 5H), 7.34-6.80 (m, 3H), 5.24-4.83 (m, 1H), 4.41-4.43 (m, 4H), 3.74-3.58 (m, 5H), 6.64 (m, 1H), 2.39-2.07 (m, 1H), 1.08-0.98 (m, 3H). MS (EI) for C₂₇H₂₆FN₃O₅S, found 524 (MH+).

3-[(4-{[7-(1H-Benzimidazol-6-yl)-2,3-dihydro-1,4-benzoxazepin-4(5H)-yl]carbonyl}-3-ethyl-2-fluorophenyl)sulfonyl]propan-1-ol. ¹H NMR (400 MHz, d₆-DMSO): δ 8.25-8.23 (m, 1H), 7.75-7.64 (m, 3H), 7.59-7.49 (m, 2H), 7.31-7.22 (m, 1H), 7.15-6.80 (m, 2H), 5.05 (m, 1H), 4.47-4.09 (m, 4H), 3.62-3.40 (m, 6H), 2.18-1.68 (m, 3H), 1.12-1.00 (m, 3H). MS (EI) for C₂₈H₂₈FN₃O₅S found 538 (MH+).

4-[(2-Chloro-4,5-difluorophenyl)carbonyl]-7-(2-methyl-1H-benzimidazol-5-yl)-2,3,4,5-tetrahydro-1,4-benzoxazepine. MS (EI) for C₂₄H₁₈ClF₂N₃O₂ found 454.9 (MH+).

4-[(4-Chloro-2,5-difluorophenyl)carbonyl]-7-(2-methyl-1H-benzimidazol-5-yl)-2,3,4,5-tetrahydro-1,4-benzoxazepine. MS (EI) for C₂₄H₁₈ClF₂N₃O₂ found 454.9 (MH+).

4-[(2-Bromo-3-methylphenyl)carbonyl]-7-(2-methyl-1H-benzimidazol-5-yl)-2,3,4,5-tetrahydro-1,4-benzoxazepine. MS (EI) for C₂₅H₂₂BrN₃O₂ found 477.4 (MH+).

7-(2-Methyl-1H-benzimidazol-5-yl)-4-{[4-(1H-pyrrol-1-yl)phenyl]carbonyl}-2,3,4,5-tetrahydro-1,4-benzoxazepine. MS (EI) for C₂₈H₂₄N₄O₂ found 449.5 (MH+).

4-[(1,5-Dimethyl-1H-pyrazol-3-yl)carbonyl]-7-(2-methyl-1H-benzimidazol-5-yl)-2,3,4,5-tetrahydro-1,4-benzoxazepine. MS (EI) for C₂₃H₂₃N₅O₂ found 402.5 (MH+).

7-(2-Methyl-1H-benzimidazol-5-yl)-4-{[1-(methyloxy)-naphthalen-2-yl]carbonyl}-2,3,4,5-tetrahydro-1,4-benzoxazepine. MS (EI) for C₂₉H₂₅N₃O₃ found 464.5 (MH+).

4-[(2-Bromo-5-fluorophenyl)carbonyl]-7-(2-methyl-1H-benzimidazol-5-yl)-2,3,4,5-tetrahydro-1,4-benzoxazepine. MS (EI) for C₂₄H₁₉BrFN₃O₂ found 481.3 (MH+).

7-(2-Methyl-1H-benzimidazol-5-yl)-4-{[4-(2-methylpropyl)-phenyl]carbonyl}-2,3,4,5-tetrahydro-1,4-benzoxazepine. MS (EI) for C₂₈H₂₉N₃O₂ found 440.6 (MH+).

4-(1H-Indol-6-ylcarbonyl)-7-(2-methyl-1H-benzimidazol-5-yl)-2,3,4,5-tetrahydro-1,4-benzoxazepine. MS (EI) for C₂₆H₂₂N₄O₂ found 423.5 (MH+).

4-({4-[(Difluoromethyl)oxy]phenyl}carbonyl)-7-(2-methyl-1H-benzimidazol-5-yl)-2,3,4,5-tetrahydro-1,4-benzoxazepine. MS (EI) for C₂₅H₂₁F₂N₃O₃ found 450.5 (MH+).

4-{[3-Chloro-4-(ethyloxy)phenyl]carbonyl}-7-(2-methyl-1H-benzimidazol-5-yl)-2,3,4,5-tetrahydro-1,4-benzoxazepine. MS (EI) for C₂₆H₂₄ClN₃O₃ found 462.9 (MH+).

7-(2-Methyl-1H-benzimidazol-5-yl)-4-{[2-(methyloxy)pyridin-4-yl]carbonyl}-2,3,4,5-tetrahydro-1,4-benzoxazepine. MS (EI) for C₂₄H₂₂N₄O₃ found 415.5 (MH+).

4-[(2-Fluorobiphenyl-4-yl)carbonyl]-7-(2-methyl-1H-benzimidazol-5-yl)-2,3,4,5-tetrahydro-1,4-benzoxazepine. MS (EI) for C₃₀H₂₄FN₃O₂ found 478.5 (MH+).

4-(1,3-Benzodioxol-5-ylcarbonyl)-7-(2-methyl-1H-benzimidazol-5-yl)-2,3,4,5-tetrahydro-1,4-benzoxazepine. MS (EI) for C₂₅H₂₁N₃O₄ found 428.5 (MH+).

7-(2-Methyl-1H-benzimidazol-5-yl)-4-[(4-nitrophenyl)-carbonyl]-2,3,4,5-tetrahydro-1,4-benzoxazepine. MS (EI) for C₂₄H₂₀N₄O₄ found 429.4 (MH+).

4-{[2-Fluoro-4-(trifluoromethyl)phenyl]carbonyl}-7-(2-methyl-1H-benzimidazol-5-yl)-2,3,4,5-tetrahydro-1,4-benzoxazepine. MS (EI) for C₂₅H₁₉F₄N₃O₂ found 470.4 (MH+).

7-(2-Methyl-1H-benzimidazol-5-yl)-4-[(4-propylphenyl)-carbonyl]-2,3,4,5-tetrahydro-1,4-benzoxazepine. MS (EI) for C₂₇H₂₇N₃O₂ found 426.5 (MH+).

4-[(3-Chloro-1-benzothien-2-yl)carbonyl]-7-(2-methyl-1H-benzimidazol-5-yl)-2,3,4,5-tetrahydro-1,4-benzoxazepine. MS (EI) for C₂₆H₂₀ClN₃O₂S found 475.0 (MH+).

4-{[3-Chloro-4-(methyloxy)phenyl]carbonyl}-7-(2-methyl-1H-benzimidazol-5-yl)-2,3,4,5-tetrahydro-1,4-benzoxazepine. MS (EI) for C₂₅H₂₂ClN₃O₃ found 448.9 (MH+).

7-(2-Methyl-1H-benzimidazol-5-yl)-4-{[2-methyl-4-(methyloxy)phenyl]carbonyl}-2,3,4,5-tetrahydro-1,4-benzoxazepine. MS (EI) for C₂₆H₂₅N₃O₃ found 428.5 (MH+).

4-[(4-Cyclohexylphenyl)carbonyl]-7-(2-methyl-1H-benzimidazol-5-yl)-2,3,4,5-tetrahydro-1,4-benzoxazepine. MS (EI) for C₃₀H₃₁N₃O₂ found 466.6 (MH+).

4-[(5-Chloro-2-thienyl)carbonyl]-7-(2-methyl-1H-benzimidazol-5-yl)-2,3,4,5-tetrahydro-1,4-benzoxazepine. MS (EI) for C₂₂H₁₈ClN₃O₂S found 424.9 (MH+).

4-[(5-Fluoro-2-methylphenyl)carbonyl]-7-(2-methyl-1H-benzimidazol-5-yl)-2,3,4,5-tetrahydro-1,4-benzoxazepine. MS (EI) for C₂₅H₂₂FN₃O₂ found 416.5 (MH+).

7-(2-Methyl-1H-benzimidazol-5-yl)-4-({4-[(1-methylethyl)-oxy]phenyl}carbonyl)-2,3,4,5-tetrahydro-1,4-benzoxazepine. MS (EI) for C₂₇H₂₇N₃O₃ found 442.5 (MH+).

4-[(2-Bromo-4-methylphenyl)carbonyl]-7-(2-methyl-1H-benzimidazol-5-yl)-2,3,4,5-tetrahydro-1,4-benzoxazepine. MS (EI) for C₂₅H₂₂BrN₃O₂ found 477.4 (MH+).

4-[(3-Chloro-2-methylphenyl)carbonyl]-7-(2-methyl-1H-benzimidazol-5-yl)-2,3,4,5-tetrahydro-1,4-benzoxazepine. MS (EI) for C₂₅H₂₂ClN₃O₂ found 432.9 (MH+).

6-{[7-(2-Methyl-1H-benzimidazol-5-yl)-2,3-dihydro-1,4-benzoxazepin-4(5H)-yl]carbonyl}naphthalen-2-ol. MS (EI) for C₂₈H₂₃N₃O₃ found 450.5 (MH+).

4-{[3-Fluoro-4-(methyloxy)phenyl]carbonyl}-7-(2-methyl-1H-benzimidazol-5-yl)-2,3,4,5-tetrahydro-1,4-benzoxazepine. MS (EI) for C₂₅H₂₂FN₃O₃ found 432.5 (MH+).

4-[(2,4-Dichloro-5-fluorophenyl)carbonyl]-7-(2-methyl-1H-benzimidazol-5-yl)-2,3,4,5-tetrahydro-1,4-benzoxazepine. MS (EI) for C₂₄H₁₈Cl₂FN₃O₂ found 471.3 (MH+).

4-[(2-Bromophenyl)carbonyl]-7-(2-methyl-1H-benzimidazol-5-yl)-2,3,4,5-tetrahydro-1,4-benzoxazepine. MS (EI) for C₂₄H₂₀BrN₃O₂ found 463.3 (MH+).

7-(2-Methyl-1H-benzimidazol-5-yl)-4-[(2-methylphenyl)-carbonyl]-2,3,4,5-tetrahydro-1,4-benzoxazepine. MS (EI) for C₂₅H₂₃N₃O₂ found 398.5 (MH+).

4-[(2,4-Dimethylphenyl)carbonyl]-7-(2-methyl-1H-benzimidazol-5-yl)-2,3,4,5-tetrahydro-1,4-benzoxazepine. MS (EI) for C₂₆H₂₅N₃O₂ found 412.5 (MH+).

4-[(3-Fluoro-4-methylphenyl)carbonyl]-7-(2-methyl-1H-benzimidazol-5-yl)-2,3,4,5-tetrahydro-1,4-benzoxazepine. MS (EI) for C₂₅H₂₂FN₃O₂ found 416.5 (MH+).

4-[(3,4-Dichlorophenyl)carbonyl]-7-(2-methyl-1H-benzimidazol-5-yl)-2,3,4,5-tetrahydro-1,4-benzoxazepine. MS (EI) for C₂₄H₁₉Cl₂N₃O₂ found 453.3 (MH+).

4-[(3,4-Dimethylphenyl)carbonyl]-7-(2-methyl-1H-benzimidazol-5-yl)-2,3,4,5-tetrahydro-1,4-benzoxazepine. MS (EI) for C₂₆H₂₅N₃O₂ found 412.5 (MH+).

4-{[7-(2-Methyl-1H-benzimidazol-5-yl)-2,3-dihydro-1,4-benzoxazepin-4(5H)-yl]carbonyl}benzonitrile. MS (EI) for C₂₅H₂₀N₄O₂ found 409.5 (MH+).

N,N-Dimethyl-4-{[7-(2-methyl-1H-benzimidazol-5-yl)-2,3-dihydro-1,4-benzoxazepin-4(5H)-yl]carbonyl}aniline. MS (EI) for C₂₆H₂₆N₄O₂ found 427.5 (MH+).

N,N-Diethyl-4-{[7-(2-methyl-1H-benzimidazol-5-yl)-2,3-dihydro-1,4-benzoxazepin-4(5H)-yl]carbonyl}aniline. MS (EI) for C₂₈H₃₀N₄O₂ found 455.6 (MH+).

7-(2-Methyl-1H-benzimidazol-5-yl)-4-{[4-(phenyloxy)-phenyl]carbonyl}-2,3,4,5-tetrahydro-1,4-benzoxazepine. MS (EI) for C₃₀H₂₅N₃O₃ found 476.5 (MH+).

7-(2-Methyl-1H-benzimidazol-5-yl)-4-({4-[(trifluoromethyl)-oxy]phenyl}carbonyl)-2,3,4,5-tetrahydro-1,4-benzoxazepine. MS (EI) for C₂₅H₂₀F₃N₃O₃ found 468.4 (MH+).

7-(2-Methyl-1H-benzimidazol-5-yl)-4-{[4-(methyloxy)-phenyl]carbonyl}-2,3,4,5-tetrahydro-1,4-benzoxazepine. MS (EI) for C₂₅H₂₃N₃O₃ found 414.5 (MH+).

4-{[4-(Ethylthio)phenyl]carbonyl}-7-(2-methyl-1H-benzimidazol-5-yl)-2,3,4,5-tetrahydro-1,4-benzoxazepine. MS (EI) for C₂₆H₂₅N₃O₂S found 444.6 (MH+).

Methyl 4-{[7-(2-methyl-1H-benzimidazol-5-yl)-2,3-dihydro-1,4-benzoxazepin-4(5H)-yl]carbonyl}benzoate. MS (EI) for C₂₆H₂₃N₃O₄ found 442.5 (MH+).

(4-{[7-(2-Methyl-1H-benzimidazol-5-yl)-2,3-dihydro-1,4-benzoxazepin-4(5H)-yl]carbonyl}phenyl)(phenyl)methanone. MS (EI) for C₃₁H₂₅N₃O₃ found 488.6 (MH+).

7-(2-Methyl-1H-benzimidazol-5-yl)-4-[(4-methylphenyl)-carbonyl]-2,3,4,5-tetrahydro-1,4-benzoxazepine. MS (EI) for C₂₅H₂₃N₃O₂ found 398.5 (MH+).

7-(2-Methyl-1H-benzimidazol-5-yl)-4-{[4-methyl-3-(methyloxy)phenyl]carbonyl}-2,3,4,5-tetrahydro-1,4-benzoxazepine. MS (EI) for C₂₆H₂₅N₃O₃ found 428.5 (MH+).

4-[(4-Ethylphenyl)carbonyl]-7-(2-methyl-1H-benzimidazol-5-yl)-2,3,4,5-tetrahydro-1,4-benzoxazepine. MS (EI) for C₂₆H₂₅N₃O₂ found 412.5 (MH+).

4-[(4-Butylphenyl)carbonyl]-7-(2-methyl-1H-benzimidazol-5-yl)-2,3,4,5-tetrahydro-1,4-benzoxazepine. MS (EI) for C₂₈H₂₉N₃O₂ found 440.6 (MH+).

7-(2-Methyl-1H-benzimidazol-5-yl)-4-(naphthalen-2-ylcarbonyl)-2,3,4,5-tetrahydro-1,4-benzoxazepine. MS (EI) for C₂₈H₂₃N₃O₂ found 434.5 (MH+).

7-(2-Methyl-1H-benzimidazol-5-yl)-4-[(3-methyl-2-thienyl)carbonyl]-2,3,4,5-tetrahydro-1,4-benzoxazepine. MS (EI) for C₂₃H₂₁N₃O₂S found 404.5 (MH+).

7-(2-Methyl-1H-benzimidazol-5-yl)-4-[(5-methyl-2-thienyl)carbonyl]-2,3,4,5-tetrahydro-1,4-benzoxazepine. MS (EI) for C₂₃H₂₁N₃O₂S found 404.5 (MH+).

5-{4-[(2-Bromo-4-methylphenyl)carbonyl]-2,3,4,5-tetrahydro-1,4-benzoxazepin-7-yl}pyridin-2-amine. MS (EI) for C₂₂H₂₀BrN₃O₂ found 439.3 (MH+).

5-{4-[(2-Ethylphenyl)carbonyl]-2,3,4,5-tetrahydro-1,4-benzoxazepin-7-yl}pyridin-2-amine. MS (EI) for C₂₃H₂₃N₃O₂ found 374.5 (MH+).

5-[4-(1-Benzothien-2-ylcarbonyl)-2,3,4,5-tetrahydro-1,4-benzoxazepin-7-yl]pyridin-2-amine. MS (EI) for C₂₃H₁₉N₃O₂S found 402.5 (MH+).

5-{4-[(3-Chloro-2-methylphenyl)carbonyl]-2,3,4,5-tetrahydro-1,4-benzoxazepin-7-yl}pyridin-2-amine. MS (EI) for C₂₂H₂₀ClN₃O₂ found 394.9 (MH+).

5-{4-[(3-Fluoro-2-methylphenyl)carbonyl]-2,3,4,5-tetrahydro-1,4-benzoxazepin-7-yl}pyridin-2-amine. MS (EI) for C₂₂H₂₀FN₃O₂ found 378.4 (MH+).

5-{4-[(2,4-Dichloro-5-fluorophenyl)carbonyl]-2,3,4,5-tetrahydro-1,4-benzoxazepin-7-yl}pyridin-2-amine. MS (EI) for C₂₁H₁₆Cl₂FN₃O₂ found 433.3 (MH+).

5-(4-{[1-(Methyloxy)naphthalen-2-yl]carbonyl}-2,3,4,5-tetrahydro-1,4-benzoxazepin-7-yl)pyridin-2-amine. MS (EI) for C₂₆H₂₃N₃O₃ found 426.5 (MH+).

5-{4-[(4-Bromo-2-fluorophenyl)carbonyl]-2,3,4,5-tetrahydro-1,4-benzoxazepin-7-yl}pyridin-2-amine. MS (EI) for C₂₁H₁₇BrFN₃O₂ found 443.3 (MH+).

5-(4-{[4-(2-Methylpropyl)phenyl]carbonyl}-2,3,4,5-tetrahydro-1,4-benzoxazepin-7-yl)pyridin-2-amine. MS (EI) for C₂₅H₂₇N₃O₂ found 402.5 (MH+).

5-[4-(1H-Indol-6-ylcarbonyl)-2,3,4,5-tetrahydro-1,4-benzoxazepin-7-yl]pyridin-2-amine. MS (EI) for C₂₃H₂₀N₄O₂ found 385.4 (MH+).

5-[4-({4-[(Difluoromethyl)oxy]phenyl}carbonyl)-2,3,4,5-tetrahydro-1,4-benzoxazepin-7-yl]pyridin-2-amine. MS (EI) for C₂₂H₁₉F₂N₃O₃ found 412.4 (MH+).

5-(4-{[3-Fluoro-4-(trifluoromethyl)phenyl]carbonyl}-2,3,4,5-tetrahydro-1,4-benzoxazepin-7-yl)pyridin-2-amine. MS (EI) for C₂₂H₁₇F₄N₃O₂ found 432.4 (MH+).

5-{4-[(3-Bromo-2-methylphenyl)carbonyl]-2,3,4,5-tetrahydro-1,4-benzoxazepin-7-yl}pyridin-2-amine. MS (EI) for C₂₂H₂₀BrN₃O₂ found 439.3 (MH+).

5-[4-(2-Thienylcarbonyl)-2,3,4,5-tetrahydro-1,4-benzoxazepin-7-yl]pyridin-2-amine. MS (EI) for C₁₉H₁₇N₃O₂S found 352.4 (MH+).

5-{4-[(3-Methyl-2-thienyl)carbonyl]-2,3,4,5-tetrahydro-1,4-benzoxazepin-7-yl}pyridin-2-amine. MS (EI) for C₂₀H₁₉N₃O₂S found 366.5 (MH+).

5-{4-[(5-Methyl-2-thienyl)carbonyl]-2,3,4,5-tetrahydro-1,4-benzoxazepin-7-yl}pyridin-2-amine. MS (EI) for C₂₀H₁₉N₃O₂S found 366.5 (MH+).

5-[4-(1H-Indol-5-ylcarbonyl)-2,3,4,5-tetrahydro-1,4-benzoxazepin-7-yl]pyridin-2-amine. MS (EI) for C₂₃H₂₀N₄O₂ found 385.4 (MH+).

5-[4-(1,3-Benzodioxol-5-ylcarbonyl)-2,3,4,5-tetrahydro-1,4-benzoxazepin-7-yl]pyridin-2-amine. MS (EI) for C₂₂H₁₉N₃O₄ found 390.4 (MH+).

5-{4-[(4-Propylphenyl)carbonyl]-2,3,4,5-tetrahydro-1,4-benzoxazepin-7-yl}pyridin-2-amine. MS (EI) for C₂₄H₂₅N₃O₂ found 388.5 (MH+).

5-{4-[(3-Chloro-1-benzothien-2-yl)carbonyl]-2,3,4,5-tetrahydro-1,4-benzoxazepin-7-yl}pyridin-2-amine. MS (EI) for C₂₃H₁₈ClN₃O₂S found 436.9 (MH+).

5-(4-{[2-Methyl-4-(methyloxy)phenyl]carbonyl}-2,3,4,5-tetrahydro-1,4-benzoxazepin-7-yl)pyridin-2-amine. MS (EI) for C₂₃H₂₃N₃O₃ found 390.5 (MH+).

5-{4-[(4-Cyclohexylphenyl)carbonyl]-2,3,4,5-tetrahydro-1,4-benzoxazepin-7-yl}pyridin-2-amine. MS (EI) for C₂₇H₂₉N₃O₂ found 428.5 (MH+).

5-{4-[(4-Bromo-3-methylphenyl)carbonyl]-2,3,4,5-tetrahydro-1,4-benzoxazepin-7-yl}pyridin-2-amine. MS (EI) for C₂₂H₂₀BrN₃O₂ found 439.3 (MH+).

5-{4-[(4-Bromo-2-methylphenyl)carbonyl]-2,3,4,5-tetrahydro-1,4-benzoxazepin-7-yl}pyridin-2-amine. MS (EI) for C₂₂H₂₀BrN₃O₂ found 439.3 (MH+).

5-{4-[(5-Fluoro-2-methylphenyl)carbonyl]-2,3,4,5-tetrahydro-1,4-benzoxazepin-7-yl}pyridin-2-amine. MS (EI) for C₂₂H₂₀FN₃O₂ found 378.4 (MH+).

5-[4-(Phenylcarbonyl)-2,3,4,5-tetrahydro-1,4-benzoxazepin-7-yl]pyridin-2-amine. MS (EI) for C₂₁H₁₉N₃O₂ found 346.4 (MH+).

5-{4-[(2-Chlorophenyl)carbonyl]-2,3,4,5-tetrahydro-1,4-benzoxazepin-7-yl}pyridin-2-amine. MS (EI) for C₂₁H₁₈ClN₃O₂ found 380.8 (MH+).

5-{4-[(2,3-Dimethylphenyl)carbonyl]-2,3,4,5-tetrahydro-1,4-benzoxazepin-7-yl}pyridin-2-amine. MS (EI) for C₂₃H₂₃N₃O₂ found 374.5 (MH+).

5-{4-[(2,4-Dimethylphenyl)carbonyl]-2,3,4,5-tetrahydro-1,4-benzoxazepin-7-yl}pyridin-2-amine. MS (EI) for C₂₃H₂₃N₃O₂ found 374.5 (MH+).

5-{4-[(3-Fluoro-4-methylphenyl)carbonyl]-2,3,4,5-tetrahydro-1,4-benzoxazepin-7-yl}pyridin-2-amine. MS (EI) for C₂₂H₂₀FN₃O₂ found 378.4 (MH+).

5-{4-[(3,4-Dichlorophenyl)carbonyl]-2,3,4,5-tetrahydro-1,4-benzoxazepin-7-yl}pyridin-2-amine. MS (EI) for C₂₁H₁₇Cl₂N₃O₂ found 415.3 (MH+).

5-{4-[(3-M ethylphenyl)carbonyl]-2,3,4,5-tetrahydro-1,4-benzoxazepin-7-yl}pyridin-2-amine. MS (EI) for C₂₂H₂₁N₃O₂ found 360.4 (MH+).

5-{4-[(3,4-Dimethylphenyl)carbonyl]-2,3,4,5-tetrahydro-1,4-benzoxazepin-7-yl}pyridin-2-amine. MS (EI) for C₂₃H₂₃N₃O₂ found 374.5 (MH+).

5-[4-({4-[(Trifluoromethyl)oxy]phenyl}carbonyl)-2,3,4,5-tetrahydro-1,4-benzoxazepin-7-yl]pyridin-2-amine. MS (EI) for C₂₂H₁₈F₃N₃O₃ found 430.4 (MH+).

5-(4-{[4-(Methyloxy)phenyl]carbonyl}-2,3,4,5-tetrahydro-1,4-benzoxazepin-7-yl)pyridin-2-amine. MS (EI) for C₂₂H₂₁N₃O₃ found 376.4 (MH+).

5-(4-{[4-(Ethyloxy)phenyl]carbonyl}-2,3,4,5-tetrahydro-1,4-benzoxazepin-7-yl)pyridin-2-amine. MS (EI) for C₂₃H₂₃N₃O₃ found 390.5 (MH+).

5-(4-{[4-(Methylthio)phenyl]carbonyl}-2,3,4,5-tetrahydro-1,4-benzoxazepin-7-yl)pyridin-2-amine. MS (EI) for C₂₂H₂₁N₃O₂S found 392.5 (MH+).

5-(4-{[4-(Ethylthio)phenyl]carbonyl}-2,3,4,5-tetrahydro-1,4-benzoxazepin-7-yl)pyridin-2-amine. MS (EI) for C₂₃H₂₃N₃O₂S found 406.5 (MH+).

Methyl 4-{[7-(6-aminopyridin-3-yl)-2,3-dihydro-1,4-benzoxazepin-4(5H)-yl]carbonyl}benzoate. MS (EI) for C₂₃H₂ᵢN₃O₄ found 404.4 (MH+).

5-(4-{[4-(Trifluoromethyl)phenyl]carbonyl}-2,3,4,5-tetrahydro-1,4-benzoxazepin-7-yl)pyridin-2-amine. MS (EI) for C₂₂H₁₈F₃N₃O₂ found 414.4 (MH+).

5-(4-{[4-(1,1-Dimethylethyl)phenyl]carbonyl}-2,3,4,5-tetrahydro-1,4-benzoxazepin-7-yl)pyridin-2-amine. MS (EI) for C₂₅H₂₇N₃O₂ found 402.5 (MH+).

5-{4-[(4-Methylphenyl)carbonyl]-2,3,4,5-tetrahydro-1,4-benzoxazepin-7-yl}pyridin-2-amine. MS (EI) for C₂₂H₂ᵢN₃O₂ found 360.4 (MH+).

5-{4-[(4-Ethylphenyl)carbonyl]-2,3,4,5-tetrahydro-1,4-benzoxazepin-7-yl}pyridin-2-amine. MS (EI) for C₂₃H₂₃N₃O₂ found 374.5 (MH+).

5-{4-[(4-Pentylphenyl)carbonyl]-2,3,4,5-tetrahydro-1,4-benzoxazepin-7-yl}pyridin-2-amine. MS (EI) for C₂₆H₂₉N₃O₂ found 416.5 (MH+).

5-{4-[(1-Methyl-1H-pyrrol-2-yl)carbonyl]-2,3,4,5-tetrahydro-1,4-benzoxazepin-7-yl}pyridin-2-amine. MS (EI) for C₂₀H₂₀N₄O₂ found 349.4 (MH+).

5-[4-(Naphthalen-2-ylcarbonyl)-2,3,4,5-tetrahydro-1,4-benzoxazepin-7-yl]pyridin-2-amine. MS (EI) for C₂₅H₂ᵢN₃O₂ found 396.5 (MH+).

7-(1H-Benzimidazol-6-yl)-4-[(4-bromo-2-methylphenyl)-carbonyl]-2,3,4,5-tetrahydro-1,4-benzoxazepine. ¹H NMR (400 MHz, DMSO-d₆): δ 12.48 (br s, 1H), 8.21 (s, 1H), 7.89-7.37 (m, 6H), 7.27-6.60 (m, 3H), 4.97-4.75 (m, 1H), 4.40 (s, 1H), 4.29-3.93 (m, 3H), 3.53 (br s, 1H), 0.96-0.89 (m, 3H); MS (EI) for C₂₄H₂₀BrN₃O₂: 462, 464 (MH+).

7-(1H-Benzimidazol-6-yl)-4-[(2-bromo-4-methylphenyl)-carbonyl]-2,3,4,5-tetrahydro-1,4-benzoxazepine. ¹H NMR (400 MHz, DMSO-d₆): δ 12.52 (br s, 1H), 8.25 (s, 1H), 7.97-7.45 (m, 5H), 7.36-6.59 (m, 4H), 5.03-4.68 (m, 1H), 4.55-3.91 (m, 4H), 3.64-3.44 (m, 1H), 1.01-0.87 (m, 3H); MS (EI) for C₂₄H₂₀BrN₃O₂: 462-464 (MH+).

7-(1H-Benzimidazol-6-yl)-4-[(2-methylphenyl)carbonyl]-2,3,4,5-tetrahydro-1,4-benzoxazepine. ¹H NMR (400 MHz, DMSO-d₆): δ 12.53 (br s, 1H), 8.25 (s, 1H), 7.95-6.50 (m, 10H), 5.00-4.77 (m, 1H), 4.44-3.91 (m, 4H), 3.56 (br s, 1H), 2.16-1.85 (m, 3H); MS (EI) for C₂₄H₂₁N₃O₂: 384 (MH⁺).

7-(1H-Benzimidazol-6-yl)-4-[(4-ethynylphenyl)carbonyl]-2,3,4,5-tetrahydro-1,4-benzoxazepine. ¹H NMR (400 MHz, DMSO-d₆): δ 12.53 (br s, 1H), 8.25 (s, 1H), 7.97-7.22 (m, 9H), 7.14-6.93 (m, 1H), 4.87 (s, 1H), 4.63-3.87 (m, 5H), 3.73 (br s, 1H); MS (EI) for C₂₄H₁₉N₃O₂: 394 (MH⁺).

7-(1H-Benzimidazol-6-yl)-4-[(4-methylphenyl)carbonyl]-2,3,4,5-tetrahydro-1,4-benzoxazepine. ¹H NMR (400 MHz, DMSO-d₆): δ 12.53 (br s, 1H), 8.25 (s, 1H), 7.94-6.83 (m, 10H), 4.91-4.49 (m, 2H), 4.33-4.06 (m, 2H), 4.06-3.69 (m, 2H), 2.42-2.25 (m, 3H); MS (EI) for C₂₄H₂ᵢN₃O₂: 384 (MH⁺).

7-(1H-Benzimidazol-6-yl)-4-{[2-methyl-4-(methylsulfonyl)-phenyl]carbonyl}-2,3,4,5-tetrahydro-1,4-benzoxazepine. ¹H NMR (400 MHz, CDCl₃): δ 8.16-7.77 (m, 4H), 7.58-7.10 (m, 6H), 6.30 (s, 1H), 4.43-4.26 (m, 4H), 4.21-4.02 (m, 2H), 3.21-3.18 (m, 3H), 2.15-1.95 (m, 3H); MS (EI) for C₂₅H₂₃N₃O₄S: 460 (M−H).

7-(1H-Benzimidazol-6-yl)-4-{[2-bromo-4-(methylsulfonyl)-phenyl]carbonyl}-2,3,4,5-tetrahydro-1,4-benzoxazepine. ¹H NMR (400 MHz, CDCl₃): δ 8.35 (s 1H), 8.14-8.05 (m, 2H), 7.84-7.77 (m, 1H), 7.59-7.31 (m, 4H), 7.25-7.13 (m, 2H), 6.35 (s, 1H), 4.48-4.21 (m, 4H), 4.20-4.04 (m, 2H), 3.23 (s, 3H); MS (EI) for C₂₄H₂₀BrN₃O₄S: 526 (MH⁺).

7-(1H-Benzimidazol-6-yl)-4-[(4-iodophenyl)carbonyl]-2,3,4,5-tetrahydro-1,4-benzoxazepine. ¹H NMR (400 MHz, CDCl₃): δ 8.15-8.06 (s 1H), 7.84-7.35 (m, 7H), 7.28 (s, 1H), 7.19-6.82 (m, 3H), 4.93-4.84 (m, 1H), 4.57-4.48 (m, 1H), 4.33-4.03 (m, 3H), 3.85 (br s, 1H); MS (EI) for C₂₃H₁₈1N₃O₂: 496 (MH⁺).

7-(1H-Benzimidazol-6-yl)-4-[(4-chloro-2-methylphenyl)-carbonyl]-2,3,4,5-tetrahydro-1,4-benzoxazepine hydrochloride salt. ¹H NMR (400 MHz, CDCl₃): δ 8.10 (s 1H), 7.82-7.31 (m, 6H), 7.25-7.04 (m, 3H), 6.63 (s, 1H), 5.10-4.72 (m, ˜1H), 4.49-3.94 (m, ˜4H), 3.73-3.61 (m, ˜1H), 2.02 (s, 3H); MS (EI) for C₂₄H₂₀ClN₃O₂: 418 (MH⁺).

7-(1H-Benzimidazol-6-yl)-4-{[4-(1-methylethyl)phenyl]-carbonyl}-2,3,4,5-tetrahydro-1,4-benzoxazepine hydrochloride salt. ¹H NMR (400 MHz, CDCl₃): δ 8.08 (s, 1H), 7.79-7.63 (m, 3H), 7.56-7.44 (m, 2H), 7.41-7.22 (m, ˜4H), 7.14 (s, 1H), 6.86 (s, ˜1H), 4.90 (s, 1H), 4.54 (s, 1H), 4.32-3.99 (m, 3H), 3.91 (s, 1H), 2.95 (br s, 1H), 1.26 (s, 6H); MS (EI) for C₂₆H₂₅N₃O₂: 418 (MH⁺).

7-(1H-Benzimidazol-6-yl)-4-[(2,3-dimethylphenyl)carbonyl]-2,3,4,5-tetrahydro-1,4-benzoxazepine. ¹H NMR (400 MHz, CDCl₃): δ 8.06-7.91 (m, 1H), 7.71-7.60 (m, 2H), 7.55-7.43 (m, 2H), 7.30-6.56 (m, 6H), 5.09-4.87 (m, 1H), 4.47-4.16 (m, 3H), 4.13 (m, 1H), 3.75-3.61 (m, 1H), 2.27 (s, 1H), 2.17-2.13 (m, 2.5H), 2.12 (s, 1H), 1.88 (s, 1.5H); MS (EI) for C₂₅H₂₃N₃O₂: 398 (MH⁺).

7-(1H-Benzimidazol-6-yl)-4-[(3-fluoro-2-methylphenyl)-carbonyl]-2,3,4,5-tetrahydro-1,4-benzoxazepine. ¹H NMR (400 MHz, CDCl₃): δ 8.20-7.27 (m, 6H), 7.22-6.60 (m, 5H), 5.12-4.71 (m, 1H), 4.52-3.93 (m, 4H), 3.65 (s, 1H), 2.17 (s, 1H), 1.97 (s, 2H); MS (EI) for C₂₄H₂₀FN₃O₂: 402 (MH⁺).

2-[(4-{[7-(1H-Benzimidazol-6-yl)-2,3-dihydro-1,4-benzoxazepin-4(5H)-yl]carbonyl}phenyl)oxy]ethanol. ¹H NMR (400 MHz, CDCl₃): δ 8.09-8.00 (m, 1H), 7.71-7.27 (m, 7H), 7.19-6.60 (m, 4H), 4.96-3.85 (m, 10H), 3.05 (br s, 1H); MS (EI) for C₂₅H₂₃N₃O₄: 430 (MH⁺).

1,1-Dimethylethyl {2-[(4-{[7-(1H-benzimidazol-6-yl)-2,3-dihydro-1,4-benzoxazepin-4(5H)-yl]carbonyl}phenyl)oxy]ethyl}carbamate. ¹H NMR (400 MHz, CDCl₃): δ 8.11 (s, 1H), 7.89-7.40 (m, 4H), 7.37-7.30 (m, 2H), 7.16-7.08 (m, 1H), 7.02-6.81 (m, 3H), 5.20-4.56 (m, 2H), 4.30-3.83 (m, 6H), 3.57 (s, 2H), 1.469 (s, 9H); MS (EI) for C₃₀H₃₂N₄O₅: 529 (MH⁺).

2-(4-{[7-(1H-Benzimidazol-6-yl)-2,3-dihydro-1,4-benzoxazepin-4(5H)-yl]carbonyl}phenyl)ethanol. ¹H NMR (400 MHz, CDCl₃): δ 8.02-7.92 (m, 1H), 7.70-7.22 (m, 9H), 7.15-7.08 (m, 1H), 6.68 (s, 1H), 4.90 (s, 1H), 4.50 (s, 2H), 4.28-4.04 (m, 6H), 3.98-3.83 (m, 2H); MS (EI) for C₂₅H₂₃N₃O₃: 414 (MH⁺).

7-(1H-Benzimidazol-6-yl)-4-{[4-(trifluoromethyl)-phenyl]carbonyl}-2,3,4,5-tetrahydro-1,4-benzoxazepine hydrochloride salt. ¹H NMR (400 MHz, DMSO-d₆): δ 9.54-9.43 (m, 1H), 8.11-7.75 (m, 6H), 7.70-7.46 (m, 3H), 7.20-6.90 (m, 1H), 4.96-4.54 (m, 2H), 4.38-4.15 (m, 2H), 4.10-3.70 (m, 2H); MS (EI) for C₂₄H₁₈F₃N₃O₃: 438 (MH⁺).

7-(1H-Benzimidazol-6-yl)-4-(pyridin-4-ylcarbonyl)-2,3,4,5-tetrahydro-1,4-benzoxazepine. ¹H NMR (400 MHz, DMSO-d₆): δ 8.75-8.45 (m, 2H), 7.90-6.90 (m, 10H), 4.93-4.49 (m, 2H), 4.45-4.20 (m, 2H), 4.19-3.99 (m, 2H); MS (EI) for C₂₂H₁₈N₄O₂: 371 (MH⁺).

7-(1H-Benzimidazol-6-yl)-4-(pyridin-3-ylcarbonyl)-2,3,4,5-tetrahydro-1,4-benzoxazepine. ¹H NMR (400 MHz, DMSO-d₆): δ 8.78-8.39 (m, 3H), 7.90-7.36 (m, 7H), 7.24-6.92 (m, 2H), 4.94-4.54 (m, 2H), 4.38-4.14 (m, 2H), 4.08-3.74 (m, 2H); MS (EI) for C₂₂H₁₈N₄O₂: 371 (MH⁺).

7-(1H-Benzimidazol-6-yl)-4-[(4-fluoro-2-methylphenyl)carbonyl]-2,3,4,5-tetrahydro-1,4-benzoxazepine. ¹H NMR (400 MHz, DMSO-d₆): δ 8.25 (d, 1H), 7.94-7.46 (m, 4H), 7.33-7.03 (m, 4H), 6.69 (d, 1H), 4.89 (br d, 1H), 4.42 (br s, 1H), 4.30-3.98 (m, 3H), 3.56 (br s, 1H), 1.91 (s, 3H). MS (EI) for C₂₄H₂₀FN₃O₂: 402 (M+H).

4-{[7-(1H-Benzimidazol-6-yl)-2,3-dihydro-1,4-benzoxazepin-4(5H)-yl]carbonyl}-N-cyclopentylbenzenesulfonamide. ¹H NMR (400 MHz, DMSO-d₆): δ 8.30 (d, 1H), 7.89-7.74 (m, 3H), 7.70-7.42 (m, 5H), 7.25 (d, 1H), 7.10-7.03 (m, 1H), 6.74 (s, 1H), 4.86 (s, 1H), 4.50 (s, 1H), 4.29-3.99 (m, 3H), 3.70 (br s, 1H), 3.38 (m, 1H), 1.63-1.10 (m, 8H). MS (EI) for C₂₈H₂₈N₄O₄S: 517 (M+H).

7-(1H-Benzimidazol-6-yl)-4-[(1,1-dioxido-2,3-dihydro-1-benzothien-5-yl)carbonyl]-2,3,4,5-tetrahydro-1,4-benzoxazepine. ¹H NMR (400 MHz, DMSO-d₆): δ 8.54 (d, 1H), 7.88-7.65 (m, 3H), 7.61-7.49 (m, 3H), 7.44-7.32 (m, 1.5H), 7.10 (t, 1H), 6.99 (s, 0.5H), 4.88 (s, 1.2H), 4.55 (s, 0.8H), 4.30 (br s, 0.8H), 4.16 (br s, 1.2H), 4.04 (br s, 0.8H), 3.73 (br s, 1.2H), 3.65-3.55 (m, 2H), 3.27-3.21 (m, 2H). MS (EI) for C₂₅H₂ᵢN₃O₄S: 460 (M+H).

4-{[7-(1H-Benzimidazol-6-yl)-2,3-dihydro-1,4-benzoxazepin-4(5H)-yl]carbonyl}-N-[2-(diethylamino)ethyl]benzenesulfonamide. ¹H NMR (400 MHz, DMSO-d₆): δ 10.08 (br s, 1H), 9.56-9.47 (m, 1H), 8.37-8.28 (m, 1H), 8.07-7.48 (m, 8.5H), 7.17-7.10 (m, 1H), 6.96 (s, 0.5H), 4.92 (s, 1.3H), 4.58 (s, 0.7H), 4.37-4.01 (m, 3H), 3.75 (br s, 1H), 3.19-3.06 (m, 8H), 1.21-1.12 (m, 6H). MS (EI) for C₂₉H₃₃N₅O₄S: 548 (M+H).

4-{[7-(1H-Benzimidazol-6-yl)-2,3-dihydro-1,4-benzoxazepin-4(5H)-yl]carbonyl}-N-(2-morpholin-4-ylethyl)benzenesulfonamide. ¹H NMR (400 MHz, DMSO-d₆): δ 11.06 (br s, 1H), 9.63 (s, 1H), 8.40-8.27 (m, 1H), 8.05 (s, 0.5H), 7.97-7.87 (m, 4H), 7.78 (s, 0.5H), 7.67-7.59 (m, 3H), 7.50 (d, 0.5H), 7.18-7.10 (m, 1H), 6.97 (s, 0.5H), 4.92 (s, 1.3H), 4.59 (s, 0.7H), 4.36-4.18 (m, 2H), 4.06-3.30 (m, 14H). MS (EI) for C₂₉H₃ᵢN₅O₅S: 562 (M+H).

N-(4-{[7-(1H-Benzimidazol-6-yl)-2,3-dihydro-1,4-benzoxazepin-4(5H)-yl]carbonyl}phenyl)methanesulfonamide. ¹H NMR (400 MHz, DMSO-d₆): δ 10.11 (s, 1H), 9.62 (s, 1H), 8.08-7.58 (m, 5H), 7.44-6.97 (m, 6H), 4.93-3.67 (m, 6H), 3.04 (br s, 3H). MS (EI) for C₂₄H₂₂N₄O₄S: 463 (M+H).

7-(1H-Benzimidazol-6-yl)-4-{[3-methyl-4-(methylsulfonyl)-phenyl]carbonyl}-2,3,4,5-tetrahydro-1,4-benzoxazepine. ¹H NMR (400 MHz, DMSO-d₆): δ 9.54 (d, 1H), 8.03 (s, 0.5H), 7.96-7.84 (m, 3H), 7.78 (d, 1H), 7.66-7.60 (m, 1.5H), 7.51-7.44 (m, 1H), 7.37 (d, 0.5H), 7.31 (s, 0.5H), 7.20-7.11 (m, 2H), 7.07-7.00 (m, 0.5H), 4.90 (s, 1.2H), 4.56 (s, 0.8H), 4.34 (br s, 0.8H), 4.21-4.16 (m, 1.2H), 4.04 (br s, 0.8H), 3.77-3.71 (m, 1.2H), 3.25 (s, 1.8H), 3.21 (s, 1.2H), 2.65 (s, 1.8H), 2.46 (s, 1.2H). MS (EI) for C₂₅H₂₃N₃O₄S: 462 (M+H).

7-(1H-Benzimidazol-6-yl)-4-[(4-fluoro-3-methylphenyl)-carbonyl]-2,3,4,5-tetrahydro-1,4-benzoxazepine. ¹H NMR (400 MHz, DMSO-d₆): δ 9.62 (s, 1H), 8.07-7.58 (m, 4H), 7.65-7.59 (m, 1H), 7.40-7.05 (m, 5H), 4.87 (br s, 1.2H), 4.59 (br s, 0.8H), 4.19 (br s, 1.2H), 4.00 (br s, 0.8H), 3.79 (br s, 1.2H), 2.25 (s, 2.5H), 2.10 (br s, 2.5H). MS (EI) for C₂₄H₂₀FN₃O₂: 402 (M+H).

7-(1H-Benzimidazol-6-yl)-4-[(2,4-dichlorophenyl)carbonyl]-2,3,4,5-tetrahydro-1,4-benzoxazepine. ¹H NMR (400 MHz, CD₃CN): δ 8.54-8.41 (m, 2H), 8.04-6.80 (m, 8H), 4.98-4.82 (m, 1H), 4.55-4.00 (m, 4H), 3.72-3.58 (m, 1H); MS (EI) for C₂₃H₁₇Cl₂N₃O₂: 439.1 (MH+).

7-(1H-Benzimidazol-6-yl)-4-{[4-(pyrrolidin-1-ylsulfonyl)-phenyl]carbonyl}-2,3,4,5-tetrahydro-1,4-benzoxazepine. ¹H NMR (400 MHz, DMSO-d₆): δ 8.42-8.29 (m, 1H), 7.91-7.82 (m, 3H), 7.75-6.75 (m, 7H), 4.89 (s, 1H), 4.52 (s, 1H), 4.29 (s, 1H), 4.17 (s, 1H), 4.05 (s, 1H), 3.73 (s, 1H), 3.21-3.06 (m, 4H), 1.73-1.43 (m, 4H); MS (EI) for C₂₇H₂₆N₄O₄S: 503.2 (MH+).

7-(1H-Benzimidazol-6-yl)-4-{[4-(morpholin-4-ylsulfonyl)-phenyl]carbonyl}-2,3,4,5-tetrahydro-1,4-benzoxazepine. ¹H NMR (400 MHz, CD₃CN): δ 8.32-8.20 (m, 1H), 7.95-7.37 (m, 9H), 7.18-6.81 (m, 1H), 4.90 (s, 1H), 4.49 (s, 1H), 4.30 (m, 1H), 4.16-4.08 (m, 2H), 3.81-3.74 (m, 1H), 3.73-3.60 (m, 4H), 3.02-2.93 (m, 4H); MS (EI) for C₂₇H₂₆N₄O₅S: 519.2 (MH+).

7-(1H-Benzimidazol-6-yl)-4-(furan-2-ylcarbonyl)-2,3,4,5-tetrahydro-1,4-benzoxazepine. ¹H NMR (400 MHz, DMSO-d₆): δ 8.28 (s, 1H), 7.98-7.36 (m, 6H), 7.1-6.98 (m, 2H), 6.65 (s, 1H), 5.10-4.76 (m, 2H), 4.33-3.91 (m, 4H); MS (EI) for C₂ᵢH₁₇N₃O₃: 360.2 (MH+).

7-(1H-Benzimidazol-6-yl)-4-[(1-methyl-1H-indol-6-yl)carbonyl]-2,3,4,5-tetrahydro-1,4-benzoxazepine. ¹H NMR (400 MHz, DMSO-d₆): δ 12.50 (s, 1H), 8.24 (s, 1H), 7.93-6.94 (m, 10H), 6.47 (m, 1H), 4.95-4.55 (m, 2H), 4.42-3.69 (m, 6H), 3.53-3.37 (m, 1H); MS (EI) for C₂₆H₂₂N₄O₂: 423.2 (MH+).

7-(1H-Benzimidazol-6-yl)-4-(1-benzofuran-3-ylcarbonyl)-2,3,4,5-tetrahydro-1,4-benzoxazepine. ¹H NMR (400 MHz, DMSO-d₆): δ 12.56-12.45 (m, 1H), 8.45-8.15 (m, H), 7.94-7.24 (m, 9H), 7.10-7.03 (m, 1H), 4.98-4.62 (m, 2H), 4.37-3.90 (m, 4H); MS (EI) for C₂₅H₁₉N₃O₃: 410.2 (MH+).

7-(1H-Benzimidazol-6-yl)-4-(1-benzofuran-2-ylcarbonyl)-2,3,4,5-tetrahydro-1,4-benzoxazepine. ¹H NMR (400 MHz, DMSO-d₆): δ 12.57-12.46 (m, 1H), 8.25 (s, 1H), 7.95-7.23 (m, 10H), 7.11-7.03 (m, 1H), 5.12-4.80 (m, 2H), 4.35-3.95 (m, 4H); MS (EI) for C₂₅H₁₉N₃O₃: 410.2 (MH+).

7-(1H-Benzimidazol-6-yl)-4-(2-thienylcarbonyl)-2,3,4,5-tetrahydro-1,4-benzoxazepine. ¹H NMR (400 MHz, DMSO-d₆): δ 12.54 (s, 1H), 8.25 (s, 1H), 7.92-7.37 (m, 7H), 7.19-7.02 (m, 2H), 4.89 (s, 2H), 4.34-4.25 (m, 2H), 4.07 (s, 2H); MS (EI) for C₂₁H₁₇N₃O₂S: 376.1 (MH+).

7-(1H-Benzimidazol-6-yl)-4-(3-thienylcarbonyl)-2,3,4,5-tetrahydro-1,4-benzoxazepine. ¹H NMR (400 MHz, DMSO-d₆): δ 8.24 (s, 1H), 7.90-7.57 (m, 5H), 7.56-7.31 (m, 2H), 7.26-6.93 (m, 2H), 4.92-4.62 (m, 2H), 4.32-3.81 (m, 4H); MS (EI) for C₂₁H₁₇N₃O₂S: 376.1 (MH+).

7-(1H-Benzimidazol-6-yl)-4-(furan-3-ylcarbonyl)-2,3,4,5-tetrahydro-1,4-benzoxazepine. ¹H NMR (400 MHz, DMSO-d₆): δ 12.51 (s, 1H), 8.24 (s, 1H), 8.12-7.24 (m, 7H), 7.1-6.99 (m, 1H), 6.72-6.57 (m, 1H), 4.82 (s, 2H), 4.24 (s, 2H), 3.98 (s, 2H); MS (EI) for C₂₁H₁₇N₃O₃: 360.0 (MH+).

2-(4-{[7-(1H-Benzimidazol-6-yl)-2,3-dihydro-1,4-benzoxazepin-4(5H)-yl]carbonyl}phenyl)-2-methylpropanenitrile. ¹H NMR (400 MHz, CDCl₃): δ 10.6 (br s, 1H), 8.03 (s, 1H), 7.81 (br d, 1H), 7.68 (br d, 2H), 7.57 (br dd, 2H), 7.49-7.39 (m, 3H), 7.17 (d, 1H), 6.85 (d, 1H), 4.80 (br s, 0.2H), 4.42 (br s, 1.8H), 4.29-4.25 (m, 1.8H), 4.22-4.15 (m, 1.8H), 4.09 (br s, 0.2H), 3.85 (br s, 0.2H), 1.83 (s, 5.4H), 1.73 (s, 0.6H). MS (EI) for C₂₇H₂₄N₄O₂: 437.1 (MH⁺).

2-{[7-(1H-Benzimidazol-6-yl)-2,3-dihydro-1,4-benzoxazepin-4(5H)-yl]carbonyl}-N-ethyl-5-(methylsulfonyl)aniline. ¹H NMR (400 MHz, CDCl₃): δ 8.09 (br s, 1H), 7.80 (d, 1H), 7.52 (dd, 1H), 7.46-7.37 (m, 3H), 7.28-7.22 (m, 3H), 7.13 (d, 1H), 6.47 (br s, 1H), 4.46 (br s, 2H), 4.28-4.12 (m, 5H), 3.19 (s, 3H), 2.95 (br s, 2H), 0.99 (t, 3H). MS (EI) for C₂₆H₂₆N₄O₄S: 491.1 (MH⁺).

1-(4-{[7-(1H-Benzimidazol-6-yl)-2,3-dihydro-1,4-benzoxazepin-4(5H)-yl]carbonyl}-3-methylphenyl)-2,2,2-trifluoroethanone. ¹H NMR (400 MHz, DMSO-d₆): δ 12.6-12.3 (m, 1H), 8.28-8.21 (m, 1H), 7.97-7.65 (m, 2H), 7.64-7.42 (m, 4.5H), 7.25-7.03 (m, 2H), 6.52 (d, 0.5H), 4.90 (br d, 1H), 4.45-3.92 (m, 3H), 3.55 (br s, 1H), 3.33 (br s, 1H), 2.17 (s, 1.5H), 1.92-1.88 (m, 1.5H). MS (EI) for C₂₆H₂₀F₃N₃O₃: 480.0 (MH⁺).

1,1-Dimethylethyl {2-[(4-{[7-(1H-benzimidazol-6-yl)-2,3-dihydro-1,4-benzoxazepin-4(5H)-yl]carbonyl}phenyl)sulfonyl]ethyl}carbamate. ¹H NMR (400 MHz, CDCl₃): δ 8.12 (d, 2.5H), 7.97 (d, 0.5H), 7.83-7.69 (m, 1H), 7.61-7.50 (m, 3H), 7.42 (d, 1H), 7.30-7.21 (m, 1H), 7.15 (d, 1H), 6.37 (d, 1H), 5.24-5.15 (m, 0.85H), 4.91 (br s, 0.15H), 4.43 (s, 1.5H), 4.29-4.04 (m, 3.5H), 3.81-3.53 (m, 2H), 3.51-3.29 (m, 2H), 2.13 (br s, 1H), 1.45-1.24 (m, 9H). MS (EI) for C₃₀H₃₂N₄O₆S: 577.0 (MH⁺).

7-(1H-Benzimidazol-6-yl)-4-[(4-{[2-(tetrahydro-2H-pyran-2-yloxy)ethyl]sulfonyl}phenyl)carbonyl]-2,3,4,5-tetrahydro-1,4-benzoxazepine. ¹H NMR (400 MHz, CDCl₃): δ 8.15 (d, 2H), 8.09 (s, 1H), 7.97 (d, 0.25H), 7.79 (d, 1.5H), 7.57-7.47 (m, 3.25H), 7.42 (dd, 1H), 7.24 (br s, 1H), 7.14 (d, 1H), 6.35 (d, 1H), 4.90 (s, 0.25H), 4.54-4.50 (m, 1H), 4.41 (s, 2H), 4.26-4.12 (m, 4.5H), 4.11-4.03 (m, 0.25H), 3.90 (dt, 1H), 3.85-3.66 (m, 1H), 3.60 (t, 2H), 3.51-3.42 (m, 1H), 1.64-1.24 (m, 6H). MS (EI) for C₃₀H₃₁N₃O₆S: 562.0 (MH⁺).

2-[(4-{[7-(1H-Benzimidazol-6-yl)-2,3-dihydro-1,4-benzoxazepin-4(5H)-yl]carbonyl}phenyl)sulfonyl]ethanol. ¹H NMR (400 MHz, CDCl₃): δ 8.12 (d, 2H), 8.06 (s, 1H), 7.98 (d, 0.25H), 7.72 (d, 1.5H), 7.59-7.49 (m, 3.25H), 7.46 (dd, 1H), 7.36 (br s, 1H), 7.16 (d, 1H), 6.51 (d, 1H), 4.91 (s, 0.25H), 4.43 (s, 2H), 4.29-4.23 (m, 2H), 4.22-4.16 (m, 2H), 4.11 (t, 2H), 4.05-3.99 (m, 0.25H), 3.81-3.74 (m, 0.25H), 3.53 (t, 2H), 3.40-3.35 (m, 0.25H). MS (EI) for C₂₅H₂₃N₃O₅S: 478.0 (MH⁺).

7-(1H-Benzimidazol-6-yl)-4-[(2-ethynylphenyl)carbonyl]-2,3,4,5-tetrahydro-1,4-benzoxazepine. ¹H NMR (400 MHz, CDCl₃): δ 8.03 (d, 1H), 7.67-7.35 (m, 7H), 7.33-7.28 (m, 1H), 7.23-7.16 (m, 0.5H), 7.11 (d, 1H), 6.62 (d, 0.5H), 4.96 (br d, 1H), 4.52-3.92 (m, 4H), 3.73 (t, 1H), 2.98 (d, 1H). MS (EI) for C₂₅H₁₉N₃O₂: 394.1 (MH⁺).

1,1-Dimethylethyl [(4-{[7-(1H-Benzimidazol-6-yl)-2,3-dihydro-1,4-benzoxazepin-4(5H)-yl]carbonyl}phenyl)methyl]carbamate. ¹H NMR (400 MHz, CD₃OD): δ 8.20 (s, 1H), 7.82 (s, 0.5H), 7.71-7.62 (m, 2H), 7.58-7.49 (m, 1H), 7.45-7.25 (m, 5H), 7.14-7.07 (m, 1H), 6.84 (s, 0.5H), 4.68-4.56 (m, 2H), 4.35-4.21 (m, 3H), 4.16-4.06 (m, 2H), 3.90-3.83 (m, 1H), 1.48-1.26 (m, 9H). MS (EI) for C₂₉H₃₀N₄O₄: 499.2 (MH⁺).

7-(1H-Benzimidazol-6-yl)-4-{[2-chloro-4-(methylsulfonyl)-phenyl]carbonyl}-2,3,4,5-tetrahydro-1,4-benzoxazepine. ¹H NMR (400 MHz, CDCl₃): δ 8.25-8.15 (m, 2H), 8.08-8.00 (m, 1H), 7.85-7.75 (m, 1H), 7.55-7.36 (m, 3H), 7.24-7.15 (m, 2H), 6.37 (s, 1H), 4.47-3.80 (m, 6H), 3.24 (s, 3H). MS (EI) for C₂₄H₂₀ClN₃O₄S: 482 (M+H).

7-(1H-Benzimidazol-6-yl)-4-[(2,4-dimethylphenyl)carbonyl]-2,3,4,5-tetrahydro-1,4-benzoxazepine. ¹H NMR (400 MHz, CDCl₃): δ 8.12-7.98 (m, 1H), 7.71-7.42 (m, 4H), 7.35-7.31 (m, 1H), 7.15-7.05 (m, 4H), 6.64-6.61 (m, 1H), 5.10-4.80 (m, 1H), 4.47-3.95, (m, 4H), 3.73-3.68 (m, 1H), 2.37-2.00 (m, 6H). MS (EI) for C₂₅H₂₃CN₃O₂: 398 (M+H).

N-(2-Aminoethyl)-4-{[7-(1H-benzimidazol-6-yl)-2,3-dihydro-1,4-benzoxazepin-4(5H)-yl]carbonyl}benzenesulfonamide. ¹H NMR (400 MHz, DMSO-d₆): δ 9.64-9.60 (s, 1H), 8.35-6.88 (m, 13H), 4.94-4.12 (m, 4H), 3.75-3.35 (m, 5H), 3.04-2.98 (m, 2H), 2.90-2.80 (m, 2H). MS (EI) for C₂₅H₂₅N₅O₄S: 492 (M+H).

4-{[7-(1H-Benzimidazol-6-yl)-2,3-dihydro-1,4-benzoxazepin-4(5H)-yl]carbonyl}-N-methylbenzenesulfonamide. ¹H NMR (400 MHz, DMSO-d₆): δ 12.54-12.49 (s, 1H), 8.28 (s, 1H), 7.93-6.75 (m, 11H), 4.90-3.71 (m, 6H), 2.45-2.40 (m, 3H). MS (EI) for C₂₄H₂₂N₄O₄S: 463 (M+H).

4-{[7-(1H-Benzimidazol-6-yl)-2,3-dihydro-1,4-benzoxazepin-4(5H)-yl]carbonyl}-N-ethylbenzenesulfonamide. ¹H NMR (400 MHz, DMSO-d₆): δ 12.65-12.50 (br s, 1H), 12.0 (s, 1H), 8.29 (s, 1H), 7.90-6.77 (m, 11H), 4.90-3.70 (m, 6H), 2.84-2.72 (m, 2H), 1.95 (s, 3H), 1.02-0.84 (m, 3H). MS (EI) for C₂₅H₂₄N₄O₄S: 477 (M+H).

(4-{[7-(1H-Benzimidazol-6-yl)-2,3-dihydro-1,4-benzoxazepin-4(5H)-yl]carbonyl}phenyl)methanol. ¹H NMR (400 MHz, DMSO-d₆): δ 12.60-12.45 (br s, 1H), 8.37 (s, 1H), 7.93-6.80 (m, 8H), 5.60-5.13 (m, 1H), 5.00-3.60 (m, 9H), 1.88 (s, 1H). MS (EI) for C₂₄H₂ᵢN₃O₃: 400 (M+H).

7-(1H-Benzimidazol-6-yl)-4-[(1-methyl-1H-pyrrol-2-yl)carbonyl]-2,3,4,5-tetrahydro-1,4-benzoxazepine. ¹H NMR (400 MHz, DMSO-d₆): δ 12.02 (br s, 1H), 8.32 (s, 1H), 7.87-7.55 (m, 4H), 7.10-6.89 (m, 2H), 6.28 (s, 1H), 6.09-6.02 (m, 1H), 4.83 (s, 2H), 4.26 (m, 2H), 4.04 (m, 2H), 3.58 (s, 3H). MS (EI) for C₂₂H₂₀N₄O₂: 373 (M+H).

4-{[7-(1H-Benzimidazol-6-yl)-2,3-dihydro-1,4-benzoxazepin-4(5H)-yl]carbonyl}-N-(2-hydroxyethyl)benzenesulfonamide. ¹H NMR (400 MHz, DMSO-d₆): δ 8.26 (s, 1H), 8.00-6.70 (m, 10H), 4.88 (s, 1H), 4.53 (s, 1H), 4.33-4.00 (m, 4H), 3.42-3.31 (m, 2H), 2.87-2.77 (m, 2H), 1.92 (s, 3H). MS (EI) for C₂₅H₂₄N₄O₅S: 493 (M+H).

7-(1H-Benzimidazol-6-yl)-4-{[4-methyl-3-(methyloxy)phenyl]-carbonyl}-2,3,4,5-tetrahydro-1,4-benzoxazepine. ¹H NMR (400 MHz, DMSO-d₆): δ 8.25 (s, 1H), 7.93-6.62 (m, 9H), 4.83 (s, 1H), 4.60 (s, 1H), 4.43-3.44 (m, 7H), 2.18 (s, 3H), 1.92 (s, 3H). MS (EI) for C₂₅H₂₃N₃O₃: 414 (M+H).

N-(4-{[7-(1H-Benzimidazol-6-yl)-2,3-dihydro-1,4-benzoxazepin-4(5H)-yl]carbonyl}phenyl)-N-methylmethanesulfonamide. ¹H NMR (400 MHz, DMSO-d₆): δ 12.56 (br s, 1H), 8.26 (s, 1H), 7.99-6.72 (m, 10H), 4.97-3.66 (m, 6H), 3.27 (s, 3H), 2.98 (s, 3H). MS (EI) for C₂₅H₂₄N₄O₄S: 477 (M+H).

N-(4-{[7-(1H-Benzimidazol-6-yl)-2,3-dihydro-1,4-benzoxazepin-4(5H)-yl]carbonyl}phenyl)ethanesulfonamide. ¹H NMR (400 MHz, DMSO-d₆): δ 8.26 (s, 1H), 7.90-6.82 (m, 10H), 4.82 (s, 1H), 4.57 (s, 1H), 4.37-3.72 (m, 5H), 3.19-3.06 (m, 2H), 1.30-1.09 (m, 3H). MS (EI) for C₂₅H₂₄N₄O₄S: 477 (M+H).

7-(1H-Benzimidazol-6-yl)-4-[(1-ethyl-3-methyl-1H-pyrazol-5-yl)carbonyl]-2,3,4,5-tetrahydro-1,4-benzoxazepine. ¹H NMR (400 MHz, DMSO-d₆): δ 8.26 (s, 1H), 7.90-6.98 (m, 6H), 6.32-6.09 (m, 1H), 4.95-4.64 (m, 2H), 4.38-3.77 (m, 6H), 2.31-2.09 (m, 3H), 1.89 (s, 3H), 1.30-0.93 (m, 3H). MS (EI) for C₂₃H₂₃N₅O₂: 402 (M+H).

7-(1H-Benzimidazol-6-yl)-4-[(2-bromo-4-chlorophenyl)-carbonyl]-2,3,4,5-tetrahydro-1,4-benzoxazepine. ¹H NMR (400 MHz, DMSO-d₆): δ 12.58 (br s, 1H), 8.25 (d, 1H), 7.99-6.75 (m, 9H), 5.05-3.91 (m, 5H), 3.68-3.47 (m, 1H). MS (EI) for C₂₃H₁₇BrClN₃O₂: 482 (M+H).

7-(1H-Benzimidazol-6-yl)-4-[(1-methyl-1H-benzimidazol-2-yl)carbonyl]-2,3,4,5-tetrahydro-1,4-benzoxazepine. ¹H NMR (400 MHz, DMSO-d₆): δ 12.64-12.39 (m, 1H), 8.24 (m, 1H), 8.05-6.85 (m, 10H), 4.98-4.83 (m, 2H), 4.34-4.08 (m, 4H), 3.84-3.56 (m, 3H). MS (EI) for C₂₅H₂₁N₅O₂: 424 (M+H).

7-(1H-Benzimidazol-6-yl)-4-{[4-(propylsulfonyl)-phenyl]carbonyl}-2,3,4,5-tetrahydro-1,4-benzoxazepine. ¹H NMR (400 MHz, DMSO-d₆): δ 9.63-9.47 (m, 2H), 8.11-6.83 (m, 11H), 5.00-3.68 (m, 6H), 1.63-1.43 (m, 2H), 1.03-0.76 (m, 3H). MS (EI) for C₂₆H₂₅N₃O₄S: 476 (M+H).

7-(1H-Benzimidazol-6-yl)-4-{[4-(ethylsulfonyl)-phenyl]carbonyl}-2,3,4,5-tetrahydro-1,4-benzoxazepine). ¹H NMR (400 MHz, CDCl₃): δ 8.15-8.08 (m, 3H), 7.84-7.70 (m, 1H), 7.60-7.49 (m, 4H), 7.45-7.40 (m, 1H), 7.23 (s, 1H), 7.17-7.12 (m, 1H), 6.34 (s, 1H), 4.40 (s, 2H), 4.28-4.05 (m, 4H), 3.34-3.07 (m, 2H), 1.43-1.21 (m, 3H); MS (EI) for C₂₅H₂₃N₃O₄S: 462 (MH⁺).

7-(1H-Benzimidazol-6-yl)-4-{[4-(methylsulfonyl)naphthalen-1-yl]carbonyl}-2,3,4,5-tetrahydro-1,4-benzoxazepine. ¹H NMR (400 MHz, d₆-DMSO): δ 12.58 (m, 1H), 8.75-8.63 (m, 1H), 8.30-8.14 (m, 2H), 7.96-6.20 (m, 10H), 5.15-4.88 (m, 1H), 4.46-3.97 (m, 4H), 3.56-3.49 (m, 1H), 2.87 (s, ˜1H), 2.71 (s, ˜1H), 2.66 (s, 1H); MS (EI) for C₂₈H₂₃N₃O₂S: 496 (M−H).

2-(4-{[7-(1H-Benzimidazol-6-yl)-2,3-dihydro-1,4-benzoxazepin-4(5H)-yl]carbonyl}phenyl)ethanamine dihydrochloride. ¹H NMR (400 MHz, CDCl₃): δ 8.06 (s, 1H), 7.89-7.29 (m, 9H), 7.25-7.11 (m, 1H), 6.77 (s, 1H), 5.02-4.82 (m, 1H), 4.64-4.40 (m, 2H), 4.32-4.04 (m, 4H), 3.58-3.35 (m, 2H), 3.00-2.77 (m, 2H), 1.54 (s, 9H); MS (EI) for C₃₀H₃₂N₄O₄: 513 (MH⁺).

2-(4-{[7-(1H-Benzimidazol-6-yl)-2,3-dihydro-1,4-benzoxazepin-4(5H)-yl]carbonyl}phenyl)ethanamine. ¹H NMR (400 MHz, CDCl₃): δ 9.51 (s, 1H), 8.20-7.62 (m, 7H), 7.44-7.00 (m, 4H), 4.92-4.47 (m, 2H), 4.38-4.10 (m, 2H), 4.06-3.72 (m, 2H), 3.45 (br s, 2H), 3.16-2.86 (m, 4H); MS (EI) for C₂₅H₂₄N₄O₂: 413 (MH⁺).

2-[(4-{[7-(1H-Benzimidazol-6-yl)-2,3-dihydro-1,4-benzoxazepin-4(5H)-yl]carbonyl}phenyl)oxy]ethanamine dihydrochloride. ¹H NMR (400 MHz, CDCl₃): δ 9.52 (s, 1H), 8.26 (br s, 3H), 8.06-7.65 (m, 3H), 7.64-7.56 (m, 1H), 7.51-7.21 (m, 2H), 7.20-6.99 (m, 2H), 5.02-4.51 (m, 2H), 4.39-4.12 (m, 4H), 4.06-3.77 (m, 2H), 3.46 (br s, 2H), 3.28-3.16 (m, 2H); MS (EI) for C₂₅H₂₄N₄O₃: 429 (MH⁺).

7-(1H-Benzimidazol-6-yl)-4-[(1-methyl-1H-indol-2-yl)carbonyl]-2,3,4,5-tetrahydro-1,4-benzoxazepine. ¹H NMR (400 MHz, DMSO-d₆): δ 12.2 (d, 1H), 8.18 (s, 1H), 7.97-7.53 (m, 6H), 7.32-6.52 (m, 5H), 4.84 (m, 2H), 4.25 (m, 2H), 4.05 (s, 3H), 3.60 (m, 2H); MS (EI) for C₂₆H₂₂N₄O₂: 423.2 (MH⁺).

4-{[7-(1H-Benzimidazol-6-yl)-2,3-dihydro-1,4-benzoxazepin-4(5H)-yl]carbonyl}-N,N-dimethylbenzenesulfonamide. ¹H NMR (400 MHz, DMSO-d₆): δ 12.2 (bs, 1H), 8.09 (s, 1H), 7.95-7.48 (m, 8H), 7.35-6.75 (m, 2H), 4.85 (s, 1H), 4.51 (s, 1H), 4.12-4.05 (m, 3H), 3.71 (s, 1H), 2.61 (s, 3H), 2.53 (s, 3H); MS (EI) for C₂₅H₂₄N₄O₄S: 477.2 (MH⁺).

7-(1H-Benzimidazol-6-yl)-4-{[4-(piperidin-1-ylsulfonyl)-phenyl]carbonyl}-2,3,4,5-tetrahydro-1,4-benzoxazepine. ¹H NMR (400 MHz, DMSO-d₆): δ 12.2 (bs, 1H), 8.23 (s, 1H), 7.81-7.45 (m, 8H), 7.24-6.81 (m, 2H), 4.88 (s, 1H), 4.50 (s, 1H), 4.28-4.05 (m, 3H), 3.72 (s, 1H), 2.89 (m, 2H), 2.85 (m, 2H), 1.54 (m, 2H), 1.41 (m, 3H), 1.22 (m, 1H); MS (EI) for C₂₈H₂₈N₄O₄S: 517.2 (MH⁺).

1-(4-{[7-(1H-Benzimidazol-6-yl)-2,3-dihydro-1,4-benzoxazepin-4(5H)-yl]carbonyl}phenyl)methanamine dihydrochloride salt. ¹H NMR (400 MHz, d₆-DMSO): δ 9.62-9.59 (m, 1H), 8.62-8.43 (m, 3H), 8.04 (s, 0.5H), 7.99-7.84 (m, 2H), 7.80-7.52 (m, 4H), 7.49-7.42 (m, 1H), 7.39-7.32 (m, 1H), 7.20-7.08 (m, 1H), 7.00 (s, 0.5H), 4.89 (br s, 1H), 4.56 (br s, 1H), 4.32 (br s, 1H), 4.19 (br s, 2H), 4.11-4.00 (m, 2H), 3.77 (br s, 1H). MS (EI) for C₂₄H₂₂N₄O₂: 399.1 (MH⁺).

2-[(4-{[7-(1H-Benzimidazol-6-yl)-2,3-dihydro-1,4-benzoxazepin-4(5H)-yl]carbonyl}phenyl)sulfonyl]ethanamine dihydrochloride salt. ¹H NMR (400 MHz, d₆-DMSO): δ 9.65-9.60 (m, 1H), 8.35-8.22 (m, 3H), 8.07-7.98 (m, 2.5H), 7.97-7.92 (m, 1H), 7.92-7.86 (m, 1H), 7.79 (br s, 0.5H), 7.72 (d, 1H), 7.67-7.60 (m, 1H), 7.56 (d, 0.5H), 7.18-7.11 (m, 1H), 7.03 (br s, 0.5H), 4.93 (br s, 2H), 4.68 (br s, 1H), 4.34 (br s, 1H), 4.20 (br s, 2H), 4.06 (br s, 1H), 3.80-3.70 (m, 2H), 3.11-3.00 (m, 2H). MS (EI) for C₂₅H₂₄N₄O₄S: 477.0 (MH⁺).

7-(1H-Benzimidazol-6-yl)-4-{[4-(methylsulfonyl)-2-propylphenyl]carbonyl}-2,3,4,5-tetrahydro-1,4-benzoxazepine. ¹H NMR (400 MHz, CDCl₃): δ 8.11 (br s, 1H), 8.01-7.96 (m, 2H), 7.81 (d, 1H), 7.54 (dd, 1H), 7.43 (dd, 1H), 7.31 (d, 1H), 7.29-7.26 (m, 1H), 7.15 (d, 1H), 6.29 (d, 1H), 4.38 (d, 1H), 4.28-4.21 (m, 5H), 3.20 (s, 3H), 2.46-2.35 (m, 1H), 2.26-2.15 (m, 1H), 1.74-1.57 (m, 1H), 1.55-1.39 (m, 1H), 0.86 (t, 3H). MS (EI) for C₂₇H₂₇N₃O₄S: 490.1 (MH⁺).

7-(1H-Benzimidazol-6-yl)-4-{[2-ethyl-4-(methylsulfonyl)-phenyl]carbonyl}-2,3,4,5-tetrahydro-1,4-benzoxazepine. ¹H NMR (400 MHz, CDCl₃): δ 8.10 (br s, 1H), 8.04-8.01 (m, 1H), 7.99 (dd, 1H), 7.80 (d, 1H), 7.53 (dd, 1H), 7.44 (dd, 1H), 7.33 (d, 1H), 7.28-7.25 (m, 1H), 7.15 (d, 1H), 6.29 (d, 1H), 4.38 (d, 1H), 4.30-4.17 (m, 5H), 3.21 (s, 3H), 2.55-2.43 (m, 1H), 2.30-2.18 (m, 1H), 1.14 (t, 3H). MS (EI) for C₂₆H₂₅N₃O₄S: 476.0 (MH+).

4-{[7-(1H-Benzimidazol-6-yl)-2,3-dihydro-1,4-benzoxazepin-4(5H)-yl]carbonyl}-3-ethyl-N-(1-methylethyl)benzenesulfonamide. ¹H NMR (400 MHz, DMSO-d₆): δ 12.55 (s, 1H), 8.31-8.20 (s, 1H), 8.05-6.47 (m, 9H), 5.10-4.76 (m, 1H), 4.53-3.93 (m, 5H), 2.72-2.11 (m, 2H), 1.18-0.75 (m, 9H); MS (EI) for C₂₈H₃₀N₄O₄S: 519.2 (MH+).

4-{[7-(1H-Benzimidazol-6-yl)-2,3-dihydro-1,4-benzoxazepin-4(5H)-yl]carbonyl}-3-bromo-N-(1-methylethyl)benzenesulfonamide. ¹H NMR (400 MHz, DMSO-d₆): δ 12.51 (s, 1H), 8.25 (s, 1H), 8.11-7.99 (m, 1H), 7.91-7.78 (m, 2H), 7.75-6.59 (m, 6H), 5.05-4.73 (m, 1H), 4.53-3.96 (m, 5H), 1.03-0.79 (m, 6H); MS (EI) for C₂₆H₂₅BrN₄O₄S: 569.1 (M+).

7-(1H-Benzimidazol-6-yl)-4-[(4-ethyl-2-methyl-4H-thieno[3,2-b]pyrrol-5-yl)carbonyl]-2,3,4,5-tetrahydro-1,4-benzoxazepine. ¹H NMR (400 MHz, DMSO-d₆): δ 12.50 (m, 1H), 8.24 (s, 1H), 7.88-7.27 (m, 5H), 7.11-6.90 (m, 2H), 6.57 (s, 1H), 4.87 (s, 2H), 4.32-4.22 (m, 2H), 4.15-3.99 (m, 4H), 1.10 (m, 3H); MS (EI) for C₂₆H₂₄N₄O₂S: 457.2 (MH+).

7-(1H-Benzimidazol-6-yl)-4-{[3-fluoro-2-methyl-4-(methylsulfonyl)phenyl]carbonyl}-2,3,4,5-tetrahydro-1,4-benzoxazepine. ¹H NMR (400 MHz, DMSO-d₆): δ 12.49 (s, 1H), 8.25 (m, 1H), 7.89-7.18 (m, 7H), 7.13-6.73 (m, 1H), 4.92 (m, 1H), 4.58-3.89 (m, 4H), 3.68-3.49 (m, 1H), 2.13-1.78 (m, 3H); MS (EI) for C₂₅H₂₂FN₃O₄S: 480.0 (MH+).

7-(1H-Benzimidazol-6-yl)-4-{[2-ethyl-3-fluoro-4-(methylsulfonyl)phenyl]carbonyl}-2,3,4,5-tetrahydro-1,4-benzoxazepine. ¹H NMR (400 MHz, DMSO-d₆): δ 12.61-12.33 (m, 1H), 8.25 (m, 1H), 7.99-7.46 (m, 5H), 7.35-6.74 (m, 3H), 5.09-4.74 (m, 1H), 4.54-3.93 (m, 4H), 3.70-3.51 (m, 1H), 2.75-2.30 (m, 2H), 1.16-0.91 (m, 3H); MS (EI) for C₂₆H₂₄FN₃O₄S: 494.2 (MH+).

7-(1H-Benzimidazol-6-yl)-4-{[2-ethyl-4-(ethylsulfonyl)-3-fluorophenyl]carbonyl}-2,3,4,5-tetrahydro-1,4-benzoxazepine. ¹H NMR (400 MHz, DMSO-d₆): δ 12.61-12.37 (m, 1H), 8.24 (m, 1H), 7.96-7.42 (m, 5H), 7.35-6.65 (m, 3H), 5.08-4.76 (m, 1H), 4.52-3.97 (m, 4H), 3.67-3.55 (m, 1H), 3.48-3.38 (m, 2H), 2.72-2.42 (m, 2H), 1.29-0.92 (m, 6H); MS (EI) for C₂₇H₂₆FN₃O₄S: 508.2 (MH+).

7-(1H-Benzimidazol-6-yl)-4-{[2-ethyl-4-(1,3-thiazol-2-yl)phenyl]carbonyl}-2,3,4,5-tetrahydro-1,4-benzoxazepine. ¹H NMR (400 MHz, DMSO-d₆): δ 12.59-12.40 (m, 1H), 8.28-8.20 (m, 1H), 8.03-7.41 (m, 8H), 7.32-6.62 (m, 3H), 5.09-4.77 (m, 1H), 4.54-4.01 (m, 4H), 3.67-3.56 (m, 1H), 2.72-2.22 (m, 2H), 1.19-0.94 (m, 6H); MS (EI) for C₂₈H₂₄N₄O₂S: 480.9 (MH+).

4-{[4-(Ethylsulfonyl)phenyl]carbonyl}-7-(2-methyl-1H-benzimidazol-6-yl)-2,3,4,5-tetrahydro-1,4-benzoxazepine hydrochloride. ¹H NMR (400 MHz, DMSO-d₆): δ 7.98-7.89 (m, 2.5H), 7.87-7.73 (m, 2.5H), 7.70-7.47 (m, 3H), 7.16-7.09 (m, 0.5H), 6.87 (s, 0.5H), 4.96-4.51 (m, 2H), 4.36-3.69 (m, 4H), 3.39-3.28 (m, 2H), 2.86-2.78 (m, 3H), 1.15-0.99 (m, 3H). MS (EI) for C₂₆H₂₅N₃O₄S: 476 (M+H).

7-(2-Methyl-1H-benzimidazol-6-yl)-4-({4-[(1-methylethyl)-sulfonyl]phenyl}carbonyl)-2,3,4,5-tetrahydro-1,4-benzoxazepine. ¹H NMR (400 MHz, DMSO-d₆): δ 7.99-7.47 (m, 9H), 7.31-6.81 (m, 2H), 4.92 (s, 1.2H), 4.55 (s, 0.8H), 4.34-4.29 (m, 0.8H), 4.22-4.16 (m, 1.2H), 4.09-4.03 (m, 0.8H), 3.77-3.71 (m, 1.2H), 2.83-2.78 (m, 3H), 1.20-1.07 (m, 6H). MS (EI) for C₂₇H₂₇N₃O₄S: 490 (M+H).

4-[(4-Chloro-2-ethylphenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.97 (s, 0.5H), 7.85-7.75 (m, 2.5H), 7.64-7.52 (m, 1.5H), 7.43-7.28 (m, 2H), 7.20-7.02 (m, 2H), 6.79 (d, 0.5H), 5.05-4.80 (m, 1H), 4.54-4.04 (m, 4H), 3.60-3.55 (m, 1H), 2.48-2.17 (m, 2H), 1.09 (t, 1.5H), 0.97 (t, 1.5H). MS (EI) for C₂₆H₂₄ClN₃O₂: 446 (M+H).

4-(Biphenyl-4-ylcarbonyl)-7-(2-methyl-1H-benzimidazol-6-yl)-2,3,4,5-tetrahydro-1,4-benzoxazepine. ¹H NMR (400 MHz, DMSO-d₆): δ 8.00-7.34 (m, 13.5H), 7.16-6.99 (m, 1.5H), 4.90 (br s, 1.3H), 4.66 (br s, 0.7H), 4.37-3.80 (m, 4H), 2.83-2.74 (m, 3H). MS (EI) for C₃₀H₂₅N₃O₂: 460 (M+H).

4-[(2-Ethyl-4-iodophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.96 (s, 0.5H), 7.85-7.49 (m, 6H), 7.13-7.08 (m, 1H), 6.93 (d, 0.5H), 6.81 (d, 0.5H), 6.73 (d, 0.5H), 5.04-4.79 (m, 1H), 4.53-4.03 (m, 4H), 3.60-3.54 (m, 1H), 2.82-2.79 (m, 3H), 2.48-2.13 (m, 2H), 1.07 (t, 1.7H), 0.94 (t, 1.3H). MS (EI) for C₂₆H₂₄IN₃O₂: 538 (M+H).

4-[(4-Bromo-2-ethylphenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.96 (s, 0.5H), 7.86-7.75 (m, 2.5H), 7.64-7.50 (m, 2.5H), 7.46-6.77 (m, 3.5H), 5.03-4.81 (m, 1H), 4.53-4.04 (m, 4H), 3.60-3.54 (m, 1H), 2.83-2.79 (m, 3H), 2.47-2.19 (m, 2H), 1.08 (t, 1.7H), 0.96 (t, 1.3H). MS (EI) for C₂₆H₂₄BrN₃O₂: 490 (M+H).

4-[(4,5-Dibromo-2-ethylphenyl)carbonyl]-7-(2-methyl-1H-benzimidazol-6-yl)-2,3,4,5-tetrahydro-1,4-benzoxazepine. ¹H NMR (400 MHz, DMSO-d₆): δ 12.23 (br s, 1H), 7.75-7.37 (m, 5.5H), 7.32 (s, 0.5H), 7.21 (d, 0.5H), 7.09-7.03 (m, 1H), 6.85 (d, 0.5H), 4.95-4.79 (m, 1H), 4.55-3.90 (m, 4H), 3.60-3.57 (m, 1H), 2.47-2.23 (m, 2H), 1.07 (t, 1.3H), 0.99 (t, 1.7H). MS (EI) for C₂₆H₂₄Br₂N₃O₂: 568 (M+H).

4-{[2-Bromo-4-(trifluoromethyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 8.13 (s, 1H), 7.97 (s, 0.5H), 7.86-7.70 (m, 3.5H), 7.66-7.37 (m, 2.5H), 7.15-7.10 (m, 1H), 6.79 (d, 0.5H), 5.04-4.83 (m, 1H), 4.56-3.99 (m, 4H), 3.64-3.52 (m, 1H), 2.83-2.77 (m, 3H). MS (EI) for C₂₅H₁₉BrF₃N₃O₂: 530 (M+H).

4-{[2-Ethyl-4-(trifluoromethyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 7.95 (s, 0.5H), 7.84-7.53 (m, 6H), 7.46-7.07 (m, 2H), 6.66 (d, 0.5H), 5.06-4.81 (m, 1H), 4.50-4.04 (m, 4H), 3.57-3.52 (m, 1H), 2.81-2.76 (m, 3H), 2.63-2.24 (m, 2H), 1.10 (t, 1.7H), 0.97 (t, 1.3H). MS (EI) for C₂₇H₂₄F₃N₃O₂: 480 (M+H).

7-(2-Methyl-1H-benzimidazol-6-yl)-4-({4-[(2-methylpropyl)-sulfonyl]phenyl}carbonyl)-2,3,4,5-tetrahydro-1,4-benzoxazepine. ¹H NMR (400 MHz, DMSO-d₆): δ 7.99-7.94 (m, 2.5H), 7.87-7.74 (m, 2.5H), 7.68-7.48 (m, 3.5H), 7.16-7.09 (m, 1H), 6.87 (s, 0.5H), 4.91 (s, 1.3H), 4.55 (s, 0.7H), 4.35-4.30 (m, 0.7H), 4.21-4.16 (m, 1.3H), 4.08-4.03 (m, 0.7H), 3.76-3.70 (m, 1.3H), 3.29-3.23 (m, 2H), 2.83-2.77 (m, 3H), 2.07-1.94 (m, 1H), 1.01-0.88 (m, 6H). MS (EI) for C₂₈H₂₉N₃O₄S: 504 (M+H).

7-(2-Methyl-1H-benzimidazol-6-yl)-4-[(7-methyl-1H-indol-2-yl)carbonyl]-2,3,4,5-tetrahydro-1,4-benzoxazepine. ¹H NMR (400 MHz, DMSO-d₆): δ 11.47 (s, 1H), 7.96-7.57 (m, 5H), 7.42 (d, 1H), 7.09 (d, 1H), 6.99-6.94 (m, 2H), 6.79 (br s, 1H), 4.98 (br s, 2H), 4.40-4.16 (m, 4H), 2.80 (s, 3H), 2.45 (s, 3H). MS (EI) for C₂₇H₂₄N₄O₂: 437 (M+H).

7-(2-Methyl-1H-benzimidazol-6-yl)-4-{[7-(methyloxy)-1H-indol-2-yl]carbonyl}-2,3,4,5-tetrahydro-1,4-benzoxazepine. ¹H NMR (400 MHz, DMSO-d₆): δ 11.51 (s, 1H), 7.98-7.57 (m, 5H), 7.18-7.06 (m, 2H), 6.97 (t, 1H), 6.73 (d, 2H), 4.92 (s, 2H), 4.32 (br s, 2H), 4.14 (br s, 2H), 3.87 (s, 3H), 2.80 (s, 3H). MS (EI) for C₂₇H₂₄N₄O₃: 453 (M+H).

7-(2-Methyl-1H-benzimidazol-6-yl)-4-{[3-methyl-7-(methyloxy)-1H-indol-2-yl]carbonyl}-2,3,4,5-tetrahydro-1,4-benzoxazepine. ¹H NMR (400 MHz, DMSO-d₆): δ 11.25 (s, 1H), 8.01-7.56 (m, 5H), 7.11 (d, 2H), 6.97 (t, 1H), 6.71 (d, 1H), 4.95-4.50 (m, 2H), 4.26-3.74 (m, 7H), 2.79 (s, 3H), 2.24-1.92 (m, 3H). MS (EI) for C₂₈H₂₆N₄O₃: 467 (M+H).

4-{[5-Fluoro-7-(methylsulfonyl)-1H-indol-2-yl]carbonyl}-7-(2-methyl-1H-benzimidazol-6-yl)-2,3,4,5-tetrahydro-1,4-benzoxazepine. ¹H NMR (400 MHz, DMSO-d₆): δ 11.47 (s, 1H), 7.97-7.23 (m, 5H), 7.65-7.54 (m, 2H), 7.40-6.84 (m, 2H), 4.95 (s, 2H), 4.43-4.26 (m, 2H), 4.17-4.03 (m, 2H), 2.81 (s, 3H). MS (EI) for C₂₇H₂₃FN₄O₄S: 519 (M+H).

4-[(4-{[2,5-Bis(methyloxy)phenyl]sulfonyl}-2-methylphenyl)-carbonyl]-7-(2-methyl-1H-benzimidazol-6-yl)-2,3,4,5-tetrahydro-1,4-benzoxazepine. ¹H NMR (400 MHz, DMSO-d₆): δ 7.95 (s, 0.5H), 7.84-7.72 (m, 4.5H), 7.63-7.55 (m, 1H), 7.49 (d, 1H), 7.43-7.35 (m, 1H), 7.30-7.19 (m, 1.5H), 7.16-7.09 (m, 1.5H), 7.02 (d, 0.5H), 6.63 (d, 0.5H), 5.03-4.83 (m, 1H), 4.46-4.01 (m, 4H), 3.82-3.79 (m, 3H), 3.67 (s, 1.5H), 3.56-3.49 (m, 2.5H), 2.79 (s, 3H), 2.21 (s, 1.5H), 1.97 (s, 1.5H). MS (EI) for C₃₃H₃ᵢN₃O₆S: 598 (M+H).

7-(2-Methyl-1H-benzimidazol-6-yl)-4-[(6-phenyl-1H-indol-2-yl)carbonyl]-2,3,4,5-tetrahydro-1,4-benzoxazepine. ¹H NMR (400 MHz, DMSO-d₆): δ 11.70 (s, 1H), 7.94 (s, 1H), 7.86-7.57 (m, 8H), 7.47 (t, 2H), 7.39-7.32 (m, 2H), 7.10 (d, 1H), 6.88 (s, 1H), 5.01 (br s, 2H), 4.43-4.04 (m, 4H), 2.81 (s, 3H). MS (EI) for C₃₂H₂₆N₄O₂: 499 (M+H).

4-{[1-Ethyl-7-(methyloxy)-1H-indol-2-yl]carbonyl}-7-(2-methyl-1H-benzimidazol-6-yl)-2,3,4,5-tetrahydro-1,4-benzoxazepine. ¹H NMR (400 MHz, DMSO-d₆): δ 8.03-7.39 (m, 5H), 7.20-6.96 (m, 3H), 6.79-6.40 (m, 2H), 4.99-4.72 (m, 2H), 4.40-3.78 (br m, 9H), 2.79 (s, 3H), 1.22-1.00 (br m, 3H). MS (EI) for C₂₉H₂₈N₄O₃: 481 (M+H).

4-[(1-Ethyl-7-fluoro-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.02-7.37 (m, 6H), 7.18-7.01 (m, 3H), 6.80-6.52 (m, 1H), 5.01-4.78 (m, 2H), 4.41-3.96 (m, 6H), 2.83-2.76 (m, 3H), 1.33-1.08 (m, 3H). MS (EI) for C₂₈H₂₅FN₄O₂: 469 (M+H).

7-(2-Methyl-1H-benzimidazol-6-yl)-4-[(2-methyl-4-{[2-(methyloxy)phenyl]sulfonyl}phenyl)carbonyl]-2,3,4,5-tetrahydro-1,4-benzoxazepine. ¹H NMR (400 MHz, DMSO-d₆): δ 8.03-7.95 (m, 1.5H), 7.85-7.55 (m, 6.5H), 7.41-7.35 (m, 1H), 7.28 (d, 0.5H), 7.22-7.05 (m, 3H), 6.63 (d, 0.5H), 5.03-4.84 (m, 1H), 4.50-4.00 (m, 4H), 3.76 (s, 1.5H), 3.61 (s, 1.5H), 3.58-3.49 (m, 1H), 2.83-2.78 (m, 3H), 2.21 (s, 1.5H), 1.97 (s, 1.5H). MS (EI) for C₃₂H₂₉N₃O₅S: 568 (M+H).

7-(2-Methyl-1H-benzimidazol-6-yl)-4-[(2-methyl-4-{[4-(methyloxy)phenyl]sulfonyl}phenyl)carbonyl]-2,3,4,5-tetrahydro-1,4-benzoxazepine. ¹H NMR (400 MHz, DMSO-d₆): δ 7.97-7.74 (m, 7H), 7.63-7.55 (m, 1H), 7.43-7.36 (m, 1H), 7.27 (d, 0.5H), 7.17-7.05 (m, 3H), 6.62 (d, 0.5H), 5.02-4.82 (m, 1H), 4.47-3.99 (m, 4H), 3.83 (s, 1.8H), 3.79 (s, 1.2H), 3.55-3.48 (m, 1H), 2.84-2.79 (m, 3H), 2.20 (s, 1.7H), 1.95 (s, 1.3H). MS (EI) for C₃₂H₂₉N₃O₅S: 568 (M+H).

4-({4-[(2-Fluorophenyl)sulfonyl]-2-methylphenyl}carbonyl)-7-(2-methyl-1H-benzimidazol-6-yl)-2,3,4,5-tetrahydro-1,4-benzoxazepine. ¹H NMR (400 MHz, DMSO-d₆): δ 8.11-8.04 (m, 1H), 7.96 (s, 0.5H), 7.89-7.73 (m, 5.5H), 7.64-7.32 (m, 4.5H), 7.12 (dd, 1H), 6.64 (d, 0.5H), 5.03-4.85 (m, 1H), 4.46-3.99 (m, 4H), 3.57-3.50 (m, 1H), 2.83-2.79 (m, 3H), 2.22 (s, 1.7H), 1.97 (s, 1.3H). MS (EI) for C₃₁H₂₆FN₃O₄S: 556 (M+H).

4-({4-[(4-Fluorophenyl)sulfonyl]-2-methylphenyl}carbonyl)-7-(2-methyl-1H-benzimidazol-6-yl)-2,3,4,5-tetrahydro-1,4-benzoxazepine. ¹H NMR (400 MHz, DMSO-d₆): δ 8.10-6.60 (m, 13H), 5.00-4.85 (m, 1H), 4.46-4.24 (m, 2H), 4.18-4.00 (m, 2H), 3.52 (br s, 1H), 2.85 (s, 1.5H), 2.83 (s, 1.5H), 2.21 (s, 1.5H), 1.97 (s, 1.5H). MS (EI) for C₃₁H₂₆FN₃O₄S: 556 (M+H).

N-Methyl-N-(4-{[7-(2-methyl-1H-benzimidazol-6-yl)-2,3-dihydro-1,4-benzoxazepin-4(5H)-yl]carbonyl}phenyl)benzenesulfonamide. ¹H NMR (400 MHz, DMSO-d₆): δ 7.99-6.89 (m, 15H), 4.88 (br s, 1.2H), 4.55 (br s, 0.8H), 4.34-4.17 (m, 2H), 4.02 (br s, 0.9H), 3.76 (br s, 1.1H), 3.16 (s, 3H), 2.81 (s, 3H). MS (EI) for C₃₁H₂₈N₄O₄S: 553 (M+H).

N-Methyl-N-(3-methyl-4-{[7-(2-methyl-1H-benzimidazol-6-yl)-2,3-dihydro-1,4-benzoxazepin-4(5H)-yl]carbonyl}phenyl)methanesulfonamide. ¹H NMR (400 MHz, DMSO-d₆): δ 7.97 (s, 0.5H), 7.86-7.75 (m, 2.5H), 7.64-7.50 (m, 1.5H), 7.33-7.09 (m, 4H), 6.68 (d, 0.5H), 4.92 (br s, 1H), 4.47-3.98 (m, 4H), 3.59 (br s, 1H), 3.25-3.21 (m, 3H), 2.97 (s, 1.5H), 2.92 (s, 1.5H), 2.84-2.80 (m, 3H), 2.15 (s, 1.5H), 1.90 (s, 1.5H). MS (EI) for C₂₇H₂₈N₄O₄S: 505 (M+H).

4-[(1-Ethyl-4-fluoro-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.05-7.38 (m, 6H), 7.27-7.08 (m, 2H), 6.89 (dd, 1H), 6.82-6.48 (m, 1H), 5.01-4.84 (m, 2H), 4.44-4.00 (m, 6H), 2.81 (s, 3H), 1.23-1.07 (m, 3H). MS (EI) for C₂₈H₂₅N₄O₂: 469 (M+H).

7-(2-Methyl-1H-benzimidazol-6-yl)-4-[(4-{[4-(methyloxy)-phenyl]sulfonyl}phenyl)carbonyl]-2,3,4,5-tetrahydro-1,4-benzoxazepine. ¹H NMR (400 MHz, DMSO-d₆): δ 8.01-7.73 (m, 8H), 7.64-7.42 (m, 3H), 7.20-7.07 (m, 3H), 4.88 (s, 1.3H), 4.53 (s, 0.7H), 4.31 (br s, 0.7H), 4.15 (br s, 1.3H), 4.02 (br s, 0.7H), 3.86-3.78 (m, 3H), 3.69 (br s, 1.3H), 2.81 (s, 3H). MS (EI) for C₃₁H₂₇N₃O₅S: 554 (M+H).

4-[(4-{[5-Fluoro-2-(methyloxy)phenyl]-sulfonyl}phenyl)carbonyl]-7-(2-methyl-1H-benzimidazol-6-yl)-2,3,4,5-tetrahydro-1,4-benzoxazepine. ¹H NMR (400 MHz, DMSO-d₆): δ 8.00-7.94 (m, 2.5H), 7.85-7.74 (m, 3.5H), 7.65-7.44 (m, 4.5H), 7.27-7.21 (m, 0.5), 7.16-7.09 (m, 1.5H), 6.88 (s, 0.5H), 4.90 (s, 1.2H), 4.55 (s, 0.8H), 4.34-4.29 (m, 0.8H), 4.20-4.15 (m, 1.2H), 4.07-4.02 (m, 0.8H), 3.75-3.68 (m, 3H), 3.59 (s, 1.2H), 2.80 (s, 3H). MS (EI) for C₃₁H₂₆FN₃O₅S: 572 (M+H).

4-[(3-Fluoro-2-methyl-4-{[4-(methyloxy)phenyl]-sulfonyl}phenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.94-7.72 (m, 6H), 7.61-7.54 (m, 1H), 7.40 (dd, 0.5H), 7.26 (d, 0.5H), 7.18-7.06 (m, 3.5H), 6.78 (d, 0.5H), 4.97-4.83 (m, 1H), 4.49-3.98 (m, 4H), 3.84-3.79 (m, 3H), 3.56-3.50 (m, 1H), 2.84-2.79 (m, 3H), 1.99 (d, 1.7H), 1.73 (d, 1.3H). MS (EI) for C₃₂H₂₈FN₃O₅S: 586 (M+H).

7-(2-Methyl-1H-benzimidazol-6-yl)-4-{[4-(2,2,2-trifluoroethyl)-phenyl]carbonyl}-2,3,4,5-tetrahydro-1,4-benzoxazepine. ¹H NMR (400 MHz, DMSO-d₆): δ 7.96 (s, 0.5H), 7.87-7.72 (m, 2.5H), 7.64-7.25 (m, 5.5H), 7.18-7.07 (m, 1H), 6.85 (s, 0.5H), 4.88 (s, 1.3H), 4.54 (s, 0.7H), 4.34-3.97 (m, 3H), 3.85-3.66 (m, 3H), 2.82 (s, 3H). MS (EI) for C₂₆H₂₂F₃N₃O₂: 466 (M+H).

7-(2-Methyl-1H-benzimidazol-6-yl)-4-{[2-methyl-4-(2,2,2-trifluoroethyl)phenyl]carbonyl}-2,3,4,5-tetrahydro-1,4-benzoxazepine. ¹H NMR (400 MHz, DMSO-d₆): δ 7.97 (s, 0.5H), 7.86-7.72 (m, 2.5H), 7.64-7.55 (m, 1H), 7.45 (dd, 0.5H), 7.32-7.20 (m, 2H), 7.18-7.08 (m, 2H), 6.55 (d, 0.5H), 4.99-4.81 (m, 1H), 4.45-3.95 (m, 4H), 3.80-3.52 (m, 4H), 2.81 (s, 3H), 2.14 (s, 1.5H), 1.89 (s, 1.5H). MS (EI) for C₂₇H₂₄F₃N₃O₂: 480 (M+H).

7-(2-Methyl-1H-benzimidazol-6-yl)-4-(1,2,3-thiadiazol-4-ylcarbonyl)-2,3,4,5-tetrahydro-1,4-benzoxazepine. ¹H NMR (400 MHz, DMSO-d₆): δ 12.2-12.0 (bs, 1H), 9.61-9.52 (d, 1H), 7.69-7.41 (m, 4H), 7.23-7.07 (m, 2H), 4.93 (d, 2H), 4.28-4.01 (m, 4H), 1.91 (s, 3H); MS (EI) for C₂₀H₁₇N₅O₂S: 392.1 (MH+).

4-[(2,4-Dimethyl-1,3-thiazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 11.9 (bs, 1H), 7.73-7.20 (m, 5H), 7.02 (d, 1H), 4.71 (bs, 2H), 4.15 (bs, 2H), 3.88 (bs, 2H), 2.61 (s, 3H), 2.51 (s, 3H), 2.48 (s, 3H); MS (EI) for C₂₃H₂₂N₄O₂S: 419.13 (MH⁺).

7-(2-Methyl-1H-benzimidazol-6-yl)-4-[(4-methyl-1,2,3-thiadiazol-5-yl)carbonyl]-2,3,4,5-tetrahydro-1,4-benzoxazepine. ¹H NMR (400 MHz, DMSO-d₆): δ 12.3 (bs, 1H), 7.72-7.36 (m, 4H), 7.26-6.94 (m, 2H), 4.90 (s, 1H), 4.53 (s, 1H), 4.29 (s, 1H), 4.14 (d, 2H), 3.67 (s, 1H), 2.59 (s, 3H), 2.92 (s, 3H); MS (EI) for C₂₁H₂₀N₅O₂S: 406.11 (MH⁺).

7-(2-Methyl-1H-benzimidazol-6-yl)-4-[(4-methyl-2-pyrimidin-2-yl-1,3-thiazol-5-yl)carbonyl]-2,3,4,5-tetrahydro-1,4-benzoxazepine. ¹H NMR (400 MHz, DMSO-d₆): δ 12.2 (bs, 1H), 9.35 (s, 1H), 8.80-8.69 (m, 2H), 7.75-7.04 (m, 6H), 4.74 (m, 2H), 4.22 (bs, 2H), 3.98 (m, 2H), 2.39 (s, 3H), 2.21 (s, 3H); MS (EI) for C₂₆H₂₂N₆O₂S: 483.11 (MH⁺).

7-(2-Methyl-1H-benzimidazol-5-yl)-4-[(4-methyl-1,3-thiazol-5-yl)carbonyl]-2,3,4,5-tetrahydro-1,4-benzoxazepine. ¹H NMR (400 MHz, DMSO-d₆): δ 11.9 (bs, 1H), 9.12 (s, 1H), 7.52-7.04 (m, 6H), 4.71 (m, 2H), 4.19 (m, 2H), 3.92 (m, 2H), 2.33 (s, 3H), 2.10 (s, 3H); MS (EI) for C₂₂H₂₀N₄O₂S: 405.19 (MH⁺).

1,1-Dimethylethyl (5-{[7-(2-methyl-1H-benzimidazol-5-yl)-2,3-dihydro-1,4-benzoxazepin-4(5H)-yl]carbonyl}-1,3-thiazol-2-yl)carbamate. ¹H NMR (400 MHz, DMSO-d₆): δ 11.7 (s, 1H), 7.78-7.63 (m, 6H), 7.04 (d, 1H), 4.90 (s, 2H), 4.33 (s, 2H), 4.08 (bs, 2H), 2.64 (s, 3H), 1.47 (s, 9H); MS (EI) for C₂₆H₂₇N₅O₄S: 505.9 (MH⁺).

1,1-Dimethylethyl 4-(4-methyl-5-{[7-(2-methyl-1H-benzimidazol-5-yl)-2,3-dihydro-1,4-benzoxazepin-4(5H)-yl]carbonyl}-1,3-thiazol-2-yl)piperidine-1-carboxylate. ¹H NMR (400 MHz, DMSO-d₆): δ 12.9 (bs, 1H), 7.85-7.25 (m, 5H), 7.05 (d, 1H), 4.69 (bs, 2H), 4.18 (s, 2H), 3.94 (s, 4H), 3.15 (m, 1H), 2.83 (m, 2H), 2.54 (s, 3H), 2.17 (m, 2H), 1.94 (s, 3H), 1.51 (m, 2H), 1.39 (s, 9H); MS (EI) for C₃₂H₃₇N₅O₄S: 588.01 (MH⁺).

7-(2-Methyl-1H-benzimidazol-5-yl)-4-(1,3-thiazol-5-ylcarbonyl)-2,3,4,5-tetrahydro-1,4-benzoxazepine. ¹H NMR (400 MHz, DMSO-d₆): δ 11.9 (bs, 1H), 9.25 (s, 1H), 8.18 (m, 1H), 7.79-7.35 (m, 5H), 7.05 (m, 1H), 4.88 (s, 2H), 4.30 (bs, 2H), 4.06 (bs, 2H), 2.58 (s, 3H); MS (EI) for C₂ᵢH₁₈N₄O₂S: 391.1 (MH⁺).

N-(4-Methyl-5-{[7-(2-methyl-1H-benzimidazol-5-yl)-2,3-dihydro-1,4-benzoxazepin-4(5H)-yl]carbonyl}-1,3-thiazol-2-yl)methanesulfonamide. ¹H NMR (400 MHz, DMSO-d₆): δ 12.7 (bs, 1H), 7.92 (s, 1H), 7.63 (m, 2H), 7.08 (d, 1H), 4.78 (s, 2H), 4.22 (s, 2H), 3.94 (s, 2H), 2.87 (s, 3H), 2.79 (s, 3H), 2.09 (s, 3H); MS (EI) for C₂₃H₂₃N₅O₄S₂: 497.9 (MH⁺).

7-(2-Methyl-1H-benzimidazol-5-yl)-4-(1,3-thiazol-2-ylcarbonyl)-2,3,4,5-tetrahydro-1,4-benzoxazepine. ¹H NMR (400 MHz, DMSO-d₆): δ 12.2 (bs, 1H), 8.16-8.05 (m, 1H), 8.03 (m, 1H), 7.64 (m, 1H), 7.50 (m, 3H), 7.39-7.29 (m, 1H), 7.06 (m, 1H), 5.49 (s, 1H), 4.86 (s, 1H), 4.68 (m, 1H), 4.24 (m, 2H), 4.09 (m, 1H); MS (EI) for C₂ᵢH₁₈N₄O₂S: 391.1 (MH⁺).

7-(2-Methyl-1H-benzimidazol-5-yl)-4-[(4-methyl-4H-furo[3,2-b]pyrrol-5-yl)carbonyl]-2,3,4,5-tetrahydro-1,4-benzoxazepine. ¹H NMR (400 MHz, DMSO-d₆): δ 12.2 (bs, 1H), 7.66 (m, 1H), 7.59-7.31 (m, 4H), 7.30 (m, 1H), 7.05 (d, 1H), 6.75 (s, 1H), 6.32 (s, 1H), 4.84 (s, 2H), 4.26 (s, 2H), 4.04 (m, 2H), 3.62 (s, 3H), 2.50 (s, 3H); MS (EI) for C₂₅H₂₂N₄O₃: 427.1 (MH⁺).

7-(2-Methyl-1H-benzimidazol-5-yl)-4-[(1,2,5-trimethyl-1H-pyrrol-3-yl)carbonyl]-2,3,4,5-tetrahydro-1,4-benzoxazepine. ¹H NMR (400 MHz, DMSO-d₆): δ 12.2 (s, 1H), 7.42-7.35 (m, 5H), 7.02 (d, 1H), 5.75 (s, 1H), 4.70 (s, 2H), 4.15 (bs, 2H), 3.92 (bs, 2H), 3.34 (s, 3H), 2.74 (s, 3H), 2.11 (d, 6H); MS (EI) for C₂₅H₂₆N₄O₂: 415.2 (MH⁺).

4-{[1-(4-Chlorophenyl)-3-(trifluoromethyl)-1H-pyrazol-4-yl]carbonyl}-7-(2-methyl-1H-benzimidazol-5-yl)-2,3,4,5-tetrahydro-1,4-benzoxazepine. ¹H NMR (400 MHz, DMSO-d₆): δ 12.3 (bs, 1H), 8.01 (d, 1H), 7.69-7.59 (m, 3H), 7.54-7.18 (m, 5H), 7.06-6.93 (m, 1H), 4.86 (s, 1H), 4.64 (s, 1H), 4.16 (s, 2H), 4.06 (s, 1H), 3.84 (s, 1H), 2.52 (s, 3H); MS (EI) for C₂₈H₂₁ClF₃N₅O₂: 552.2 (MH⁺).

7-(2-Methyl-1H-benzimidazol-5-yl)-4-{[4-(1H-1,2,4-triazol-1-yl)phenyl]carbonyl}-2,3,4,5-tetrahydro-1,4-benzoxazepine. ¹H NMR (400 MHz, DMSO-d₆): δ 11.9 (s, 1H), 9.41 (m, 1H), 8.28 (s, 1H), 7.95 (m, 2H), 7.71-7.40 (m, 6H), 7.25-6.94 (m, 2H), 4.86 (s, 1H), 4.61 (s, 1H), 4.29 (s, 1H), 4.16 (s, 1H), 4.03 (s, 1H), 3.80 (s, 1H), 2.50 (s, 3H); MS (EI) for C₂₆H₂₂N₆O₂: 451.2 (MH⁺).

4-{[1-(1,1-Dimethylethyl)-3-methyl-1H-pyrazol-5-yl]carbonyl}-7-(2-methyl-1H-benzimidazol-5-yl)-2,3,4,5-tetrahydro-1,4-benzoxazepine. ¹H NMR (400 MHz, DMSO-d₆): δ 12.2 (s, 1H), 7.76-7.23 (m, 4H), 7.04 (dd, 2H), 6.96 (s, 1H), 4.85 (s, 1H), 4.62 (s, 1H), 4.20 (dd, 2H), 4.02 (s, 1H), 3.72 (s, 1H), 2.50 (s, 3H), 2.15 (d, 3H), 1.40 (d, 9H); MS (EI) for C₂₆H₂₉N₅O₂: 444.2 (MH⁺).

1-(1-Methyl-5-{[7-(2-methyl-1H-benzimidazol-5-yl)-2,3-dihydro-1,4-benzoxazepin-4(5H)-yl]carbonyl}-1H-pyrrol-3-yl)ethanone. ¹H NMR (400 MHz, DMSO-d₆): δ 11.9 (s, 1H), 7.71 (s, 1H), 7.62 (s, 1H), 7.48 (m, 2H), 7.37 (d, 1H), 7.03 (d, 1H), 6.62 (bs, 1H), 4.79 (s, 2H), 4.24 (bs, 2H), 3.99 (bs, 2H), 3.59 (s, 3H), 2.50 (s, 3H), 2.42 (s, 3H); MS (EI) for C₂₅H₂₄N₄O₃: 429.2 (MH⁺).

7-(2-Methyl-1H-benzimidazol-5-yl)-4-{[2-methyl-4-(trifluoromethyl)-1,3-thiazol-5-yl]carbonyl}-2,3,4,5-tetrahydro-1,4-benzoxazepine. ¹H NMR (400 MHz, DMSO-d₆): δ 12.6 (s, 1H), 7.64-7.38 (m, 4H), 7.26-7.13 (m, 1H), 7.05 (m, 1H), 4.86 (s, 1H), 4.62 (s, 1H), 4.32 (m, 1H), 4.11 (m, 1H), 4.04 (m, 1H), 3.72 (m, 1H), 2.77-2.72 (d, 3H), 2.49 (s, 3H); MS (EI) for C₂₃H₁₉F₃N₄O₂S: 473.1 (MH⁺).

7-(2-Methyl-1H-benzimidazol-5-yl)-4-{[3-(2-methyl-1,3-thiazol-4-yl)phenyl]carbonyl}-2,3,4,5-tetrahydro-1,4-benzoxazepine. ¹H NMR (400 MHz, DMSO-d₆): δ 12.6 (s, 1H), 8.02-7.93 (m, 2H), 7.75 (m, 2H), 7.53-7.27 (m, 5H), 7.07-6.85 (m, 2H), 4.87 (s, 1H), 4.54 (s, 1H), 4.29 (s, 1H), 4.14 (s, 1H), 4.04 (s, 1H), 3.80 (s, 1H), 2.51 (d, 3H), 2.50 (s, 3H); MS (EI) for C₂₈H₂₄N₄O₂S: 481.2 (MH⁺).

7-(2-Methyl-1H-benzimidazol-5-yl)-4-[(5-methyl-1-phenyl-1H-pyrazol-4-yl)carbonyl]-2,3,4,5-tetrahydro-1,4-benzoxazepine. ¹H NMR (400 MHz, DMSO-d₆): δ 12.5 (s, 1H), 7.85-7.04 (m, 9H), 7.07 (m, 1H), 4.80 (s, 2H), 4.25 (s, 2H), 4.00 (s, 2H), 2.50 (s, 3H), 2.29-2.02 (d, 3H); MS (EI) for C₂₈H₂₅N₅O₂: 464.2 (MH⁺).

4-{[2,5-Dimethyl-1-(2-thienylmethyl)-1H-pyrrol-3-yl]carbonyl}-7-(2-methyl-1H-benzimidazol-5-yl)-2,3,4,5-tetrahydro-1,4-benzoxazepine. ¹H NMR (400 MHz, DMSO-d₆): δ 12.5 (bs, 1H), 7.75-7.24 (m, 6H), 7.04-6.88 (m, 3H), 5.81 (s, 1H), 5.21 (s, 2H), 4.72 (s, 2H), 4.17 (s, 2H), 3.92 (s, 2H), 2.50 (s, 3H), 2.18 (d, 6H); MS (EI) for C₂₉H₂₈N₄O₂S: 497.1 (MH⁺).

4-[(1-Ethyl-1H-indol-2-yl)carbonyl]-7-(2-methyl-1H-benzimidazol-5-yl)-2,3,4,5-tetrahydro-1,4-benzoxazepine. ¹H NMR (400 MHz, DMSO-d₆): δ 12.2 (bs, 1H), 7.79-7.34 (m, 6H), 7.31-6.94 (m, 4H), 6.61 (m, 1H), 4.90 (m, 2H), 4.22 (d, 4H), 4.05 (s, 2H), 2.50 (s, 3H), 1.13 (m, 3H); MS (EI) for C₂₈H₂₆N₄O₂: 451.2 (MH⁺).

7-(2-Methyl-1H-benzimidazol-5-yl)-4-[(4-methyl-4H-thieno[3,2-b]pyrrol-5-yl)carbonyl]-2,3,4,5-tetrahydro-1,4-benzoxazepine. ¹H NMR (400 MHz, DMSO-d₆): δ 12.1 (bs, 1H), 7.62 (bs, 1H), 7.53-7.45 (m, 3H), 7.43 (d, 1H), 7.31 (bs, 1H), 7.18 (d, 1H), 7.06 (d, 1H), 6.62 (bs, 1H), 4.85 (s, 2H), 4.25 (s, 2H), 4.07 (s, 2H), 3.69 (s, 3H), 2.50 (s, 3H); MS (EI) for C₂₅H₂₂N₄O₂S: 443.0 (MH⁺).

4-(1H-Indol-2-ylcarbonyl)-7-(2-methyl-1H-benzimidazol-5-yl)-2,3,4,5-tetrahydro-1,4-benzoxazepine. ¹H NMR (400 MHz, DMSO-d₆): δ 12.2 (bs, 1H), 11.6 (s, 1H), 7.74-7.31 (m, 7H), 7.18 (m, 1H), 7.05 (m, 2H), 6.83 (m, 1H), 4.94 (bs, 2H), 4.36-4.07 (m, 4H), 2.50 (s, 3H); MS (EI) for C₂₆H₂₂N₄O₂: 423.1 (MH⁺).

4-[(5-Fluoro-1-methyl-1H-indol-2-yl)carbonyl]-7-(2-methyl-1H-benzimidazol-5-yl)-2,3,4,5-tetrahydro-1,4-benzoxazepine. ¹H NMR (400 MHz, DMSO-d₆): δ 12.2 (bs, 1H), 7.67 (s, 1H), 7.55 (m, 3H), 7.42 (m, 2H), 7.10 (m, 3H), 6.70-6.52 (m, 1H), 4.89 (m, 2H), 4.25 (m, 2H), 4.02 (s, 2H), 3.66 (s, 3H), 2.50 (s, 3H); MS (EI) for C₂₇H₂₃FN₄O₂: 455.2 (MH⁺).

4-[(5-Chloro-1-methyl-1H-indol-2-yl)carbonyl]-7-(2-methyl-1H-benzimidazol-5-yl)-2,3,4,5-tetrahydro-1,4-benzoxazepine. ¹H NMR (400 MHz, DMSO-d₆): δ 12.2 (bs, 1H), 7.76-7.31 (m, 6H), 7.26 (d, 1H), 7.07 (d, 1H), 7.01-6.53 (m, 2H), 4.95-4.67 (m, 2H), 4.26 (m, 2H), 4.03 (m, 2H), 3.68-3.49 (d, 3H), 2.50 (s, 3H); MS (EI) for C₂₇H₂₃ClN₄O₂: 471.2 (MH⁺).

4-[(1-Acetyl-1H-indol-2-yl)carbonyl]-7-(2-methyl-1H-benzimidazol-5-yl)-2,3,4,5-tetrahydro-1,4-benzoxazepine. ¹H NMR (400 MHz, DMSO-d₆): δ 12.1 (bs, 1H), 8.13 (m, 1H), 7.74-7.24 (m, 7H), 7.21-6.66 (m, 3H), 4.89 (s, 1H), 4.76 (s, 1H), 4.26 (d, 2H), 4.03 (d, 2H), 2.50 (s, 3H), 2.41 (s, 3H); MS (EI) for C₂₈H₂₄N₄O₂: 465.2 (MH⁺).

4-[(4-Ethyl-4H-furo[3,2-b]pyrrol-5-yl)carbonyl]-7-(2-methyl-1H-benzimidazol-5-yl)-2,3,4,5-tetrahydro-1,4-benzoxazepine. ¹H NMR (400 MHz, DMSO-d₆): δ 12.1 (bs, 1H), 7.67 (dd, 1H), 7.60 (bs, 1H), 7.48 (m, 3H), 7.31 (m, 1H), 7.04 (d, 1H), 6.77 (m, 1H), 6.33 (bs, 1H), 4.86 (s, 2H), 4.25 (s, 2H), 4.03 (m, 4H), 2.50 (s, 3H), 1.14 (m, 3H); MS (EI) for C₂₆H₂₄N₄O₃: 441.2 (MH⁺).

7-(2-Methyl-1H-benzimidazol-5-yl)-4-[(6-methyl-6H-thieno[2,3-b]pyrrol-5-yl)carbonyl]-2,3,4,5-tetrahydro-1,4-benzoxazepine. ¹H NMR (400 MHz, DMSO-d₆): δ 12.0 (bs, 1H), 7.65 (bs, 1H), 7.51 (dd, 3H), 7.30 (bs, 1H), 7.12 (d, 1H), 7.04 (dd, 2H), 6.58 (s, 1H), 4.86 (s, 2H), 4.27 (s, 2H), 4.08 (s, 2H), 3.65 (s, 3H), 2.50 (s, 3H); MS (EI) for C₂₅H₂₂N₄O₂S: 443.2 (MH⁺).

4-[(3-Chlorophenyl)carbonyl]-7-(2-methyl-1H-benzimidazol-5-yl)-2,3,4,5-tetrahydro-1,4-benzoxazepine. ¹H NMR (400 MHz, DMSO-d₆): δ 12.2 (bs, 1H), 7.66 (s, 1H), 7.62-7.43 (m, 5H), 7.42-7.33 (m, 1H), 7.30-6.93 (m, 3H), 4.84 (s, 1H), 4.52 (s, 1H), 4.28 (s, 1H), 4.14 (s, 1H), 4.01 (s, 1H), 3.74 (s, 1H), 2.50 (s, 3H); MS (EI) for C₂₄H₂₀ClN₃O₂: 418.3 (MH⁺).

7-(2-Methyl-1H-benzimidazol-5-yl)-4-[(1-propyl-1H-indol-2-yl)carbonyl]-2,3,4,5-tetrahydro-1,4-benzoxazepine. ¹H NMR (400 MHz, DMSO-d₆): δ 12.0 (bs, 1H), 7.73-7.36 (m, 6H), 7.23 (t, 1H), 7.08 (m, 3H), 6.61 (m, 1H), 4.90 (s, 2H), 4.27 (s, 2H), 4.13 (s, 2H), 4.05 (s, 2H), 2.50 (s, 3H), 1.51 (t, 3H), 0.65 (t, 3H); MS (EI) for C₂₉H₂₈N₄O₂: 465.3 (MH⁺).

7-(2-Methyl-1H-benzimidazol-5-yl)-4-{[1-(1-methylethyl)-1H-indol-2-yl]carbonyl}-2,3,4,5-tetrahydro-1,4-benzoxazepine. ¹H NMR (400 MHz, DMSO-d₆): δ 12.2 (d, 1H), 7.75-7.32 (m, 6H), 7.20 (m, 1H), 7.08 (m, 2H), 6.98 (m, 1H), 6.57-6.39 (d, 1H), 4.91 (s, 1H), 4.81 (s, 1H), 4.61 (m, 1H), 4.29 (s, 1H), 4.19 (s, 1H), 4.08 (s, 1H), 3.95 (s, 1H), 2.48 (s, 3H), 1.47 (d, 3H), 1.40 (d, 3H); MS (EI) for C₂₉H₂₈N₄O₂: 465.3 (MH⁺).

4-[(4-Ethyl-4H-thieno[3,2-b]pyrrol-5-yl)carbonyl]-7-(2-methyl-1H-benzimidazol-5-yl)-2,3,4,5-tetrahydro-1,4-benzoxazepine. ¹H NMR (400 MHz, DMSO-d₆): δ 12.2 (s, 1H), 7.60 (bs, 1H), 7.52-7.39 (m, 4H), 7.29 (bs, 1H), 7.19 (dd, 1H), 7.05 (dd, 1H), 6.64 (s, 1H), 4.87 (s, 2H), 4.25 (s, 2H), 4.15 (m, 2H), 4.06 (s, 2H), 2.50 (s, 3H), 1.12 (m, 2H); MS (EI) for C₂₆H₂₄N₄O₂S: 457.12 (MH⁺).

N-(1,1-Dimethylethyl)-1-methyl-5-{[7-(2-methyl-1H-benzimidazol-6-yl)-2,3-dihydro-1,4-benzoxazepin-4(5H)-yl]carbonyl}-1H-pyrrole-2-sulfonamide. ¹H NMR (400 MHz, DMSO-d₆): δ 12.2 (bs, 1H), 7.74-7.31 (m, 5H), 7.07 (d, 2H), 6.52 (s, 1H), 4.80 (s, 2H), 4.25 (s, 2H), 3.99 (s, 2H), 3.56 (s, 3H), 1.10 (s, 9H); MS (EI) for C₂₇H₃₁N₅O₄S: 522.0 (MH⁺).

7-(2-Methyl-1H-benzimidazol-5-yl)-4-[(2-methyl-4H-thieno[3,2-b]pyrrol-5-yl)carbonyl]-2,3,4,5-tetrahydro-1,4-benzoxazepine. ¹H NMR (400 MHz, DMSO-d₆): δ 12.3 (bs, 1H), 11.5 (bs, 1H), 7.72-7.32 (m, 5H), 7.00 (d, 1H), 6.78 (s, 1H), 6.69 (s, 1H), 4.92 (s, 2H), 4.31 (s, 2H), 4.15 (s, 2H), 2.47 (s, 3H), 1.81 (s, 3H); MS (EI) for C₂₅H₂₂N₄O₂S: 443.0 (MH⁺).

4-[(2,4-Dimethyl-4H-pyrrolo[3,2-d][1,3]thiazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.3 (bs, 1H), 7.67-7.28 (m, 5H), 7.06 (d, 1H), 6.61 (bs, 1H), 4.86 (s, 2H), 4.28 (s, 2H), 4.03 (m, 2H), 3.67 (s, 3H), 2.69 (s, 3H); MS (EI) for C₂₅H₂₃N₅O₂S: 458.2 (MH⁺).

7-(2-Methyl-1H-benzimidazol-6-yl)-4-{[1-methyl-5-(methylsulfonyl)-1H-indol-2-yl]carbonyl}-2,3,4,5-tetrahydro-1,4-benzoxazepine. ¹H NMR (400 MHz, DMSO-d₆): δ 12.3 (bs, 1H), 8.23 (m, 1H), 7.66-7.44 (m, 6H), 7.10-6.94 (m, 3H), 4.92 (s, 1H), 4.74 (s, 1H), 4.30 (d, 2H), 4.21 (d, 2H), 3.76-3.55 (d, 3H), 3.26-3.18 (d, 3H); MS (EI) for C₂₈H₂₆N₄O₄S: 515.2 (MH⁺).

7-(2-Methyl-1H-benzimidazol-6-yl)-4-[(3,4,6-trichloro-1-benzothien-2-yl)carbonyl]-2,3,4,5-tetrahydro-1,4-benzoxazepine. ¹H NMR (400 MHz, DMSO-d₆): δ 12.3 (bs, 1H), 8.35 (d, 1H), 7.80-7.01 (m, 7H), 4.90 (s, 1H), 4.66 (s, 1H), 4.25-4.09 (m, 3H), 3.83 (s, 1H); MS (EI) for C₂₆H₁₈Cl₃N₃O₂S: 542.0 (MH⁺).

4-[(3-Chloro-4-fluoro-1-benzothien-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.2 (bs, 1H), 7.96 (m, 1H), 7.69 (s, 1H), 7.55-7.54 (m, 5H), 7.07 (m, 1H), 4.90 (s, 1H), 4.69 (s, 1H), 4.25-4.16 (m, 3H), 3.86 (s, 1H); MS (EI) for C₂₆H₁₉ClFN₃O₂S: 492.1, (M⁺).

4-[(3-Chloro-6-fluoro-1-benzothien-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.2 (bs, 1H), 8.10 (s, 1H), 7.88 (m, 1H), 7.75-7.32 (m, 6H), 7.07 (d, 1H), 4.90 (s, 1H), 4.69 (s, 1H), 4.28-4.01 (m, 3H), 3.87 (s, 1H); MS (EI) for C₂₆H₁₉ClFN₃O₂S: 492.1 (M⁺).

7-(2-Methyl-1H-benzimidazol-6-yl)-4-(thieno[2,3-b]pyridin-2-ylcarbonyl)-2,3,4,5-tetrahydro-1,4-benzoxazepine. ¹H NMR (400 MHz, DMSO-d₆): δ 12.1 (bs, 1H), 8.66 (d, 1H), 7.76-7.15 (m, 7H), 7.07 (d, 1H), 4.92 (s, 2H), 4.31 (s, 2H), 4.11 (m, 2H), 2.50 (s, 3H); MS (EI) for C₂₅H₂₀N₄O₂S: 441.2 (MH⁺).

7-(2-Methyl-1H-benzimidazol-6-yl)-4-(thieno[2,3-b]pyrazin-6-ylcarbonyl)-2,3,4,5-tetrahydro-1,4-benzoxazepine. ¹H NMR (400 MHz, DMSO-d₆): δ 12.3 (bs, 1H), 8.85 (s, 1H), 8.71 (s, 1H), 7.98-7.08 (m, 7H), 4.93 (s, 2H), 4.33 (d, 2H), 4.12 (s, 2H), 2.49 (s, 3H); MS (EI) for C₂₄H₁₉N₅O₂S: 442.2 (MH⁺).

4-[(4-Ethyl-2-methyl-4H-pyrrolo[3,2-d][1,3]thiazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 7.69-7.15 (m, 5H), 7.05 (d, 1H), 6.65 (s, 1H), 4.88 (s, 2H), 4.27 (s, 2H), 4.06 (m, 4H), 2.69 (s, 3H), 1.89 (s, 3H); MS (EI) for C₂₆H₂₅N₅O₂S: 471.9 (MH⁺).

7-(2-Methyl-1H-benzimidazol-6-yl)-4-[(1-methyl-1H-pyrrol-2-yl)carbonyl]-2,3,4,5-tetrahydro-1,4-benzoxazepine. ¹H NMR (400 MHz, DMSO-d₆): δ 7.66-7.32 (m, 5H), 7.04 (d, 1H), 6.90 (s, 1H), 6.27 (s, 1H), 6.04 (s, 1H), 4.80 (s, 2H), 4.24 (s, 2H), 4.01 (s, 2H), 3.56 (s, 3H); MS (EI) for C₂₃H₂₂N₄O₂: 387.1 (MH⁺).

7-(2-Methyl-1H-benzimidazol-6-yl)-4-[(1-methyl-1H-pyrazol-5-yl)carbonyl]-2,3,4,5-tetrahydro-1,4-benzoxazepine. ¹H NMR (400 MHz, DMSO-d₆): δ 12.2 (bs, 1H), 7.71-7.21 (m, 6H), 7.01 (d, 1H), 6.43 (d, 1H), 4.86 (s, 1H), 4.68 (s, 1H), 4.24 (d, 2H), 4.02 (s, 1H), 3.90 (s, 1H), 3.75 (s, 3H), 3.55 (s, 3H); MS (EI) for C₂₂H₂ᵢN₅O₂: 388.1 (MH⁺).

4-[(2,4-Dimethyl-4H-thieno[3,2-b]pyrrol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.2 (bs, 1H), 7.66-7.29 (m, 5H), 7.06 (d, 1H), 6.90 (s, 1H), 6.54 (s, 1H), 4.84 (s, 2H), 4.25 (s, 2H), 4.06 (s, 2H), 3.63 (s, 3H); MS (EI) for C₂₆H₂₄N₄O₂S: 456.9 (MH⁺).

4-[(4-Ethyl-2-methyl-4H-thieno[3,2-b]pyrrol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.2 (bs, 1H), 7.64-7.21 (m, 5H), 7.03 (d, 1H), 6.91 (s, 1H), 6.56 (s, 1H), 4.86 (s, 2H), 4.24 (m, 2H), 4.05 (m, 4H), 1.09 (m, 3H); MS (EI) for C₂₇H₂₆N₄O₂S: 471.1 (MH⁺).

7-(2-Methyl-1H-benzimidazol-6-yl)-4-[(1-methyl-1H-imidazol-5-yl)carbonyl]-2,3,4,5-tetrahydro-1,4-benzoxazepine. ¹H NMR (400 MHz, DMSO-d₆): δ 12.2 (bs, 1H), 7.76 (s, 1H), 7.71-7.45 (m, 5H), 7.36 (m, 1H), 7.05 (d, 1H), 4.84 (s, 2H), 4.27 (s, 2H), 4.03 (s, 2H), 3.61 (s, 3H); MS (EI) for C₂₂H₂ᵢN₅O₂: 388.1 (MH⁺).

4-[(1-Ethyl-1H-imidazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.00-7.02 (m, 8H), 4.86 (s, 2H), 4.26 (s, 2H), 4.03 (m, 4H), 2.58 (s, 3H), 1.07 (bs, 3H); MS (EI) for C₂₃H₂₃N₅O₂: 402.0 (MH⁺).

4-[(4-Chloro-1-ethyl-1H-pyrazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 7.76-7.66 (m, 2H), 7.55-7.39 (m, 3H), 7.23-6.75 (m, 2H), 4.91 (m, 1H), 4.71-4.49 (dd, 1H), 4.42-4.31 (m, 1H), 4.29-3.88 (m, 2H), 3.77 (m, 2H), 3.64 (q, 2H), 2.49 (s, 3H), 1.21-0.95 (m, 3H); MS (EI) for C₂₃H₂₂ClN₅O₂: 435.9 (M⁺).

4-[(1-Ethyl-1H-pyrrol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.2 (s, 1H), 11.9 (bs, 1H), 7.69-7.31 (m, 5H), 7.04 (d, 1H), 6.96 (s, 1H), 6.30 (m, 1H), 6.03 (s, 1H), 4.82 (s, 2H), 4.22 (s, 2H), 3.97 (m, 4H), 1.90 (s, 3H), 1.04 (s, 3H); MS (EI) for C₂₄H₂₄N₄O₂: 401.0 (MH⁺).

4-[(1-Ethyl-1H-pyrazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.2 (s, 1H), 7.72-7.35 (m, 5H), 7.24-7.17 (m, 1H), 7.07-6.92 (m, 2H), 6.48-6.40 (d, 1H), 4.86 (s, 1H), 4.72 (s, 1H), 4.24 (m, 2H), 4.10 (m, 1H), 4.03 (m, 1H), 3.91 (m, 2H), 1.27-0.39 (m, 3H); MS (EI) for C₂₃H₂₃N₅O₂: 402.1 (MH⁺).

7-(2-Methyl-1H-benzimidazol-6-yl)-4-({4-[3-(trifluoromethyl)-1H-pyrazol-1-yl]phenyl}carbonyl)-2,3,4,5-tetrahydro-1,4-benzoxazepine. ¹H NMR (400 MHz, DMSO-d₆): δ 12.2 (d, 1H), 7.96-6.83 (m, 12H), 4.89 (s, 1H), 4.56 (s, 1H), 4.29-4.20 (d, 2H), 4.04 (s, 1H), 3.79 (s, 1H), 2.50 (s, 3H); MS (EI) for C₂₈H₂₂F₃N₅O₂: 517.9 (M⁺).

4-{[4-(1H-Benzimidazol-1-ylmethyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 12.3 (bs, 1H), 8.47 (d, 1H), 7.71-6.98 (m, 13H), 6.77 (s, 1H), 5.55 (d, 2H), 4.81 (s, 1H), 4.48 (s, 1H), 4.25 (s, 1H), 4.09 (s, 1H), 3.97 (s, 1H), 3.70 (s, 1H); MS (EI) for C₃₂H₂₇N₅O₂: 514.0 (MH⁺).

7-(2-Methyl-1H-benzimidazol-6-yl)-4-{[5-(2-methyl-1,3-thiazol-4-yl)isoxazol-3-yl]carbonyl}-2,3,4,5-tetrahydro-1,4-benzoxazepine. ¹H NMR (400 MHz, DMSO-d₆): δ 12.2 (bs, 1H), 8.30 (d, 1H), 7.77-7.38 (m, 4H), 7.29 (m, 1H), 7.07 (m, 2H), 4.86 (d, 2H), 4.22 (dd, 2H), 4.08 (dd, 2H), 2.72 (d, 3H); MS (EI) for C₂₅H₂₁N₅O₃S: 471.9 (MH⁺).

N-tert-Butyl-3-chloro-4-{[7-(2-methyl-1H-benzimidazol-6-yl)-2,3-dihydro-1,4-benzoxazepin-4(5H)-yl]carbonyl}benzensulfonamide. ¹H NMR (400 MHz, DMSO-d₆): δ 12.3 (br, 1H), 7.54 (m, 9H), 4.85 (dd, 1H), 4.50 (dd, 1H), 4.20 (m, 3H), 3.65 (m, 1H), 2.45 (s, 3H), 1.32 (s, 9H). MS (EI) for C₂₈H₂₉ClN₄O₄S: 554.2 (MH⁺).

N-Ethyl-3-methyl-4-{[7-(2-methyl-1H-benzimidazol-6-yl)-2,3-dihydro-1,4-benzoxazepin-4(5H)-yl]carbonyl}benzenesulfonamide. ¹H NMR (400 MHz, DMSO-d₆): δ 12.2 (br, 1H), 7.98 (s, 1H), 6.50 (s, 1H), 7.10 (m, 7H), 6.50 (s, 1H), 4.98 (m, 1H), 4.48 (s, 2H), 4.25 (m, 2H), 3.52 (m, 1H), 2.75 (q, 2H), 2.47 (s, 3H), 2.30 (s, 3H), 1.51 (t, 3H). MS (EI) for C₂₇H₂₈N₄O₄S: 505.2 (MH⁺).

N-Cyclopentyl-3-methyl-4-{[7-(2-methyl-1H-benzimidazol-6-yl)-2,3-dihydro-1,4-benzoxazepin-4(5H)-yl]carbonyl}benzenesulfonamide. ¹H NMR (400 MHz, DMSO-d₆): δ 12.2 (br, 1H), 7.54 (m, 9H), 4.98 (d, 1H), 4.20 (m, 3H), 3.52 (m, 2H), 3.01 (m, 1H), 2.51 (s, 3H), 2.49 (s, 3H), 1.98 (s, 1H), 1.33 (m, 4H), 0.65 (m, 4H). MS (EI) for C₃₀H₃₂N₄O₄S: 545.2 (MH⁺).

N-Cyclopentyl-4-{[7-(2-methyl-1H-benzimidazol-6-yl)-2,3-dihydro-1,4-benzoxazepin-4(5H)-yl]carbonyl}-3-(trifluoromethyl)benzenesulfonamide. ¹H NMR (400 MHz, DMSO-d₆): δ 12.2 (br, 1H), 8.30 (m, 2H), 7.90 (s, 1H), 7.80 (m, 1H), 7.50 (m, 2H), 7.10 (m, 2H), 6.60 (s, 1H), 4.98 (d, 1H), 4.40 (m, 2H), 4.20 (m, 3H), 3.52 (m, 1H), 3.01 (m, 1H), 2.51 (s, 3H), 1.25 (m, 4H), 0.60 (m, 4H). MS (EI) for C₃₀H₂₉F₃N₄O₄S: 599.2 (MH⁺).

3-Bromo-4-[7-(2-methyl-1H-benzimidazol-6-yl)-2,3-dihydro-1,4-benzoxazepin-4(5H)-yl]carbonyl}-N-(propan-2-yl)benzenesulfonamide. ¹H NMR (400 MHz, DMSO-d₆): δ 12.2 (br, 1H), 8.01 (d, 1H), 7.98 (m, 1H), 7.60 (s, 1H), 7.50 (m, 3H), 7.20 (m, 1H), 7.10 (d, 1H), 6.50 (s, 1H), 4.98 (dd, 1H), 4.40 (dd, 1H), 4.10 (m, 4H), 3.52 (m, 1H), 2.51 (s, 3H), 0.97 (dd, 3H), 0.89 (m, 3H). MS (EI) for C₂₇H₂₇BrN₄O₄S: 584.2 (MH⁺).

N-Ethyl-4-[7-(2-methyl-1H-benzimidazol-6-yl)-2,3-dihydro-1,4-benzoxazepin-4(5H)-yl]carbonyl}-3-(trifluoromethyl)benzenesulfonamide. ¹H NMR (400 MHz, MeOH-d₄): δ 8.18 (d, 1H), 7.70 (d, 1H), 7.50 (m, 4H), 7.10 (d, 2H), 6.60 (s, 1H), 4.80 (d, 1H), 4.30 (m, 2H), 4.20 (m, 1H), 4.01 (m, 1H), 3.62 (m, 1H), 2.80 (q, 2H), 2.58 (s, 3H), 0.96 (t, 3H). MS (EI) for C₂₇H₂₅F₃N₄O₄S: 559.2 (MH⁺).

N-Isopropyl-4-[7-(2-methyl-1H-benzimidazol-6-yl)-2,3-dihydro-1,4-benzoxazepin-4(5H)-yl]carbonyl}-3-(trifluoromethyl)benzenesulfonamide. ¹H NMR (400 MHz, MeOH-d₄): δ 8.18 (d, 1H), 7.70 (d, 1H), 7.50 (m, 4H), 7.10 (d, 2H), 6.60 (s, 1H), 4.80 (d, 1H), 4.60 (d, 1H), 4.30 (m, 2H), 4.20 (m, 1H), 4.01 (m, 1H), 3.62 (m, 1H), 2.58 (s, 3H), 1.02 (d, 3H), 0.96 (d, 3H). MS (EI) for C₂₈H₂₇F₃N₄O₄S: 573.2 (MH⁺).

N-Cyclopropyl-4-[7-(2-methyl-1H-benzimidazol-6-yl)-2,3-dihydro-1,4-benzoxazepin-4(5H)-yl]carbonyl}-3-(trifluoromethyl)benzenesulfonamide. ¹H NMR (400 MHz, MeOH-d₄): δ 8.18 (d, 1H), 7.70 (d, 1H), 7.50 (m, 4H), 7.10 (d, 2H), 6.60 (s, 1H), 4.80 (d, 1H), 4.60 (d, 1H), 4.30 (m, 2H), 4.20 (m, 1H), 4.01 (m, 1H), 3.62 (m, 1H), 2.58 (s, 3H), 0.57 (m, 2H), 0.38 (m, 2H). MS (EI) for C₂₈H₂₅F₃N₄O₄S: 571.2 (MH⁺).

3-Chloro-4-{[7-(2-methyl-1H-benzimidazol-6-yl)-2,3-dihydro-1,4-benzoxazepin-4(5H)-yl]carbonyl}-N-(propan-2-yl)benzensulfonamide. ¹H NMR (400 MHz, DMSO-d₆): δ 12.10 (br, 1H), 7.70 (m, 6H), 7.02 (m, 2H), 6.52 (s, 1H), 4.81 (s, 2H), 4.35 (s, 2H), 4.02 (brs, 2H), 3.62 (m, 1H), 2.51 (s, 3H), 1.01 (d, 3H), 0.85 (d, 3H). MS (EI) for C₂₇H₂₇ClN₄O₄S: 540.2 (MH⁺).

3-Ethyl-4-{[7-(2-methyl-1H-benzimidazol-6-yl)-2,3-dihydro-1,4-benzoxazepin-4(5H)-yl]carbonyl}-N-(propan-2-yl)benzenesulfonamide. ¹H NMR (400 MHz, DMSO-d₆): δ 12.10 (br, 1H), 7.50 (m, 8H), 6.56 (s, 1H), 4.81 (dd, 1H), 4.40 (dd, 1H), 4.15 (m, 4H), 3.30 (m, 1H), 2.80 (q, 2H), 2.51 (s, 3H), 1.01 (t, 3H), 0.89 (d, 6H). MS (EI) for C₂₉H₃₂N₄O₄S: 533.1 (MH⁺).

[7-(2-Methyl-1H-benzimidazol-6-yl)-2,3-dihydro-1,4-benzoxazepin-4(5H)-yl](1-methyl-5-phenyl-1H-pyrrol-2-yl)methanone. ¹H NMR (400 MHz, DMSO-d₆): δ 12.10 (br, 1H), 7.51 (m, 10H), 7.01 (d, 1H), 6.40 (s, 1H), 6.22 (s, 1H), 4.83 (s, 3H), 4.30 (br s, 2H), 4.0 (brs, 2H), 2.50 (s, 3H). MS (EI) for C₂₉H₂₆N₄O₂: 463.1 (MH⁺).

[4-(4-Fluorophenyl)-1-methyl-1H-pyrrol-2-yl][7-(2-methyl-1H-benzimidazol-6-yl)-2,3-dihydro-1,4-benzoxazepin-4(5H)-yl]methanone. ¹H NMR (400 MHz, DMSO-d₆): δ 12.2 (br, 1H), 7.70 (s, 2H), 7.45 (m, 3H), 7.38 (m, 3H), 7.02 (d, 2H), 6.90 (br, 1H), 6.50 (br, 1H), 4.82 (s, 2H), 4.30 (s, 2H), 4.02 (s, 2H), 3.61 (s, 3H), 2.51 (s, 3H). MS (EI) for C₂₉H₂₅FN₄O₂: 481.1 (MH⁺).

N-tert-Butyl-2-iodo-4-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 12.5 (br, 1H), 8.01 (m, 2H), 7.80 (s, 1H), 7.60 (d, 1H), 7.52 (m, 2H), 7.30 (m, 2H), 7.10 (m, 1H), 4.85 (s, 1H), 4.51 (s, 1H), 4.32 (s, 1H), 4.19 (s, 1H), 4.00 (s, 1H), 3.85 (m, 1H), 3.75 (s, 1H), 2.47 (s, 3H), 1.42 (m, 9H). MS (EI) for C₂₉H₂₉IN₄O₃: 609.3 (MH+).

N-Cyclopentyl-2-iodo-4-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 12.5 (br, 1H), 8.48 (m, 1H), 7.52 (m, 9H), 4.85 (s, 1H), 4.51 (s, 1H), 4.32 (s, 1H), 4.19 (s, 1H), 4.00 (s, 1H), 3.85 (m, 1H), 3.68 (s, 1H), 2.41 (s, 3H), 1.85 (m, 2H), 1.62 (m, 2H), 1.75 (m, 4H). MS (EI) for C₃₀H₂₉IN₄O₃: 621.3 (MH⁺).

N-Ethyl-4-{[7-(2-methyl-1H-benzimidazol-6-yl)-2,3-dihydro-1,4-benzoxazepin-4(5H)-yl]carbonyl}benzenesulfonamide. ¹H NMR (400 MHz, DMSO-d₆): δ 12.46 (br s, 1H), 8.19-6.62 (m, 11H), 4.87 (s, 1H), 4.52 (s, 1H), 4.40-3.61 (m, 4H), 2.92-2.70 (m, 2H), 1.15-0.70 (m, 3H). MS (EI) for C₂₆H₂₆N₄O₄S: 491 (M+H).

N-Methyl-N-(4-{[7-(2-methyl-1H-benzimidazol-6-yl)-2,3-dihydro-1,4-benzoxazepin-4(5H)-yl]carbonyl}phenyl)methanesulfonamide. ¹H NMR (400 MHz, DMSO-d₆): δ 8.08-6.80 (m, 10H), 5.01-3.69 (m, 6H), 3.24 (s, 3H), 2.93 (s, 3H), 2.79 (s, 3H). MS (EI) for C₂₆H₂₆N₄O₄S: 491 (M+H).

4-[(1-Ethyl-3-methyl-1H-pyrazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.01-7.56 (m, 5H), 7.12 (m, 1H), 6.99-6.38 (br s, 2H), 4.98-4.72 (m, 2H), 4.38-4.18 (m, 2H), 4.10-3.76 (m, 4H), 2.82 (s, 3H), 2.26-2.01 (m, 3H), 1.34-0.93 (m, 3H). MS (EI) for C₂₄H₂₅N₅O₂: 416 (M+H).

4-{[7-(2-Methyl-1H-benzimidazol-6-yl)-2,3-dihydro-1,4-benzoxazepin-4(5H)-yl]carbonyl}-N-[2-(methylsulfonyl)ethyl]benzenesulfonamide. ¹H NMR (400 MHz, DMSO-d₆/CD₃OD): δ 8.03-7.43 (m, 8H), 7.20-7.11 (m, 1H), 6.76 (s, 1H), 4.59 (s, 1H), 4.37-4.30 (m, 1H), 4.20-4.11 (m, 2H), 3.88-3.81 (m, 1H), 3.16 (s, 2H), 3.03-2.95 (m, 3H), 2.90-2.83 (m, 3H). MS (EI) for C₂₇H₂₈N₄O₆S₂: 569 (M+H).

7-(2-Methyl-1H-benzimidazol-6-yl)-4-{[2-(methyloxy)-4-(methylsulfonyl)phenyl]carbonyl}-2,3,4,5-tetrahydro-1,4-benzoxazepine. ¹H NMR (400 MHz, DMSO-d₆): δ 7.97 (s, 0.5H), 7.87-7.72 (m, 2.5H), 7.67-7.40 (m, 4H), 7.24 (d, 0.5H), 7.16-7.10 (m, 1H), 6.73 (d, 0.5H), 4.96-4.81 (m, 1H), 4.49-3.93 (m, 4H), 3.88 (s, 1.5H), 3.63-3.54 (m, 2.5H), 3.30-3.21 (m, 3H), 2.84-2.78 (m, 3H). MS (EI) for C₂₆H₂₅N₃O₅S: 492 (M+H).

4-[(2,4-Dichlorophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.93 (s, 0.5H), 7.82-7.72 (m, 3.5H), 7.64-7.47 (m, 2.5H), 7.39 (d, 0.5H), 7.23 (d, 0.5H), 7.14-7.09 (m, 1H), 6.88 (d, 0.5H), 5.01-4.80 (m, 1H), 4.57-3.96 (m, 4H), 3.64-3.56 (m, 1H), 2.79-2.73 (m, 3H). MS (EI) for C₂₄H₁₉Cl₂N₃O₂: 452 (M+H).

4-[(4-Bromo-2-methylphenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.95 (s, 0.5H), 7.85-7.74 (m, 2.5H), 7.64-7.41 (m, 3.5H), 7.14-7.08 (m, 1.5H), 7.01 (d, 0.5H), 6.75 (d, 0.5H), 5.00-4.81 (m, 1H), 4.49-4.00 (m, 4H), 3.62-3.53 (m, 1H), 2.82-2.75 (m, 3H), 2.13 (s, 1.5H), 1.90 (s, 1.5). MS (EI) for C₂₅H₂₂BrN₃O₂: 476 (M+H).

4-[(2-Bromo-4-chlorophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.96 (s, 0.5H), 7.90-7.75 (m, 3.5H), 7.63 (dd, 0.5H), 7.59-7.50 (m, 2H), 7.35 (d, 0.5H), 7.20 (d, 0.5H), 7.15-7.09 (m, 1H), 6.75 (d, 0.5H), 5.02-4.79 (m, 1H), 4.57-3.97 (m, 4H), 3.63-3.52 (m, 1H), 2.82-2.77 (m, 3H). MS (EI) for C₂₄H₁₉BrClN₃O₂: 496 (M+H).

4-[(4-Chloro-2-fluorophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.95 (s, 0.5H), 7.85-7.75 (m, 2.5H), 7.65-7.54 (m, 2.5H), 7.46-7.35 (m, 1.5H), 7.28-7.22 (m, 0.5H), 7.12 (d, 1H), 6.99 (d, 0.5H), 4.93-4.51 (m, 2H), 4.31-4.02 (m, 2.5H), 3.72-3.66 (m, 1.5H), 2.83-2.76 (m, 3H). MS (EI) for C₂₄H₁₉ClFN₃O₂: 436 (M+H).

4-[(2-Fluoro-4-iodophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.95 (s, 0.5H), 7.85-7.75 (m, 3.5H), 7.68-7.50 (m, 2.5H), 7.19-7.09 (m, 1.5H), 7.01-6.96 (m, 0.5H), 6.92 (d, 0.5H), 4.92-4.51 (m, 2H), 4.29-4.01 (m, 2.5H), 3.71-3.65 (m, 1.5H), 2.81-2.77 (m, 3H). MS (EI) for C₂₄H₁₉FIN₃O₂: 528 (M+H).

4-[(4-Bromo-2-fluorophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.94 (s, 0.5H), 7.84-7.69 (m, 3.5H), 7.65-7.47 (m, 2.5H), 7.39-7.33 (m, 0.5H), 7.20-7.15 (m, 0.5H), 7.11 (d, 1H), 6.96 (d, 0.5H), 4.92-4.50 (m, 2H), 4.30-4.00 (m, 2.5H), 3.72-3.65 (m, 1.5H), 2.81-2.77 (m, 3H). MS (EI) for C₂₄H₁₉BrFN₃O₂: 480 (M+H).

4-[(4-Bromo-2-chlorophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.95 (s, 0.5H), 7.89-7.74 (m, 3.5H), 7.65-7.49 (m, 2.5H), 7.31 (d, 0.5H), 7.17-7.09 (m, 1.5H), 6.87 (d, 0.5H), 5.00-4.80 (m, 2H), 4.57-3.96 (m, 4H), 3.62-3.56 (m, 1H), 2.81-2.75 (m, 3H). MS (EI) for C₂₄H₁₉ClN₃O₃: 496 (M+H).

7-(2-Methyl-1H-benzimidazol-6-yl)-4-{[4-(trifluoromethyl)-phenyl]carbonyl}-2,3,4,5-tetrahydro-1,4-benzoxazepine. ¹H NMR (400 MHz, DMSO-d₆): δ 7.96 (s, 0.5H), 7.86-7.72 (m, 4.5H), 7.66-7.45 (m, 3.5H), 7.16-7.09 (m, 1H), 6.96 (s, 0.5H), 4.95-4.53 (m, 2H), 4.38-3.71 (m, 4H), 2.85-2.79 (m, 3H). MS (EI) for C₂₅H₂₀F₃N₃O₂: 452 (M+H).

7-(2-Methyl-1H-benzimidazol-6-yl)-4-{[4-(phenylsulfonyl)-phenyl]carbonyl}-2,3,4,5-tetrahydro-1,4-benzoxazepine. ¹H NMR (400 MHz, DMSO-d₆): δ 8.05-7.96 (m, 4H), 7.76-7.45 (m, 9H), 7.23 (d, 0.5H), 7.10-7.03 (m, 1H), 6.79 (s, 0.5H), 4.89-4.45 (m, 2H), 4.31-3.65 (m, 4H), 2.59 (s, 3H). MS (EI) for C₃₀H₂₅N₃O₄S: 524 (M+H).

4-[(2-Ethylphenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.94 (s, 0.5H), 7.83-7.72 (m, 2H), 7.77-7.46 (m, 2.5H), 7.36-7.18 (m, 2.5H), 7.12-7.06 (m, 1.5H), 6.99 (d, 0.5H), 6.54 (s, 0.5H), 5.04-4.77 (m, 1H), 4.47-3.51 (m, 6H), 2.83-2.77 (m, 3H), 2.47-2.10 (m, 2H), 1.06 (t, 1.5H), 0.94 (t, 1.5H). MS (EI) for C₂₆H₂₅N₃O₂: 412 (M+H).

4-[(2-Bromo-4-iodophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 8.10 (dd, 1H), 7.95 (s, 0.5H), 7.86-7.74 (m, 3.5H), 7.65-7.48 (m, 1.5H), 7.14-7.08 (m, 1.5H), 6.94 (d, 0.5H), 6.79 (s, 0.5H), 5.01-4.77 (m, 1H), 4.55-3.96 (m, 4H), 3.62-3.54 (m, 1H), 2.85-2.80 (m, 3H). MS (EI) for C₂₄H₁₉BrIN₃O₂: 588 (M+H).

4-{[7-(2-Methyl-1H-benzimidazol-6-yl)-2,3-dihydro-1,4-benzoxazepin-4(5H)-yl]carbonyl}-N-phenylbenzenesulfonamide. ¹H NMR (400 MHz, DMSO-d₆): δ 10.52-10.37 (m, 1H), 7.83-7.76 (m, 2H), 7.70-7.64 (m, 1H), 7.59-7.48 (m, 3.5H), 7.45-7.39 (m, 1.5H), 7.28-7.00 (m, 6H), 6.96-6.89 (m, 0.5H), 6.72 (s, 0.5H), 4.88-4.38 (m, 2H), 4.30-3.60 (m, 4H), 2.53 (s, 3H). MS (EI) for C₃₀H₂₆N₄O₄: 539 (M+H).

7-(2-Methyl-M-benzimidazol-6-yl)-4-[(2,4,5-trimethylphenyl)-carbonyl]-2,3,4,5-tetrahydro-1,4-benzoxazepine. ¹H NMR (400 MHz, DMSO-d₆): δ 12.27-12.14 (m, 1H), 7.73-7.33 (m, 4H), 7.20-6.97 (m, 3H), 6.86 (s, 0.5H), 6.69 (s, 0.5H), 6.56 (br s, 1H), 4.95-4.70 (m, 1H), 4.45-3.93 (m, 4H), 3.60-3.50 (m, 1H), 2.26-1.84 (m, 12H). MS (EI) for C₂₇H₂₇N₃O₂: 426 (M+H).

4-[(2,4-Dibromophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 8.00-7.92 (m, 1.5H), 7.83-7.74 (m, 2.5H), 7.66-7.49 (m, 2.5H), 7.26 (d, 0.5H), 7.12-7.07 (m, 1.5H), 6.82 (d, 0.5H), 4.99-4.77 (m, 1H), 4.54-3.93 (m, 4H), 3.61-3.51 (m, 1H), 2.81-2.76 (m, 3H). MS (EI) for C₂₄H₁₉N₃O₂: 542 (M+H).

7-(2-Methyl-1H-benzimidazol-6-yl)-4-({4-[(trifluoromethyl)-sulfonyl]phenyl}carbonyl)-2,3,4,5-tetrahydro-1,4-benzoxazepine. ¹H NMR (400 MHz, DMSO-d₆): δ 8.04-7.56 (m, 8H), 7.47-7.40 (m, 2H), 7.08 (d, 1H), 4.93 (br s, 2H), 4.38-4.01 (m, 4H), 2.80 (s, 3H). MS (EI) for C₂₅H₂₀F₃N₃O₄S: 516 (M+H).

4-(1-Benzothien-2-ylcarbonyl)-7-(2-methyl-1H-benzimidazol-6-yl)-2,3,4,5-tetrahydro-1,4-benzoxazepine. ¹H NMR (400 MHz, DMSO-d₆): δ 8.23-8.16 (m, 2H), 7.95 (s, 0.5H), 7.87-7.47 (m, 6H), 7.16-7.10 (m, 1H), 6.89 (s, 0.5H), 4.96-4.50 (m, 2H), 4.35-3.69 (m, 4H), 2.86-2.78 (m, 3H). MS (EI) for C₂₆H₂₁N₃O₂S: 440 (M+H for freebase form).

4-(1,3-Benzothiazol-6-ylcarbonyl)-7-(2-methyl-1H-benzimidazol-6-yl)-2,3,4,5-tetrahydro-1,4-benzoxazepine. ¹H NMR (400 MHz, DMSO-d₆): δ 9.49 (s, 1H), 8.38-7.68 (m, 5H), 7.65-7.48 (m, 3H), 7.18-6.94 (m, 1H), 4.90 (s, 1H), 4.50-3.75 (m, 5H), 2.81 (s, 3H). MS (EI) for C₂₅H₂₀N₄O₂S: 441 (M+H).

4-({4-[(Cyclopentylmethyl)sulfonyl]phenyl}carbonyl)-7-(2-methyl-1H-benzimidazol-6-yl)-2,3,4,5-tetrahydro-1,4-benzoxazepine. ¹H NMR (400 MHz, DMSO-d₆): δ 8.01-7.92 (m, 3H), 7.87-7.71 (m, 3H), 7.70-7.45 (m, 3H), 7.17-7.07 (m, 1H), 6.89-6.83 (m, 1H), 4.92 (s, 1H), 4.63-3.70 (m, 5H), 3.40 (d, 2H), 2.83 (d, 3H), 2.15-1.98 (m, 1H), 1.82-1.05 (m, 8H). MS (EI) for C₃₀H₃₁N₃O₄S: 530 (M+H).

7-(2-Methyl-1H-benzimidazol-6-yl)-4-{[1-(1-methylethyl)-1H-1,2,3-benzotriazol-5-yl]carbonyl}-2,3,4,5-tetrahydro-1,4-benzoxazepine. ¹H NMR (400 MHz, DMSO-d₆): δ 8.24-7.70 (m, 6H), 7.68-7.45 (m, 2H), 7.20-7.09 (m, 1H), 5.39-5.22 (m, 1H), 5.21-3.79 (m, 6H), 2.83 (s, 3H), 1.64 (d, 6H). MS (EI) for C₂₇H₂₆N₆O₂S: 467 (M+H for freebase form).

4-[(4-{[2,5-Bis(methyloxy)phenyl]sulfonyl}phenyl)carbonyl]-7-(2-methyl-1H-benzimidazol-6-yl)-2,3,4,5-tetrahydro-1,4-benzoxazepine. ¹H NMR (400 MHz, DMSO-d₆): δ 8.20-8.16 (d, 2H), 7.73-7.68 (m, 2H), 7.54-7.39 (m, 5H), 7.24-7.12 (m, 2H), 6.92-6.88 (m, 1H), 6.38-6.34 (m, 1H), 4.45-4.40 (s, 2H), 4.26-4.13 (m, 4H), 3.87 (s, 3H), 3.72 (s, 3H), 2.72 (s, 3H). MS (EI) for C₃₂H₂₉N₃O₆S: 584 (M+H).

4-(1,2,3-Benzothiadiazol-6-ylcarbonyl)-7-(2-methyl-1H-benzimidazol-6-yl)-2,3,4,5-tetrahydro-1,4-benzoxazepine. ¹H NMR (400 MHz, DMSO-d₆): δ 8.78-8.45 (m, 2H), 7.98-7.50 (m, 6H), 7.18-6.97 (m, 1H), 4.99-4.89 (m, 1H), 4.68-3.78 (m, 5H), 2.81 (s, 3H). MS (EI) for C₂₄H₁₉N₅O₂S: 442 (M+H).

4-{[4-(Cyclopentylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 7.98-7.90 (m, 3H), 7.87-7.46 (m, 6H), 7.16-6.78 (m, 1H), 4.94-4.89 (m, 1H), 4.56-4.03 (m, 4H), 3.87-3.70 (m, 2H), 2.80 (d, 3H), 1.90-1.42 (m, 8H). MS (EI) for C₂₉H₂₉N₃O₄S: 516 (M+H).

4-(1,3-Benzothiazol-2-ylcarbonyl)-7-(2-methyl-1H-benzimidazol-6-yl)-2,3,4,5-tetrahydro-1,4-benzoxazepine. ¹H NMR (400 MHz, DMSO-d₆): δ 8.25-8.12 (m, 2.5H), 8.06-7.55 (m, 7H), 7.20-7.10 (m, 0.5H), 5.55 (m, 1H), 5.50-4.30 (m, 4H), 4.16 (m, 1H), 2.81 (m, 3H). MS (EI) for C₂₅H₂₀N₄O₂S: 441 (M+H).

4-({4-[(4-Bromophenyl)sulfonyl]phenyl}carbonyl)-7-(2-methyl-1H-benzimidazol-6-yl)-2,3,4,5-tetrahydro-1,4-benzoxazepine. ¹H NMR (400 MHz, DMSO-d₆): δ 8.07-7.78 (m, 7H), 7.73-7.40 (m, 6H), 7.20-6.78 (m, 1H), 4.85 (m, 1H), 4.52-3.95 (m, 4H), 3.67 (m, 1H), 2.53 (s, 3H). MS (EI) for C₃₀H₂₄BrN₃O₄S: 602 (M+H).

4-[(1-Ethyl-5,7-difluoro-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.02-7.59 (m, 5H), 7.35-6.95 (m, 3.5H), 6.79-6.53 (m, 0.5H), 5.04-4.76 (m, 2H), 4.40-3.92 (m, 6H), 2.80 (s, 3H), 1.33-1.05 (m, 3H). MS (EI) for C₂₈H₂₄F₂N₄O₂: 287 (M+H).

4-[(4,6-Dichloro-1-ethyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.23 (bs, 2H), 7.85-7.27 (m, 7H), 7.23-6.48 (m, 2H), 4.99-4.78 (m, 2H), 4.44-3.95 (m, 6H), 2.51 (s, 3H), 1.15 (m, 3H). MS (EI) for C₂₈H₂₄Cl₂N₄O₂: 519 (M+H).

4-[(1-Ethyl-6-phenyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.04-7.58 (m, 9.5H), 7.55-7.32 (m, 4H), 7.20-7.09 (m, 1H), 6.80-6.50 (m, 0.5H), 4.95 (m, 2H), 4.50-4.00 (m, 6H), 2.79 (s, 3H), 1.32-1.00 (m, 3H). MS (EI) for C₃₄H₃₀N₄O₂: 527 (M+H).

4-[(1-Ethyl-7-methyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.10-7.70 (m, 4H), 7.65-7.59 (m, 1H), 7.55-7.37 (m, 1.5H), 7.20-6.94 (m, 3H), 6.70-6.40 (m, 0.5H), 5.10-4.70 (m, 2H), 4.44-3.96 (m, 6H), 2.80 (s, 3H), 2.74-2.63 (m, 3H), 1.26-1.02 (m, 3H). MS (EI) for C₂₉H₂₈N₄O₄: 465 (M+H).

4-{[1-Ethyl-4-(methyloxy)-1H-indol-2-yl]carbonyl}-7-(2-methyl-1H-benzimidazol-6-yl)-2,3,4,5-tetrahydro-1,4-benzoxazepine. ¹H NMR (400 MHz, DMSO-d₆): δ 8.00-7.54 (m, 5H), 7.38-7.05 (m, 4H), 6.70-6.54 (m, 2H), 4.90 (m, 2H), 4.42-4.00 (m, 6H), 2.79 (s, 3H), 1.12 (t, 3H). MS (EI) for C₂₉H₂₈N₄O₃: 481 (M+H).

4-[(7-Chloro-1-ethyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 7.99-7.73 (m, 4H), 7.30-7.22 (m, 2H), 7.18-6.52 (m, 4H), 7.02-6.93 (m, 1H), 6.85-6.65 (m, 1H), 5.00-4.72 (m, 2H), 4.54-3.89 (m, 6H), 2.78 (s, 3H), 1.28-1.07 (m, 3H). MS (EI) for C₂₈H₂₅ClN₄O₂: 485 (M+H).

7-(2-Methyl-1H-benzimidazol-6-yl)-4-(quinolin-7-ylcarbonyl)-2,3,4,5-tetrahydro-1,4-benzoxazepine. ¹H NMR (400 MHz, DMSO-d₆): δ 9.28-9.18 (m, 1H), 8.96-8.70 (m, 1H), 8.49-8.27 (m, 2H), 8.18-7.72 (m, 6H), 7.69-7.48 (m, 1H), 7.23-6.91 (m, 1H), 5.08-4.52 (m, 2H), 4.70-3.76 (m, 4H), 2.84 (s, 3H). MS (EI) for C₂₇H₂₂N₄O₂: 435 (M+H).

7-(2-Methyl-1H-benzimidazol-6-yl)-4-[(2-methyl-4-{[3-(methyloxy)phenyl]sulfonyl}phenyl)carbonyl]-2,3,4,5-tetrahydro-1,4-benzoxazepine. ¹H NMR (400 MHz, DMSO-d₆): δ 7.98-7.73 (m, 6H), 7.65-7.38 (m, 5H), 7.32-6.62 (m, 2H), 5.05-4.83 (m, 2H), 4.50-4.00 (m, 4H), 3.82 (d, 3H), 2.81 (d, 3H), 2.24-1.95 (d, 3H). MS (EI) for C₃₂H₂₉N₃O₅S: 568 (M+H).

7-(2-Methyl-1H-benzimidazol-6-yl)-4-(quinoxalin-6-ylcarbonyl)-2,3,4,5-tetrahydro-1,4-benzoxazepine. ¹H NMR (400 MHz, DMSO-d₆): δ 9.10-9.02 (m, 2H), 8.24-8.09 (m, 2H), 8.02-7.75 (m, 5H), 7.68-7.61 (m, 1H), 7.21-7.08 (m, 1H), 4.97 (m, 1H), 4.65 (m, 1H), 4.30-3.82 (m, 4H), 2.83 (s, 3H). MS (EI) for C₂₆H₂₁N₅O₂: 436 (M+H).

4-[(6-Chloro-1-ethyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.10-7.58 (m, 7H), 7.17-7.09 (m, 2H), 6.80-6.69 (m, 1H), 5.05-4.80 (m, 2H), 4.46-3.96 (m, 6H), 2.80 (s, 3H), 1.27-0.95 (m, 3H). MS (EI) for C₂₈H₂₅ClN₄O₂: 485 (M+H).

4-[(1-Ethyl-6-fluoro-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.10-7.67 (m, 6.5H), 7.53-7.40 (m, 1H), 7.20-7.08 (m, 1H), 7.02-6.93 (m, 1H), 6.85-6.65 (m, 0.5H), 5.00-4.86 (m, 2H), 4.46-4.03 (m, 6H), 2.81 (s, 3H), 1.28-1.00 (m, 3H). MS (EI) for C₂₈H₂₅FN₄O₂: 469 (M+H).

4-[(4-Chloro-1-ethyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.04-7.47 (m, 6H), 7.40-7.06 (m, 4H), 6.79-6.35 (m, 1H), 5.06-4.79 (m, 2H), 4.49-3.97 (m, 6H), 2.81 (s, 3H), 1.11 (t, 3H). MS (EI) for C₂₈H₂₅FN₄O₂: 469 (M+H).

4-[(4-{[3-Fluoro-4-(methyloxy)phenyl]-sulfonyl}phenyl)carbonyl]-7-(2-methyl-1H-benzimidazol-6-yl)-2,3,4,5-tetrahydro-1,4-benzoxazepine. ¹H NMR (400 MHz, DMSO-d₆): δ 8.00 (d, 2H), 7.95-7.70 (m, 5.5H), 7.63-7.29 (m, 4.5H), 7.13-7.05 (m, 1H), 4.90-3.62 (m, 9H), 2.81 (s, 3H). MS (EI) for C₃₁H₂₆FN₃O₅S: 572 (M+H).

7-(2-Methyl-1H-benzimidazol-6-yl)-4-[(4-methyl-4H-pyrrolo[2,3-d][1,3]thiazol-5-yl)carbonyl]-2,3,4,5-tetrahydro-1,4-benzoxazepine. ¹H NMR (400 MHz, CDCl₃): δ 8.61 (s, 1H), 7.55-7.28 (m, 4H), 7.11 (m, 2H), 6.90 (bs, 1H), 6.56 (s, 1H), 4.86 (m, 2H), 4.30-4.15 (m, 4H), 3.90 (s, 3H), 2.63 (s, 3H). MS (EI) for C₂₄H₂₁N₅O₂S: 444.2 (MH⁺).

7-(1-Methyl-1H-benzimidazol-5-yl)-4-[(4-methyl-4H-pyrrolo[2,3-d][1,3]thiazol-5-yl)carbonyl]-2,3,4,5-tetrahydro-1,4-benzoxazepine. ¹H NMR (400 MHz, CH₃OH-d₄) δ 8.72 (s, 1H), 8.05 (s, 1H), 7.69 (bs, 1H), 7.52-7.40 (m, 4H), 7.02 (m, 1H), 6.56 (s, 1H), 4.81 (s, 3H), 4.12 (m, 4H), 3.83 (s, 3H), 3.67 (m, 2H). MS (EI) for C₂₄H₂ᵢN₅O₂S: 444.2 (MH⁺).

4-[(4-Ethyl-4H-pyrrolo[2,3-d][1,3]thiazol-5-yl)carbonyl]-7-(1-methyl-1H-benzimidazol-5-yl)-2,3,4,5-tetrahydro-1,4-benzoxazepine. ¹H NMR (400 MHz, CH₃OH-d₄) δ 8.81 (s, 1H), 8.14 (s, 1H), 7.77 (bs, 1H), 7.60-7.45 (m, 4H), 7.10 (m, 1H), 6.67 (s, 1H), 4.93 (bs, 2H), 4.36-4.10 (m, 6H), 3.92 (s, 3H), 1.20 (bs, 3H). MS (EI) for C₂₅H₂₃N₅O₂S: 458.2 (MH⁺).

4-{[3-Fluoro-2-methyl-4-(methylsulfonyl)phenyl]carbonyl}-7-(2-methyl-1H-imidazo[4,5-b]pyridin-6-yl)-2,3,4,5-tetrahydro-1,4-benzoxazepine. ¹H NMR (400 MHz, CH₃OH-d₄) δ 8.54 (m, 0.5H), 8.33 (m, 0.5H), 8.09 (m, 0.5H), 7.90-7.79 (m, 1.5H), 7.70 (m, 0.5H), 7.58-7.51 (m, 1H), 7.26-7.12 (m, 2H), 6.69 (m, 0.5H), 5.06-4.90 (m, 1H), 4.58-4.32 (m, 2H), 4.25-4.0 (m, 2H), 3.73-3.65 (m, 1H), 3.24 (d, J_{FH}=18.4 Hz, 3H), 2.64 (s, 3H), 1.96 (s, 3H). MS (EI) for C₂₅H₂₃FN₄O₄S: 495.2 (MH⁺).

4-{[2-Ethyl-3-fluoro-4-(methylsulfonyl)phenyl]carbonyl}-7-(2-methyl-1H-imidazo[4,5-b]pyridin-6-yl)-2,3,4,5-tetrahydro-1,4-benzoxazepine. ¹H NMR (400 MHz, CH₃OH-d₄) δ 8.56 (m, 0.5H), 8.35 (m, 0.5H), 8.11 (m, 0.5H), 7.89-7.80 (m, 1.5H), 7.73-7.70 (m, 0.5H), 7.60-7.51 (m, 1H), 7.28-7.12 (m, 2H), 6.73 (m, 0.5H), 5.06-4.90 (m, 1H), 4.58-4.32 (m, 2H), 4.25-4.0 (m, 2H), 3.73-3.65 (m, 1H), 3.24 (d, J_{FH}=18.4 Hz, 3H), 2.64 (s, 3H), 1.96 (s, 3H). MS (EI) for C₂₅H₂₃FN₄O₄S: 495.2 (MH⁺).

4-{[2-Chloro-4-(methylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 8.09 (d, 1H), 7.98 (s, 0.5H), 7.96-7.89 (m, 1H), 7.87-7.78 (m, 2.5H), 7.74 (d, 0.5H), 7.66 (d, 1H), 7.64 (dd, 0.5H), 7.59 (dd, 0.5H), 7.49-7.45 (m, 1H), 7.12 (dd, 1H), 6.83 (d, 0.5H), 5.00 (d, 0.5H), 4.88 (d, 0.5H), 4.54 (d, 0.5H), 4.42 (d, 0.5H), 4.33-3.99 (m, 3H), 3.68-3.53 (m, 1H), 3.32 (d, 3H), 2.81 (d, 3H). MS (EI) for C₂₅H₂₂ClN₃O₄S: 496.0 (MH⁺).

7-(2-Methyl-1H-benzimidazol-6-yl)-4-{[1-methyl-5-(methyloxy)-1H-indol-2-yl]carbonyl}-2,3,4,5-tetrahydro-1,4-benzoxazepine. ¹H NMR (400 MHz, DMSO-d₆): δ 8.02-7.58 (m, 4H), 7.62 (dd, 1H), 7.40 (d, 1H), 7.13 (d, 1H), 7.05 (br s, 1H), 6.89 (dd, 1H), 6.68-6.32 (m, 1H), 4.91 (br s, 2H), 4.26 (br s, 2H), 4.09-4.02 (m, 2H), 3.75 (s, 3H), 3.70-3.35 (m, 3H), 2.80 (s, 3H). MS (EI) for C₂₈H₂₆N₄O₃: 467.1 (MH⁺).

4-[(1,5-Dimethyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.01-7.58 (m, 4H), 7.62 (dd, 1H), 7.50-7.32 (m, 1H), 7.13 (d, 1H), 7.02-6.86 (m, 2H), 6.64-6.35 (m, 1H), 5.00-4.70 (m, 2H), 4.40-4.18 (m, 2H), 4.14-3.94 (m, 2H), 3.93-3.64 (m, 3H), 2.81 (s, 3H), 2.76-2.54 (m, 3H). MS (EI) for C₂₈H₂₆N₄O₂: 451.1 (MH⁺).

4-[(5-Chloro-3-methyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 11.5 (s, 1H), 8.04-7.52 (m, 5H), 7.32 (d, 1H), 7.17 (dd, 1H), 7.10 (d, 1H), 4.84 (br s, 2H), 4.20 (br s, 2H), 3.95 (br s, 2H), 2.81 (s, 3H), 2.20 (br s, 3H). MS (EI) for C₂₇H₂₃ClN₄O₂: 471.1 (WO.

4-[(5,7-Difluoro-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.2 (s, 1H), 7.93 (s, 1H), 7.86-7.71 (m, 3H), 7.60 (dd, 1H), 7.26 (dd, 1H), 7.13-7.04 (m, 2H), 6.87 (br s, 1H), 4.95 (br s, 2H), 4.33 (br s, 2H), 4.16 (br s, 2H), 2.81 (s, 3H). MS (EI) for C₂₆H₂₀F₂N₄O₂: 459.1 (MH⁺).

4-[(4,6-Dichloro-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.2 (s, 1H), 7.93 (s, 1H), 7.85-7.67 (m, 3H), 7.61 (br s, 1H), 7.42 (s, 1H), 7.23 (br s, 1H), 7.10 (br s, 1H), 6.94-6.65 (m, 1H), 5.18-4.90 (m, 2H), 4.40 (br s, 2H), 4.34-3.96 (m, 2H), 2.80 (s, 3H). MS (EI) for C₂₆H₂₀Cl₂N₄O₂: 491.0 (MH⁺).

4-{[2-Bromo-4-(ethylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 8.15 (d, 1H), 7.98 (s, 0.5H), 7.95 (dd, 0.5H), 7.90 (dd, 0.5H), 7.87-7.82 (m, 1H), 7.82-7.78 (m, 1H), 7.74 (d, 0.5H), 7.64 (dd, 0.5H), 7.62 (d, 0.5H), 7.58 (dd, 0.5H), 7.46 (dd, 0.5H), 7.43 (d, 0.5H), 7.13 (dd, 1H), 6.77 (d, 0.5H), 5.02 (d, 0.5H), 4.87 (d, 0.5H), 4.53 (d, 0.5H), 4.39 (d, 0.5H), 4.35-3.98 (m, 3H), 3.66-3.54 (m, 1H), 3.47-3.32 (m, 2H), 2.81 (d, 3H), 1.13 (t, 1.5H), 1.03 (t, 1.5H). MS (EI) for C₂₆H₂₄BrN₃O₄S: 554.0 (MH⁺).

1,1,1,3,3,3-Hexafluoro-2-(4-{[7-(2-methyl-1H-benzimidazol-6-yl)-2,3-dihydro-1,4-benzoxazepin-4(5H)-yl]carbonyl}phenyl)propan-2-ol. ¹H NMR (400 MHz, DMSO-d₆): δ 8.92 (br s, 1H), 7.99-7.65 (m, 5.5H), 7.64-7.35 (m, 3H), 7.18-7.06 (m, 1H), 6.71 (br s, 0.5H), 4.90 (br s, 1.5H), 4.53 (br s, 0.5H), 4.29 (br s, 0.5H), 4.21 (br s, 1.5H), 4.05 (br s, 0.5H), 3.76 (br s, 1.5H), 2.81 (s, 3H). MS (EI) for C₂₇H₂ᵢF₆N₃O₃: 550.0 (MH⁺).

4-[(4-Chloro-2-methylphenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.95 (s, 0.5H), 7.84-7.72 (m, 2.5H), 7.59 (dd, 0.5H), 7.55 (dd, 0.5H), 7.51 (dd, 0.5H), 7.35 (dd, 1H), 7.28 (dt, 1H), 7.15 (d, 0.5H), 7.09 (t, 1H), 7.05 (d, 0.5H), 6.74 (d, 0.5H), 4.90 (br d, 1H), 4.43 (br s, 1H), 4.25 (br s, 1H), 4.15-4.08 (m, 1H), 4.05 (br s, 1H), 3.55 (br s, 1H), 2.79 (d, 3H), 2.11 (s, 1.5H), 1.89 (s, 1.5H). MS (EI) for C₂₅H₂₂ClN₃O₂: 432.0, 434.0 (MH⁺).

7-(2-Methyl-1H-benzimidazol-6-yl)-4-{[4-(1-methylethyl)-phenyl]carbonyl}-2,3,4,5-tetrahydro-1,4-benzoxazepine hydrochloride salt. ¹H NMR (400 MHz, CD₃OD): δ 7.95-7.70 (m, 3H), 7.65-7.53 (m, 1.5H), 7.36-7.11 (m, 5H), 6.82 (br s, 0.5H), 4.62 (br s, 1H), 4.30 (br s, 1H), 4.16-4.09 (m, 2.5H), 3.94-3.87 (br s, 1.5H), 3.02-2.89 (m, 1H), 2.87 (s, 3H), 1.25 (d, 6H). MS (EI) for C₂₇H₂₇N₃O₂: 426.1 (MH⁺).

4-{[2-Bromo-3-fluoro-4-(methylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 7.97 (s, 0.5H), 7.91-7.78 (m, 3H), 7.73 (d, 0.5H), 7.64 (dd, 0.5H), 7.59 (dd, 0.5H), 7.48 (dd, 0.5H), 7.43 (dd, 0.5H), 7.26 (dd, 0.5H), 7.13 (t, 1H), 6.97 (d, 0.5H), 5.00 (d, 0.5H), 4.88 (d, 0.5H), 4.53 (d, 0.5H), 4.47 (d, 0.5H), 4.29 (t, 1H), 4.24-4.01 (m, 2H), 3.68-3.56 (m, 1H), 3.38 (d, 3H), 2.81 (d, 3H). MS (EI) for C₂₅H₂₁BrN₃O₄S: 558.0 (MH⁺).

7-(2-Methyl-1H-benzimidazol-6-yl)-4-({4-[(4-methylphenyl)-sulfonyl]phenyl}carbonyl)-2,3,4,5-tetrahydro-1,4-benzoxazepine. ¹H NMR (400 MHz, DMSO-d₆): δ 7.99 (d, 2H), 7.94 (s, 1H), 7.90-7.73 (m, 5H), 7.60 (d, 2H), 7.52-7.36 (m, 2.5H), 7.15-7.08 (m, 1H), 6.87 (s, 0.5H), 4.88 (br s, 1H), 4.53 (br s, 1H), 4.31 (br s, 1H), 4.15 (br s, 1H), 4.02 (br s, 1H), 3.68 (br s, 1H), 2.80 (s, 3H), 2.39-2.32 (m, 3H). MS (EI) for C₃₁H₂₇N₃O₄S: 538.0 (MH⁺).

7-(2-Methyl-1H-benzimidazol-6-yl)-44 {4-[(4-methylphenyl)-thio]phenyl}carbonyl)-2,3,4,5-tetrahydro-1,4-benzoxazepine. ¹H NMR (400 MHz, DMSO-d₆): δ 7.98-7.70 (m, 3.5H), 7.59 (dd, 1.5H), 7.45-7.00 (m, 9H), 4.85 (br s, 1H), 4.59 (br s, 1H), 4.30 (br s, 1H), 4.17 (br s, 1H), 3.99 (br s, 1H), 3.78 (br s, 1H), 2.79 (s, 3H), 2.37-2.24 (m, 3H). MS (EI) for C₃₁H₂₇N₃O₂S: 506.1 (MH⁺).

4-({4-[(3-Fluorophenyl)sulfonyl]-2-methylphenyl}carbonyl)-7-(2-methyl-1H-benzimidazol-6-yl)-2,3,4,5-tetrahydro-1,4-benzoxazepine. ¹H NMR (400 MHz, DMSO-d₆): δ 7.95-7.92 (m, 1.5H), 7.89-7.75 (m, 4.5H), 7.74 (dd, 1H), 7.72-7.48 (m, 3H), 7.40 (d, 0.5H), 7.38 (dd, 0.5H), 7.29 (d, 0.5H), 7.09 (dd, 1H), 6.61 (s, 0.5H), 5.00-4.80 (m, 1H), 4.46-3.96 (m, 4H), 3.57-3.42 (m, 1H), 2.79 (d, 3H), 2.19 (s, 1.5H), 1.95 (s, 1.5H). MS (EI) for C₃₁H₂₆FN₃O₄S: 556.0 (MH⁺).

4-[(3-Fluoro-4-{[4-(methyloxy)phenyl]sulfonyl}phenyl)-carbonyl]-7-(2-methyl-1H-benzimidazol-6-yl)-2,3,4,5-tetrahydro-1,4-benzoxazepine. ¹H NMR (400 MHz, DMSO-d₆): δ 8.09-8.00 (m, 1H), 7.96-7.73 (m, 5.5H), 7.64-7.57 (m, 1H), 7.56-7.49 (m, 1H), 7.47 (d, 0.5H), 7.30 (t, 0.5H), 7.22-7.08 (m, 3H), 7.04 (br s, 0.5H), 4.87 (br s, 1.5H), 4.56 (br s, 0.5H), 4.32 (br s, 0.5H), 4.18-4.12 (m, 1.5H), 4.04-3.98 (m, 0.5H), 3.89-3.80 (m, 3H), 3.74-3.67 (m, 1.5H), 2.81 (s, 3H). MS (EI) for C₃₁H₂₆FN₃O₅S: 572.1 (MH⁺).

4-{[2-Bromo-4-(methylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 8.21 (dd, 1H), 8.00-7.93 (m, 1.5H), 7.87-7.81 (m, 2H), 7.79 (d, 0.5H), 7.64 (dd, 0.5H), 7.61 (d, 0.5H), 7.59 (dd, 0.5H), 7.47 (dd, 0.5H), 7.42 (d, 0.5H), 7.13 (dd, 1H), 6.81 (d, 0.5H), 5.02 (d, 0.5H), 4.86 (d, 0.5H), 4.53 (d, 0.5H), 4.40 (d, 0.5H), 4.32-3.98 (m, 3H), 3.66-3.55 (m, 1H), 3.32 (d, 3H), 2.82 (d, 3H). MS (EI) for C₂₅H₂₂BrN₃O₄S: 540.0 (MH⁺).

4-{[1-Ethyl-6-(methyloxy)-1H-indol-2-yl]carbonyl}-7-(2-methyl-1H-benzimidazol-6-yl)-2,3,4,5-tetrahydro-1,4-benzoxazepine. ¹H NMR (400 MHz, DMSO-d₆): δ 7.96-7.50 (m, 4H), 7.58 (dd, 1H), 7.44 (d, 1H), 7.09 (d, 1H), 6.99 (d, 1H), 6.73 (dd, 1H), 6.56 (br s, 1H), 4.90 (br s, 2H), 4.28 (br s, 2H), 4.18-4.09 (m, 2H), 4.08-4.02 (m, 2H), 3.78 (s, 3H), 2.78 (s, 3H), 1.09 (br s, 3H). MS (EI) for C₂₉H₂₈N₄O₃: 481.1 (MH⁺).

4-[(1-Ethyl-5-fluoro-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 8.01-7.51 (m, 5.5H), 7.36 (d, 1H), 7.11 (dd, 2H), 7.08 (d, 0.5H), 6.75-6.45 (m, 1H), 5.01-4.81 (m, 2H), 4.40-4.22 (m, 2H), 4.17 (br s, 2H), 4.04 (br s, 2H), 2.80 (s, 3H), 1.27-0.95 (m, 3H). MS (EI) for C₂₈H₂₅FN₄O₂: 469.1 (MH⁺).

7-(2-Methyl-1H-benzimidazol-6-yl)-4-{[3-methyl-5-(methylsulfonyl)-2-thienyl]carbonyl}-2,3,4,5-tetrahydro-1,4-benzoxazepine. ¹H NMR (400 MHz, DMSO-d₆): δ 13.1 (br s, 0.5H), 12.0 (br s, 0.5H), 8.44 (s, 1H), 7.78-7.32 (m, 5H), 7.07 (d, 1H), 4.92-4.54 (m, 2H), 4.21 (br s, 2H), 4.12-3.72 (m, 2H), 3.55-2.90 (m, 3H), 2.57 (s, 3H), 2.39-1.94 (m, 3H). MS (EI) for C₂₄H₂₃N₃O₄S₂: 482.0 (MH⁺).

4-[(4-Iodo-2-methylphenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.96 (br s, 1H), 7.85-7.55 (m, 6H), 7.4 (dd, 0.5H), 7.11 (dd, 1H), 6.95 (d, 0.5H), 6.85 (d, 0.5H), 6.70 (d, 0.5H), 4.90 (br d, 1H), 4.48-3.98 (m, 4H), 3.57 (br s, 1H), 2.81 (d, 3H), 2.10 (s, 1.5H), 1.85 (s, 1.5H). MS (EI) for C₂₅H₂₂IN₃O₂: 524.0 (MH+).

4-{[2-Ethyl-4-(ethylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 7.99-7.97 (m, 0.5H), 7.88-7.72 (m, 4.5H), 7.63 (dd, 0.5H), 7.57 (dd, 0.5H), 7.45 (dd, 0.5H), 7.43 (d, 0.5H), 7.30 (d, 0.5H), 7.12 (dd, 1H), 6.64 (d, 0.5H), 5.05 (d, 0.5H), 4.88 (d, 0.5H), 4.51-4.37 (m, 1H), 4.37-4.02 (m, 3H), 3.62-3.52 (m, 1H), 3.34 (q, 1H), 3.29 (q, 1H), 2.81 (d, 3H), 2.69-2.43 (m, 1H), 2.43-2.18 (m, 1H), 1.13 (q, 3H), 1.01 (dt, 3H). MS (EI) for C₂₈H₂₉N₃O₄S: 504.1 (MH⁺).

N,N-Diethyl-4-[(4-{[7-(2-methyl-1H-benzimidazol-6-yl)-2,3-dihydro-1,4-benzoxazepin-4(5H)-yl]carbonyl}phenyl)sulfonyl]aniline. ¹H NMR (400 MHz, CDCl₃): δ 10.5 (br s, 1H), 8.09 (d, 1.8H), 7.92 (d, 0.2H), 7.74 (d, 2H), 7.66 (d, 1H), 7.51 (dd, 1H), 7.38 (br d, 3H), 7.19 (br s, 1H), 7.12 (d, 1H), 6.67-6.62 (m, 2H), 6.34 (br s, 1H), 4.86 (br s, 0.2H), 4.41 (s, 1.8H), 4.23-4.17 (m, 1.8H), 4.17-4.11 (m, 1.8H), 4.04 (br s, 0.2H), 3.75 (br s, 0.2H), 3.39 (q, 4H), 2.66 (s, 3H), 1.18 (t, 6H). MS (EI) for C₃₄H₃₄N₄O₄S: 595.3 (MH⁺).

7-(2-Methyl-1H-benzimidazol-6-yl)-4-{[4-(morpholin-4-ylsulfonyl)phenyl]carbonyl}-2,3,4,5-tetrahydro-1,4-benzoxazepine. ¹H NMR (400 MHz, DMSO-d₆): δ 12.24 (s, 1H), 7.86-7.75 (m, 2H), 7.74-7.63 (m, 2H), 7.58-7.37 (m, 4H), 7.26-6.80 (m, 2H), 4.88 (s, 1H), 4.50 (s, 1H), 4.27 (s, 1H), 4.05 (s, 1H), 3.74 (s, 1H), 3.67-3.51 (m, 4H), 3.01-2.83 (m, 4H); MS (EI) for C₂₈H₂₈N₄O₅S: 533.2 (MH+).

N-Methyl-4-{[7-(2-methyl-1H-benzimidazol-6-yl)-2,3-dihydro-1,4-benzoxazepin-4(5H)-yl]carbonyl}-N-(methyloxy)benzenesulfonamide. ¹H NMR (400 MHz, DMSO-d₆): δ 12.25 (s, 1H), 7.91-7.84 (m, 2H), 7.73-7.64 (m, 2H), 7.58-7.37 (m, 4H), 7.17-6.72 (m, 2H), 4.88 (s, 1H), 4.50 (s, 1H), 4.27 (s, 1H), 4.15 (s, 1H), 4.06 (s, 1H), 3.77-3.64 (m, 4H), 2.80-2.68 (m, 3H); MS (EI) for C₂₆H₂₆N₄O₅S: 507.2 (MH+).

7-(2-Methyl-1H-benzimidazol-6-yl)-4-{[4-(pyrrolidin-1-ylsulfonyl)phenyl]carbonyl}-2,3,4,5-tetrahydro-1,4-benzoxazepine. ¹H NMR (400 MHz, DMSO-d₆): δ 12.40-11.92 (m, 1H), 7.89-7.82 (m, 2H), 7.72-7.59 (m, 2H), 7.56-7.36 (m, 4H), 7.20-6.55 (m, 2H), 4.87 (s, 1H), 4.50 (s, 1H), 4.28 (s, 1H), 4.16 (s, 1H), 4.05 (s, 1H), 3.72 (s, 1H), 3.20 (m, 4H), 1.72-1.46 (m, 4H); MS (EI) for C₂₈H₂₈N₄O₄S: 517.2 (MH+).

7-(2-Methyl-1H-benzimidazol-6-yl)-4-({4-[(4-methylpiperazin-1-yl) sulfonyl]phenyl}carbonyl)-2,3,4,5-tetrahydro-1,4-benzoxazepine. ¹H NMR (400 MHz, DMSO-d₆): δ 10.68 (s, 1H), 8.02-7.48 (m, 9H), 7.18-7.08 (m, 1H), 4.91 (s, 1H), 4.55 (s, 1H), 4.34 (s, 1H), 4.18 (s, 1H), 4.06 (s, 1H), 3.83-3.68 (m, 3H), 3.24-3.04 (m, 2H), 2.88-2.60 (m, 7H); MS (EI) for C₂₉H₃₁N₅O₄S: 546.2 (MH+).

7-(2-Methyl-1H-benzimidazol-6-yl)-4-{[4-(piperidin-1-ylsulfonyl)phenyl]carbonyl}-2,3,4,5-tetrahydro-1,4-benzoxazepine. ¹H NMR (400 MHz, DMSO-d₆): δ 12.21 (s, 1H), 7.82-7.35 (m, 8H), 7.23-6.75 (m, 2H), 4.88 (s, 1H), 4.50 (s, 1H), 4.28 (s, 1H), 4.16 (s, 1H), 4.05 (s, 1H), 3.72 (s, 1H), 2.96-2.77 (m, 4H), 1.61-1.17 (m, 6H); MS (EI) for C₂₉H₃₀N₄O₄S: 531.2 (MH+).

N,N-Dimethyl-4-{[7-(2-methyl-1H-benzimidazol-6-yl)-2,3-dihydro-1,4-benzoxazepin-4(5H)-yl]carbonyl}benzenesulfonamide. ¹H NMR (400 MHz, DMSO-d₆): δ 12.31-12.16 (m, 1H), 7.85-7.35 (m, 8H), 7.24-6.73 (m, 2H), 4.87 (s, 1H), 4.50 (s, 1H), 4.27 (s, 1H), 4.15 (s, 1H) 4.05 (s, 1H), 3.73 (s, 1H), 2.63 (s, 3H), 2.54 (s, 3H); MS (EI) for C₂₆H₂₆N₄O₄S: 491.2 (MH+).

N-(Furan-2-ylmethyl)-4-{[7-(2-methyl-1H-benzimidazol-6-yl)-2,3-dihydro-1,4-benzoxazepin-4(5H)-yl]carbonyl}benzenesulfonamide. ¹H NMR (400 MHz, DMSO-d₆): δ 11.99 (s, 1H), 8.61-8.30 (m, 1H), 7.68-7.66 (m, 3H), 7.54-7.23 (m, 6H), 7.14-6.76 (m, 1H), 6.31 (m, 1H), 6.17-6.05 (m, 1H), 4.87 (s, 1H), 4.51 (s, 1H), 4.27 (s, 1H), 4.17 (m, 1H), 4.04 (m, 3H), 3.70- (s, 1H), 2.61-2.52 (m, 3H); MS (EI) for C₂₉H₂₆N₄O₅S: 543.2 (MH+).

4-{[7-(2-Methyl-1H-benzimidazol-6-yl)-2,3-dihydro-1,4-benzoxazepin-4(5H)-yl]carbonyl}-N-prop-2-en-1-ylbenzenesulfonamide. ¹H NMR (400 MHz, DMSO-d₆): δ 12.24 (s, 1H), 8.02-7.34 (m, 8H), 7.24-6.67 (m, 2H), 5.75-5.51 (m, 1H), 5.20-4.91 (m, 2H), 4.87 (s, 1H), 4.52 (s, 1H), 4.26 (s, 1H), 4.15 (s, 1H), 4.02 (s, 1H), 3.72 (s, 1H), 3.43 (m, 2H); MS (EI) for C₂₇H₂₆N₄O₄S: 503.2 (MH+).

4-{[7-(2-Methyl-1H-benzimidazol-6-yl)-2,3-dihydro-1,4-benzoxazepin-4(5H)-yl]carbonyl}-N-prop-2-yn-1-ylbenzenesulfonamide. ¹H NMR (400 MHz, DMSO-d₆): δ 12.24 (s, 1H), 7.92-7.81 (m, 2H), 7.76-7.18 (m, 7H), 7.12-6.75 (m, 1H), 4.87 (s, 1H), 4.49 (s, 1H), 4.25 (s, 1H), 4.15 (s, 1H), 4.04 (s, 1H), 3.84-3.66 (m, 3H), 3.05 (s, 1H); MS (EI) for C₂₇H₂₄N₄O₄S: 501.2 (MH+).

4-{[7-(2-Methyl-1H-benzimidazol-6-yl)-2,3-dihydro-1,4-benzoxazepin-4(5H)-yl]carbonyl}-N-(1-methylethyl)benzenesulfonamide. ¹H NMR (400 MHz, DMSO-d₆): δ 12.38 (s, 1H), 8.09-7.35 (m, 9H), 7.24-6.64 (m, 2H), 4.87 (s, 1H), 4.50 (s, 1H), 4.26 (s, 1H), 4.15 (s, 1H), 4.04 (s, 1H), 3.72 (s, 1H), 1.02-0.81 (m, 6H); MS (EI) for C₂₄H₂₈N₄O₄S: 505.2 (MH+).

4-[(1,3-Dimethyl-1H-pyrazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.55-11.94 (m, 1H), 7.73-6.93 (m, 6H), 6.59-6.04 (m, 1H), 4.84 (s, 1H), 4.30-4.15 (m, 2H), 4.05-3.86 (m, 2H), 3.73-3.43 (m, 3H), 2.45-2.10 (m, 3H); MS (EI) for C₂₃H₂₃N₅O₂: 402.2 (MH+).

4-[(4-Bromo-1-ethyl-3-methyl-1H-pyrazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.25 (s, 1H), 7.67 (d, 1H), 7.59-7.34 (m, 3H), 7.27-6.75 (m, 2H), 4.9 (m, 1H), 4.57 (m, 1H), 4.41-3.52 (m, 6H), 2.26-2.11 (m, 3H), 1.27-0.94 (m, 3H); MS (EI) for C₂₄H₂₄BrN₅O₂: 494.1 (M+).

7-(2-Methyl-1H-benzimidazol-6-yl)-4-({1-[2-(methyloxy)ethyl]-1H-indol-2-yl}carbonyl)-2,3,4,5-tetrahydro-1,4-benzoxazepine. ¹H NMR (400 MHz, DMSO-d₆): δ 12.24 (s, 1H), 7.80-7.03 (m, 10H), 6.76-6.39 (m, 1H), 4.96-4.76 (m, 2H), 4.46-4.18 (m, 4H), 4.07 (s, 2H), 3.47-3.39 (m, 2H), 3.12-2.94 (m, 3H); MS (EI) for C₂₉H₂₈N₄O₃: 481.2 (MH+).

4-{[1-(4-Chlorophenyl)-4-propyl-1H-pyrazol-3-yl]carbonyl}-7-(2-methyl-1H-benzimidazol-6-yl)-2,3,4,5-tetrahydro-1,4-benzoxazepine. ¹H NMR (400 MHz, DMSO-d₆): δ 12.23 (s, 1H), 7.83-6.90 (m, 11H), 4.78 (s, 2H), 4.20 (s, 2H), 3.97 (m, 2H), 2.72-2.58 (m, 1H), 2.44-2.28 (m, 1H), 1.33-1.14 (m, 2H), 0.99-0.78 (1H); MS (EI) for C₃₀H₂₈ClN₅O₂: 526.0 (M+).

N-(1,1-Dimethylethyl)-4-{[7-(2-methyl-1H-benzimidazol-6-yl)-2,3-dihydro-1,4-benzoxazepin-4(5H)-yl]carbonyl}benzenesulfonamide. ¹H NMR (400 MHz, DMSO-d₆): δ 12.23 (s, 1H), 7.91-7.83 (m, 2H), 7.78-7.34 (m, 7H), 7.17-6.67 (m, 2H), 4.87 (s, 1H), 4.50 (s, 1H), 4.26 (s, 1H), 4.16 (s, 1H), 4.04 (s, 1H), 3.71 (s, 1H), 1.10-0.95 (m, 9H); MS (EI) for C₂₈H₃₀N₄O₄S: 519.2 (MH+).

7-(2-Methyl-1H-benzimidazol-6-yl)-4-({1-[2-(methyloxy)ethyl]-1H-indol-2-yl}carbonyl)-2,3,4,5-tetrahydro-1,4-benzoxazepine. ¹H NMR (400 MHz, DMSO-d₆): δ 12.24 (s, 1H), 7.80-7.03 (m, 10H), 6.76-6.39 (m, 1H), 4.96-4.76 (m, 2H), 4.46-4.18 (m, 4H), 4.07 (s, 2H), 3.47-3.39 (m, 2H), 3.12-2.94 (m, 3H); MS (EI) for C₂₉H₂₈N₄O₃: 481.2 (MH+).

4-({5-[(2,2-Difluoroethyl)oxy]-1-methyl-1H-pyrazol-4-yl}carbonyl)-7-(2-methyl-1H-benzimidazol-6-yl)-2,3,4,5-tetrahydro-1,4-benzoxazepine. ¹H NMR (400 MHz, DMSO-d₆): δ 7.76-7.10 (m, 6H), 7.02-6.96 (m, 1H), 4.76 (s, 2H), 4.50-4.11 (m, 4H), 3.93 (s, 2H), 3.60 (s, 3H); MS (EI) for C₂₄H₂₃F₂N₅O₃: 468.2 (MH+).

4-[(1-Cyclopropyl-2,5-dimethyl-1H-pyrrol-3-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.24 (s, 1H), 7.73-7.27 (m, 5H), 7.05-6.97 (m, 1H), 5.72 (s, 1H), 4.69 (s, 2H), 4.14 (s, 2H), 3.90 (s, 2H), 2.93 (s, 1H), 2.26-2.09 (m, 6H), 0.97 (m, 2H), 0.82 (s, 2H); MS (EI) for C₂₇H₂₈N₄O₂: 441.2 (MH+).

7-(2-Methyl-1H-benzimidazol-6-yl)-4-[(1-methyl-3-propyl-1H-pyrazol-5-yl)carbonyl]-2,3,4,5-tetrahydro-1,4-benzoxazepine. ¹H NMR (400 MHz, DMSO-d₆): δ 12.30-12.19 (m, 1H), 7.76-6.95 (m, 6H), 6.33-6.02 (m, 1H), 4.84 (s, 1H), 4.67 (s, 1H), 4.32-4.16 (m, 2H), 4.04-3.86 (m, 2H), 3.73-3.48 (m, 3H), 1.64-1.43 (m, 2H), 0.95-0.72 (m, 3H); MS (EI) for C₂₅H₂₇N₅O₂: 430.2 (MH+).

N,1-Dimethyl-5-{[7-(2-methyl-1H-benzimidazol-6-yl)-2,3-dihydro-1,4-benzoxazepin-4(5H)-yl]carbonyl}-1H-pyrrole-3-sulfonamide. ¹H NMR (400 MHz, DMSO-d₆): δ 11.99 (s, 1H), 7.66 (s, 1H), 7.56-7.36 (m, 4H), 7.12-6.90 (m, 2H), 6.49 (s, 1H), 4.83 (s, 2H), 4.27 (s, 2H), 4.01 (s, 2H), 3.60 (s, 3H), 2.52 (s, 3H), 2.34 (s, 3H); MS (EI) for C₂₄H₂₅N₅O₄S: 480.2 (MH+).

N,N-Diethyl-1-methyl-5-{[7-(2-methyl-1H-benzimidazol-6-yl)-2,3-dihydro-1,4-benzoxazepin-4(5H)-yl]carbonyl}-1H-pyrrole-3-sulfonamide. ¹H NMR (400 MHz, DMSO-d₆): δ 12.33-11.95 (m, 1H), 7.77-6.97 (m, 7H), 6.67-6.28 (m, 1H), 4.81 (s, 2H), 4.28 (s, 2H), 3.99 (s, 2H), 3.58 (s, 3H), 3.17-2.76 (m, 4H), 1.20-0.71 (m, 6H); MS (EI) for C₂₇H₃ᵢN₅O₄S: 522.2 (MH+).

7-(2-Methyl-1H-benzimidazol-6-yl)-4-{[1-methyl-4-(pyrrolidin-1-ylsulfonyl)-1H-pyrrol-2-yl]carbonyl}-2,3,4,5-tetrahydro-1,4-benzoxazepine. ¹H NMR (400 MHz, DMSO-d₆): δ 12.22 (s, 1H), 7.78-7.02 (m, 7H), 6.76-6.30 (m, 1H), 4.82 (s, 2H), 3.99 (s, 2H), 3.61 (s, 3H), 3.17-2.81 (m, 4H), 1.76-1.38 (m, 4H); MS (EI) for C₂₇H₂₉N₅O₄S: 520.2 (MH+).

7-(2-Methyl-1H-benzimidazol-6-yl)-4-{[1-methyl-4-(morpholin-4-ylsulfonyl)-1H-pyrrol-2-yl]carbonyl}-2,3,4,5-tetrahydro-1,4-benzoxazepine. ¹H NMR (400 MHz, DMSO-d₆): δ 12.54-11.91 (m, 1H), 7.78-7.00 (m, 7H), 6.72-6.16 (m, 1H), 4.85 (s, 2H), 4.31 (s, 2H), 3.99 (s, 2H), 3.64 (s, 4H), 3.54-3.33 (m, 3H), 2.90-2.58 (m, 4H); MS (EI) for C₂₇H₂₉N₅O₅S: 536.2 (MH+).

1-Methyl-5-{[7-(2-methyl-1H-benzimidazol-6-yl)-2,3-dihydro-1,4-benzoxazepin-4(5H)-yl]carbonyl}-1H-pyrrole-2-sulfonamide. ¹H NMR (400 MHz, DMSO-d₆): δ 12.45-11.95 (m, 1H), 7.65 (s, 1H), 7.55-7.37 (m, 5H), 7.09-6.98 (m, 3H), 6.56 (s, 1H), 4.81 (s, 2H), 4.25 (s, 2H), 4.02 (s, 2H), 3.60 (s, 3H); MS (EI) for C₂₃H₂₃N₅O₄S: 466.2 (MH+).

4-[(3-Chloro-1-ethyl-1H-indol-2-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.34-12.09 (m, 1H), 7.81-7.01 (m, 9H), 6.92-6.58 (m, 1H), 5.08-4.88 (m, 1H), 4.97 (m, 1H), 4.45-4.26 (m, 1H), 4.23-3.69 (m, 5H), 2.46 (s, 2H), 1.29-0.92 (m, 3H); MS (EI) for C₂₈H₂₅ClN₄O₂: 485.2 (MH+).

7-(2-Methyl-1H-benzimidazol-6-yl)-4-{[4-(5-methyl-1H-pyrazol-1-yl)phenyl]carbonyl}-2,3,4,5-tetrahydro-1,4-benzoxazepine. ¹H NMR (400 MHz, DMSO-d₆): δ 12.23 (s, 1H), 8.64 (s, 1H), 7.93-6.89 (m, 10H), 6.38 (s, 1H), 4.92-4.55 (m, 2H), 4.35-3.76 (m, 4H), 2.28 (s, 3H); MS (EI) for C₂₈H₂₅N₅O₂: 464.2 (MH+).

7-(2-Methyl-1H-benzimidazol-6-yl)-4-{[4-(4-methylpiperazin-1-yl)phenyl]carbonyl}-2,3,4,5-tetrahydro-1,4-benzoxazepine. ¹H NMR (400 MHz, DMSO-d₆): δ 11.97 (s, 1H), 7.87-6.91 (m, 10H), 4.89-4.57 (m, 2H), 4.29-3.78 (m, 4H), 2.98-2.81 (m, 4H), 2.68-2.54 (m, 3H); MS (EI) for C₂₉H₃ᵢN₅O₂: 482.3 (MH+).

7-(2-Methyl-1H-benzimidazol-6-yl)-4-[(4-morpholin-4-ylphenyl)carbonyl]-2,3,4,5-tetrahydro-1,4-benzoxazepine. ¹H NMR (400 MHz, DMSO-d₆): δ 12.27 (s, 1H), 7.70-7.17 (m, 7H), 7.08-6.90 (m, 3H), 4.72 (s, 2H), 4.19 (s, 2H), 3.90 (s, 2H), 3.74 (m, 4H), 3.18 (m, 4H); MS (EI) for C₂₈H₂₈N₄O₃: 469.2 (MH+).

4-(2,1-Benzisoxazol-3-ylcarbonyl)-7-(2-methyl-1H-benzimidazol-6-yl)-2,3,4,5-tetrahydro-1,4-benzoxazepine. ¹H NMR (400 MHz, DMSO-d₆): δ 12.26 (m, 1H), 7.87-7.02 (m, 10H), 5.03-4.91 (m, 2H), 4.38-4.08 (m, 4H); MS (EI) for C₂₅H₂₀N₄O₃: 425.0 (MH+).

7-(2-Methyl-1H-benzimidazol-6-yl)-4-{[4-(1,2,3-thiadiazol-4-yl)phenyl]carbonyl}-2,3,4,5-tetrahydro-1,4-benzoxazepine. ¹H NMR (400 MHz, CDCl₃); δ 8.38 (s, 1H), 8.24-8.09 (m, 2H), 7.77-7.58 (m, 1H), 7.56-7.47 (m, 5H), 7.40-7.34 (m, 1H), 7.18-7.11 (m, 1H), 6.59 (s, 1H), 4.48 (s, 2H), 4.32-4.09 (m, 4H), 2.76 (s, 3H); MS (EI) for C₂₆H₂ᵢN₅O₂S: 468.2 (MH+).

4-(1H-Imidazol-4-ylcarbonyl)-7-(2-methyl-1H-benzimidazol-6-yl)-2,3,4,5-tetrahydro-1,4-benzoxazepine. ¹H NMR (400 MHz, DMSO-d₆): δ 12.51 (s, 1H), 12.24 (s, 1H), 7.88-7.18 (m, 7H), 7.09-6.98 (m, 1H), 5.53 (s, 1H), 4.81-4.55 (m, 2H), 4.34-3.92 (m, 3H); MS (EI) for C₂₁H₁₉N₅O₂: 374.2 (MH+).

7-(2-Methyl-1H-benzimidazol-6-yl)-4-{[4-(1-methylethyl)-4H-thieno[3,2-b]pyrrol-5-yl]carbonyl}-2,3,4,5-tetrahydro-1,4-benzoxazepine. ¹H NMR (400 MHz, DMSO-d₆): δ 12.22 (s, 1H), 7.78-7.18 (m, 7H), 7.11-6.43 (m, 2), 4.86 (s, 2H), 4.56 (m, 1H), 4.24 (s, 2H), 4.04 (m, 2H), 1.52-1.27 (m, 6H); MS (EI) for C₂₇H₂₆N₄O₂S: 471.2 (MH+).

Methyl 5-{[7-(2-methyl-1H-benzimidazol-6-yl)-2,3-dihydro-1,4-benzoxazepin-4(5H)-yl]carbonyl}thiophene-2-carboxylate. ¹H NMR (400 MHz, DMSO-d₆): δ 12.24 (s, 1H), 7.86-7.23 (m, 7H), 7.04 (m, 1H), 4.86 (s, 2H), 4.28 (s, 2H), 4.04 (s, 2H), 3.84 (s, 3H); MS (EI) for C₂₄H₂ᵢN₃O₄S: 448.2 (MH+).

5-{[7-(2-Methyl-1H-benzimidazol-6-yl)-2,3-dihydro-1,4-benzoxazepin-4(5H)-yl]carbonyl}thiophene-2-carboxylic acid. ¹H NMR (400 MHz, DMSO-d₆): δ 7.75-7.27 (m, 7H), 7.05 (m, 1H), 4.86 (s, 2H), 4.29 (s, 2H), 4.05 (s, 2H); MS (EI) for C₂₃H₁₉N₃O₄S: 434.1 (MH+).

Methyl 1-methyl-5-{[7-(2-methyl-1H-benzimidazol-6-yl)-2,3-dihydro-1,4-benzoxazepin-4(5H)-yl]carbonyl}-1H-pyrrole-2-carboxylate. ¹H NMR (400 MHz, CDCl₃); δ 8.09-7.64 (m, 5H), 7.53-7.17 (m, 2H), 6.70-6.49 (m, 1H), 5.30-4.96 (m, 2H), 4.64-3.97 (m, 10H), 2.66 (s, 3H); MS (EI) for C₂₅H₂₄N₄O₄: 445.2 (MH+).

2,2,2-Trifluoro-1-(4-{[7-(1H-indol-3-yl)-2,3-dihydro-1,4-benzoxazepin-4(5H)-yl]carbonyl}phenyl)ethanone. ¹H NMR (400 MHz, DMSO-d₆): δ 11.32 (d, 1H), 8.18-6.93 (m, 10H), 4.84 (s, 1H), 4.43 (s, 1H), 4.28 (s, 1H), 4.18 (s, 1H), 4.09 (s, 1H), 3.72 (s, 3H). MS (EI) for C₂₆H₁₉F₃N₂O₃, found 465 (MH+).

1-(4-{[7-(1H-Pyrazol-4-yl)-2,3-dihydro-1,4-benzoxazepin-4(5H)-yl]carbonyl}phenyl)ethanone. ¹H NMR (400 MHz, CDCl₃): δ 8.02 (d, 1H), 7.98 (d, 1H), 7.86 (s, 1H), 7.69 (s, 1H), 7.59 (s, 0.5H), 7.45 (d, 2H), 7.42-7.35 (m, 1H), 7.08 (dd, 1H), 6.67 (s, 0.5H), 4.85 (s, 1H), 4.43 (s, 1H), 4.25 (br s, 1H), 4.17 (br s, 1H), 4.06-4.01 (m, 1H), 3.81-3.75 (m, 1H), 2.64 (d, 3H). MS (EI) for C₂₁H₁₉N₃O₃, found 362 (MH+).

4-{[2-Chloro-4-(methylsulfonyl)phenyl]carbonyl}-7-(1H-indazol-6-yl)-2,3,4,5-tetrahydro-1,4-benzoxazepine. ¹H NMR (400 MHz, Methanol-d₄): δ 8.17-7.94 (m, 3H), 7.85-7.72 (m, 2H), 7.61-7.42 (m, 3H), 7.21-7.10 (m, 1H), 6.85 (d, 1H), 4.94 (dd, 1H), 4.48 (d, 1H), 4.32-4.03 (m, 3H), 3.76-3.62 (m, 1H), 3.17 (d, 3H). MS (EI) for C₂₄H₂₀ClN₃O₄S, found 482 (MH+).

4-{[4-(Methylsulfonyl)phenyl]carbonyl}-7-(1H-pyrazol-4-yl)-2,3,4,5-tetrahydro-1,4-benzoxazepine. ¹H NMR (400 MHz, CDCl₃): δ 8.01 (t, 2H), 7.86 (s, 1H), 7.69 (s, 1H), 7.61-7.51 (m, 2H), 7.40 (d, 1H), 7.09 (dd, 1H), 6.59 (s, 1H), 4.86 (s, 1H), 4.41 (s, 1H), 4.25 (br s, 1H), 4.18 (br s, 1H), 4.05 (br s, 1H), 3.77 (br s, 1H), 3.09 (d, 3H). MS (EI) for C₂₀H₁₉N₃O₄S, found 398 (MH+).

4-{[2-Chloro-4-(methylsulfonyl)phenyl]carbonyl}-7-(1H-pyrazol-4-yl)-2,3,4,5-tetrahydro-1,4-benzoxazepine. ¹H NMR (400 MHz, CDCl₃): δ 8.06 (d, 0.5H), 8.00 (d, 0.5H), 7.90-7.85 (m, 2H), 7.66 (br s, 1H), 7.58 (d, 0.5H), 7.47 (d, 0.5H), 7.40 (td, 1H), 7.32 (d, 0.5H), 7.08 (t, 1H), 6.41 (d, 0.5H), 5.01 (d, 0.5H), 4.78 (d, 0.5H), 4.49-4.39 (m, 1H), 4.38-4.30 (m, 0.5H), 4.25-4.14 (m, 1H), 4.14-4.07 (m, 0.5H), 4.03-3.90 (m, 1H), 3.72-3.54 (m, 1H), 3.10 (d, 3H). MS (EI) for C₂₀H₁₈ClN₃O₄S, found 432 (MH+) Cl isotope pattern.

7-(1H-Indazol-6-yl)-4-{[2-methyl-4-(methylsulfonyl)-phenyl]carbonyl}-2,3,4,5-tetrahydro-1,4-benzoxazepine. ¹H NMR (400 MHz, Methanol-d₄): δ 8.04 (d, 1H), 7.94-7.72 (m, 4H), 7.47-7.37 (m, 2H), 7.19 (dd, 1H), 7.12 (dd, 1H), 6.51 (d, 1H), 5.03 (br d, 0.5H), 4.49-4.02 (m, 5H), 3.74-3.58 (m, 0.5H), 3.14 (s, 2H), 3.12 (s, 1H), 2.32 (s, 1H), 2.01 (s, 2H). MS (EI) for C₂₅H₂₃N₃O₄S, found 462 (MH+).

4-[(4-Fluoro-2-methylphenyl)carbonyl]-7-(1H-indazol-6-yl)-2,3,4,5-tetrahydro-1,4-benzoxazepine. ¹H NMR (400 MHz, Methanol-d₄): δ 8.06-8.04 (m, 1H), 7.84-7.78 (m, 1H), 7.74-7.72 (m, 1H), 7.59-7.53 (m, 1.5H), 7.44 (dd, 0.5H), 7.24-6.95 (m, 4.5H), 6.71 (d, 0.5H), 5.04-4.83 (m, 1H), 4.56-4.41 (m, 1H), 4.36-4.02 (m, 3H), 3.78-3.61 (m, 1H), 2.23 (s, 1H), 1.98 (s, 2H). MS (EI) for C₂₄H₂₀FN₃O₂, found 402 (MH+).

4-({7-[6-(Methyloxy)pyridin-3-yl]-2,3-dihydro-1,4-benzoxazepin-4(5H)-yl}carbonyl)benzenesulfonamide. ¹H NMR (400 MHz, DMSO-d₆): δ 8.54-8.40 (m, 1H), 8.08-7.93 (m, 2H), 7.85-7.50 (m, 5H), 7.14 (m, 1H), 6.97 (m, 1H), 4.93 (br.s, 1H), 4.61 (br.s, 1H), 4.37 (br.s, 1H), 4.23 (br.s, 1H), 4.09 (br.m, 1H), 3.95 (m, 2H), 3.78 (m, 1H), 1.32 (m, 3H). MS (EI) for C₂₂H₂ᵢN₃O₅S, found 440 (MH+).

(7-(6-Methoxypyridin-3-yl)-2,3-dihydrobenzo[f][1,4]oxazepin-4(5H)-yl)(4-(methylsulfonyl)phenyl)methanone. ¹H NMR (400 MHz, DMSO-d₆): δ 8.47-8.30 (m, 1H), 7.99-7.80 (m, 3H), 7.66 (m, 2H), 7.50 (m, 2H), 7.08 (m, 1H), 6.91-6.86 (m, 1H), 4.87 (s, 1H), 4.52 (s, 1H), 4.29 (br.m, 1H), 4.16 (br.m, 1H), 4.04 (br.m, 1H), 3.90 (m, 3H), 3.71 (m, 1H), 3.27 (s, 3H). MS (EI) for C₂₃H₂₂N₂O₅S, found 439 (MH+).

4-{[2-Bromo-4-(methylsulfonyl)phenyl]carbonyl}-7-(1H-indazol-6-yl)-2,3,4,5-tetrahydro-1,4-benzoxazepine. ¹H NMR (400 MHz, Methanol-d₄): δ 8.32-8.21 (m, 1H), 8.08-7.98 (m, 2H), 7.85-7.71 (m, 2H), 7.61-7.41 (m, 3H), 7.22-7.09 (m, 1.5H), 6.59 (d, 0.5H), 5.00 (d, 0.5H), 4.88 (d, 0.5H), 4.50 (d, 0.5H), 4.39 (d, 0.5H), 4.32-4.15 (m, 3H), 4.09-4.02 (m, 0.5H), 3.75-3.61 (m, 0.5H), 3.17 (d, 3H). MS (EI) for C₂₄H₂₀BrN₃O₄S, found 526, 528 (Br isotopes, MH+).

7-[5-(Methyloxy)pyridine-2-yl]-4-{[4-(methylsulfonyl)phenyl]carbonyl}-2,3,4,5-tetrahydro-1,4-benzoxazepine. ¹H NMR (400 MHz, DMSO-d₆): δ 8.37-8.29 (m, 1H), 8.05-7.86 (m, 4H), 7.67 (m, 2H), 7.67 (m, 1H), 7.49-7.39 (m, 1H), 7.24-7.05 (m, 1H), 4.88 (s, 2H), 4.16 (br.m, 2H), 3.87 (m, 3H), 3.72 (br.m, 2H), 3.26 (s, 3H). MS (EI) for C₂₃H₂₂N₄O₅S, found 439 (MH+).

2,2,2-Trifluoro-1-[4-({7-[6-(methyloxy)pyridin-3-yl]-2,3-dihydro-1,4-benzoxazepin-4(5H)-yl}carbonyl)phenyl]ethanone. ¹H NMR (400 MHz, DMSO-d₆): δ 8.48-8.31 (m, 1H), 8.09-8.01 (m, 1H), 7.80-7.66 (m, 3H), 7.59-7.28 (m, 3H), 7.07 (m, 1H), 6.92-6.69 (m, 1H), 4.85 (m, 1H), 4.49 (m, 1H), 4.27-4.16 (m, 1H), 4.02 (m, 1H), 3.89 (m, 3H). MS (EI) for C₂₄H₁₉F₃N₂O₄, found 475 (MH+).

4-{[2-Ethyl-4-(methylsulfonyl)phenyl]carbonyl}-7-(1H-indazol-6-yl)-2,3,4,5-tetrahydro-1,4-benzoxazepine. ¹H NMR (400 MHz, CDCl₃): δ 8.05 (d, 2H), 8.00 (d, 1H), 7.75 (d, 1H), 7.54 (dd, 1H), 7.33-7.26 (m, 3H), 7.16 (d, 1H), 6.34 (d, 1H), 4.38 (d, 1H), 4.29-4.22 (m, 5H), 3.21 (s, 3H), 2.55-2.44 (m, 1H), 2.32-2.20 (m, 1H), 1.14 (t, 3H). MS (EI) for C₂₆H₂₅N₃O₄S, found 476 (MH+).

7-(1H-Indazol-6-yl)-44 {4-[(trifluoromethyl)sulfonyl]phenyl}-carbonyl)-2,3,4,5-tetrahydro-1,4-benzoxazepine. ¹H NMR (400 MHz, DMSO-d₆): δ 13.15 (d, 1H), 8.21 (d, 2H), 8.12-8.07 (m, 1H), 7.88-7.82 (m, 2H), 7.79-7.66 (m, 2H), 7.63-7.56 (m, 1.5H), 7.43 (d, 0.5H), 7.16-7.08 (m, 1.5H), 6.86 (s, 0.5H), 4.92 (br s, 1H), 4.52 (br s, 1H), 4.31 (br s, 1H), 4.18 (br s, 1H), 4.07 (br s, 1H), 3.71 (br s, 1H). MS (EI) for C₂₄H₁₈F₃N₃O₄S, found 502 (MH+).

7-(1H-Indazol-6-yl)-4-({4-[(methylsulfonyl)methyl]-phenyl}carbonyl)-2,3,4,5-tetrahydro-1,4-benzoxazepine. ¹H NMR (400 MHz, DMSO-d₆): δ 13.13 (d, 1H), 8.09 (s, 1H), 7.86-7.79 (m, 1H), 7.73 (br d, 1H), 7.61-7.40 (m, 5H), 7.34-7.21 (m, 1.5H), 7.14-7.06 (m, 1H), 6.91 (br s, 0.5H), 4.87 (br s, 1H), 4.64-4.51 (m, 3H), 4.33-3.99 (m, 3H), 3.78 (br s, 1H), 2.97-2.86 (m, 3H). MS (EI) for C₂₅H₂₃N₃O₄S, found 462 (MH+).

2,2,2-Trifluoro-1-(4-{[7-(2-methyl-1H-benzimidazol-6-yl)-2,3-dihydro-1,4-benzoxazepin-4(5H)-yl]carbonyl}phenyl)ethanone. ¹H NMR (400 MHz, DMSO-d₆): δ 8.15-6.55 (m, 10H), 4.84 (m, 1H), 4.51 (s, 1H), 4.32-4.04 (m, 3H), 3.73 (s, 1H), 2.48 (s, 3H). MS (EI) for C₂₆H₂₀F₃N₃O₃, found 480 (MH+).

2,2,2-Trifluoro-1-[4-({7-[5-(methyloxy)pyridin-3-yl]-2,3-dihydro-1,4-benzoxazepin-4(5H)-yl}carbonyl)phenyl]ethanone. ¹H NMR (400 MHz, DMSO-d₆): δ 8.49 (s, 1H), 8.31-8.09 (m, 2H), 7.81-7.62 (m, 4H), 7.52-7.31 (m, 1H), 7.14-6.89 (m, 1H), 6.56 (s, 1H), 4.90 (s, 1H), 4.53 (br.m, 1H), 4.31-4.20 (m, 2H), 4.04-3.92 (m, 3H), 3.73 (m, 2H). MS (EI) for C₂₄H₁₉N₃O₄, found 475 (MH+).

5-(4-{[4-(Methylsulfonyl)phenyl]carbonyl}-2,3,4,5-tetrahydro-1,4-benzoxazepin-7-yl)pyridin-2-amine. ¹H NMR (400 MHz, DMSO-d₆): δ 8.23 (m, 1H), 7.99 (m, 1H), 7.67-7.41 (m, 4H), 7.03 (m, 1H), 6.72-6.47 (m, 1H), 6.05 (s, 2H), 4.84 (s, 1H), 4.47 (s, 1H), 4.24 (m, 1H), 4.12 (m, 1H), 4.03 (m, 1H), 3.70 (m, 2H), 3.27 (m, 2H). MS (EI) for C₂₂H₂₁N₃O₄S, found 424 (MH+).

4-{[7-(6-Aminopyridin-3-yl)-2,3-dihydro-1,4-benzoxazepin-4(5H)-yl]carbonyl}benzenesulfonamide. ¹H NMR (400 MHz, DMSO-d₆): δ 8.23-8.07 (m, 1H), 7.87-7.78 (m, 3H), 7.58-7.43 (m, 5H), 7.04 (m, 1H), 6.75-6.57 (m, 1H), 4.83 (br.s, 1H), 4.50 (br.s, 1H), 4.26 (br.m, 1H), 4.13 (br.m, 1H), 4.02 (br.m, 1H), 3.71 (br.m, 2H), 3.27 (m, 2H). MS (EI) for C₂₁H₂₀N₄O₄S, found 425 (MH+).

(7-(1H-Benzo[d]imidazol-6-yl)-2,3-dihydrobenzo[f][1,4]oxazepin-4(5H)-yl)(4-(4-methylpiperazin-1-ylsulfonyl)phenyl)-methanone. ¹H NMR (400 MHz, DMSO-d₆): δ 9.60-9.49 (m, 1H), 8.04 (s, 1H), 7.96-7.75 (m, 5H), 7.71-7.06 (m, 5H), 4.92 (s, 1H), 4.55 (s, 1H), 4.35 (m, 1H), 4.18 (m, 1H), 4.05 (m, 1H), 3.84-3.67 (m, 5H), 3.25-3.06 (m, 3H), 2.78-2.66 (m, 4H). MS (EI) for C₂₈H₂₉N₅O₄S, found 532 (MH+).

4-{[4-(Ethylsulfonyl)-2-methylphenyl]carbonyl}-7-(2-methyl-1H-benzimidazol-6-yl)-2,3,4,5-tetrahydro-1,4-benzoxazepine. ¹H NMR (400 MHz, DMSO-d₆): δ 8.00-6.58 (m, 9H), 5.04-4.80 (m, 1H), 4.54-3.94 (m, 5H), 3.53 (br s, 1H), 3.36-3.19 (m, 2H), 2.89-2.75 (m, 3H), 2.25-1.70 (m, 3H), 1.28-0.93 (m, 3H). MS (EI) for C₂₇H₂₇N₃O₄S, found 488 (MH−).

4-{[2-Ethyl-4-(methylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 7.96-7.69 (m, 4H), 7.63-7.53 (m, 1H), 7.45-7.37 (m, 1H), 7.30-7.24 (m, 1H), 7.18-6.64 (m, 2H), 5.06-4.81 (m, 1H), 4.48-4.01 (m, 5H), 3.58-3.50 (m, 1H), 3.26-3.20 (m, 3H), 2.83-2.77 (m, 3H), 2.66-2.51 (m, 1H), 2.41-2.20 (m, 1H), 1.16-0.95 (m, 3H). MS (EI) for C₂₇H₂₇N₃O₄S, found 488 (MH−).

7-{2-[2-(Methyloxy)ethyl]-1H-benzimidazol-6-yl}-4-{[4-(methylsulfonyl)phenyl]carbonyl}-2,3,4,5-tetrahydro-1,4-benzoxazepine. ¹H NMR (400 MHz, DMSO-d₆): δ 12.28 (m, 1H), 7.99 (m, 2H), 7.68 (m, 3H), 7.52 (m, 2H), 7.43 (m, 1H), 7.08 (m, 1H), 6.82 (m, 1H), 4.88 (s, 1H), 4.53 (s, 1H), 4.27 (m, 1H), 4.15 (m, 1H), 4.05 (m, 1H), 3.75 (m, 3H), 3.28 (m, 6H), 3.07 (m, 2H). MS (EI) for C₂₇H₂₇N₃O₅S, found 506 (MH+).

7-(2-Methyl-1H-benzimidazol-6-yl)-4-[(1-methyl-1H-indol-3-yl)carbonyl]-2,3,4,5-tetrahydro-1,4-benzoxazepine. ¹H NMR (400 MHz, DMSO-d₆): δ 7.70 (m, 2H), 7.62 (s, 1H), 7.52-7.48 (m, 4H), 7.34 (m, 1H), 7.22 (m, 1H), 7.12 (m, 1H), 7.04 (m, 1H), 4.86 (s, 2H), 4.23 (s, 2H), 4.07 (s, 2H), 3.82 (s, 3H), 1.71 (s, 3H). MS (EI) for C₂₇H₂₄N₄O₂, found 437 (MH+).

2,2,2-Trifluoro-1-(4-{[7-(2-methyl-1H-benzimidazol-6-yl)-2,3-dihydro-1,4-benzoxazepin-4(5H)-yl]carbonyl}phenyl)ethanol. ¹H NMR (400 MHz, DMSO-d₆): δ 7.76-6.76 (m, 10H), 5.29 (m, 1H), 4.87 (s, 1H), 4.56 (s, 1H), 4.28 (s, 1H), 4.18 (s, 1H), 4.00 (s, 1H), 3.73 (s, 1H), 2.53 (s, 3H). MS (EI) for C₂₆H₂₂F₃N₃O₃, found 482 (MH+).

4-[(4-Iodophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.98-7.54 (m, 7H), 7.25-6.99 (m, 3H), 4.91-4.52 (m, 2H), 4.36-4.12 (m, 2H), 4.04-3.70 (m, 2H), 2.79 (m, 3H). MS (EI) for C₂₄H₂₀IN₃O₂, found 508 (MH−).

7-(2-Methyl-1H-benzimidazol-6-yl)-4-[(1-methyl-1H-indol-2-yl)carbonyl]-2,3,4,5-tetrahydro-1,4-benzoxazepine. ¹H NMR (400 MHz, DMSO-d₆): δ 11.9 (bs, 1H), 7.72 (m, 1H), 7.63-7.50 (m, 6H), 7.23 (m, 1H), 7.09-7.06 (m, 2H), 6.71-6.43 (m, 1H), 4.87 (m, 2H), 4.22 (m, 2H), 4.03 (s, 2H), 3.54 (d, 3H), 2.56 (s, 3H). MS (EI) for C₂₇H₂₄N₄O₂, found 437 (MH+).

2-Chloro-N,N-dimethyl-4-(7-(2-methyl-1H-benzo[c/]imidazol-6-yl)-2,3,4,5-tetrahydrobenzo[f][1,4]oxazepine-4-carbonyl)benzenesulfonamide. ¹H NMR (400 MHz, DMSO-d₆): δ 7.99-7.94 (m, 1H), 7.78-7.40 (m, 6H), 7.28-6.95 (m, 2H), 4.86 9s, 1H), 4.53 (s, 1H), 4.29 (m, 1H), 4.16 (m, 1H), 4.03 (m, 1H), 3.74 (m, 1H), 2.84 (s, 3H), 2.76 (s, 3H). MS (EI) for C₂₆H₂₅ClN₄O₄S, found 525, 527 (M, M+2).

7-(2-Methyl-1H-benzimidazol-6-yl)-4-{[2-methyl-4-(methylsulfonyl)phenyl]carbonyl}-2,3,4,5-tetrahydro-1,4-benzoxazepine hydrochloride. ¹H NMR (400 MHz, DMSO-d₆): δ 8.05-6.63 (m, 9H), 5.11-4.79 (m, 1H), 4.51-3.97 (m, 4H), 3.55 (br s, 1H), 3.28-3.14 (m, 3H), 2.88-2.71 (m, 3H), 2.29-1.89 (m, 3H). MS (EI) for C₂₇H₂₇N₃O₄S, found 488 (MH−).

4-{[4-(1,1-Dimethylethyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 7.95-7.07 (m, 10H), 4.91-4.52 (m, 2H), 4.30-4.13 (m, 2H), 4.03-3.76 (m, 2H), 2.76 (br s, 3H), 1.29 (s, 9H). MS (EI) for C₂₈H₂₉N₃O₂, found 440 (MH+).

4-[(4-Bromophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.98-7.72 (m, 4H), 7.68-7.57 (m, 3H), 7.39-7.04 (m, 3H), 4.90-4.56 (m, 2H), 4.35-4.14 (m, 2H), 4.04-3.73 (m, 2H), 2.80 (br s, 3H). MS (EI) for C₂₄H₂₀BrN₃O₂, found 464, 466 (M, M+2).

4-[(4-Chlorophenyl)carbonyl]-7-(2-methyl-1H-benzimidazol-6-yl)-2,3,4,5-tetrahydro-1,4-benzoxazepine. ¹H NMR (400 MHz, DMSO-d₆): δ 7.99-7.71 (m, 3H), 7.67-7.23 (m, 6H), 7.17-7.02 (m, 1H), 4.95-4.54 (m, 2H), 4.38-4.10 (m, 2H), 4.07-3.70 (m, 2H), 2.83 (s, 3H). MS (EI) for C₂₄H₂₀ClN₃O₂, found 418 (MH+).

4-[(4-Bromo-1-ethyl-3-methyl-1H-pyrazol-5-yl)carbonyl]-7-(2-methyl-1H-benzimidazol-6-yl)-2,3,4,5-tetrahydro-1,4-benzoxazepine. ¹H NMR (400 MHz, DMSO-d₆): δ 12.25 (s, 1H), 7.67 (d, 1H), 7.59-7.34 (m, 3H), 7.27-6.75 (m, 2H), 4.9 (m, 1H), 4.57 (m, 1H), 4.41-3.52 (m, 6H), 2.26-2.11 (m, 3H), 1.27-0.94 (m, 3H). MS (EI) for C₂₄H₂₄BrN₅O₂, found 494,496 (M, M+2).

7-(2-Methyl-1H-benzimidazol-6-yl)-4-(phenylcarbonyl)-2,3,4,5-tetrahydro-1,4-benzoxazepine. ¹H NMR (400 MHz, DMSO-d₆): δ 7.76-7.22 (m, 10H), 7.11-7.03 (m, 1H), 4.90-4.49 (m, 2H), 4.30-3.73 (m, 4H), 2.56 (br s, 3H). MS (EI) for C₂₄H₂₁N₃O₂, found 384 (MH+).

1-(4-{[7-(2-Methyl-1H-benzimidazol-6-yl)-2,3-dihydro-1,4-benzoxazepin-4(5H)-yl]carbonyl}phenyl)ethanone. ¹H NMR (400 MHz, DMSO-d₆): δ 8.06-6.92 (m, 10H), 4.97-4.51 (m, 2H), 4.40-3.69 (m, 4H), 2.83 (s, 3H), 2.56 (br s, 3H). MS (EI) for C₂₆H₂₃N₃O₃, found 426 (MH+).

4-(1H-Indol-5-ylcarbonyl)-7-(2-methyl-1H-benzimidazol-6-yl)-2,3,4,5-tetrahydro-1,4-benzoxazepine. ¹H NMR (400 MHz, DMSO-d₆): δ 11.40 (s, 1H), 8.03-6.37 (m, 11H), 4.99-3.82 (m, 6H), 2.82 (s, 3H). MS (EI) for C₂₆H₂₂N₄O₂, found 423 (MH+).

N-Cyclopentyl-4-{[7-(2-methyl-1H-benzimidazol-6-yl)-2,3-dihydro-1,4-benzoxazepin-4(5H)-yl]carbonyl}-3-(trifluoromethyl)benzenesulfonamide. ¹H NMR (400 MHz, DMSO-d₆): δ 12.20 (br, 1H), 8.30 (m, 2H), 7.90 (s, 1H), 7.80 (m, 1H), 7.50 (m, 2H), 7.10 (m, 2H), 6.60 (s, 1H), 4.98 (d, 1H), 4.40 (m, 2H), 4.20 (m, 3H), 3.52 (m, 1H), 3.01 (m, 1H), 2.51 (s, 3H), 1.25 (m, 4H), 0.60 (m, 4H). MS (EI) for C₃₀H₂₉F₃N₄O₄S, found 599 (MH+).

3-Methyl-4-{[7-(2-methyl-1H-benzimidazol-6-yl)-2,3-dihydro-1,4-benzoxazepin-4(5H)-yl]carbonyl}-N-(1-methylethyl)benzenesulfonamide. ¹H NMR (400 MHz, DMSO-d₆): δ 12.20 (br, 1H), 7.98 (s, 1H), 6.50 (s, 1H), 7.10 (m, 7H), 6.50 (s, 1H), 4.98 (m, 1H), 4.48 (s, 2H), 4.25 (m, 2H), 3.52 (m, 2H), 2.47 (s, 3H), 1.98 (s, 3H), 1.01 (d, 3H), 0.98 (d, 3H). MS (EI) for C₂₈H₃₀N₄O₄S, found 519 (MH+).

N-Cyclopropyl-3-methyl-4-{[7-(2-methyl-1H-benzimidazol-6-yl)-2,3-dihydro-1,4-benzoxazepin-4(5H)-yl]carbonyl}benzenesulfonamide. ¹H NMR (400 MHz, DMSO-d₆): δ 12.20 (br, 1H), 7.98 (s, 1H), 6.50 (s, 1H), 7.10 (m, 7H), 6.50 (s, 1H), 4.98 (m, 1H), 4.48 (s, 2H), 4.25 (m, 2H), 3.52 (m, 2H), 2.47 (s, 3H), 1.98 (s, 3H), 0.52 (m, 2H), 0.53 (m, 2H). MS (EI) for C₂₈H₂₈N₄O₄S, found 517 (MH+).

4-{[2-Ethyl-4-(methylsulfonyl)phenyl]carbonyl}-7-[2-(fluoromethyl)-1H-benzimidazol-6-yl]-2,3,4,5-tetrahydro-1,4-benzoxazepine hydrochloride salt. ¹H NMR (400 MHz, DMSO-d₆): δ 7.98 (br s, 0.5H), 7.89-7.83 (m, 2H), 7.83-7.76 (m, 3H), 7.74 (d, 0.5H), 7.63 (dd, 0.5H), 7.58 (dd, 0.5H), 7.44 (d, 0.5H), 7.42 (dd, 0.5H), 7.31 (d, 0.5H), 7.12 (dd, 1H), 6.67 (d, 0.5H), 5.93 (d, 1H), 5.81 (d, 1H), 5.05 (d, 0.5H), 4.87 (d, 0.5H), 4.50-3.50 (m, 5H), 3.25 (d, 3H), 2.69-2.51 (m, 1H), 2.43-2.22 (m, 1H), 1.15 (t, 1.5H), 1.01 (t, 1.5H). MS (EI) for C₂₇H₂₆FN₃O₄S, found 508 (MH+).

4-{[2-Ethyl-5-fluoro-4-(methylsulfonyl)phenyl]carbonyl}-7-(2-methyl-1H-benzimidazol-6-yl)-2,3,4,5-tetrahydro-1,4-benzoxazepine. ¹H NMR (400 MHz, DMSO-d₆): δ 12.25 (br.s, 1H), 7.75-7.67 (m, 2H), 7.52-7.41 (m, 3H), 7.25-7.07 (m, 2H), 6.80 (s, 1H), 5.00-4.81 (m, 1H), 4.50-4.37 (m, 1H), 4.26-4.07 (m, 3H), 3.58 (m, 1H), 3.61 (m, 3H), 2.57 (m, 1H), 2.49 (m, 3H), 2.31 (m, 1H), 1.11-0.99 (tt, 3H). MS (EI) for C₂₇H₂₆FN₃O₄S, found 508 (MH+).

N-(1,1-dimethylethyl)-3-methyl-4-{[7-(2-methyl-1H-benzimidazol-6-yl)-2,3-dihydro-1,4-benzoxazepin-4(5H)-yl]carbonyl}benzenesulfonamide. ¹H NMR (400 MHz, DMSO-d₆): δ 12.2 (br, 1H), 7.98 (s, 1H), 6.50 (s, 1H), 7.10 (m, 7H), 6.50 (s, 1H), 4.98 (m, 1H), 4.48 (s, 2H), 4.25 (m, 2H), 3.52 (m, 1H), 2.47 (s, 3H), 1.98 (s, 3H), 1.00 (s, 9H). MS (EI) for C₂₀H₃₂N₄O₄S, found 533 (MH+).

[7-(2-Methyl-1H-benzimidazol-6-yl)-2,3-dihydro-1,4-benzoxazepin-4(5H)-yl](1-methyl-5-phenyl-1H-pyrrol-2-yl)methanone. ¹H NMR (400 MHz, DMSO-d₆): δ 12.10 (br, 1H), 7.51 (m, 10H), 7.01 (d, 1H), 6.40 (s, 1H), 6.22 (s, 1H), 4.83 (s, 3H), 4.30 (brs, 2H), 4.0 (brs, 2H), 2.50 (s, 3H). MS (EI) for C₂₉H₂₆N₄O₂, found 463 (MH+).

4-{[2-Ethyl-4-(methylsulfonyl)phenyl]carbonyl}-7-(1-methyl-1H-benzimidazol-5-yl)-2,3,4,5-tetrahydro-1,4-benzoxazepine hydrochloride salt. ¹H NMR (400 MHz, DMSO-d₆): δ 9.57 (d, 1H), 8.08-8.05 (m, 1H), 7.99-7.92 (m, 1H), 7.88 (dd, 1H), 7.84-7.76 (m, 2H), 7.68-7.58 (m, 1.5H), 7.43 (d, 0.5H), 7.30 (d, 0.5H), 7.13 (dd, 1H), 6.76 (d, 0.5H), 5.05 (d, 0.5H), 4.88 (d, 0.5H), 4.53-4.38 (m, 1H), 4.38-4.13 (m, 2H), 4.13-4.05 (m, 1H), 4.08 (d, 3H), 3.61-3.52 (m, 1H), 3.24 (d, 3H), 2.69-2.51 (m, 1H), 2.48-2.20 (m, 1H), 1.14 (t, 1.5H), 1.00 (t, 1.5H). MS (EI) for C₂₇H₂₇N₃O₄S, found 490 (MH+).

(4-Ethyl-4H-pyrrolo[2,3-d][1,3]thiazol-5-yl)[7-(2-methyl-1H-benzimidazol-6-yl)-2,3-dihydro-1,4-benzoxazepin-4(5H)-yl]methanone. ¹H NMR (400 MHz, CDCl₃): δ 8.62 (s, 1H), 7.56-7.52 (m, 2H), 7.47-7.42 (dd, 1H), 7.37-7.27 (m, 1H), 7.14-7.09 (d, 2H), 6.55 (s, 1H), 4.88 (s, 2H), 4.39 (q, 2H), 4.30-4.16 (m, 4H), 2.66 (s, 3H), 1.34 (t, 3H). MS (EI) for C₂₅H₂₃N₅O₂S, found 458 (MH+).

7-(1,3-Benzothiazol-5-yl)-4-{[2-ethyl-4-(methylsulfonyl)-phenyl]carbonyl}-2,3,4,5-tetrahydro-1,4-benzoxazepine. ¹H NMR (400 MHz, DMSO-d₆): δ 9.44 (d, 1H), 8.37 (d, 0.5H), 8.26 (d, 0.5H), 8.19-8.13 (m, 1H), 7.89-7.77 (m, 3H), 7.70-7.63 (m, 1H), 7.53 (dd, 0.5H), 7.45 (d, 0.5H), 7.31 (d, 0.5H), 7.14-7.09 (m, 1H), 6.79 (d, 0.5H), 5.09-4.83 (m, 1H), 4.58-4.04 (m, 4H), 3.63-3.50 (m, 1H), 2.68-2.23 (m, 2H), 1.15 (t, 1.5H), 1.01 (t, 1.5H). MS (EI) for C₂₆H₂₄N₂O₄S₂, found 493 (MH+).

7-(1-Ethyl-1H-benzimidazol-5-yl)-4-{[2-ethyl-4-(methylsulfonyl)phenyl]carbonyl}-2,3,4,5-tetrahydro-1,4-benzoxazepine. ¹H NMR (400 MHz, DMSO-d₆): δ 9.71-9.63 (m, 1H), 8.16-7.75 (m, 5H), 7.69-7.59 (m, 1.5H), 7.44 (d, 0.5H), 7.30 (d, 0.5H), 7.14 (dd, 1H), 6.77 (d, 0.5H), 5.10-4.84 (m, 1H), 4.59-4.08 (m, 6H), 3.62-3.53 (m, 1H), 3.26 (s, 1.5H), 3.22 (s, 1.5H), 2.69-2.50 (m, 2H), 1.55 (q, 3H), 1.15 (t, 1.5H), 1.01 (t, 1.5H). MS (EI) for C₂₈H₂₉N₃O₄S, found 504 (MH+).

4-{[2-Ethyl-4-(methylsulfonyl)phenyl]carbonyl}-7-(2-methyl-1,3-benzoxazol-5-yl)-2,3,4,5-tetrahydro-1,4-benzoxazepine. ¹H NMR (400 MHz, DMSO-d₆): δ 7.92 (d, 0.5H), 7.86 (dd, 1H), 7.83-7.77 (m, 1H), 7.75-7.72 (m, 1H), 7.69 (d, 0.5H), 7.67-7.61 (m, 1H), 7.58 (dd, 0.5H), 7.55 (dd, 0.5H), 7.44 (d, 0.5H), 7.35 (dd, 0.5H), 7.30 (d, 0.5H), 7.09 (dd, 1H), 6.68 (d, 0.5H), 5.04 (d, 0.5H), 4.85 (d, 0.5H), 4.50-4.35 (m, 1H), 4.35-4.02 (m, 3H), 3.65-3.49 (m, 1H), 3.25 (d, 3H), 2.71-2.51 (m, 1H), 2.63 (d, 3H), 2.43-2.20 (m, 1H), 1.15 (t, 1.5H), 1.01 (t, 1.5H). MS (EI) for C₂₇H₂₆N₂O₅S, found 491 (MH+).

7-(1-Methyl-1H-benzimidazol-5-yl)-4-({4-[(3-morpholin-4-ylpropyl)sulfonyl]phenyl}carbonyl)-2,3,4,5-tetrahydro-1,4-benzoxazepine acetate salt. ¹H NMR (400 MHz, DMSO-d₆): δ 8.24-8.20 (m, 1H), 7.96 (t, 2H), 7.90 (s, 0.5H), 7.75-7.70 (m, 1H), 7.70-7.51 (m, 5H), 7.17 (m, 1.5H), 4.89 (br s, 1H), 4.52 (br s, 1H), 4.29 (s, 1H), 4.19-4.13 (m, 1H), 4.08-4.02 (m, 1H), 3.89-3.84 (m, 3H), 3.78-3.68 (m, 1H), 3.57-3.33 (m, 6H), 2.39-2.11 (m, 6H), 1.79-1.62 (m, 2H). MS (EI) for C₃₁H₃₄N₄O₅S, found 575 (MH+).

4-{[2-Ethyl-4-(methylsulfonyl)phenyl]carbonyl}-7-(2-methyl-1,3-benzothiazol-5-yl)-2,3,4,5-tetrahydro-1,4-benzoxazepine. ¹H NMR (400 MHz, DMSO-d₆): δ 8.19 (d, 0.5H), 8.12 (d, 0.5H), 8.03 (d, 0.5H), 7.96 (d, 0.5H), 7.89-7.78 (m, 2.5H), 7.73 (dd, 0.5H), 7.67-7.59 (m, 1H), 7.47-7.41 (m, 1H), 7.32 (d, 0.5H), 7.14-7.09 (m, 1H), 6.74 (d, 0.5H), 5.09-4.82 (m, 1H), 4.52-4.03 (m, 4H), 3.64-3.50 (m, 1H), 3.31-3.23 (m, 3H), 2.86-2.80 (m, 3H), 2.69-2.22 (m, 2H), 1.15 (t, 1.5H), 1.01 (t, 1.5H). MS (EI) for C₂₇H₂₆N₂O₄S₂, found 507 (MH+).

7-(1,2-Dimethyl-1H-benzimidazol-5-yl)-4-{[2-ethyl-4-(methylsulfonyl)phenyl]carbonyl}-2,3,4,5-tetrahydro-1,4-benzoxazepine. ¹H NMR (400 MHz, DMSO-d₆): δ 8.05-7.99 (m, 1.5H), 7.94-7.89 (m, 1H), 7.88 (d, 0.5H), 7.85-7.80 (m, 1.5H), 7.80-7.74 (m, 0.5H), 7.66 (dd, 0.5H), 7.61 (dd, 0.5H), 7.57 (dd, 0.5H), 7.43 (d, 0.5H), 7.29 (d, 0.5H), 7.13 (dd, 1H), 6.77 (d, 0.5H), 5.05 (d, 0.5H), 4.88 (d, 0.5H), 4.54-4.39 (m, 1H), 4.39-4.07 (m, 3H), 3.95 (d, 3H), 3.61-3.52 (m, 1H), 3.24 (d, 3H), 2.84 (d, 3H), 2.69-2.51 (m, 1H), 2.48-2.20 (m, 1H), 1.14 (t, 1.5H), 1.00 (t, 1.5H). MS (EI) for C₂₈H₂₉N₃O₄S, found 504 (MH+).

2-[5-(4-{[2-Ethyl-4-(methylsulfonyl)phenyl]carbonyl}-2,3,4,5-tetrahydro-1,4-benzoxazepin-7-yl)-1H-benzimidazol-1-yl]ethanol. ¹H NMR (400 MHz, DMSO-d₆): δ 8.37-8.23 (m, 1H), 7.92-7.50 (m, 6H), 7.30 (d, 1H), 7.09-7.03 (m, 2H), 5.06-4.76 (m, 2H), 4.47-3.99 (m, 6H), 3.78-3.67 (m, 2H), 3.61-3.47 (m, 1H), 3.23 (s, 3H), 2.66-2.22 (m, 2H), 1.05 (m, 3H). MS (EI) for C₂₈H₂₉N₃O₅S, found 520 (MH+).

1-[5-(4-{[2-Ethyl-4-(methylsulfonyl)phenyl]carbonyl}-2,3,4,5-tetrahydro-1,4-benzoxazepin-7-yl)-1-methyl-1H-benzimidazol-2-yl]-N-methylmethanamine. ¹H NMR (400 MHz, DMSO-d₆): δ 9.60 (br s, 1H), 7.94 (d, 0.5H), 7.87 (d, 0.5H), 7.84-7.71 (m, 3H), 7.68 (dd, 0.5H), 7.65 (d, 0.5H), 7.60 (dd, 0.5H), 7.56 (dd, 0.5H), 7.43 (d, 0.5H), 7.38 (dd, 0.5H), 7.32 (d, 0.5H), 7.09 (dd, 1H), 6.69 (d, 0.5H), 5.04 (d, 0.5H), 4.85 (d, 0.5H), 4.60 (d, 2H), 4.51-4.37 (m, 1H), 4.36-3.98 (m, 3H), 3.87 (d, 3H), 3.62-3.54 (m, 1H), 3.25 (d, 3H), 2.77 (br s, 3H), 2.69-2.51 (m, 1H), 2.48-2.20 (m, 1H), 1.15 (t, 1.5H), 1.00 (t, 1.5H). MS (EI) for C₂₉H₃₂N₄O₄S, found 533 (MH⁺).

2-[5-(4-{[2-ethyl-4-(methylsulfonyl)phenyl]carbonyl}-2,3,4,5-tetrahydro-1,4-benzoxazepin-7-yl)-1H-benzimidazol-1-yl]-N-methylethanamine. ¹H NMR (400 MHz, DMSO-d₆): δ 9.70-9.60 (m, 1H), 9.29 (br s, 2H), 8.20 (d, 0.5H), 8.13-8.05 (m, 1H), 8.00-7.59 (m, 5H), 7.43 (d, 0.5H), 7.30 (d, 0.5H), 7.14 (dd, 1H), 6.82 (d, 0.5H), 5.11-4.83 (m, 3H), 4.55-4.05 (m, 4H), 3.28-3.21 (m, 3H), 2.68-2.54 (m, 4H), 2.47-2.34 (m, 2H), 1.15 (t, 1.5H), 1.01 (t, 1.5H). MS (EI) for C₂₉H₃₂N₄O₄S, found 533 (MH+).

1-[6-(9-Fluoro-4-{[3-fluoro-2-methyl-4-(methylsulfonyl)phenyl]carbonyl}-2,3,4,5-tetrahydro-1,4-benzoxazepin-7-yl)-1H-benzimidazol-2-yl]-N-methylmethanamine. ¹H NMR (400 MHz, DMSO-d₆): δ 7.79-7.70 (m, 1.5H), 7.61-7.44 (m, 3.5H), 7.32 (d, 0.5H), 7.24-7.15 (m, 1H), 6.61 (s, 0.5H), 5.05-4.85 (m, 1H), 4.57-3.98 (m, 4H), 3.90-3.85 (m, 2H), 3.71-3.54 (m, 1H), 3.34 (s, 3H), 2.35-2.31 (m, 3H), 2.14 (s, 1.5H), 1.82 (s, 1.5H). MS (EI) for C₂₇H₂₆F₂N₄O₄S, found 541 (MH+).

6-(4-{[2-Ethyl-3-fluoro-4-(methylsulfonyl)phenyl]carbonyl}-2,3,4,5-tetrahydro-1,4-benzoxazepin-7-yl)-1,3-benzothiazol-2-amine. ¹H NMR (400 MHz, DMSO-d₆): δ 7.99 (d, 0.5H), 7.80-7.72 (m, 1H), 7.70 (dd, 1H), 7.57-7.47 (m, 3H), 7.39 (d, 0.5H), 7.34-7.25 (m, 1H), 7.13 (d, 0.5H), 7.06 (dd, 1H), 6.76 (d, 0.5H), 5.00 (d, 0.5H), 4.81 (d, 0.5H), 4.47-4.35 (m, 1H), 4.34-4.00 (m, 3H), 3.66-3.51 (m, 1H), 3.36 (d, 3H), 2.72-2.57 (m, 0.5H), 2.51-2.31 (m, 1H), 2.16-1.94 (m, 0.5H), 1.10 (t, 1.5H), 1.00 (t, 1.5H). MS (EI) for C₂₆H₂₄FN₃O₄S₂, found 526 (MH+).

N-{[6-(4-{[2-Ethyl-3-fluoro-4-(methylsulfonyl)phenyl]-carbonyl}-2,3,4,5-tetrahydro-1,4-benzoxazepin-7-yl)-1H-benzimidazol-2-yl]methyl}ethanamine. ¹H NMR (400 MHz, DMSO-d₆): δ 12.32 (br.s, 1H), 7.75 (m, 2H), 7.53 (m, 3H), 7.25-6.76 (m, 3H), 5.03-4.81 (m, 1H), 4.43 (m, 2H), 4.17-3.95 (m, 6H), 3.64 (m, 6H), 2.63 (m, 2H), 2.33 (m, 1H), 1.08-0.99 (m, 3H). MS (EI) for C₂₉H₃ᵢFN₄O₄S, found 551 (MH+).

N-{[6-(4-{[2-Ethyl-3-fluoro-4-(methylsulfonyl)phenyl]-carbonyl}-2,3,4,5-tetrahydro-1,4-benzoxazepin-7-yl)-1H-benzimidazol-2-yl]methyl}acetamide. ¹H NMR (400 MHz, DMSO-d₆): δ 8.56 (q, 1H), 7.78-7.64 (m, 2H), 7.57-7.41 (m, 3H), 7.27 (d, 0.5H), 7.19-7.13 (m, 1H), 7.06 (dd, 1H), 6.73 (d, 0.5H), 5.04-4.76 (m, 1H), 4.50-4.27 (m, 3.5H), 4.20-3.94 (m, 2.5H), 3.64-3.51 (m, 1H), 3.33 (s, 3H), 2.70-2.28 (m, 2H), 1.91 (s, 3H), 1.08 (t, 1.5H), 0.96 (t, 1.5H). MS (EI) for C₂₉H₂₉FN₄O₅S, found 565 (MH+).

6-(4-{[2-Ethyl-3-fluoro-4-(methylsulfonyl)phenyl]carbonyl}-2,3,4,5-tetrahydro-1,4-benzoxazepin-7-yl)-1H-benzimidazole-2-carboxylic acid. ¹H NMR (400 MHz, DMSO-d₆): δ 7.82-7.64 (m, 2H), 7.59-7.42 (m, 3H), 7.30 (d, 0.5H), 7.20-7.13 (m, 1H), 7.08 (s, 1H), 6.74 (d, 0.5H), 5.07-4.77 (m, 1H), 4.51-3.99 (m, 4H), 3.66-3.53 (m, 1H), 3.35 (s, 3H), 2.71-2.03 (m, 2H), 1.11 (t, 1.5H), 0.99 (t, 1.5H). MS (EI) for C₂₇H₂₄FN₃O₆S, found 538 (MH+).

6-(4-{[2-Ethyl-3-fluoro-4-(methylsulfonyl)phenyl]carbonyl}-2,3,4,5-tetrahydro-1,4-benzoxazepin-7-yl)-N-methyl-1H-benzimidazole-2-carboxamide. ¹H NMR (400 MHz, DMSO-d₆): δ 7.96-7.51 (m, 5H), 7.38 (dd, 0.5H), 7.25-7.19 (m, 1H), 7.15-7.10 (m, 1H), 6.68-6.62 (m, 0.5H), 5.10-4.84 (m, 1H), 4.53-4.03 (m, 4H), 3.75-3.62 (m, 1H), 3.27 (s, 3H), 3.00 (s, 3H), 2.83-2.73 (m, 0.5H), 2.58-2.38 (m, 1H), 2.16-2.05 (m, 0.5H), 1.18 (t, 1.5H), 1.06 (t, 1.5H). MS (EI) for C₂₈H₂₇FN₄O₅S, found 551 (MH+).

1-[6-(4-{[2-Ethyl-3-fluoro-4-(methylsulfonyl)-phenyl]carbonyl}-2,3,4,5-tetrahydro-1,4-benzoxazepin-7-yl)-1H-benzimidazol-2-yl]ethanone. ¹H NMR (400 MHz, DMSO-d₆): δ 13.40-13.26 (m, 1H), 7.90-7.84 (m, 0.5H), 7.81-7.68 (m, 2H), 7.63-7.49 (m, 2H), 7.45-7.25 (m, 1H), 7.19-7.05 (m, 2H), 6.80 (dd, 0.5H), 5.06-4.78 (m, 1H), 4.52-3.95 (m, 4H), 3.65-3.52 (m, 1H), 3.41-3.28 (m, 3H), 2.74-1.97 (m, 5H), 1.08 (t, 1.5), 0.96 (t, 1.5H). MS (EI) for C₂₈H₂₆FN₃O₅S, found 536 (MH+).

6-(4-{[2-Ethyl-3-fluoro-4-(methylsulfonyl)phenyl]carbonyl}-2,3,4,5-tetrahydro-1,4-benzoxazepin-7-yl)-1H-benzimidazole-2-carboxamide. ¹H NMR (400 MHz, DMSO-d₆): δ 7.78-7.66 (m, 1H), 7.56 (d, 1H), 7.45-7.34 (m, 2H), 7.27 (d, 1H), 7.21-7.01 (m, 4H), 6.66 (d, 1H), 5.02-4.75 (m, 1H), 4.46-4.00 (m, 4H), 3.63-3.50 (m, 1H), 3.34 (s, 3H), 2.67-2.02 (m, 1H), 1.08 (t, 1.5H), 0.97 (t, 1.5H). MS (EI) for C₂₇H₂₅FN₄O₅S, found 538 (MH+).

5-(4-{[3-Fluoro-2-methyl-4-(methylsulfonyl)phenyl]carbonyl}-2,3,4,5-tetrahydro-1,4-benzoxazepin-7-yl)pyrimidin-2-amine. ¹H NMR (400 MHz, DMSO-d₆): δ 8.52 (s, 1H), 8.43 (s, 1H), 7.82 (m, 1H), 7.75 (s, 1H), 7.50 (m, 1H), 7.30 (dd, 1H), 7.10 (t, 1H), 6.70 (s, 2H), 4.95 (m, 1H), 4.21 (m, 5H), 2.10 (s, 3H), 1.85 (s, 3H). MS (EI) for C₂₂H₂₁FN₄O₄S, found 457 (MH+).

4-{[2-Ethyl-3-fluoro-4-(methylsulfonyl)phenyl]carbonyl}-7-{2-[(2S)-pyrrolidin-2-ylmethyl]-1H-benzimidazol-6-yl}-2,3,4,5-tetrahydro-1,4-benzoxazepine. ¹H NMR (400 MHz, DMSO-d₆): δ 7.94 (s, 0.5H), 7.85-7.68 (m, 3H), 7.62-7.54 (d, 1H), 7.48-7.39 (m, 1H), 7.26 (d, 0.5H), 7.16-7.01 (m, 1.5H), 6.84 (s, 0.5H), 5.07-4.79 (m, 1H), 4.52-3.95 (m, 6H), 3.72-3.43 (m, 3H), 3.36-3.14 (m, 5H), 2.68-2.26 (m, 2H), 1.08 (t, 1.5H), 0.97 (t, 1.5H). MS (EI) for C₃₁H₃₃FN₄O₄S, found 577 (MH+).

N-Ethyl-6-(4-{[2-ethyl-3-fluoro-4-(methylsulfonyl)-phenyl]carbonyl}-2,3,4,5-tetrahydro-1,4-benzoxazepin-7-yl)-1H-benzimidazole-2-carboxamide. ¹H NMR (400 MHz, DMSO-d₆): δ 13.39-13.20 (m, 1H), 9.13-8.87 (m, 1H), 7.97-7.47 (m, 5H), 7.37-6.53 (m, 2H), 5.10-4.77 (m, 1H), 4.54-3.98 (m, 4H), 3.67-3.54 (m, 1H), 3.39-3.33 (m, 2H), 2.76-2.05 (m, 2H), 1.21-0.94 (m, 6H). MS (EI) for C₂₉H₂₉FN₄O₅S: 565 (MH+).

N-(1,1-Dimethylethyl)-6-(4-{[3-fluoro-2-methyl-4-(methylsulfonyl)phenyl]carbonyl}-2,3,4,5-tetrahydro-1,4-benzoxazepin-7-yl)-1H-benzimidazole-2-carboxamide. ¹H NMR (400 MHz, DMSO-d₆): δ 8.05-7.48 (m, 5H), 7.35-6.71 (m, 3H), 5.02-4.82 (m, 1H), 4.57-3.90 (m, 4H), 3.67-3.50 (m, 1H), 3.30 (m, 3H), 2.18-1.75 (m, 3H), 1.50-1.33 (m, 9H). MS (EI) for C₃₀H₃ᵢFN₄O₅S, found 579 (MH+).

6-(4-{[3-Fluoro-2-methyl-4-(methylsulfonyl)phenyl]carbonyl}-2,3,4,5-tetrahydro-1,4-benzoxazepin-7-yl)-N-phenyl-1H-benzimidazole-2-carboxamide. ¹H NMR (400 MHz, DMSO-d₆): δ 11.00-10.85 (m, 1H), 8.01-7.50 (m, 7H), 7.44-6.74 (m, 6H), 5.03-4.83 (m, 1H), 4.59-3.94 (m, 4H), 3.69-3.52 (m, 1H), 3.31 (s, 2H), 2.18-1.77 (m, 3H). MS (EI) for C₃₂H₂₇FN₄O₅S, found 599 (MH+).

6-(4-{[3-Fluoro-2-methyl-4-(methylsulfonyl)phenyl]carbonyl}-2,3,4,5-tetrahydro-1,4-benzoxazepin-7-yl)-N-propyl-1H-benzimidazole-2-carboxamide. ¹H NMR (400 MHz, DMSO-d₆): δ 9.07-8.95 (m, 1H), 7.94-7.48 (m, 5H), 7.33-6.73 (m, 2H), 5.02-4.83 (m, 1H), 4.55-3.93 (m, 4H), 3.66-3.50 (m, 1H), 3.34-3.26 (m, 3H), 2.16-1.76 (m, 3H), 1.66-1.51 (m, 2H), 0.94-0.84 (m, 3H). MS (EI) for C₂₉H₂₉FN₄O₅S, found 565 (MH+).

6-(4-{[3-Fluoro-2-methyl-4-(methylsulfonyl)phenyl]carbonyl}-2,3,4,5-tetrahydro-1,4-benzoxazepin-7-yl)-N,N-dimethyl-1H-benzimidazole-2-carboxamide. ¹H NMR (400 MHz, DMSO-d₆): δ 7.83-7.49 (m, 5H), 7.40-7.19 (m, 1.5H), 7.09 (d, 1H), 6.80 (br s, 0.5H), 5.00-4.82 (m, 1H), 4.56-3.93 (m, 4H), 3.69-3.54 (m, 4H), 3.34 (m, 3H), 2.13 (d, 1.5H), 1.79 (d, 1.5H). MS (EI) for C₂₈H₂₇FN₄O₅S, found 551 (MH+).

N-[6-(4-{[3-Fluoro-2-methyl-4-(methylsulfonyl)phenyl]-carbonyl}-2,3,4,5-tetrahydro-1,4-benzoxazepin-7-yl)-1H-benzimidazol-2-yl]acetamide. ¹H NMR (400 MHz, DMSO-d₆): δ 12.06-11.51 (m, 2H), 7.82-7.04 (m, 7.5H), 6.78-6.62 (m, 0.5H), 4.98-4.82 (m, 1H), 4.54-3.91 (m, 4H), 3.62-3.54 (m, 1H), 3.34 (s, 3H), 2.20-2.12 (m, 4.5H), 1.81-1.76 (m, 1.5H). MS (EI) for C₂₇H₂₅FN₄O₅S, found 537 (MH+).

N-Cyclopropyl-6-(4-{[3-fluoro-2-methyl-4-(methylsulfonyl)phenyl]carbonyl}-2,3,4,5-tetrahydro-1,4-benzoxazepin-7-yl)-1H-benzimidazole-2-carboxamide. ¹H NMR (400 MHz, DMSO-d₆): δ 9.13-8.95 (m, 1H), 7.94-7.48 (m, 5H), 7.33-6.72 (m, 2H), 5.01-4.83 (m, 1H), 4.55-3.93 (m, 4H), 3.66-3.50 (m, 1H), 3.33-3.24 (m, 3H), 2.94 (m, 1H), 2.15-1.77 (m, 3H), 1.73-1.62 (m, 2H). MS (EI) for C₂₉H₂₇FN₄O₅S, found 563 (MH+).

N-Cyclobutyl-6-(4-{[3-fluoro-2-methyl-4-(methylsulfonyl)phenyl]carbonyl}-2,3,4,5-tetrahydro-1,4-benzoxazepin-7-yl)-1H-benzimidazole-2-carboxamide. ¹H NMR (400 MHz, DMSO-d₆): δ 9.32-9.14 (m, 1H), 7.98-7.48 (m, 5H), 7.33-6.72 (m, 2H), 5.02-4.83 (m, 1H), 4.59-3.93 (m, 5H), 3.66-3.52 (m, 1H), 3.34-3.26 (m, 3H), 2.29-2.16 (m, 4H), 2.15-1.77 (m, 3H), 1.73-1.62 (m, 2H). MS (EI) for C₃₀H₂₉FN₄O₅S, found 577 (MH+).

N-[6-(4-{[3-Fluoro-2-methyl-4-(methylsulfonyl)-phenyl]carbonyl}-2,3,4,5-tetrahydro-1,4-benzoxazepin-7-yl)-1H-benzimidazol-2-yl]-N˜2˜,N˜2˜dimethylglycinamide. ¹H NMR (400 MHz, DMSO-d₆): δ 12.16-11.99 (m, 1H), 11.23 (br s, 1H), 7.83-7.38 (m, 5H), 7.32-7.21 (m, 1H), 7.15-7.04 (m, 1.5H), 6.78-6.62 (m, 0.5H), 4.99-4.82 (m, 1H), 4.54-3.91 (m, 4H), 3.62-3.55 (m, 1H), 3.39-3.28 (m, 3H), 3.25 (s, 2H), 2.33 (s, 6H), 2.13 (s, 1.5H), 1.82-1.76 (m, 1.5H). MS (EI) for C₂₉H₃₀FN₅O₅S, found 580 (MH+).

6-(4-{[3-Fluoro-2-methyl-4-(methylsulfonyl)phenyl]carbonyl}-2,3,4,5-tetrahydro-1,4-benzoxazepin-7-yl)-N-(1-methylethyl)-1H-benzimidazole-2-carboxamide. ¹H NMR (400 MHz, DMSO-d₆): δ 8.73-8.55 (m, 1H), 7.85-7.48 (m, 5H), 7.31-7.19 (m, 1.5H), 7.10 (dd, 1H), 6.75 (d, 0.5H), 5.01-4.83 (m, 1H), 4.54-3.92 (m, 5H), 3.64-3.53 (m, 1H), 3.34 (s, 1.5H), 3.30 (s, 1.5H), 2.13 (d, 1.5H), 1.80 (t, 1.5H), 1.25-1.20 (m, 6H). MS (EI) for C₂₉H₂₉FN₄O₅S, found 565 (MH+).

4-{[3-Fluoro-2-methyl-4-(methylsulfonyl)phenyl]carbonyl}-7-[2-(1H-imidazol-4-ylmethyl)-1H-benzimidazol-6-yl]-2,3,4,5-tetrahydro-1,4-benzoxazepine. ¹H NMR (400 MHz, DMSO-d₆): δ 7.80-7.64 (m, 3H), 7.56-7.40 (m, 3H), 7.31-7.20 (dd, 3.5H), 7.15-7.05 (m, 1.5H), 6.98 (s, 1H), 5.00-4.80 (m, 1H), 4.56-3.86 (m, 4H), 3.65-3.53 (m, 1H), 3.40 (m, 2H), 3.33 (d, 3H), 2.15-1.74 (d, 3H). MS (EI) for C₂₉H₂₆FN₅O₄S, found 560 (MH+).

N′-[6-(4-{[3-Fluoro-2-methyl-4-(methylsulfonyl)phenyl]-carbonyl}-2,3,4,5-tetrahydro-1,4-benzoxazepin-7-yl)-1H-benzimidazol-2-yl]-N,N-dimethylethane-1,2-diamine. ¹H NMR (400 MHz, Methanol-d₄): δ 7.91-7.79 (m, 1H), 7.62 (d, 0.5H), 7.51-7.44 (m, 1.5H), 7.32-7.18 (m, 3H), 7.10-7.02 (m, 1.5H), 6.50 (d, 0.5H), 4.52-4.33 (m, 3H), 4.16-3.93 (m, 2H), 3.70-3.63 (m, 1H), 3.59-3.54 (m, 2H), 3.26 (s, 1H), 3.21 (s, 2H), 2.81 (t, 2H), 2.48 (s, 6H), 2.20 (d, 1H), 1.80 (d, 2H). MS (EI) for C₂₉H₃₂FN₅O₄S, found 566 (MH+).

1,1-Dimethylethyl 7-(6-aminopyridin-3-yl)-2,3-dihydro-1,4-benzoxazepine-4(5H)-carboxylate. ¹H NMR (400 MHz, DMSO-d₆): δ 8.20 (m, 1H), 7.65 (m, 1H), 7.39 (m, 2H), 6.99 (m, 1H), 6.52 (dd, 1H), 6.04 (s, 2H), 4.47 (m, 2H), 4.04 (m, 2H), 3.71 (m, 2H), 1.32 (m, 9H). MS (EI) for C₁₉H₂₃N₃O₃, found 342 (MH+).

N-Cyclohexyl-6-(4-{[3-fluoro-2-methyl-4-(methylsulfonyl)phenyl]carbonyl}-2,3,4,5-tetrahydro-1,4-benzoxazepin-7-yl)-1H-benzimidazole-2-carboxamide. ¹H NMR (400 MHz, DMSO-d₆): δ 8.77-8.61 (m, 1H), 7.87-7.48 (m, 4H), 7.32-6.72 (m, 3H), 5.02-4.83 (m, 1H), 4.57-3.74 (m, 5H), 3.67-3.53 (m, 1H), 3.31 (s, 3H), 2.16-1.05 (m, 13H). MS (EI) for C₃₂H₃₃FN₄O₅S, found 605 (MH+).

4-{[2-Ethyl-3-fluoro-4-(methylsulfonyl)phenyl]carbonyl}-7-{2-[(2R)-pyrrolidin-2-ylmethyl]-1H-benzimidazol-6-yl}-2,3,4,5-tetrahydro-1,4-benzoxazepine. ¹H NMR (400 MHz, DMSO-d₆): δ 7.94 (s, 0.5H), 7.83-7.66 (m, 3.5H), 7.63-7.56 (m, 1H), 7.39 (d, 0.5H), 7.28 (d, 0.5H), 7.20 (d, 0.5H), 7.12 (d, 1H), 6.81 (s, 0.5H), 5.01-4.86 (m, 1H), 4.57-3.94 (m, 5H), 3.37-3.17 (m, 8H), 2.21-1.70 (m, 7H). MS (EI) for C₃₁H₃₃FN₄O₄S, found 563 (MH+).

Methyl 5-(4-{[3-fluoro-2-methyl-4-(methylsulfonyl)phenyl]carbonyl}-2,3,4,5-tetrahydro-1,4-benzoxazepin-7-yl)pyridine-2-carboxylate. ¹H NMR (400 MHz, DMSO-d₆): δ 9.04 (d, 0.5H), 8.84 (d, 0.5H), 8.28 (dd, 0.5H), 8.11 (d, 0.5H), 8.03 (d, 0.5H), 7.95 (dd, 0.5H), 7.87 (d, 0.5H), 7.75-7.67 (m, 2H), 7.27 (d, 0.5H), 7.16-7.10 (m, 1.5H), 6.96 (d, 0.5H), 4.99-4.89 (m, 1H), 4.54-3.95 (m, 4H), 3.90-3.86 (m, 3H), 3.59-3.54 (m, 1H), 2.10 (d, 1.5H), 1.76 (d, 1.5H). MS (EI) for C₂₅H₂₃FN₂O₆S, found 499 (MH+).

N-[2-(Dimethylamino)ethyl]-6-(4-{[3-fluoro-2-methyl-4-(methylsulfonyl)phenyl]carbonyl}-2,3,4,5-tetrahydro-1,4-benzoxazepin-7-yl)-1H-benzimidazole-2-carboxamide. ¹H NMR (400 MHz, Methanol-d₄): δ 7.95-7.79 (m, 2H), 7.77-7.51 (m, 3H), 7.43-7.10 (m, 2H), 6.81 (d, 1H), 5.08-4.86 (m, 1H), 4.55-3.95 (m, 4H), 3.76-3.62 (m, 3H), 3.26 (d, 3H), 2.87-2.81 (m, 2H), 2.51 (s, 6H), 2.23-1.80 (dd, 3H). MS (EI) for C₃₀H₃₂FN₅O₅S: 594 (MH+).

[5-(4-{[3-Fluoro-2-methyl-4-(methylsulfonyl)phenyl]-carbonyl}-2,3,4,5-tetrahydro-1,4-benzoxazepin-7-yl)pyridin-2-yl]methanol. ¹H NMR (400 MHz, DMSO-d₆): δ 8.80 (d, 0.5H), 8.59 (d, 0.5H), 8.09 (dd, 0.5H), 7.82-7.70 (m, 2H), 7.62-7.48 (m, 2H), 7.29 (d, 0.5H), 7.19 (d, 0.5H), 7.13-7.08 (m, 1H), 6.84 (d, 0.5H), 5.47 (q, 1H), 5.00-4.85 (m, 1H), 4.63-3.94 (m, 6H), 3.63-3.53 (m, 1H), 2.13 (s, 1.5H), 1.77 (s, 1.5H). MS (EI) for C₂₄H₂₃FN₂O₅S, found 471 (MH+).

5-(4-{[2-Ethyl-3-fluoro-4-(methylsulfonyl)phenyl]carbonyl}-9-fluoro-2,3,4,5-tetrahydro-1,4-benzoxazepin-7-yl)pyridin-2-amine. ¹H NMR (400 MHz, DMSO-d₆): δ 8.29 (d, 0.5H), 8.08 (d, 0.5H), 7.78-7.70 (m, 1.5H), 7.49-7.40 (m, 2H), 7.31 (d, 0.5H), 7.15 (d, 0.5H), 6.55-6.43 (m, 1.5H), 6.14 (s, 2H), 5.07-4.76 (m, 1H), 4.47-4.05 (m, 4H), 3.69-3.52 (m, 1H), 3.38 (s, 1.5H), 3.36 (s, 1.5H), 2.70-2.62 (m, 0.5H), 2.48-2.31 (m, 1H), 2.14-2.03 (m, 0.5H), 1.10 (t, 1.5H), 1.01 (t, 1.5H). MS (EI) for C₂₄H₂₃F₂N₃O₄S, found 488 (MH+).

5-(4-{[3-Fluoro-2-methyl-4-(methylsulfonyl)phenyl]carbonyl}-2,3,4,5-tetrahydro-1,4-benzoxazepin-7-yl)-N-methylpyridine-2-carboxamide. ¹H NMR (400 MHz, DMSO-d₆): δ 8.96-8.70 (m, 2H), 8.30-7.67 (m, 4H), 7.31-6.95 (m, 2H), 5.04-4.86 (m, 1H), 4.60-3.96 (m, 4H), 3.65-3.52 (m, 1H), 3.35 (d, 3H), 2.87-2.81 (t, 3H), 2.16-1.76 (dd, 3H). MS (EI) for C₂₅H₂₄FN₃O₅S, found 498 (MH+).

N-Ethyl-5-(4-{[3-fluoro-2-methyl-4-(methylsulfonyl)phenyl]-carbonyl}-2,3,4,5-tetrahydro-1,4-benzoxazepin-7-yl)pyridine-2-carboxamide. ¹H NMR (400 MHz, DMSO-d₆): δ 8.96-8.75 (m, 2H), 8.30-7.67 (m, 4H), 7.31-6.94 (m, 2H), 6.74 (m, 1H), 5.07-4.85 (m, 1H), 4.50-4.10 (m, 4H), 3.76-3.61 (m, 1H), 3.28 (d, 3H), 2.95 (d, 3H), 2.80-2.08 (m, 2H), 1.20-1.03 (m, 3H). MS (EI) for C₂₆H₂₆FN₃O₅S, found 512 (MH+).

4-{[2-Ethyl-3-fluoro-4-(methylsulfonyl)phenyl]carbonyl}-7-[3-(methyloxy)phenyl]-2,3,4,5-tetrahydro-1,4-benzoxazepine. ¹H NMR (400 MHz, DMSO-d₆): δ 7.78-7.69 (m, 1.5H), 7.57-7.50 (m, 1H), 7.40-6.82 (, m, 6.5H), 5.05-4.79 (m, 1H), 4.50-4.06 (m, 4H), 3.83 (s, 1.5H), 3.79 (s, 1.5H), 3.62-3.56 (m, 1H), 3.35 (s, 3H), 2.70-2.60 (m, 0.5H), 2.48-2.32 (m, 1H), 2.17-2.06 (m, 0.5H), 1.09 (t, 1.5H), 1.00 (t, 1.5H). MS (EI) for C₂₆H₂₆FNO₅S, found 484 (MH+).

N′-[4-(4-{[2-Ethyl-3-fluoro-4-(methylsulfonyl)phenyl]-carbonyl}-2,3,4,5-tetrahydro-1,4-benzoxazepin-7-yl)-2-(methyloxy)phenyl]-N,N-dimethylethane-1,2-diamine. ¹H NMR (400 MHz, DMSO-d₆): δ 7.80-7.71 (m, 1H), 7.61 (d, 0.5H), 7.48-7.42 (m, 1H), 7.27 (d, 0.5H), 7.16-7.08 (m, 1.5H), 7.04-6.99 (m, 1H), 6.92-6.87 (m, 1H), 6.74 (d, 0.5H), 6.61 (d, 0.5H), 6.54 (d, 0.5H), 5.03-4.74 (m, 2H), 4.42-3.99 (m, 4H), 3.89 (s, 1.5H), 3.83 (s, 1.5H), 3.61-3.53 (m, 2H), 3.16-3.08 (m, 2H), 2.20-2.15 (m, 6H), 1.10 (t, 1.5H), 1.02 (t, 1.5H). MS (EI) for C₃₀H₃₆FN₃O₅S, found 570 (MH+).

2-{[2-(Dimethylamino)ethyl]oxy}-4-(4-{[2-ethyl-3-fluoro-4-(methylsulfonyl)phenyl]carbonyl}-2,3,4,5-tetrahydro-1,4-benzoxazepin-7-yl)aniline. ¹H NMR (400 MHz, DMSO-d₆): δ 7.79-7.71 (m, 1H), 7.58 (d, 0.5H), 7.46-7.40 (m, 1H), 7.27 (d, 0.5H), 7.16-7.10 (m, 1H), 7.04-6.98 (m, 1.5H), 6.93 (d, 0.5H), 6.78-6.63 (m, 2H), 5.01-4.75 (m, 3H), 4.39 (s, 1H), 4.26-4.00 (m, 5H), 3.62-3.52 (m, 1H), 2.70-2.61 (m, 2.5H), 2.48-2.35 (m, 1H), 2.27-2.20 (m, 6H), 2.18-2.05 (m, 0.5H), 1.10 (t, 1.5H), 1.01 (t, 1.5H). MS (EI) for C₂₉H₃₄FN₃O₅S, found 556 (MH+).

4-(4-{[2-Ethyl-3-fluoro-4-(methylsulfonyl)phenyl]carbonyl}-2,3,4,5-tetrahydro-1,4-benzoxazepin-7-yl)-N-2-dimethylbenzamide. ¹H NMR (400 MHz, DMSO-d₆): δ 8.22-8.15 (m, 1H), 7.77-7.69 (m, 1.5H), 7.57-7.48 (m, 2H), 7.38 (d, 0.5H), 7.33-7.24 (m, 2H), 7.12-7.04 (m, 1.5H), 6.82 (d, 0.5H), 5.03-4.79 (m, 1H), 4.42 (s, 1H), 4.31-4.04 (m, 3H), 3.60-3.53 (m, 1H), 3.32 (s, 3H), 2.76-2.71 (m, 3H), 2.69-2.58 (m, 0.5H), 2.45-2.30 (m, 4H), 2.15-2.05 (m, 0.5H), 1.08 (t, 1.5H), 0.98 (t, 1.5H). MS (EI) for C₂₈H₂₉FN₂O₅S, found 525 (MH+).

1-[4-(4-{[2-Ethyl-3-fluoro-4-(methylsulfonyl)phenyl]-carbonyl}-2,3,4,5-tetrahydro-1,4-benzoxazepin-7-yl)phenyl]-3-methylurea. ¹H NMR (400 MHz, DMSO-d₆): δ 8.61 (d, 1H), 7.81-7.70 (m, 1H), 7.62 (d, 0.5H), 7.56-7.39 (m, 4H), 7.32-7.26 (m, 1.5H), 7.15 (d, 0.5H), 7.04 (dd, 1H), 6.66 (d, 0.5H), 6.06-5.96 (m, 1H), 5.03-4.75 (m, 1H), 4.46-3.98 (m, 4H), 3.65-3.52 (m, 1H), 3.37 (s, 1.5H), 3.35 (s, 1.5H), 2.69-2.62 (m, 3.5H), 2.48-2.28 (m, 1H), 2.09-1.99 (m, 0.5H), 1.09 (t, 1.5H), 0.98 (t, 1.5H). MS (EI) for C₂₇H₂₈FN₃O₅S, found 526 (MH+).

N-[2-(Dimethylamino)ethyl]-4-(4-{[2-ethyl-3-fluoro-4-(methylsulfonyl)phenyl]carbonyl}-2,3,4,5-tetrahydro-1,4-benzoxazepin-7-yl)benzamide. ¹H NMR (400 MHz, CDCl₃): δ 8.50 (s, 0.5H), 8.00-7.70 (m, 4H), 7.61-7.51 (m, 2H), 7.24-7.10 (m, 2H), 7.14-7.09 (d, 2H), 6.69 (s, 0.5H), 5.08-4.84 (dd, 1H), 4.47 (s, 1H), 4.38-4.08 (m, 3H), 3.77-3.66 (m, 3H), 3.30-3.25 (d, 3H), 3.24-3.18 (m, 2H), 2.84 (s, 5H), 2.83-2.09 (m, 2H), 1.21-1.03 (dt, 3H). MS (EI) for C₃₀H₃₄FN₃O₅S found 568 (MH+).

4-(4-{[2-Ethyl-3-fluoro-4-(methylsulfonyl)phenyl]carbonyl}-2,3,4,5-tetrahydro-1,4-benzoxazepin-7-yl)-N-(2-methoxyethyl)benzamide. ¹H NMR (400 MHz, CDCl₃): δ 7.90-7.80 (m, 3H), 7.70-7.64 (m, 1H), 7.54-7.42 (m, 2H), 7.17-7.06 (m, 2H), 6.69 (s, 1H), 5.07-4.84 (dd, 1H), 4.46-4.20 (m, 3H), 3.72-3.56 (m, 6H), 3.42 (s, 3H), 3.25 (d, 3H), 2.82-2.22 (m, 2H), 1.24-1.11 (dt, 3H). MS (EI) for C₂₉H₃₁FN₂O₆S, found 555 (MH+).

5-{4-[(2-Methylphenyl)carbonyl]-2,3,4,5-tetrahydro-1,4-benzoxazepin-7-yl}pyridin-2-amine. ¹H NMR (400 MHz, DMSO-d₆): δ 8.72 (m, 2H), 8.51 (m, 2H), 7.70 (br s, 2H), 7.48 (m, 2H), 7.38-7.28 (m, 2H), 7.25-7.15 (m, 2H), 4.65 (s, 2H), 4.17 (s, 2H), 3.86 (m, 2H), 3.26 (s, 3H). MS (EI) for C₂₂H₂ᵢN₃O₂ found 360.4 (MH+).

4-{[2-Ethyl-4-(ethylsulfonyl)-3-fluorophenyl]carbonyl}-7-(1H-pyrazol-5-yl)-2,3,4,5-tetrahydro-1,4-benzoxazepine. ¹H NMR (400 MHz, DMSO-d6): δ 7.85-7.25 (m, 4H), 7.17-6.39 (m, 3H), 5.04-4.75 (m, 1H), 4.48-3.98 (m, 4H), 3.65-3.52 (m, 1H), 3.49-3.33 (m, 2H), 2.70-2.01 (m, 2H), 1.25-0.92 (m, 6H). MS (EI) for C₂₃H₂₄FN₃O₄S, found 458 (MH+).

4-{[2-Ethyl-3-fluoro-4-(methylsulfonyl)phenyl]carbonyl}-7-[4-(1H-pyrazol-3-yl)phenyl]-2,3,4,5-tetrahydro-1,4-benzoxazepine. ¹H NMR (400 MHz, DMSO-d₆): δ 13.39-12.87 (m, 1H), 7.96-7.69 (m, 5H), 7.63-7.43 (m, 2.5H), 7.29 (d, 0.5H), 7.16 (d, 0.5H), 7.12-7.06 (m, 1H), 6.88-6.70 (m, 1.5H), 5.07-4.80 (m, 1H), 4.52-3.98 (m, 4H), 3.66-3.53 (m, 1H), 3.40-3.35 (m, 3H), 2.72-2.29 (m, 1.5H), 2.14-2.01 (m, 0.5H), 1.10 (t, 1.5H), 0.99 (t, 1.5H). MS (EI) for C₂₈H₂₆FN₃O₄S, found 520 (MH+).

4-{[2-Ethyl-4-(ethylsulfonyl)-3-fluorophenyl]carbonyl}-7-[4-(1H-pyrazol-3-yl)phenyl]-2,3,4,5-tetrahydro-1,4-benzoxazepine. ¹H NMR (400 MHz, DMSO-d₆): δ 12.92 (br s, 1H), 7.95-7.68 (m, 5H), 7.62-7.43 (m, 2.5H), 7.30 (d, 0.5H), 7.16-7.06 (m, 1.5H), 6.80-6.71 (m, 1.5H), 5.06-4.81 (m, 1H), 4.49-4.41 (m, 4H), 3.65-3.51 (m, 1H), 3.45-3.38 (m, 2H), 2.67-2.05 (m, 2H), 1.20-1.13 (m, 3H), 1.09 (t, 1.5H), 0.99 (t, 1.5H). MS (EI) for C₂₉H₂₈FN₃O₄S, found 534 (MH+).

4-{[2-Ethyl-4-(ethylsulfonyl)-3-fluorophenyl]carbonyl}-7-[4-(1H-imidazol-2-yl)phenyl]-2,3,4,5-tetrahydro-1,4-benzoxazepine. ¹H NMR (400 MHz, DMSO-d₆): δ 12.54 (s, 1H), 8.07-7.91 (m, 2H), 7.80-7.24 (m, 5H), 7.22-6.73 (m, 3H), 5.06-4.77 (m, 1H), 4.43 (s, 1H), 4.33-4.04 (m, 3H), 3.64-3.53 (m, 1H), 3.47-3.34 (m, 3H), 2.71-2.02 (m, 2H), 1.19-0.93 (m, 6H). MS (EI) for C₂₉H₂₈FN₃O₄S, found 534 (MH+).

4-{[2-Ethyl-4-(ethylsulfonyl)-3-fluorophenyl]carbonyl}-7-[4-(1H-imidazol-4-yl)phenyl]-2,3,4,5-tetrahydro-1,4-benzoxazepine. ¹H NMR (400 MHz, DMSO-d₆): δ 12.18 (s, 1H), 7.91-7.29 (m, 8H), 7.18-6.70 (m, 2H), 5.06-4.80 (m, 1H), 4.44 (s, 1H), 4.34-4.04 (m, 3H), 3.66-3.54 (m, 1H), 3.42 (m, 3H), 2.71-2.05 (m, 2H), 1.21-0.95 (m, 6H). MS (EI) for C₂₉H₂₈FN₃O₄S, found 534 (MH+).

4-{[2-Ethyl-3-fluoro-4-(methylsulfonyl)phenyl]carbonyl}-7-{4-[4-(trifluoromethyl)-1H-imidazol-2-yl]phenyl}-2,3,4,5-tetrahydro-1,4-benzoxazepine. ¹H NMR (400 MHz, DMSO-d₆): δ 13.26 (br s, 1H), 8.07 (d, 1H), 7.99 (d, 1H), 7.94 (d, 1H), 7.85-7.71 (m, 2.5H), 7.66-7.54 (m, 2H), 7.29 (d, 0.5H), 7.17-7.07 (m, 1.5H), 6.86 (d, 0.5H), 5.07-4.82 (m, 1H), 4.52-4.00 (m, 4H), 3.66-3.54 (m, 1H), 3.39 (s, 1.5H), 3.36 (s, 1.5H), 2.71-2.29 (m, 1.5H), 2.14-2.02 (m, 0.5H), 1.10 (t, 1.5H), 1.00 (t, 1.5H). MS (EI) for C₂₉H₂₅F₃N₃O₄S, found 588 (MH+).

4-{[3-Fluoro-2-methyl-4-(methylsulfonyl)phenyl]carbonyl}-7-[4-(1H-imidazol-4-yl)phenyl]-2,3,4,5-tetrahydro-1,4-benzoxazepine. ¹H NMR (400 MHz, DMSO-d₆): δ 12.21 (s, 1H), 7.92-7.27 (m, 9H), 7.23-6.73 (m, 2H), 5.01-4.83 (m, 1H), 4.55-3.91 (m, 4H), 3.64-3.51 (m, 1H), 3.37 (s, 3H), 2.16-1.75 (m, 3H). MS (EI) for C₂₇H₂₄FN₃O₄S, found 506 (MH+).

4-{[2-Ethyl-3-fluoro-4-(methylsulfonyl)phenyl]carbonyl}-7-[4-(3-methyl-1H-pyrazol-5-yl)phenyl]-2,3,4,5-tetrahydro-1,4-benzoxazepine. ¹H NMR (400 MHz, DMSO-d₆): δ 7.90-7.26 (m, 7H), 7.19-6.44 (m, 3H), 5.10-4.78 (m, 1H), 4.52-3.96 (m, 4H), 3.66-3.52 (m, 1H), 2.73-2.00 (m, 5H), 1.15-0.93 (m, 3H). MS (EI) for C₂₉H₂₈FN₃O₄S, found 534 (MH+).

4-{[2-Ethyl-4-(ethylsulfonyl)-3-fluorophenyl]carbonyl}-7-[4-(3-methyl-1H-pyrazol-5-yl)phenyl]-2,3,4,5-tetrahydro-1,4-benzoxazepine. ¹H NMR (400 MHz, DMSO-d₆): δ 12.52 (s, 1H), 7.84-7.67 (m, 1H), 7.60-7.51 (m, 1H), 7.44 (m, 1H), 7.33-7.11 (m, 1H), 7.10-6.72 (m, 2H), 6.51-6.45 (m, 1H), 5.07-4.81 (m, 1H), 4.44 (m, 1H), 4.34-4.06 (m, 3H), 3.66-3.54 (m, 1H), 3.47-3.33 (m, 2H), 2.69-2.04 (m, 5H), 1.20-0.95 (m, 6H). MS (EI) for C₃₀H₃₀FN₃O₄S, found 548 (MH+).

4-{[3-Fluoro-2-methyl-4-(methylsulfonyl)phenyl]carbonyl}-7-[4-(1H-imidazol-2-yl)phenyl]-2,3,4,5-tetrahydro-1,4-benzoxazepine. ¹H NMR (400 MHz, DMSO-d₆): δ 8.07-7.92 (m, 2H), 7.82-7.40 (m, 2H), 7.65-7.56 (m, 1H), 7.54-7.48 (m, 1H), 7.51-7.44 (m, 1H), 7.33-7.15 (m, 3H), 7.13-6.79 (m, 2H), 5.01-4.84 (m, 1H), 4.57-3.90 (m, 4H), 3.64-3.53 (m, 1H), 3.37 (s, 3H), 2.16-1.76 (m, 3H). MS (EI) for C₂₇H₂₄FN₃O₄S, found 506 (MH+).

4-{[2-Ethyl-3-fluoro-4-(methylsulfonyl)phenyl]carbonyl}-7-[4-(5-methyl-4H-1,2,4-triazol-3-yl)phenyl]-2,3,4,5-tetrahydro-1,4-benzoxazepine. ¹H NMR (400 MHz, DMSO-d₆): δ 13.77 (br s, 1H), 8.06 (d, 1H), 8.00 (d, 1H), 7.82-7.71 (m, 2.5H), 7.64-7.49 (m, 2H), 7.29 (d, 0.5H), 7.17-7.07 (m, 1.5H), 6.80 (d, 0.5H), 5.08-4.80 (m, 1H), 4.51-4.00 (m, 4H), 3.66-3.52 (m, 1H), 3.40-3.36 (m, 3H), 2.72-2.00 (m, 5H), 1.10 (t, 1.5H), 0.99 (t, 1.5H). MS (EI) for C₂₈H₂₇FN₄O₄S, found 535 (MH+).

4-{[2-Ethyl-3-fluoro-4-(methylsulfonyl)phenyl]carbonyl}-7-(4-imidazo[2,1-b][1,3]thiazol-6-ylphenyl)-2,3,4,5-tetrahydro-1,4-benzoxazepine. ¹H NMR (400 MHz, DMSO-d₆): δ 8.33-8.24 (m, 1H), 7.99-7.89 (m, 2H), 7.88-7.69 (m, 4H), 7.62-7.53 (m, 1H), 7.51-7.44 (m, 1H), 7.32-7.25 (m, 1H), 7.19-6.77 (m, 2H), 5.07-4.79 (m, 1H), 4.53-3.99 (m, 4H), 3.66-3.52 (m, 1H), 2.73-2.00 (m, 2H), 1.14-0.94 (m, 3H). MS (EI) for C₂₇H₂₄FN₃O₄S, found 576 (MH+).

4-{[2-Ethyl-4-(ethylsulfonyl)-3-fluorophenyl]carbonyl}-7-(4-imidazo[2,1-b][1,3]thiazol-6-ylphenyl)-2,3,4,5-tetrahydro-1,4-benzoxazepine. ¹H NMR (400 MHz, DMSO-d₆): δ 8.32-8.25 (m, 1H), 7.99-7.82 (m, 3H), 7.77-7.68 (m, 2H), 7.63-7.51 (m, 1H), 7.48-7.42 (m, 1H), 7.34-7.26 (m, 2H), 7.17-6.71 (m, 2H), 5.08-4.80 (m, 1H), 4.44 (s, 1H), 4.34-4.06 (m, 3H), 3.67-3.54 (m, 1H), 3.47-3.34 (m, 2H), 2.70-2.05 (m, 2H), 1.21-0.95 (m, 6H). MS (EI) for C₃₁H₂₈FN₃O₄S₂, found 590 (MH+).

4-{[2-Ethyl-3-fluoro-4-(methylsulfonyl)phenyl]carbonyl}-7-[4-(4-methyl-1H-imidazol-2-yl)phenyl]-2,3,4,5-tetrahydro-1,4-benzoxazepine. ¹H NMR (400 MHz, DMSO-d₆): δ 8.12 (d, 1H), 8.07-7.99 (m, 2H), 7.86 (d, 0.5H), 7.78-7.65 (m, 3H), 7.53 (d, 1H), 7.29 (d, 0.5H), 7.17-7.07 (m, 1.5H), 6.96 (d, 0.5H), 5.08-4.85 (m, 1H), 4.54-4.02 (m, 4H), 3.65-3.56 (m, 1H), 3.38-3.35 (m, 3H), 2.71-2.04 (m, 5H), 1.10 (t, 1.5H), 0.99 (t, 1.5H). MS (EI) for C₂₉H₂₈FN₃O₄S, found 534 (MH+).

5-(4-{[2-Ethyl-3-fluoro-4-(methylsulfonyl)phenyl]carbonyl}-2,3,4,5-tetrahydro-1,4-benzoxazepin-7-yl)-2,3-dihydro-1H-isoindol-1-one. ¹H NMR (400 MHz, DMSO-d₆): δ 8.58 (s, 1H), 7.87 (s, 0.5H), 7.79-7.54 (m, 4.5H), 7.29 (d, 0.5H), 7.13-7.09 (m, 2H), 6.82 (d, 0.5H), 5.03 (d, 0.5H), 4.86 (d, 0.5H), 4.45 (s, 2H), 4.39 (s, 1H), 4.31-4.27 (m, 1H), 4.20-4.14 (m, 1H), 4.13-4.08 (m, 1H), 3.63-3.57 (m, 1H), 3.39 (s, 3H), 2.48-2.32 (m, 1H), 2.17-2.07 (m, 1H), 1.11-0.99 (m, 3H). MS (EI) for C₂₇H₂₅FN₂O₅S, found 509 (MH+).

7-[4-(4,5-Dimethyl-1H-imidazol-2-yl)phenyl]-4-{[2-ethyl-4-(ethylsulfonyl)-3-fluorophenyl]carbonyl}-2,3,4,5-tetrahydro-1,4-benzoxazepine. ¹H NMR (400 MHz, DMSO-d₆): δ 11.96 (s, 1H), 8.02-7.92 (m, 1H), 7.91-7.85 (m, 1H), 7.84-7.75 (m, 1H), 7.73-7.67 (m, 1H), 7.65-7.49 (m, 2H), 7.30-6.77 (m, 3H), 5.05-4.81 (m, 1H), 4.44 (s, 1H), 4.35-4.02 (m, 3H), 3.61-3.50 (m, 1H), 3.45-3.35 (m, 2H), 2.19-2.13 (m, 6H), 1.19-0.88 (m, 6H). MS (EI) for C₃₁H₂₈FN₃O₄S₂, found 562.2 (MH+).

4-{[2-Ethyl-3-fluoro-4-(methylsulfonyl)phenyl]carbonyl}-7-[4-(1H-1,2,3-triazol-5-yl)phenyl]-2,3,4,5-tetrahydro-1,4-benzoxazepine. ¹H NMR (400 MHz, DMSO-d6): δ 8.45-8.33 (m, 1H), 7.96 (d, 1H), 7.88 (d, 1H), 7.81-7.72 (m, 2.5H), 7.63-7.52 (m, 2H), 7.29 (d, 0.5H), 7.17 (d, 0.5H), 7.10 (dd, 1H), 6.83 (d, 0.5H), 5.07-4.81 (m, 1H), 4.53-3.97 (m, 3H), 3.66-3.53 (m, 1H), 3.38-3.35 (m, 3H), 2.71-2.02 (m, 4H), 1.10 (t, 1.5H), 0.99 (t, 1.5H). MS (EI) for C₂₇H₂₅FN₄O₄S, found 521 (MH+).

5-[4-(4-{[2-Ethyl-3-fluoro-4-(methylsulfonyl)phenyl]-carbonyl}-2,3,4,5-tetrahydro-1,4-benzoxazepin-7-yl)phenyl]pyridin-2-amine. ¹H NMR (400 MHz, DMSO-d₆): δ 8.31-8.22 (m, 1H), 7.82-7.24 (m, 8H), 7.17-6.47 (m, 3H), 5.06-4.77 (m, 1H), 4.52-3.93 (m, 4H), 3.66-3.51 (m, 1H), 3.36 (s, 3H), 2.72-1.97 (m, 2H), 1.12-0.91 (m, 3H). MS (EI) for C₃₀H₂₈FN₃O₄S, found 546 (MH+).

5-[4-(4-{[2-Ethyl-4-(ethylsulfonyl)-3-fluorophenyl]carbonyl}-2,3,4,5-tetrahydro-1,4-benzoxazepin-7-yl)phenyl]pyridin-2-amine. ¹H NMR (400 MHz, DMSO-d₆): δ 8.34-8.24 (m, 1H), 7.82-7.27 (m, 8H), 7.17-6.72 (m, 2H), 6.58-6.53 (m, 1H), 6.11 (s, 2H), 5.07-4.78 (m, 1H), 4.45 (s, 1H), 4.36-4.02 (m, 3H), 3.66-3.52 (m, 1H), 3.47-3.33 (m, 2H), 2.73-2.02 (m, 2H), 1.20-0.94 (m, 6H). MS (EI) for C₃₁H₃₀FN₃O₄S, found 560 (MH+).

4-{[2-Ethyl-4-(ethylsulfonyl)-3-fluorophenyl]carbonyl}-7-[4-(1-methyl-1H-imidazol-5-yl)phenyl]-2,3,4,5-tetrahydro-1,4-benzoxazepine. ¹H NMR (400 MHz, DMSO-d₆): δ 7.81-7.68 (m, 3H), 7.64-7.28 (M, 5H), 7.17-6.75 (m, 3H), 5.07-4.81 (m, 1H), 4.54-3.96 (m, 4H), 3.77-3.66 (m, 3H), 3.63-3.55 (m, 1H), 3.47-3.36 (m, 2H), 2.70-2.03 (m, 2H), 1.20-0.94 (m, 6H). MS (EI) for C₃₀H₃₀FN₃O₄S, found 548 (MH+).

4-{[2-Ethyl-3-fluoro-4-(methylsulfonyl)phenyl]carbonyl}-7-[4-(4H-1,2,4-triazol-4-yl)phenyl]-2,3,4,5-tetrahydro-1,4-benzoxazepine. ¹H NMR (400 MHz, DMSO-d₆): δ 9.21 (s, 1H), 9.18 (s, 1H), 7.78 (m, 7H), 7.20 (m, 2H), 5.00 (dd, 1H), 4.30 (m, 5H), 3.23 (s, 3H), 2.72 (q, 2H), 1.20 (t, 3H). MS (EI) for C₂₇H₂₅FN₄O₄S, found 521 (MH+).

4-{[2-Ethyl-4-(ethylsulfonyl)-3-fluorophenyl]carbonyl}-7-[4-(4H-1,2,4-triazol-4-yl)phenyl]-2,3,4,5-tetrahydro-1,4-benzoxazepine. ¹H NMR (400 MHz, DMSO-d₆): δ 9.21 (s, 1H), 9.18 (s, 1H), 7.78 (m, 7H), 7.20 (m, 2H), 5.00 (dd, 1H), 4.30 (m, 5H), 3.27 (m, 4H), 1.25 (m, 6H). MS (EI) for C₂₇H₂₇FN₄O₄S, found 535 (MH+).

4-{[2-Ethyl-3-fluoro-4-(methylsulfonyl)phenyl]carbonyl}-7-[4-(1-methyl-1H-imidazol-5-yl)phenyl]-2,3,4,5-tetrahydro-1,4-benzoxazepine. ¹H NMR (400 MHz, DMSO-d₆): δ 7.82-7.69 (m, 3H), 7.64-7.26 (m, 5H), 7.19-6.78 (m, 3H), 5.06-4.79 (m, 1H), 4.54-3.96 (m, 4H), 3.77-3.66 (m, 3H), 3.63-3.54 (m, 1H), 3.36 (s, 3H), 2.71-1.97 (m, 2H), 1.14-0.93 (m, 3H). MS (EI) for C₂₉H₂₈FN₃O₄S, found 534 (MH+).

4-{[2-Ethyl-3-fluoro-4-(methylsulfonyl)phenyl]carbonyl}-7-[4-(1-methyl-1H-imidazol-4-yl)phenyl]-2,3,4,5-tetrahydro-1,4-benzoxazepine. ¹H NMR (400 MHz, DMSO-d₆): δ 7.85-7.62 (m, 7H), 7.59-7.50 (m, 1H), 7.45-7.39 (m, 1H), 7.31-7.13 (m, 1H), 7.10-6.75 (m, 1H), 5.07-4.78 (m, 1H), 4.51-3.88 (m, 4H), 3.70 (m, 3H), 3.39 (s, 3H), 2.96-2.01 (m, 2H), 1.21-0.95 (m, 3H). MS (EI) for C₂₉H₂₈FN₃O₄S, found 534 (MH+).

4-{[2-Ethyl-3-fluoro-4-(methylsulfonyl)phenyl]carbonyl}-7-[6-(1H-imidazol-2-yl)pyridin-3-yl]-2,3,4,5-tetrahydro-1,4-benzoxazepine. ¹H NMR (400 MHz, DMSO-d₆): δ 12.87 (br s, 1H), 8.92 (d, 0.5H), 8.74 (d, 0.5H), 8.19 (dd, 0.5H), 8.11 (d, 0.5H), 8.04 (d, 0.5H), 7.92-7.82 (m, 1H), 7.80-7.65 (m, 2H), 7.33-7.10 (m, 4H), 6.96 (d, 0.5H), 5.07-4.83 (m, 1H), 4.54-4.01 (m, 4H), 3.66-3.53 (m, 1H), 3.40-3.35 (m, 3H), 2.71-2.06 (m, 2H), 1.10 (t, 1.5H), 1.00 (t, 1.5H). MS (EI) for C₂₇H₂₅FN₄O₄S, found 521 (MH+).

4-{[2-Ethyl-3-fluoro-4-(methylsulfonyl)phenyl]carbonyl}-7-[5-(1H-imidazol-2-yl)pyridin-2-yl]-2,3,4,5-tetrahydro-1,4-benzoxazepine. ¹H NMR (400 MHz, DMSO-d₆): δ 12.77 (br s, 1H), 9.23-9.11 (m, 1H), 8.36-7.94 (m, 3H), 7.81-7.70 (m, 2H), 7.39-7.05 (m, 4H), 5.08-4.84 (m, 1H), 4.52-4.01 (m, 4H), 3.67-3.55 (m, 1H), 3.38 (m, 3H), 2.71-2.04 (m, 2H), 1.13-0.96 (m, 3H). MS (EI) for C₂₇H₂₅FN₄O₄S, found 521 (MH+).

4-{[2-Ethyl-3-fluoro-4-(methylsulfonyl)phenyl]carbonyl}-7-[4-(1-methyl-1H-imidazol-2-yl)phenyl]-2,3,4,5-tetrahydro-1,4-benzoxazepine. ¹H NMR (400 MHz, DMSO-d₆): δ 11.98 (s, 1H), 7.83-7.70 (m, 4H), 7.65-7.53 (m, 2H), 7.35-7.27 (m, 2H), 7.18-6.81 (m, 3H), 5.08-4.81 (m, 1H), 4.53-3.98 (m, 4H), 3.84-3.76 (m, 3H), 3.65-3.55 (m, 1H), 3.36 (s, 3H), 2.69-2.03 (m, 2H). MS (EI) for C₂₉H₂₈FN₃O₄S, found 534 (MH+).

4-{[2-Ethyl-4-(ethylsulfonyl)-3-fluorophenyl]carbonyl}-7-[4-(1-methyl-1H-imidazol-2-yl)phenyl]-2,3,4,5-tetrahydro-1,4-benzoxazepine. ¹H NMR (400 MHz, DMSO-d₆): δ 7.82-7.69 (m, 4H), 7.65-7.55 (m, 1H), 7.33-0.726 (m, 2H), 7.16 (m, 3H), 5.07-4.82 (m, 1H), 4.46 (s, 1H), 4.36-4.05 (m, 3H), 3.82-3.77 (m, 3H), 3.65-3.56 (m, 1H), 3.47-3.36 (m, 3H), 2.70-2.05 (m, 2H), 1.20-0.95 (m, 6H). MS (EI) for C₃₀H₃₀FN₃O₄S, found 548 (MH+).

1-[4-(4-{[2-Ethyl-3-fluoro-4-(methylsulfonyl)phenyl]-carbonyl}-2,3,4,5-tetrahydro-1,4-benzoxazepin-7-yl)phenyl]-1,2-dihydro-3H-1,2,4-triazol-3-one. ¹H NMR (400 MHz, DMSO-d₆): δ 11.49 (s, 1H), 8.96 (d, 1H), 7.87-7.70 (m, 4.5H), 7.63-7.54 (m, 2H), 7.29 (d, 0.5H), 7.15 (d, 0.5H), 7.09 (dd, 1H), 6.81 (d, 0.5H), 5.07-4.81 (m, 1H), 4.51-4.00 (m, 4H), 3.66-3.53 (m, 1H), 3.40-3.35 (m, 3H), 2.71-2.02 (m, 2H), 1.10 (t, 1.5H), 0.99 (t, 1.5H). MS (EI) for C₂₇H₂₅FN₄O₅S, found 537 (MH+).

4-[4-(4-{[2-Ethyl-3-fluoro-4-(methylsulfonyl)phenyl]-carbonyl}-2,3,4,5-tetrahydro-1,4-benzoxazepin-7-yl)phenyl]-2,4-dihydro-3H-1,2,4-triazol-3-one. ¹H NMR (400 MHz, DMSO-d₆): δ 12.03 (d, 1H), 8.48-8.41 (m, 1H), 7.83-7.68 (m, 4.5H), 7.62-7.55 (m, 2H), 7.29 (d, 0.5H), 7.16 (d, 0.5H), 7.10 (dd, 1H), 6.84 (d, 0.5H), 5.07-4.81 (m, 1H), 4.53-3.99 (m, 4H), 3.65-3.52 (m, 1H), 3.37-3.34 (m, 3H), 2.75-2.01 (m, 2H), 1.10 (t, 1.5H), 0.99 (t, 1.5H). MS (EI) for C₂₇H₂₅FN₄O₅S, found 537 (MH+).

4-{[3-Fluoro-2-methyl-4-(methylsulfonyl)phenyl]carbonyl}-7-[4-(1-methyl-1H-imidazol-4-yl)phenyl]-2,3,4,5-tetrahydro-1,4-benzoxazepine. ¹H NMR (400 MHz, DMSO-d₆): δ 7.87-7.26 (m, 9H), 7.24-6.71 (m, 2H), 5.00-4.83 (m, 1H), 4.55-3.90 (m, 4H), 3.70 (m, 3H), 3.64-3.53 (m, 1H), 3.37 (s, 3H), 2.16-1.74 (m, 3H). MS (EI) for C₂₈H₂₆FN₃O₄S, found 520 (MH+).

{5-[4-(4-{[2-Ethyl-3-fluoro-4-(methylsulfonyl)phenyl]-carbonyl}-2,3,4,5-tetrahydro-1,4-benzoxazepin-7-yl)phenyl]-1H-imidazol-2yl}methanol. ¹H NMR (400 MHz, DMSO-d₆): δ 12.10 (br, 1H), 8.01 (d, 1H), 7.50 (m, 9H), 5.54 (s, 1H), 4.90 (m, 2H), 4.60 (s, 2H), 4.15 (m, 4H), 2.62 (q, 2H), 2.51 (s, 3H), 1.01 (t, 3H). MS (EI) for C₂₉H₂₈N₃O₅S, found 550 (MH+).

5-[4-(4-{[2-Ethyl-3-fluoro-4-(methylsulfonyl)phenyl]-carbonyl}-2,3,4,5-tetrahydro-1,4-benzoxazepin-7-yl)phenyl]-2,4-dihydro-3H-1,2,4-triazol-3-one. ¹H NMR (400 MHz, DMSO-d₆): δ 12.07 (s, 1H), 11.72-11.69 (m, 1H), 7.89-7.85 (m, 1H), 7.83-7.71 (m, 3.5H), 7.64-7.53 (m, 2H), 7.29 (d, 0.5H), 7.16-7.07 (m, 1.5H), 6.86 (d, 0.5H), 5.06-4.81 (m, 1H), 4.52-3.99 (m, 4H), 3.63-3.57 (m, 1H), 3.39-3.35 (m, 3H), 2.68-2.02 (m, 2H), 1.10 (t, 1.5H), 0.99 (t, 1.5H). MS (EI) for C₂₇H₂₅FN₄O₅S, found 537 (MH+).

4-{[2-Ethyl-3-fluoro-4-(methylsulfonyl)phenyl]carbonyl}-7-[4-(1-ethyl-1H-imidazol-4-yl)phenyl]-2,3,4,5-tetrahydro-1,4-benzoxazepine. ¹H NMR (400 MHz, DMSO-d₆): δ 7.87-7.26 (m, 9H), 7.19-6.76 (m, 2H), 5.07-4.78 (m, 1H), 4.50-3.98 (m, 6H), 3.67-3.55 (m, 1H), 3.38 (s, 3H), 2.71-2.02 (m, 2H), 1.43-1.37 (m, 3H), 1.20-0.96 (m, 3H). MS (EI) for C₃₀H₃₀FN₃O₄S, found 548 (MH+).

1-{5-[4-(4-{[2-Ethyl-3-fluoro-4-(methylsulfonyl)phenyl]-carbonyl}-2,3,4,5-tetrahydro-1,4-benzoxazepin-7-yl)phenyl]-1H-imidazol-2-yl}-N-methylmethanamine. ¹H NMR (400 MHz, DMSO-d₆): δ 12.10 (br, 1H), 7.50 (m, 9H), 6.60 (s, 1H), 4.40 (s, 2H), 4.20 (m, 6H), 3.30 (s, 3H), 3.26 (d, 3H), 2.40 (q, 2H), 1.25 (t, 3H). MS (EI) for C₃₀H₃₁FN₄O₄S, found 563 (MH+).

7-(1H-Benzimidazol-6-yl)-4-{[4-(but-3-en-1-ylsulfonyl)-2-ethyl-3-fluorophenyl]carbonyl}-2,3,4,5-tetrahydro-1,4-benzoxazepine. ¹H NMR (400 MHz, CDCl₃): δ 8.34 (s, 1H), 8.00-7.97 (m, 1H), 7.90-7.88 (m, 1H), 7.56-7.51 (m, 3H), 7.29 (m, 1H), 7.24-7.22 (m 1H), 7.18-7.16 (m, 1H), 6.41-6.40 (m, 1H), 5.83-5.73 (m, 1H), 5.16-5.11 (m, 2H), 4.50-4.43 (s, 1H), 4.39-4.34 (m, 2H), 4.15-4.10 (m, 2H), 4.00-3.95 (m, 1H), 3.55-3.50 (m, 2H), 2.71-2.64 (m, 1H), 2.56-2.48 (m, 1H), 2.39-2.32 (m, 1H), 2.00-1.94 (m, 1H), 1.07-1.03 (m, 3H). MS (EI) for C₂₉H₂₈FN₃O₄S, found 534 (MH+).

### Example 3

### [4-(Methylsulfonyl)phenyl]{7-[2-(propan-2-yl)-1H-benzimidazol-6-yl]-2,3-dihydro-1,4-benzoxazepin-4(5H)-yl}methanone

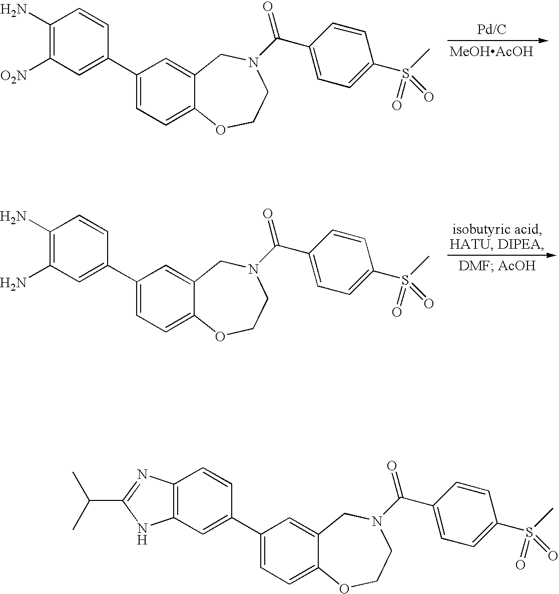

[7-(3,4-diaminophenyl)-2,3-dihydro-1,4-benzoxazepin-4(5H)-yl][4-(methylsulfonyl)phenyl]methanone. A mixture of 4-(4-{[4-(methylsulfonyl)phenyl]carbonyl}-2,3,4,5-tetrahydro-1,4-benzoxazepin-7-yl)-2-nitroaniline (196 mg, 0.42 mmol), prepared using the methods described in Example 2, and Pd/C (200 mg, 10% on carbon) in MeOH/AcOH (5 mL/1 mL) were reacted on the Parr hydrogenation for 2 h. The crude mixture was filtered on Celite and concentrated at reduced pressure. Purification by preparative HPLC afforded the desired product (151 mg, 82%). ¹H NMR (400 MHz, DMSO-d₆): δ 8.04-7.96 (dd, 2H), 7.66-7.48 (dd, 2H), 7.46-6.84 (m, 3H), 6.72-6.48 (m, 3H), 4.80-4.42 (d, 2H), 4.63-4.48 (m, 4H), 4.22-4.08 (m, 2H), 4.03-3.68 (m, 2H), 3.29-3.26 (d, 3H). MS (EI) for C₂₃H₂₃N₃O₄S: 437.9 (MH⁺).

7-[2-(1-methylethyl)-1H-benzimidazol-6-yl]-4-{[4-(methylsulfonyl)phenyl]carbonyl}-2,3,4,5-tetrahydro-1,4-benzoxazepine. To a stirred mixture of [7-(3,4-diaminophenyl)-2,3-dihydro-1,4-benzoxazepin-4(5H)-yl][4-(methylsulfonyl)phenyl]methanone (44 mg, 0.1 mmol), isobutyric acid (18.6 μL, 0.2 mmol), and DIPEA (87 μL, 0.5 mmol) in DMF (2 mL) was added HATU (38 mg, 0.1 mmol). After stirring at rt for 30 min, the reaction mixture was diluted with CH₂Cl₂, washed with aqueous 2 N NaOH, dried over Na₂SO₄, concentrated under reduced pressure. The residue was redissolved in AcOH (2 mL) and stirred at 70° C. for 1 h. The reaction mixture was cooled to rt and purification by preparative HPLC gave the desired product (17 mg, 35%). ¹H NMR (400 MHz, DMSO-d₆): δ 12.27 (bs, 1H), 8.00-7.98 (d, 2H), 7.82-7.51 (m, 6H), 7.36-6.84 (m, 2H), 4.89-4.55 (d, 2H), 4.31-4.16 (m, 2H), 4.06-3.71 (m, 2H), 3.31-3.26 (d, 3H), 2.53-2.51 (m, 1H), 1.43-1.39 (m, 6H). MS (EI) for C₂₇H₂₇N₃O₄S, found 490 (MH⁺).

### Example 4

### 4-(4-{[2-methyl-3-fluoro-4-(methylsulfonyl)-phenyl]carbonyl}-2,3,4,5-tetrahydro-1,4-benzoxazepin-7-yl)benzoic acid

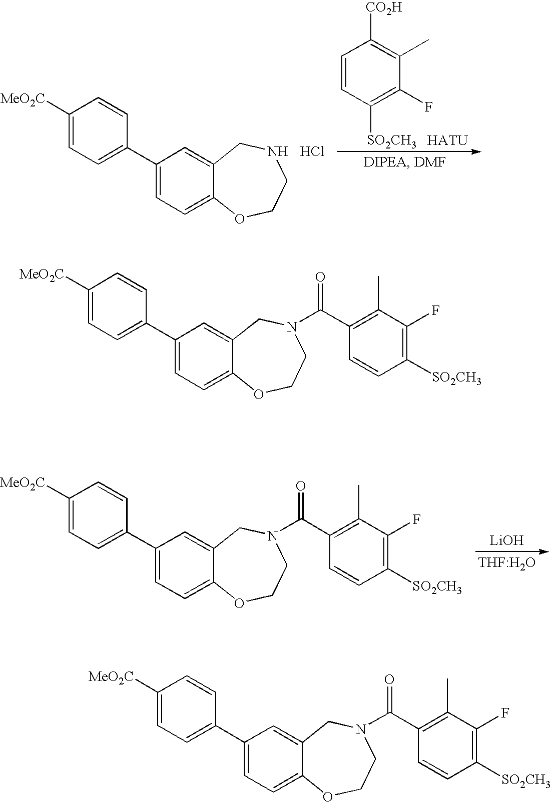

Methyl 4-(4-{[3-fluoro-2-methyl-4-(methylsulfonyl)phenyl]carbonyl}-2,3,4,5-tetrahydro-1,4-benzoxazepin-7-yl)benzoate. To the solution of ethyl 4-(2, 3, 4,5-tetrahydrobenzo[f][1,4]oxazepin-7-yl)benzoate hydrochloride (2.45 g, 7.35 mmol), prepared as described in Reference Example 2, in DMF (10 mL) was added 2-methyl-3-fluoro-4-(methylsulfonyl)benzoic acid (1.81 g, 7.35 mmol), HATU (2.79, 7.35 mmol) and DIPEA (1.90 g, 14.70 mmol). The reaction mixture was stirred at rt for 30 minutes and was partitioned with ethyl acetate (100 mL) and brine (100 mL). The aqueous layer was extracted with ethyl acetate (2×50 mL). The combined organic layer was dried with sodium sulfate. After evaporation of solvent, purification of the residue by silica gel chromatography gave ethyl 4-(4-{[2-ethyl-3-fluoro-4-(methylsulfonyl)phenyl]carbonyl}-2,3,4,5-tetrahydro-1,4-benzoxazepin-7-yl)benzoate (3.8 g, 98% yield). ¹H NMR (400 MHz, DMSO-d₆): δ 8.07-7.94 (m, 2H), 7.88-7.53 (m, 5H), 7.32-6.78 (m, 2H), 5.00-4.89 (m, 1H), 4.58-4.11 (m, 4H), 4.05-3.92 (m, 1H), 3.91-3.84 (m, 2H), 3.59 (m, 1H), 3.36 (s, 2H), 2.15-1.73 (m, 3H). MS (EI) for C₂₆H₂₄FNO₆S, found 498 (MH+).

4-(4-{[2-Methyl-3-fluoro-4-(methylsulfonyl)phenyl]carbonyl}-2,3,4,5-tetrahydro-1,4-benzoxazepin-7-yl)benzoic acid. To the solution of methyl 4-(4-{[2-methyl-3-fluoro-4-(methylsulfonyl)phenyl]carbonyl}-2,3,4,5-tetrahydro-1,4-benzoxazepin-7-yl)benzoate (3.8 g, 7.23 mmol) in THF (20 mL) was added lithium hydroxide (1.21 g, 28.9 mmol) at rt. The reaction mixture was heated to 60° C. for 3.5 h (monitored by LC/MS). The reaction mixture was to cooled to rt and neutralized with aqueous 3 N HCl to pH 1. The precipitate was collected by filtration, washed with water and dried under vacuum to give 4-(4-{[2-methyl-3-fluoro-4-(methylsulfonyl)phenyl]carbonyl}-2,3,4,5-tetrahydro-1,4-benzoxazepin-7-yl)benzoic acid, as a white powder (3.17 g, 88% yield). ¹H NMR (400 MHz, DMSO); δ 12.99 (s, 1H), 8.08-7.91 (m, 2H), 7.86-7.26 (m, 5H), 7.21-6.77 (m, 2H), 5.03-4.86 (m, 1H), 4.57-3.91 (m, 4H), 3.67-3.49 (m, 1H), 2.15-1.72 (m, 3H); MS (EI) for C₂₅H₂₂FNO₆S: 482.20 (M−H).

### Example 5

### (7-(4-(1H-Benzo[d]imidazol-2-yl)phenyl)-2,3-dihydrobenzo[f][1,4]oxazepin-4(5H)-yl)(2-ethyl-3-fluoro-4-(methylsulfonyl)phenyl)methanone

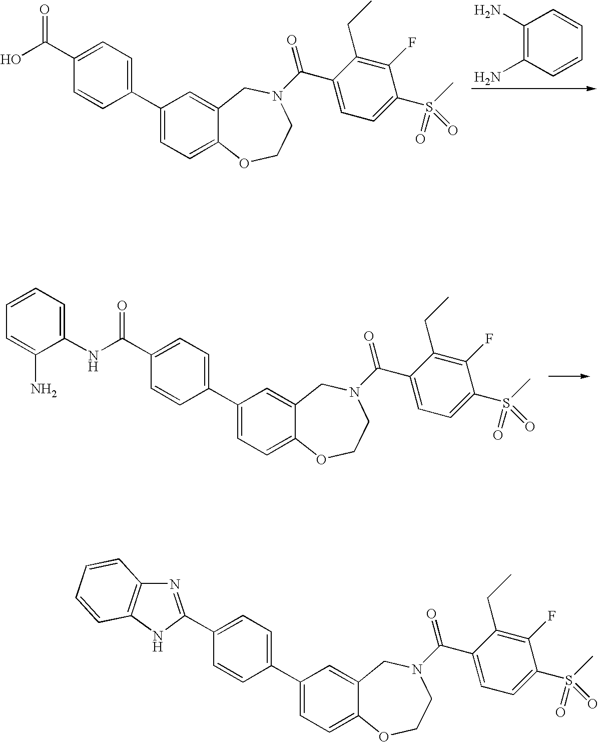

To a solution of 4-(4-(2-ethyl-3-fluoro-4-(methylsulfonyl)benzoyl)-2,3,4,5-tetrahydrobenzo[f][1,4]oxazepin-7-yl)benzoic acid (102 mg, 0.20 mmol), prepared as described in Example 4, and o-phenylenediamine (22 mg, 0.20 mmol) in DMF (5 mL) was added DIEA (110 μL, 0.61 mmol) and HATU (78 mg, 0.20 mmol). The reaction mixture was stirred at rt for 5 h. Solvent was removed at reduced pressure and purification of the residue by column chromatography gave N-(2-aminophenyl)-4-(4-(2-ethyl-3-fluoro-4-(methylsulfonyl)benzoyl)-2,3,4,5-tetrahydrobenzo[f][1,4]oxazepin-7-yl)benzamide (62 mg, 52%).

To a solution of N-(2-aminophenyl)-4-(4-(2-ethyl-3-fluoro-4-(methylsulfonyl)benzoyl)-2,3,4,5-tetrahydrobenzo[f][1,4]oxazepin-7-yl)benzamide (62 mg, 0.11 mmol) in acetic acid (5 mL) was stirred at 70° C. for 1 h. The resulting mixture was purified by preparative HPLC to give (7-(4-(1H-benzo[d]imidazol-2-yl)phenyl)-2,3-dihydrobenzo[f][1,4]oxazepin-4(5H)-yl)(2-ethyl-3-fluoro-4-(methylsulfonyl)phenyl)methanone (32 mg, 60%). ¹H NMR (400 MHz, DMSO-d₆): δ 12.99 (s, 1H), 8.24 (m, 2H), 7.89-7.64 (m, 7H), 7.30-6.91 (m, 4H), 5.05-4.88 (m, 1H), 4.47-4.07 (m, 4H), 3.60 (m, 1H), 3.40 (m, 3H), 2.66 (m, 1H), 2.36-2.08 (m, 1H), 1.10-1.00 (m, 3H). MS (EI) for C₃₂H₂₈FN₃O₄S, found 570 (MH+).

### Example 6

### N-[5-(4-{[2-Ethyl-4-(ethylsulfonyl)-3-fluorophenyl]carbonyl}-2,3,4,5-tetrahydro-1,4-benzoxazepin-7-yl)pyridin-2-yl]-N²-methylglycinamide

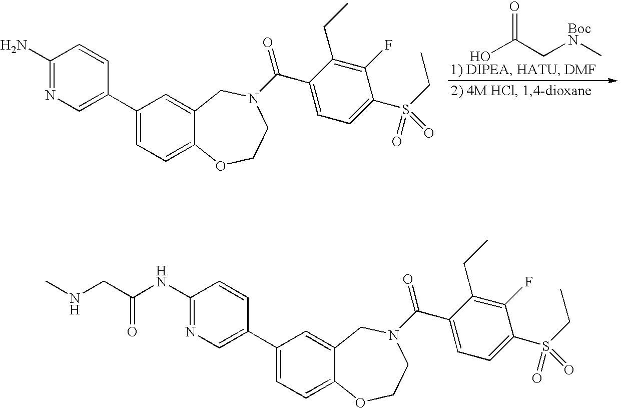

To a stirred solution of (7-(6-aminopyridin-3-yl)-2,3-dihydrobenzo[f][1,4]oxazepin-4(5H)-yl)(2-ethyl-4-(ethylsulfonyl)-3-fluorophenyl)methanone (78 mg, 0.16 mmol), prepared using procedures described in Example 2,2-(tert-butoxycarbonyl(methyl)amino)acetic acid (61 mg, 0.32 mmol), and DIPEA (111 mL, 0.64 mmol) in DMF (3 mL) was added HATU and the reaction was stirred at rt for 2 h. Purification of the crude mixture by preparative HPLC gave tert-butyl (2-{[5-(4-{[2-ethyl-4-(ethylsulfonyl)-3-fluorophenyl]carbonyl}-2,3,4,5-tetrahydro-1,4-benzoxazepin-7-yl)pyridin-2-yl]amino}-2-oxoethyl)methylcarbamate, which was dissolved in 1,4-dioxane (3 mL) and treated with 4 M HCl in 1,4-dioxane (2 mL). After stirring the mixture for 1 h, volatiles were removed at reduced pressure, and the residue was diluted with H₂O, basified with aqueous NaHCO₃, and extracted with CH₂Cl₂ (3×50 mL). The organic extracts were combined, dried over Na₂SO₄, and concentrated at reduced pressure to give N-[5-(4-{[2-ethyl-4-(ethylsulfonyl)-3-fluorophenyl]carbonyl}-2,3,4,5-tetrahydro-1,4-benzoxazepin-7-yl)pyridin-2-yl]-N²-methylglycinamide (18 mg, 20% over 2 steps). ¹H NMR (400 MHz, DMSO-d₆): δ 8.66-8.41 (dd, 1H), 8.21-8.11 (m, 1.5H), 7.86-7.83 (dd, 0.5H), 7.76-7.75 (dd, 0.5H), 7.73-7.69 (m, 1H), 7.60-7.54 (m, 1H), 7.30-7.28 (d, 0.5H), 7.11-7.07 (m, 1.5H), 6.80-6.80 (d, 0.5H), 5.03-4.27 (m, 2H), 4.45 (s, 1H), 4.18-3.57 (m, 4H), 3.37-3.31 (m, 3H), 3.32-3.31 (d, 2H), 2.68-2.10 (m, 2H), 2.34-2.33 (d, 3H), 1.18-0.98 (m, 6H). MS (EI) for C₂₈H₃ᵢFN₄O₅S: 555.2 (MH⁺).

### Example 7

### 5-(4-{[2-ethyl-3-fluoro-4-(methylsulfonyl)phenyl]carbonyl}-2,3,4,5-tetrahydro-1,4-benzoxazepin-7-yl)-N-(2-pyrrolidin-1-ylethyl)pyridin-2-amine

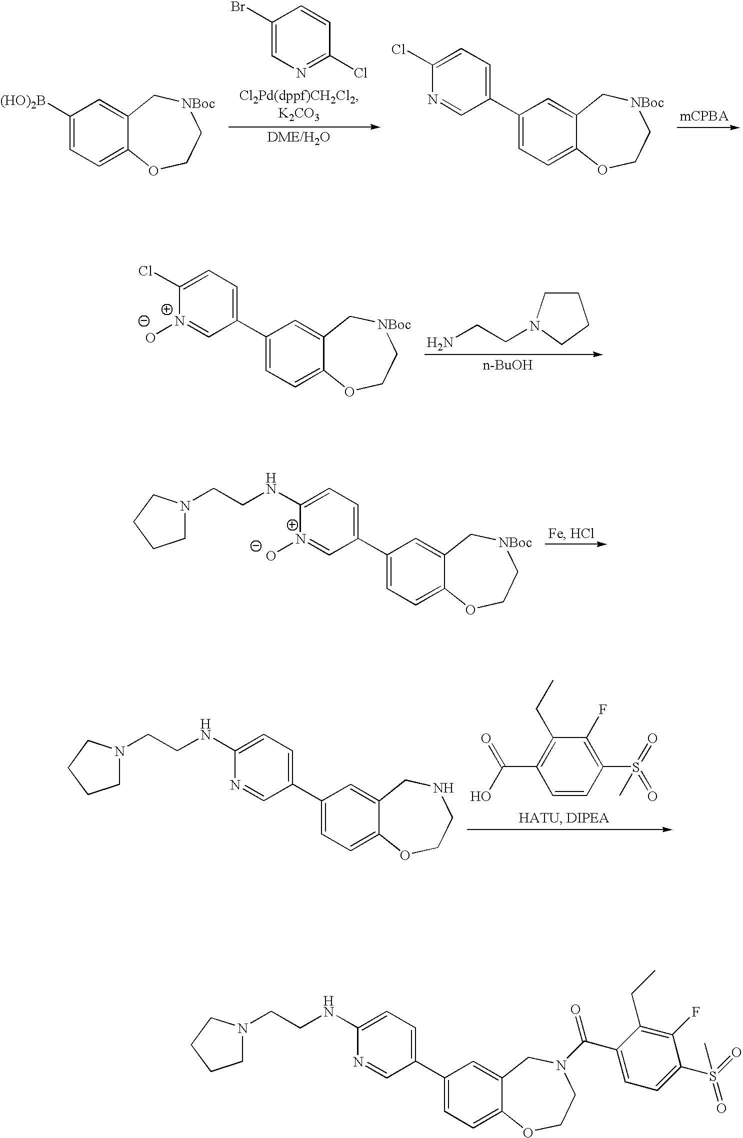

To a stirred suspension of 5-bromo-2-chloropyridine (3.84 g, 20 mmol), 7-diboronic acid-2,3-dihydro-5H-benzo[f][1,4]-oxazepine-4-carboxylic acid tert-butyl ester (4.98, 20 mmol), prepared as described in Reference Example 5, and K₂CO₃ (8.29 g, 60 mmol) in DME/H₂O (90 mL/10 mL) was added Cl₂Pd(dppf)CH₂Cl₂ (1.63 g, 2.0 mmol). After stirring for 12 h at 75° C., the reaction mixture was cooled to rt, concentrated under reduced pressure. Purification of the residue by flash chromatography gave tert-butyl 7-(6-chloropyridin-3-yl)-2,3-dihydro-1,4-benzoxazepine-4(5H)-carboxylate (4.8 g, 67%).

To the tert-butyl 7-(6-chloropyridin-3-yl)-2,3-dihydro-1,4-benzoxazepine-4(5H)-carboxylate (3.6 g, 10 mmol) in chloroform (50 mL) was added m-CPBA (3.37 g, 15 mmol). The reaction mixture was heated to 70° C., for 12 h. The reaction was quenched by addition of aqueous 2 M NaOH, and the separated organic layer was dried over Na₂SO₄. Evaporation of the solvent in vacuo, followed by flash column chromatography (30% hexane/EtOAc) provided the 5-(4-(tert-butoxycarbonyl)-2,3,4,5-tetrahydrobenzo[f][1,4]oxazepin-7-yl)-2-chloropyridine 1-oxide (3.2 g, 85%). MS (EI) for C₁₉H₂₁ClN₂O₄, found 377 (MH+).

To the 5-(4-(tert-butoxycarbonyl)-2,3,4,5-tetrahydrobenzo[f][1,4]oxazepin-7-yl)-2-chloropyridine 1-oxide (376 mg, 1 mmol) in n-BuOH (2 mL) was added 2-(pyrrolidin-1-yl)ethanamine (5 equiv), and the reaction mixture was heated at 160° C. for 1 h. The reaction mixture was cooled to rt, diluted with methylene chloride, washed with brine, and concentrated in vacuo. The residue was treated with iron (110 mg, 2.0 mmol) in HCl (2 mL), diluted with methylene chloride, washed with aqueous NaHCO₃, and the separated aqueous layer was extracted with methylene chloride. The organic extract was dried over Na₂SO₄, concentrated under reduced pressure, and 5-(4-(tert-butoxycarbonyl)-2,3,4,5-tetrahydrobenzo[f][1,4]oxazepin-7-yl)-2-(2-(pyrrolidin-1-yl)ethylamino)pyridine 1-oxide (200 mg, 53.2%) was used in the next step without further purification.

To a stirred solution of 5-(4-(tert-butoxycarbonyl)-2,3,4,5-tetrahydrobenzo[f][1,4]oxazepin-7-yl)-2-(2-(pyrrolidin-1-yl)ethylamino)pyridine 1-oxide (150 mg, 0.34 mmol), 2-ethyl-3-fluoro-4-(methylsulfonyl)benzoic acid (82.9 mg, 0.34 mmol), prepared as described in Reference Example 3, and DIPEA (0.4 mL) in DMF was added HATU (128 mg, 0.34 mmol). After stirring for 10 min at 50° C., the reaction mixture was purified by preparative HPLC to provide 5-(4-{[2-ethyl-3-fluoro-4-(methylsulfonyl)phenyl]carbonyl}-2,3,4,5-tetrahydro-1,4-benzoxazepin-7-yl)-N-(2-pyrrolidin-1-ylethyl)pyridin-2-amine (100 mg, 51.6%). ¹H NMR (400 MHz, DMSO-d₆): δ 8.38-6.58 (m, 8H), 4.81 (m, 1H), 4.44-3.98 (m, 5H), 3.58 (m, 4H), 3.4 (s, 3H), 3.2 (m, 4H), 2.47-1.98 (m, 2H), 1.09 and 0.97 (t, 3H). MS (EI) for C₃₀H₃₅FN₄O₄S, found 567 (MH+).

### Example 8

### 1-(4-{[7-(1-Methyl-1H-indazol-6-yl)-2,3-dihydro-1,4-benzoxazepin-4(5H)-yl]carbonyl}phenyl)ethanone

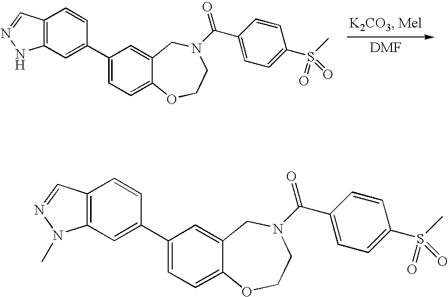

To a stirred solution of 1-(4-{[7-(1H-indazol-6-yl)-2,3-dihydro-1,4-benzoxazepin-4(5H)-yl]carbonyl}-phenyl)ethanone (41 mg, 0.1 mmol), prepared using procedures as described in Example 2, in DMF (2 mL) was added K₂CO₃ (42 mg, 0.3 mmol) and MeI (100 μL). The mixture was to stirred at rt overnight. Purification of the crude mixture by preparative HPLC gave 1-(4-{[7-(1-methyl-1H-indazol-6-yl)-2,3-dihydro-1,4-benzoxazepin-4 (5H)-yl]carbonyl}phenyl)ethanone (24 mg, 56%). ¹H NMR (400 MHz, DMSO-d₆): δ 8.06-7.81 (m, 5H), 7.78-7.53 (m, 3H), 7.41-6.97 (m, 3H), 4.91-4.55 (d, 2H), 4.32-4.17 (m, 2H), 4.12-4.07 (d, 3H), 4.05-3.74 (m, 2H), 2.61 (s, 3H). MS (EI) for C₂₆H₂₃N₃O₃, 426.0 (MH⁺).

### Example 10

### [7-(5-Amino-1,3,4-thiadiazol-2-yl)-2,3-dihydro-1,4-benzoxazepin-4(5H)-yl][2-ethyl-3-fluoro-4-(methylsulfonyl)phenyl]methanone

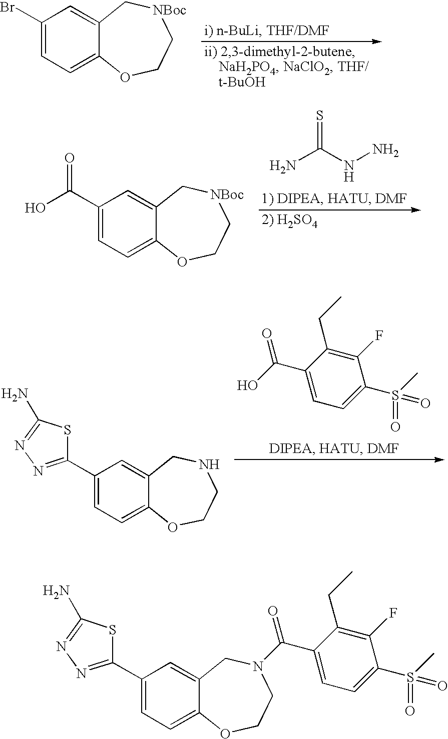

To a stirred solution of tert-butyl-7-bromo-2,3-dihydrobenzo[f][1,4]oxazepine-4(5H)-carboxylate (6.56 g, 20 mmol), prepared as described in Reference Example 4, in THF (50 mL) at −78° C. was added n-BuLi (8 mL, 2.5 M in Hexanes) dropwise and the mixture was stirred at −78° C. for 1 h. To this mixture at −78° C. was added DMF (2.32 mL, 30 mmol) and the resulting mixture was stirred at −78° C. for 2 h. The cooling bath was removed and the reaction mixture was warmed to rt, quenched by adding H₂O, and extracted with CH₂Cl₂ (2×100 mL). The extracts were dried over Na₂SO₄, concentrated under reduced pressure, and purification of the residue by flash chromatography gave tert-butyl 7-formyl-2,3-dihydrobenzo[f][1,4]oxazepine-4(5H)-carboxylate (4.81 g, 87%).

To a stirred solution of tert-butyl 7-formyl-2,3-dihydrobenzo[f][1,4]oxazepine-4(5H)-carboxylate (2.77 g, 10 mmol) in THF/t-BuOH (20 mL/5 mL) was added 2,3-dimethyl-2-butene (3.57 mL, 30 mmol) and NaH₂PO₄ (1.2 g in 10 mL of H₂O). To this mixture was added NaClO₂ (2.26 g, 20 mmol) portionwise. After stirring for 1 h, the reaction mixture was diluted with H₂O, neutralized to pH 6-7, and extracted with CH₂Cl₂ (3×100 mL). The extracts were dried over Na₂SO₄, concentrated under reduced pressure, and the residue was triturated with Hexanes to give 4-(tert-butoxycarbonyl)-2,3,4,5-tetrahydrobenzo[f][1,4]oxazepine-7-carboxylic acid (2.84 g, 97%).

To a stirred solution of 4-(tert-butoxycarbonyl)-2,3,4,5-tetrahydrobenzo[f][1,4]oxazepine-7-carboxylic acid (293 mg, 1.0 mmol), thiosemicarbazide (91 mg, 1.0 mmol), and DIPEA (523 μL, 3.0 mmol) in DMF (4 mL) was added HATU (380 mg, 1.0 mmol). After stirring at rt for 10 min, the reaction mixture was diluted with CH₂Cl₂, washed with aqueous NaHCO₃, and the separated aqueous layer was extracted with CH₂Cl₂ (2×50 mL). The combined organic layers were dried over Na₂SO₄, concentrated under reduced pressure, and the residue was taken in conc. H₂SO₄ (2 mL). After stirring at 100° C. for 1 h, the reaction mixture was cooled to rt, poured into ice, adjusted to pH 8-9 with 1 M NaOH and extracted with CH₂Cl₂ (6×50 mL). The combined organic extracts were dried over Na₂SO₄, concentrated under reduced pressure to give 5-(2,3,4,5-tetrahydrobenzo[f][1,4]oxazepin-7-yl)-1,3,4-thiadiazol-2-amine, which was then reacted with ethyl 4-(2,3,4,5-tetrahydro-1,4-benzoxazepin-7-yl)benzoate, prepared as described in Reference Example 3, under HATU coupling conditions to give [7-(5-amino-1,3,4-thiadiazol-2-yl)-2,3-dihydro-1,4-benzoxazepin-4(5H)-yl][2-ethyl-3-fluoro-4-(methylsulfonyl)phenyl]methanone (77 mg, 16% over 3 steps). ¹H NMR (400 MHz, DMSO-d₆): δ 7.76-7.70 (m, 1.5H), 7.62-7.57 (m, 1H), 7.39-7.38 (d, 2H), 7.29-7.08 (dd, 1H), 7.08-7.05 (dd, 1H), 6.91-6.91 (dd, 0.5H), 5.00-4.40 (m, 2H), 4.36-3.56 (m, 4H), 3.40-3.35 (d, 3H), 2.67-2.06 (m, 2H), 1.10-0.98 (m, 3H). MS (EI) for C₂₁H₂₁FN₄O₄S₂, 477.0 (MH⁺).

Using the same or analogous synthetic techniques and substituting with appropriate reagents, 5-(4-{[2-Ethyl-4-(ethylsulfonyl)-3-fluorophenyl]carbonyl}-2,3,4,5-tetrahydro-1,4-benzoxazepin-7-yl)-1,3,4-thiadiazol-2-amine was prepared. ¹H NMR (400 MHz, CDCl₃): δ 7.88-6.58 (m, 5H), 5.39 (m, 2H), 4.87 (m, 1H), 4.49-4.30 (m, 7H), 2.80-2.17 (m, 2H), 1.37 (m, 3H), 1.18 (m, 3H). MS (EI) for C₂₂H₂₃FN₄O₄S₂, found 491 (MH+).

### Example 11

### 2-Ethyl-5-methoxy-4-(methylsulfonyl)phenyl][7-(2-methyl-1H-benzimidazol-6-yl)-2,3-dihydro-1,4-benzoxazepin-4(5H)-yl]methanone; and [3-Methoxy-4-(methylsulfonyl)phenyl][7-(2-methyl-1H-benzimidazol-6-yl)-2,3-dihydro-1,4-benzoxazepin-4(5H)-yl]methanone

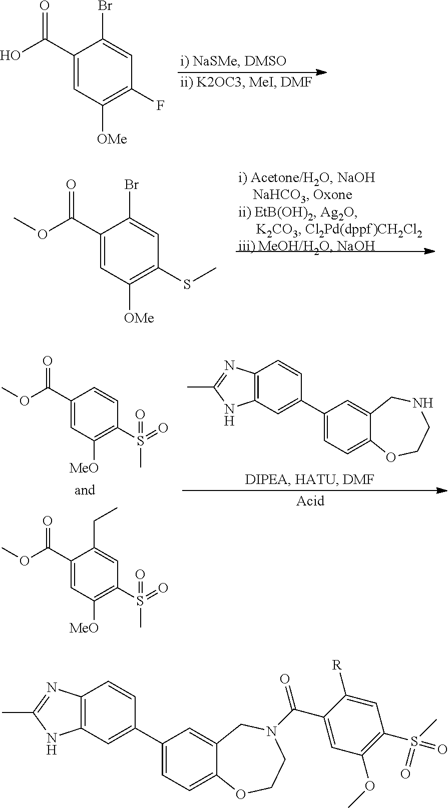

To a stirred solution of 2-bromo-4-fluoro-5-methoxybenzoic acid (249 mg, 1.0 mmol), prepared as described above in Reference Example 7, in DMSO (4 mL) was added NaSMe (210 mg, 3.0 mmol) and the mixture was stirred at 100° C. for 1 h. 2-bromo-5-hydroxy-4-thiomethylbenzoic acid was obtained as a major product. The reaction mixture was diluted with H₂O, acidified with 1 N HCl to pH 1-2, and extracted with CH₂Cl₂ (3×50 mL). The combined extracts were dried over Na₂SO₄, concentrated under reduced pressure, and the residue was taken in DMF (4 mL) and treated with K₂CO₃ (415 mg, 3.0 mmol) and MeI (374 μL, 6.0 mmol). After stirring at rt for 1 h, the reaction mixture was diluted with EtOAc/H₂O and the separated aqueous layer was extracted with EtOAc. The combined extracts were dried over Na₂SO₄, concentrated under reduced pressure, and the residue was purified by flash chromatography to afford methyl 2-bromo-5-methoxy-4-(methylthio)benzoate (227 mg, 78% over 2 steps).

To a stirred solution of methyl 2-bromo-5-methoxy-4-(methylthio)benzoate (227 mg, 0.78 mmol) in acetone/H₂O (4 mL/4 mL) was added 1 M NaOH (0.78 mL), NaHCO₃ (197 mg, 2.4 mmol), and oxone (2 g). The reaction mixture was stirred at rt for 30 min, diluted with EtOAc/H₂O, and the separated aqueous layer was extracted with EtOAc. The combined organic layers were dried over Na₂SO₄, concentrated under reduced pressure, and the residue was purified by flash chromatography to give methyl 2-bromo-5-methoxy-4-(methylsulfonyl)benzoate (226 mg, 90%).

To a stirred solution of methyl 2-bromo-5-methoxy-4-(methylsulfonyl)benzoate (97 mg, 0.3 mmol) in THF (6 mL) was added EtB(OH)₂ (25 mg, 0.34 mmol), Ag₂O (174 mg, 0.75 mmol), K₂CO₃ (125 mg, 0.90 mmol), and Cl₂Pd(dppf)CH₂Cl₂ (37 mg, 0.045 mmol). The mixture was stirred for 16 h at reflux, cooled to rt, concentrated under reduced pressure, and the residue was purified by flash chromatography to give methyl 3-methoxy-4-(methylsulfonyl)benzoate (“reduced intermediate”) and methyl 2-ethyl-5-methoxy-4-(methylsulfonyl)benzoate (“ethyl intermediate”) in an inseparable mixture (34 mg).

To a stirred solution of a mixture of the reduced intermediate and the ethyl intermediate mixture (34 mg) in MeOH (6 mL) was added 1 M NaOH (1 mL) and the mixture was stirred for 3 h. MeOH was removed at reduced pressure and the residue was diluted with H₂O, acidified with 1 N HCl to pH 1-2, and extracted with CH₂Cl₂ (4×50 mL). The extracts were dried over Na₂SO₄, concentrated under reduced pressure, and the mixture of 3-methoxy-4-(methylsulfonyl)benzoic acid and 2-ethyl-5-methoxy-4-(methylsulfonyl)benzoic acid (30 mg, 40% of reduced intermediate and 60% ethyl intermediate) was used for the next step without further purification.

7-(2-Methyl-1H-benzimidazol-6-yl)-2,3,4,5-tetrahydro-1,4-benzoxazepine, prepared using procedures described in Reference Example 6, was treated with the mixture of 3-methoxy-4-(methylsulfonyl)benzoic acid and 2-ethyl-5-methoxy-4-(methylsulfonyl)benzoic acid using standard HATU conditions to yield 2-ethyl-5-methoxy-4-(methylsulfonyl)phenyl][7-(2-methyl-1H-benzimidazol-6-yl)-2,3-dihydro-1,4-benzoxazepin-4(5H)-yl]methanone [¹H NMR (400 MHz, DMSO-d₆): δ 12.25-12.13 (m, 1H), 7.70-7.40 (m, 5H), 7.14-7.06 (m, 2H), 6.76-6.72 (m, 1H), 5.04-4.55 (m, 1H), 4.30-3.91 (m, 5H), 3.91-3.58 (d, 3H), 3.25-3.20 (d, 3H), 2.49 (d, 3H), 2.49-2.24 (m, 2H), 1.11-1.00 (d, 3H). MS (EI) for C₂₈H₂₉N₃O₅S: 519.9 (MH⁺)] as well as [3-methoxy-4-(methylsulfonyl)phenyl][7-(2-methyl-1H-benzimidazol-6-yl)-2,3-dihydro-1,4-benzoxazepin-4(5H)-yl]methanone [¹H NMR (400 MHz, CDCl₃): δ 12.23-12.15 (m, 1H), 7.84-7.80 (m, 1H), 7.71-7.17 (m, 5H), 7.13-6.92 (m, 3H), 4.83-4.51 (d, 2H), 4.30-4.11 (m, 2H), 4.02-3.70 (m, 2H), 3.94-3.61 (d, 3H), 3.24-3.19 (m, 3H), 2.49 (s, 3H). MS (EI) for C₂₆H₂₅N₃O₅S: 491.9 (MH⁺)].

### Example 12

### 5-(4{[2-Ethyl-3-fluoro-4-(methylsulfonyl)phenyl]carbonyl}-2,3,4,5-tetrahydro-1,4-benzoxazepin-7-yl)-1,3-thiazol-2-amine

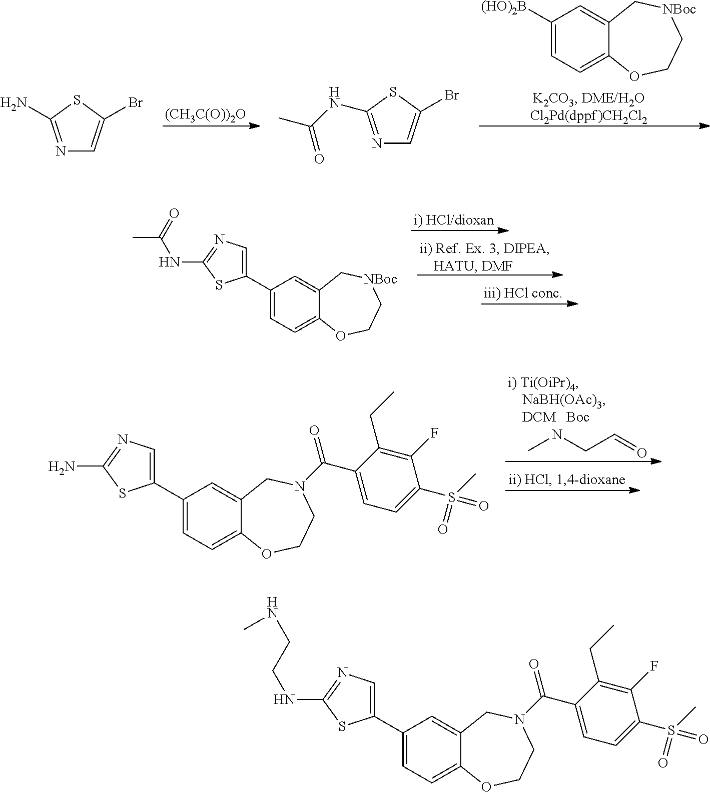

A mixture of commercially-available 2-amino-5-bromothiazole (5.37 g, 30 mmol) and acetic anhydride (25 mL) was heated at reflux for 30 min. The mixture was cooled to rt and diluted with water. The resulting solid, N-(5-bromothiazol-2-yl)acetamide, was collected by filtration, washed with water, dried in vacuo and taken to the next step without further purification (6.0 g, 90%). MS (EI) for C₅H₅BN₂OS, found 221.2 (MH+).

To a stirred mixture of N-(5-bromothiazol-2-yl)acetamide (6.0 g, 27 mmol), 7-diboronic acidic-2,3-dihydro-5H-benzo[f][1,4]-oxazepine-4-carboxylic acid tert-butyl ester (10.0 g, 34 mmol), prepared as described in Reference Example 4, and K₂CO₃ (46.8 g, 34.0 mmol) in DME/H₂O (200 mL/20 mL) was added Cl₂Pd(dppf)CH₂Cl₂ (2.14 g, 2.7 mmol). The mixture was stirred at reflux overnight. The reaction mixture was cooled to rt, concentrated at reduced pressure, and the residue was purified by flash chromatography to afford tert-butyl 7-[2-(acetylamino)-1,3-thiazol-5-yl]-2,3-dihydro-1,4-benzoxazepine-4(5H)-carboxylate (6.0 g, 57%). MS (EI) for C₁₉H₂₃N₃O₄S, found 389 (MH+).

To a stirred suspension of tert-butyl 7-[2-(acetylamino)-1,3-thiazol-5-yl]-2,3-dihydro-1,4-benzoxazepine-4(5H)-carboxylate (6.0 g, 15.4 mmol) in 1,4-dioxane (50 mL) was added 4 N hydrogen chloride in 1,4-dioxane (10 mL) and the reaction mixture was stirred at rt overnight. 1,4-Dioxane was removed under reduced pressure and the residue was triturated with Et₂O (2×50 mL). N-[5-(2,3,4,5-tetrahydro-1,4-benzoxazepin-7-yl)-1,3-thiazol-2-yl]acetamide (5.0 g, quant.) was isolated by filtration as the HCl salt. MS (EI) for C₁₄H₁₅N₃O₂S(HCl salt), found 289 (MH+).

To a mixture of 2-ethyl-3-fluoro-4-(methylsulfonyl)benzoic acid (2.53 g, 10.3 mmol), prepared as described in Reference Example 3, DIPEA (1.75 mL, 10.3 mmol), and HATU (4.04 g, 10.6 mmol) in DMF (1.0 mL) was added a solution of N-[5-(2,3,4,5-tetrahydro-1,4-benzoxazepin-7-yl)-1,3-thiazol-2-yl]acetamide hydrochloride salt (5.0 g, 10.3 mmol) and DIEA (5.2 mL, 30 mmol) in DMF (20.0 mL). The resulting reaction mixture was stirred at 50° C. for 30 min, then diluted with aqueous 5% NaHCO₃. The resulting solids were filtered, washed with H₂O and dried. The resulting N-[5-(4-{[2-Ethyl-3-fluoro-4-(methylsulfonyl)-phenyl]carbonyl}-2,3,4,5-tetrahydro-1,4-benzoxazepin-7-yl)-1,3-thiazol-2-yl]acetamide was purified by silica column chromatography. ¹H NMR (400 MHz, DMSO-d₆): δ 12.28 (s, 1H), 7.81-6.73 (m, 6H), 4.94 (m, 1H), 4.43-3.98 (m, 4H), 3.59 (s, 1H), 3.32 (s, 3H), 2.74-1.98 (m, 2H), 2.14 (s, 3H), 1.17 and 0.98 (t, 3H). MS (EI) for C₂₄H₂₄FN₃O₅S₂, found 518 (MH+).

N-[5-(4-{[2-Ethyl-3-fluoro-4-(methylsulfonyl)-phenyl]carbonyl}-2,3,4,5-tetrahydro-1,4-benzoxazepin-7-yl)-1,3-thiazol-2-yl]acetamide was treated with conc. hydrochloric acid (10 mL) in methanol (50 mL) and the mixture heated at 70° C. for 30 min. The solvent was removed and the residue was neutralized with aqueous 1 M sodium hydroxide. The filter cake was dried and washed with methanol and dried to yield 5-(4{[2-ethyl-3-fluoro-4-(methylsulfonyl)phenyl]carbonyl}-2,3,4,5-tetrahydro-1,4-benzoxazepin-7-yl)-1,3-thiazol-2-amine (2.56 g, 52.3%).

The mixture of 5-(4{[2-ethyl-3-fluoro-4-(methylsulfonyl)phenyl]carbonyl}-2,3,4,5-tetrahydro-1,4-benzoxazepin-7-yl)-1,3-thiazol-2-amine (71 mg, 0.15 mmol), aldehyde (78 mg, 0.45 mmol), and Ti(OiPr)₄ (67 μL, 0.23 mmol) in CH₂Cl₂ (7 mL) were stirred for 30 min. To this reaction mixture was added NaBH(OAc)₃. After stirring at rt for 5 h, the reaction mixture was diluted with aqueous NaHCO₃/CH₂Cl₂ and the separated aqueous layer was extracted with CH₂Cl₂. The combined extracts were dried over Na₂SO₄, concentrated under reduced pressure, and the residue was purified by preparative HPLC to give tert-butyl 2-(5-(4-(2-ethyl-3-fluoro-4-(methylsulfonyl)benzoyl)-2,3,4,5-tetrahydrobenzo[f][1,4]oxazepin-7-yl)thiazol-2-ylamino)ethyl(methyl)carbamate (61 mg, 64%).

To a stirred solution of tert-butyl 2-(5-(4-(2-ethyl-3-fluoro-4-(methylsulfonyl)benzoyl)-2,3,4,5-tetrahydrobenzo[f][1,4]oxazepin-7-yl)thiazol-2-ylamino)ethyl(methyl)carbamate (61 mg, 0.096 mmol) in 1,4-dioxane (4 mL) was added HCl (1 mL, 4 M HCl in 1,4-dioxane) and the mixture was stirred at rt for 6 h. The reaction mixture was concentrated under reduced pressure, basified with aqueous NaHCO₃, and extracted with CH₂Cl₂ (3×50 mL). The combined extracts were dried over Na₂SO₄, concentrated under reduced procedure to afford [2-ethyl-3-fluoro-4-(methylsulfonyl)phenyl][7-(2-{[2-(methylamino)ethyl]amino}-1,3-thiazol-5-yl)-2,3-dihydro-1,4-benzoxazepin-4(5H)-yl]methanone (26 mg, 51%).

¹H NMR (400 MHz, DMSO-d₆): δ 7.78-7.67 (m, 2H), 7.46-7.38 (m, 0.5H), 7.41 (s, 0.5H), 7.30-7.26 (m, 1.5H), 7.20 (s, 0.5H), 7.11-7.06 (dd, 0.5H), 7.00-6.95 (m, 1H), 6.54-6.47 (dd, 0.5H), 4.95-4.33 (m, 2H), 4.23-3.34 (m, 4H), 3.39-3.35 (d, 3H), 2.70-2.65 (q, 2H), 2.67-2.07 (m, 2H), 2.31-2.29 (m, 3H), 1.43-1.41 (d, 2H), 1.11-0.99 (m, 3H). MS (EI) for C₂₅H₂₉FN₄O₄S₂: 533.2 (MH⁺).

Using the same or analogous synthetic techniques and substituting with appropriate reagents, the following examples were prepared.

[2-Ethyl-3-fluoro-4-(methylsulfonyl)phenyl][7-(2-{[(25)-pyrrolidin-2-ylmethyl]amino}-1,3-thiazol-5-yl)-2,3-dihydro-1,4-benzoxazepin-4(5H)-yl]methanone. ¹H NMR (400 MHz, DMSO-d₆): δ 7.83-7.71 (m, 2H), 7.41-7.41 (d, 0.5H), 7.40 (s, 0.5H), 7.29-7.25 (m, 1.5H), 7.19 (s, 0.5H), 7.11-7.09 (d, 0.5H), 6.98-6.95 (dd, 1H), 6.54-6.53 (dd, 0.5H), 4.95-4.34 (m, 2H), 4.24-3.54 (m, 4H), 3.39-3.35 (d, 3H), 3.29-2.74 (m, 4H), 2.67-2.05 (m, 2H), 1.84-1.31 (m, 4H), 1.11-0.99 (m, 3H). MS (EI) for C₂₇H₃ᵢFN₄O₄S₂: 559.2 (MH⁺).

[2-Ethyl-3-fluoro-4-(methylsulfonyl)phenyl][7-(2-{[(2R)-pyrrolidin-2-ylmethyl]amino}-1,3-thiazol-5-yl)-2,3-dihydro-1,4-benzoxazepin-4(5H)-yl]methanone. ¹H NMR (400 MHz, DMSO-d₆): δ 7.83-7.71 (m, 2H), 7.41-7.41 (d, 0.5H), 7.40 (s, 0.5H), 7.29-7.25 (m, 1.5H), 7.19 (s, 0.5H), 7.11-7.09 (d, 0.5H), 6.98-6.95 (dd, 1H), 6.54-6.53 (dd, 0.5H), 4.95-4.34 (m, 2H), 4.24-3.54 (m, 4H), 3.39-3.35 (d, 3H), 3.29-2.74 (m, 4H), 2.67-2.05 (m, 2H), 1.84-1.31 (m, 4H), 1.11-0.99 (m, 3H). MS (EI) for C₂₇H₃ᵢFN₄O₄S₂: 559.2 (MH⁺).

[2-Ethyl-3-fluoro-4-(methylsulfonyl)phenyl][7-(6-{[2-(methylamino)ethyl]amino}pyridin-3-yl)-2,3-dihydro-1,4-benzoxazepin-4(5H)-yl]methanone. ¹H NMR (400 MHz, DMSO-d₆): δ 8.29-8.28 (d, 0.5H), 8.09-8.08 (d, 0.5H), 7.78-7.67 (m, 1.5H), 7.57-7.56 (d, 0.5H), 7.44-7.39 (m, 1.5H), 7.28-7.26 (d, 0.5H), 7.17-7.15 (d, 0.5H), 7.04-7.01 (dd, 1H), 6.64-6.65 (d, 0.5H), 6.60-6.55 (m, 1.5H), 6.50-6.48 (d, 0.5H), 5.00-4.34 (m, 2H), 4.29-3.55 (m, 4H), 3.39-3.35 (d, 3H), 3.34-3.29 (m, 2H), 2.67-2.62 (q, 2H), 2.30-2.30 (d, 3H), 2.67-2.00 (m, 2H), 1.11-0.95 (m, 3H). MS (EI) for C₂₇H₃₁FN₄O₄S: 527.2 (MH⁺).

### Example 13

### [7-(2-Amino-1,3-oxazol-5-yl)-2,3-dihydro-1,4-benzoxazepin-4(5H)-yl][2-ethyl-3-fluoro-4-(methylsulfonyl)phenyl]methanone

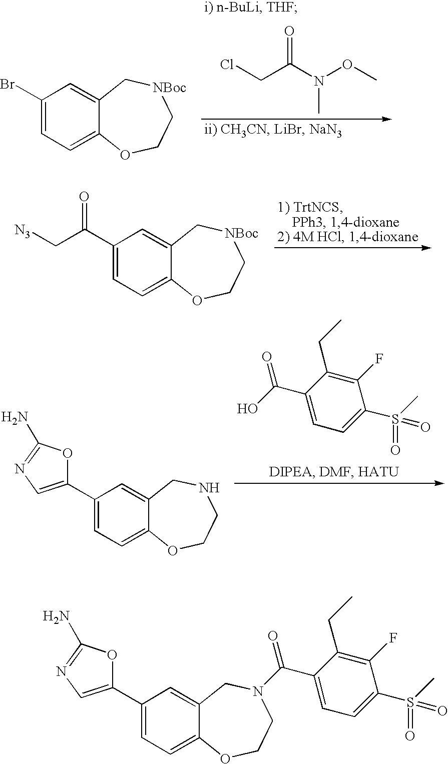

To a stirred solution of tert-butyl 7-bromo-2,3-dihydrobenzo[f][1,4]oxazepine-4(5H)-carboxylate (5.8 g, 17.7 mmol), prepared as described in Reference Example 4, in THF (35 mL) was added n-BuLi (7 mL, 2.5 M in hexanes) dropwise at −78° C. and the mixture was stirred at −78° C. for 1 h. To this mixture was added Weinreb amide (2.64 g, 19.2 mmol) in THF (5 mL) at −78° C. After stirring at −78° C. for 1 h, the reaction mixture was quenched by adding H₂O and extracted with CH₂Cl₂ (2×100 mL). The extracts were dried over Na₂SO₄, concentrated at reduced pressure, and the residue was purified by flash chromatography to tert-butyl 7-(2-chloroacetyl)-2,3-dihydrobenzo[f][1,4]oxazepine-4(5H)-carboxylate (3.28 g, 57%).

To a stirred solution of tert-butyl 7-(2-chloroacetyl)-2,3-dihydrobenzo[f][1,4]oxazepine-4(5H)-carboxylate (2.28 g, 7.0 mmol) in CH₃CN (20 mL) was added LiBr (912 mg, 10.5 mmol), NaN₃ (910 mg, 14.0 mmol, in 6 mL of H₂O) and the mixture was stirred at 70° C. for 2 h. The reaction mixture was cooled to rt, concentrated at reduced pressure, and diluted with H₂O/CH₂Cl₂. The separated aqueous layer was extracted with CH₂Cl₂. The combined extracts were dried over Na₂SO₄, concentrated under reduced pressure, and the residue was purified by flash chromatography to afford tert-butyl 7-(2-azidoacetyl)-2,3-dihydrobenzo[f][1,4]oxazepine-4(5H)-carboxylate (2.11 g, 91%).

To a stirred solution of tert-butyl 7-(2-azidoacetyl)-2,3-dihydrobenzo[f][1,4]oxazepine-4(5H)-carboxylate (332 mg, 1.0 mmol) and tritylisothiocyanate (331 mg, 1.1 mmol) in 1,4-dioxane (10 mL) was added PPh₃ (289 mg, 1.1 mmol) and the mixture was stirred at reflux for 2 h. The reaction mixture was cooled to rt, concentrated under reduced pressure, and purification of the residue by flash chromatography gave tert-butyl 7-(2-aminooxazol-5-yl)-2,3-dihydrobenzo[f][1,4]oxazepine-4(5H)-carboxylate (306 mg, 57%).

To a stirred solution of tert-butyl 7-(2-aminooxazol-5-yl)-2,3-dihydrobenzo[f][1,4]oxazepine-4(5H)-carboxylate (306 mg) in 1,4-dioxane (6 mL) was added HCl (10 mL, 4 M in 1,4-dioxane) and the mixture was stirred at rt for 24 h. Solvents were removed under reduced pressure, treated with 7 N ammonia in MeOH (10 mL), and concentrated under reduced pressure. Purification of the residue by flash chromatography afforded 5-(2,3,4,5-tetrahydrobenzo[f][1,4]oxazepin-7-yl)oxazol-2-amine (98 mg, 80%).

To a stirred solution of 5-(2,3,4,5-tetrahydrobenzo[f][1,4]oxazepin-7-yl)oxazol-2-amine (46 mg, 0.2 mmol), acid (49 mg, 0.2 mmol), and DIPEA (105 μL, 0.6 mmol) in DMF (3 mL) was added HATU (76 mg, 0.2 mmol) and the mixture was stirred at rt for 10 min. Purification of the crude mixture by preparative HPLC gave [7-(2-amino-1,3-oxazol-5-yl)-2,3-dihydro-1,4-benzoxazepin-4(5H)-yl][2-ethyl-3-fluoro-4-(methylsulfonyl)phenyl]methanone (59 mg, 64%). ¹H NMR (400 MHz, DMSO-d₆): δ 7.78-7.71 (m, 1H), 7.48-7.48 (d, 0.5H), 7.34-7.33 (dd, 0.5H), 7.29-7.26 (m, 1H), 7.20 (s, 0.5H), 7.14-7.1d (d, 0.5H), 7.03 (bs, 1H), 7.03-7.01 (d, 1H), 6.92 (s, 0.5H), 6.84 (bs, 1H), 6.56-6.56 (d, 0.5H), 4.93-4.31 (m, 2H), 4.27-3.55 (m, 4H), 3.40-3.35 (d, 3H), 2.67-2.02 (m, 2H), 1.10-0.96 (m, 3H). MS (EI) for C₂₂H₂₂FN₃O₅S, found 460 (MH+).

### Example 14

### Methyl[6-(4-{[4-(methylsulfonyl)phenyl]carbonyl}-2,3,4,5-tetrahydro-1,4-benzoxazepin-7-yl)-1H-benzimidazol-2-yl]carbamate

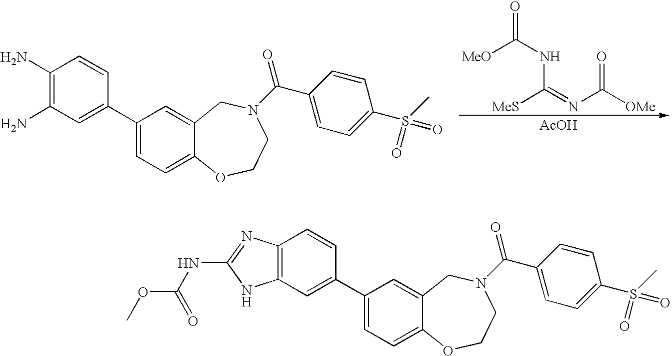

The mixture of (7-(3,4-diaminophenyl)-2,3-dihydrobenzo[f][1,4]oxazepin-4(5H)-yl)(4-(methylsulfonyl)phenyl)methanone (142 mg, 0.30 mmol), prepared as described in Example 3, and 1,3-bis(methoxycarbonyl)-2-methyl-2-thiopseudourea (62 mg, 0.3 mmol) in AcOH (4 mL) were stirred at 90° C. for 2 h. The reaction mixture was cooled to rt. Purification by preparative HPLC afforded methyl[6-(4-{[4-(methylsulfonyl)phenyl]carbonyl}-2,3,4,5-tetrahydro-1,4-benzoxazepin-7-yl)-1H-benzimidazol-2-yl]carbamate (129 mg, 76%). ¹H-NMR (400 MHz, DMSO-d₆): δ 11.63 (bs, 2H), 8.04-7.35 (m, 8H), 7.14-6.78 (m, 2H), 4.87-4.51 (d, 2H), 4.27-4.14 (m, 2H), 4.05-3.71 (m, 2H), 3.76 (s, 3H), 3.28-3.26 (d, 3H). MS (EI) for C₂₆H₂₄N₄O₆S, found 520.9 (MH+).

### Example 15

### 5-(4-{[4-(Methylsulfonyl)phenyl]carbonyl}-2,3,4,5-tetrahydro-1,4-benzoxazepin-7-yl)-1,3-dihydro-2H-benzimidazol-2-one

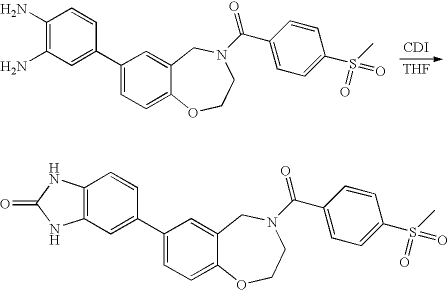

To a stirred solution of (7-(3,4-diaminophenyl)-2,3-dihydrobenzo[f][1,4]oxazepin-4(5H)-yl)(4-(methylsulfonyl)phenyl)methanone (17.5 mg, 0.04 mmol), prepared as described in Example 3, in THF (2 mL) was added CDI (13 mg, 0.08 mmol) and the mixture was stirred for overnight at room temperature. Solvents were removed under reduced pressure and the residue was purified by preparative HPLC to give 5-(4-{[4-(methylsulfonyl)phenyl]carbonyl}-2,3,4,5-tetrahydro-1,4-benzoxazepin-7-yl)-1,3-dihydro-2H-benzimidazol-2-one (16 mg, 86%). ¹H-NMR (400 MHz, DMSO-d₆): δ 10.73-10.64 (m, 2H), 8.02-7.97 (m, 2H), 7.67-7.41 (m, 4H), 7.23-6.59 (m, 4H), 4.85-4.49 (d, 2H), 4.26-4.13 (m, 2H), 4.04-3.70 (m, 2H), 3.39-3.38 (d, 3H). MS (EI) for C₂₄H₂ᵢN₃O₅S, found 463.9 (MH+).

### Example 16

### [7-(1H-Benzotriazol-6-yl)-2,3-dihydro-1,4-benzoxazepin-4(5H)-yl][4-(methylsulfonyl)phenyl]methanone

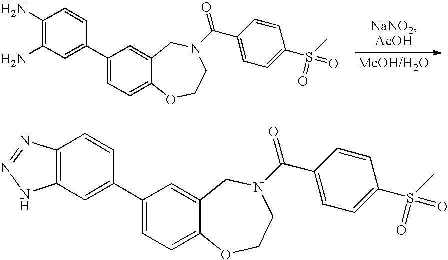

To a stirred solution of (7-(3,4-diaminophenyl)-2,3-dihydrobenzo[f][1,4]oxazepin-4(5H)-yl)(4-(methylsulfonyl)phenyl)methanone (66 mg, 0.15 mmol), prepared as described in Example 3, in MeOH/H₂O (3 mL/3 mL) was added AcOH (0.4 mL) and NaNO₂ (31 mg, 0.45 mmol). The mixture was stirred at rt for 30 min. The crude mixture was diluted with CH₂Cl₂, washed with H₂O, dried over Na₂SO₄, concentrated under reduced pressure. Purification of the residue by preparative HPLC gave [7-(1H-benzotriazol-6-yl)-2,3-dihydro-1,4-benzoxazepin-4(5H)-yl][4-(methylsulfonyl)phenyl]methanone (42 mg, 62%). ¹H-NMR (400 MHz, CDCl₃): δ 8.11-7.98 (M, 4), 7.92-7.81 (M, 1 h), 7.76-7.50 (m, 5H), 7.14-6.97 (m, 1H), 4.91-4.56 (d, 2H), 4.32-4.18 (m, 2H), 4.05-3.73 (m, 2H), 3.27 (s, 3H). MS (EI) for C₂₃H₂₀N₄O₄S, found 448.9 (MH+).

### Example 17

### 4-{[4-(Methylsulfonyl)phenyl]carbonyl}-7-[2-(methylthio)-1H-benzimidazol-6-yl]-2,3,4,5-tetrahydro-1,4-benzoxazepine

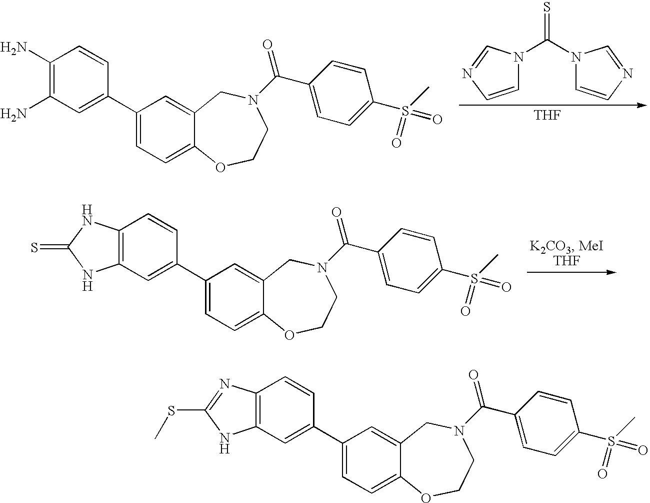

To a stirred solution of (7-(3,4-diaminophenyl)-2,3-dihydrobenzo[f][1,4]oxazepin-4(5H)-yl)(4-(methylsulfonyl)phenyl)methanone (317 mg, 0.72 mmol), prepared as described in Example 3, in THF (15 mL) was added 1,1]-thiocarbonyldiimidazole and the mixture was stirred at rt for 2 h. The crude mixture was purified by flash chromatography to afford (4-(methylsulfonyl)phenyl)(7-(2-thioxo-2,3-dihydro-1H-benzo[d]imidazol-5-yl)-2,3-dihydrobenzo[f][1,4]oxazepin-4(5H)-yl)methanone (317 mg, 91%).

To a stirred mixture of (4-(methylsulfonyl)phenyl)(7-(2-thioxo-2,3-dihydro-1H-benzo[d]imidazol-5-yl)-2,3-dihydrobenzo[f][1,4]oxazepin-4(5H)-yl)methanone (317 mg, 0.66 mmol) in THF (6 mL) was added K₂CO₃ (456 mg, 3.3 mmol) and iodomethane (82 μL, 1.32 mmol). The mixture was stirred at rt for 1 h. The crude mixture was directly purified by flash chromatography to give 4-{[4-(methylsulfonyl)phenyl]carbonyl}-7-[2-(methylthio)-1H-benzimidazol-6-yl]-2,3,4,5-tetrahydro-1,4-benzoxazepine (313 mg, 96%). ¹H NMR (400 MHz, DMSO-d₆): δ 12.64-12.51 (m, 1H), 8.01-7.51 (m, 7H), 7.42-6.81 (m, 3H), 4.87-4.53 (m, 2H), 4.28-4.14 (m, 2H), 4.05-3.71 (m, 2H), 3.29-3.27 (m, 3H), 2.71-2.70 (m, 3H). MS (EI) for C₂₅H₂₃N₃O₄S₂: 493.9 (MH+).

### Example 18

### 7-[2-(methylsulfonyl)-1H-benzimidazol-6-yl]-4-{[4-(methylsulfonyl)phenyl]carbonyl}-2,3,4,5-tetrahydro-1,4-benzoxazepine

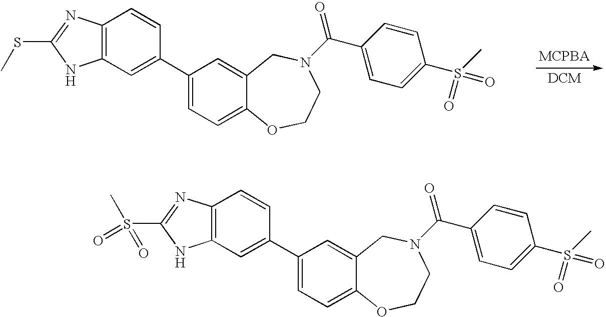

To a stirred solution of 4-{[4-(methylsulfonyl)phenyl]carbonyl}-7-[2-(methylthio)-1H-benzimidazol-6-yl]-2,3,4,5-tetrahydro-1,4-benzoxazepine (74 mg, 0.15 mmol), prepared as described in Example 17, in CH₂Cl₂ (8 mL) was added mCPBA (74%, 0.33 mmol) and the mixture was stirred at rt for 4 h. The reaction mixture was diluted with CH₂Cl₂, washed with aqueous saturated NaHCO₃, and the separated aqueous layer was extracted with CH₂Cl₂ (2×50 mL). The combined extracts were dried over Na₂SO₄, concentrated under reduced pressure, and the residue was purified by preparative HPLC to give 7-[2-(methylsulfonyl)-1H-benzimidazol-6-yl]-4-{[4-(methylsulfonyl)phenyl]carbonyl}-2,3,4,5-tetrahydro-1,4-benzoxazepine (28 mg, 36%). ¹H-NMR (400 MHz, DMSO-d₆): δ 8.02-7.98 (M, 3H), 7.74-7.51 (m, 7H), 7.137-6.90 (m, 1H), 4.90-4.55 (d, 2H), 4.31-4.17 (m, 4H), 4.06-3.72 (m, 2H), 3.52-3.51 (d, 3H), 3.27 (s, 3H). MS (EI) for C₂₅H₂₃N₃O₆S₂: 525.8 (MH+).

### Example 19

### {7-[2-(Dimethylamino)-1H-benzimidazol-6-yl]-2,3-dihydro-1,4-benzoxazepin-4(5H)-yl}[4-(methylsulfonyl)phenyl]methanone

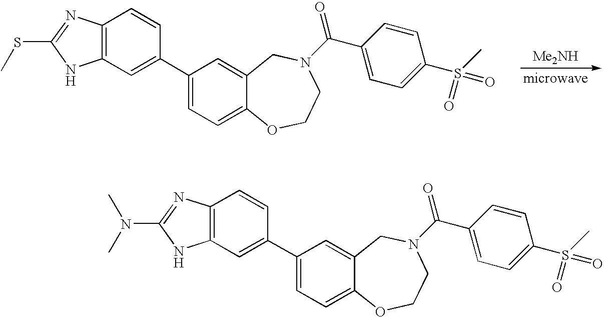

The mixture of 4-{[4-(methylsulfonyl)phenyl]carbonyl}-7-[2-(methylthio)-1H-benzimidazol-6-yl]-2,3,4,5-tetrahydro-1,4-benzoxazepine (74 mg, 0.15 mmol), prepared as described in Example 17, and Me₂NH (4 mL, 2M in MeOH) were stirred in microwave reactor (150° C.) for 18 h. The reaction mixture was concentrated under reduced pressure and the residue was purified by preparative HPLC to give {7-[2-(dimethylamino)-1H-benzimidazol-6-yl]-2,3-dihydro-1,4-benzoxazepin-4(5H)-yl}[4-(methylsulfonyl)phenyl]methanone (7 mg, 10%). ¹H-NMR (400 MHz, DMSO-d₆): δ 11.99 (bs, 1H), 8.02-7.97 (m, 2H), 7.67-7.41 (m, 4H), 7.24-7.16 (m, 2H), 7.07-6.57 (m, 2H), 4.86-4.51 (d, 2H), 4.26-4.13 (m, 2H), 4.05-3.71 (m, 2H), 3.30-3.27 (d, 3H), 3.09-3.07 (d, 6H). MS (EI) for C₂₆H₂₆N₄O₄S: 490.9 (MH+).

### Example 20

### [7-(2-Chloro-1H-benzimidazol-6-yl)-2,3-dihydro-1,4-benzoxazepin-4(5H)-yl][4-(methylsulfonyl)phenyl]methanone

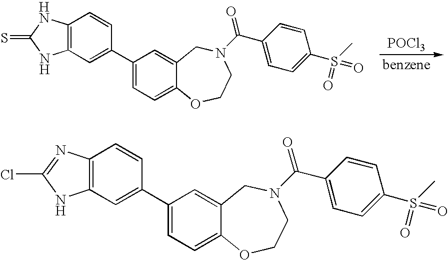

The mixture of (4-(methylsulfonyl)phenyl)(7-(2-thioxo-2,3-dihydro-1H-benzo[c/]imidazol-5-yl)-2,3-dihydrobenzo[f][1,4]oxazepin-4(5H)-yl)methanone (139 mg, 0.3 mmol), prepared as described in Example 17, and POCl₃ (8 mL) in benzene (15 mL) were refluxed for 18 h. The reaction mixture was cooled down to 0° C., quenched by adding H₂O carefully, diluted with CH₂Cl₂, washed with aqueous saturated NaHCO₃, dried over Na₂SO₄, concentrated under reduced pressure, and the residue was purified by preparative HPLC to afford [7-(2-chloro-1H-benzimidazol-6-yl)-2,3-dihydro-1,4-benzoxazepin-4(5H)-yl][4-(methylsulfonyl)phenyl]methanone (12 mg, 8%). ¹H-NMR (400 MHz, DMSO-d₆): δ 13.32 (bs, 1H), 8.01-7.95 (m, 2H), 7.73-7.50 (m, 6H), 7.29-6.86 (m, 2H), 4.88-4.53 (d, 2H), 4.29-4.16 (m, 2H), 4.05-3.72 (m, 2H), 3.26 (s, 3H). MS (EI) for C₂₄H₂₀ClN₃O₄S: 481.9 (MH+).

### Example 21

### 5-(4-(2-Ethyl-3-fluoro-4-(methylsulfonyl)benzoyl)-2,3,4,5-tetrahydrobenzo[f][1,4]oxazepin-7-yl)-1,3,4-oxadiazol-2(3H)-one

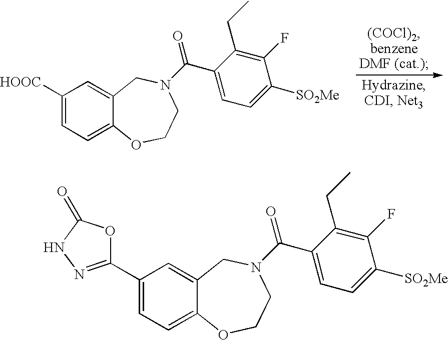

To 4-(2-ethyl-3-fluoro-4-(methylsulfonyl)benzoyl)-2,3,4,5 tetrahydrobenzo[f][1,4]oxazepine-7-carboxylic acid (50 mg, 0.12 mmol), prepared using procedures as described in Reference Example 9, in benzene (2 mL) was added (COCl)₂ (70 μL, 0.83 mmol) followed by DMF (cat., 1 drop) and the mixture was sonicated for 5 min. then heated to reflux for <1 min to dissolve the remaining starting material. An additional aliquot of (COCl)₂ (70 μL, 0.83 mmol) was added and after stirring for 5 min. at ambient temperature the reaction mixture was concentrated. To the 4-(2-ethyl-3-fluoro-4-(methylsulfonyl)benzoyl)-2,3,4,5-tetrahydrobenzo[f][1,4]oxazepine-7-carbonyl chloride was added THF (5 mL) followed by hydrazine (70 μL, 2.23 mmol) and the reaction mixture was stirred for 5 min then triethyl amine (1 mL, 7.72 mmol), and CDI (300 mg, 1.85 mmol) was added. After stirring for 2 h at rt the crude product was purified directly by reverse phase HPLC to provide 17.0 mg (32%) as an off-white solid.

5-(4-(2-Ethyl-3-fluoro-4-(methylsulfonyl)benzoyl)-2,3,4,5-tetrahydrobenzo[f][1,4]oxazepin-7-yl)-1,3,4-oxadiazol-2(3H)-one. ¹H NMR (400 MHz, DMSO-d₆): δ 7.80 (d, 1H), 7.76-7.69 (m, 1H), 7.66-7.61 (m, 1H), 7.28 (d, 1H), 7.12 (dd, 1H), 7.04-6.99 (m, 1H), 4.93 (dd, 1H), 4.50-4.46 (m, 1H), 4.37-4.34 (m, 1H), 4.25-4.22 (m, 1H), 4.11-4.03 (m, 1H), 3.61-3.57 (m, 1H), 3.37-3.35 (m, 3H), 2.68-2.32 (m, 2H), 1.09-0.99 (m, 3H). MS (EI) for C₂₁H₂₀FN₃O₆S, found 460 (MH−).

### Example 22

### 7-(2-methyl-1H-benzimidazol-6-yl)-4-({4-[(3-morpholin-4-ylpropyl)sulfonyl]phenyl}carbonyl)-2,3,4,5-tetrahydro-1,4-benzoxazepine

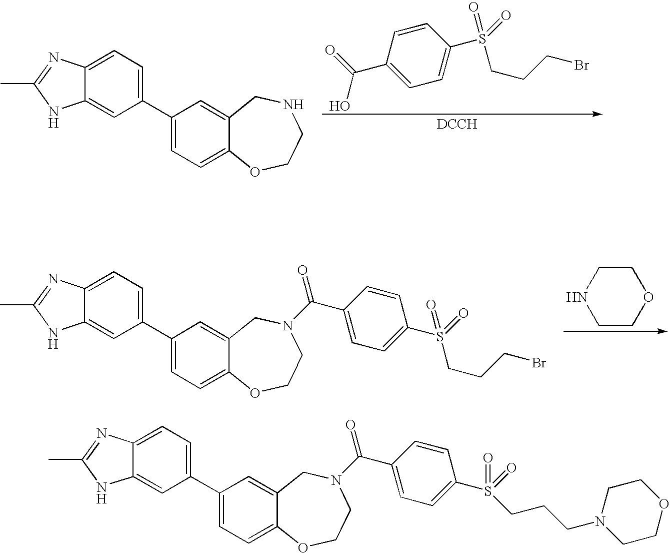

4-[(3-Bromopropyl)sulfonyl]benzoic acid (46 mg, 0.150 mmol), prepared using procedures as described in Reference Example 21, was dissolved in THF (1 mL) and was treated with DCC (31 mg, 0.150 mmol). 7-(2-methyl-1H-benzimidazol-6-yl)-2,3,4,5-tetrahydro-1,4-benzoxazepine dihydrochloride salt (53 mg, 0.151 mmol), prepared using procedures described in Reference Example 21, and diisopropylethylamine (0.052 mL, 0.3 mmol) were added and the mixture was stirred at ambient for 4 h. The mixture was filtered and the solid was washed with dichloromethane. The filtrate was washed with 0.2 N HCl. The aqueous portion was extracted with ethyl acetate. The combined organic portion was washed with saturated sodium bicarbonate solution, was dried over sodium sulfate, filtered and concentrated to afford (4-(3-bromopropylsulfonyl)phenyl)(7-(2-methyl-1H-benzo[d]imidazol-6-yl)-2,3-dihydrobenzo[f][1,4]oxazepin-4(5H)-yl)methanone as a colorless solid product which was used without further purification.

The (4-(3-bromopropylsulfonyl)phenyl)(7-(2-methyl-1H-benzo[d]imidazol-6-yl)-2,3-dihydrobenzo[f][1,4]oxazepin-4(5H)-yl)methanone from above was suspended in DMF (1.5 mL) and filtered. The filtrate solution was treated with morpholine (2 drops) at 60° C. for 3 h and was purified by preparative reverse phase HPLC (CH₃CN/H₂O). Acetonitrile was removed from the isolated pure fractions on a rotary evaporator and the aqueous remainder was lyophilized to give 7-(2-methyl-1H-benzimidazol-6-yl)-44 {4-[(3-morpholin-4-ylpropyl)sulfonyl]phenyl}carbonyl)-2,3,4,5-tetrahydro-1,4-benzoxazepine acetate salt as a colorless solid (19 mg, 0.030 mmol, 20% yield). ¹H NMR (400 MHz, d₆-DMSO): δ 7.96 (dd, 2H), 7.69-7.65 (m, 2H), 7.56-7.47 (m, 3H), 7.47-7.37 (m, 1H), 7.15 (d, 0.5H), 7.07 (t, 1H), 6.75 (br s, 0.5H), 4.88 (br s, 1H), 4.50 (br s, 1H), 4.30-4.23 (m, 1H), 4.18-4.12 (m, 1H), 4.08-4.01 (m, 1H), 3.74-3.68 (m, 1H), 3.54-3.42 (m, 6H), 3.40-3.33 (m, 3H), 2.32-2.11 (m, 6H), 1.76-1.61 (m, 2H). MS (EI) for C₃₁H₃₄N₄O₅S: 575.1 (MH⁺).

### Example 23

### 1-methyl-5-(7-(2-methyl-1H-benzo[d]imidazol-6-yl)-2,3,4,5-tetrahydrobenzo[f][1,4]oxazepine-4-carbonyl)-N-(3-morpholinopropyl)-1H-pyrrole-2-carboxamide

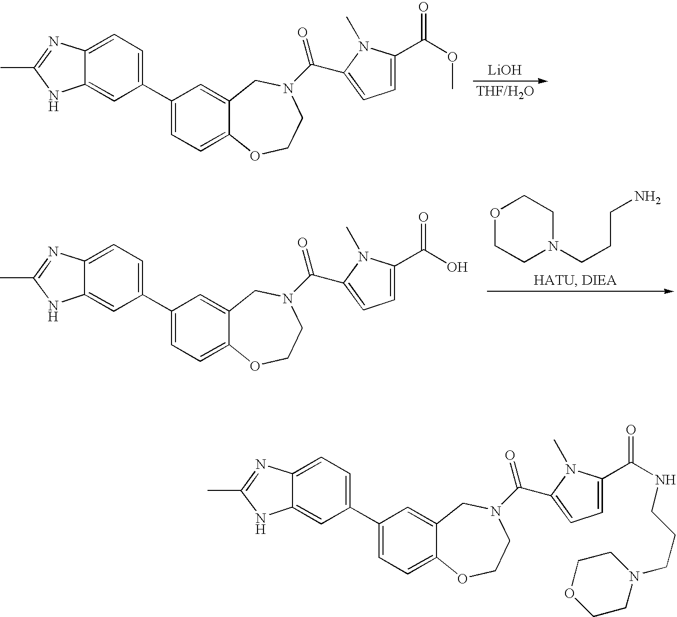

1-methyl-5-(7-(2-methyl-1H-benzo[d]imidazol-6-yl)-2,3,4,5-tetrahydrobenzo[f][1,4]oxazepine-4-carbonyl)-1H-pyrrole-2-carboxylic acid. To a solution of methyl 1-methyl-5-(7-(2-methyl-1H-benzo[d]imidazol-6-yl)-2,3,4,5-tetrahydrobenzo[f][1,4]oxazepine-4-carbonyl)-1H-pyrrole-2-carboxylate (0.388 g, 0.873 mmol), prepared as described in Example 2, in tetrahydrofuran (4.5 mL, THF), lithium hydroxide (0.042 g, 1.00 mmol, LiOH) in water (4.5 mL), and was added. The reaction mixture was heated at 80° C. for 1 h. The reaction was cooled to room temperature, the reaction was concentrated at reduced pressure, the resultant solid was azetroped with toluene (3×), affording 1-methyl-5-(7-(2-methyl-1H-benzo[d]imidazol-6-yl)-2,3,4,5-tetrahydrobenzo[f][1,4]oxazepine-4-carbonyl)-1H-pyrrole-2-carboxylic acid (0.301 g) which was used as such without purification. ¹H NMR (400 MHz, DMSO-d₆): δ 12.87-12.13 (m, 1H), 7.82-6.73 (m, 7H), 6.38-6.05 (m, 1H), 4.91-4.58 (m, 2H), 4.20 (s, 2H), 4.06-3.46 (m, 5H); MS (EI) for C₂₄H₂₂N₄O₄: 431.2 (MH+).

1-Methyl-5-(7-(2-methyl-1H-benzo[d]imidazol-6-yl)-2,3,4,5-tetrahydrobenzo[f][1,4]oxazepine-4-carbonyl)-N-(3-morpholinopropyl)-1H-pyrrole-2-carboxamide. To a mixture of 1-methyl-5-(7-(2-methyl-1H-benzo[d]imidazol-6-yl)-2,3,4,5-tetrahydrobenzo[f][1,4]oxazepine-4-carbonyl)-1H-pyrrole-2-carboxylic acid lithium salt (0.08 g, 0.183 mmol), N-(3-aminopropyl)morpholine (0.021 g, 0.147 mmol), DIEA (0.071 g, 0.550 mmol), in DMF (0.7 mL) was added HATU (0.056 g, 0.147 mmol) at room temperature. The resulting reaction mixture was stirred for 5 min at room temperature, then diluted with methanol (3 mL), and purified by preparative HPLC to give 1-methyl-5-(7-(2-methyl-1H-benzo[d]imidazol-6-yl)-2,3,4,5-tetrahydrobenzo[f][1,4]oxazepine-4-carbonyl)-N-(3-morpholinopropyl)-1H-pyrrole-2-carboxamide (0.045 g); ¹H NMR (400 MHz, DMSO); δ 8.22 (s, 1H), 7.76-7.43 (m, 4H), 7.15-7.02 (m, 1H), 6.73 (s, 1H), 6.36-6.05 (m, 1H), 4.76 (s, 2H), 4.22 (s, 2H), 3.96 (s, 2H), 3.84-3.56 (m, 7H), 3.2 (m, 2H), 2.40-2.24 (m, 6H), 1.63 (m, 2H); MS (EI) for C31H36N6O4: 557.3 (MH+).

Using the same or analogous synthetic techniques and substituting with appropriate reagents, the following examples were prepared.

N-(1,1-Dimethylethyl)-1-methyl-5-{[7-(2-methyl-1H-benzimidazol-6-yl)-2,3-dihydro-1,4-benzoxazepin-4(5H)-yl]carbonyl}-1H-pyrrole-2-carboxamide. ¹H NMR (400 MHz, DMSO-d₆): δ 12.24 (s, 1H), 7.80-7.22 (m, 6H), 7.07 (m, 1H), 6.71 (s, 1H), 4.76 (s, 2H), 4.21 (s, 2H), 3.96 (s, 2H), 3.63 (s, 3H), 1.33 (s, 9H); MS (EI) for C₂₈H₃ᵢN₅O₃: 486.3 (MH+).

N,1-Dimethyl-5-{[7-(2-methyl-1H-benzimidazol-6-yl)-2,3-dihydro-1,4-benzoxazepin-4(5H)-yl]carbonyl}-1H-pyrrole-2-carboxamide. ¹H NMR (400 MHz, DMSO-d₆): δ 8.15 (m, 1H), 7.86-7.48 (m, 4H), 7.26-6.64 (m, 2H), 6.35-6.03 (m, 1H), 4.79 (s, 2H), 4.23 (s, 2H), 3.96 (s, 2H), 3.84-3.51 (m, 3H), 2.76-2.63 (m, 3H), 2.57 (s, 3H); MS (EI) for C₂₅H₂₅N₅O₃: 444.2 (MH+).

N,N,1-Trimethyl-5-{[7-(2-methyl-1H-benzimidazol-6-yl)-2,3-dihydro-1,4-benzoxazepin-4(5H)-yl]carbonyl}-1H-pyrrole-2-carboxamide. ¹H NMR (400 MHz, DMSO-d₆): δ 11.99 (s, 1H), 7.78-7.37 (m, 4H), 7.27-6.95 (m, 2H), 6.37-6.17 (m, 2H), 4.80 (s, 2H), 4.25 (s, 2H), 4.00 (s, 2H), 3.48 (s, 2H), 2.98 (s, 6H), 2.59 (s, 3H); MS (EI) for C₂₆H₂₇N₅O₃: 458.2 (MH+).

1-Methyl-5-{[7-(2-methyl-1H-benzimidazol-6-yl)-2,3-dihydro-1,4-benzoxazepin-4(5H)-yl]carbonyl}-1H-pyrrole-2-carboxamide. ¹H NMR (400 MHz, DMSO-d₆): δ 7.81-6.74 (m, 9H), 6.33-6.01 (m, 1H), 4.76 (s, 2H), 4.22 (s, 2H), 3.82-3.54 (m, 3H); MS (EI) for C₂₄H₂₃N₅O₃: 430.2 (MH+).

7-(2-Methyl-1H-benzimidazol-6-yl)-4-{[4-(pyrrolidin-1-ylcarbonyl)phenyl]carbonyl}-2,3,4,5-tetrahydro-1,4-benzoxazepine. ¹H NMR (400 MHz, DMSO-d₆): δ 7.66 (s, 1H), 7.59-7.54 (m, 6H), 7.34-6.77 (m, 3H), 4.86 (s, 1H), 4.53 (s, 1H), 4.26 (s, 1H), 4.15 (s, 1H), 4.03 (s, 1H), 3.76 (s, 1H), 3.50-3.43 (m, 4H), 1.89-1.63 (m, 4H); MS (EI) for C₂₉H₂₈N₄O₃: 481.2 (MH+).

7-(2-Methyl-1H-benzimidazol-6-yl)-4-{[4-(piperidin-1-ylcarbonyl)phenyl]carbonyl}-2,3,4,5-tetrahydro-1,4-benzoxazepine. ¹H NMR (400 MHz, DMSO-d₆): δ 12.23 (s, 1H), 7.64 (m, 1H), 7.56-7.24 (m, 2H), 7.23-6.77 (s, 2H), 4.84 (s, 1H), 4.52 (s, 1H), 4.24 (s, 1H), 4.14 (s, 1H), 4.01 (s, 1H), 3.75 (s, 1H), 3.56 (s, 3H), 1.67-1.17 (m, 6H); MS (EI) for C₃₀H₃₀N₄O₃: 495.2 (MH+).

4-{[7-(2-Methyl-1H-benzimidazol-6-yl)-2,3-dihydro-1,4-benzoxazepin-4(5H)-yl]carbonyl}-N-(1-methylethyl)benzamide. ¹H NMR (400 MHz, DMSO-d₆): δ 12.23 (s, 1H), 8.41-8.27 (m, 1H), 8.00-7.82 (m, 2H), 7.64 (s, 1H), 7.57-7.12 (m, 5H), 7.09-6.78 (m, 1H), 4.83 (s, 1H), 4.53 (s, 1H), 4.25 (s, 1H), 4.17-3.95 (m, 3H), 3.71 (s, 1H), 1.20-1.09 (m, 6); MS (EI) for C₂₈H₂₈N₄O₃: 469.2 (MH+).

N-Cyclopropyl-4-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 12.45 (s, 1H), 8.64-8.50 (m, 1H), 7.95-7.82 (m, 2H), 7.77-6.81 (m, 7H), 4.85 (s, H), 4.54 (s, 1H), 4.27 (s, 1H), 4.13 (s, 1H), 4.03 (s, 1H), 3.73 (s, 1), 2.86 (s, 1H), 0.70 (m, 2H), 0.57 (m, 2H); MS (EI) for C₂₈H₂₆N4O₃: 467.2 (MH+).

N-Ethyl-4-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 8.65-8.54 (m, 1H), 7.96-7.85 (m, 2H), 7.75-7.65 (m, 1H), 7.61-7.33 (m, 4H), 7.28-6.83 (m, 1H), 4.86 (s, 1H), 4.56 (s, 1H), 4.28 (s, 1H), 4.14 (s, 1H), 4.03 (s, 1), 3.74 (s, 1H), 2.58-2.52 (m, 3H), 1.18-1.07 (m, 3H); MS (EI) for C₂₇H₂₆N₄O₃: 455.0 (MH+).

4-{[7-(2-Methyl-1H-benzimidazol-6-yl)-2,3-dihydro-1,4-benzoxazepin-4(5H)-yl]carbonyl}-N-propylbenzamide. ¹H NMR (400 MHz, DMSO-d₆): δ 12.35-12.01 (m, 1H), 8.70-8.57 (m, 1H), 8.00-7.89 (m, 2H), 7.81-7.21 (m, 6H), 7.13-6.96 (m, 1H), 4.90 (s, 1H), 4.60 (s, 1H), 4.32 (s, 1), 4.18 (s, 1), 4.08 (s, 1H), 3.79 (s, 1H), 3.32-3.22 (m, 2H), 1.65-1.52 (m, 2H), 0.91 (t, 3H); MS (EI) for C₂₈H₂₈N₄O₃: 469.0 (MH+).

N-Cyclopentyl-4-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 12.24 (s, 1H), 8.48-8.34 (m, 1H), 8.03-7.84 (m, 2H), 7.76-7.14 (m, 6H), 7.11-6.80 (m, 1H), 4.85 (s, 1H), 4.54 (s, 1H), 4.32-4.18 (m, 2H), 4.13 (s, 1), 3.73 (s, 1), 1.95-1.82 (m, 2H); MS (EI) for C₃₀H₃₀N₄O₃: 495.0 (MH+).

N-Cyclohexyl-4-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 12.25 (s, 1H), 8.32 (m, 1H), 8.00-7.85 (m, 2H), 7.73-7.15 (m, 6H), 7.10-6.80 (m, 1H), 4.86 (s, 1H), 4.54 (s, 1H), 4.27 (s, 1H), 4.12 (s, 1H), 4.02 (s, 1H), 3.84-3.69 (m, 2H), 1.87-1.55 (m, 5H), 1.39-1.04 (m, 5H); MS (EI) for C₃ᵢH₃₂N₄O₃: 509.3 (MH+).

N-(1-Ethylpropyl)-4-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 12.23 (s, 1H), 8.21-8.08 (m, 1H), 7.98-7.82 (m, 2H), 7.74-7.11 (m, 6H), 7.07-6.79 (m, 1H), 4.83 (s, 1), 4.52 (s, 1H), 4.25 (s, 1H), 4.11 (s, 1H), 4.01 (s, 1H), 3.95-3.66 (m, 2H), 1.60-1.36 (m, 4H), 0.83 (t, 6H); MS (EI) for C₃₀H₃₂N₄O₃: 497.3 (MH+).

7-(2-Methyl-1H-benzimidazol-6-yl)-4-({4-[(4-methylpiperazin-1-yl)carbonyl]phenyl}carbonyl)-2,3,4,5-tetrahydro-1,4-benzoxazepine. ¹H NMR (400 MHz, DMSO-d₆): δ 12.23 (m, 1), 7.77-7.41 (m, 7H), 7.33-6.55 (m, 3H), 4.86 (s, 1H), 4.55 (s, 1H), 4.26 (s, 1H), 4.15 (s, 1H), 4.03 (s, 1H), 3.78 (s, 1H), 3.60 (s, 2H), 2.42-1.93 (m, 1H); MS (EI) for C₃₀H₃₁N₅O₃: 510.3 (MH+).

4-{[7-(2-Methyl-1H-benzimidazol-6-yl)-2,3-dihydro-1,4-benzoxazepin-4(5H)-yl]carbonyl}-N-(2-morpholin-4-ylethyl)benzamide. ¹H NMR (400 MHz, DMSO-d₆): δ 12.24 (s, 1H), 8.61-8.48 (m, 1H), 7.94-7.84 (m, 2H), 7.79-7.16 (m, 6H), 7.11-6.54 (m, 1H), 4.86 (s, 1H), 4.55 (s, 1H), 4.28 (s, 1H), 4.14 (s, 1H), 4.03 (s, 1H), 3.74 (s, 1H), 3.56 (m, 4H), 3.40 (m, 2H), 2.51-2.31 (m, 6H); MS (EI) for C₃₁H₃₃N₅O₄: 540.3 (MH+).

N-(1,1-Dimethylethyl)-4-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 12.25 (s, 1H), 7.94-7.80 (m, 3H), 7.76-7.30 (m, 6H), 7.24-6.80 (m, 1H), 4.85 (s, 1H), 4.54 (s, 1H), 4.27 (s, 1H), 4.12 (s, 1H), 4.03 (s, 1H), 3.76 (s, 1H), 1.43-1.18 (m, 9H); MS (EI) for C₃₁H₃₃N₅O₄: 540.3 (MH+).

4-{[7-(2-Methyl-1H-benzimidazol-6-yl)-2,3-dihydro-1,4-benzoxazepin-4(5H)-yl]carbonyl}-N-(2-pyrrolidin-1-ylethyl)benzamide. ¹H NMR (400 MHz, DMSO-d₆): δ 8.66-8.52 (m, 1H), 7.96-7.84 (m, 2H), 7.70-7.33 (m, 6H), 7.24-6.83 (m, 1H), 4.85 (s, 1H), 4.55 (s, 1H), 4.27 (s, 1H), 4.03 (s, 1H), 3.74 (s, 1H), 3.38 (m, 2H), 2.56 (m, 2H), 2.46 (m, 4H), 1.66 (m, 4H); MS (EI) for C₃₁H₃₃N₅O₄: 524.3 (MH+).

4-{[7-(2-Methyl-1H-benzimidazol-6-yl)-2,3-dihydro-1,4-benzoxazepin-4(5H)-yl]carbonyl}-N-(3-morpholin-4-ylpropyl)benzamide. ¹H NMR (400 MHz, DMSO-d₆): δ 8.69-8.54 (m, 1H), 7.97 (m, 2H), 7.66 (s, 1H), 7.56-7.16 (m, 5H), 7.11-6.82 (m, 1H), 4.85 (s, 1H), 4.55 (s, 1H), 4.27 (s, 1H), 4.13 (s, 1H), 4.03 (s, 1H), 3.73 (s, 1H), 3.37 (m, 2H), 2.45-2.27 (m, 6H), 1.59-1.15 (m, 6H); MS (EI) for C₃₂H₃₅N₅O₄: 554.3 (MH+).

4-{[7-(2-Methyl-1H-benzimidazol-6-yl)-2,3-dihydro-1,4-benzoxazepin-4(5H)-yl]carbonyl}-N-(2-piperidin-1-ylethyl)benzamide. ¹H NMR (400 MHz, DMSO-d₆): δ 12.24 (s, 1H), 8.60-8.44 (m, 1H), 7.97-7.8 (m, 2H), 7.79-7.13 (m, 6H), 7.11-6.82 (m, 1H), 4.85 (s, 1H), 4.55 (s, 1H), 4.27 (s, 1H), 4.13 (s, 1H), 4.03 (s, 1H), 3.73 (s, 1H), 3.37 (m, 2H), 2.45-2.27 (m, 6H), 1.59-1.15 (m, 6H). MS (EI) for C₃₂H₃₅N₅O₃, found 538 (MH+).

N-Cyclopentyl-3-{[7-(2-methyl-1H-benzimidazol-6-yl)-2,3-dihydro-1,4-benzoxazepin-4(5H)-yl]carbonyl}benzamide. ¹H NMR (400 MHz, DMSO-d₆): δ 12.25 (s, 1H), 8.48-8.30 (m, 1H), 8.06-7.15 (m, 8H), 7.12-6.74 (m, 1H), 4.87 (s, 1H), 4.51 (s, 1H), 4.35-3.96 (m, 4H), 3.76 (s, 1H), 1.90-1.18 (m, 8H). MS (EI) for C₃₀H₃₀N₄O₃, found 495 (MH+).

### Example 24

### [7-{2-[(Cyclopropylamino)methyl]-1H-benzimidazol-6-yl}-2,3-dihydro-1,4-benzoxazepin-4(5H)-yl][2-ethyl-3-fluoro-4-(methylsulfonyl)phenyl]methanone

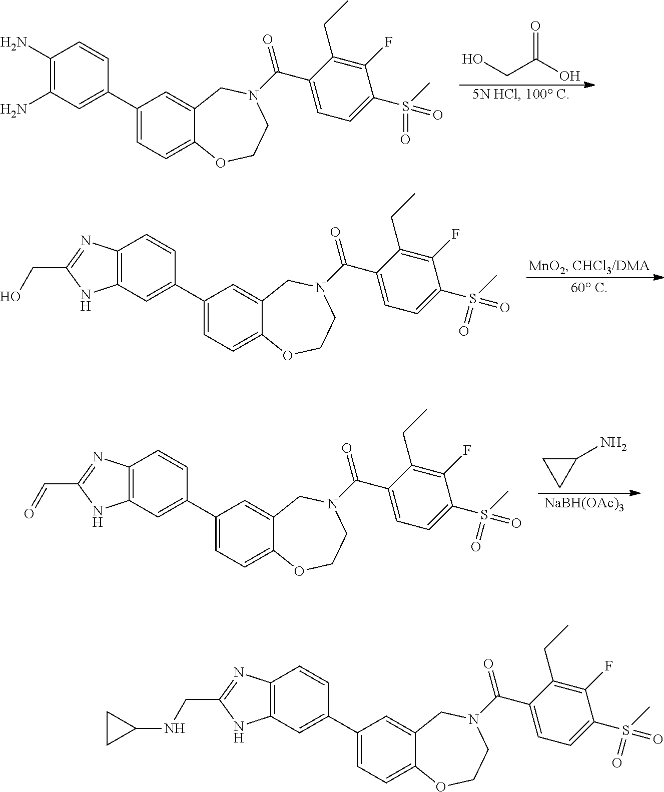

The mixture of (7-(3,4-diaminophenyl)-2,3-dihydrobenzo[f][1,4]oxazepin-4(5H)-yl)(2-ethyl-3-fluoro-4-(methylsulfonyl)phenyl)methanone (967 mg, 2.0 mmol), prepared using procedures as described in Example 3, and glycolic acid (183 mg, 2.4 mmol) in 5 N HCl (10 mL) were stirred at 100° C. for 16 h. The reaction mixture was cooled to rt, diluted with H₂O, and basified with 2 N NaOH, The precipitate was collected by suction filtration, and was dried under high vacuum to afford [2-ethyl-3-fluoro-4-(methylsulfonyl)phenyl]{7-[2-(hydroxymethyl)-1H-benzimidazol-6-yl]-2,3-dihydro-1,4-benzoxazepin-4(5H)-yl}methanone (617 mg, 59%). ¹H NMR (400 MHz, DMSO-d₆): δ 12.36 (bs, 1H), 7.80-7.67 (m, 2H), 7.55-7.44 (m, 3H), 7.30-6.75 (m, 3H), 5.05-4.38 (m, 2H), 4.70 (d, 2H), 4.34-3.55 (m, 4H), 3.35 (s, 3H), 2.70-2.04 (m, 2H), 1.12-0.97 (m, 3H). MS (EI) for C₂₇H₂₆FN₃O₅S: 524.2 (MH⁺).

To a stirred suspension of [2-ethyl-3-fluoro-4-(methylsulfonyl)phenyl]{7-[2-(hydroxymethyl)-1H-benzimidazol-6-yl]-2,3-dihydro-1,4-benzoxazepin-4(5H)-yl}methanone (439 mg, 0.84 mmol) in CHCl₃/DMA (15 mL/5 mL) was added MO₂ (1.46 g, 16.8 mmol) and the mixture was stirred at 60° C. for 2 h. The reaction mixture was cooled to rt and purified by flash chromatography to give 6-(4-(2-ethyl-3-fluoro-4-(methylsulfonyl)benzoyl)-2,3,4,5-tetrahydrobenzo[f][1,4]oxazepin-7-yl)-1H-benzo[d]imidazole-2-carbaldehyde (298 mg, 68%).

To a stirred solution of 6-(4-(2-ethyl-3-fluoro-4-(methylsulfonyl)benzoyl)-2,3,4,5-tetrahydrobenzo[f][1,4]oxazepin-7-yl)-1H-benzo[d]imidazole-2-carbaldehyde (52 mg, 0.1 mmol) and cyclopropylamine (21 μL, 0.3 mmol) in CH₂Cl₂ (6 mL) was added NaBH(OAc)₃ (53 mg, 0.25 mmol) and the resulting mixture was stirred at rt for 5 h. The reaction mixture was diluted with CH₂Cl₂, washed with aqueous NaHCO₃, and the separated aqueous layer was extracted with CH₂Cl₂ (2×100 mL). The combined extracts were dried over Na₂SO₄, concentrated under reduced pressure, and the residue was purified by preparative HPLC to give [7-{2-[(cyclopropylamino)methyl]-1H-benzimidazol-6-yl}-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 12.34-12.10 (m, 1H), 7.79-7.65 (m, 2H), 7.61-7.41 (m, 3H), 7.30-6.74 (m, 3H), 5.05-4.38 (m, 2H), 4.34-3.58 (m, 4H), 3.95 (d, 2H), 3.38-3.36 (d, 3H), 2.69-2.06 (m, 3H), 1.12-0.96 (m, 3H), 0.39-0.28 (m, 4H). MS (EI) for C₃₀H₃₁FN₄O₄S: 563.3 (MH⁺).

Using the same or analogous synthetic techniques and substituting with appropriate reagents, the following examples were prepared.

[6-(4-{[4-(Methylsulfonyl)phenyl]carbonyl}-2,3,4,5-tetrahydro-1,4-benzoxazepin-7-yl)-1H-benzimidazol-2-yl]methanol. ¹H NMR (400 MHz, DMSO-d₆): δ 12.43 (m, 1H), 7.98 (m, 2H), 7.67-7.52 (m, 6H), 7.08 (m, 1H), 5.77 (m, 1H), 4.88 (s, 1H), 4.70 (m, 2H), 4.52 (s, 1H), 4.28 (s, 1H), 4.15 (s, 1H), 3.72 (s, 1H), 3.27 (s, 3H). MS (EI) for C₂₅H₂₃N₃O₅S, found 478 (MH+).

4-{[2-Ethyl-3-fluoro-4-(methylsulfonyl)phenyl]carbonyl}-7-(2-{[(phenylmethyl)oxy]methyl}-1H-benzimidazol-6-yl)-2,3,4,5-tetrahydro-1,4-benzoxazepine. ¹H NMR (400 MHz, DMSO-d₆): δ 12.98 (br.m, 1H), 7.79-6.78 (m, 13H), 5.05-4.02 (m, 9H), 3.59 (m, 1H), 3.35 (m, 3H), 2.67 (m, 1H), 2.34-2.07 (m, 1H), 1.11-0.98 (m, 3H). MS (EI) for C₃₄H₃₂FN₃O₅S, found 614 (MH+).

[2-Ethyl-3-fluoro-4-(methylsulfonyl)phenyl]{7-[2-(pyrrolidin-1-ylmethyl)-1H-benzimidazol-6-yl]-2,3-dihydro-1,4-benzoxazepin-4(5H)-yl}methanone. ¹H NMR (400 MHz, DMSO-d₆): δ 12.40-12.28 (m, 1H), 7.80-7.67 (m, 2H), 7.61-7.41 (m, 3H), 7.30-6.75 (m, 3H), 5.05-4.38 (m, 2H), 4.35-3.57 (m, 4H), 3.83 (d, 2H), 3.38-3.36 (d, 3H), 2.70-2.04 (m, 6H), 1.76-1.72 (m, 4H), 1.12-0.97 (m, 3H). MS (EI) for C₃₁H₃₃FN₄O₄S: 577.3 (MH⁺).

[2-Ethyl-3-fluoro-4-(methylsulfonyl)phenyl][7-{2-[(propan-2-ylamino)methyl]-1H-benzimidazol-6-yl}-2,3-dihydro-1,4-benzoxazepin-4(5H)-yl]methanone. ¹H NMR (400 MHz, DMSO-d₆): δ 12.20 (bs, 1H), 7.80-7.67 (m, 2H), 7.55-7.42 (m, 3H), 7.30-6.75 (m, 3H), 5.05-4.38 (m, 2H), 4.34-3.55 (m, 4H), 3.92 (d, 2H), 3.38-3.36 (d, 3H), 2.79-2.72 (m, 1H), 2.69-2.03 (m, 2H), 1.76-1.72 (m, 4H), 1.12-0.96 (m, 9H). MS (EI) for C₃₀H₃₃FN₄O₄S: 565.3 (MH⁺).

[2-Ethyl-3-fluoro-4-(methylsulfonyl)phenyl][7-(2-{[(2-methoxyethyl)amino]methyl}-1H-benzimidazol-6-yl)-2,3-dihydro-1,4-benzoxazepin-4(5H)-yl]methanone. ¹H NMR (400 MHz, DMSO-d₆): δ 12.28-12.15 (bs, 1H), 7.80-7.67 (m, 2H), 7.59-7.42 (m, 3H), 7.30-6.75 (m, 3H), 5.05-4.38 (m, 2H), 4.35-3.57 (m, 4H), 3.94-3.92 (d, 2H), 3.44-3.41 (m, 2H), 3.38-3.36 (d, 3H), 3.25 (s, 3H), 2.73-2.70 (m, 2H), 2.68-2.06 (m, 2H), 1.12-0.96 (m, 3H). MS (EI) for C₃₀H₃₃FN₄O₅S: 581.3 (MH⁺).

[7-{2-[(Cyclopentylamino)methyl]-1H-benzimidazol-6-yl}-2,3-dihydro-1,4-benzoxazepin-4(5H)-yl][2-ethyl-3-fluoro-4-(methylsulfonyl)phenyl]methanone. ¹H NMR (400 MHz, DMSO-d₆): δ 12.23 (bs, 1H), 7.79-7.67 (m, 2H), 7.58-7.42 (m, 3H), 7.30-6.75 (m, 3H), 5.04-4.38 (m, 2H), 4.35-3.55 (m, 4H), 3.92-3.91 (d, 2H), 3.38-3.36 (d, 3H), 3.08-3.01 (m, 1H), 2.69-1.99 (m, 2H), 1.87-1.33 (m, 8H), 1.12-0.96 (m, 3H). MS (EI) for C₃₂H₃₅FN₄O₄S: 591.3 (MH⁺).

[7-(2-{[(Cyclopropylmethyl)amino]methyl}-1H-benzimidazol-6-yl)-2,3-dihydro-1,4-bezoxazepin-4(5H)-yl][2-ethyl-3-fluoro-4-(methylsulfonyl)-phenyl]methanone. ¹H NMR (400 MHz, DMSO-d₆): δ 12.27 (bs, 1H), 7.79-7.67 (m, 2H), 7.55-7.42 (m, 3H), 7.30-6.75 (m, 3H), 5.04-4.38 (m, 2H), 4.34-3.57 (m, 4H), 3.96-3.94 (d, 2H), 3.38-3.36 (d, 3H), 2.69-2.03 (m, 2H), 2.45-2.42 (dd, 2H), 1.12-0.96 (m, 3H), 0.95-0.90 (m, 1H), 0.43-0.38 (m, 2H), 0.14-0.10 (m, 2H). MS (EI) for C₃₂H₃₅FN₄O₄S: 591.3 (MH⁺).

### Biological Examples

Compounds of this invention have been tested using the assay described in Biological Example 1 and have been determined to be mTORc1 inhibitors. As such compounds of Formula I are useful for treating diseases, particularly cancer in which mTOR activity contributes to the pathology and/or symptomatology of the disease. Suitable in vitro assays for measuring mTORc1 and mTORc2 activity and the inhibition thereof by compounds, as well as cell-based assays for measurement of in vitro efficacy in treatment of cancer, are known in the art and examples are described below. Suitable in vivo models for cancer are known to those of ordinary skill in the art and examples are disclosed in below. Following the examples disclosed herein, as well as that disclosed in the art, a person of ordinary skill in the art can determine the mTOR-inhibitory activity of a compound of this invention.

### Biological Example 1

### mTOR/GbL/Raptor (mTORC1) ELISA Assay

The measurement of mTORC1 enzyme activity was performed in an ELISA assay format following the phosphorylation of 4E-BP1 protein. All experiments were performed in the 384-well format. Generally, 0.5 μL DMSO containing varying concentrations of the test compound was mixed with 15 μL enzyme solution. Kinase reactions were initiated with the addition of 15 μL of substrates-containing solution. The assay conditions were as follows; 0.2 nM mTORC1, 10 μM ATP and 50 nM NHis-tagged 4E-BP1 in 20 mM Hepes, pH 7.2, 1 mM DTT, 50 mM NaCl, 10 mM MnCl₂, 0.02 mg/mL BSA, 0.01% CHAPS, 50 mM β-glycerophosphate. Following an incubation of 120 minutes at ambient temperature, 20 μL of the reaction volume was transferred to a Ni-Chelate-coated 384-well plate. The binding step of the 4E-BP1 protein proceeded for 60 minutes, followed by washing 4 times each with 50 μL of Tris-buffered saline solution (TBS). Anti-phospho-4E-BP1 rabbit-IgG (20 μL, 1:5000) in 5% BSA-TBST (0.2% Tween-20 in TBS) was added and further incubated for 60 minutes. Incubation with a secondary HRP-tagged anti-IgG was similarly performed after washing off the primary antibody (4 washes of 50 μL). Following the final wash step with TBST, 20 μL of SuperSignal ELISA Femto (Pierce Biotechnology) was added and the luminescence measured using an EnVision plate reader.

All Compounds in Table 1 were tested in this assay and were found to inhibit mTOR at about 1.5 μM or less. In one embodiment, the Compound of the Invention demonstrated an inhibitory activity of 1.5 μM or less. In another embodiment, the Compound of the Invention demonstrated an inhibitory activity of 0.5 μM or less. In another embodiment, the Compound of the Invention demonstrated an inhibitory activity of 0.2 μM or less. In another embodiment, the Compound of the Invention demonstrated an inhibitory activity of 0.1 μM or less. In another embodiment, the Compound of the Invention demonstrated an inhibitory activity of 0.075 μM or less. In another embodiment, the Compound of the Invention demonstrated an inhibitory activity of 0.05 μM or less. In another embodiment, the Compound of the Invention demonstrated an inhibitory activity of 0.025 μM or less. In another embodiment, the Compound of the Invention demonstrated an inhibitory activity of 0.015 μM or less. In another embodiment, the Compound of the Invention demonstrated an inhibitory activity of 0.01 μM or less. In another embodiment, the Compound of the Invention demonstrated an inhibitory activity of 0.005 μM or less. In another embodiment, the Compound of the Invention demonstrated an inhibitory activity of 0.001 μM or less. In another embodiment, the Compound of the Invention demonstrated an inhibitory activity of 0.0005 μM or less. As numbered in Table 1, Compounds 844-880, 983-999 have an activity of greater than 500 nM and less than or equal to about 1500 nM. As numbered in Table 1, Compounds 717-843, 886, 961-982 have an activity of greater than 100 nM and less than or equal to about 500 nM. As numbered in Table 1, Compounds 428-560, 561-716, 884, 885, 887, 888, and 912-960 have an activity of greater than 10 nM and less than or equal to about 100 nM. As numbered in Table 1, Compounds 1-427, 566, 881-883, and 889-911 have an activity of less than or equal to about 10 nM. Activity for representative examples are listed below.

TABLE 2
Representative Biological Data

| | Activity in |
|---|---|
| | mTOR/GbL/Raptor |
| Compound Name | ELISA Assay (nM) |
| N-(azetidin-3-ylmethyl)-4-(4-{[2-ethyl-3-fluoro-4- | 9.9 |
| (methylsulfonyl)phenyl]carbonyl}-2,3,4,5-tetrahydro-1,4- | |
| benzoxazepin-7-yl)benzamide | |
| 4-(4-{[2-ethyl-3-fluoro-4-(methylsulfonyl)phenyl]carbonyl}-2,3,4,5- | 11.9 |
| tetrahydro-1,4-benzoxazepin-7-yl)-2-fluoro-N-methylbenzamide | |
| 3-[4-(4-{[2-ethyl-3-fluoro-4-(methylsulfonyl)phenyl]carbonyl}- | 14.4 |
| 2,3,4,5-tetrahydro-1,4-benzoxazepin-7-yl)phenyl]-1,1-dimethylurea | |
| 4-{[2-ethyl-3-fluoro-4-(methylsulfonyl)phenyl]carbonyl}-7-[4-(6- | 6 |
| methyl-1H-benzimidazol-2-yl)phenyl]-2,3,4,5-tetrahydro-1,4- | |
| benzoxazepine | |
| 2,2,2-trifluoro-1-[4-({7-[4-(1H-imidazol-2-yl)phenyl]-2,3-dihydro- | 3 |
| 1,4-benzoxazepin-4(5H)-yl}carbonyl)phenyl]ethane-1,1-diol | |
| N-[5-(4-{[2-ethyl-3-fluoro-4-(methylsulfonyl)phenyl]carbonyl}- | 5.3 |
| 2,3,4,5-tetrahydro-1,4-benzoxazepin-7-yl)-1,3-thiazol-2-yl]-N′- | |
| methylethane-1,2-diamine | |
| 4-{[7-(1H-benzimidazol-6-yl)-2,3-dihydro-1,4-benzoxazepin-4(5H)- | 5.6 |
| yl]carbonyl}-3-bromo-N-(1-methylethyl)benzenesulfonamide | |
| 4-[(3-chloro-1-ethyl-1H-indol-2-yl)carbonyl]-7-(2-methyl-1H- | 9.5 |
| benzimidazol-6-yl)-2,3,4,5-tetrahydro-1,4-benzoxazepine | |
| 7-(2-methyl-1H-benzimidazol-6-yl)-4-({4-[(4-methylpiperazin-1- | 8.6 |
| yl)sulfonyl]phenyl}carbonyl)-2,3,4,5-tetrahydro-1,4-benzoxazepine | |
| 7-(2-methyl-1H-benzimidazol-6-yl)-4-{[1-methyl-4-(pyrrolidin-1- | 7.1 |
| ylsulfonyl)-1H-pyrrol-2-yl]carbonyl}-2,3,4,5-tetrahydro-1,4- | |
| benzoxazepine | |
| 4-[(1,1-dioxido-2,3-dihydro-1-benzothien-5-yl)carbonyl]-7-(2-methyl- | 4.8 |
| 1H-benzimidazol-6-yl)-2,3,4,5-tetrahydro-1,4-benzoxazepine | |
| 4-{[7-(6-aminopyridin-3-yl)-2,3-dihydro-1,4-benzoxazepin-4(5H)- | 6.6 |
| yl]carbonyl}benzenesulfonamide | |
| 7-(1H-benzimidazol-6-yl)-4-[(2,4-dimethylphenyl)carbonyl]-2,3,4,5- | 6.4 |
| tetrahydro-1,4-benzoxazepine | |
| 7-(1H-indazol-6-yl)-4-{[4-(1,2,3-thiadiazol-4-yl)phenyl]carbonyl}- | 6.3 |
| 2,3,4,5-tetrahydro-1,4-benzoxazepine | |
| 4-[(4-{[2,5-bis(methyloxy)phenyl]sulfonyl}phenyl)carbonyl]-7-(2- | 3.2 |
| methyl-1H-benzimidazol-6-yl)-2,3,4,5-tetrahydro-1,4-benzoxazepine | |
| 4-[(2,4-dimethyl-4H-pyrrolo[3,2-d][1,3]thiazol-5-yl)carbonyl]-7-(2- | 17.2 |
| methyl-1H-benzimidazol-6-yl)-2,3,4,5-tetrahydro-1,4-benzoxazepine | |

### Biological Example 2

### Immune-Complex mTORC2 Kinase (mTORC2 IP-Kinase) Assay

HeLa (ATCC) cells were grown in suspension culture and lysed in ice-cold lysis buffer containing 40 mM HEPES pH 7.5, 120 mM NaCl, 1 mM EDTA, 10 mM sodium pyrophosphate, 10 mM β-glycerophosphate, 10 mM NaF, 10 mM NaN₃, one tablet of protease inhibitors (Complete-Mini, EDTA-free, Roche), 0.3% cholamidopropyldimethylammoniopropanesulfonate (CHAPS), 1 mM AEBSF, 0.5 mM benzamidine HCl, 20 μg/mL heparin, and 1.5 mM Na₃VO₄. The mTORC2 complex was immunoprecipitated with anti-RICTOR antibody for 2 h. The immune complexes were immobilized on Protein A sepharose (GE Healthcare, 17-5280-01), washed sequentially 3 times with wash buffer (40 mM HEPES pH 7.5, 120 mM NaCl, 10 mM β-glycerophosphate, 0.3% CHAPS, 1 mM AEBSF, 20 μg/mL heparin, 1.5 mM Na₃VO₄, and Complete-Mini, EDTA-free) and resuspended in kinase buffer (40 mM HEPES, pH 7.5, 120 mM NaCl, 0.3% CHAPS, 20 μg/mL heparin, 4 mM MgCl₂, 4 mM MnCl₂, 10% Glycerol, and 10 mM DTT). The immune complexes (equivalent to 1×10⁷ cells) were pre-incubated at 37° C. with a test compound or 0.6% DMSO for 5 min, and then subjected to a kinase reaction for 8 min in a final volume of 33 μL (including 5 μL bed volume) containing kinase buffer, 50 μM ATP, and 0.75 μg full length dephosphorylated AKT1. Kinase reactions were terminated by addition of 11 μL 4×SDS sample buffer containing 20% β-mercaptoethanol and resolved in a 10% Tris Glycine gels. The gels were transferred onto PVDF membrane at 50 V for 20 h at 4° C. The membranes were blocked in 5% non-fat milk in TBST for 1 h and incubated overnight at 4° C. with 1/1000 dilution of rabbit anti-pAKT (S473) (Cell Signaling Technology, 4060) in 3% BSA/TBST. The membranes were washed 3 times in TBST and incubated for 1 h with a 1/10000 dilution of secondary goat anti-rabbit HRP antibody (Cell Signaling Technology, 2125) in 5% non-fat milk/TBST. The signal was detected using Amersham ECL-plus. The scanned data were analyzed using ImageQuant software. IC₅₀ for the test compound was determined relative to DMSO treated sample using XLfit4 software.

Compounds of the Invention tested in this assay were found to have inhibitory activity for mTORc2. In one embodiment, the Compound of the Invention demonstrated an inhibitory activity of 200 nM or less.

### Biological Example 3

### pAKT (S473) ELISA Assay

PC-3 and MCF-7 cells (both from ATCC) were seeded onto 96-well plates (Corning, 3904) in DMEM (Cellgro, 10-013-CV) containing 10% FBS (Cellgro, 35-016-CV), 1% NEAA (Cellgro, 25-025-CI) and 1% penicillin-streptomycin (Cellgro, 30-002-CI) at 8×10³ cells per well (PC-3 cells) or 2.4×10⁴ cells per well (MCF-7 cells). Cells were incubated at 37° C., 5% CO₂ for 48 h, and the growth medium was replaced with serum-free DMEM. Serial dilutions of the test compound in 0.3% DMSO (vehicle) were added to the cells and incubated for 2 h and 50 min. Cells were then stimulated for 10 min with 20 ng/mL EGF (US Biologicals, E3374-07A)) for PC-3 or with 1.2 μg/mL Long R³ IGF-1 (Sigma, I1271) for MCF-7. To fix the cells, medium was removed and 100 μL/well of 4% formaldehyde (Sigma, F8775) in TBS (20 mM Tris, 500 mM NaCl) was added to each well at RT for 30 min. Cells were washed 3 times with 200 μL TBS containing 0.1% Tween 20 (Bio-Rad, 170-6351) (TBST), and quenched with 100 μL 0.6% H₂O₂ (VWR International, VW3742-1) in TBST for 30 min at RT. Plates were washed 3 times with 200 μL, TBST and blocked with 100 μL 5% BSA (Jackson ImmunoResearch, 001-000-173) in TBST for 1 h at RT. Anti-pAKT (S473) antibody (Cell Signaling Technology, 4058) or anti-total-AKT antibody (Cell Signaling Technology, 9272) were diluted 1/400 and 1/500, respectively, in 5% BSA in TBST. 50 μL of either primary antibody solution was added to the plate to detect pAKT (S473) or total AKT. After incubation overnight at 4° C., plates were washed 4 times with 200 μL TBST. Goat anti-rabbit secondary antibody (Jackson ImmunoResearch, 111-035-003) was diluted at 1/15000 in 5% BSA in TBST. 100 μL of antibody solution was added to each well and incubated for 1 h at RT. Plates were washed 3 times with 200 μL TBST and 2 times with 200 μL TBS. Chemiluminescent substrate (Super Signal Elisa Femto Chemiluminescent Substrate; Pierce, 37075) was prepared at RT. 100 μL of chemiluminescent substrate per well was added and then the plate was shaken for 1 min. Luminescence was read immediately on a Wallac plate reader at a wavelength of 560 nm. After normalization of pAKT signal to total AKT signal, IC₅₀ values were determined relative to the DMSO-treated control.

Compounds of the Invention tested in this assay in MCF7 cells were found to have an inhibitory activity of 500 nM or less.

Compounds of the Invention tested in this assay in PC-3 cells were found to have an inhibitory activity of 3 μM or less. In another embodiment, the Compound of the Invention demonstrated an inhibitory activity of 0.7 μM or less. In another embodiment, the Compound of the Invention demonstrated an inhibitory activity of 0.5 μM or less. In another embodiment, the Compound of the Invention demonstrated an inhibitory activity of 0.2 μM or less. In another embodiment, the Compound of the Invention demonstrated an inhibitory activity of 0.15 μM or less. In another embodiment, the Compound of the Invention demonstrated an inhibitory activity of 0.1 μM or less.

### Biological Example 4

### pS6 (S240/244) ELISA Assay

PC-3 and MCF-7 cells (both from ATCC) were seeded onto 96-well plates (Corning, 3904) in DMEM (Cellgro) containing 10% FBS (Cellgro), 1% NEAA (Cellgro) and 1% penicillin-streptomycin (Cellgro) at 8×10³ cells per well (PC-3 cells) or 2.4×10⁴ cells per well (MCF-7 cells). Cells were incubated at 37° C., 5% CO₂ for 48 h, and the growth medium was replaced with serum-free DMEM. Compounds were serially diluted in media containing a final concentration of 0.3% DMSO (vehicle). Compound dilutions were added to the cells and incubated for 3 h. To fix the cells, medium was removed and 100 μL/well of 4% formaldehyde (Sigma) in TBS was added to each well at RT for 30 min. Cells were washed 3 times with 200 μL TBST and quenched with 100 μL 0.6% H₂O₂ (VWR International) in TBST for 30 min at RT. Plates were washed 3 times with 200 μL TBST and blocked with 100 μL 5% BSA (Jackson ImmunoResearch) in TBST for 1 h at RT. Anti-pS6 (S240/244) antibody (Cell Signaling Technology, 2215) or anti-total-S6 antibody (Cell Signaling Technology, 2217) were diluted 1/500 in 5% BSA in TBST. 50 μL of either primary antibody solution was added to the plate to detect pS6 or total S6. After incubation overnight at 4° C., plates were washed 4 times with 200 μL TBST. Goat anti-rabbit secondary antibody (Jackson ImmunoResearch) was diluted at 1/15000 in 5% BSA in TBST. 100 μL of antibody solution was added to each well and incubated for 1 h at rt. Plates were washed 3 times with 200 μL TBST and 2 times with 200 μL TBS. Chemiluminescent substrate (Super Signal Elisa Femto Chemiluminescent Substrate) was prepared at rt. 100 μL of chemiluminescent substrate per well was added and then the plate was shaken for 1 min. Luminescence was read immediately on a Wallac plate reader at a wavelength of 560 nm. After normalization of pS6 signal to total S6 signal, IC₅₀ values were determined relative to the DMSO-treated control.

Compounds of the Invention tested in this assay in PC-3 cells were found to have an inhibitory activity of 3 μM or less. In another embodiment, the Compound of the Invention demonstrated an inhibitory activity of 1.5 μM or less. In another embodiment, the Compound of the Invention demonstrated an inhibitory activity of 1 μM or less. In another embodiment, the Compound of the Invention demonstrated an inhibitory activity of 0.7 μM or less. In another embodiment, the Compound of the Invention demonstrated an inhibitory activity of 0.5 μM or less. In another embodiment, the Compound of the Invention demonstrated an inhibitory activity of 0.3 μM or less. In another embodiment, the Compound of the Invention demonstrated an inhibitory activity of 0.2 μM or less. In another embodiment, the Compound of the Invention demonstrated an inhibitory activity of 0.1 μM or less.

Compounds of the Invention tested in this assay in MCF-7 cells has an inhibitory activity of 0.35 μM or less.

### Biological Example 5-7

### Pharmacodynamic Xenograft Tumor Models

Female and male athymic nude mice (NCr) 5-8 weeks of age and weighing approximately 20-25 g were used in the following models. Prior to initiation of a study, the animals were allowed to acclimate for a minimum of 48 h. During these studies, animals were provided food and water ad libitum and housed in a room conditioned at 70-75° F. and 60% relative humidity. A 12 h light and 12 h dark cycle was maintained with automatic timers. All animals were examined daily for compound-induced or tumor-related deaths.

### MCF-7 Breast Adenocarcinoma Model

MCF7 human mammary adenocarcinoma cells were cultured in vitro in DMEM (Cellgro) supplemented with 10% Fetal Bovine Serum (Cellgro), Penicillin-Streptomycin and non-essential amino acids at 37° C. in a humidified 5% CO₂ atmosphere. On day 0, cells were harvested by trypsinization, and 5×10⁶ cells in 100 μL of a solution made of 50% cold Hanks balanced salt solution with 50% growth factor reduced matrigel (Becton Dickinson) implanted subcutaneously into the hindflank of female nude mice. A transponder was implanted into each mouse for identification and data tracking, and animals were monitored daily for clinical symptoms and survival.

Tumors were established in female athymic nude mice and staged when the average tumor weight reached 100-200 mg. A Compound of the Invention was orally administered as a solution/fine suspension in water (with 1:1 molar ratio of 1 NHCL) once-daily (qd) or twice-daily (bid) at 10, 25, 50 and 100 mg/kg for 14 days. During the dosing period of 14-19 days, tumor weights were determined twice-weekly and body weights were recorded daily.

### Colo-205 Colon Model

Colo-205 human colorectal carcinoma cells were cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells were harvested by trypsinization, and 3×10⁶ cells (passage 10-15, >95% viability) in 0.1 mL ice-cold Hank's balanced salt solution were implanted intradermally in the hind-flank of 5-8 week old female athymic nude mice. A transponder was implanted in each mouse for identification, and animals were monitored daily for clinical symptoms and survival.

Tumors were established in female athymic nude mice and staged when the average tumor weight reached 100-200 mg. A Compound of the Invention was orally administered as a solution/fine suspension in water (with 1:1 molar ratio of 1 NHCL) once-daily (qd) or twice-daily (bid) at 10, 25, 50 and 100 mg/kg for 14 days. During the dosing period of 14 days, tumor weights were determined twice-weekly and body weights were recorded daily.

### PC-3 Prostate Adenocarcinoma Model

PC-3 human prostate adenocarcinoma cells were cultured in vitro in DMEM (Mediatech) supplemented with 20% Fetal Bovine Serum (Hyclone), Penicillin-Streptomycin and non-essential amino acids at 37° C. in a humidified 5% CO₂ atmosphere. On day 0, cells were harvested by trypsinization and 3×10⁶ cells (passage 10-14, >95% viability) in 0.1 mL of ice-cold Hank's balanced salt solution were implanted subcutaneously into the hindflank of 5-8 week old male nude mice. A transponder was implanted in each mouse for identification, and animals were monitored daily for clinical symptoms and survival.

Tumors were established in male athymic nude mice and staged when the average tumor weight reached 100-200 mg. A Compound of the Invention was orally administered as a solution/fine suspension in water (with 1:1 molar ratio of 1 N HCl) once-daily (qd) or twice-daily (bid) at 10, 25, 50, or 100-mg/kg for 19 days. During the dosing period of 14-19 days, tumor weights were determined twice-weekly and body weights were recorded daily.

Tumor weight (TW) in the above models is determined by measuring perpendicular diameters with a caliper, using the following formula:

tumor weight (mg)=[tumor volume=length (mm)×width² (mm²)]/2

These data were recorded and plotted on a tumor weight vs. days post-implantation line graph and presented graphically as an indication of tumor growth rates. Percent inhibition of tumor growth (TGI) is determined with the following formula:

$$[ 1 - ( \frac{( {X}_{f} - {X}_{0} )}{( {Y}_{f} - {X}_{0} )} ) ] * 100$$

where X₀=average TW of all tumors on group day

X_{f}=TW of treated group on Day f

Y_{f}=TW of vehicle control group on Day f

If tumors regress below their starting sizes, then the percent tumor regression is determined with the following formula:

$$( \frac{{X}_{0} - {X}_{f}}{{X}_{0}} ) * 100$$

Tumor size is calculated individually for each tumor to obtain a mean±SEM value for each experimental group. Statistical significance is determined using the 2-tailed Student's t-test (significance defined as P<0.05).

The foregoing invention has been described in some detail by way of illustration and example, for purposes of clarity and understanding. The invention has been described with reference to various specific embodiments and techniques. However, it should be understood that many variations and modifications may be made while remaining within the spirit and scope of the invention. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A compound of Formula I: 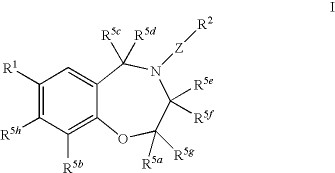
or a single stereoisomer or mixture of isomers thereof and additionally optionally as a pharmaceutically acceptable salt thereof, where
Z is —C(O)—;
R¹ is phenyl optionally substituted with one, two, or three R²⁰ groups independently selected from nitro; cyano; halo; alkyl; alkenyl; alkynyl; haloalkyl; —NR¹⁵R^{15a}; —NR¹⁵C(O)R¹⁸; —NR¹⁵S(O)₂R¹⁸; —NR¹⁵C(O)NR^{15a}R^{15b}; —OR⁹; —C(O)OR⁹; —C(O)R²⁶; —C(O)NR¹⁶R^{16a}; alkyl substituted with one or two —C(O)NR¹⁶R^{16a}; S(O)₂R¹⁷; heteroaryl optionally substituted with 1, 2, or 3 R²⁷; and optionally substituted heterocycloalkyl; or
R¹ is heteroaryl or an N-oxide thereof, optionally substituted with one, two, or three R²¹ groups independently selected from oxo; cyano; alkyl; alkenyl; alkynyl; halo; haloalkyl; hydroxyalkyl; alkoxy; alkoxyalkyl; optionally substituted cycloalkyl; optionally substituted cycloalkylalkyl; optionally substituted heterocycloalkyl; optionally substituted heterocycloalkylalkyl; optionally substituted heteroaryl; optionally substituted heteroarylalkyl; alkyl substituted with phenylalkyloxy; —OR²⁴; —SR²⁵; —S(O)R²⁵; —S(O)₂R²⁵; —S(O)₂NR¹⁵R^{15b}; —C(O)OR²²; —C(O)NR²³R^{23a}; —C(O)R^{24a}; —NR²³R^{23a}; alkyl substituted with one or two —NR²³R^{23a}; —NR²³C(O)OR^{24b}; —NR²³C(O)R^{23a}; alkyl substituted with one or two —NR²³C(O)R^{24a}; —NR²³C(O)NR^{23a}R²⁴; —NR²³C(═NH)NR^{23a}R²⁴; and —NR²³S(O)₂R^{23a};
R² is phenyl or naphthyl, each of which is substituted with R^{3a}, R^{3b}, R^{3c}, and R^{3d}; R² is HET¹ optionally substituted with R^{4a}, R^{4b}, and R^{4c}; or R² is HET² optionally substituted with R^{4a}, R^{4b}, R^{4c}, and R^{4d};
HET¹ is a 5- or 6-membered heteroaryl where the ring atom to which Z is attached is a carbon atom;
HET² is an 8- to 14-membered fused bicyclic ring containing one, two, three, or four ring heteroatoms independently selected from O, S, S(O), S(O)₂, and N, with the remaining ring atoms being carbon, where the ring atom attached to Z is carbon and where the ring attached to Z is aromatic and the other ring of HET² is partially or fully unsaturated;
R^{3a}, R^{3b}, R^{3c}, and R^{3d} are independently hydrogen; nitro; cyano; halo; alkyl; alkenyl; alkynyl; cyanoalkyl; haloalkyl; hydroxyalkyl; alkoxyalkyl; haloalkyl substituted with 1, 2, or 3 hydroxy; alkylsulfonylalkyl; —C(O)R²⁸; —C(O)NR¹³R^{13a}; —C(O)C(O)NR²⁹R^{29a}; —SR¹⁴; —S(O)R¹⁹; —S(O)₂R⁶; —S(O)₂NR⁷R^{7a}; —OR⁹; —NR¹¹R^{11a}; alkyl substituted with one or two —NR⁸R^{8a}; optionally substituted phenyl; optionally substituted phenylalkyl; optionally substituted heteroaryl; optionally substituted heteroarylalkyl; optionally substituted heterocycloalkyl; optionally substituted cycloalkyl; or optionally substituted cycloalkylalkyl;
R^{4a}, R^{4b}, R^{4c}, and R^{4d} are independently nitro; cyano; halo; oxo, alkyl; alkenyl; alkynyl; cyanoalkyl; haloalkyl; hydroxyalkyl; alkoxyalkyl; haloalkyl substituted with 1, 2, or 3 hydroxy; alkylsulfonylalkyl; —C(O)R¹²; —C(O)NR¹³R^{13a}; alkyl substituted with one or two groups independently selected from aminocarbonyl, alkylaminocarbonyl, and dialkylaminocarbonyl; —C(O)C(O)NR²⁹R^{29a}; —SR¹⁴; —S(O)R¹⁹; —S(O)₂R⁶; —S(O)₂NR⁷R^{7a}; —OR⁹; —NR¹¹R^{11a}; alkyl substituted with one or two —NR⁸R^{8a}; optionally substituted phenyl; optionally substituted phenylalkyl; optionally substituted heteroaryl; optionally substituted heteroarylalkyl; optionally substituted heterocycloalkyl; optionally substituted heterocycloalkylalkyl; optionally substituted cycloalkyl; or optionally substituted cycloalkylalkyl;
R^{5a} and R^{5c} are independently hydrogen, deuterium, or alkyl;
R^{5h} is hydrogen or halo;
R^{5b} is hydrogen, amino, or halo;
R^{5d}, R^{5e}, R^{5f}, and R^{5g} are independently hydrogen or deuterium;
R⁶ is halo; alkyl; alkenyl; alkynyl; haloalkyl; hydroxyalkyl; alkyl substituted with one or two —NR¹⁰R^{10a}; alkyl substituted with one heterocycloalkyloxy; optionally substituted phenyl; optionally substituted phenylalkyl; optionally substituted heterocycloalkyl; optionally substituted heterocycloalkylalkyl; optionally substituted cycloalkyl; or optionally substituted cycloalkylalkyl;
R⁷, R⁸, R¹⁰, R¹¹, R¹³, R¹⁵, R^{15b}, R¹⁶, R²⁹, and R^{29a} are independently hydrogen, alkyl, alkenyl, or alkynyl;
R^{7a} is hydrogen, alkoxy, alkyl, alkenyl, aminoalkyl, alkylaminoalkyl, dialkylaminoalkyl, hydroxyalkyl, alkylsulfonylalkyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, optionally substituted phenyl, or optionally substituted phenylalkyl;
R^{8a} and R^{10a} are independently hydrogen, alkyl, or alkoxycarbonyl;
R⁹ is hydrogen; alkyl; haloalkyl; hydroxyalkyl; optionally substituted phenyl; or alkyl substituted with one or two —NR¹⁰R^{10a};
R^{11a} is hydrogen, alkyl, alkenyl, alkynyl, alkoxycarbonyl, alkylsulfonyl, or optionally substituted phenylsulfonyl;
R¹² is alkyl, alkoxy, or hydroxy;
R^{13a} is hydrogen, alkyl, alkenyl, alkynyl, haloalkyl, optionally substituted cycloalkyl, optionally substituted cycloalkylalkyl, optionally substituted heterocycloalkyl, optionally substituted heterocycloalkylalkyl, optionally substituted phenyl, or optionally substituted phenylalkyl;
R¹⁴ and R¹⁹ are independently alkyl; haloalkyl; or optionally substituted phenyl;
R^{15a} is hydrogen, alkyl, alkenyl, alkynyl, haloalkyl, hydroxyalkyl, aminoalkyl, alkylaminoalkyl, or dialkylaminoalkyl;
R^{16a} is hydrogen, alkyl, alkenyl, alkynyl, haloalkyl, alkoxyalkyl, aminoalkyl, alkylaminoalkyl, dialkylaminoalkyl, optionally substituted heterocycloalkyl, or optionally substituted heterocycloalkylalkyl;
R¹⁷ is alkyl, alkenyl, alkynyl, amino, alkylamino, or dialkylamino;
R¹⁸ is alkyl, hydroxyalkyl, haloalkyl, aminoalkyl, alkylaminoalkyl, or dialkylaminoalkyl;
R²² and R²³ are independently hydrogen, alkyl, alkenyl, alkynyl, alkoxyalkyl, or haloalkyl;
R^{23a} is hydrogen, alkyl, alkenyl, alkynyl, alkoxyalkyl, aminoalkyl, alkylaminoalkyl, dialkylaminoalkyl, hydroxyalkyl, haloalkyl, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl;
R²⁴ is hydrogen, alkyl, aminoalkyl, alkylaminoalkyl, dialkylaminoalkyl, haloalkyl, hydroxyalkyl, or optionally substituted phenylalkyl;
R^{24a} is alkyl, haloalkyl, hydroxyalkyl, aminoalkyl, alkylaminoalkyl, dialkylaminoalkyl, or optionally substituted heterocycloalkyl;
R^{24b} is alkyl, haloalkyl, hydroxyalkyl, aminoalkyl, alkylaminoalkyl, or dialkylaminoalkyl;
R²⁵ is alkyl or haloalkyl;
R²⁶ is alkyl; or optionally substituted heterocycloalkyl;
each R²⁷, when R²⁷ is present, is independently selected from amino, alkylamino, dialkylamino, acylamino, halo, hydroxy, alkyl, haloalkyl, hydroxyalkyl, aminoalkyl, alkylaminoalkyl, dialkylaminoalkyl, aminocarbonyl, alkylaminocarbonyl, dialkylaminocarbonyl, or optionally substituted phenyl; and
R²⁸ is alkyl; haloalkyl; alkoxy; hydroxy; optionally substituted heterocycloalkyl; or optionally substituted phenyl.

2. The Compound according to claim 1 where R^{5a}, R^{5b}, R^{5c}, and R^{5h} are hydrogen; or a single stereoisomer or mixture of isomers thereof and additionally optionally as a pharmaceutically acceptable salt thereof.

3. The Compound according to claim 2 where R¹ is heteroaryl or an N-oxide thereof, optionally substituted with one, two, or three R²¹ groups; or a single stereoisomer or mixture of isomers thereof and additionally optionally as a pharmaceutically acceptable salt thereof.

4. The Compound according to claim 3 where the Compound of Formula I is according to Formula I(d) 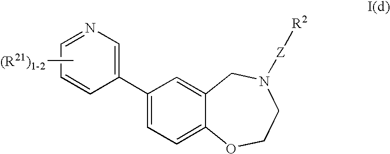
or a single stereoisomer or mixture of isomers thereof and additionally optionally as a pharmaceutically acceptable salt thereof.

5. The Compound according to claim 3 where R¹ is a 5-membered heteroaryl or an N-oxide thereof, optionally substituted with one, two, or three R²¹ groups independently selected from oxo, alkyl; halo; cyano; haloalkyl; hydroxyalkyl; alkoxy; alkoxyalkyl; optionally substituted cycloalkyl; optionally substituted cycloalkylalkyl; optionally substituted heterocycloalkyl; optionally substituted heterocycloalkylalkyl; optionally substituted heteroaryl; optionally substituted heteroarylalkyl; —C(O)OR²²; —NR²³R^{23a}; alkyl substituted with one —NR²³R^{23a}; —OR²⁴; —SR²⁵; —S(O)R²⁵; —S(O)₂R²⁵; —NR²³C(O)OR^{24a}; —NR²³C(O)R^{23a}; alkyl substituted with one —NR²³C(O)R^{24a}; alkyl substituted with arylalkyloxy; —C(O)NR²³R^{23a}; —C(O)R^{24a}; —NR²³C(O)NR^{23a}R²⁴; —NR²³C(═NH)NR^{23a}R²⁴; and —NR²³S(O)₂R^{23a}; or a single stereoisomer or mixture of isomers thereof and additionally optionally as a pharmaceutically acceptable salt thereof.

6. The Compound according to claim 3 where the Compound of Formula I is according to Formula I(e1) or I(e2) 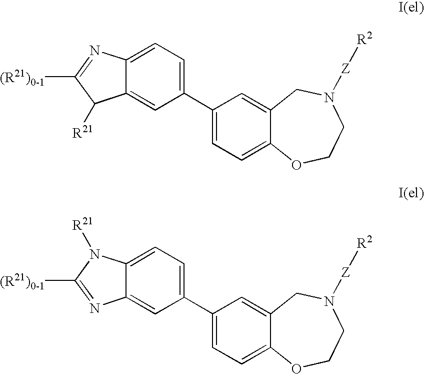
or a single stereoisomer or mixture of isomers thereof and additionally optionally as a pharmaceutically acceptable salt thereof.

7. The Compound according to claim 3 where the Compound of Formula I is according to Formula I(f) 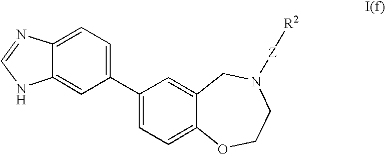
or a single stereoisomer or mixture of isomers thereof and additionally optionally as a pharmaceutically acceptable salt thereof.

8. The Compound according to claim 3 where the Compound of Formula I is according to Formula I(g) 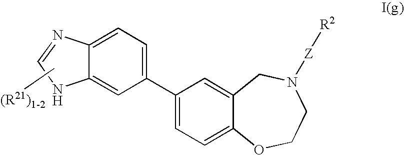
where each R²¹ is located at the 2-, 4-, or 5-positions of the benzimidazolyl ring; or a single stereoisomer or mixture of isomers thereof and additionally optionally as a pharmaceutically acceptable salt thereof.

9. The Compound according to claim 8 where the Compound of Formula I is according to Formula I(g) and one R²¹ is alkyl located at the 2-position of the R¹ benzimidazolyl and the second R²¹ is not present; or a single stereoisomer or mixture of isomers thereof and additionally optionally as a pharmaceutically acceptable salt thereof.

10. The Compound according to claim 3 where R¹ is pyrimidinyl, pyridazinyl, pyrazinyl, benzothiazolyl, benzoisoxazolyl, indolyl, 1H-pyrrolo[2,3-b]pyridinyl, indazolyl, 1H-pyrazolo[3,4-b]pyridinyl, 1H-imidazo[4,5-b]pyridinyl, or imidazo[1,2-a]pyridinyl; each of which is optionally substituted with one, two, or three R²¹ groups; or a single stereoisomer or mixture of isomers thereof and additionally optionally as a pharmaceutically acceptable salt thereof.

11. The Compound according to claim 2 where R¹ is phenyl optionally substituted with one, two, or three R²⁰ groups; or a single stereoisomer or mixture of isomers thereof and additionally optionally as a pharmaceutically acceptable salt thereof.

12. The Compound according to claim 11 where the Compound of Formula I is according to Formula I(k) 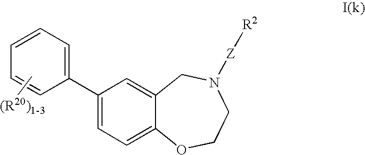
or a single stereoisomer or mixture of isomers thereof and additionally optionally as a pharmaceutically acceptable salt thereof.

13. The Compound according to claim 12 where the Compound of Formula I is according to Formula I(k) and one R²⁰ is selected from nitro; halo; alkyl; haloalkyl; —NR¹⁵R^{15a}; —NR¹⁵C(O)R¹⁸; —NR¹⁵S(O)₂R¹⁸; —OR⁹; heteroaryl optionally substituted with one or two R²⁷; —C(O)OR⁹; —C(O)R²⁶; —C(O)NR¹⁶R^{16a}; —NR¹⁵C(O)NR^{15b}R^{15a}; S(O)₂R¹⁷; alkyl substituted with —C(O)NR¹⁶R^{16a}; x and heterocycloalkyl optionally substituted with alkyl, alkoxycarbonyl, or phenylalkyl; the second R²⁰, when present, is selected from halo, alkyl, —NR¹⁵R^{15a}, and OR⁹; and the third R²⁰, when present, is halo; or a single stereoisomer or mixture of isomers thereof and additionally optionally as a pharmaceutically acceptable salt thereof.

14. The Compound according to claim 13 where R⁹ is hydrogen, alkyl, haloalkyl, or alkyl substituted with one or two —NR¹⁰R^{10a}; R¹⁰; R^{10a}; R¹⁵, and R¹⁶ is hydrogen or alkyl; R^{15a} is hydrogen, alkyl, haloalkyl, or dialkylaminoalkyl; R^{15b} is alkyl; R^{16a} is hydrogen, alkyl, haloalkyl, alkoxyalkyl, alkylaminoalkyl, dialkylaminoalkyl, optionally substituted heterocycloalkylalkyl, or heterocycloalkyl optionally substituted with alkyl; R¹⁷ is amino, alkylamino, or dialkylamino; R¹⁸ is alkyl, haloalkyl, or alkylaminoalkyl; each R²⁷, when present, is independently alkyl, haloalkyl, amino, acylamino, halo, hydroxy, hydroxyalkyl, aminoalkyl, alkylaminoalkyl, dialkylaminoalkyl, or optionally substituted phenyl; or a single stereoisomer or mixture of isomers thereof and additionally optionally as a pharmaceutically acceptable salt thereof.

15. The Compound according to any of claims 4, 6, 7, 8, 10, and 12 where R² is phenyl substituted with R^{3a}, R^{3b}, R^{3c}, and R^{3d}; or a single stereoisomer or mixture of isomers thereof and additionally optionally as a pharmaceutically acceptable salt thereof.

16. (canceled)

17. The Compound according to any of claim 15 where R² is according to formula (p) 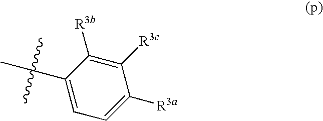
and R^{3a} is —S(O)R⁶; R^{3b} is alkyl or alkyl substituted with one —NR⁸R^{8a}; and R^{3c} is halo or —NR¹¹R^{11a}; or a single stereoisomer or mixture of isomers thereof and additionally optionally as a pharmaceutically acceptable salt thereof.

18. The Compound according to any of claim 15 where R² is according to formula (q) 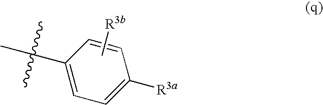
and R^{3a} is halo and R^{3b} is halo; R^{3a} is —S(O)₂R⁶ and R^{3b} is alkyl; R^{3a} is —S(O)₂R⁶ and R^{3b} is halo; R^{3a} is —S(O)₂R⁶ and R^{3b} is haloalkyl; R^{3a} is —S(O)₂NR⁷R^{7a} and R^{3b} is halo; R^{3a} is —S(O)₂NR⁷R^{7a} and R^{3b} is alkyl; R^{3a} is OR⁹ and R^{3b} is alkyl; R^{3a} is alkyl and R^{3b} is alkyl; R^{3a} is alkyl and R^{3b} is halo; R^{3a} is halo and R^{3b} is alkyl; R^{3a} is heteroaryl and R^{3b} is alkyl; R^{3a} is haloalkyl and R^{3b} is halo; R^{3a} is haloalkyl and R^{3b} is alkyl; or R^{3a} is —NR¹¹R^{11a} and R^{3b} is alkyl; or a single stereoisomer or mixture of isomers thereof and additionally optionally as a pharmaceutically acceptable salt thereof.

19. (canceled)

20. The Compound according to any of claim 15 where R² is according to formula (r) 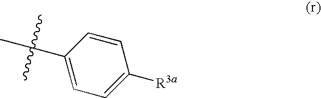
R^{3a} is nitro; cyano; halo; alkyl; alkynyl; cyanoalkyl; haloalkyl; haloalkyl substituted with 1, 2, or 3 hydroxy; alkylsulfonylalkyl; hydroxyalkyl; —C(O)R²⁸; —C(O)NR¹³R^{13a}; —C(O)C(O)NR²⁹R^{29a}; —SR¹⁴; —S(O)₂R⁶; S(O)₂NR⁷R^{7a}; —OR⁹; —NR¹¹R^{11a}; alkyl substituted with one —NR⁸R^{8a}; phenyl; heteroaryl optionally substituted with one alkyl or haloalkyl; heteroarylalkyl; heterocycloalkyl optionally substituted with one alkyl, or cycloalkyl; or a single stereoisomer or mixture of isomers thereof and additionally optionally as a pharmaceutically acceptable salt thereof.

21. (canceled)

22. The Compound according to any of claims 4, 6, 7, 8, 10, and 12 where R² is HET¹ optionally substituted with R^{4a}, R^{4b}, and R^{4c}; and R^{4a} is hydrogen; halo; alkyl; haloalkyl; —C(O)R¹²; —C(O)NR¹³R^{13a}; —S(O)₂R⁶; —S(O)₂NR⁷R^{7a}; —OR⁹; —NR¹¹R^{11a}; cycloalkyl; phenyl optionally substituted with 1 or 2 groups independently selected from halo, alkyl, alkylsulfonyl, and alkoxy; heteroaryl; heteroarylalkyl; or heterocycloalkyl optionally substituted with 1, 2, or 3 groups independently selected from alkyl and alkoxycarbonyl; R^{4b}, when R^{4b} is present, is hydrogen, alkyl, or haloalkyl; and R^{4c}, when R^{4c} is present, is hydrogen or alkyl; or mixture of isomers thereof and additionally optionally as a pharmaceutically acceptable salt thereof.

23. (canceled)

24. The Compound according to any of claims 4, 6, 7, 8, 10, and 12 where R² is HET² optionally substituted with R^{4a}, R^{4b}, and R^{4c}; and R^{4a}, when R^{4a} is present, is halo, alkyl, cyanoalkyl, alkoxyalkyl, —C(O)R¹², —OR⁹, —S(O)₂R⁶, cyanoalkyl, or phenyl; R^{4b}, when R^{4b} is present, is halo or alkyl; and R^{4c}, when R^{4c} is present, is halo; or mixture of isomers thereof and additionally optionally as a pharmaceutically acceptable salt thereof.

25. (canceled)

26. A Compound, as numbered in Table 1, according to claim 1 selected from
1
2
3
4
5
6
7
8
9
10
11
12
13
14
15
16
17
18
19
20
21
22
23
24
25
26
27
28
29
30
31
32
33
34
35
36
37
38
39
40
41
42
43
44
45
46
47
48
49
50
51
52
53
54
55
56
57
58
59
60
61
62
63
64
65
66
67
68
69
70
71
72
73
74
75
76
77
78
79
80
81
82
83
84
85
86
87
88
89
90
91
92
93
94
95
96
97
98
99
100
101
102
103
104
105
106
107
108
109
110
111
112
113
114
115
116
117
118
119
120
121
122
123
124
125
126
127
128
129
130
131
132
133
134
135
136
137
138
139
140
141
142
143
144
145
146
147
148
149
150
151
152
153
154
155
156
157
158
159
160
161
162
163
164
165
166
167
168
169
170
171
172
173
174
175
176
177
178
179
180
181
182
183
184
185
186
187
188
189
190
191
192
193
194
195
196
197
198
199
200
201
202
203
204
205
206
207
208
209
210
211
212
213
214
215
216
217
218
219
220
221
222
223
224
225
226
227
228
229
230
231
232
233
234
235
236
237
238
239
240
241
242
243
244
245
246
247
248
249
250
251
252
253
254
255
256
257
258
259
260
261
262
263
264
265
266
267
268
269
270
271
272
273
274
275
276
277
278
279
280
281
282
283
284
285
286
287
288
289
290
291
292
293
294
295
296
297
298
299
300
301
302
303
304
305
306
307
308
309
310
311
312
313
314
315
316
317
318
319
320
321
322
323
324
325
326
327
328
329
330
331
332
333
334
335
336
337
338
339
340
341
342
343
344
345
346
347
348
349
350
351
352
353
354
355
356
357
358
359
360
361
362
363
364
365
366
367
368
369
370
371
372
373
374
375
376
377
378
379
380
381
382
383
384
385
386
387
388
389
390
391
392
393
394
395
396
397
398
399
400
401
402
403
404
405
406
407
408
409
410
411
412
413
414
415
416
417
418
419
420
421
422
423
424
425
426
427
428
429
430
431
432
433
434
435
436
437
438
439
440
441
442
443
444
445
446
447
448
449
450
451
452
453
454
455
456
457
458
459
460
461
462
463
464
465
466
467
468
469
470
471
472
473
474
475
476
477
478
479
480
481
482
483
484
485
486
487
488
489
490
491
492
493
494
495
496
497
498
499
500
501
502
503
504
505
506
507
508
509
510
511
512
513
514
515
516
517
518
519
520
521
522
523
524
525
526
527
528
529
530
531
532
533
534
535
536
537
538
539
540
541
542
543
544
545
546
547
548
549
550
551
552
553
554
555
556
557
558
559
560
561
562
563
564
565
566
567
568
569
570
571
572
573
574
575
576
577
578
579
580
581
582
583
584
585
586
587
588
589
590
591
592
593
594
595
596
597
598
599
600
601
602
603
604
605
606
607
608
609
610
611
612
613
614
615
616
617
618
619
620
621
622
623
624
625
626
627
628
629
630
631
632
633
634
635
636
637
638
639
640
641
642
643
644
645
646
647
648
649
650
651
652
653
654
655
656
657
658
659
660
661
662
663
664
665
666
667
668
669
670
671
672
673
674
675
676
677
678
679
680
681
682
683
684
685
686
687
688
689
690
691
692
693
694
695
696
697
698
699
700
701
702
703
704
705
706
707
708
709
710
711
712
713
714
715
716
717
718
719
720
721
722
723
724
725
726
727
728
729
730
731
732
733
734
735
736
737
738
739
740
741
742
743
744
745
746
747
748
749
750
751
752
753
754
755
756
757
758
759
760
761
762
763
764
765
766
767
768
769
770
771
772
773
774
775
776
777
778
779
780
781
782
783
784
785
786
787
788
789
790
791
792
793
794
795
796
797
798
799
800
801
803
804
805
806
807
808
809
810
811
812
813
814
815
816
817
818
819
820
821
822
823
824
825
826
827
828
829
830
831
832
833
834
835
836
837
838
839
840
841
842
843
844
845
846
847
848
849
850
851
852
853
854
855
856
857
858
859
860
861
862
863
864
865
866
867
868
869
870
871
872
873
874
875
876
877
878
879
880
881
882
883
884
885
886
887
888
889
890
891
892
893
894
895
896
897
898
899
900
901
902
903
904
905
906
907
908
909
910
911
912
913
914
915
916
917
918
919
920
921
922
923
924
925
926
927
928
929
930
931
932
933
934
935
936
937
938
939
940
941
942
943
944
945
946
947
948
949
950
951
952
953
954
955
956
957
958
959
960
961
962
963
964
965
966
967
968
969
970
971
972
973
974
975
976
977
978
979
980
981
982
983
984
985
986
987
988
989
990
991
992
993
994
995
996
997
998
and
999
or a single stereoisomer or mixture of isomers thereof and additionally optionally as a pharmaceutically acceptable salt thereof.

27. A pharmaceutical composition which comprises a compound of claim 1 or 21 or a pharmaceutically acceptable salt thereof and a pharmaceutically acceptable carrier, excipient, or diluent.

28. A method of making a Compound of Formula I, according to claim 1 which method comprises
(a) reacting an intermediate of formula 6c, or a salt thereof: 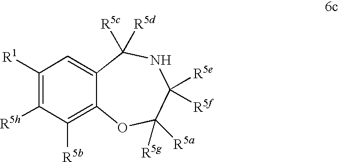
where R¹, R^{5a}, R^{5b}, R^{5c}, R^{5d}, R^{5e}, R^{5f}, R^{5g}, and R^{5h} are as defined in any of claim 1; with an intermediate of formula R²C(O)X where X is hydroxy or halo, and R² is as defined in claim 1 to yield a Compound of the Invention of Formula I; and optionally separating individual isomers; and optionally modifying any of the R¹ and R² groups; and optionally forming a pharmaceutically acceptable salt thereof; or
(b) reacting an intermediate of formula 40, or a salt thereof: 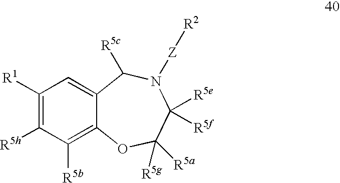
where R is halo or —B(OH)₂, and R^{5a}, R^{5b}, R^{5c}, R^{5d}, R^{5e}, R^{5f}, R^{5g}, and R^{5h} are as defined in claim 1; with an intermediate of formula R¹Y where Y is halo when R is —B(OH)₂ and Y is —B(OH)₂ when R is halo, and R² is as defined in claim 1 to yield a Compound of the Invention of Formula I; and optionally separating individual isomers; and optionally modifying any of the R¹ and R² groups; and optionally forming a pharmaceutically acceptable salt, hydrate, solvate or combination thereof.

29. A method for treating a disease, disorder, or syndrome which method comprises administering to a patient a therapeutically effective amount of a compound of claim 1 optionally as a pharmaceutically acceptable salt thereof, or a pharmaceutical composition comprising a compound of claim 1, optionally as a pharmaceutically acceptable salt thereof, and a pharmaceutically acceptable carrier, excipient, or diluent.

30. The method of claim 29 where the disease is cancer.

32. The method of claim 30 where the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, non small cell lung carcinoma, small cell carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, or head and neck cancer.

33. The method of claim 31 where the disease is hamaratoma, angiomyelolipomas, TSC-associated and sporadic lymphangioleiomyomatosis, multiple hamaratoma syndrome, neurofibromatosis, macular degeneration, macular edema, systemic lupus, or autoimmune lymphoproliferative syndrome.
